(19) United States
(12) Patent Application Publication
(10) Pub. No.: US 2023/0212674 A1
(43) Pub. Date: Jul. 6, 2023

(54) COMPOSITIONS AND METHODS FOR IDENTIFYING CELL TYPES

(71) Applicants: YISSUM RESEARCH DEVELOPMENT COMPANY OF THE HEBREW UNIVERSITY OF JERUSALEM LTD., JERUSALEM (IL); HADASIT MEDICAL RESEARCH SERVICES AND DEVELOPMENT LTD., JERUSALEM (IL); GRAIL, LLC, MENLO PARK, CA (US)

(72) Inventors: TOMER KAPLAN, JERUSALEM (IL); YUVAL DOR, JERUSALEM (IL); RUTH SHEMER, MEVASSERET ZION (IL); BENJAMIN GLASER, JERUSALEM (IL)

(21) Appl. No.: 18/147,647

(22) Filed: Dec. 28, 2022

Related U.S. Application Data
(60) Provisional application No. 63/295,319, filed on Dec. 30, 2021.

(51) Int. Cl. C12Q 1/6881; C12Q 1/6883

(52) U.S. Cl. CPC C12Q 1/6881; C12Q 1/6883; C12Q 2600/154

(57) ABSTRACT

The present disclosure relates generally to compositions and methods for determining cell type based on a methylation profile of associated DNA. For cell free DNA, such determination can be used to identify disease or conditions relating to the cell type. For tumor cells, such determination is useful for identifying their primary origin.

### CROSS REFERENCE TO RELATED APPLICATIONS

This application claims the benefit under 35 U.S.C. § 119(e) of the U.S. Provisional Application Ser. No. 63/295,319, filed Dec. 30, 2021, the content of which is hereby incorporated by reference in its entirety.

### REFERENCE TO AN ELECTRONIC SEQUENCE LISTING

The contents of the electronic sequence listing (334655US.xml; Size: 13,761,379 bytes; and Date of Creation: Dec. 27, 2022) is herein incorporated by reference in its entirety.

### BACKGROUND

Identification of the origin of a cell or cell free DNA has important implications. For instance, tumor cells may migrate to other tissues, making it challenging to identify their origin. Cancer of unknown primary origin (CUP) is a cancer that is determined to be at the metastatic stage at the time of diagnosis, but a primary tumor cannot be identified. CUP is found in about 3 to 5% of all people diagnosed with invasive cancer and carries a poor prognosis in most (80 to 85%) of those circumstances.

Small fragments of nucleic acids, e.g., DNA, circulate freely in the peripheral blood of healthy and diseased individuals. These cell-free nucleic acids, such as DNA (cfDNA) molecules may originate from dying or damaged cells and thus reflect ongoing cell death or injuries taking place in the body. In recent years, such understanding has led to the emergence of diagnostic tools, which are impacting multiple areas of medicine. For instance, next-generation sequencing of fetal DNA circulating in maternal blood has allowed non-invasive prenatal testing of fetal chromosomal abnormalities; detection of donor-derived DNA in the circulation of organ transplant recipients can be used for early identification of graft rejection; and the evaluation of mutated DNA in circulation can be used to detect genotype and monitor cancer.

Such technologies are powerful at identifying genetic anomalies in circulating DNA, or displaced cells, but are not informative when the DNA does not carry mutations. A key limitation with sequencing is that it does not reveal the tissue origins of the DNA, precluding the identification of tissue-specific cancer or cell death. The latter is critical in many settings such as neurodegenerative, inflammatory or ischemic diseases, not involving DNA mutations. Even in oncology, it is often important to determine the tissue origin of the tumor in addition to determining its mutational profile, for example in CUP and in the setting of early cancer diagnosis.

Identification of the tissue origins of DNA may also provide insights into collateral tissue damage (e.g., toxicity of drugs in genetically normal tissues), a key element in drug development and monitoring of treatment response.

### SUMMARY

The present disclosure provides compositions and methods for determining cell type based on methylation status of DNA fragments. Also provided are compositions and methods for identifying diseases and conditions in a subject, e.g., a human subject, through cell free DNA released by cells impacted by such diseases or conditions. In oncology or within another disease state, the present technology can be used to identify the primary origin of tumor cells.

In one embodiment, the present disclosure provides a method for identifying that a biological sample comprises DNA from a cell type. In some embodiment, the cell type is selected from the group of oral, larynx and esophageal epithelium, gastric epithelium, small intestine epithelium, colon epithelium, colon fibroblasts, gallbladder epithelium, liver hepatocytes, pancreatic acinar cells, pancreatic alpha cells, pancreatic beta cells, pancreatic delta cells, pancreatic ductal cells, endometrium epithelium, fallopian epithelium, kidney epithelium, bladder epithelium, prostate epithelium, breast basal epithelium, breast luminal epithelium, lung alveolar epithelium, lung bronchial epithelium, heart cardiomyocytes, heart fibroblasts, vascular endothelial cells, blood b cells, blood granulocytes, blood monocytes+macrophages, blood NK cells, blood t cells, erythrocyte progenitor cells, epidermal keratinocytes, dermal fibroblasts, osteoblasts, skeletal muscle cells, smooth muscle cells, thyroid epithelium, adipocytes, neuron CNS, and oligodendrocytes.

In some embodiments, the method entails detecting the methylation status of each of at least four, or at least five, six, seven, or eight CpG sites of a target DNA fragment in the biological sample and identifying the target DNA fragment as being from a human cell type when the methylation status of the target DNA fragment corresponds to the methylation status for the DNA fragment as defined in Table A for that cell type.

As used herein, in some embodiments, the methylation status refers to the percentage of CpG sites being methylated within the target DNA fragment (e.g., 25%). In some embodiments, the methylation status refers to whether the target DNA fragment is over-methylated (M, at least 60% CpG methylated) or under-methylated (U, no more than 40% CpG methylated) as compared to the same fragment in other cell types.

The target DNA fragment, in some embodiments, has the DNA sequence as shown in the accompanying Table B and Sequence Listing. As demonstrated in the experimental examples, however, the methylation pattern is uniform across a continuous region. Therefore, the sequences, or their genomic locations, are representative of the nearby genomic area.

In some embodiments, a target DNA fragment is one that includes at least a CpG site within a sequence included in the sequence listing. In some embodiments, a target DNA fragment is one that includes at least two CpG sites within a sequence included in the sequence listing. In some embodiments, a target DNA fragment is one that includes at least three or four CpG sites within a sequence included in the sequence listing.

In some embodiments, a target DNA fragment is within 1000 bp from either the 5′ end or 3′ end of a sequence included in the sequence listing. In some embodiments, a target DNA fragment is within 900, 800, 700, 600, 500, 400, 300, 250, 200 or 150 bp from either the 5′ end or 3′ end of a sequence included in the sequence listing.

In some embodiments, the target DNA fragment is obtained from a biological sample selected from the group consisting of blood, plasma, serum, semen, milk, urine, saliva and cerebral spinal fluid.

In some embodiments, the target DNA fragment is a cell-free DNA fragment. In some embodiments, identifying the cell-free DNA fragment as being from a cell type comprises detecting abnormal cell death of the cell type, or a disease relating to the cell type. In some embodiments, the method further entails identifying the human subject as having or likely having an injury, inflammation, or cancer at the corresponding cell type.

In some embodiments, the disease or condition is physical injury, inflammation, infection, cancer, diabetes, autoimmune disease, multiple sclerosis (MS), or a neurodegenerative disorder.

In some embodiments, the target DNA fragment has a length of 20-500 bp. In some embodiments, the target DNA fragment has a length of 30-400 bp, 40-300 bp, 50-250 bp, 50-200 bp, or 50-150 bp, without limitation.

In some embodiments, the methylation status is conversion of a cytosine to a 5-methylcytosine (5-mC) or to a 5-hydroxymethylcytosine (5-hmC). In some embodiments, detecting the methylation status comprises bisulfite or enzymatic treatment of the DNA fragment, or digestion of the DNA fragment with a restriction enzyme sensitive to DNA methylation. In some embodiments, the enzymatic treatment comprises treatment with APOBEC-Seq. In some embodiments, detecting the methylation status further comprises determining the sequence of the DNA fragment. In some embodiments, the sequence is determined by deep sequencing.

In some embodiments, the method further detecting a genetic variation in the target DNA fragment, thereby determining that the cell from which the target DNA fragment is released contains the genetic variation. In some embodiments, the method further comprises administering to the patient an agent useful for treating the identified disease or condition.

### BRIEF DESCRIPTION OF THE DRAWINGS

FIG. 1 presents a methylation atlas of the adult human body. 207 healthy samples were obtained from adult humans, isolated and deeply sequenced (WGBS, mean depth >30×), to form a comprehensive human cell type-specific methylation atlas.

FIG. 2 shows segmentation of the human genome into 7,264,350 continuous homogeneous blocks. The histograms show the number of segmented blocks as a function of their length in bases (left), or as a function of the number of CpGs they contain (right). In addition to the 2,746,623 blocks of length 3-30 CpGs (plotted above), there were additional 3,271,607 blocks of one CpG, and 1,185,719 blocks of two CpGs, as well 60,401 of >30 CpGs.

FIG. 3 shows biological replicates of the same cell type, from different individuals show a surprisingly low rate of differentially methylated blocks. This focused on 37 cellular subtypes with n≥3 replicates (e.g. endothelial cells from a specific tissue) and measured the average percent of methylation blocks (≥3 CpGs) that differ in their methylation by 50% (absolute delta beta), across replicates (shown as Y-axis). Nearly all cellular subtypes (36/37) differ by ≤0.5% of blocks suggesting a very high degree of conservation among replicates. Dotted red line marks the average number of differential blocks between two random samples of different cell types (4.9%).

FIG. 4 shows unsupervised agglomerative clustering reflects human developmental lineage of healthy cell types.

FIG. 5 shows average methylation in top differentially methylated blocks. Shown are the average methylation values at the 1% most variable blocks of 4 CpGs or more (21,077 blocks). For each block, we computed the average methylation in each sample, and classified them as unmethylated (<50%) or methylated (>50%). Boxplots show the 25th through 75th percentiles among the average methylation levels in unmethylated blocks/samples (blue), methylated ones (yellow) or the difference between methylated and unmethylated samples in the same block (green).

FIG. 6 show a Human Methylation Atlas of 207 samples across 39 cell types. (A) 953 genomic regions, unmethylated in a cell type-specific manner. Each cell in the plot marks the average methylation of one genomic region (column) at each of 39 cell types (rows). Up to 25 regions are shown per cell type, with a mean length of 251 bp (9 CpGs) per region. (B) Top 25 cardiomyocyte regions. For each region, the average methylation of each CpG site (columns) across all 207 samples is plotted in the atlas, and is grouped into 39 cell types as before. (C) A locus specifically unmethylated in cardiomyocytes. This marker (highlighted in light blue) is 120 bp long (6 CpGs), and is located in the first intron of MYL4, a heart-specific gene (TPM expression of 2518 in atrial appendage, GTEx inset). Genomic snapshot depicts average methylation (purple tracks) across six cardiomyocyte samples, four cardiac fibroblast samples, and three aorta samples (two endothelial, one smooth muscle cells). (D) Visualization of bisulfite converted fragments from three cardiomyocyte samples, one cardiac fibroblast sample, and two aorta samples (endothelium and smooth muscle). Shown are reads mapped to chr17:45289451-45289570 (hg19), with at least 3 covered CpGs. Yellow/blue dots depict methylated/unmethylated CpG sites.

FIG. 7 shows that cell type-specific markers are enriched for regulatory motifs. Shown are the top transcription factor binding site motifs, enriched among the top 250 differentially unmethylated regions per cell type, using HOMER motif analysis. Motifs similar to prior (more significant) hits are skipped.

FIG. 8 shows that cell type-specific hyper-methylated regions are enriched for CpG islands, polycomb targets, and CTCF and REST/NSRF. (A) 37.9% of top cell type-specific hyper-methylated markers (1,185 of 3,125, p<1E-100) overlap CpG islands. For comparison, 1.7% of cell type-specific hypo-methylated regions (198/11,371, p<2E-29) overlap CpG islands, which make up <0.9% of the genome (black line). (B) These regions are typically enriched for H3K27me3 in other cell types. Shown are the average H3K27me3 signals in monocytes and macrophages near all cell type-specific hyper-methylated regions (top, blue) or near monocytes/macrophages-specific hyper-methylated regions (green). (C) Similar plots for Polycomb annotations in monocytes and macrophages (chromHMM), for all or monocyte/macrophage-specific markers. (D) Motif analysis of cell type-specific hyper-methylated regions (top 100 per cell type) identifies known CTCF and REST/NSRF motifs. (E) Analysis of ChIP-seq data for one such site (chr1:209364093-209364250, highlighted in blue, hg19), specifically methylated in the small intestine and colon epithelium (box 1), and unmethylated elsewhere. As shown below, this site is bound in multiple cell types and tissues, but is mostly unbound in the stomach and colon epithelium, in vivo (box 2). (F) REST/NSRF motif is present within 15 of top 100 (15%) cell type-specific hyper-methylated regions in the endocrine pancreas (alpha, beta, and delta cells), 5 of top 100 pancreatic delta cells, and 2 of top 100 pancreatic beta cells, compared to ˜0.1% in background sequences, in accordance with REST target expression in the endocrine pancreas.

FIG. 9 shows the results of lung epithelium methylome analysis. A. Comparative tissue methylome analysis reveals multiple methylation blocks that are uniquely unmethylated in lung alveolar (1,663 blocks), bronchial epithelial cells (673 blocks), or both (139 blocks) and methylated in all other tissues. Additional 11 markers specifically methylated in the lung are not shown. Each marker covers ≥3 CpGs, and presents an average methylation delta of ≥0.4 between target cell type 25^{th} percentile and other tissues 97.5^{th} percentile. B. Characterization of one lung alveolar-specific methylation marker, located at chr16:667119-667272 (hg19), in the Rab40C gene. This region is unmethylated only in lung alveolar epithelium and is enriched for chromatin markers H3K27ac, H3K4me1 and H3K4me3. C. Lung-specific methylation markers are enriched for enhancer regions. For each of the three marker sets, shown is the number of markers with enhancer-related chromatin states in the lung, showing an enrichment of 2.5 to 10-fold change. D. GREAT annotations, identifying gene sets enriched among genes closest to lung-unique methylation markers. Shown are 5 of the most significant (BinomFDRQ) gene sets for the methylation markers of each lung cell type.

FIG. 10 shows the performance of the selected lung specific markers. A. Assay specificity. Methylation status of lung epithelial markers (alveolar in green, bronchial in orange and common lung in pink) in DNA from multiple tissues. Shown is the percentage of molecules in which most CpG sites were methylated or unmethylated. B. Assay specificity in Lung cancer. Methylation status of lung epithelial markers in DNA from multiple Lung Cancers. Shown is the percentage of molecules in which CpG sites were methylated or unmethylated according to the marker. The analysis is based on TCGA Illumina BeadCheap array data, where each locus is represented by one CpG site. Note that lung cancers retain methylation patterns of the normal lung. C. Assay sensitivity and accuracy in vitro. DNA from healthy human lung alveolar (left) or bronchial (right) epithelium was mixed with blood DNA as indicated, and the fraction of molecules methylated or unmethylated in the lung markers was determined. D. Assay robustness. cfDNA samples extracted from same donor in duplicates were analyzed for lung markers. Shown is the number of genome equivalents per ml plasma present in each duplicate.

FIG. 11 shows the testing results of lung-derived cfDNA in healthy individuals. A. Concentration of lung cfDNA in the plasma of 30 healthy donors. The concentration was measured by multiplying the fraction of lung cfDNA by the concentration of total cfDNA. B. Fraction of lung cfDNA in the plasma of 30 healthy donors and in lung lavages of 6 donors.

FIG. 12 shows identification of Lung-derived cfDNA in lung cancer patients. A. Lung cfDNA in the plasma of 26 patients with advanced lung cancer. The concentration was measured by multiplying the fraction of lung cfDNA by the concentration of total cfDNA. Dashed line in this panel and in C indicates average+2 standard errors of healthy controls. B. Lung cfDNA in the plasma of patients with lung cancer. Top, P value determined by 2-tailed Mann-Whitney test. Bottom, ROC curve of all advanced lung cancer patients vs. healthy samples. C. Lung cfDNA in the plasma of 51 donors undergoing bronchoscopy. The concentration was measured by multiplying the fraction of lung cfDNA by the concentration of total cfDNA. P value determined by 2-tailed Mann-Whitney test. Left, each color represents the cumulative value of markers for the indicated cell type. Right, each dot represents the cumulative value of all lung markers measured. D. Concentration of lung cfDNA in the plasma of donors undergoing bronchoscopy vs healthy patients (left), and a ROC curve for distinguishing patients with lung pathologies from healthy controls.

FIG. 13 shows the effect of number of lung markers on assay sensitivity. A. ROC curves using the indicated combination of lung methylation markers, for identifying patients with any lung pathology vs. healthy controls. B. Sensitivity of the indicated combination of lung markers at 70% specificity. Patients with lung pathologies vs healthy controls.

FIG. 14 shows the testing result of lung-specific cfDNA in patients with COPD. A. Concentration of lung cfDNA in the plasma of 77 patients with COPD. The concentration was measured by multiplying the fraction of lung cfDNA by the concentration of total cfDNA. Dashed line indicates average+2 standard errors of healthy controls. B. Lung cfDNA in the plasma of patients with lung cancer, exacerbated and stable COPD, and healthy controls. C. Lung cfDNA in the plasma of COPD patients that were still alive 14 months after sampling vs patients that died during this period.

FIG. 15 is a schematic illustrating the computing components that may be used to implement various features of the embodiments described in the present disclosure.

### DETAILED DESCRIPTION

The following description sets forth exemplary embodiments of the present technology. It should be recognized, however, that such description is not intended as a limitation on the scope of the present disclosure but is instead provided as a description of exemplary embodiments.

### Definitions

Unless defined otherwise, all technical and scientific terms used herein have the meaning commonly understood by a person skilled in the art to which this description belongs. As used herein, the following terms have the meanings ascribed to them below.

The term “methylation” as used herein refers to a process by which a methyl group is attached to a nucleic acid, e.g., DNA, molecule. For example, a hydrogen atom on the pyrimidine ring of a cytosine base can be converted to a methyl group, forming 5-methylcytosine. The term also includes a process by which a hydroxymethyl group is attached to a DNA molecule (specifically, “hydroxymethylation”), for example by oxidation of a methyl group on the pyrimidine ring of a cytosine base. Methylation, including hydroxymethylation, generally takes place at dinucleotides of cytosine and guanine referred to herein as “CpG dinucleotides” or “CpG sites.” The principles described herein are also applicable for the detection of methylation in a non-CpG context, including non-cytosine methylation. In such embodiments, a wet laboratory assay used to detect methylation may vary from any described herein. Further, the methylation state vectors may contain elements that are generally vectors of sites where methylation has or has not occurred (even if those sites are not CpG sites specifically).

The term “methylation site” as used herein refers to a region of a DNA molecule where a methyl group can be attached to the DNA molecule. “CpG” sites are the most common methylation site, but methylation sites are not limited to CpG sites. For example, DNA methylation may occur in cytosines in CHG and CHH, where H is adenine, cytosine or thymine.

The term “CpG site” as used herein refers to a region of a DNA molecule where a cytosine nucleotide is followed by a guanine nucleotide in the linear sequence of bases along its 5′ to 3′ direction. “CpG” is a shorthand for 5′-C-phosphate-G-3′ that is cytosine and guanine separated by only one phosphate group. Cytosines in CpG dinucleotides can be methylated to form 5-methylcytosine.

The term “under-methylated” or “over-methylated” as used herein refers to a methylation status of a DNA molecule containing multiple CpG sites (e.g., 2 or more, 3 or more, 4 or more, 5 or more, 6 or more, 7 or more, 8 or more, 9 or more, or 10 or more, etc.) where a higher percentage of the CpG sites (e.g., 5% or more, 10% or more, 15% or more, 20% or more, 25% or more, 30% or more, 40% or more, 50% or more, 70% or more, 75% or more, 80% or more, 85% or more, 90% or more, or 95% or more, or 97.5% or more, 98% or more, 99% or more, or 99.9% or more, or any other numerical percentage within the range 0% to 50% or within the range 50%-100%, wherein each provided range of the subject disclosure includes the range limit endpoints, e.g., 50% and 100%) are unmethylated or methylated, respectively, as compared to the corresponding DNA molecule from one or more reference samples. In the context of cancer, the reference sample may be a normal tissue. Under-methylation of a DNA molecule from a tumor cell means decreased methylation percentage as compared to the normal, e.g., healthy, non-diseased, e.g., non-cancerous, tissue, which is also known as “hypomethylation.” “Hypomethylated” nucleic acid, e.g., cfDNA, fragments can be fragments having a number, e.g., 3 or more, 4 or more, 5 or more, 6 or more, 7 or more, 8 or more, 9 or more, or 10 or more, of CpG sites with a percentage, e.g., 70% or more, 75% or more, 80% or more, 85% or more, 90% or more, or 95% or more, or 97.5% or more, 98% or more, 99% or more, 99.9% or more, of the CpG sites being unmethylated. Over-methylation of a DNA molecule from a tumor cell means increased methylation percentage as compared to the normal e.g., healthy, non-diseased, e.g., non-cancerous, tissue, which is also known as “hypermethylation.” Likewise, “hypermethylated” nucleic acid, e.g., cfDNA, fragments can be fragments having a number, e.g., 3 or more, 4 or more, 5 or more, 6 or more, 7 or more, 8 or more, 9 or more, or 10 or more, of CpG sites with a percentage, e.g., 70% or more, 75% or more, 80% or more, 85% or more, 90% or more, or 95% or more, or 97.5% or more, 98% or more, 99% or more, 99.9% or more of the CpG sites being methylated. “Under-methylated” can also refer to a lower percentage of methylation of a DNA molecule in a target cell as compared to cells of other types, and over-methylated can also refer to a higher percentage of methylation of a DNA molecule in a target cell as compared to cells of other types.

The term “cell free nucleic acid,” refers to nucleic acid, e.g., DNA in “cell free DNA,” and “cfDNA”, fragments that circulate in an individual's body (e.g., bloodstream) and originate from one or more healthy cells and/or from one or more diseased, aged, or damaged cells. Additionally, cell free nucleic acids such as cfDNA may originate from other sources such as viruses, fetuses, etc.

The terms “circulating tumor DNA” and “ctDNA” refer to DNA fragments that originate from tumor cells, which may be released into an individual's bloodstream as result of biological processes such as apoptosis or necrosis of dying cells or actively released by viable tumor cells.

The terms “abnormal methylation pattern” and “anomalous methylation pattern” as used herein refer to a methylation pattern of a nucleic acid, e.g., DNA such as a cfDNA, molecule or a methylation state vector that is found and/or expected to be found in a sample less frequently than it would be in a healthy, e.g., non-cancer, sample. In various embodiments, such a methylation pattern is found and/or expected to be found in a sample with a lower frequency than a value, e.g., a threshold value, of a non-cancer or healthy, e.g., non-cancer, sample. As such, for example, the terms “abnormally methylated” and “anomalously methylated” as used herein describe a nucleic acid, e.g., DNA such as a cfDNA, molecule or a methylation state vector exhibiting an abnormal methylation pattern. An aspect according to the subject disclosure that is differentially methylated can in some versions include an aspect that is abnormally methylated. Also, whether an aspect is differentially methylated can be used as an indicator for a determination of healthy, e.g., non-cancer, as opposed to diseased, e.g., cancer, in referring to the health of a subject from which a subject sample was originated. In some versions, the subject methods include determining whether a nucleic acid, e.g., DNA, molecule or a methylation state vector is abnormally methylated.

The term “methylation state vector” as used herein refers to a vector comprising multiple elements, where each element indicates the methylation status of a methylation site in a nucleic acid, e.g., DNA, molecule including multiple methylation sites, in the order they appear from 5′ to 3′ in the DNA molecule. For example, <Mₓ, Mₓ₊₁, Mₓ₊₂>, <Mₓ, Mₓ₊₁, Uₓ₊₂>, . . . , <Uₓ, Uₓ₊₁, Uₓ₊₂> can be methylation vectors for DNA molecules comprising three methylation sites, where M represents a methylated methylation site and U represents an unmethylated methylation site.

The terms “converted DNA molecules,” and “converted cfDNA molecules,” refer to DNA, e.g., cfDNA, molecules obtained by processing the molecules in a sample for the purpose of differentiating a methylated nucleotide and an unmethylated nucleotide in DNA or cfDNA molecules. For example, in one embodiment, the sample can undergo bisulfite conversion and thus be treated with bisulfite ion (e.g., using sodium bisulfite), to convert unmethylated cytosines (“C”) to uracils (“U”). In another embodiment, the conversion of unmethylated cytosines to uracils is accomplished with enzymatic conversion using an enzymatic conversion reaction, e.g., a reaction using a cytidine deaminase (such as APOBEC). After treatment, converted DNA molecules or cfDNA molecules include additional uracils which are not present in the original cfDNA sample. Replication by DNA polymerase of a DNA strand comprising a uracil results in addition of an adenine to the nascent complementary strand instead of the guanine normally added as the complement to a cytosine or methylcytosine. In some embodiments, the converted DNA molecules are converted hypermethylated DNA molecules.

The term “converted DNA sequence” refers to the sequence of a converted DNA molecule.

The term “tissue of origin” or “TOO” as used herein refers to an organ, organ group, body region and/or cell type that nucleic acid, e.g., cfDNA, such as healthy or disease-associated, e.g., cancer-associated, cfDNA, originates from. The identification of a tissue of origin and/or disease, e.g., cancer, cell type can allow for identification of the most appropriate next steps in a care continuum of a disease to further diagnose, stage and decide on treatment.

Identification of Cell Type Based on DNA Methylation Status

The present disclosure provides compositions and methods for determining cell type based on methylation status of associated DNA fragments. Such DNA fragments typically harbor multiple adjacent CpG dinucleotides having relatively uniform methylation status, methylated or unmethylated, within a cell type. Meanwhile, the methylation status of such CpG sites is different among other cells, thereby enabling the respective cell type(s) to be distinguished from other cell types. Each individual CpG dinucleotide is herein referred to as a “CpG site.” Likewise, a collection of multiple CpG sites within a DNA fragment is referred to as a “CpG cluster.”

Previously, DNA methylation analyses have used primarily bulk tissue, measuring the average methylation for the probed CpG sites, thus precluding the study of minority cell types that may differ in DNA methylation, such as tissue resident immune cells, fibroblasts, or endothelial cells. Alternatively, the analysis of cultured cells often suffers from the inherent limitation of non-physiological methylation patterns introduced in vitro.

To overcome these limitations and to accurately characterize the complexity of the human cell methylome, the instant inventors isolated FACS purified populations of 39 primary human cell types from freshly dissociated adult healthy tissues. Unlike many previous studies which used shallow sequencing or were limited to a subset of genomic regions (reduced representation bisulfite-sequencing, RRBS), this disclosure used deep genome-wide sequencing, with paired-end reads at an average sequencing depth of 32× (±7.2×), in purified human cell populations. For each cell type, the analysis aimed at multiple replicates obtained from different individuals. The analysis coalesced read-specific methylation patterns across the entire genome into larger blocks, allowing simultaneous readout of the methylation status of multiple CpG sites which captured the dependencies between neighboring CpG sites while reflecting the variance of methylation patterns across individual cell types.

As demonstrated in the accompanying experimental examples, surprisingly, in every one of a large number of human cell types examined, a sufficient number of CpG clusters can be identified as having statistically different methylation status between a cell type and all other cell types. Such CpG clusters, also referred to as “methylation markers,” allow identification of each cell type based on its DNA methylation status.

In accordance with one embodiment of the present disclosure, provided are methods for identifying the cell type of the DNA in a biological sample. In some embodiments, the method entails detecting the methylation status of a plurality of CpG sites in a DNA fragment and identifying the corresponding cell type based on the methylation status of the sites. According to various embodiments, the subject DNA fragments are derived from one or more cells of the cell type determined.

### Methylation Detection

Detection of DNA methylation according to the subject embodiments can be carried out with various methods. In some embodiments, the methylation is conversion of a cytosine to a 5-methylcytosine (5-mC). In some embodiments, the methylation is conversion of a cytosine to a 5-hydroxymethylcytosine (5-hmC).

In some embodiments, the methylation status is detected directly, such as with mass spectrometry or methylation-sensitive restriction enzymes. A step of DNA methylation methods can produce converted DNA molecules. In such embodiments, the methylated cytosines are converted prior to further analysis. The terms “convert” and “modify” refer to processing of DNA molecules in a sample for the purpose of differentiating a methylated nucleotide and an unmethylated nucleotide. For example, in one embodiment, the sample can be treated with bisulfite ion (e.g., using sodium bisulfite) to convert unmethylated cytosines (“C”) to uracils (“U”). In another embodiment, the conversion of unmethylated cytosines to uracils is accomplished using an enzymatic conversion reaction, for example, using a cytidine deaminase, such as APOBEC-Seq (NEBiolabs, Ipswich, Mass.). Examples of DNA methylation detection methods are further described below.

Methylation-Specific PCR (MSP), which can be based on a chemical reaction of sodium bisulfite with DNA that converts unmethylated cytosines of CpG dinucleotides to uracil or UpG, followed by traditional PCR. Methylated cytosines will not be converted in this process, and primers are designed to overlap the CpG site of interest, which allows one to determine methylation status as methylated or unmethylated.

Whole genome bisulfite sequencing, also known as BS-Seq, which is a high-throughput genome-wide analysis of DNA methylation. It can also be based on the sodium bisulfite conversion of genomic DNA, which is then sequenced on a Next-Generation Sequencing platform, such as deep sequencing. The sequences obtained are then re-aligned to the reference genome to determine the methylation status of CpG dinucleotides based on mismatches resulting from the conversion of unmethylated cytosines into uracil.

The HpaII tiny fragment Enrichment by Ligation-mediated PCR Assay (HELP Assay) compares representations generated by digestion by a restriction enzyme, e.g., HpaII or MspI, of the genome followed by ligation-mediated PCR. HpaII digests 5′-CCGG-3′ sites when the cytosine in the central CG dinucleotide is unmethylated, the HpaII representation is enriched for the hypomethylated fraction of the genome.

Glal hydrolysis and Ligation Adapter Dependent PCR assay (GLAD-PCR assay) can determine R(5mC)GY sites produced in the course of de novo DNA methylation with DNMT3A and DNMT3B DNA methyltransferases. GLAD-PCR assay do not require bisulfite treatment of the DNA. GLAD-PCR assay uses site-specific methyl-directed DNA-endonucleases (MD DNA endonucleases), which cleave only methylated DNA and do not cleave unmethylated DNA.

The “Illumina Methylation Assay” measures locus-specific DNA methylation using array hybridization. Bisulfite-treated DNA is hybridized to probes on “BeadChips.” Single-base base extension with labeled probes is used to determine methylation status of target sites. The Infinium MethylationEPIC BeadChip can interrogate over 850,000 methylation sites across the human genome.

The “Enzymatic Methyl-seq” or “EM-seq” method developed at New England Biolabs provides an alternative to bisulfite modification. This method relies on the ability of APOBEC (e.g., APOBEC-Seq by NEB) to deaminate cytosines to uracils. Then, cytosines are sequenced as thymines and methylated cytosines are sequenced as cytosines.

### DNA Sample Preparation

DNA fragments subject to the methylation status detection can be prepared from cell-containing or cell-free samples. A biological sample that contains cells can be readily obtained, such as from biopsies, cultured cells, skin tissues, cells, body fluids, without limitation. In some embodiments, a cell-containing biological sample is a tumor tissue or tumor cell. In some embodiments, a cell-containing biological sample is a body fluid sample that contains at least one cell. Non-limiting examples of body fluids that can be implemented according to the subject methods include blood, plasma, serum, semen, milk, urine, vaginal fluid, uterine or vaginal flushing fluids, plural fluid, ascitic fluid, sweat, tears, sputum, bronchoalveolar lavage fluid, stool, saliva and cerebrospinal fluid.

Cell-free DNA samples, in some embodiments, can also be used. Cell-free DNA circulates in an individual's body and may originate from a healthy cell or a diseased, aged, or damaged cell. For a pregnant female, the cell-free DNA may also originate from the fetus. In some embodiments, the cell-free DNA is obtained from a biological sample that includes blood, plasma, serum, semen, milk, urine, saliva or cerebral spinal fluid, or any other body fluid or tissue.

DNA fragments can be isolated from the biological sample with methods known in the art. In some embodiments, the DNA fragments are substantially free of protein, lipids, and other common materials from tissue or fluid samples. In some embodiments, the DNA fragments have suitable length for methylation analysis.

In some embodiments, the DNA fragments have an average length of at least 18, 20, 25, 30, 40, 50, 60, 70, 80, 90, 100, 110, 120, 130, 140, 150, 200, 250, 300, or 350 bp. In some embodiments, the DNA fragments have an average length of not longer than 100, 110, 120, 130, 140, 150, 160, 170, 180, 190, 200, 210, 220, 230, 240, 250, 260, 270, 280, 290, 300 or 350 bp. In some embodiments, the DNA fragments have an average length of 40-300, 400-250, 40-200, 50-300, 50-250, 50-200, 50-150, 100-300, 100-250, 100-200, or 150-300 bp, without limitation.

In some embodiments, the DNA fragments from the biological sample is processed to obtain the desired average lengths. This may be achieved by, for instance, ultrasonic degradation. In some embodiments, the desired average length can be obtained by enriching DNA fragments of the desired lengths while discarding those that are too short or too long, such as by liquid chromatography.

In some embodiments, no degradation of the DNA fragments is needed even if their average lengths are longer than desired. Alternatively, DNA methylation detection can be limited to the desired fragment/sequence with designs of suitable primers (e.g., in methylation-specific PCR) or targeted mapping of detected methylation status within the desired fragment/sequence.

Methylation detection can be performed for the prepared DNA fragments. In some embodiments, it is desirable to detect the methylation status of CpG sites that are adjacent to one another, which collectively form a CpG cluster. The term “adjacent” as used herein, refers to two or more CpG sites all of which are located within region on a DNA fragment. In some embodiments, the region has a length that is not longer than 100, 110, 120, 130, 140, 150, 160, 170, 180, 190, 200, 210, 220, 230, 240, 250, 260, 270, 280, 290, 300, 350, 400, 450 or 500 bp. In some embodiments, a CpG site is considered to be adjacent to another CpG site when their distance is not longer than 100, 110, 120, 130, 140, 150, 160, 170, 180, 190, 200, 210, 220, 230, 240, 250, 260, 270, 280, 290, 300, 350, 400, 450 or 500 bp.

In some embodiments, the methylation status of at least three adjacent CpG sites is detected. In some embodiments, the methylation status of at least four adjacent CpG sites is detected. In some embodiments, the methylation status of at least five adjacent CpG sites is detected. In some embodiments, the methylation status of at least six adjacent CpG sites is detected. In some embodiments, the methylation status of at least seven adjacent CpG sites is detected. In some embodiments, the methylation status of at least eight adjacent CpG sites is detected. In some embodiments, the methylation status of at least nine adjacent CpG sites is detected. In some embodiments, the methylation status of at least ten adjacent CpG sites is detected. In some embodiments, the methylation status of at least 11, 12, 13, 14, or 15 adjacent CpG sites is detected. In some embodiments, the methylation status of at least three, four, five, six, seven, eight, nine, ten, eleven, twelve, thirteen, fourteen, or fifteen CpG sites is detected. Each of such sites can be fully or partially non-adjacent to others. For example, a site can be adjacent to another site on one side and not on the opposite side or can be non-adjacent to other sites on both sides.

### Use of Methylation Markers

The methylation status of these adjacent CpG sites on a DNA fragment can be used according to the subject methods to identify the cell type of the cell from which the DNA fragment originates. In some embodiments, the methylation status of these CpG sites is the frequency of methylated CpG sites, which may be indicated as a percentage (M %). For instance, for DNA fragment F1, which is 200 bp in length and includes 10 CpG sites, its methylation status in a NK cell may be expressed as 20% when two of the CpG sites are methylated and eight of them are not. If the average methylation status of F1 in all other cell types, i.e., cell types that do not include NK cells, ranges from 70% to 90%, then F1 can be a suitable marker for identifying NK cells. For instance, it can be determined according to the subject methods that a cell-free DNA that includes F1 with two of the 10 CpG sites within F1 methylated was released from a NK cell.

Cutoff methylation percentage values, in some embodiments, may be used when determining the cell types. Such cutoff values can be determined based on experimental data such as those presented in the accompanying experimental examples, with suitable statistics and applied according to the subject methods. For instance, if the methylation percentages of F1 in all tested NK cells range from 0-40%, and in all tested non-NK cells range from 60%-100%, then 50% can be applied as a suitable cutoff value. It is to be appreciated that cutoff values are not always required. For instance, when the methylation status of an F1 fragment from an unknown cell is detected and shows 30% methylation, the 30% number can be compared to F1 from NK cell and non-NK cells, and a nearest neighbor can be analyzed and applied to determine the type of the unknown cell.

The methylation status of multiple DNA fragments, in some embodiments, can be used collectively to determine the type of a cell, in a multivariant analysis manner. For instance, when analyzing a cancer cell of unknown primary origin, the methylation status of DNA fragments F1, F2 and F3 can be detected. Methods such as random forest, linear regression, support vector machine, and nearest neighbor, without limitation, can be used to use multiple methylation percentages to determine the primary cell type of the cancer cell.

### Disease Detection and Treatment Monitoring

Cell type identification has important clinical uses. For instance, in many diseases, DNA from dying cells is released into the bloodstream or other body fluids (e.g., semen, milk, urine, saliva and cerebral spinal fluid). Tools that can identify the source tissue of this DNA are useful in identifying and locating diseases. Likewise, a change of the amount of such released DNA can indicate disease progression or treatment effects. For example, the subject methods include measuring an amount of such released DNA at a plurality of time points, such as a first time point and at a second time point later than the first. In some versions, measurements are also taken at a third time point after the second, and/or following consecutive time points. In some versions, a second or additional such time point is after a disease, e.g., cancer, treatment is administered to a subject, e.g., after a resection surgery and/or or therapeutic intervention) and/or a first time point is before such a treatment. The methods can include determining that a disease, e.g., cancer, is worsening or improving based on the difference in DNA amounts between the two or more, e.g., 3 or more, 4 or more, 5 or more, or 10 or more time points. For instance, an increase in an amount of disease, e.g., cancer, DNA can be indicative that the disease, e.g., cancer, condition is worsening whereas a decrease in such DNA can be indicative that the condition is improving. Accordingly, the subject methods can include providing a disease diagnosis and/or treatment protocol based on the determined differences between the plurality of measurements.

Also, for a cancer of unknown primary origin (CUP), the identification of the cell type can help identify its primary origin, which can be key to providing an initial disease diagnosis and/or identifying the suitable treatments.

The subject methods can include detecting such as detecting the tissue(s) of origin of, without limitation: carcinoma, lymphoma, blastoma, sarcoma, and leukemia or lymphoid malignancies. Particular examples of cancers can include, but are not limited to: liver cancer (e.g., hepatocellular carcinoma (FICC)), hepatoma, hepatic carcinoma, bladder cancer (e.g., urothelial bladder cancer), testicular (germ cell tumor) cancer, breast cancer (e.g., HER2 positive, HER2 negative, and triple negative breast cancer), brain cancer (e.g., astrocytoma, glioma (e.g., glioblastoma)), colon cancer, rectal cancer, colorectal cancer, endometrial or uterine carcinoma, salivary gland carcinoma, kidney or renal cancer (e.g., renal cell carcinoma, nephroblastoma or Wilms' tumor), prostate cancer, vulval cancer, squamous cell cancer (e.g., epithelial squamous cell cancer), skin carcinoma, melanoma, lung cancer, including small-cell lung cancer, non-small cell lung cancer (“NSCLC”), adenocarcinoma of the lung and squamous carcinoma of the lung, cancer of the peritoneum, gastric or stomach cancer including gastrointestinal cancer, pancreatic cancer (e.g., pancreatic ductal adenocarcinoma), cervical cancer, ovarian cancer (e.g., high grade serous ovarian carcinoma), thyroid cancer, anal carcinoma, penile carcinoma, head and neck cancer, esophageal carcinoma, and nasopharyngeal carcinoma (NPC). Further examples of cancers include, without limitation: fibrosarcoma, choriocarcinoma, laryngeal carcinomas, retinoblastoma, thecoma, arrhenoblastoma, hematologic malignancies, including but not limited to non-Hodgkin's lymphoma (NHL), multiple myeloma and acute hematologic malignancies, endometriosis, Kaposi's sarcoma, rhabdomyosarcoma, osteogenic sarcoma, leiomyosarcoma, urinary tract carcinomas, Schwannoma, oligodendroglioma, and neuroblastomas.

In some embodiments, cancer according to the subject disclosure can be uterine cancer, upper GI squamous cancer, all other upper GI cancers, thyroid cancer, sarcoma, urothelial renal cancer, all other renal cancers, prostate cancer, pancreatic cancer, ovarian cancer, neuroendocrine cancer, multiple myeloma, melanoma, lymphoma, small cell lung cancer, lung adenocarcinoma, all other lung cancers, leukemia, hepatobiliary carcinoma, hepatobiliary biliary cancer, head and neck cancer, colorectal cancer, cervical cancer, breast cancer, bladder cancer, anorectal cancer, or any combination thereof. Cancer according to the subject embodiments can also be anal cancer, esophageal cancer, head and neck cancer, liver/bile-duct cancer, lung cancer, ovarian cancer, pancreatic cancer, plasma cell neoplasm, stomach cancer, or any combination thereof. Cancer according to the subject embodiments can be thyroid cancer, melanoma, myeloid neoplasm, renal cancer, prostate cancer, breast cancer, uterine cancer, ovarian cancer, bladder cancer, urothelial cancer, cervical cancer, anorectal cancer, head & neck cancer, colorectal cancer, liver cancer, bile duct cancer, pancreatic cancer, gallbladder cancer, upper GI cancer, multiple myeloma, lymphoid neoplasm, lung cancer, or any combination thereof.

Various examples of clinical applications of the present technology are described in further detail below, with respects to example cell types and groups of cell types.

### A. Gastro-Intestinal Cells

The gastro-intestinal (GI) system, or the GI tract, is the tract from the mouth to the anus which includes all the organs of the digestive system in humans and other animals. Food taken in through the mouth is digested to extract nutrients and absorb energy, and the waste expelled as feces. Given their shared functionality, the various different types of cells and tissues in this system share some common molecular, including genetic and epigenetic, characteristics.

### A.1. Oral, Larynx and Esophageal Epithelial Cells

It is discovered herein that some genomic locations are uniformly under-methylated or over-methylated in oral, larynx and esophageal epithelial cells as compared to all other cell types in the human (see, e.g., Table A). For instance, the genomic sequences as provided in SEQ ID NO: 1-15, 16-90, 91-91, 92-101 or 102-125 (annotated with start and end locations on the respective chromosome) all have lower than 40% methylation percentages in oral, larynx or esophageal epithelial cells, and higher than 60% methylation percentages in all other cell types. Likewise, the genomic sequences as provided in SEQ ID NO: 126-133, 134-134 or 135-150 all have relatively higher methylation percentages (>60%) in oral, larynx or esophageal epithelial cells, and lower methylation percentages (<40%) in all other cell types.

TABLE A
Listing of Markers

| | | | SEQ ID NOs | |
|---|---|---|---|---|
| | | | of Markers | |
| Cell type(s) | M/U* | Ranking | From | To |
| Oral, Larynx and Esophageal epithelium | U | Most preferred-top | 1 | 15 |
| Oral, Larynx and Esophageal epithelium | U | Most preferred-extended | 16 | 90 |
| Oral, Larynx and Esophageal epithelium | U | Preferred-extended | 91 | 91 |
| Oral, Larynx and Esophageal epithelium | U | Selected-top | 92 | 101 |
| Oral, Larynx and Esophageal epithelium | U | Selected extended | 102 | 125 |
| Oral, Larynx and Esophageal epithelium | M | Most preferred | 126 | 133 |
| Oral, Larynx and Esophageal epithelium | M | Preferred | 134 | 134 |
| Oral, Larynx and Esophageal epithelium | M | Selected | 135 | 150 |
| Gastric Epithelium | U | Most preferred-top | 151 | 170 |
| Gastric Epithelium | U | Most preferred-extended | 171 | 330 |
| Gastric Epithelium | U | Preferred-extended | 331 | 335 |
| Gastric Epithelium | U | Selected-top | 336 | 340 |
| Gastric Epithelium | U | Selected-extended | 341 | 378 |
| Gastric Epithelium | M | Most preferred | 379 | 401 |
| Gastric Epithelium | M | Preferred | 402 | 402 |
| Gastric Epithelium | M | Selected | 403 | 428 |
| Small Intestine Epithelium | U | Most preferred-top | 429 | 446 |
| Small Intestine Epithelium | U | Most preferred-extended | 447 | 527 |
| Small Intestine Epithelium | U | Preferred-extended | 528 | 529 |
| Small Intestine Epithelium | U | Selected top | 530 | 536 |
| Small Intestine Epithelium | U | Selected-extended | 537 | 554 |
| Small Intestine Epithelium | M | Most preferred | 555 | 564 |
| Small Intestine Epithelium | M | Preferred | 565 | 565 |
| Small Intestine Epithelium | M | Selected | 566 | 579 |
| Colon Epithelium | U | Most preferred-top | 580 | 596 |
| Colon Epithelium | U | Most preferred-extended | 597 | 657 |
| Colon Epithelium | U | Preferred-extended | 658 | 660 |
| Colon Epithelium | U | Selected-top | 661 | 668 |
| Colon Epithelium | U | Selected-extended | 669 | 704 |
| Colon Epithelium | M | Most preferred | 705 | 715 |
| Colon Epithelium | M | Selected | 716 | 729 |
| Colon Fibroblasts | U | Most preferred-top | 730 | 732 |
| Colon Fibroblasts | M | Most preferred | 733 | 739 |
| Colon Fibroblasts | M | Selected | 740 | 741 |
| Gallbladder Epithelium | U | Most preferred-top | 742 | 758 |
| Gallbladder Epithelium | U | Most preferred-extended | 759 | 829 |
| Gallbladder Epithelium | U | Preferred-extended | 830 | 831 |
| Gallbladder Epithelium | U | Selected-top | 832 | 839 |
| Gallbladder Epithelium | U | Selected-extended | 840 | 867 |
| Gallbladder Epithelium | M | Most preferred | 868 | 875 |
| Gallbladder Epithelium | M | Selected | 876 | 876 |
| Liver Hepatocytes | U | Most preferred-top | 877 | 896 |
| Liver Hepatocytes | U | Most preferred-extended | 897 | 980 |
| Liver Hepatocytes | U | Preferred-top | 981 | 983 |
| Liver Hepatocytes | U | Preferred-extended | 984 | 986 |
| Liver Hepatocytes | U | Selected-top | 987 | 988 |
| Liver Hepatocytes | U | Selected-extended | 989 | 1002 |
| Liver Hepatocytes | M | Most preferred | 1003 | 1018 |
| Liver Hepatocytes | M | Preferred | 1019 | 1023 |
| Liver Hepatocytes | M | Selected | 1024 | 1027 |
| Pancreatic Acinar cells | U | Most preferred-top | 1028 | 1041 |
| Pancreatic Acinar cells | U | Most preferred-extended | 1042 | 1112 |
| Pancreatic Acinar cells | U | Preferred-extended | 1113 | 1116 |
| Pancreatic Acinar cells | U | Selected-top | 1117 | 1127 |
| Pancreatic Acinar cells | U | Selected-extended | 1128 | 1155 |
| Pancreatic Acinar cells | M | Most preferred | 1156 | 1161 |
| Pancreatic Acinar cells | M | Selected | 1162 | 1180 |
| Pancreatic Alpha cells | U | Most preferred-top | 1181 | 1198 |
| Pancreatic Alpha cells | U | Most preferred-extended | 1199 | 1282 |
| Pancreatic Alpha cells | U | Preferred-top | 1283 | 1284 |
| Pancreatic Alpha cells | U | Preferred-extended | 1285 | 1287 |
| Pancreatic Alpha cells | U | Selected-top | 1288 | 1292 |
| Pancreatic Alpha cells | U | Selected-extended | 1293 | 1306 |
| Pancreatic Alpha cells | M | Most preferred | 1307 | 1315 |
| Pancreatic Alpha cells | M | Preferred | 1316 | 1316 |
| Pancreatic Alpha cells | M | Selected | 1317 | 1331 |
| Pancreatic Beta cells | U | Most preferred-top | 1332 | 1351 |
| Pancreatic Beta cells | U | Most preferred-extended | 1352 | 1440 |
| Pancreatic Beta cells | U | Selected-top | 1441 | 1445 |
| Pancreatic Beta cells | U | Selected-extended | 1446 | 1460 |
| Pancreatic Beta cells | M | Most preferred | 1461 | 1471 |
| Pancreatic Beta cells | M | Selected | 1472 | 1485 |
| Pancreatic Delta cells | U | Most preferred-top | 1486 | 1508 |
| Pancreatic Delta cells | U | Most preferred-extended | 1509 | 1594 |
| Pancreatic Delta cells | U | Preferred-extended | 1595 | 1596 |
| Pancreatic Delta cells | U | Selected-top | 1597 | 1598 |
| Pancreatic Delta cells | U | Selected-extended | 1599 | 1613 |
| Pancreatic Delta cells | M | Most preferred | 1614 | 1624 |
| Pancreatic Delta cells | M | Preferred | 1625 | 1625 |
| Pancreatic Delta cells | M | Selected | 1626 | 1638 |
| Pancreatic Ductal cells | U | Most preferred-top | 1639 | 1658 |
| Pancreatic Ductal cells | U | Most preferred-extended | 1659 | 1742 |
| Pancreatic Ductal cells | U | Preferred-top | 1743 | 1743 |
| Pancreatic Ductal cells | U | Preferred-extended | 1744 | 1747 |
| Pancreatic Ductal cells | U | Selected-top | 1748 | 1751 |
| Pancreatic Ductal cells | U | Selected-extended | 1752 | 1767 |
| Pancreatic Ductal cells | M | Most preferred | 1768 | 1779 |
| Pancreatic Ductal cells | M | Selected | 1780 | 1792 |
| Endometrium Epithelium | U | Most preferred-extended | 1793 | 1864 |
| Endometrium Epithelium | U | Preferred-extended | 1865 | 1872 |
| Endometrium Epithelium | U | Selected-extended | 1873 | 1892 |
| Endometrium Epithelium | M | Most preferred | 1893 | 1905 |
| Endometrium Epithelium | M | Selected | 1906 | 1917 |
| Fallopian Epithelium | U | Most preferred-top | 1918 | 1937 |
| Fallopian Epithelium | U | Most preferred-extended | 1938 | 2022 |
| Fallopian Epithelium | U | Preferred-extended | 2023 | 2024 |
| Fallopian Epithelium | U | Selected-top | 2025 | 2029 |
| Fallopian Epithelium | U | Selected-extended | 2030 | 2042 |
| Fallopian Epithelium | M | Most preferred | 2043 | 2061 |
| Fallopian Epithelium | M | Selected | 2062 | 2067 |
| Kidney Epithelium | U | Most preferred-top | 2068 | 2080 |
| Kidney Epithelium | U | Most preferred-extended | 2081 | 2141 |
| Kidney Epithelium | U | Preferred-extended | 2142 | 2144 |
| Kidney Epithelium | U | Selected-top | 2145 | 2156 |
| Kidney Epithelium | U | Selected-extended | 2157 | 2194 |
| Kidney Epithelium | M | Most preferred | 2195 | 2209 |
| Kidney Epithelium | M | Selected | 2210 | 2219 |
| Bladder Epithelium | U | Most preferred-top | 2220 | 2233 |
| Bladder Epithelium | U | Most preferred-extended | 2234 | 2298 |
| Bladder Epithelium | U | Preferred-top | 2299 | 2299 |
| Bladder Epithelium | U | Preferred-extended | 2300 | 2303 |
| Bladder Epithelium | U | Selected-top | 2304 | 2313 |
| Bladder Epithelium | U | Selected-extended | 2314 | 2345 |
| Bladder Epithelium | M | Most preferred | 2346 | 2350 |
| Bladder Epithelium | M | Preferred | 2351 | 2351 |
| Bladder Epithelium | M | Selected | 2352 | 2370 |
| Prostate Epithelium | U | Most preferred-top | 2371 | 2389 |
| Prostate Epithelium | U | Most preferred-extended | 2390 | 2476 |
| Prostate Epithelium | U | Preferred-extended | 2477 | 2480 |
| Prostate Epithelium | U | Selected top | 2481 | 2486 |
| Prostate Epithelium | U | Selected-extended | 2487 | 2495 |
| Prostate Epithelium | M | Most preferred | 2496 | 2500 |
| Prostate Epithelium | M | Preferred | 2501 | 2501 |
| Prostate Epithelium | M | Selected | 2502 | 2520 |
| Breast Basal Epithelium | U | Most preferred-top | 2521 | 2536 |
| Breast Basal Epithelium | U | Most preferred-extended | 2537 | 2616 |
| Breast Basal Epithelium | U | Selected-top | 2617 | 2625 |
| Breast Basal Epithelium | U | Selected-extended | 2626 | 2651 |
| Breast Basal Epithelium | M | Most preferred | 2652 | 2659 |
| Breast Basal Epithelium | M | Selected | 2660 | 2676 |
| Breast Luminal Epithelium | U | Most preferred-top | 2677 | 2688 |
| Breast Luminal Epithelium | U | Most preferred-extended | 2689 | 2748 |
| Breast Luminal Epithelium | U | Preferred-extended | 2749 | 2749 |
| Breast Luminal Epithelium | U | Selected top | 2750 | 2762 |
| Breast Luminal Epithelium | U | Selected-extended | 2763 | 2802 |
| Breast Luminal Epithelium | M | Most preferred | 2803 | 2815 |
| Breast Luminal Epithelium | M | Preferred | 2816 | 2816 |
| Breast Luminal Epithelium | M | Selected | 2817 | 2827 |
| Lung Alveolar Epithelium | U | Most preferred-top | 2828 | 2838 |
| Lung Alveolar Epithelium | U | Most preferred-extended | 2839 | 2899 |
| Lung Alveolar Epithelium | U | Preferred-top | 2900 | 2900 |
| Lung Alveolar Epithelium | U | Preferred-extended | 2901 | 2903 |
| Lung Alveolar Epithelium | U | Selected-top | 2904 | 2916 |
| Lung Alveolar Epithelium | U | Selected-extended | 2917 | 2953 |
| Lung Alveolar Epithelium | M | Most preferred | 2954 | 2960 |
| Lung Alveolar Epithelium | M | Selected | 2961 | 2978 |
| Lung Bronchial Epithelium | U | Most preferred-top | 2979 | 3001 |
| Lung Bronchial Epithelium | U | Most preferred-extended | 3002 | 3087 |
| Lung Bronchial Epithelium | U | Preferred-extended | 3088 | 3090 |
| Lung Bronchial Epithelium | U | Selected-top | 3091 | 3092 |
| Lung Bronchial Epithelium | U | Selected-extended | 3093 | 3104 |
| Lung Bronchial Epithelium | M | Most preferred | 3105 | 3109 |
| Lung Bronchial Epithelium | M | Selected | 3110 | 3129 |
| Heart Cardiomyocytes | U | Most preferred-top | 3130 | 3147 |
| Heart Cardiomyocytes | U | Most preferred-extended | 3148 | 3223 |
| Heart Cardiomyocytes | U | Selected-top | 3224 | 3230 |
| Heart Cardiomyocytes | U | Selected-extended | 3231 | 3254 |
| Heart Cardiomyocytes | M | Most preferred | 3255 | 3266 |
| Heart Cardiomyocytes | M | Preferred | 3267 | 3267 |
| Heart Cardiomyocytes | M | Selected | 3268 | 3279 |
| Heart Fibroblasts | U | Most preferred-top | 3280 | 3300 |
| Heart Fibroblasts | U | Most preferred-extended | 3301 | 3394 |
| Heart Fibroblasts | U | Preferred-extended | 3395 | 3396 |
| Heart Fibroblasts | U | Selected-top | 3397 | 3400 |
| Heart Fibroblasts | U | Selected-extended | 3401 | 3407 |
| Heart Fibroblasts | M | Most preferred | 3408 | 3414 |
| Heart Fibroblasts | M | Preferred | 3415 | 3416 |
| Heart Fibroblasts | M | Selected | 3417 | 3432 |
| Vascular Endothelial cells | U | Most preferred-top | 3433 | 3456 |
| Vascular Endothelial cells | U | Most preferred-extended | 3457 | 3547 |
| Vascular Endothelial cells | U | Preferred-extended | 3548 | 3550 |
| Vascular Endothelial cells | U | Selected-top | 3551 | 3551 |
| Vascular Endothelial cells | U | Selected-extended | 3552 | 3559 |
| Vascular Endothelial cells | M | Most preferred | 3560 | 3579 |
| Vascular Endothelial cells | M | Preferred | 3580 | 3580 |
| Vascular Endothelial cells | M | Selected | 3581 | 3584 |
| Blood B cells | U | Most preferred-top | 3585 | 3607 |
| Blood B cells | U | Most preferred-extended | 3608 | 3701 |
| Blood B cells | U | Preferred-extended | 3702 | 3702 |
| Blood B cells | U | Selected-top | 3703 | 3704 |
| Blood B cells | U | Selected-extended | 3705 | 3712 |
| Blood B cells | M | Most preferred | 3713 | 3733 |
| Blood B cells | M | Selected | 3734 | 3737 |
| Blood Granulocytes | U | Most preferred-top | 3738 | 3758 |
| Blood Granulocytes | U | Most preferred-extended | 3759 | 3849 |
| Blood Granulocytes | U | Preferred-extended | 3850 | 3851 |
| Blood Granulocytes | U | Selected-top | 3852 | 3855 |
| Blood Granulocytes | U | Selected extended | 3856 | 3862 |
| Blood Granulocytes | M | Most preferred | 3863 | 3884 |
| Blood Granulocytes | M | Preferred | 3885 | 3885 |
| Blood Granulocytes | M | Selected | 3886 | 3886 |
| Blood Monocytes + Macrophages | U | Most preferred-top | 3887 | 3909 |
| Blood Monocytes + Macrophages | U | Most preferred-extended | 3910 | 3997 |
| Blood Monocytes + Macrophages | U | Preferred-extended | 3998 | 4000 |
| Blood Monocytes + Macrophages | U | Selected-top | 4001 | 4002 |
| Blood Monocytes + Macrophages | U | Selected-extended | 4003 | 4012 |
| Blood Monocytes + Macrophages | M | Most preferred | 4013 | 4036 |
| Blood Monocytes + Macrophages | M | Selected | 4037 | 4037 |
| Blood NK cells | U | Most preferred-top | 4038 | 4061 |
| Blood NK cells | U | Most preferred-extended | 4062 | 4146 |
| Blood NK cells | U | Preferred-extended | 4147 | 4148 |
| Blood NK cells | U | Selected top | 4149 | 4149 |
| Blood NK cells | U | Selected-extended | 4150 | 4162 |
| Blood NK cells | M | Most preferred | 4163 | 4184 |
| Blood NK cells | M | Selected | 4185 | 4187 |
| Blood T cells | U | Most preferred-top | 4188 | 4205 |
| Blood T cells | U | Most preferred-extended | 4206 | 4274 |
| Blood T cells | U | Preferred-top | 4275 | 4275 |
| Blood T cells | U | Preferred-extended | 4276 | 4276 |
| Blood T cells | U | Selected-top | 4277 | 4282 |
| Blood T cells | U | Selected-extended | 4283 | 4312 |
| Blood T cells | M | Most preferred | 4313 | 4322 |
| Blood T cells | M | Preferred | 4323 | 4323 |
| Blood T cells | M | Selected | 4324 | 4337 |
| Erythrocyte progenitor cells | U | Most preferred-top | 4338 | 4361 |
| Erythrocyte progenitor cells | U | Most preferred-extended | 4362 | 4449 |
| Erythrocyte progenitor cells | U | Preferred-extended | 4450 | 4453 |
| Erythrocyte progenitor cells | U | Selected-top | 4454 | 4454 |
| Erythrocyte progenitor cells | U | Selected-extended | 4455 | 4464 |
| Erythrocyte progenitor cells | M | Most preferred | 4465 | 4470 |
| Epidermal Keratinocytes | U | Most preferred-top | 4471 | 4492 |
| Epidermal Keratinocytes | U | Most preferred-extended | 4493 | 4573 |
| Epidermal Keratinocytes | U | Preferred-top | 4574 | 4574 |
| Epidermal Keratinocytes | U | Preferred-extended | 4575 | 4577 |
| Epidermal Keratinocytes | U | Selected-top | 4578 | 4579 |
| Epidermal Keratinocytes | U | Selected-extended | 4580 | 4595 |
| Epidermal Keratinocytes | M | Most preferred | 4596 | 4598 |
| Epidermal Keratinocytes | M | Preferred | 4599 | 4599 |
| Epidermal Keratinocytes | M | Selected | 4600 | 4618 |
| Dermal Fibroblasts | U | Most preferred-top | 4619 | 4641 |
| Dermal Fibroblasts | U | Most preferred-extended | 4642 | 4719 |
| Dermal Fibroblasts | U | Preferred-top | 4720 | 4720 |
| Dermal Fibroblasts | U | Preferred-extended | 4721 | 4727 |
| Dermal Fibroblasts | U | Selected-top | 4728 | 4728 |
| Dermal Fibroblasts | U | Selected-extended | 4729 | 4741 |
| Dermal Fibroblasts | M | Most preferred | 4742 | 4747 |
| Dermal Fibroblasts | M | Preferred | 4748 | 4748 |
| Dermal Fibroblasts | M | Selected | 4749 | 4766 |
| Osteoblasts | U | Most preferred-top | 4767 | 4783 |
| Osteoblasts | U | Most preferred-extended | 4784 | 4869 |
| Osteoblasts | U | Preferred-top | 4870 | 4872 |
| Osteoblasts | U | Preferred-extended | 4873 | 4877 |
| Osteoblasts | U | Selected top | 4878 | 4882 |
| Osteoblasts | U | Selected-extended | 4883 | 4891 |
| Osteoblasts | M | Most preferred | 4892 | 4897 |
| Osteoblasts | M | Selected | 4898 | 4916 |
| Skeletal Muscle cells | U | Most preferred-top | 4917 | 4937 |
| Skeletal Muscle cells | U | Most preferred-extended | 4938 | 5016 |
| Skeletal Muscle cells | U | Preferred-top | 5017 | 5017 |
| Skeletal Muscle cells | U | Preferred-extended | 5018 | 5023 |
| Skeletal Muscle cells | U | Selected-top | 5024 | 5026 |
| Skeletal Muscle cells | U | Selected-extended | 5027 | 5040 |
| Skeletal Muscle cells | M | Most preferred | 5041 | 5043 |
| Skeletal Muscle cells | M | Preferred | 5044 | 5045 |
| Skeletal Muscle cells | M | Selected | 5046 | 5064 |
| Smooth Muscle cells | U | Most preferred-top | 5065 | 5086 |
| Smooth Muscle cells | U | Most preferred-extended | 5087 | 5178 |
| Smooth Muscle cells | U | Preferred-top | 5179 | 5179 |
| Smooth Muscle cells | U | Preferred-extended | 5180 | 5181 |
| Smooth Muscle cells | U | Selected-top | 5182 | 5183 |
| Smooth Muscle cells | U | Selected-extended | 5184 | 5191 |
| Smooth Muscle cells | M | Most preferred | 5192 | 5204 |
| Smooth Muscle cells | M | Preferred | 5205 | 5207 |
| Smooth Muscle cells | M | Selected | 5208 | 5216 |
| Thyroid Epithelium | U | Most preferred-top | 5217 | 5230 |
| Thyroid Epithelium | U | Most preferred-extended | 5231 | 5284 |
| Thyroid Epithelium | U | Preferred-extended | 5285 | 5285 |
| Thyroid Epithelium | U | Selected-top | 5286 | 5296 |
| Thyroid Epithelium | U | Selected-extended | 5297 | 5343 |
| Thyroid Epithelium | M | Most preferred | 5344 | 5358 |
| Thyroid Epithelium | M | Preferred | 5359 | 5359 |
| Thyroid Epithelium | M | Selected | 5360 | 5368 |
| Adipocytes | U | Most preferred-top | 5369 | 5389 |
| Adipocytes | U | Most preferred-extended | 5390 | 5445 |
| Adipocytes | U | Preferred-top | 5446 | 5446 |
| Adipocytes | U | Selected-top | 5447 | 5449 |
| Adipocytes | U | Selected-extended | 5450 | 5453 |
| Adipocytes | M | Most preferred | 5454 | 5463 |
| Adipocytes | M | Preferred | 5464 | 5464 |
| Adipocytes | M | Selected | 5465 | 5470 |
| Neuron CNS | U | Most preferred-top | 5471 | 5488 |
| Neuron CNS | U | Most preferred-extended | 5489 | 5556 |
| Neuron CNS | U | Preferred-extended | 5557 | 5559 |
| Neuron CNS | U | Selected top | 5560 | 5566 |
| Neuron CNS | U | Selected-extended | 5567 | 5594 |
| Neuron CNS | M | Most preferred | 5595 | 5613 |
| Neuron CNS | M | Selected | 5614 | 5619 |
| Oligodendrocytes | U | Most preferred-top | 5620 | 5649 |
| Oligodendrocytes | U | Most preferred-extended | 5650 | 5721 |
| Oligodendrocytes | U | Preferred-extended | 5722 | 5724 |
| Oligodendrocytes | U | Selected-top | 5725 | 5744 |
| Oligodendrocytes | U | Selected-extended | 5745 | 5771 |
| Oligodendrocytes | M | Most preferred | 5772 | 5782 |
| Oligodendrocytes | M | Preferred | 5783 | 5783 |
| Oligodendrocytes | M | Selected | 5784 | 5796 |
| Neurons + Oligodendrocytes | U | Most preferred-extended | 5797 | 5870 |
| Neurons + Oligodendrocytes | U | Selected extended | 5871 | 5898 |
| Neurons + Oligodendrocytes | M | Most preferred | 5899 | 5911 |
| Neurons + Oligodendrocytes | M | Preferred | 5912 | 5912 |
| Neurons + Oligodendrocytes | M | Selected | 5913 | 5923 |
| Pancreatic Alpha + Beta + Delta cells | U | Most preferred-top | 5924 | 5935 |
| Pancreatic Alpha + Beta + Delta cells | U | Most preferred-extended | 5936 | 6011 |
| Pancreatic Alpha + Beta + Delta cells | U | Preferred-top | 6012. | 6012 |
| Pancreatic Alpha + Beta + Delta cells | U | Preferred-extended | 6013 | 6014 |
| Pancreatic Alpha + Beta + Delta cells | U | Selected-top | 6015 | 6026 |
| Pancreatic Alpha + Beta + Delta cells | U | Selected-extended | 6027 | 6050 |
| Pancreatic Alpha + Beta + Deita cells | M | Most preferred | 6051 | 6057 |
| Pancreatic Alpha + Beta + Delta cells | M | Selected | 6058 | 6075 |
| Breast Basal + Luminal Epithelium | U | Most preferred-top | 6076 | 6090 |
| Breast Basal + Luminal Epithelium | U | Most preferred-extended | 6091 | 6159 |
| Breast Basal + Luminal Epithelium | U | Preferred-top | 6160 | 6160 |
| Breast Basal + Luminal Epithelium | U | Preferred-extended | 6161 | 6162 |
| Breast Basal + Luminal Epithelium | U | Selected-top | 6163 | 6171 |
| Breast Basal + Luminal Epithelium | U | Selected-extended | 6172 | 6201 |
| Breast Basal + Luminal Epithelium | M | Most preferred | 6202 | 6206 |
| Breast Basal + Luminal Epithelium | M | Selected | 6207 | 6226 |
| Lung Alveolar + Bronchial cells | U | Most preferred-top | 6227 | 6243 |
| Lung Alveolar + Bronchial cells | U | Most preferred-extended | 6244 | 6326 |
| Lung Alveolar + Bronchial cells | U | Preferred-top | 6327 | 6327 |
| Lung Alveolar + Bronchial cells | U | Preferred-extended | 6328 | 6329 |
| Lung Alveolar + Bronchial cells | U | Selected-top | 6330 | 6336 |
| Lung Alveolar + Bronchial cells | U | Selected-extended | 6337 | 6352 |
| Lung Alveolar + Bronchial cells | M | Most preferred | 6353 | 6353 |
| Lung Alveolar + Bronchial cells | M | Selected | 6354 | 6365 |
| Fallopian + Ovary Epithelium | U | Most preferred-top | 6366 | 6399 |
| Fallopian + Ovary Epithelium | U | Most preferred-extended | 6400 | 6468 |
| Fallopian + Ovary Epithelium | U | Preferred-extended | 6469 | 6475 |
| Fallopian + Ovary Epithelium | U | Selected-top | 6476 | 6491 |
| Fallopian + Ovary Epithelium | U | Selected-extended | 6492 | 6515 |
| Fallopian + Ovary Epithelium | M | Most preferred | 6516 | 6527 |
| Fallopian + Ovary Epithelium | M | Selected | 6528 | 6540 |
| Gastric + Small Intes. + Colon Epithelium | U | Most preferred-top | 6541 | 6556 |
| Gastric + Small Intes. + Colon Epithelium | U | Preferred-top | 6557 | 6557 |
| Gastric + Small Intes. + Colon Epithelium | U | Selected-top | 6558 | 6565 |
| Gastric + Small Intes. Epithelium | U | Most preferred-top | 6566 | 6589 |
| Gastric + Small Intes. Epithelium | U | Most preferred-extended | 6590 | 6672 |
| Gastric + Small Intes. Epithelium | U | Preferred-extended | 6673 | 6673 |
| Gastric + Small Intes. Epithelium | U | Selected top | 6674 | 6674 |
| Gastric + Small Intes. Epithelium | U | Selected extended | 6675 | 6690 |
| Gastric + Small Intes. Epithelium | M | Preferred | 6691 | 6691 |
| Gastric + Small Intes. Epithelium | M | Selected | 6692 | 6694 |
| Small Intes. + Colon Epithelium | U | Most preferred-top | 6695 | 6702 |
| Small Intes. + Colon Epithelium | U | Most preferred-extended | 6703 | 6760 |
| Small Intes. + Colon Epithelium | U | Selected-top | 6761 | 6777 |
| Small Intes. + Colon Epithelium | U | Selected-extended | 6778 | 6820 |
| Small Intes. + Colon Epithelium | M | Most preferred | 6821 | 6825 |
| Small Intes. + Colon Epithelium | M | Selected | 6826 | 6845 |
| Colon + Heart Fibroblasts | U | Most preferred-top | 6846 | 6863 |
| Colon + Heart Fibroblasts | U | Most preferred-extended | 6864 | 6869 |
| Colon + Heart Fibroblasts | U | Preferred-top | 6870 | 6872 |
| Colon + Heart Fibroblasts | U | Selected-top | 6873 | 6876 |
| Colon + Heart Fibroblasts | U | Selected extended | 6877 | 6878 |
| Colon + Heart Fibroblasts | M | Most preferred | 6879 | 6890 |
| Colon + Heart Fibroblasts | M | Selected | 6891 | 6898 |
| Cardiomyocytes + Skeletal + Smooth | U | Most preferred-top | 6899 | 6906 |
| muscle cells | | | | |
| Cardiomyocytes + Skeletal + Smooth | U | Most preferred-extended | 6907 | 6907 |
| muscle cells | | | | |
| Cardiomyocytes + Skeletal + Smooth | U | Selected-top | 6908 | 6909 |
| muscle cells | | | | |
| Cardiomyocytes + Skeletal + Smooth | M | Most preferred | 6910 | 6911 |
| muscle cells | | | | |
| Skeletal + Smooth muscle cells | U | Most preferred-top | 6912 | 6929 |
| Skeletal + Smooth muscle cells | U | Most preferred-extended | 6930 | 6930 |
| Skeletal + Smooth muscle cells | U | Selected-top | 6931 | 6931 |
| Skeletal + Smooth muscle cells | M | Most preferred | 6932 | 6936 |
| Skeletal + Smooth muscle cells | M | Selected | 6937 | 6939 |
| Heart Cardiomyocytes + Fibroblasts | U | Most preferred-top | 6940 | 6959 |
| Heart Cardiomyocytes + Fibroblasts | U | Most preferred-extended | 6960 | 7045 |
| Heart Cardiomyocytes + Fibroblasts | U | Preferred-top | 7046 | 7046 |
| Heart Cardiomyocytes + Fibroblasts | U | Preferred-extended | 7047 | 7049 |
| Heart Cardiomyocytes + Fibroblasts | U | Selected-top | 7050 | 7053 |
| Heart Cardiomyocytes + Fibroblasts | U | Selected-extended | 7054 | 7065 |
| Heart Cardiomyocytes + Fibroblasts | M | Most preferred | 7066 | 7082 |
| Heart Cardiomyocytes + Fibroblasts | M | Selected | 7083 | 7090 |

*U: lower methylation (unmethylated) in the specific cell type and higher methylation in oilier cell types;
M: higher methylation (methylated) in the specific cell type and lower methylation in other cell types.

Each genomic sequence in the sequence listing (according to the human genome version hg19, Genome Reference Consortium Human Build 37 (GRCh37), published Feb. 27, 2009) represents DNA fragments that includes or overlaps with the genomic sequence. In some embodiments, a DNA fragment that includes a CpG cluster which can be used as methylation marker, includes at least a CpG site contained in a genomic sequence as defined in the sequence listing. In some embodiments, the DNA fragment includes at least two, three, four, five, six, seven, eight, nine, ten or more CpG sites contained in a genomic sequence as defined in the sequence listing.

The Sequence Listing is concurrently submitted in ASCII format and is hereby incorporated by reference in its entirety. A listing of all sequences, without the actual sequences, is provided in Table B. Each sequence (see example shown in Table C) is annotated with respect to its genomic location (e.g., chr9:119238427-119238709), nearby gene and location (e.g., intron of ASTN2) and region, the corresponding cell type (e.g., Oral, Larynx and Esophageal epithelium), whether it is under-methylated (U) or over-methylated (M) in the corresponding cell type, and average methylation frequency within the cell type versus all other cell types (e.g., 0.05:0.94).

TABLE B
Target Sequences

| SEQ ID NO: | Chr | start | end | Cell | W/I | Out | Genomic class | Gene | U/M |
|---|---|---|---|---|---|---|---|---|---|
| 1 | chr1 | 2323726581 | 232372668 | Oral, Larynx and Esophageal epithelium | 0.05 | 0.94 | Intergenic | SIPA1L2 | U |
| 2 | chr9 | 119238427 | 119238709 | Oral, Larynx and Esophageal epithelium | 0.05 | 0.94 | intron | ASTN2 | U |
| 3 | chr22 | 45557574 | 45557798 | Oral, Larynx and Esophageal epithelium | 0.03 | 0.91 | intron | LOC100506714 | U |
| 4 | chr4 | 69018630 | 69018724 | Oral, Larynx and Esophageal epithelium | 0.05 | 0.9 | Intergenic | TMPRSS11F | U |
| 5 | chr12 | 53251350 | 53251751 | Oral, Larynx and Esophageal epithelium | 0.07 | 0.9 | Intergenic | KRT78 | U |
| 6 | chr1 | 550960653 | 55096321 | Oral, Larynx and Esophageal epithelium | 0.08 | 0.88 | exon, intron | ACOT11 | U |
| 7 | chr7 | 110559652 | 110559954 | Oral, Larynx and Esophageal epithelium | 0.12 | 0.91 | intron | IMMP2L | U |
| 8 | chr14 | 99419301 | 99419516 | Oral, Larynx and Esophageal epithelium | 0.04 | 0.83 | Intergenic | C14orf177 | U |
| 9 | chr13 | 68460537 | 68460622 | Oral, Larynx and Esophageal epithelium | 0.1 | 0.88 | Intergenic | PCDH9 | U |
| 10 | chr10 | 124095969 | 124096178 | Oral, Larynx and Esophageal epithelium | 0.13 | 0.91 | exon | BTBD16 | U |
| 11 | chr1 | 249138696 | 249138854 | Oral, Larynx and Esophageal epithelium | 0.16 | 0.94 | exon | ZNF672 | U |
| 12 | chr11 | 128518761 | 128519110 | Oral, Larynx and Esophageal epithelium | 0.11 | 0.88 | Intergenic | FLI1 | U |
| 13 | chr5 | 92022171 | 9202323 | Oral, Larynx and Esophageal epithelium | 0.11 | 0.86 | exon | SEMA5A | U |
| 14 | chr11 | 130242997 | 130243057 | Oral, Larynx and Esophageal epithelium | 0.23 | 0.93 | Intergenic | ADAMTS8 | U |
| 15 | chr11 | 65680975 | 65681143 | Oral, Larynx and Esophageal epithelium | 0.21 | 0.87 | Intergenic | C11orf68 | U |
| 16 | chr22 | 45526552 | 45526623 | Oral, Larynx and Esophageal epithelium | 0.09 | 0.93 | Intergenic | LOC100506714 | U |
| 17 | chr1 | 156682901 | 156683132 | Oral, Larynx and Esophageal epithelium | 0.04 | 0.88 | Intergenic | CRABP2 | U |
| 18 | chr5 | 125932585 | 125932791 | Oral, Larynx and Esophageal epithelium | 0.08 | 0.89 | Intergenic | ALDH7A1 | U |
| 19 | chr18 | 9012779 | 9013019 | Oral, Larynx and Esophageal epithelium | 0.06 | 0.87 | Intergenic | NDUFV2 | U |
| 20 | chr3 | 1266507061 | 126650821 | Oral, Larynx and Esophageal epithelium | 0.13 | 0.93 | intron | CHCHD6 | U |
| 21 | chr15 | 43589375 | 43589552 | Oral, Larynx and Esophageal epithelium | 0.08 | 0.88 | intron | TGM7 | U |
| 22 | chr17 | 70698846 | 70699033 | Oral, Larynx and Esophageal epithelium | 0.08 | 0.88 | intron | SLC39A11 | U |
| 23 | chr15 | 67728831 | 67729070 | Oral, Larynx and Esophageal epithelium | 0.11 | 0.91 | intron | IQCH-AS1 | U |
| 24 | chr3 | 24511875 | 24512177 | Oral, Larynx and Esophageal epithelium | 0.04 | 0.84 | intron | THRB | U |
| 25 | chr3 | 591467741 | 59147199 | Oral, Larynx and Esophageal epithelium | 0.08 | 0.88 | Intergenic | C3orf67 | U |
| 26 | chr19 | 51862398 | 51862855 | Oral, Larynx and Esophageal epithelium | 0.09 | 0.89 | intron | ETFB | U |
| 27 | chr15 | 89035700 | 89035877 | Oral, Larynx and Esophageal epithelium | 0.07 | 0.86 | Intergenic | MRPS11 | U |
| 28 | chr20 | 49053090 | 49053397 | Oral, Larynx and Esophageal epithelium | 0.08 | 0.87 | Intergenic | PTPN1 | U |
| 29 | chr15 | 44197814 | 44198149 | Oral, Larynx and Esophageal epithelium | 0.13 | 0.92 | exon, intron | FRMD5, FRMD5 | U |
| 30 | chr2 | 17623091 | 17623243 | Oral, Larynx and Esophageal epithelium | 0.12 | 0.9 | Intergenic | RAD51AP2 | U |
| 31 | chr8 | 365545681 | 36554809 | Oral, Larynx and Esophageal epithelium | 0.04 | 0.82 | Intergenic | KCNU1 | U |
| 32 | chr16 | 84438956 | 84439252 | Oral, Larynx and Esophageal epithelium | 0.09 | 0.87 | intron | ATP2C2 | U |
| 33 | chr10 | 1160741173 | 116074458 | Oral, Larynx and Esophageal epithelium | 0.09 | 0.87 | intron | AFAP1L2 | U |
| 34 | chr12 | 31831608 | 31832041 | Oral, Larynx and Esophageal epithelium | 0.11 | 0.89 | intron | AMN1 | U |
| 35 | chr9 | 126114492 | 126114599 | Oral, Larynx and Esophageal epithelium | 0.08 | 0.84 | Intergenic | CRB2 | U |
| 36 | chr3 | 126650914 | 126651054 | Oral, Larynx and Esophageal epithelium | 0.13 | 0.89 | intron | CHCHD6 | U |
| 37 | chr15 | 43388602 | 43388802 | Oral, Larynx and Esophageal epithelium | 0.17 | 0.93 | intron | UBR1 | U |
| 38 | chr13 | 101197929 | 101198167 | Oral, Larynx and Esophageal epithelium | 0.13 | 0.89 | intron | GGACT | U |
| 39 | chr1 | 23583336 | 23583588 | Oral, Larynx and Esophageal epithelium | 0.15 | 0.91 | Intergenic | HTR1D | U |
| 40 | chr1 | 45542837 | 45543022 | Oral, Larynx and Esophageal epithelium | 0.12 | 0.87 | intron | ZSWIM5 | U |
| 41 | chr5 | 135370936 | 135371140 | Oral, Larynx and Esophageal epithelium | 0.11 | 0.86 | intron | TGFBI | U |
| 42 | chr4 | 113338667 | 113339055 | Oral, Larynx and Esophageal epithelium | 0.15 | 0.9 | intron | ALPK1 | U |
| 43 | chr16 | 18938831 | 18938862 | Oral, Larynx and Esophageal epithelium | 0.07 | 0.81 | Intergenic | SMG1 | U |
| 44 | chr11 | 16799587 | 16799678 | Oral, Larynx and Esophageal epithelium | 0.15 | 0.89 | Intergenic | C11orf58 | U |
| 45 | chr16 | 8850737 | 8850920 | Oral, Larynx and Esophageal epithelium | 0.12 | 0.86 | intron | ABAT | U |
| 46 | chr4 | 13587143 | 13587548 | Oral, Larynx and Esophageal epithelium | 0.14 | 0.88 | intron | BOD1L1 | U |
| 47 | chr5 | 18993241 | 18993458 | Oral, Larynx and Esophageal epithelium | 0.08 | 0.81 | Intergenic | CDH18 | U |
| 48 | chr5 | 167431793 | 167432185 | Oral, Larynx and Esophageal epithelium | 0.06 | 0.79 | intron | TENM2 | U |
| 49 | chr5 | 171042175 | 171042260 | Oral, Larynx and Esophageal epithelium | 0.11 | 0.83 | Intergenic | FGF18 | U |
| 50 | chr4 | 543478651 | 54348009 | Oral, Larynx and Esophageal epithelium | 0.2 | 0.92 | exon | LNX1 | U |
| 51 | chr12 | 116825665 | 116825821 | Oral, Larynx and Esophageal epithelium | 0.15 | 0.87 | Intergenic | MIR4472-2 | U |
| 52 | chr1 | 241419066 | 241419258 | Oral, Larynx and Esophageal epithelium | 0.16 | 0.88 | intron | RGS7 | U |
| 53 | chr7 | 105391774 | 105392015 | Oral, Larynx and Esophageal epithelium | 0.16 | 0.88 | intron | ATXN7L1 | U |
| 54 | chr19 | 39766779 | 39767161 | Oral, Larynx and Esophageal epithelium | 0.14 | 0.86 | Intergenic | IFNL2 | U |
| 55 | chr6 | 158619634 | 158619734 | Oral, Larynx and Esophageal epithelium | 0.19 | 0.9 | exon | GTF2H5 | U |
| 56 | chr6 | 83781122 | 83781223 | Oral, Larynx and Esophageal epithelium | 0.18 | 0.89 | intron | DOPEY1 | U |
| 57 | chr6 | 783511 | 783625 | Oral, Larynx and Esophageal epithelium | 0.15 | 0.86 | Intergenic | EXOC2 | U |
| 58 | chr8 | 134687594 | 134687716 | Oral, Larynx and Esophageal epithelium | 0.2 | 0.91 | Intergenic | ST3GAL1 | U |
| 59 | chr17 | 49227132 | 49227351 | Oral, Larynx and Esophageal epithelium | 0.18 | 0.89 | Intergenic | NME1-NME2 | U |
| 60 | chr11 | 60902945 | 60903186 | Oral, Larynx and Esophageal epithelium | 0.18 | 0.89 | intron | VPS37C | U |
| 61 | chr1 | 113740846 | 113741106 | Oral, Larynx and Esophageal epithelium | 0.12 | 0.83 | exon | LOC643441 | U |
| 62 | chr2 | 99649323 | 99649435 | Oral, Larynx and Esophageal epithelium | 0.19 | 0.88 | intron | TSGA10 | U |
| 63 | chr16 | 2998130 | 2998389 | Oral, Larynx and Esophageal epithelium | 0.15 | 0.84 | intron | FLYWCH1 | U |
| 64 | chr1 | 9426821 | 9427135 | Oral, Larynx and Esophageal epithelium | 0.21 | 0.9 | intron | SPSB1 | U |
| 65 | chr14 | 86143084 | 86143161 | Oral, Larynx and Esophageal epithelium | 0.21 | 0.89 | Intergenic | FLRT2 | U |
| 66 | chr16 | 1742392 | 1742538 | Oral, Larynx and Esophageal epithelium | 0.22 | 0.9 | intron | HN1L | U |
| 67 | chr3 | 58455461 | 58455629 | Oral, Larynx and Esophageal epithelium | 0.18 | 0.86 | Intergenic | KCTD6 | U |
| 68 | chr7 | 120066865 | 120066978 | Oral, Larynx and Esophageal epithelium | 0.25 | 0.92 | intron | KCND2 | U |
| 69 | chrX | 37096601 | 3709819 | Oral, Larynx and Esophageal epithelium | 0.2 | 0.87 | Intergenic | LOC389906 | U |
| 70 | chr16 | 18045493 | 1804755 | Oral, Larynx and Esophageal epithelium | 0.22 | 0.89 | intron | MAPK8IP3 | U |
| 71 | chr19 | 19081147 | 19081494 | Oral, Larynx and Esophageal epithelium | 0.19 | 0.86 | Intergenic | HOMER3 | U |
| 72 | chr5 | 159717995 | 159718380 | Oral, Larynx and Esophageal epithelium | 0.27 | 0.94 | intron | CCNJL | U |
| 73 | chr10 | 15414241 | 1541859 | Oral, Larynx and Esophageal epithelium | 0.15 | 0.82 | intron | ADARB2 | U |
| 74 | chrX | 36011273 | 3601616 | Oral, Larynx and Esophageal epithelium | 0.19 | 0.86 | intron | PRKX | U |
| 75 | chr17 | 378979983 | 37898156 | Oral, Larynx and Esophageal epithelium | 0.19 | 0.85 | intron | GRB7 | U |
| 76 | chr2 | 72255311 | 72255585 | Oral, Larynx and Esophageal epithelium | 0.25 | 0.91 | Intergenic | CYP26B1 | U |
| 77 | chr1 | 112090880 | 112091243 | Oral, Larynx and Esophageal epithelium | 0.22 | 0.88 | intron | ADORA3 | U |
| 78 | chr16 | 23548205 | 23548633 | Oral, Larynx and Esophageal epithelium | 0.25 | 0.91 | intron | EARS2 | U |
| 79 | chr19 | 11630205 | 11630292 | Oral, Larynx and Esophageal epithelium | 0.28 | 0.93 | intron | ECSIT | U |
| 80 | chr6 | 134717812 | 134718235 | Oral, Larynx and Esophageal epithelium | 0.28 | 0.93 | Intergenic | LOC154092 | U |
| 81 | chr17 | 36874349 | 36874501 | Oral, Larynx and Esophageal epithelium | 0.27 | 0.91 | exon, intron | MLLT6, MLLT6 | U |
| 82 | chr21 | 188761433 | 18876378 | Oral, Larynx and Esophageal epithelium | 0.26 | 0.89 | Intergenic | CXADR | U |
| 83 | chr10 | 73629716 | 73630010 | Oral, Larynx and Esophageal epithelium | 0.26 | 0.89 | Intergenic | PSAP | U |
| 84 | chr7 | 101651783 | 101652111 | Oral, Larynx and Esophageal epithelium | 0.23 | 0.85 | intron | CUX1 | U |
| 85 | chr2 | 1604579613 | 160458125 | Oral, Larynx and Esophageal epithelium | 0.32 | 0.93 | intron | BAZ2B | U |
| 86 | chr10 | 73630048 | 73630380 | Oral, Larynx and Esophageal epithelium | 0.29 | 0.9 | Intergenic | PSAP | U |
| 87 | chr22 | 46980960 | 46981213 | Oral, Larynx and Esophageal epithelium | 0.28 | 0.88 | Intergenic | GRAMD4 | U |
| 88 | chr19 | 6442589 | 6442809 | Oral, Larynx and Esophageal epithelium | 0.32 | 0.91 | intron | SLC25A23 | U |
| 89 | chr11 | 66881355 | 66881491 | Oral, Larynx and Esophageal epithelium | 0.31 | 0.87 | Intergenic | KDM2A | U |
| 90 | chr4 | 109465282 | 109465506 | Oral, Larynx and Esophageal epithelium | 0.34 | 0.88 | intron | RPL34-AS1 | U |
| 91 | chr20 | 51105019 | 51105183 | Oral, Larynx and Esophageal epithelium | 0.24 | 0.88 | Intergenic | ZFP64 | U |
| 92 | chr9 | 139546439 | 139546629 | Oral, Larynx and Esophageal epithelium | 0.03 | 0.91 | Intergenic | EGFL7 | U |
| 93 | chr7 | 1016525691 | 101652819 | Oral, Larynx and Esophageal epithelium | 0.06 | 0.92 | intron | CUX1 | U |
| 94 | chr22 | 47436553 | 47436863 | Oral, Larynx and Esophageal epithelium | 0.08 | 0.93 | intron | TBC1D22A | U |
| 95 | chr9 | 107109138 | 107109514 | Oral, Larynx and Esophageal epithelium | 0.06 | 0.9 | Intergenic | OR13F1 | U |
| 96 | chr20 | 44416524 | 44416740 | Oral, Larynx and Esophageal epithelium | 0.06 | 0.89 | exon, intron | WFDC3 | U |
| 97 | chr22 | 50954593 | 50954993 | Oral, Larynx and Esophageal epithelium | 0.09 | 0.89 | intron | NCAPH2 | U |
| 98 | chr2 | 65251128 | 65251226 | Oral, Larynx and Esophageal epithelium | 0.14 | 0.93 | TTS | SLC1A4 | U |
| 99 | chr2 | 121550957 | 121551277 | Oral, Larynx and Esophageal epithelium | 0.09 | 0.86 | Intergenic | GLI2 | U |
| 100 | chr22 | 336564653 | 33656602 | Oral, Larynx and Esophageal epithelium | 0.07 | 0.79 | Intergenic | MIR4764 | U |
| 101 | chr16 | 215692071 | 21569364 | Oral, Larynx and Esophageal epithelium | 0.23 | 0.9 | Intergenic | SLC7ASP2 | U |
| 102 | chr15 | 31289788 | 31290120 | Oral, Larynx and Esophageal epithelium | 0.1 | 0.94 | Intergenic | MTMR10 | U |
| 103 | chr2 | 2176744223 | 217674498 | Oral, Larynx and Esophageal epithelium | 0.09 | 0.92 | Intergenic | TNP1 | U |
| 104 | chr19 | 19214027 | 19214234 | Oral, Larynx and Esophageal epithelium | 0.06 | 0.89 | intron | SLC25A42 | U |
| 105 | chr9 | 88048026 | 88048312 | Oral, Larynx and Esophageal epithelium | 0.03 | 0.86 | Intergenic | AGTPBP1 | U |
| 106 | chr16 | 11342023 | 11342315 | Oral, Larynx and Esophageal epithelium | 0.03 | 0.86 | Intergenic | SOCS1 | U |
| 107 | chr22 | 24976072 | 24976253 | Oral, Larynx and Esophageal epithelium | 0.03 | 0.85 | Intergenic | GGT1 | U |
| 108 | chr7 | 5692480 | 5692793 | Oral, Larynx and Esophageal epithelium | 0.07 | 0.89 | intron | RNF216 | U |
| 109 | chr6 | 17575539 | 17575726 | Oral, Larynx and Esophageal epithelium | 0.07 | 0.88 | Intergenic | FAM8A1 | U |
| 110 | chr14 | 74849506 | 74849735 | Oral, Larynx and Esophageal epithelium | 0.09 | 0.88 | Intergenic | VRTN | U |
| 111 | chr16 | 39984213 | 3998887 | Oral, Larynx and Esophageal epithelium | 0.12 | 0.91 | Intergenic | CREBBP | U |
| 112 | chr13 | 113105830 | 113105972 | Oral, Larynx and Esophageal epithelium | 0.14 | 0.91 | Intergenic | SPACA7 | U |
| 113 | chr4 | 184554422 | 184554639 | Oral, Larynx and Esophageal epithelium | 0.16 | 0.93 | Intergenic | RWDD4 | U |
| 114 | chr4 | 184754711 | 184754932 | Oral, Larynx and Esophageal epithelium | 0.09 | 0.86 | Intergenic | STOX2 | U |
| 115 | chr2 | 190369207 | 190369538 | Oral, Larynx and Esophageal epithelium | 0.1 | 0.86 | Intergenic | WDR75 | U |
| 116 | chr20 | 35858645 | 35859093 | Oral, Larynx and Esophageal epithelium | 0.14 | 0.9 | intron | RPN2 | U |
| 117 | chr20 | 31649647 | 31649945 | Oral, Larynx and Esophageal epithelium | 0.1 | 0.82 | intron | BPIFB3 | U |
| 118 | chr14 | 74513552 | 74513683 | Oral, Larynx and Esophageal epithelium | 0.21 | 0.91 | intron | CCDC176 | U |
| 119 | chr20 | 33144418 | 33144780 | Oral, Larynx and Esophageal epithelium | 0.21 | 0.9 | intron | MAP1LC3A | U |
| 120 | chr2 | 47094735 | 47095109 | Oral, Larynx and Esophageal epithelium | 0.22 | 0.92 | Intergenic | LOC100134259 | U |
| 121 | chr4 | 1782780013 | 178278459 | Oral, Larynx and Esophageal epithelium | 0.21 | 0.9 | intron | NEIL3 | U |
| 122 | chr1 | 271832691 | 27183618 | Oral, Larynx and Esophageal epithelium | 0.19 | 0.86 | Intergenic | SFN | U |
| 123 | chr20 | 358576683 | 35858009 | Oral, Larynx and Esophageal epithelium | 0.24 | 0.9 | intron | RPN2 | U |
| 124 | chr6 | 16323477 | 16323635 | Oral, Larynx and Esophageal epithelium | 0.21 | 0.83 | intron | ATXN1 | U |
| 125 | chr22 | 18287102 | 18287302 | Oral, Larynx and Esophageal epithelium | 0.27 | 0.85 | intron | MICAL3 | U |
| 126 | chr4 | 86747319 | 86747660 | Oral, Larynx and Esophageal epithelium | 0.7 | 0.14 | intron | ARHGAP24 | M |
| 127 | chr5 | 139030310 | 139030623 | Oral, Larynx and Esophageal epithelium | 0.61 | 0.09 | intron | CXXC5 | M |
| 128 | chr16 | 1970516 | 1970659 | Oral, Larynx and Esophageal epithelium | 0.74 | 0.26 | Intergenic | HS3ST6 | M |
| 129 | chr8 | 94995343 | 94995468 | Oral, Larynx and Esophageal epithelium | 0.66 | 0.21 | Intergenic | PDP1 | M |
| 130 | chr1 | 226187637 | 226187770 | Oral, Larynx and Esophageal epithelium | 0.58 | 0.14 | promoter-TSS | SDE2 | M |
| 131 | chr19 | 52223282 | 52223427 | Oral, Larynx and Esophageal epithelium | 0.59 | 0.2 | intron | HAS1 | M |
| 132 | chr6 | 1330893223 | 133089520 | Oral, Larynx and Esophageal epithelium | 0.61 | 0.25 | Intergenic | VNN2 | M |
| 133 | chr21 | 43199238 | 43199497 | Oral, Larynx and Esophageal epithelium | 0.52 | 0.18 | Intergenic | RIPK4 | M |
| 134 | chr8 | 38299962 | 38300250 | Oral, Larynx and Esophageal epithelium | 0.57 | 0.13 | intron | FGFR1 | M |
| 135 | chr3 | 181421265 | 181421663 | Oral, Larynx and Esophageal epithelium | 0.79 | 0.12 | intron | SOX2-OT | M |
| 136 | chr20 | 55500006 | 55500149 | Oral, Larynx and Esophageal epithelium | 0.8 | 0.16 | Intergenic | TFAP2C | M |
| 137 | chr11 | 78673053 | 78673213 | Oral, Larynx and Esophageal epithelium | 0.81 | 0.19 | intron | TENM4 | M |
| 138 | chr17 | 80452508 | 80452915 | Oral, Larynx and Esophageal epithelium | 0.66 | 0.1 | Intergenic | FOXK2 | M |
| 139 | chr17 | 48049945 | 48049980 | Oral, Larynx and Esophageal epithelium | 0.73 | 0.19 | promoter-TSS | DLX4 | M |
| 140 | chr17 | 48050244 | 48050304 | Oral, Larynx and Esophageal epithelium | 0.72 | 0.2 | exon | DLX4 | M |
| 141 | chr9 | 96715977 | 96716101 | Oral, Larynx and Esophageal epithelium | 0.75 | 0.23 | intron | BARX1 | M |
| 142 | chr17 | 53341243 | 53341566 | Oral, Larynx and Esophageal epithelium | 0.65 | 0.13 | promoter-TSS, Interg [symbol] | HLF, HLF | M |
| 143 | chr16 | 3017432 | 3017558 | Oral, Larynx and Esophageal epithelium | 0.67 | 0.17 | intron, exon | KREMEN2, KREMEN2 | M |
| 144 | chr16 | 68676804 | 68676935 | Oral, Larynx and Esophageal epithelium | 0.62 | 0.13 | Intergenic | CDH3 | M |
| 145 | chr17 | 48050048 | 48050197 | Oral, Larynx and Esophageal epithelium | 0.63 | 0.15 | promoter-TSS | DLX4 | M |
| 146 | chr9 | 975762 | 975857 | Oral, Larynx and Esophageal epithelium | 0.72 | 0.25 | Intergenic | DMRT3 | M |
| 147 | chr13 | 95354825 | 95354866 | Oral, Larynx and Esophageal epithelium | 0.65 | 0.19 | Intergenic | SOX21 | M |
| 148 | chr21 | 28337857 | 28337912 | Oral, Larynx and Esophageal epithelium | 0.66 | 0.21 | exon | ADAMTS5 | M |
| 149 | chr9 | 96709470 | 96709768 | Oral, Larynx and Esophageal epithelium | 0.6 | 0.16 | Intergenic | BARX1 | M |
| 150 | chr10 | 102898947 | 102899157 | Oral, Larynx and Esophageal epithelium | 0.61 | 0.18 | Intergenic | TLX1 | M |
| 151 | chr6 | 126336351 | 126336655 | Gastric Epithelium | 0.05 | 0.97 | intron | TRMT11 | U |
| 152 | chr5 | 1396274463 | 139627765 | Gastric Epithelium | 0.03 | 0.95 | intron | PFDN1 | U |
| 153 | chr6 | 119154681 | 119155026 | Gastric Epithelium | 0.01 | 0.91 | intron | MCM9 | U |
| 154 | chr7 | 152166508 | 152166876 | Gastric Epithelium | 0.01 | 0.91 | Intergenic | LOC100128822 | U |
| 155 | chr2 | 232284718 | 232284780 | Gastric Epithelium | 0.06 | 0.95 | Intergenic | B3GNT7 | U |
| 156 | chr18 | 55321793 | 55321987 | Gastric Epithelium | 0.06 | 0.95 | intron | ATP8B1 | U |
| 157 | chr1 | 1508136163 | 150813721 | Gastric Epithelium | 0.05 | 0.93 | intron | ARNT | U |
| 158 | chr2 | 103095103 | 103095285 | Gastric Epithelium | 0.01 | 0.89 | intron | SLC9A4 | U |
| 159 | chr4 | 25304443 | 25304717 | Gastric Epithelium | 0.01 | 0.89 | Intergenic | ZCCHC4 | U |
| 160 | chr21 | 35738641 | 35739006 | Gastric Epithelium | 0.02 | 0.9 | intron | KCNE2 | U |
| 161 | chr3 | 30693303 | 30693494 | Gastric Epithelium | 0.06 | 0.93 | intron | TGFBR2 | U |
| 162 | chr21 | 35739053 | 35739403 | Gastric Epithelium | 0.04 | 0.91 | intron | KCNE2 | U |
| 163 | chr8 | 6595371 | 6595859 | Gastric Epithelium | 0.08 | 0.95 | intron | AGPAT5 | U |
| 164 | chr17 | 554292 | 554701 | Gastric Epithelium | 0.06 | 0.92 | intron | VPS53 | U |
| 165 | chr6 | 119150051 | 119150409 | Gastric Epithelium | 0.08 | 0.94 | exon | MCM9 | U |
| 166 | chr4 | 1364945 | 1365429 | Gastric Epithelium | 0.07 | 0.92 | intron | UVSSA | U |
| 167 | chr1 | 54805307 | 54805622 | Gastric Epithelium | 0.09 | 0.93 | intron | SSBP3 | U |
| 168 | chr13 | 22036153 | 22036385 | Gastric Epithelium | 0.1 | 0.93 | Intergenic | ZDHHC20 | U |
| 169 | chr1 | 32411721 | 32411803 | Gastric Epithelium | 0.17 | 0.96 | Intergenic | PTP4A2 | U |
| 170 | chr2 | 1128249101 | 112825170 | Gastric Epithelium | 0.16 | 0.94 | intron | TMEM87B | U |
| 171 | chr3 | 65168887 | 65168944 | Gastric Epithelium | 0.02 | 0.94 | Intergenic | MIR548A2 | U |
| 172 | chr12 | 77624848 | 77624911 | Gastric Epithelium | 0.01 | 0.93 | Intergenic | E2F7 | U |
| 173 | chr1 | 775703581 | 77570467 | Gastric Epithelium | 0.02 | 0.92 | intron | PIGK | U |
| 174 | chr1 | 41640685 | 41640931 | Gastric Epithelium | 0.01 | 0.91 | intron | SCMH1 | U |
| 175 | chr12 | 32651756 | 32651898 | Gastric Epithelium | 0.02 | 0.91 | Intergenic | FGD4 | U |
| 176 | chr3 | 137727159 | 137727360 | Gastric Epithelium | 0.02 | 0.91 | intron | CLDN18 | U |
| 177 | chr21 | 35736753 | 35736986 | Gastric Epithelium | 0.02 | 0.91 | intron | KCNE2 | U |
| 178 | chr21 | 35737943 | 35738215 | Gastric Epithelium | 0.01 | 0.9 | intron | KCNE2 | U |
| 179 | chr8 | 135939325 | 135939699 | Gastric Epithelium | 0.01 | 0.9 | Intergenic | MIR30D | U |
| 180 | chr20 | 58554277 | 58554745 | Gastric Epithelium | 0.02 | 0.91 | intron | CDH26 | U |
| 181 | chr20 | 51959785 | 51959856 | Gastric Epithelium | 0.02 | 0.9 | intron | TSHZ2 | U |
| 182 | chr11 | 77707568 | 77707699 | Gastric Epithelium | 0.03 | 0.91 | Intergenic | INTS4 | U |
| 183 | chr2 | 98376000 | 98376235 | Gastric Epithelium | 0.04 | 0.92 | exon | TMEM131 | U |
| 184 | chr13 | 74588468 | 74588895 | Gastric Epithelium | 0.04 | 0.92 | intron | KLF12 | U |
| 185 | chr5 | 127000250 | 127000320 | Gastric Epithelium | 0.01 | 0.88 | Intergenic | CTXN3 | U |
| 186 | chr4 | 184013404 | 184013610 | Gastric Epithelium | 0.07 | 0.94 | Intergenic | WWC2-AS2 | U |
| 187 | chr11 | 59303419 | 59303666 | Gastric Epithelium | 0.01 | 0.88 | Intergenic | OR4D9 | U |
| 188 | chr6 | 137024152 | 137024470 | Gastric Epithelium | 0.07 | 0.94 | intron | MAP3K5 | U |
| 189 | chr4 | 122607782 | 122608154 | Gastric Epithelium | 0.05 | 0.92 | intron | ANXA5 | U |
| 190 | chr11 | 47890596 | 47891033 | Gastric Epithelium | 0.04 | 0.91 | Intergenic | NUP160 | U |
| 191 | chr19 | 52683528 | 52684016 | Gastric Epithelium | 0.02 | 0.89 | Intergenic, Intergeni [symbol] | ZNF836, PPP2R1A | U |
| 192 | chr16 | 81295026 | 81295120 | Gastric Epithelium | 0.03 | 0.89 | intron | BCMO1 | U |
| 193 | chr1 | 38353963 | 38354186 | Gastric Epithelium | 0.06 | 0.92 | intron | INPP5B | U |
| 194 | chr12 | 80052815 | 80053053 | Gastric Epithelium | 0.09 | 0.95 | intron | PAWR | U |
| 195 | chr19 | 122692981 | 12269551 | Gastric Epithelium | 0.05 | 0.91 | Intergenic | ZNF625-ZNF20 | U |
| 196 | chr9 | 128467739 | 128468047 | Gastric Epithelium | 0.07 | 0.93 | intron | MAPKAP1 | U |
| 197 | chr1 | 235359359 | 235359698 | Gastric Epithelium | 0.07 | 0.93 | intron | ARID4B | U |
| 198 | chr6 | 130332560 | 130332959 | Gastric Epithelium | 0.07 | 0.93 | Intergenic | L3MBTL3 | U |
| 199 | chr1 | 246490876 | 246491294 | Gastric Epithelium | 0.07 | 0.93 | intron | SMYD3 | U |
| 200 | chr2 | 239252776 | 239253198 | Gastric Epithelium | 0.03 | 0.89 | intron | TRAF3IP1 | U |
| 201 | chr4 | 186492550 | 186492641 | Gastric Epithelium | 0.05 | 0.9 | Intergenic | PDLIM3 | U |
| 202 | chr2 | 52957376 | 52957515 | Gastric Epithelium | 0.09 | 0.94 | Intergenic | CHAC2 | U |
| 203 | chr7 | 98006036 | 98006197 | Gastric Epithelium | 0.02 | 0.87 | intron | BAIAP2L1 | U |
| 204 | chr13 | 34897662 | 34897830 | Gastric Epithelium | 0.03 | 0.88 | Intergenic | LINC00457 | U |
| 205 | chr3 | 194017474 | 194017652 | Gastric Epithelium | 0.07 | 0.92 | Intergenic | LOC100131551 | U |
| 206 | chr17 | 10704250 | 10704431 | Gastric Epithelium | 0.02 | 0.87 | intron | LINC00675 | U |
| 207 | chr10 | 490007 | 490215 | Gastric Epithelium | 0.07 | 0.92 | intron | DIP2C | U |
| 208 | chr2 | 128021537 | 128021766 | Gastric Epithelium | 0.05 | 0.9 | intron | ERCC3 | U |
| 209 | chr8 | 74278573 | 74278808 | Gastric Epithelium | 0.09 | 0.94 | Intergenic | STAU2-AS1 | U |
| 210 | chr4 | 25851481 | 25851741 | Gastric Epithelium | 0.04 | 0.89 | intron | SEL1L3 | U |
| 211 | chr16 | 30126072 | 30126338 | Gastric Epithelium | 0.04 | 0.89 | exon, intron | MAPK3, MAPK3 | U |
| 212 | chr4 | 25304099 | 25304397 | Gastric Epithelium | 0.05 | 0.91 | Intergenic | ZCCHC4 | U |
| 213 | chr6 | 41727035 | 41727454 | Gastric Epithelium | 0.02 | 0.87 | Intergenic | PGC | U |
| 214 | chr2 | 1583278583 | 158328298 | Gastric Epithelium | 0.04 | 0.89 | Intergenic | CYTIP | U |
| 215 | chr5 | 90287841 | 90288285 | Gastric Epithelium | 0.04 | 0.89 | intron | GPR98 | U |
| 216 | chr6 | 43630355 | 43630404 | Gastric Epithelium | 0.04 | 0.88 | intron | RSPH9 | U |
| 217 | chr3 | 8529090 | 8529169 | Gastric Epithelium | 0.06 | 0.9 | intron | LMCD1-AS1 | U |
| 218 | chr2 | 39017864 | 39018065 | Gastric Epithelium | 0.08 | 0.92 | Intergenic | GEMIN6 | U |
| 219 | chr15 | 42045333 | 42045534 | Gastric Epithelium | 0.06 | 0.9 | intron | MGA | U |
| 220 | chr2 | 11960587 | 11960862 | Gastric Epithelium | 0.07 | 0.91 | intron | LPIN1 | U |
| 221 | chr15 | 42149853 | 42150275 | Gastric Epithelium | 0.06 | 0.9 | exon, intron | SPT8N5, SPTBN5 | U |
| 222 | chr4 | 159964521 | 159964568 | Gastric Epithelium | 0.12 | 0.95 | Intergenic | C4orf45 | U |
| 223 | chr16 | 28984636 | 28984737 | Gastric Epithelium | 0.04 | 0.87 | Intergenic | SPNS1 | U |
| 224 | chr12 | 125410972 | 125411147 | Gastric Epithelium | 0.05 | 0.88 | Intergenic | MIR5188 | U |
| 225 | chr1 | 180988876 | 180989065 | Gastric Epithelium | 0.11 | 0.94 | intron | STX6 | U |
| 226 | chr14 | 93606446 | 93606669 | Gastric Epithelium | 0.02 | 0.85 | Intergenic | ITPK1 | U |
| 227 | chr19 | 38415210 | 38415464 | Gastric Epithelium | 0.07 | 0.9 | intron | SIPA1L3 | U |
| 228 | chr11 | 66120745 | 66121119 | Gastric Epithelium | 0.03 | 0.86 | Intergenic | B3GNT1 | U |
| 229 | chr3 | 29884926 | 29885392 | Gastric Epithelium | 0.06 | 0.89 | intron | RBMS3 | U |
| 230 | chr1 | 36354031 | 36354168 | Gastric Epithelium | 0.05 | 0.87 | exon | AGO1 | U |
| 231 | chr2 | 65413067 | 65413299 | Gastric Epithelium | 0.04 | 0.86 | Intergenic | ACTR2 | U |
| 232 | chr11 | 130515074 | 130515314 | Gastric Epithelium | 0.04 | 0.86 | Intergenic | C11orf44 | U |
| 233 | chr19 | 12416506 | 12416750 | Gastric Epithelium | 0.09 | 0.91 | Intergenic | ZNF44 | U |
| 234 | chr4 | 25304741 | 25305075 | Gastric Epithelium | 0.11 | 0.93 | Intergenic | ZCCHC4 | U |
| 235 | chr7 | 23380702 | 23381051 | Gastric Epithelium | 0.11 | 0.93 | intron | IGF2BP3 | U |
| 236 | chr11 | 16981380 | 16981822 | Gastric Epithelium | 0.02 | 0.84 | intron | PLEKHA7 | U |
| 237 | chr16 | 4253074 | 4253125 | Gastric Epithelium | 0.09 | 0.9 | exon, intron | SRL, SRL | U |
| 238 | chr1 | 160945988 | 160946061 | Gastric Epithelium | 0.11 | 0.92 | Intergenic | ITLN2 | U |
| 239 | chr4 | 85385908 | 85385981 | Gastric Epithelium | 0.08 | 0.89 | Intergenic | NKX6-1 | U |
| 240 | chr20 | 52682634 | 52682737 | Gastric Epithelium | 0.03 | 0.84 | intron | BCAS1 | U |
| 241 | chr17 | 380750741 | 38075221 | Gastric Epithelium | 0.1 | 0.91 | promoter-TSS | GSDMB | U |
| 242 | chr19 | 34933440 | 34933588 | Gastric Epithelium | 0.09 | 0.9 | intron | UBA2 | U |
| 243 | chr1 | 118189258 | 118189446 | Gastric Epithelium | 0.04 | 0.85 | Intergenic | FAM46C | U |
| 244 | chr3 | 1937569913 | 193757221 | Gastric Epithelium | 0.06 | 0.87 | Intergenic | LOC647323 | U |
| 245 | chr17 | 70409134 | 70409373 | Gastric Epithelium | 0.08 | 0.89 | intron | LINC00673 | U |
| 246 | chr12 | 122177926 | 122178189 | Gastric Epithelium | 0.1 | 0.91 | intron | TMEM120B | U |
| 247 | chr21 | 1713345 | 17133723 | Gastric Epithelium | 0.07 | 0.88 | intron | USP25 | U |
| 248 | chr1 | 617291013 | 61729405 | Gastric Epithelium | 0.12 | 0.93 | intron | NFIA | U |
| 249 | chr1 | 1586852 | 1587174 | Gastric Epithelium | 0.04 | 0.85 | intron | CDK11B | U |
| 250 | chr2 | 8427976 | 8428306 | Gastric Epithelium | 0.06 | 0.87 | intron | LINC00299 | U |
| 251 | chr1 | 67570737 | 67570839 | Gastric Epithelium | 0.09 | 0.89 | intron | C1orf141 | U |
| 252 | chr21 | 39899795 | 39899906 | Gastric Epithelium | 0.06 | 0.86 | intron | ERG | U |
| 253 | chr21 | 42504879 | 42505065 | Gastric Epithelium | 0.13 | 0.93 | Intergenic | LINC00323 | U |
| 254 | chr16 | 78883425 | 78883736 | Gastric Epithelium | 0.1 | 0.9 | intron | WWOK | U |
| 255 | chr16 | 75569829 | 75570175 | Gastric Epithelium | 0.07 | 0.87 | promoter-TSS, Interg [symbol] | CHST5, CHST5 | U |
| 256 | chr11 | 108055929 | 108056301 | Gastric Epithelium | 0.13 | 0.93 | intron | NPAT | U |
| 257 | chr3 | 33397874 | 33397945 | Gastric Epithelium | 0.14 | 0.93 | intron | FBXL2 | U |
| 258 | chr17 | 7904025 | 7904133 | Gastric Epithelium | 0.13 | 0.89 | Intergenic | GUCY2D | U |
| 259 | chr11 | 129742817 | 129742926 | Gastric Epithelium | 0.07 | 0.86 | exon | NFRKB | U |
| 260 | chr11 | 27617143 | 2761854 | Gastric Epithelium | 0.08 | 0.87 | intron | KONQ1 | U |
| 261 | chr18 | 47112271 | 47112453 | Gastric Epithelium | 0.08 | 0.87 | intron | LIPG | U |
| 262 | chr5 | 139642009 | 139642271 | Gastric Epithelium | 0.1 | 0.89 | intron | PFDN1 | U |
| 263 | chr1 | 110339894 | 110340165 | Gastric Epithelium | 0.1 | 0.89 | Intergenic | EPS8L3 | U |
| 264 | chr4 | 6958629 | 6958910 | Gastric Epithelium | 0.1 | 0.89 | intron | TBC1D14 | U |
| 265 | chr1 | 7738875 | 7739168 | Gastric Epithelium | 0.06 | 0.85 | intron | CAMTA1 | U |
| 266 | chr15 | 56737844 | 56738243 | Gastric Epithelium | 0.13 | 0.92 | TTS, intron | TEX9, MNS1 | U |
| 267 | chr20 | 62162500 | 62162591 | Gastric Epithelium | 0.03 | 0.81 | intron | PTK6 | U |
| 268 | chr6 | 41743174 | 41743288 | Gastric Epithelium | 0.13 | 0.91 | exon | FRS3 | U |
| 269 | chr6 | 47476304 | 47476438 | Gastric Epithelium | 0.07 | 0.85 | intron | CD2AP | U |
| 270 | chr6 | 345302313 | 34530402 | Gastric Epithelium | 0.12 | 0.9 | Intergenic | SPDEF | U |
| 271 | chr19 | 11995926 | 11996155 | Gastric Epithelium | 0.13 | 0.91 | Intergenic | ZNF69 | U |
| 272 | chr16 | 11359108 | 11359359 | Gastric Epithelium | 0.08 | 0.86 | Intergenic | TNP2 | U |
| 273 | chr3 | 619242011 | 61924583 | Gastric Epithelium | 0.14 | 0.92 | intron | PTPRG | U |
| 274 | chr15 | 38493152 | 38493607 | Gastric Epithelium | 0.11 | 0.89 | Intergenic | SPRED1 | U |
| 275 | chr19 | 1787588 | 1787677 | Gastric Epithelium | 0.04 | 0.81 | intron | ATP883 | U |
| 276 | chr15 | 99992318 | 99992424 | Gastric Epithelium | 0.14 | 0.91 | Intergenic | MEF2A | U |
| 277 | chr8 | 117703911 | 117704068 | Gastric Epithelium | 0.19 | 0.96 | intron | EIF3H | U |
| 278 | chr10 | 120931928 | 120932152 | Gastric Epithelium | 0.16 | 0.93 | intron | PRDX3 | U |
| 279 | chr19 | 105931543 | 10593276 | Gastric Epithelium | 0.15 | 0.91 | Intergenic | KEAP1 | U |
| 280 | chr3 | 194184924 | 194185088 | Gastric Epithelium | 0.17 | 0.93 | intron | ATP13A3 | U |
| 281 | chr8 | 39638417 | 39638589 | Gastric Epithelium | 0.08 | 0.84 | intron | ADAM2 | U |
| 282 | chr5 | 54813736 | 54813951 | Gastric Epithelium | 0.12 | 0.88 | intron | PPAP2A | U |
| 283 | chr3 | 196007281 | 196007517 | Gastric Epithelium | 0.13 | 0.86 | intron | PCYT1A | U |
| 284 | chr16 | 84599559 | 84599834 | Gastric Epithelium | 0.12 | 0.88 | exon | COTL1 | U |
| 285 | chr5 | 141560806 | 141561090 | Gastric Epithelium | 0.13 | 0.89 | Intergenic | NDFIP1 | U |
| 286 | chr22 | 49179647 | 49179852 | Gastric Epithelium | 0.05 | 0.8 | Intergenic | MIR4535 | U |
| 287 | chr5 | 149964103 | 149964516 | Gastric Epithelium | 0.13 | 0.85 | Intergenic | SYNPO | U |
| 288 | chr17 | 2710379 | 2710434 | Gastric Epithelium | 0.15 | 0.89 | intron | RAP1GAP2 | U |
| 289 | chr20 | 61005077 | 61005150 | Gastric Epithelium | 0.15 | 0.89 | Intergenic | RBBP8NL | U |
| 290 | chr1 | 163641818 | 163642065 | Gastric Epithelium | 0.18 | 0.92 | Intergenic | NUF2 | U |
| 291 | chr15 | 34654963 | 34655235 | Gastric Epithelium | 0.16 | 0.9 | exon, intron | LPCAT4, LPCAT4 | U |
| 292 | chr16 | 80737588 | 80737900 | Gastric Epithelium | 0.18 | 0.92 | intron | CDYL2 | U |
| 293 | chr1 | 232767194 | 232767531 | Gastric Epithelium | 0.12 | 0.86 | Intergenic | SIPA1L2 | U |
| 294 | chr17 | 46346042 | 46346172 | Gastric Epithelium | 0.09 | 0.82 | intron | SKAP1 | U |
| 295 | chr1 | 100443241 | 100443464 | Gastric Epithelium | 0.17 | 0.9 | intron | SLC35A3 | U |
| 296 | chr3 | 141945963 | 141946013 | Gastric Epithelium | 0.22 | 0.93 | Intergenic | GK5 | U |
| 297 | chr5 | 148991965 | 148992022 | Gastric Epithelium | 0.19 | 0.9 | intron | ARHGEF37 | U |
| 298 | chr3 | 47022705 | 47022847 | Gastric Epithelium | 0.11 | 0.82 | intron | NBEAL2 | U |
| 299 | chr5 | 7766437 | 7766579 | Gastric Epithelium | 0.17 | 0.88 | intron | ADCY2 | U |
| 300 | chr16 | 1445513 | 1445709 | Gastric Epithelium | 0.12 | 0.83 | intron | UNKL | U |
| 301 | chr5 | 35186960 | 35187169 | Gastric Epithelium | 0.18 | 0.89 | intron | PRLR | U |
| 302 | chr7 | 128693099 | 128693328 | Gastric Epithelium | 0.23 | 0.91 | intron | ENPO3 | U |
| 303 | chr6 | 131277389 | 131277448 | Gastric Epithelium | 0.24 | 0.94 | exon | EPB41L2 | U |
| 304 | chr1 | 80672272 | 80672333 | Gastric Epithelium | 0.21 | 0.91 | Intergenic | ELTD1 | U |
| 305 | chr12 | 122599860 | 122599964 | Gastric Epithelium | 0.24 | 0.94 | intron | MLXIP | U |
| 306 | chr2 | 28718991 | 28719286 | Gastric Epithelium | 0.18 | 0.88 | intron | PLB1 | U |
| 307 | chr6 | 39076099 | 39076529 | Gastric Epithelium | 0.14 | 0.84 | intron | SAYSD1 | U |
| 308 | chr16 | 11356838 | 11356943 | Gastric Epithelium | 0.18 | 0.87 | Intergenic | TNP2 | U |
| 309 | chr6 | 107647431 | 107647794 | Gastric Epithelium | 0.26 | 0.95 | intron | PDSS2 | U |
| 310 | chr17 | 1173324 | 1173401 | Gastric Epithelium | 0.18 | 0.86 | promoter-TSS | BALHA9 | U |
| 311 | chr11 | 107629594 | 107629752 | Gastric Epithelium | 0.14 | 0.82 | Intergenic | SLN | U |
| 312 | chr19 | 41677957 | 41678142 | Gastric Epithelium | 0.23 | 0.91 | Intergenic | CYP2S1 | U |
| 313 | chr6 | 148030997 | 148031401 | Gastric Epithelium | 0.22 | 0.9 | Intergenic | SAMD5 | U |
| 314 | chr7 | 102882090 | 102882162 | Gastric Epithelium | 0.23 | 0.9 | intron | DPY19L2P2 | U |
| 315 | chr7 | 953309 | 953429 | Gastric Epithelium | 0.12 | 0.79 | intron | ADAP1 | U |
| 316 | chr21 | 18801510 | 18801633 | Gastric Epithelium | 0.2 | 0.87 | Intergenic | C21orf37 | U |
| 317 | chr13 | 99827379 | 99827553 | Gastric Epithelium | 0.15 | 0.82 | Intergenic | UBAC2-AS1 | U |
| 318 | chr1 | 178476143 | 178476208 | Gastric Epithelium | 0.14 | 0.8 | Intergenic | FEX35 | U |
| 319 | chr7 | 97920858 | 97921050 | Gastric Epithelium | 0.25 | 0.91 | TTS, TTS | BAIAP2L1, BRI3 | U |
| 320 | chr12 | 112694142 | 112694609 | Gastric Epithelium | 0.26 | 0.92 | intron | HECTD4 | U |
| 321 | chr13 | 27822174 | 27822427 | Gastric Epithelium | 0.28 | 0.93 | Intergenic | RPL21 | U |
| 322 | chr1 | 7739328 | 7739594 | Gastric Epithelium | 0.16 | 0.81 | intron | CAMTA1 | U |
| 323 | chr10 | 1343073443 | 134307462 | Gastric Epithelium | 0.25 | 0.89 | Intergenic | NPP5A | U |
| 324 | chr8 | 85803401 | 8580388 | Gastric Epithelium | 0.16 | 0.79 | Intergenic | CLDN23 | U |
| 325 | chr3 | 13108236 | 13108359 | Gastric Epithelium | 0.21 | 0.84 | intron | QSEC1 | U |
| 326 | chr11 | 4197181 | 4197252 | Gastric Epithelium | 0.2 | 0.81 | Intergenic | LOC100506082 | U |
| 327 | chr2 | 441924891 | 44192747 | Gastric Epithelium | 0.3 | 0.89 | intron | LRPPRC | U |
| 328 | chr18 | 19694915 | 19695256 | Gastric Epithelium | 0.26 | 0.84 | Intergenic | GATA6 | U |
| 329 | chr3 | 182444733 | 182445118 | Gastric Epithelium | 0.27 | 0.83 | Intergenic | ATP11B | U |
| 330 | chr12 | 125359949 | 125360091 | Gastric Epithelium | 0.32 | 0.86 | Intergenic | SCARB1 | U |
| 331 | chr13 | 28625182 | 28625467 | Gastric Epithelium | 0.03 | 0.92 | intron | FLT3 | U |
| 332 | chr4 | 38397982 | 38398298 | Gastric Epithelium | 0.07 | 0.88 | Intergenic | KLF3 | U |
| 333 | chr7 | 1557915571 | 155791614 | Gastric Epithelium | 0.09 | 0.83 | Intergenic | SHH | U |
| 334 | chr3 | 127330153 | 12733108 | Gastric Epithelium | 0.18 | 0.91 | Intergenic | RAF1 | U |
| 335 | chr11 | 10847471 | 1084858 | Gastric Epithelium | 0.2 | 0.83 | exon | MUC2 | U |
| 336 | chr3 | 137717665 | 137717900 | Gastric Epithelium | 0.01 | 0.92 | exon | CLDN18 | U |
| 337 | chr9 | 71673940 | 71674378 | Gastric Epithelium | 0.04 | 0.95 | intron | FXN | U |
| 338 | chr2 | 103095409 | 103095901 | Gastric Epithelium | 0.02 | 0.91 | exon | SLC9A4 | U |
| 339 | chr15 | 59988949 | 59989041 | Gastric Epithelium | 0.07 | 0.91 | Intergenic | BNIP2 | U |
| 340 | chr14 | 50604935 | 50605125 | Gastric Epithelium | 0.13 | 0.96 | intron | SOS2 | U |
| 341 | chr13 | 28837971 | 28838117 | Gastric Epithelium | 0.03 | 0.96 | intron | PAN3 | U |
| 342 | chr22 | 41891370 | 41891750 | Gastric Epithelium | 0.02 | 0.92 | intron | ACO2 | U |
| 343 | chr11 | 57550804 | 57550988 | Gastric Epithelium | 0.03 | 0.89 | intron | CTNND1 | U |
| 344 | chr2 | 2415449013 | 241545393 | Gastric Epithelium | 0.01 | 0.87 | intron | GPR35 | U |
| 345 | chr1 | 1094990 | 1095143 | Gastric Epithelium | 0.03 | 0.88 | Intergenic | MIR200B | U |
| 346 | chr22 | 31616670 | 31616843 | Gastric Epithelium | 0.06 | 0.91 | intron | LIMK2 | U |
| 347 | chr17 | 57925572 | 57925756 | Gastric Epithelium | 0.02 | 0.87 | Intergenic | MIR21 | U |
| 348 | chr11 | 129742385 | 129742665 | Gastric Epithelium | 0.03 | 0.88 | intron | NFRKB | U |
| 349 | chr20 | 6760589 | 6760945 | Gastric Epithelium | 0.04 | 0.89 | exon, TTS | BMP2, BMP2 | U |
| 350 | chr1 | 1062975 | 1063187 | Gastric Epithelium | 0.02 | 0.86 | Intergenic | LOC254099 | U |
| 351 | chr14 | 69643149 | 69643543 | Gastric Epithelium | 0.07 | 0.91 | Intergenic | EXD2 | U |
| 352 | chr7 | 134116404 | 134116558 | Gastric Epithelium | 0.07 | 0.9 | Intergenic | AKR1B1 | U |
| 353 | chr9 | 97369629 | 97369825 | Gastric Epithelium | 0.08 | 0.91 | intron | FBP1 | U |
| 354 | chr6 | 41709978 | 41710195 | Gastric Epithelium | 0.08 | 0.91 | exon | PGC | U |
| 355 | chr6 | 157431621 | 157432058 | Gastric Epithelium | 0.09 | 0.92 | intron | ARID1B | U |
| 356 | chr14 | 56508705 | 56509187 | Gastric Epithelium | 0.04 | 0.87 | Intergenic | PELI2 | U |
| 357 | chr1 | 231104748 | 231104865 | Gastric Epithelium | 0.09 | 0.9 | intron | TTC13 | U |
| 358 | chr6 | 24752424 | 24752713 | Gastric Epithelium | 0.1 | 0.9 | Intergenic | GMNN | U |
| 359 | chr22 | 32805994 | 32806319 | Gastric Epithelium | 0.09 | 0.89 | intron | C22orf28 | U |
| 360 | chr20 | 62662346 | 62662467 | Gastric Epithelium | 0.08 | 0.87 | intron | PRPF6 | U |
| 361 | chr9 | 114579556 | 114579749 | Gastric Epithelium | 0.14 | 0.93 | Intergenic | C9orf84 | U |
| 362 | chr4 | 543579543 | 54358206 | Gastric Epithelium | 0.12 | 0.91 | intron | NX1 | U |
| 363 | chr9 | 114651409 | 114651885 | Gastric Epithelium | 0.14 | 0.92 | Intergenic | UGCG | U |
| 364 | chr1 | 1062668 | 1062808 | Gastric Epithelium | 0.12 | 0.88 | Intergenic | LOC254099 | U |
| 365 | chr9 | 94937344 | 94937635 | Gastric Epithelium | 0.18 | 0.92 | Intergenic | LINC00475 | U |
| 366 | chr2 | 65413988 | 65414139 | Gastric Epithelium | 0.17 | 0.89 | Intergenic | ACTR2 | U |
| 367 | chr20 | 33628551 | 33628745 | Gastric Epithelium | 0.15 | 0.87 | intron | TRPC4AP | U |
| 368 | chr6 | 2244189 | 2244471 | Gastric Epithelium | 0.21 | 0.92 | intron | GMDS | U |
| 369 | chr20 | 30843838 | 30844028 | Gastric Epithelium | 0.17 | 0.87 | Intergenic | KIF3B | U |
| 370 | chr16 | 88683395 | 88683851 | Gastric Epithelium | 0.18 | 0.88 | intron | ZC3H18 | U |
| 371 | chr19 | 41673732 | 41673927 | Gastric Epithelium | 0.15 | 0.82 | Intergenic | CYP2S1 | U |
| 372 | chr9 | 986570661 | 98657262 | Gastric Epithelium | 0.29 | 0.96 | intron | ERCC6L2 | U |
| 373 | chr20 | 33997810 | 33998065 | Gastric Epithelium | 0.16 | 0.83 | intron | UQCC | U |
| 374 | chr5 | 175832583 | 175832719 | Gastric Epithelium | 0.17 | 0.83 | intron | CLTB | U |
| 375 | chr9 | 100820273 | 100820676 | Gastric Epithelium | 0.25 | 0.89 | intron | NANS | U |
| 376 | chr22 | 50885408 | 50885506 | Gastric Epithelium | 0.27 | 0.86 | exor | SBF1 | U |
| 377 | chr17 | 38844775 | 38844988 | Gastric Epithelium | 0.29 | 0.87 | Intergenic | KRT24 | U |
| 378 | chr9 | 97385813 | 97386052 | Gastric Epithelium | 0.26 | 0.82 | intron | FBP1 | U |
| 379 | chr5 | 138729351 | 138729653 | Gastric Epithelium | 0.88 | 0.16 | exon | PROB1 | M |
| 380 | chr15 | 82432262 | 82432468 | Gastric Epithelium | 0.83 | 0.12 | intron | EFTUD1 | M |
| 381 | chr10 | 32665666 | 32665921 | Gastric Epithelium | 0.77 | 0.11 | intron | EPC1 | M |
| 382 | chrX | 105421549 | 105421612 | Gastric Epithelium | 0.79 | 0.23 | intron | MUM1L1 | M |
| 383 | chr3 | 181417368 | 181417614 | Gastric Epithelium | 0.71 | 0.15 | exon | SOX2-OT | M |
| 384 | chr19 | 33784296 | 33784610 | Gastric Epithelium | 0.67 | 0.12 | Intergenic | CEBPA | M |
| 385 | chr19 | 6057263 | 6057449 | Gastric Epithelium | 0.68 | 0.14 | intron | RFX2 | M |
| 386 | chr7 | 158261071 | 158261181 | Gastric Epithelium | 0.65 | 0.13 | intron | PTPRN2 | M |
| 387 | chr1 | 207494170 | 207494238 | Gastric Epithelium | 0.73 | 0.23 | promoter-TSS | CD55 | M |
| 388 | chr8 | 38644209 | 38644336 | Gastric Epithelium | 0.59 | 0.09 | promoter-TSS | TACC1 | M |
| 389 | chr8 | 144650650 | 144650805 | Gastric Epithelium | 0.69 | 0.2 | exon | MROH6 | M |
| 390 | chr12 | 103695910 | 103696073 | Gastric Epithelium | 0.64 | 0.15 | exon | C12orf42 | M |
| 391 | chr1 | 228402009 | 228402035 | Gastric Epithelium | 0.73 | 0.29 | promoter-TSS | C1orf145 | M |
| 392 | chr13 | 70682525 | 70682653 | Gastric Epithelium | 0.68 | 0.24 | promoter-TSS | KLHL1 | M |
| 393 | chr6 | 40722284 | 40722343 | Gastric Epithelium | 0.67 | 0.24 | Intergenic | LRFN2 | M |
| 394 | chr11 | 65184712 | 65184910 | Gastric Epithelium | 0.63 | 0.22 | Intergenic | NEAT1 | M |
| 395 | chrX | 108843476 | 108843505 | Gastric Epithelium | 0.62 | 0.24 | Intergenic | KCNE1L | M |
| 396 | chr17 | 37400316 | 37400485 | Gastric Epithelium | 0.55 | 0.18 | Intergenic | STAC2 | M |
| 397 | chr1 | 47999397 | 47999460 | Gastric Epithelium | 0.57 | 0.21 | Intergenic | FOXD2 | M |
| 398 | chr19 | 9832712 | 9832818 | Gastric Epithelium | 0.58 | 0.22 | Intergenic | ZNF812 | M |
| 399 | chr3 | 13908233 | 13908303 | Gastric Epithelium | 0.58 | 0.27 | intron | WNT7A | M |
| 400 | chr19 | 49112138 | 49112279 | Gastric Epithelium | 0.59 | 0.28 | intron | FAM83E | M |
| 401 | chr17 | 7967499 | 7967918 | Gastric Epithelium | 0.58 | 0.27 | Intergenic | ALOX128 | M |
| 402 | chr8 | 86351007 | 86351135 | Gastric Epithelium | 0.84 | 0.1 | promoter-TSS | CA3 | M |
| 403 | chr6 | 1624957 | 1625250 | Gastric Epithelium | 0.92 | 0.09 | intron | GMDS | M |
| 404 | chr13 | 95360462 | 95360865 | Gastric Epithelium | 0.89 | 0.1 | Intergenic | SOX21 | M |
| 405 | chr5 | 50674046 | 50674313 | Gastric Epithelium | 0.92 | 0.15 | exon | LOC642366 | M |
| 406 | chr6 | 1625273 | 1625609 | Gastric Epithelium | 0.83 | 0.06 | intron | GMDS | M |
| 407 | chr8 | 86350667 | 86350970 | Gastric Epithelium | 0.82 | 0.06 | promoter-TSS | CA3 | M |
| 408 | chr5 | 138729829 | 138730141 | Gastric Epithelium | 0.82 | 0.08 | exon | PROB1 | M |
| 409 | chr8 | 86350423 | 86350633 | Gastric Epithelium | 0.88 | 0.15 | promoter-TSS | CA3 | M |
| 410 | chr6 | 1625627 | 1625757 | Gastric Epithelium | 0.84 | 0.16 | intron | GMDS | M |
| 411 | chr14 | 21091267 | 21091423 | Gastric Epithelium | 0.85 | 0.2 | Intergenic | OR6S1 | M |
| 412 | chr5 | 50695414 | 50695610 | Gastric Epithelium | 0.74 | 0.09 | Intergenic | LOC642366 | M |
| 413 | chr15 | 96952287 | 96952441 | Gastric Epithelium | 0.77 | 0.15 | Intergenic | MIR1469 | M |
| 414 | chr7 | 155595558 | 155595590 | Gastric Epithelium | 0.7 | 0.09 | TTS | SHH | M |
| 415 | chr11 | 46317357 | 46317804 | Gastric Epithelium | 0.68 | 0.07 | intron | CREB3L1 | M |
| 416 | chr5 | 50262917 | 50263096 | Gastric Epithelium | 0.75 | 0.15 | Intergenic | PARP8 | M |
| 417 | chr2 | 233755749 | 233756039 | Gastric Epithelium | 0.76 | 0.16 | intron | NGEF | M |
| 418 | chr14 | 21091179 | 21091220 | Gastric Epithelium | 0.81 | 0.23 | Intergenic | OR6S1 | M |
| 419 | chr5 | 7395532 | 7395755 | Gastric Epithelium | 0.68 | 0.11 | promoter-TSS | ADCY2 | M |
| 420 | chr12 | 1639108 | 1639152 | Gastric Epithelium | 0.74 | 0.2 | Intergenic | LOC100292680 | M |
| 421 | chr7 | 154705815 | 154705890 | Gastric Epithelium | 0.65 | 0.12 | Intergenic | LOC100132707 | M |
| 422 | chr20 | 29550850 | 29550949 | Gastric Epithelium | 0.75 | 0.22 | Intergenic | FRG1B | M |
| 423 | chr3 | 128765012 | 128765057 | Gastric Epithelium | 0.71 | 0.22 | Intergenic | GP9 | M |
| 424 | chr19 | 1775351 | 1775487 | Gastric Epithelium | 0.65 | 0.17 | TTS | ONECUT3 | M |
| 425 | chr5 | 149461437 | 149461585 | Gastric Epithelium | 0.67 | 0.19 | intron | CSF1R | M |
| 426 | chr14 | 94226109 | 94226495 | Gastric Epithelium | 0.62 | 0.14 | intron | PRIMA1 | M |
| 427 | chr8 | 102038446 | 102038850 | Gastric Epithelium | 0.58 | 0.14 | Intergenic | FLI42969 | M |
| 428 | chr3 | 128764857 | 128764997 | Gastric Epithelium | 0.57 | 0.15 | Intergenic | GP9 | M |
| 429 | chr10 | 19338949 | 19339311 | Small Intestine Epithelium | 0.01 | 0.93 | Intergenic | ARL5B | U |
| 430 | chr10 | 126496740 | 126497034 | Small Intestine Epithelium | 0.05 | 0.95 | intron | FAM175B | U |
| 431 | chr11 | 65594327 | 65594630 | Small Intestine Epithelium | 0.02 | 0.91 | Intergenic | SNX32 | U |
| 432 | chr13 | 30110150 | 30110574 | Small Intestine Epithelium | 0.04 | 0.93 | intron | SLC7A1 | U |
| 433 | chr11 | 440653 | 440758 | Small Intestine Epithelium | 0.06 | 0.94 | intron | ANO9 | U |
| 434 | chr2 | 1365949071 | 136595048 | Small Intestine Epithelium | 0.06 | 0.94 | promoter-TSS | LCT | U |
| 435 | chr4 | 178322231 | 178322466 | Small Intestine Epithelium | 0.07 | 0.94 | Intergenic | AGA | U |
| 436 | chr11 | 272256 | 272628 | Small Intestine Epithelium | 0.02 | 0.88 | Intergenic | NLRP6 | U |
| 437 | chr1 | 227290003 | 227290462 | Small Intestine Epithelium | 0.07 | 0.92 | intron | CDC42BPA | U |
| 438 | chr11 | 440819 | 440894 | Small Intestine Epithelium | 0.05 | 0.9 | intron | ANO9 | U |
| 439 | chr11 | 283462 | 283608 | Small Intestine Epithelium | 0.04 | 0.89 | intron | NLRP6 | U |
| 440 | chr2 | 163772524 | 163772792 | Small Intestine Epithelium | 0.03 | 0.88 | Intergenic | KCNH7 | U |
| 441 | chr5 | 79658542 | 79658774 | Small Intestine Epithelium | 0.02 | 0.86 | Intergenic | CRSP8P | U |
| 442 | chr5 | 1368683 | 1368878 | Small Intestine Epithelium | 0.06 | 0.89 | Intergenic | CLPTM1L | U |
| 443 | chr6 | 42964029 | 42964188 | Small Intestine Epithelium | 0.14 | 0.93 | intron | PPP2R5D | U |
| 444 | chr2 | 39376566 | 39376760 | Small Intestine Epithelium | 0.15 | 0.94 | Intergenic | SOS1 | U |
| 445 | chr15 | 81668644 | 81669077 | Small Intestine Epithelium | 0.15 | 0.93 | Intergenic | TMC3 | U |
| 446 | chr16 | 2216470 | 2216843 | Small Intestine Epithelium | 0.17 | 0.94 | intron | TRAF7 | U |
| 447 | chr12 | 2506614 | 2506665 | Small Intestine Epithelium | 0.03 | 0.93 | intron | CACNA1C | U |
| 448 | chr20 | 60397537 | 60397571 | Small Intestine Epithelium | 0.02 | 0.91 | intron | CDH4 | U |
| 449 | chr2 | 174574144 | 174574409 | Small Intestine Epithelium | 0.03 | 0.92 | Intergenic | SP3 | U |
| 450 | chr15 | 76793291 | 76793366 | Small Intestine Epithelium | 0.07 | 0.95 | intron | SCAPER | U |
| 451 | chr1 | 200073859 | 200074011 | Small Intestine Epithelium | 0.03 | 0.91 | intron | NR5A2 | U |
| 452 | chr13 | 45339685 | 45339918 | Small Intestine Epithelium | 0.02 | 0.9 | Intergenic | LINC00330 | U |
| 453 | chr3 | 23576826 | 23577178 | Small Intestine Epithelium | 0.06 | 0.94 | intron | UBE2E2 | U |
| 454 | chr14 | 103002787 | 103002864 | Small Intestine Epithelium | 0.01 | 0.88 | Intergenic | MIR4309 | U |
| 455 | chr5 | 137933890 | 137933983 | Small Intestine Epithelium | 0.03 | 0.9 | Intergenic | HSPA9 | U |
| 456 | chr17 | 433425043 | 43342664 | Small Intestine Epithelium | 0.02 | 0.89 | intron | MAP3K14 | U |
| 457 | chr3 | 100331802 | 100331981 | Small Intestine Epithelium | 0.05 | 0.91 | intron | GPR128 | U |
| 458 | chr4 | 57133379 | 57133580 | Small Intestine Epithelium | 0.07 | 0.91 | intron | KIAA1211 | U |
| 459 | chr11 | 77304398 | 77304508 | Small Intestine Epithelium | 0.09 | 0.92 | intron | AQP11 | U |
| 460 | chr7 | 151585473 | 151585601 | Small Intestine Epithelium | 0.03 | 0.86 | Intergenic | PRKAG2-AS1 | U |
| 461 | chr16 | 46964009 | 46964202 | Small Intestine Epithelium | 0.08 | 0.91 | exon | GPT2 | U |
| 462 | chr14 | 90111701 | 90111939 | Small Intestine Epithelium | 0.12 | 0.95 | Intergenic | FOXN3-AS2 | U |
| 463 | chr5 | 77971393 | 77971651 | Small Intestine Epithelium | 0.08 | 0.91 | Intergenic | LHFPL2 | U |
| 464 | chr11 | 271883 | 272180 | Small Intestine Epithelium | 0.03 | 0.86 | Intergenic | NLRP6 | U |
| 465 | chr7 | 155922310 | 155922367 | Small Intestine Epithelium | 0.07 | 0.89 | Intergenic | LOC285889 | U |
| 466 | chr16 | 2221849 | 2222065 | Small Intestine Epithelium | 0.1 | 0.92 | intron | TRAF7 | U |
| 467 | chr1 | 148004493 | 148004723 | Small Intestine Epithelium | 0.05 | 0.87 | exon | NBPF14 | U |
| 468 | chr17 | 76733982 | 76734353 | Small Intestine Epithelium | 0.04 | 0.86 | intron | CYTH1 | U |
| 469 | chr4 | 82368581 | 82368971 | Small Intestine Epithelium | 0.09 | 0.91 | intron | RASGEF1B | U |
| 470 | chr11 | 3170718 | 3170822 | Small Intestine Epithelium | 0.08 | 0.89 | intron | OSBPL5 | U |
| 471 | chr19 | 46683676 | 46683995 | Small Intestine Epithelium | 0.07 | 0.88 | Intergenic | DKFZp434J0226 | U |
| 472 | chr12 | 124678953 | 124679007 | Small Intestine Epithelium | 0.12 | 0.92 | intron | ZNF664-FAM101A | U |
| 473 | chr5 | 175977376 | 175977478 | Small Intestine Epithelium | 0.03 | 0.83 | intron | CDHR2 | U |
| 474 | chr12 | 9695085 | 9695225 | Small Intestine Epithelium | 0.08 | 0.88 | Intergenic | KLRB1 | U |
| 475 | chr8 | 81019211 | 81019366 | Small Intestine Epithelium | 0.08 | 0.88 | intron | TPD52 | U |
| 476 | chr5 | 173969345 | 173969504 | Small Intestine Epithelium | 0.04 | 0.84 | Intergenic | MSX2 | U |
| 477 | chr16 | 2216894 | 2217169 | Small Intestine Epithelium | 0.02 | 0.82 | intron | TRAF7 | U |
| 478 | chr12 | 93223439 | 93223843 | Small Intestine Epithelium | 0.13 | 0.93 | intron | EEA1 | U |
| 479 | chr13 | 30454758 | 30455209 | Small Intestine Epithelium | 0.12 | 0.92 | intron | LINC00297 | U |
| 480 | chr19 | 39350308 | 39350342 | Small Intestine Epithelium | 0.04 | 0.83 | Intergenic | HNRNPL | U |
| 481 | chr5 | 133337095 | 133337179 | Small Intestine Epithelium | 0.12 | 0.91 | intron | VDAC1 | U |
| 482 | chr5 | 180417016 | 180417115 | Small Intestine Epithelium | 0.09 | 0.88 | intron | BTNL3 | U |
| 483 | chr7 | 44370390 | 44370498 | Small Intestine Epithelium | 0.11 | 0.9 | Intergenic | CAMK2B | U |
| 484 | chr17 | 13201687 | 13201865 | Small Intestine Epithelium | 0.09 | 0.88 | Intergenic | ELAC2 | U |
| 485 | chr1 | 78404057 | 78404256 | Small Intestine Epithelium | 0.14 | 0.93 | intron | NEXN | U |
| 486 | chr19 | 8097523 | 8097903 | Small Intestine Epithelium | 0.1 | 0.89 | Intergenic | CCL25 | U |
| 487 | chr8 | 18219427 | 18219813 | Small Intestine Epithelium | 0.09 | 0.88 | Intergenic | NAT2 | U |
| 488 | chr6 | 168188398 | 168188823 | Small Intestine Epithelium | 0.09 | 0.88 | intron | C6orf123 | U |
| 489 | chr3 | 196363243 | 196363440 | Small Intestine Epithelium | 0.04 | 0.82 | Intergenic | LRRC33 | U |
| 490 | chr1 | 7739607 | 7739847 | Small Intestine Epithelium | 0.13 | 0.91 | intron | CAMTA1 | U |
| 491 | chr19 | 4843090 | 4843346 | Small Intestine Epithelium | 0.14 | 0.92 | intron | PLIN3 | U |
| 492 | chr17 | 77959558 | 77959815 | Small Intestine Epithelium | 0.1 | 0.88 | intron | TBC1D16 | U |
| 493 | chr17 | 4945025 | 4945311 | Small Intestine Epithelium | 0.13 | 0.91 | Intergenic | SLC52A1 | U |
| 494 | chr12 | 115890506 | 115890829 | Small Intestine Epithelium | 0.11 | 0.89 | Intergenic | MIR620 | U |
| 495 | chr2 | 197765431 | 197765608 | Small Intestine Epithelium | 0.16 | 0.93 | intron | PGAP1 | U |
| 496 | chr4 | 24179596 | 24179855 | Small Intestine Epithelium | 0.15 | 0.92 | Intergenic | PPARGC1A | U |
| 497 | chr15 | 52390924 | 52391211 | Small Intestine Epithelium | 0.17 | 0.94 | Intergenic | BCL2L10 | U |
| 498 | chr16 | 50848512 | 50848836 | Small Intestine Epithelium | 0.05 | 0.82 | Intergenic | CYLD | U |
| 499 | chr1 | 199747283 | 199747370 | Small Intestine Epithelium | 0.08 | 0.84 | Intergenic | NR5A2 | U |
| 500 | chr9 | 135481768 | 135481879 | Small Intestine Epithelium | 0.13 | 0.86 | intron | DDX31 | U |
| 501 | chr19 | 17441650 | 17441933 | Small Intestine Epithelium | 0.13 | 0.89 | exon | ANO8 | U |
| 502 | chr6 | 42352982 | 42353335 | Small Intestine Epithelium | 0.12 | 0.88 | intron | TRERF1 | U |
| 503 | chr2 | 10163043 | 10163411 | Small Intestine Epithelium | 0.09 | 0.85 | Intergenic | KLF11 | U |
| 504 | chr2 | 39456237 | 39456704 | Small Intestine Epithelium | 0.08 | 0.84 | intron | CDKL4 | U |
| 505 | chr20 | 61281101 | 61281268 | Small Intestine Epithelium | 0.14 | 0.89 | intron | SLCO4A1 | U |
| 506 | chr16 | 58690638 | 58690915 | Small Intestine Epithelium | 0.12 | 0.87 | Intergenic | CNOT1 | U |
| 507 | chr1 | 270682 | 270970 | Small Intestine Epithelium | 0.13 | 0.85 | Intergenic | NLRP6 | U |
| 508 | chr1 | 45558202 | 45558523 | Small Intestine Epithelium | 0.15 | 0.9 | intron | ZSWIM5 | U |
| 509 | chr7 | 148160083 | 148160287 | Small Intestine Epithelium | 0.19 | 0.93 | Intergenic | C7orf33 | U |
| 510 | chr19 | 7505487 | 7505692 | Small Intestine Epithelium | 0.16 | 0.9 | intron | ARHGEF18 | U |
| 511 | chr11 | 271009 | 271488 | Small Intestine Epithelium | 0.12 | 0.86 | Intergenic | NLRP6 | U |
| 512 | chr14 | 100641770 | 100641938 | Small Intestine Epithelium | 0.14 | 0.87 | Intergenic | DEGS2 | U |
| 513 | chr19 | 17891999 | 17892167 | Small Intestine Epithelium | 0.07 | 0.79 | exon, intron | FCHO1, FCHO1 | U |
| 514 | chr11 | 286423 | 286784 | Small Intestine Epithelium | 0.21 | 0.93 | Intergenic | ATHL1 | U |
| 515 | chr8 | 126415624 | 126415993 | Small Intestine Epithelium | 0.17 | 0.89 | Intergenic | TRIB1 | U |
| 516 | chr6 | 433900051 | 43390319 | Small Intestine Epithelium | 0.16 | 0.87 | Intergenic | ABCC10 | U |
| 517 | chr1 | 15547962 | 15548286 | Small Intestine Epithelium | 0.17 | 0.88 | Intergenic | FHAD1 | U |
| 518 | chr15 | 34636879 | 34637206 | Small Intestine Epithelium | 0.21 | 0.91 | Intergenic, promoter | NOP10, C15orf55 | U |
| 519 | chr5 | 40417359 | 40417526 | Small Intestine Epithelium | 0.17 | 0.87 | Intergenic | PTGER4 | U |
| 520 | chr9 | 123528706 | 123528951 | Small Intestine Epithelium | 0.24 | 0.94 | intron | FBXW2 | U |
| 521 | chr19 | 4907943 | 4908266 | Small Intestine Epithelium | 0.24 | 0.93 | Intergenic | UHRF1 | U |
| 522 | chr17 | 27167910 | 27168299 | Small Intestine Epithelium | 0.26 | 0.95 | intron | FAM222B | U |
| 523 | chr6 | 105563790 | 105564168 | Small Intestine Epithelium | 0.15 | 0.83 | intron | BVES | U |
| 524 | chr10 | 116682964 | 116683134 | Small Intestine Epithelium | 0.28 | 0.95 | Intergenic | TRUB1 | U |
| 525 | chr7 | 97834644 | 97835048 | Small Intestine Epithelium | 0.28 | 0.95 | exon | LMTK2 | U |
| 526 | chr17 | 30135449 | 30135540 | Small Intestine Epithelium | 0.32 | 0.95 | Intergenic | COPRS | U |
| 527 | chr12 | 6654337 | 6654781 | Small Intestine Epithelium | 0.31 | 0.92 | intron | IFFO1 | U |
| 528 | chr7 | 55162627 | 55162868 | Small Intestine Epithelium | 0.05 | 0.87 | intron | EGFR | U |
| 529 | chr1 | 3210411 | 3210462 | Small Intestine Epithelium | 0.09 | 0.85 | intron | PRDM16 | U |
| 530 | chr7 | 2884997 | 2885040 | Small Intestine Epithelium | 0.04 | 0.95 | Intergenic | GNA12 | U |
| 531 | chr9 | 79238335 | 79238460 | Small Intestine Epithelium | 0.06 | 0.92 | intron | PRUNE2 | U |
| 532 | chr7 | 100877583 | 100877927 | Small Intestine Epithelium | 0.06 | 0.92 | intron | CLDN15 | U |
| 533 | chr17 | 9123039 | 9123167 | Small Intestine Epithelium | 0.03 | 0.88 | intron | NTN1 | U |
| 534 | chr20 | 40727554 | 40727871 | Small Intestine Epithelium | 0.06 | 0.87 | intron | PTPRT | U |
| 535 | chr22 | 35759383 | 35759642 | Small Intestine Epithelium | 0.13 | 0.94 | Intergenic | HMOX1 | U |
| 536 | chr6 | 4263418 | 4263700 | Small Intestine Epithelium | 0.13 | 0.92 | Intergenic | ECI2 | U |
| 537 | chr6 | 34208627 | 34208812 | Small Intestine Epithelium | 0.05 | 0.94 | intron | HMGA1 | U |
| 538 | chr7 | 100883380 | 100883692 | Small Intestine Epithelium | 0.04 | 0.93 | intron | FIS1 | U |
| 539 | chr6 | 11753073 | 11753216 | Small Intestine Epithelium | 0.02 | 0.9 | intron | ADTRP | U |
| 540 | chr2 | 235894334 | 23589482 | Small Intestine Epithelium | 0.07 | 0.93 | intron | SH3BP4 | U |
| 541 | chr9 | 36083406 | 36083628 | Small Intestine Epithelium | 0.08 | 0.92 | exon | RECK | U |
| 542 | chr15 | 72081498 | 72081912 | Small Intestine Epithelium | 0.05 | 0.87 | Intergenic | NR2E3 | U |
| 543 | chr6 | 16178665 | 16178896 | Small Intestine Epithelium | 0.07 | 0.88 | Intergenic | MIR4639 | U |
| 544 | chr13 | 114887133 | 114887370 | Small Intestine Epithelium | 0.1 | 0.91 | intron | RASA3 | U |
| 545 | chr1 | 115184761 | 115184864 | Small Intestine Epithelium | 0.14 | 0.93 | intron | DENND2C | U |
| 546 | chr20 | 31074767 | 31074870 | Small Intestine Epithelium | 0.09 | 0.87 | intron | C20orf112 | U |
| 547 | chr16 | 69630344 | 69630482 | Small Intestine Epithelium | 0.13 | 0.91 | intron | NFAT5 | U |
| 548 | chr15 | 80279940 | 80280171 | Small Intestine Epithelium | 0.06 | 0.84 | Intergenic | BCL2A1 | U |
| 549 | chr22 | 37451029 | 37451265 | Small Intestine Epithelium | 0.14 | 0.9 | intron | KCTD17 | U |
| 550 | chr20 | 11964637 | 11964694 | Small Intestine Epithelium | 0.13 | 0.86 | Intergenic | BTBD3 | U |
| 551 | chr22 | 25080123 | 25080621 | Small Intestine Epithelium | 0.14 | 0.87 | Intergenic | POM121L10P | U |
| 552 | chr7 | 151585286 | 151585413 | Small Intestine Epithelium | 0.03 | 0.74 | Intergenic | PRKAG2-AS1 | U |
| 553 | chr6 | 262568921 | 26257077 | Small Intestine Epithelium | 0.21 | 0.9 | Intergenic | HIST1H2BH | U |
| 554 | chr9 | 36304087 | 35304363 | Small Intestine Epithelium | 0.33 | 0.94 | Intergenic | GNE | U |
| 555 | chr6 | 40996092 | 40996274 | Small Intestine Epithelium | 0.83 | 0.1 | exon | UNC5CL | M |
| 556 | chr1 | 109806335 | 109806380 | Small Intestine Epithelium | 0.73 | 0.13 | exon | CELSR2 | M |
| 557 | chr12 | 13687748 | 13687993 | Small Intestine Epithelium | 0.69 | 0.14 | Intergenic | C12orf36 | M |
| 558 | chr1 | 2430535461 | 243053658 | Small Intestine Epithelium | 0.63 | 0.11 | Intergenic | LOC731275 | M |
| 559 | chr15 | 24722927 | 24722963 | Small Intestine Epithelium | 0.78 | 0.29 | Intergenic | PWRN1 | M |
| 560 | chr19 | 54496256 | 54496354 | Small Intestine Epithelium | 0.69 | 0.21 | exon | CACNG6 | M |
| 561 | chr20 | 2832800 | 2833054 | Small Intestine Epithelium | 0.62 | 0.15 | intron | VPS16 | M |
| 562 | chr12 | 128866366 | 128866525 | Small Intestine Epithelium | 0.65 | 0.2 | intron | TMEM132C | M |
| 563 | chr15 | 24722995 | 24723146 | Small Intestine Epithelium | 0.63 | 0.19 | Intergenic | PWRN1 | M |
| 564 | chr19 | 54496412 | 54496449 | Small Intestine Epithelium | 0.64 | 0.26 | exon | CACNG6 | M |
| 565 | chr20 | 3052397 | 3052413 | Small Intestine Epithelium | 0.79 | 0.21 | exon | OXT | M |
| 566 | chr14 | 21131479 | 21131656 | Small Intestine Epithelium | 0.92 | 0.04 | Intergenic | ANG | M |
| 567 | chr1 | 47904946 | 47905252 | Small Intestine Epithelium | 0.95 | 0.19 | exon | FOXD2 | M |
| 568 | chr14 | 38069639 | 38069729 | Small Intestine Epithelium | 0.83 | 0.08 | Intergenic | FOXA1 | M |
| 569 | chr14 | 38053893 | 38054073 | Small Intestine Epithelium | 0.87 | 0.13 | Intergenic | FOXA1 | M |
| 570 | chr1 | 47904747 | 47904944 | Small Intestine Epithelium | 0.81 | 0.11 | exon | FOXD2 | M |
| 571 | chr9 | 23820835 | 23820911 | Small Intestine Epithelium | 0.79 | 0.11 | intron | ELAVL2 | M |
| 572 | chr20 | 21000837 | 21000920 | Small Intestine Epithelium | 0.81 | 0.13 | Intergenic | PLK1S1 | M |
| 573 | chr14 | 21121138 | 21121275 | Small Intestine Epithelium | 0.76 | 0.1 | Intergenic | OR6S1 | M |
| 574 | chr12 | 133481557 | 133481642 | Small Intestine Epithelium | 0.7 | 0.1 | Intergenic | CHFR | M |
| 575 | chr4 | 188916566 | 188916607 | Small Intestine Epithelium | 0.77 | 0.2 | promoter-TSS | ZFP42 | M |
| 576 | chr2 | 947108 | 947164 | Small Intestine Epithelium | 0.72 | 0.16 | intron | SNTG2 | M |
| 577 | chr1 | 181451403 | 181451508 | Small Intestine Epithelium | 0.65 | 0.11 | Intergenic | CACNA1E | M |
| 578 | chr19 | 29284450 | 29284840 | Small Intestine Epithelium | 0.61 | 0.08 | Intergenic | LOC148145 | M |
| 579 | chr1 | 248021326 | 248021496 | Small Intestine Epithelium | 0.58 | 0.18 | intron | TRIM58 | M |
| 580 | chr15 | 40993737 | 40994201 | Colon Epithelium | 0.02 | 0.94 | exon, intron | RAD51 | U |
| 581 | chr1 | 32781801 | 32781879 | Colon Epithelium | 0.01 | 0.92 | intron | HDAC1 | U |
| 582 | chr6 | 149588777 | 149588812 | Colon Epithelium | 0.02 | 0.92 | Intergenic | TAB2 | U |
| 583 | chr2 | 101641800 | 101641845 | Colon Epithelium | 0.02 | 0.92 | intron | TBC1D8 | U |
| 584 | chr8 | 22101450 | 22101685 | Colon Epithelium | 0.02 | 0.91 | TTS | MIR320A | U |
| 585 | chr12 | 105319208 | 105319364 | Colon Epithelium | 0.06 | 0.95 | intron | SLC41A2 | U |
| 586 | chr1 | 47905855 | 47906212 | Colon Epithelium | 0.01 | 0.89 | exon | FOXD2 | U |
| 587 | chr1 | 167870100 | 167870516 | Colon Epithelium | 0.04 | 0.92 | intron | ADCY10 | U |
| 588 | chr2 | 208037779 | 208038078 | Colon Epithelium | 0.03 | 0.9 | Intergenic | KLF7 | U |
| 589 | chr10 | 105133978 | 105134443 | Colon Epithelium | 0.03 | 0.9 | intron | TAF5 | U |
| 590 | chr17 | 18878792 | 18879282 | Colon Epithelium | 0.07 | 0.93 | intron | FAM83G | U |
| 591 | chr15 | 32993304 | 32993382 | Colon Epithelium | 0.04 | 0.9 | Intergenic | GREM1 | U |
| 592 | chr5 | 80476007 | 80476311 | Colon Epithelium | 0.04 | 0.9 | intron | RASGRF2 | U |
| 593 | chr4 | 9148544 | 9148886 | Colon Epithelium | 0.08 | 0.94 | Intergenic | USP17L10 | U |
| 594 | chr13 | 111093702 | 111093905 | Colon Epithelium | 0.06 | 0.89 | intron | COL4A2 | U |
| 595 | chr5 | 75774174 | 75774414 | Colon Epithelium | 0.11 | 0.94 | intron | IQGAP2 | U |
| 596 | chr12 | 132423352 | 132423537 | Colon Epithelium | 0.24 | 0.95 | intron | PUS1 | U |
| 597 | chr4 | 187629328 | 187629491 | Colon Epithelium | 0.01 | 0.93 | exon | FAT1 | U |
| 598 | chr12 | 92716429 | 92716535 | Colon Epithelium | 0.03 | 0.94 | Intergenic | CLLU1 | U |
| 599 | chr15 | 23037878 | 23037984 | Colon Epithelium | 0.03 | 0.94 | Intergenic | NIPA2 | U |
| 600 | chr7 | 104898593 | 104898641 | Colon Epithelium | 0.03 | 0.93 | intron | SRPK2 | U |
| 601 | chr1 | 32781649 | 32781746 | Colon Epithelium | 0.03 | 0.92 | intron | HDAC1 | U |
| 602 | chr12 | 10803321 | 10803465 | Colon Epithelium | 0.03 | 0.92 | intron | STYK1 | U |
| 603 | chr3 | 73097647 | 73097785 | Colon Epithelium | 0.03 | 0.91 | intron | PPP4R2 | U |
| 604 | chr4 | 1145817843 | 114581990 | Colon Epithelium | 0.06 | 0.94 | intron | CAMK2D | U |
| 605 | chr15 | 1022846163 | 102284756 | Colon Epithelium | 0.03 | 0.9 | Intergenic | TARSL2 | U |
| 606 | chr13 | 66804770 | 66804865 | Colon Epithelium | 0.03 | 0.89 | Intergenic | PCDH9-AS2 | U |
| 607 | chr10 | 6295071 | 6295248 | Colon Epithelium | 0.05 | 0.91 | Intergenic | LOC399715 | U |
| 608 | chr1 | 48167839 | 48168244 | Colon Epithelium | 0.03 | 0.89 | Intergenic | FOXD2 | U |
| 609 | chr13 | 67133641 | 67133729 | Colon Epithelium | 0.06 | 0.91 | intron | PCDH9 | U |
| 610 | chr12 | 77637780 | 77637886 | Colon Epithelium | 0.05 | 0.9 | Intergenic | E2F7 | U |
| 611 | chr15 | 102283250 | 102283505 | Colon Epithelium | 0.03 | 0.88 | Intergenic | TARSL2 | U |
| 612 | chr6 | 149588877 | 149589069 | Colon Epithelium | 0.02 | 0.86 | Intergenic | TAB2 | U |
| 613 | chr2 | 7426795 | 7427018 | Colon Epithelium | 0.04 | 0.88 | Intergenic | LOC100506274 | U |
| 614 | chr15 | 100349643 | 100349873 | Colon Epithelium | 0.04 | 0.88 | Intergenic | DNM1P46 | U |
| 615 | chr8 | 1012317383 | 101232089 | Colon Epithelium | 0.04 | 0.88 | intron | SPAG1 | U |
| 616 | chr16 | 14970553 | 14970957 | Colon Epithelium | 0.05 | 0.89 | exon, intron | NOMO1, NOMO1 | U |
| 617 | chr1 | 47907972 | 47908445 | Colon Epithelium | 0.04 | 0.88 | Intergenic | FOXD2 | U |
| 618 | chr8 | 95645196 | 95645311 | Colon Epithelium | 0.03 | 0.86 | Intergenic | LOC100288748 | U |
| 619 | chr8 | 70448201 | 70448396 | Colon Epithelium | 0.07 | 0.9 | intron | SULF1 | U |
| 620 | chr5 | 1260165883 | 126016863 | Colon Epithelium | 0.05 | 0.88 | Intergenic | C5orf48 | U |
| 621 | chr3 | 32785074 | 32785396 | Colon Epithelium | 0.11 | 0.93 | intron | CNOT10 | U |
| 622 | chr11 | 77389954 | 77390301 | Colon Epithelium | 0.11 | 0.94 | intron | RSF1 | U |
| 623 | chr13 | 102331535 | 102331598 | Colon Epithelium | 0.08 | 0.9 | intron | ITGBLI | U |
| 624 | chr17 | 77006672 | 77006816 | Colon Epithelium | 0.08 | 0.9 | promoter-TSS, Interg [symbol] | CANT1, CANT1 | U |
| 625 | chr8 | 17168987 | 17169164 | Colon Epithelium | 0.08 | 0.9 | exon, intron | MTMR7, MTMR7 | U |
| 626 | chr1 | 226074373 | 226074588 | Colon Epithelium | 0.11 | 0.92 | exon | LEFTY1 | U |
| 627 | chr1 | 17841376 | 17841597 | Colon Epithelium | 0.07 | 0.89 | Intergenic | ARHGEF10L | U |
| 628 | chr21 | 39491396 | 39491798 | Colon Epithelium | 0.09 | 0.91 | intron | DSCR4 | U |
| 629 | chr6 | 88204364 | 88204835 | Colon Epithelium | 0.11 | 0.92 | intron | SLC35A1 | U |
| 630 | chr4 | 165692647 | 165692781 | Colon Epithelium | 0.05 | 0.86 | intron | LOC100505989 | U |
| 631 | chr12 | 131750166 | 131750335 | Colon Epithelium | 0.06 | 0.87 | Intergenic | LOC116437 | U |
| 632 | chr4 | 20637093 | 20637295 | Colon Epithelium | 0.09 | 0.9 | Intergenic | PACRGL | U |
| 633 | chr13 | 74228143 | 74228636 | Colon Epithelium | 0.06 | 0.87 | Intergenic | KLF12 | U |
| 634 | chr3 | 54357008 | 54357101 | Colon Epithelium | 0.07 | 0.87 | intron | CACNA2D3 | U |
| 635 | chr8 | 69769182 | 69769301 | Colon Epithelium | 0.12 | 0.92 | Intergenic | LOC100505718 | U |
| 636 | chr7 | 2086704 | 2086897 | Colon Epithelium | 0.12 | 0.92 | intron | MAD1L1 | U |
| 637 | chr19 | 42213576 | 42213814 | Colon Epithelium | 0.02 | 0.82 | intron, exon | CEACAM5, CEACAM5 | U |
| 638 | chr2 | 240940330 | 240940570 | Colon Epithelium | 0.04 | 0.84 | intron | NDUFA10 | U |
| 639 | chr22 | 44430886 | 44431227 | Colon Epithelium | 0.06 | 0.86 | intron | PARVB | U |
| 640 | chr2 | 204437085 | 204437260 | Colon Epithelium | 0.13 | 0.92 | Intergenic | RAPH1 | U |
| 641 | chr5 | 17324319 | 17324573 | Colon Epithelium | 0.07 | 0.86 | Intergenic | LOC401177 | U |
| 642 | chr2 | 1644300 | 1644764 | Colon Epithelium | 0.09 | 0.87 | intron | PXDN | U |
| 643 | chr15 | 75473667 | 75473948 | Colon Epithelium | 0.14 | 0.91 | Intergenic | C15orf39 | U |
| 644 | chr5 | 158749703 | 158749961 | Colon Epithelium | 0.15 | 0.91 | intron | TULP4 | U |
| 645 | chr6 | 153448780 | 153449113 | Colon Epithelium | 0.08 | 0.84 | intron | RGS17 | U |
| 646 | chr21 | 35362161 | 35362507 | Colon Epithelium | 0.04 | 0.79 | Intergenic | LINC00649 | U |
| 647 | chr2 | 101641230 | 101641660 | Colon Epithelium | 0.14 | 0.89 | intron | TBC1D8 | U |
| 648 | chr3 | 125474843 | 125475095 | Colon Epithelium | 0.17 | 0.91 | Intergenic | MIR548I1 | U |
| 649 | chr3 | 117070041 | 117070439 | Colon Epithelium | 0.18 | 0.91 | Intergenic | LSAMP-AS3 | U |
| 650 | chr6 | 1084535403 | 108454026 | Colon Epithelium | 0.18 | 0.91 | Intergenic | NR2E1 | U |
| 651 | chr16 | 79462842 | 79462977 | Colon Epithelium | 0.19 | 0.91 | Intergenic | MAF | U |
| 652 | chr2 | 69419515 | 69419679 | Colon Epithelium | 0.21 | 0.92 | intron | ANTXR1 | U |
| 653 | chr3 | 122070197 | 122070471 | Colon Epithelium | 0.21 | 0.91 | Intergenic | CSTA | U |
| 654 | chr1 | 224986378 | 224986519 | Colon Epithelium | 0.22 | 0.91 | Intergenic | DNAH14 | U |
| 655 | chr16 | 71461301 | 71461767 | Colon Epithelium | 0.14 | 0.83 | Intergenic | ZNF23 | U |
| 656 | chr6 | 109517041 | 109517347 | Colon Epithelium | 0.28 | 0.9 | Intergenic | CEP57L1 | U |
| 657 | chr7 | 97904665 | 97904988 | Colon Epithelium | 0.31 | 0.92 | Intergenic | BRI3 | U |
| 658 | chr10 | 88636293 | 88636508 | Colon Epithelium | 0.13 | 0.93 | intron | BMPR1A | U |
| 659 | chr1 | 108224264 | 108224525 | Colon Epithelium | 0.13 | 0.91 | intron | VAV3 | U |
| 660 | chr1 | 184567735 | 184568230 | Colon Epithelium | 0.13 | 0.91 | intron | C1orf21 | U |
| 661 | chr10 | 114252502 | 114252719 | Colon Epithelium | 0.03 | 0.96 | intron | VTI1A | U |
| 662 | chr4 | 48679336 | 48679572 | Colon Epithelium | 0.04 | 0.95 | intron | FRYL | U |
| 663 | chr6 | 4909889 | 4910096 | Colon Epithelium | 0.03 | 0.93 | intron | CDYL | U |
| 664 | chr16 | 81453443 | 81453728 | Colon Epithelium | 0.04 | 0.94 | Intergenic | CMIP | U |
| 665 | chr2 | 74323528 | 74323722 | Colon Epithelium | 0.05 | 0.93 | intron | TET3 | U |
| 666 | chr1 | 168057707 | 168058113 | Colon Epithelium | 0.11 | 0.93 | intron | GPR161 | U |
| 667 | chr3 | 195992869 | 195993170 | Colon Epithelium | 0.12 | 0.95 | intron | PCYT1A | U |
| 668 | chr8 | 141594714 | 141595054 | Colon Epithelium | 0.17 | 0.94 | intron | AGO2 | U |
| 669 | chr14 | 62106209 | 62106229 | Colon Epithelium | 0.05 | 0.96 | intron | FLI22447 | U |
| 670 | chr5 | 78264407 | 78264577 | Colon Epithelium | 0.03 | 0.93 | intron | ARSB | U |
| 671 | chr3 | 123667213 | 123667567 | Colon Epithelium | 0.05 | 0.95 | intron | CCDC14 | U |
| 672 | chr3 | 195990693 | 195990795 | Colon Epithelium | 0.06 | 0.95 | intron | PCYT1A | U |
| 673 | chr2 | 242735211 | 242735409 | Colon Epithelium | 0.04 | 0.93 | intron | GAL3ST2 | U |
| 674 | chr18 | 77459409 | 77459848 | Colon Epithelium | 0.05 | 0.94 | intron | CTDP1 | U |
| 675 | chr16 | 18884841 | 18885332 | Colon Epithelium | 0.04 | 0.93 | intron | SMG1 | U |
| 676 | chr16 | 66781322 | 66781542 | Colon Epithelium | 0.03 | 0.91 | intron | DYNC1LI2 | U |
| 677 | chr15 | 933644341 | 93364776 | Colon Epithelium | 0.03 | 0.91 | Intergenic | ASB9P1 | U |
| 678 | chr10 | 861764103 | 86176816 | Colon Epithelium | 0.05 | 0.93 | intron | CCSER2 | U |
| 679 | chr3 | 1578667613 | 157866908 | Colon Epithelium | 0.09 | 0.94 | intron | RSRC1 | U |
| 680 | chr12 | 132423840 | 132424024 | Colon Epithelium | 0.03 | 0.88 | intron | PUS1 | U |
| 681 | chr12 | 89772149 | 89772392 | Colon Epithelium | 0.09 | 0.94 | Intergenic | DUSP6 | U |
| 682 | chr3 | 33689049 | 33689343 | Colon Epithelium | 0.09 | 0.94 | intron | CLASP2 | U |
| 683 | chr3 | 72618758 | 72619029 | Colon Epithelium | 0.13 | 0.94 | Intergenic | RYBP | U |
| 684 | chr6 | 35109390 | 35109499 | Colon Epithelium | 0.1 | 0.93 | promoter-TSS | TCP11 | U |
| 685 | chr7 | 107483770 | 107483936 | Colon Epithelium | 0.09 | 0.92 | Intergenic | SLC26A3 | U |
| 686 | chr2 | 240940021 | 240940299 | Colon Epithelium | 0.09 | 0.92 | intron | NDUFA10 | U |
| 687 | chr11 | 14564373 | 14564657 | Colon Epithelium | 0.11 | 0.94 | intron | PSMA1 | U |
| 688 | chr14 | 74114572 | 74114806 | Colon Epithelium | 0.05 | 0.87 | intron | DNAL1 | U |
| 689 | chr9 | 35248546 | 35248925 | Colon Epithelium | 0.05 | 0.87 | intron | UNC13B | U |
| 690 | chr7 | 65938591 | 65938940 | Colon Epithelium | 0.11 | 0.92 | Intergenic | LINC00174 | U |
| 691 | chr2 | 17830574 | 17830762 | Colon Epithelium | 0.13 | 0.93 | intron | VSNL1 | U |
| 692 | chr20 | 13203947 | 13204216 | Colon Epithelium | 0.14 | 0.93 | intron | ISM1 | U |
| 693 | chr15 | 28365155 | 28365446 | Colon Epithelium | 0.12 | 0.91 | intron | HERC2 | U |
| 694 | chr17 | 27056518 | 27056950 | Colon Epithelium | 0.13 | 0.92 | intron | NEK8 | U |
| 695 | chr7 | 65937750 | 65937991 | Colon Epithelium | 0.17 | 0.94 | Intergenic | LINC00174 | U |
| 696 | chr21 | 33058281 | 33058572 | Colon Epithelium | 0.18 | 0.94 | intron | SCAF4 | U |
| 697 | chr9 | 115603152 | 115603322 | Colon Epithelium | 0.16 | 0.91 | intron | SNX30 | U |
| 698 | chr6 | 135676756 | 135677196 | Colon Epithelium | 0.22 | 0.95 | intron | AHI1 | U |
| 699 | chr14 | 100591192 | 100591341 | Colon Epithelium | 0.19 | 0.91 | intron | EVL | U |
| 700 | chr19 | 56638704 | 56638958 | Colon Epithelium | 0.25 | 0.93 | Intergenic | ZNF787 | U |
| 701 | chr6 | 156909918 | 156910244 | Colon Epithelium | 0.26 | 0.94 | Intergenic | ARID18 | U |
| 702 | chr19 | 14005726 | 14006214 | Colon Epithelium | 0.22 | 0.89 | intron | C19orf57 | U |
| 703 | chr7 | 2102632 | 2103070 | Colon Epithelium | 0.28 | 0.92 | intron | MAD1L1 | U |
| 704 | chr10 | 5786545 | 5786789 | Colon Epithelium | 0.29 | 0.92 | intron | FAM2088 | U |
| 705 | chr2 | 1584538001 | 158453970 | Colon Epithelium | 0.96 | 0.17 | intron | ACVR1C | M |
| 706 | chr3 | 128787334 | 128787628 | Colon Epithelium | 0.88 | 0.09 | Intergenic | GP9 | M |
| 707 | chr1 | 47915410 | 47915504 | Colon Epithelium | 0.88 | 0.1 | Intergenic | FOXD2 | M |
| 708 | chr1 | 53651398 | 53651555 | Colon Epithelium | 0.85 | 0.08 | Intergenic | CPT2 | M |
| 709 | chr4 | 74864368 | 74864615 | Colon Epithelium | 0.92 | 0.15 | promoter-TSS | CXCL5 | M |
| 710 | chr7 | 100659999 | 100660242 | Colon Epithelium | 0.89 | 0.14 | intron | MUC12 | M |
| 711 | chr10 | 27530422 | 27530535 | Colon Epithelium | 0.88 | 0.21 | promoter-TSS | ACBD5 | M |
| 712 | chr9 | 133809106 | 133809362 | Colon Epithelium | 0.79 | 0.16 | intron | FIBCD1 | M |
| 713 | chr17 | 19881550 | 19881704 | Colon Epithelium | 0.68 | 0.06 | promoter-TSS | AKAP10 | M |
| 714 | chr2 | 201829061 | 201829187 | Colon Epithelium | 0.72 | 0.13 | promoter-TSS | ORC2 | M |
| 715 | chr11 | 56981037 | 56981360 | Colon Epithelium | 0.82 | 0.23 | Intergenic | APLNR | M |
| 716 | chr2 | 158453973 | 158454244 | Colon Epithelium | 0.95 | 0.05 | promoter-TSS | ACVR1C | M |
| 717 | chr4 | 74864044 | 74864329 | Colon Epithelium | 0.96 | 0.07 | intron | CXCL5 | M |
| 718 | chr17 | 70215501 | 70215736 | Colon Epithelium | 0.96 | 0.13 | Intergenic | SOX9 | M |
| 719 | chr21 | 43181441 | 43181698 | Colon Epithelium | 0.9 | 0.1 | intron | RIPK4 | M |
| 720 | chr6 | 26233551 | 26233662 | Colon Epithelium | 0.94 | 0.15 | TTS | HIST1H1D | M |
| 721 | chr14 | 102172344 | 102172558 | Colon Epithelium | 0.89 | 0.1 | Intergenic | LINC00239 | M |
| 722 | chr1 | 61509219 | 61509304 | Colon Epithelium | 0.9 | 0.12 | Intergenic | NFIA | M |
| 723 | chr3 | 120169978 | 120170250 | Colon Epithelium | 0.8 | 0.05 | promoter-TSS, intro [symbol] | FSTL1, FSTL1 | M |
| 724 | chr8 | 38645085 | 38645218 | Colon Epithelium | 0.76 | 0.02 | exon | TACC1 | M |
| 725 | chr11 | 9112509 | 9112684 | Colon Epithelium | 0.8 | 0.07 | promoter-TSS | MIR5691 | M |
| 726 | chr20 | 42876648 | 42876760 | Colon Epithelium | 0.72 | 0.05 | intron | GDAP1L1 | M |
| 727 | chr21 | 43181729 | 43182115 | Colon Epithelium | 0.69 | 0.05 | intron | RIPKA | M |
| 728 | chr4 | 11429310 | 11429674 | Colon Epithelium | 0.66 | 0.04 | intron | HS3ST1 | M |
| 729 | chr14 | 21341863 | 21342067 | Colon Epithelium | 0.65 | 0.04 | Intergenic | RNASE3 | M |
| 730 | chr1 | 232423115 | 232423446 | Colon Fibroblasts | 0.26 | 0.85 | Intergenic | SIPA1L2 | U |
| 731 | chr19 | 49961241 | 49961348 | Colon Fibroblasts | 0.33 | 0.84 | intron | ALDH16A1 | U |
| 732 | chr15 | 38716228 | 38716438 | Colon Fibroblasts | 0.36 | 0.87 | Intergenic | FAM98B | U |
| 733 | chr1 | 221065480 | 221065537 | Colon Fibroblasts | 0.67 | 0.18 | Intergenic | HLX | M |
| 734 | chr17 | 32624822 | 32625237 | Colon Fibroblasts | 0.63 | 0.18 | Intergenic | CCL11 | M |
| 735 | chr17 | 46679079 | 46679274 | Colon Fibroblasts | 0.57 | 0.13 | intron | HOXB-AS3 | M |
| 736 | chr4 | 13536606 | 13536659 | Colon Fibroblasts | 0.56 | 0.17 | Intergenic | LOC285547 | M |
| 737 | chr1 | 233751029 | 233751089 | Colon Fibroblasts | 0.58 | 0.19 | intron | KCNK1 | M |
| 738 | chr3 | 134094044 | 134094236 | Colon Fibroblasts | 0.54 | 0.16 | promoter-TSS | AMOTL2 | M |
| 739 | chr14 | 86352363 | 86352431 | Colon Fibroblasts | 0.59 | 0.5 | Intergenic | FLRT2 | M |
| 740 | chr9 | 98514351 | 98514536 | Colon Fibroblasts | 0.64 | 0.22 | Intergenic | ERCC6L2 | M |
| 741 | chr9 | 98514556 | 98514669 | Colon Fibroblasts | 0.66 | 0.28 | Intergenic | ERCC6L2 | M |
| 742 | chr17 | 60217867 | 60217995 | Gallbladder Epithelium | 0.01 | 0.93 | Intergenic | MED13 | U |
| 743 | chr3 | 149198710 | 149198941 | Gallbladder Epithelium | 0.01 | 0.93 | intron | TM4SF4 | U |
| 744 | chr6 | 111272867 | 111273198 | Gallbladder Epithelium | 0.01 | 0.93 | Intergenic | GTF3C6 | U |
| 745 | chr19 | 55738427 | 55738739 | Gallbladder Epithelium | 0.03 | 0.95 | exon | TMEM86B | U |
| 746 | chr1 | 167885483 | 167885593 | Gallbladder Epithelium | 0.05 | 0.96 | TTS | MPC2 | U |
| 747 | chr3 | 171469408 | 171469787 | Gallbladder Epithelium | 0.05 | 0.96 | intron | PLD1 | U |
| 748 | chr3 | 71157777 | 71157870 | Gallbladder Epithelium | 0.08 | 0.97 | intron | FOXP1 | U |
| 749 | chr12 | 121412626 | 121412837 | Gallbladder Epithelium | 0.04 | 0.93 | Intergenic | HNF1A-AS1 | U |
| 750 | chr17 | 60217549 | 60217821 | Gallbladder Epithelium | 0.03 | 0.91 | Intergenic | MED13 | U |
| 751 | chr2 | 171783089 | 171783264 | Gallbladder Epithelium | 0.06 | 0.93 | Intergenic | GORASP2 | U |
| 752 | chr3 | 171463636 | 171463793 | Gallbladder Epithelium | 0.08 | 0.95 | intron | PLD1 | U |
| 753 | chr19 | 55738780 | 55739169 | Gallbladder Epithelium | 0.01 | 0.88 | exon, intron | TMEM86B | U |
| 754 | chr3 | 141737928 | 141738402 | Gallbladder Epithelium | 0.07 | 0.94 | intron | TFDP2 | U |
| 755 | chr11 | 1243400 | 1243605 | Gallbladder Epithelium | 0.03 | 0.89 | promoter-TSS | MUC5B | U |
| 756 | chr13 | 41833664 | 41833902 | Gallbladder Epithelium | 0.11 | 0.95 | intron | MTRF1 | U |
| 757 | chr19 | 55742007 | 55742487 | Gallbladder Epithelium | 0.07 | 0.89 | exon | PPP6R1 | U |
| 758 | chr19 | 16475134 | 16475347 | Gallbladder Epithelium | 0.14 | 0.93 | intron | EPS15L1 | U |
| 759 | chr16 | 72996787 | 72996887 | Gallbladder Epithelium | 0.01 | 0.94 | intron | ZFHX3 | U |
| 760 | chr3 | 171463829 | 171464225 | Gallbladder Epithelium | 0.02 | 0.95 | intron | PLD1 | U |
| 761 | chr5 | 149238943 | 149239195 | Gallbladder Epithelium | 0.02 | 0.93 | intron | UST | U |
| 762 | chr2 | 232263444 | 232263922 | Gallbladder Epithelium | 0.03 | 0.94 | exon | B3GNT7 | U |
| 763 | chr10 | 111724743 | 111724869 | Gallbladder Epithelium | 0.03 | 0.93 | intron | LOC100505933 | U |
| 764 | chr1 | 26188334 | 26188494 | Gallbladder Epithelium | 0.02 | 0.92 | exon | PAQR7 | U |
| 765 | chr18 | 4002742 | 4002925 | Gallbladder Epithelium | 0.01 | 0.91 | intron | DLGAP1 | U |
| 766 | chr3 | 171463730 | 171463794 | Gallbladder Epithelium | 0.08 | 0.97 | intron | PLD1 | U |
| 767 | chr10 | 108487813 | 108487937 | Gallbladder Epithelium | 0.02 | 0.91 | intron | SORCS1 | U |
| 768 | chr18 | 60670121 | 60670254 | Gallbladder Epithelium | 0.02 | 0.91 | Intergenic | PHLPP1 | U |
| 769 | chr4 | 91455381 | 91455570 | Gallbladder Epithelium | 0.03 | 0.92 | intron | CCSER1 | U |
| 770 | chr10 | 4161365 | 4161581 | Gallbladder Epithelium | 0.05 | 0.94 | Intergenic | KLF6 | U |
| 771 | chr15 | 96990472 | 96990904 | Gallbladder Epithelium | 0.02 | 0.91 | Intergenic | MIR1469 | U |
| 772 | chr11 | 1249946 | 1250054 | Gallbladder Epithelium | 0 | 0.88 | intron | MUC5B | U |
| 773 | chr7 | 156286975 | 156287090 | Gallbladder Epithelium | 0.03 | 0.91 | Intergenic | LINC00244 | U |
| 774 | chr12 | 93502354 | 93502725 | Gallbladder Epithelium | 0.01 | 0.89 | intron | LOC643339 | U |
| 775 | chr19 | 1293440 | 1293583 | Gallbladder Epithelium | 0.03 | 0.9 | intron | EFNA2 | U |
| 776 | chr2 | 365054 | 365353 | Gallbladder Epithelium | 0.04 | 0.91 | Intergenic | FAM150B | U |
| 777 | chr18 | 60672273 | 60672600 | Gallbladder Epithelium | 0.02 | 0.89 | Intergenic | PHLPP1 | U |
| 778 | chr19 | 40472611 | 40473010 | Gallbladder Epithelium | 0.02 | 0.89 | Intergenic | PSMC4 | U |
| 779 | chr13 | 24447224 | 24447392 | Gallbladder Epithelium | 0.03 | 0.89 | intron | MIPEP | U |
| 780 | chr17 | 16304390 | 16304570 | Gallbladder Epithelium | 0.03 | 0.89 | Intergenic | TRPV2 | U |
| 781 | chr10 | 12452740 | 12452958 | Gallbladder Epithelium | 0.05 | 0.91 | intron | CAMK1D | U |
| 782 | chr2 | 54918167 | 54918417 | Gallbladder Epithelium | 0.05 | 0.91 | Intergenic | EML6 | U |
| 783 | chr20 | 56510607 | 56510857 | Gallbladder Epithelium | 0.02 | 0.88 | Intergenic | MIR4532 | U |
| 784 | chr11 | 120407149 | 120407564 | Gallbladder Epithelium | 0.04 | 0.9 | Intergenic | GRIK4 | U |
| 785 | chr1 | 19704327 | 19704536 | Gallbladder Epithelium | 0.07 | 0.92 | intron | CAPZB | U |
| 786 | chr3 | 101820487 | 101820732 | Gallbladder Epithelium | 0.08 | 0.93 | Intergenic | LOC152225 | U |
| 787 | chr15 | 59567074 | 59567402 | Gallbladder Epithelium | 0.05 | 0.9 | intron | MYO1E | U |
| 788 | chr17 | 73524013 | 73524358 | Gallbladder Epithelium | 0.05 | 0.9 | intron | LLGL2 | U |
| 789 | chr6 | 158482805 | 158483162 | Gallbladder Epithelium | 0.05 | 0.9 | intron | SYNJ2 | U |
| 790 | chr8 | 28491963 | 28492355 | Gallbladder Epithelium | 0.07 | 0.92 | Intergenic | EXTL3 | U |
| 791 | chr18 | 60667371 | 60667796 | Gallbladder Epithelium | 0.08 | 0.93 | Intergenic | PHLPP1 | U |
| 792 | chr8 | 29986696 | 29986949 | Gallbladder Epithelium | 0.06 | 0.9 | intron | LEPROTL1 | U |
| 793 | chr16 | 48449938 | 48450248 | Gallbladder Epithelium | 0.05 | 0.89 | Intergenic | SIAH1 | U |
| 794 | chr11 | 1247043 | 1247202 | Gallbladder Epithelium | 0.04 | 0.87 | intron | MUC5B | U |
| 795 | chr4 | 100907155 | 100907416 | Gallbladder Epithelium | 0.05 | 0.88 | Intergenic | LOC256880 | U |
| 796 | chr13 | 107388914 | 107389195 | Gallbladder Epithelium | 0.05 | 0.88 | Intergenic | LINC00443 | U |
| 797 | chr12 | 121413009 | 121413450 | Gallbladder Epithelium | 0.09 | 0.92 | Intergenic | HNF1A-AS1 | U |
| 798 | chr2 | 235857897 | 235857967 | Gallbladder Epithelium | 0.12 | 0.94 | Intergenic | SH38P4 | U |
| 799 | chr21 | 44880700 | 44880922 | Gallbladder Epithelium | 0.07 | 0.89 | Intergenic | LINC00319 | U |
| 800 | chr17 | 360880543 | 36088321 | Gallbladder Epithelium | 0.09 | 0.91 | intron | HNF1B | U |
| 801 | chr8 | 29987038 | 29987389 | Gallbladder Epithelium | 0.05 | 0.87 | intron | LEPROTL1 | U |
| 802 | chr16 | 81819407 | 81819798 | Gallbladder Epithelium | 0.09 | 0.91 | intron, exon | PLCG2, PLCG2 | U |
| 803 | chr17 | 79914295 | 79914394 | Gallbladder Epithelium | 0.13 | 0.91 | intron | NOTUM | U |
| 804 | chr11 | 39868461 | 39868570 | Gallbladder Epithelium | 0.11 | 0.92 | Intergenic | LRRC4C | U |
| 805 | chr12 | 100969290 | 100969416 | Gallbladder Epithelium | 0.14 | 0.95 | intron | GAS2L3 | U |
| 806 | chr5 | 111559671 | 111559810 | Gallbladder Epithelium | 0.13 | 0.94 | intron | EPB41L4A | U |
| 807 | chr11 | 39868259 | 39868438 | Gallbladder Epithelium | 0.11 | 0.92 | Intergenic | LRRCAC | U |
| 808 | chr3 | 195071885 | 195072093 | Gallbladder Epithelium | 0.12 | 0.92 | intron | ACAP2 | U |
| 809 | chr15 | 85400424 | 85400643 | Gallbladder Epithelium | 0.09 | 0.89 | exon | ALPK3 | U |
| 810 | chr19 | 55717579 | 55717841 | Gallbladder Epithelium | 0.06 | 0.86 | intron | PTPRH | U |
| 811 | chr9 | 116673851 | 116674325 | Gallbladder Epithelium | 0.11 | 0.91 | intron | ZNF618 | U |
| 812 | chr11 | 60527423 | 60527466 | Gallbladder Epithelium | 0.09 | 0.88 | intron | MS4A15 | U |
| 813 | chr15 | 70929583 | 70929648 | Gallbladder Epithelium | 0.11 | 0.89 | Intergenic | UACA | U |
| 814 | chr12 | 50955346 | 50955659 | Gallbladder Epithelium | 0.14 | 0.93 | intron | DIP2B | U |
| 815 | chr1 | 16444223 | 16444600 | Gallbladder Epithelium | 0.13 | 0.92 | Intergenic | EPHA2 | U |
| 816 | chr11 | 64790716 | 64790825 | Gallbladder Epithelium | 0.11 | 0.89 | intron | ARL2-SNX15 | U |
| 817 | chr16 | 57638222 | 57638522 | Gallbladder Epithelium | 0.16 | 0.92 | Intergenic | GPR56 | U |
| 818 | chr21 | 44838717 | 44838851 | Gallbladder Epithelium | 0.11 | 0.86 | intron | SIK1 | U |
| 819 | chr18 | 3176288 | 3176650 | Gallbladder Epithelium | 0.15 | 0.9 | intron | MYOM1 | U |
| 820 | chr11 | 65607111 | 65607368 | Gallbladder Epithelium | 0.21 | 0.95 | intron | SNX32 | U |
| 821 | chr5 | 131961615 | 131961840 | Gallbladder Epithelium | 0.19 | 0.92 | intron | RAD50 | U |
| 822 | chr11 | 1862243 | 1862442 | Gallbladder Epithelium | 0.19 | 0.91 | exon | TNNI2 | U |
| 823 | chr3 | 118961606 | 118961918 | Gallbladder Epithelium | 0.17 | 0.89 | Intergenic | B4GALT4 | U |
| 824 | chr2 | 3205853 | 3206004 | Gallbladder Epithelium | 0.24 | 0.95 | intron | TSSC1 | U |
| 825 | chr19 | 2172333 | 2172590 | Gallbladder Epithelium | 0.2 | 0.91 | intron | DOT1L | U |
| 826 | chr17 | 16187347 | 16187583 | Gallbladder Epithelium | 0.25 | 0.95 | intron | PIGL | U |
| 827 | chr4 | 111004479 | 111004907 | Gallbladder Epithelium | 0.27 | 0.96 | intron | ELOVL6 | U |
| 828 | chr7 | 30824632 | 30825029 | Gallbladder Epithelium | 0.34 | 0.96 | intron | INMT-FAM188B | U |
| 829 | chr4 | 1306280 | 1306521 | Gallbladder Epithelium | 0.33 | 0.94 | intron | MAEA | U |
| 830 | chr2 | 223540221 | 223540662 | Gallbladder Epithelium | 0.15 | 0.92 | intron | MOGAT1 | U |
| 831 | chr20 | 53260018 | 53260192 | Gallbladder Epithelium | 0.16 | 0.91 | intron | DOK5 | U |
| 832 | chr18 | 3214677 | 3215154 | Gallbladder Epithelium | 0.01 | 0.95 | intron | MYOM1 | U |
| 833 | chr14 | 51491780 | 51492061 | Gallbladder Epithelium | 0.01 | 0.93 | intron | TRIM9 | U |
| 834 | chr11 | 1249809 | 1249896 | Gallbladder Epithelium | 0.03 | 0.94 | exon | MUC5B | U |
| 835 | chr21 | 44838854 | 44839324 | Gallbladder Epithelium | 0.04 | 0.94 | exon, intron | SIK1 | U |
| 836 | chr6 | 43752334 | 43752522 | Gallbladder Epithelium | 0.04 | 0.93 | exon | VEGFA | U |
| 837 | chr14 | 64107269 | 64107372 | Gallbladder Epithelium | 0.07 | 0.94 | intron | WDR89 | U |
| 838 | chr8 | 90768568 | 90768800 | Gallbladder Epithelium | 0.07 | 0.94 | Intergenic | RIPK2 | U |
| 839 | chr8 | 90768318 | 90768562 | Gallbladder Epithelium | 0.09 | 0.95 | Intergenic | RIPK2 | U |
| 840 | chr4 | 57082657 | 57083116 | Gallbladder Epithelium | 0.03 | 0.93 | intron | KIAA1211 | U |
| 841 | chr2 | 60642332 | 60642793 | Gallbladder Epithelium | 0.02 | 0.92 | Intergenic | MIR4432 | U |
| 842 | chr10 | 6781345 | 6781605 | Gallbladder Epithelium | 0.04 | 0.93 | Intergenic | LINC00707 | U |
| 843 | chr20 | 36008159 | 36008256 | Gallbladder Epithelium | 0.04 | 0.92 | intron | SRC | U |
| 844 | chr9 | 92976862 | 92976981 | Gallbladder Epithelium | 0 | 0.88 | Intergenic | LOC286370 | U |
| 845 | chr14 | 23748661 | 23749018 | Gallbladder Epithelium | 0.04 | 0.92 | intron | HOMEZ | U |
| 846 | chr14 | 64106921 | 64107174 | Gallbladder Epithelium | 0.05 | 0.92 | intron | WDR89 | U |
| 847 | chr1 | 220012556 | 220012880 | Gallbladder Epithelium | 0.05 | 0.92 | Intergenic | RNU5F-1 | U |
| 848 | chr5 | 500073 | 500481 | Gallbladder Epithelium | 0.04 | 0.91 | intron | SLC9A3 | U |
| 849 | chr11 | 64784378 | 64784793 | Gallbladder Epithelium | 0.06 | 0.92 | intron | ARL2-SNX15 | U |
| 850 | chr22 | 37971997 | 37972416 | Gallbladder Epithelium | 0.03 | 0.89 | intron | LGALS2 | U |
| 851 | chr19 | 55740650 | 55740846 | Gallbladder Epithelium | 0.09 | 0.94 | TTS, promoter-TSS | PPP6R1, TMEM868 | U |
| 852 | chr8 | 117667019 | 117667358 | Gallbladder Epithelium | 0.04 | 0.89 | intron | EIF3H | U |
| 853 | chr5 | 1161093 | 1161312 | Gallbladder Epithelium | 0.09 | 0.93 | Intergenic | SLC6A19 | U |
| 854 | chr7 | 1586104 | 1586158 | Gallbladder Epithelium | 0.05 | 0.87 | exon | TMEM184A | U |
| 855 | chr5 | 600978 | 601209 | Gallbladder Epithelium | 0.07 | 0.89 | Intergenic | CEP72 | U |
| 856 | chr6 | 18338103 | 18338416 | Gallbladder Epithelium | 0.03 | 0.85 | Intergenic | RNF1448 | U |
| 857 | chr19 | 53965476 | 53965785 | Gallbladder Epithelium | 0.12 | 0.93 | Intergenic | ZNF813 | U |
| 858 | chr3 | 193586951 | 193587241 | Gallbladder Epithelium | 0.13 | 0.91 | Intergenic | DPPA2P3 | U |
| 859 | chr17 | 77919294 | 77919595 | Gallbladder Epithelium | 0.16 | 0.92 | intron | TBC1D16 | U |
| 860 | chr12 | 95561915 | 95562388 | Gallbladder Epithelium | 0.17 | 0.93 | intron | FGD6 | U |
| 861 | chr11 | 1273472 | 1273921 | Gallbladder Epithelium | 0.15 | 0.9 | exon | MUC5B | U |
| 862 | chr22 | 40578030 | 40578244 | Gallbladder Epithelium | 0.25 | 0.93 | intron | TNRC6B | U |
| 863 | chr6 | 34870056 | 34870450 | Gallbladder Epithelium | 0.26 | 0.93 | intron | ANKS1A | U |
| 864 | chr9 | 97661527 | 97561987 | Gallbladder Epithelium | 0.27 | 0.94 | intron | C9orf3 | U |
| 865 | chr7 | 2067755 | 2068235 | Gallbladder Epithelium | 0.31 | 0.96 | intron | MAD1L1 | U |
| 866 | chr5 | 78881963 | 78882210 | Gallbladder Epithelium | 0.33 | 0.93 | Intergenic | PAPD4 | U |
| 867 | chr7 | 895051 | 895511 | Gallbladder Epithelium | 0.36 | 0.95 | intron | SUN1 | U |
| 868 | chr7 | 1013981941 | 101398294 | Gallbladder Epithelium | 0.73 | 0.14 | Intergenic | CUX1 | M |
| 869 | chr12 | 75874414 | 75874745 | Gallbladder Epithelium | 0.64 | 0.11 | promoter-TSS | GLIPR1 | M |
| 870 | chr12 | 111708144 | 111708282 | Gallbladder Epithelium | 0.67 | 0.18 | intron | CUX2 | M |
| 871 | chr14 | 104390340 | 104390644 | Gallbladder Epithelium | 0.5 | 0.13 | Intergenic | C14orf2 | M |
| 872 | chr14 | 106060773 | 106060939 | Gallbladder Epithelium | 0.9 | 0.55 | Intergenic | TMEM121 | M |
| 873 | chr4 | 46994535 | 46994749 | Gallbladder Epithelium | 0.52 | 0.17 | intron | GABRA4 | M |
| 874 | chr10 | 17672985 | 17673386 | Gallbladder Epithelium | 0.89 | 0.56 | intron | STAM | M |
| 875 | chr15 | 30537881 | 30537958 | Gallbladder Epithelium | 0.84 | 0.7 | Intergenic | DKFZP434L187 | M |
| 876 | chr9 | 65862749 | 65863117 | Gallbladder Epithelium | 0.87 | 0.74 | Intergenic | SPATA31AS | M |
| 877 | chr12 | 100869606 | 100869803 | Liver Hepatocytes | 0.02 | 0.93 | intron | NR1H4 | U |
| 878 | chr1 | 9614272 | 9614520 | Liver Hepatocytes | 0.04 | 0.95 | intron | SLC25A33 | U |
| 879 | chr15 | 42940421 | 42940515 | Liver Hepatocytes | 0.04 | 0.94 | intron | STARD9 | U |
| 880 | chr2 | 231385134 | 231385377 | Liver Hepatocytes | 0.04 | 0.93 | intron | SP100 | U |
| 881 | chr16 | 78590461 | 78590725 | Liver Hepatocytes | 0.06 | 0.95 | intron | WWOX | U |
| 882 | chr4 | 110434478 | 110434727 | Liver Hepatocytes | 0.08 | 0.96 | intron | SEC248 | U |
| 883 | chr4 | 31059101 | 3106020 | Liver Hepatocytes | 0.06 | 0.94 | intron | HTT | U |
| 884 | chr15 | 91194697 | 91194833 | Liver Hepatocytes | 0.06 | 0.94 | Intergenic | BLM | U |
| 885 | chr2 | 230936371 | 230936463 | Liver Hepatocytes | 0.06 | 0.93 | Intergenic | SLC16A14 | U |
| 886 | chr10 | 99062257 | 99062593 | Liver Hepatocytes | 0.07 | 0.93 | Intergenic | ARHGAP19 | U |
| 887 | chr17 | 1648769 | 1649109 | Liver Hepatocytes | 0.06 | 0.92 | intron | SERPINF2 | U |
| 888 | chr17 | 79984007 | 79984371 | Liver Hepatocytes | 0.07 | 0.93 | intron | LRRC45 | U |
| 889 | chr9 | 133474347 | 133474506 | Liver Hepatocytes | 0.08 | 0.94 | intron | FUBP3 | U |
| 890 | chr16 | 58625432 | 58625523 | Liver Hepatocytes | 0.11 | 0.96 | intron | CNOT1 | U |
| 891 | chr19 | 45449520 | 45450010 | Liver Hepatocytes | 0.05 | 0.9 | TTS, intron | APOC4, APOC4-APOC [symbol] | U |
| 892 | chr6 | 37114490 | 37114947 | Liver Hepatocytes | 0.1 | 0.94 | Intergenic | PIM1 | U |
| 893 | chr3 | 52245330 | 52245716 | Liver Hepatocytes | 0.12 | 0.95 | exon | ALAS1 | U |
| 894 | chr17 | 80440933 | 80441176 | Liver Hepatocytes | 0.12 | 0.93 | exon | NARF | U |
| 895 | chr16 | 67805212 | 67805663 | Liver Hepatocytes | 0.14 | 0.95 | intron | RANBP10 | U |
| 896 | chr15 | 40959187 | 40959312 | Liver Hepatocytes | 0.15 | 0.95 | Intergenic | LOC100505648 | U |
| 897 | chr4 | 74271468 | 74271601 | Liver Hepatocytes | 0.03 | 0.94 | intron | ALB | U |
| 898 | chr6 | 142606522 | 142606678 | Liver Hepatocytes | 0.05 | 0.95 | Intergenic | GPR126 | U |
| 899 | chr11 | 116705312 | 116705514 | Liver Hepatocytes | 0.04 | 0.94 | Intergenic | APOA1 | U |
| 900 | chr19 | 45792169 | 45792444 | Liver Hepatocytes | 0.05 | 0.94 | intron | MARK4 | U |
| 901 | chr5 | 36403138 | 36403266 | Liver Hepatocytes | 0.04 | 0.92 | Intergenic | RANBP31 | U |
| 902 | chr9 | 128347842 | 128348213 | Liver Hepatocytes | 0.07 | 0.95 | intron | MAPKAP1 | U |
| 903 | chr18 | 56112759 | 56113188 | Liver Hepatocytes | 0.05 | 0.93 | Intergenic | MIR122 | U |
| 904 | chr11 | 132221034 | 132221099 | Liver Hepatocytes | 0.06 | 0.93 | Intergenic | NTM | U |
| 905 | chr1 | 207263326 | 207263501 | Liver Hepatocytes | 0.05 | 0.92 | intron | C4BPB | U |
| 906 | chr12 | 53571394 | 53571569 | Liver Hepatocytes | 0.04 | 0.91 | intron | CSAD | U |
| 907 | chr2 | 121239392 | 121239693 | Liver Hepatocytes | 0.05 | 0.92 | Intergenic | LOC84931 | U |
| 908 | chr1 | 173883833 | 173884207 | Liver Hepatocytes | 0.06 | 0.93 | exon | SERPINC1 | U |
| 909 | chr2 | 119980538 | 119980922 | Liver Hepatocytes | 0.06 | 0.93 | promoter-TSS | STEAP3 | U |
| 910 | chr12 | 53564117 | 53564574 | Liver Hepatocytes | 0.00 | 0.93 | exon, intron | CSAD, CSAD | U |
| 911 | chr10 | 54534928 | 54535120 | Liver Hepatocytes | 0.05 | 0.91 | Intergenic | MBL2 | U |
| 912 | chr6 | 163617164 | 163617399 | Liver Hepatocytes | 0.06 | 0.92 | intron | PACRG | U |
| 913 | chr13 | 34183496 | 34183773 | Liver Hepatocytes | 0.07 | 0.93 | intron | STARD13 | U |
| 914 | chr19 | 27908201 | 2791240 | Liver Hepatocytes | 0.06 | 0.92 | intron | THOP1 | U |
| 915 | chr11 | 116680030 | 116680133 | Liver Hepatocytes | 0.13 | 0.95 | Intergenic | APOA4 | U |
| 916 | chr10 | 91168996 | 91169173 | Liver Hepatocytes | 0.07 | 0.92 | Intergenic | IFITS | U |
| 917 | chr5 | 32119151 | 32119334 | Liver Hepatocytes | 0.07 | 0.92 | Intergenic | GOLPH3 | U |
| 918 | chr11 | 116697148 | 116697339 | Liver Hepatocytes | 0.06 | 0.91 | Intergenic | APOA4 | U |
| 919 | chr1 | 243619235 | 243619436 | Liver Hepatocytes | 0.06 | 0.91 | intron | SDCCAG8 | U |
| 920 | chr1 | 93401410 | 93401622 | Liver Hepatocytes | 0.07 | 0.92 | intron | FAM69A | U |
| 921 | chr17 | 38700428 | 38700643 | Liver Hepatocytes | 0.07 | 0.92 | Intergenic | CCR7 | U |
| 922 | chr7 | 100163609 | 100163830 | Liver Hepatocytes | 0.06 | 0.91 | exon | AGFG2 | U |
| 923 | chr13 | 460152501 | 46015477 | Liver Hepatocytes | 0.05 | 0.9 | Intergenic | SLC25A30 | U |
| 924 | chr19 | 33767237 | 33767466 | Liver Hepatocytes | 0.07 | 0.92 | Intergenic | CEBPA | U |
| 925 | chr18 | 21641760 | 21641992 | Liver Hepatocytes | 0.06 | 0.91 | intron | TTC39C | U |
| 926 | chr8 | 13083273 | 13083574 | Liver Hepatocytes | 0.1 | 0.95 | intron | DLC1 | U |
| 927 | chr21 | 34677555 | 34677877 | Liver Hepatocytes | 0.07 | 0.92 | Intergenic | IFNAR1 | U |
| 928 | chr4 | 39435732 | 39436055 | Liver Hepatocytes | 0.03 | 0.88 | exon | KLB | U |
| 929 | chr17 | 17485333 | 17485733 | Liver Hepatocytes | 0.04 | 0.89 | exon, intron | PEMT, PEMT | U |
| 930 | chr22 | 46528295 | 46528709 | Liver Hepatocytes | 0.06 | 0.91 | Intergenic | PPARA | U |
| 931 | chr1 | 161207275 | 161207712 | Liver Hepatocytes | 0.08 | 0.93 | intron | NR1I3 | U |
| 932 | chr2 | 110069888 | 110070386 | Liver Hepatocytes | 0.05 | 0.9 | intron | SH3RF3 | U |
| 933 | chr6 | 49493139 | 49493240 | Liver Hepatocytes | 0.07 | 0.91 | intron | GLYATL3 | U |
| 934 | chr11 | 354147 | 354264 | Liver Hepatocytes | 0.04 | 0.88 | Intergenic | B4GALNT4 | U |
| 935 | chr4 | 39561899 | 39562084 | Liver Hepatocytes | 0.1 | 0.94 | intron | SMIM14 | U |
| 936 | chr1 | 11106576 | 11106783 | Liver Hepatocytes | 0.06 | 0.9 | exon | MASP2 | U |
| 937 | chr1 | 12585207 | 12585432 | Liver Hepatocytes | 0.06 | 0.9 | Intergenic | SNORA59B | U |
| 938 | chr16 | 72089070 | 72089441 | Liver Hepatocytes | 0.07 | 0.91 | intron | HP | U |
| 939 | chr3 | 50244816 | 50245293 | Liver Hepatocytes | 0.08 | 0.92 | intron | SLC38A3 | U |
| 940 | chr18 | 47360963 | 47361134 | Liver Hepatocytes | 0.08 | 0.91 | intron | MYO5B | U |
| 941 | chr19 | 58864286 | 58864529 | Liver Hepatocytes | 0.05 | 0.88 | exon | A18G | U |
| 942 | chr10 | 134158895 | 134159201 | Liver Hepatocytes | 0.08 | 0.91 | intron | LRRC27 | U |
| 943 | chr19 | 33990040 | 33990380 | Liver Hepatocytes | 0.08 | 0.91 | intron | PEPD | U |
| 944 | chr1 | 12582917 | 12583313 | Liver Hepatocytes | 0.11 | 0.94 | Intergenic | SNORA59B | U |
| 945 | chr19 | 8487337 | 8487567 | Liver Hepatocytes | 0.13 | 0.95 | intron | MARCH2 | U |
| 946 | chr4 | 185272821 | 185273279 | Liver Hepatocytes | 0.11 | 0.93 | intron | LOC728175 | U |
| 947 | chr11 | 77879061 | 77879556 | Liver Hepatocytes | 0.09 | 0.91 | Intergenic | KCTD21 | U |
| 948 | chr15 | 31450558 | 31450613 | Liver Hepatocytes | 0.13 | 0.94 | intron | TRPM1 | U |
| 949 | chr19 | 13303265 | 13303395 | Liver Hepatocytes | 0.09 | 0.9 | Intergenic | IER2 | U |
| 950 | chr3 | 148942102 | 148942284 | Liver Hepatocytes | 0.11 | 0.92 | Intergenic | CP | U |
| 951 | chr15 | 40674143 | 40674362 | Liver Hepatocytes | 0.13 | 0.94 | promoter-TSS | KNSTRN | U |
| 952 | chr16 | 83971779 | 83972003 | Liver Hepatocytes | 0.07 | 0.88 | Intergenic | OSGIN1 | U |
| 953 | chr19 | 49332720 | 49332964 | Liver Hepatocytes | 0.12 | 0.93 | intron | HSD17B14 | U |
| 954 | chr16 | 72096449 | 72096839 | Liver Hepatocytes | 0.06 | 0.87 | promoter-TSS | HPR | U |
| 955 | chr3 | 725247313 | 72525183 | Liver Hepatocytes | 0.09 | 0.9 | Intergenic | RYBP | U |
| 956 | chr4 | 1747210 | 1747291 | Liver Hepatocytes | 0.1 | 0.9 | TTS | TACC3 | U |
| 957 | chr1 | 8019114 | 8019208 | Liver Hepatocytes | 0.11 | 0.91 | Intergenic | PARK7 | U |
| 958 | chr7 | 71149382 | 71149485 | Liver Hepatocytes | 0.11 | 0.91 | intron | WBSCR17 | U |
| 959 | chr15 | 91425594 | 91425800 | Liver Hepatocytes | 0.08 | 0.88 | exon | FURIN | U |
| 960 | chr12 | 1096392601 | 109639506 | Liver Hepatocytes | 0.15 | 0.95 | exon | ACAC8 | U |
| 961 | chr14 | 1035737623 | 103574056 | Liver Hepatocytes | 0.14 | 0.94 | exon, intron | EXOC3L4, EXOC3L4 | U |
| 962 | chr4 | 1746776 | 1747101 | Liver Hepatocytes | 0.1 | 0.9 | TTS | TACC3 | U |
| 963 | chr3 | 1863335633 | 186333911 | Liver Hepatocytes | 0.13 | 0.9 | intron | AHSG | U |
| 964 | chr16 | 58751782 | 58752166 | Liver Hepatocytes | 0.13 | 0.93 | intron | GOT2 | U |
| 965 | chr8 | 22033325 | 22033533 | Liver Hepatocytes | 0.12 | 0.91 | intron | BMP1 | U |
| 966 | chr4 | 6923492 | 6923718 | Liver Hepatocytes | 0.14 | 0.93 | intron | TBC1D14 | U |
| 967 | chr3 | 126237039 | 126237489 | Liver Hepatocytes | 0.13 | 0.89 | promoter-TSS | UROC1 | U |
| 968 | chr11 | 57364364 | 57364499 | Liver Hepatocytes | 0.09 | 0.87 | promoter-TSS | SERPING1 | U |
| 969 | chr6 | 135444057 | 135444239 | Liver Hepatocytes | 0.08 | 0.86 | Intergenic | MYB | U |
| 970 | chr20 | 62254767 | 62255014 | Liver Hepatocytes | 0.14 | 0.92 | intron | GMEB2 | U |
| 971 | chr11 | 610913 | 611184 | Liver Hepatocytes | 0.14 | 0.92 | intron, exon | PHRF1, PHRF1 | U |
| 972 | chr1 | 1700329 | 1700815 | Liver Hepatocytes | 0.17 | 0.95 | intron | NADK | U |
| 973 | chr19 | 45438564 | 45438754 | Liver Hepatocytes | 0.14 | 0.91 | Intergenic | APOC4 | U |
| 974 | chr19 | 4003380 | 4003560 | Liver Hepatocytes | 0.14 | 0.9 | Intergenic | PIAS4 | U |
| 975 | chr2 | 128539850 | 128540067 | Liver Hepatocytes | 0.18 | 0.94 | intron | WDR33 | U |
| 976 | chr12 | 112014821 | 112015139 | Liver Hepatocytes | 0.19 | 0.95 | intron | ATXN2 | U |
| 977 | chr17 | 39843528 | 39843890 | Liver Hepatocytes | 0.12 | 0.88 | Intergenic | EIF1 | U |
| 978 | chr19 | 5871274 | 5871660 | Liver Hepatocytes | 0.13 | 0.89 | promoter-TSS, Interg [symbol] | FUT5, FUT5 | U |
| 979 | chr12 | 122313379 | 122313647 | Liver Hepatocytes | 0.18 | 0.9 | intron | HPD | U |
| 980 | chr8 | 21858621 | 21858815 | Liver Hepatocytes | 0.35 | 0.95 | intron | XPO7 | U |
| 981 | chr6 | 134297765 | 134297908 | Liver Hepatocytes | 0.05 | 0.96 | intron | TBPL1 | U |
| 982 | chr6 | 160500588 | 160501051 | Liver Hepatocytes | 0.04 | 0.95 | intron | IGF2R | U |
| 983 | chr19 | 11347513 | 11347633 | Liver Hepatocytes | 0.04 | 0.94 | exon, intron | DOCK6 | U |
| 984 | chr22 | 50644655 | 50644958 | Liver Hepatocytes | 0.06 | 0.93 | exon, intron | SELO, SELO | U |
| 985 | chr3 | 528125201 | 52812612 | Liver Hepatocytes | 0.05 | 0.89 | promoter-TSS | ITIH1 | U |
| 986 | chr1 | 65376254 | 65376644 | Liver Hepatocytes | 0.1 | 0.93 | intron | JAK1 | U |
| 987 | chr22 | 18214291 | 18214567 | Liver Hepatocytes | 0.06 | 0.92 | TTS | BCL2L13 | U |
| 988 | chr14 | 73628524 | 73628843 | Liver Hepatocytes | 0.11 | 0.96 | intron | PSEN1 | U |
| 989 | chr22 | 21128772 | 21129237 | Liver Hepatocytes | 0.05 | 0.94 | intron | SERPIND1 | U |
| 990 | chr6 | 25930165 | 25930293 | Liver Hepatocytes | 0.09 | 0.96 | intron | SLC17A2 | U |
| 991 | chr9 | 108180324 | 108180552 | Liver Hepatocytes | 0.05 | 0.92 | Intergenic | FSD1L | U |
| 992 | chr20 | 5164526 | 5164755 | Liver Hepatocytes | 0.05 | 0.91 | intron | CDS2 | U |
| 993 | chr9 | 17436967 | 17437175 | Liver Hepatocytes | 0.08 | 0.93 | intron | CNTLN | U |
| 994 | chr22 | 36694636 | 36695011 | Liver Hepatocytes | 0.07 | 0.92 | intron | MYH9 | U |
| 995 | chr22 | 18244342 | 18244496 | Liver Hepatocytes | 0.1 | 0.94 | intron | BID | U |
| 996 | chr22 | 40389141 | 40389279 | Liver Hepatocytes | 0.05 | 0.88 | Intergenic | FAM83F | U |
| 997 | chr20 | 329660741 | 32966265 | Liver Hepatocytes | 0.11 | 0.94 | intron | ITCH | U |
| 998 | chr6 | 11057852 | 11058182 | Liver Hepatocytes | 0.09 | 0.92 | intron | ELOVL2-AS1 | U |
| 999 | chr14 | 73570848 | 3571295 | Liver Hepatocytes | 0.07 | 0.9 | intron | RBM25 | U |
| 1000 | chr22 | 21124715 | 21124992 | Liver Hepatocytes | 0.09 | 0.91 | intron | PI4KA | U |
| 1001 | chr22 | 41742512 | 41742638 | Liver Hepatocytes | 0.15 | 0.92 | intron | ZC3H78 | U |
| 1002 | chr19 | 191649891 | 19165282 | Liver Hepatocytes | 0.14 | 0.91 | exon, intron | ARMC6, ARMC6 | U |
| 1003 | chr3 | 1972812631 | 197281406 | Liver Hepatocytes | 0.91 | 0.08 | intron | BDH1 | M |
| 1004 | chr8 | 141607110 | 141607587 | Liver Hepatocytes | 0.86 | 0.05 | intron | AGO2 | M |
| 1005 | chr5 | 143206485 | 143206826 | Liver Hepatocytes | 0.84 | 0.04 | intron | HIVEP2 | M |
| 1006 | chr1 | 230560233 | 230560712 | Liver Hepatocytes | 0.83 | 0.07 | intron | PGBD5 | M |
| 1007 | chr4 | 73998063 | 7400062 | Liver Hepatocytes | 0.88 | 0.14 | intron | SORCS2 | M |
| 1008 | chr2 | 233585441 | 23358852 | Liver Hepatocytes | 0.84 | 0.1 | Intergenic | KLHL29 | M |
| 1009 | chr2 | 44058696 | 44058786 | Liver Hepatocytes | 0.81 | 0.09 | intron | ABCG5 | M |
| 1010 | chr16 | 5167435 | 5167789 | Liver Hepatocytes | 0.72 | 0.03 | Intergenic | FAM86A | M |
| 1011 | chr10 | 1038808581 | 103881174 | Liver Hepatocytes | 0.73 | 0.07 | exon, promoter-TSS | LDB1, LDB1 | M |
| 1012 | chr12 | 1032729551 | 103273198 | Liver Hepatocytes | 0.69 | 0.04 | intron | PAH | M |
| 1013 | chr3 | 107084101 | 10708578 | Liver Hepatocytes | 0.81 | 0.18 | Intergenic | LINC00606 | M |
| 1014 | chr12 | 109729450 | 109729728 | Liver Hepatocytes | 0.64 | 0.02 | intron | FOXN4 | M |
| 1015 | chr18 | 550998981 | 55100290 | Liver Hepatocytes | 0.66 | 0.08 | Intergenic | ONECUT2 | M |
| 1016 | chr7 | 1006112973 | 100611720 | Liver Hepatocytes | 0.64 | 0.07 | Intergenic | MUC12 | M |
| 1017 | chr13 | 466274271 | 46627507 | Liver Hepatocytes | 0.62 | 0.06 | promoter-TSS | ZC3H13 | M |
| 1018 | chr17 | 56591006 | 56591496 | Liver Hepatocytes | 0.59 | 0.07 | intron | MTMR4 | M |
| 1019 | chr16 | 87943033 | 87943236 | Liver Hepatocytes | 0.8 | 0.02 | intron | CA5A | M |
| 1020 | chr3 | 55515250 | 55515736 | Liver Hepatocytes | 0.81 | 0.03 | promoter-TSS | WNT5A | M |
| 1021 | chr7 | 878481433 | 87848452 | Liver Hepatocytes | 0.8 | 0.03 | intron | SRI | M |
| 1022 | chr4 | 10020842 | 10020997 | Liver Hepatocytes | 0.76 | 0.04 | intron | SLC2A9 | M |
| 1023 | chr19 | 36389546 | 36389967 | Liver Hepatocytes | 0.71 | 0.02 | exon, intron | NFKBID, NFKBID | M |
| 1024 | chr13 | 113765195 | 113765320 | Liver Hepatocytes | 0.85 | 0.06 | intron | F7 | M |
| 1025 | chr14 | 59656966 | 59657244 | Liver Hepatocytes | 0.8 | 0.08 | intron | DAAM1 | M |
| 1026 | chr19 | 15580657 | 15580958 | Liver Hepatocytes | 0.75 | 0.05 | intron | PGLYRP2 | M |
| 1027 | chr4 | 10020419 | 10020794 | Liver Hepatocytes | 0.58 | 0.01 | exon | SLC2A9 | M |
| 1028 | chr14 | 81997731 | 81998179 | Pancreatic Acinar cells | 0.02 | 0.93 | intron | SEL1L | U |
| 1029 | chr11 | 117183396 | 117183511 | Pancreatic Acinar cells | 0.04 | 0.95 | intron | BACE1 | U |
| 1030 | chr4 | 31797721 | 3180231 | Pancreatic Acinar cells | 0.03 | 0.93 | intron | HTT | U |
| 1031 | chr17 | 71025197 | 71025671 | Pancreatic Acinar cells | 0.05 | 0.95 | intron | SLC39A11 | U |
| 1032 | chr7 | 97858311 | 97858590 | Pancreatic Acinar cells | 0.04 | 0.93 | intron | TECPR1 | U |
| 1033 | chr8 | 37891295 | 37891559 | Pancreatic Acinar cells | 0.06 | 0.95 | intron | EIF4EBP1 | U |
| 1034 | chr1 | 2301939993 | 230194357 | Pancreatic Acinar cells | 0.02 | 0.9 | Intergenic | GALNT2 | U |
| 1035 | chr16 | 630414 | 630878 | Pancreatic Acinar cells | 0.06 | 0.94 | intron | PIGQ | U |
| 1036 | chr7 | 2415447 | 2415667 | Pancreatic Acinar cells | 0.05 | 0.92 | intron | EIF38 | U |
| 1037 | chr7 | 97842283 | 97842419 | Pancreatic Acinar cells | 0.03 | 0.89 | TTS | BHLHA15 | U |
| 1038 | chr6 | 134540439 | 134540669 | Pancreatic Acinar cells | 0.08 | 0.94 | intron | SGK1 | U |
| 1039 | chr12 | 120741847 | 120742015 | Pancreatic Acinar cells | 0.04 | 0.89 | intron | SIRT4 | U |
| 1040 | chr15 | 90610401 | 90610702 | Pancreatic Acinar cells | 0.09 | 0.93 | intron | ZNF710 | U |
| 1041 | chr16 | 25181855 | 25182322 | Pancreatic Acinar cells | 0.11 | 0.95 | intron | LCMT1 | U |
| 1042 | chr7 | 97844610 | 97844822 | Pancreatic Acinar cells | 0.01 | 0.92 | TTS | TECPR1 | U |
| 1043 | chr12 | 1208932181 | 120893558 | Pancreatic Acinar cells | 0.02 | 0.93 | intron | GATC | U |
| 1044 | chr7 | 129910980 | 129911336 | Pancreatic Acinar cells | 0.03 | 0.94 | intron | CPA2 | U |
| 1045 | chr4 | 186605879 | 186605996 | Pancreatic Acinar cells | 0.03 | 0.93 | exon | SORBS2 | U |
| 1046 | chr1 | 22331421 | 22331618 | Pancreatic Acinar cells | 0.02 | 0.92 | intron | CELA3A | U |
| 1047 | chr2 | 182774084 | 182774369 | Pancreatic Acinar cells | 0.03 | 0.93 | intron | SSFA2 | U |
| 1048 | chr1 | 223300363 | 22330359 | Pancreatic Acinar cells | 0.02 | 0.92 | intron | CELA3A | U |
| 1049 | chr16 | 679639051 | 67964349 | Pancreatic Acinar cells | 0.03 | 0.93 | TTS, TTS | CTRL, PSKH1 | U |
| 1050 | chr1 | 15767029 | 15767514 | Pancreatic Acinar cells | 0.03 | 0.93 | intron | CTRC | U |
| 1051 | chr9 | 135940264 | 135940641 | Pancreatic Acinar cells | 0.03 | 0.92 | exon | CEL | U |
| 1052 | chr19 | 39692865 | 39692936 | Pancreatic Acinar cells | 0.03 | 0.91 | TTS | NCCRP1 | U |
| 1053 | chr12 | 8370881 | 8371049 | Pancreatic Acinar cells | 0.03 | 0.91 | Intergenic | FAM90A1 | U |
| 1054 | chr17 | 76178604 | 76178888 | Pancreatic Acinar cells | 0.05 | 0.93 | intron | TK1 | U |
| 1055 | chr6 | 170577808 | 170578184 | Pancreatic Acinar cells | 0.03 | 0.91 | Intergenic | LOC154449 | U |
| 1056 | chr18 | 77448085 | 77448179 | Pancreatic Acinar cells | 0.02 | 0.89 | intron | CTDP1 | U |
| 1057 | chr12 | 8371595 | 8371733 | Pancreatic Acinar cells | 0.02 | 0.89 | Intergenic | FAM9041 | U |
| 1058 | chr7 | 129614765 | 129614974 | Pancreatic Acinar cells | 0.03 | 0.9 | Intergenic | UBE2H | U |
| 1059 | chr11 | 65148746 | 65148988 | Pancreatic Acinar cells | 0.04 | 0.91 | intron | SLC25A45 | U |
| 1060 | chr1 | 42505787 | 42506136 | Pancreatic Acinar cells | 0.03 | 0.9 | Intergenic | HIVEP3 | U |
| 1061 | chr2 | 241266440 | 241266587 | Pancreatic Acinar cells | 0.04 | 0.9 | Intergenic | GPC1 | U |
| 1062 | chr13 | 113295841 | 113296010 | Pancreatic Acinar cells | 0.03 | 0.89 | Intergenic | C13orf35 | U |
| 1063 | chr9 | 135948843 | 135949021 | Pancreatic Acinar cells | 0.02 | 0.88 | Intergenic | CELP | U |
| 1064 | chr10 | 134224276 | 134224514 | Pancreatic Acinar cells | 0.06 | 0.92 | intron | PWWP2B | U |
| 1065 | chr10 | 118309614 | 118309859 | Pancreatic Acinar cells | 0.03 | 0.89 | intron | PNLIP | U |
| 1066 | chr2 | 135198029 | 135198417 | Pancreatic Acinar cells | 0.08 | 0.94 | intron | MGAT5 | U |
| 1067 | chr2 | 242847380 | 242847794 | Pancreatic Acinar cells | 0.06 | 0.92 | Intergenic | CXXC11 | U |
| 1068 | chr1 | 22331987 | 22332079 | Pancreatic Acinar cells | 0.03 | 0.88 | exon | CELA3A | U |
| 1069 | chr5 | 172408850 | 172409140 | Pancreatic Acinar cells | 0.02 | 0.87 | Intergenic | ATP6V0E1 | U |
| 1070 | chr6 | 35766021 | 35766339 | Pancreatic Acinar cells | 0.01 | 0.86 | Intergenic | CLPS | U |
| 1071 | chr12 | 120764208 | 120764549 | Pancreatic Acinar cells | 0.02 | 0.87 | intron | PLA2G1B | U |
| 1072 | chr16 | 4763799 | 4764173 | Pancreatic Acinar cells | 0.04 | 0.89 | intron | ANKS3 | U |
| 1073 | chr11 | 76076294 | 76076715 | Pancreatic Acinar cells | 0.08 | 0.93 | intron | PRKRIR | U |
| 1074 | chr1 | 26133259 | 26133406 | Pancreatic Acinar cells | 0.1 | 0.94 | intron | SEPN1 | U |
| 1075 | chr1 | 42243440 | 42243601 | Pancreatic Acinar cells | 0.09 | 0.93 | intron | HIVEP3 | U |
| 1076 | chr17 | 3808720 | 3808913 | Pancreatic Acinar cells | 0.04 | 0.88 | intron | P2RX1 | U |
| 1077 | chr2 | 97267907 | 97268130 | Pancreatic Acinar cells | 0.07 | 0.91 | exon | KANSL3 | U |
| 1078 | chr17 | 79814901 | 79815185 | Pancreatic Acinar cells | 0.08 | 0.92 | intron | P4HB | U |
| 1079 | chr12 | 120761832 | 120762191 | Pancreatic Acinar cells | 0.03 | 0.87 | intron | PLA2G1B | U |
| 1080 | chr2 | 46144305 | 46144713 | Pancreatic Acinar cells | 0.1. | 0.94 | intron | PRKCE | U |
| 1081 | chr1 | 230290496 | 230290954 | Pancreatic Acinar cells | 0.07 | 0.91 | intron | GALNT2 | U |
| 1082 | chr10 | 15273069 | 15273528 | Pancreatic Acinar cells | 0.07 | 0.91 | intron | FAM171A1 | U |
| 1083 | chr9 | 140415142 | 140415254 | Pancreatic Acinar cells | 0.07 | 0.9 | intron | PNPLA7 | U |
| 1084 | chr2 | 241638157 | 241638274 | Pancreatic Acinar cells | 0.06 | 0.89 | TTS | AQP12A | U |
| 1085 | chr1 | 185152942 | 185153271 | Pancreatic Acinar cells | 0.06 | 0.89 | intron | SWT1 | U |
| 1086 | chr9 | 134199248 | 134199675 | Pancreatic Acinar cells | 0.04 | 0.87 | Intergenic | PPAPDC3 | U |
| 1087 | chr11 | 62539791 | 62539982 | Pancreatic Acinar cells | 0.04 | 0.86 | intron | TAF6L | U |
| 1088 | chr4 | 1094258 | 1094499 | Pancreatic Acinar cells | 0.1 | 0.92 | intron | RNF212 | U |
| 1089 | chr12 | 126676605 | 126676921 | Pancreatic Acinar cells | 0.02 | 0.84 | Intergenic | LOC400084 | U |
| 1090 | chr16 | 19626697 | 19627017 | Pancreatic Acinar cells | 0.1 | 0.92 | intron | C16orf62 | U |
| 1091 | chr2 | 241632818 | 241633153 | Pancreatic Acinar cells | 0.06 | 0.88 | intron | AQP12A | U |
| 1092 | chr16 | 25229769 | 25230149 | Pancreatic Acinar cells | 0.03 | 0.85 | intron | AQP8 | U |
| 1093 | chr3 | 171789927 | 171790338 | Pancreatic Acinar cells | 0.09 | 0.91 | intron | FNDC3B | U |
| 1094 | chr12 | 120892262 | 120892369 | Pancreatic Acinar cells | 0.11 | 0.92 | intron | GATC | U |
| 1095 | chr14 | 101007293 | 101007792 | Pancreatic Acinar cells | 0.08 | 0.89 | intron | BEGAIN | U |
| 1096 | chr9 | 125705958 | 125706227 | Pancreatic Acinar cells | 0.12 | 0.92 | intron | RABGAP1 | U |
| 1097 | chr3 | 148567923 | 148568291 | Pancreatic Acinar cells | 0.05 | 0.85 | intron | CPB1 | U |
| 1098 | chr11 | 77283866 | 77284088 | Pancreatic Acinar cells | 0.14 | 0.93 | Intergenic | AQP11 | U |
| 1099 | chr19 | 10950193 | 10950550 | Pancreatic Acinar cells | 0.12 | 0.91 | Intergenic | TMED1 | U |
| 1100 | chr14 | 101006856 | 101007144 | Pancreatic Acinar cells | 0.11 | 0.89 | intron | BEGAIN | U |
| 1101 | chr19 | 4936033 | 4936335 | Pancreatic Acinar cells | 0.13 | 0.91 | intron | UHRF1 | U |
| 1102 | chr22 | 43275974 | 43276301 | Pancreatic Acinar cells | 0.13 | 0.91 | intron | PACSIN2 | U |
| 1103 | chr10 | 80806029 | 80806412 | Pancreatic Acinar cells | 0.14 | 0.92 | intron | ZMIZ1-AS1 | U |
| 1104 | chr16 | 87130700 | 87130875 | Pancreatic Acinar cells | 0.15 | 0.92 | Intergenic | C16orf95 | U |
| 1105 | chr5 | 177020076 | 177020544 | Pancreatic Acinar cells | 0.13 | 0.87 | intron | TMED9 | U |
| 1106 | chr3 | 148576081 | 148576341 | Pancreatic Acinar cells | 0.16 | 0.92 | intron | CPB1 | U |
| 1107 | chr12 | 120893581 | 120893926 | Pancreatic Acinar cells | 0.18 | 0.94 | intron | GATC | U |
| 1108 | chr1 | 28846374 | 28846793 | Pancreatic Acinar cells | 0.19 | 0.94 | intron | RCC1 | U |
| 1109 | chr17 | 71264563 | 71264808 | Pancreatic Acinar cells | 0.2 | 0.92 | Intergenic | CPSF4L | U |
| 1110 | chr16 | 88012199 | 88012511 | Pancreatic Acinar cells | 0.23 | 0.93 | intron | BANP | U |
| 1111 | chr15 | 90771745 | 90771965 | Pancreatic Acinar cells | 0.23 | 0.92 | exon | SEMA4B | U |
| 1112 | chr11 | 5704702 | 5704863 | Pancreatic Acinar cells | 0.3 | 0.95 | intron | TRIM5 | U |
| 1113 | chr7 | 116225201 | 116225369 | Pancreatic Acinar cells | 0.03 | 0.94 | Intergenic | CAV1 | U |
| 1114 | chr8 | 49668664 | 49668854 | Pancreatic Acinar cells | 0.02 | 0.92 | Intergenic | EFCAB1 | U |
| 1115 | chr7 | 100320415 | 100320683 | Pancreatic Acinar cells | 0.03 | 0.89 | intron | EPO | U |
| 1116 | chr10 | 120426551 | 120426615 | Pancreatic Acinar cells | 0.06 | 0.89 | Intergenic | PRLHR | U |
| 1117 | chr13 | 1011848243 | 101184990 | Pancreatic Acinar cells | 0.03 | 0.96 | exon | GGACT | U |
| 1118 | chr22 | 26958816 | 26958892 | Pancreatic Acinar cells | 0.02 | 0.94 | intron | TPST2 | U |
| 1119 | chr17 | 80395185 | 80395450 | Pancreatic Acinar cells | 0.02 | 0.93 | exon, intron | HEXDC | U |
| 1120 | chr3 | 148555831 | 148556261 | Pancreatic Acinar cells | 0.03 | 0.93 | intron | CPB1 | U |
| 1121 | chr16 | 75263335 | 75263738 | Pancreatic Acinar cells | 0.06 | 0.95 | exon | BCAR1 | U |
| 1122 | chr16 | 25228659 | 25228751 | Pancreatic Acinar cells | 0.02 | 0.9 | exon | AQP8 | U |
| 1123 | chr9 | 131857131 | 131857496 | Pancreatic Acinar cells | 0.04 | 0.91 | TTS, intron | CRAT | U |
| 1124 | chr11 | 868452 | 868668 | Pancreatic Acinar cells | 0.04 | 0.9 | TTS, exon | CHID1, TSPAN4 | U |
| 1125 | chr22 | 22041093 | 22041568 | Pancreatic Acinar cells | 0.12 | 0.95 | exon | PPIL2 | U |
| 1126 | chr2 | 86282085 | 86282481 | Pancreatic Acinar cells | 0.13 | 0.95 | intron | POLR1A | U |
| 1127 | chr20 | 47601901 | 47602272 | Pancreatic Acinar cells | 0.17 | 0.95 | intron | ARFGEF2 | U |
| 1128 | chr20 | 43938811 | 43939225 | Pancreatic Acinar cells | 0.04 | 0.94 | intron | RBPJL | U |
| 1129 | chr19 | 39691013 | 39691058 | Pancreatic Acinar cells | 0.02 | 0.91 | exon | NCCRP1 | U |
| 1130 | chr2 | 241631375 | 241631499 | Pancreatic Acinar cells | 0.03 | 0.92 | exon | AQP12A | U |
| 1131 | chr15 | 42211291 | 42211586 | Pancreatic Acinar cells | 0.05 | 0.94 | exon, intron | EHD4, EHD4 | U |
| 1132 | chr9 | 1403517361 | 140351908 | Pancreatic Acinar cells | 0.05 | 0.93 | intron | NSMF | U |
| 1133 | chr20 | 43938184 | 43938419 | Pancreatic Acinar cells | 0.03 | 0.91 | intron | RBPJL | U |
| 1134 | chr12 | 51749472 | 51749712 | Pancreatic Acinar cells | 0.06 | 0.92 | exon | GALNT6 | U |
| 1135 | chr22 | 24179883 | 24180181 | Pancreatic Acinar cells | 0.05 | 0.91 | exon, intron | DERL3, DERL3 | U |
| 1136 | chr11 | 65147920 | 65147974 | Pancreatic Acinar cells | 0.05 | 0.9 | intron | SLC25A45 | U |
| 1137 | chr22 | 44671168 | 44671297 | Pancreatic Acinar cells | 0.06 | 0.91 | intron | KIAA1644 | U |
| 1138 | chr7 | 129501469 | 129501600 | Pancreatic Acinar cells | 0.09 | 0.94 | intron | UBE2H | U |
| 1139 | chr9 | 35295715 | 35295918 | Pancreatic Acinar cells | 0.07 | 0.92 | intron | UNC13B | U |
| 1140 | chr16 | 20335338 | 20335551 | Pancreatic Acinar cells | 0.02 | 0.87 | exon | GP2 | U |
| 1141 | chr20 | 43948863 | 43949159 | Pancreatic Acinar cells | 0.04 | 0.89 | Intergenic | MATN4 | U |
| 1142 | chr19 | 500599193 | 50060293 | Pancreatic Acinar cells | 0.03 | 0.88 | intron, exon, intron | NOSIP, NOSIP, NOSIP | U |
| 1143 | chr16 | 75262836 | 75263298 | Pancreatic Acinar cells | 0.05 | 0.9 | TTS, exon | BCAR1, BCAR1 | U |
| 1144 | chr16 | 85351181 | 85351440 | Pancreatic Acinar cells | 0.09 | 0.93 | Intergenic | MIR5093 | U |
| 1145 | chr19 | 6026434 | 6026791 | Pancreatic Acinar cells | 0.09 | 0.93 | intron | RFX2 | U |
| 1146 | chr19 | 39691205 | 39691581 | Pancreatic Acinar cells | 0.02 | 0.86 | exon | NCCRP1 | U |
| 1147 | chr22 | 22041111 | 22041569 | Pancreatic Acinar cells | 0.11 | 0.95 | exon | PPIL2 | U |
| 1148 | chr7 | 97602665 | 97602853 | Pancreatic Acinar cells | 0.05 | 0.88 | Intergenic | MGC72080 | U |
| 1149 | chr19 | 7535541 | 7535800 | Pancreatic Acinar cells | 0.13 | 0.93 | intron, exon | ARHGEF18, ARHGEF1 [symbol] | U |
| 1150 | chr19 | 51589287 | 51589641 | Pancreatic Acinar cells | 0.06 | 0.89 | Intergenic | KLK14 | U |
| 1151 | chr20 | 30904803 | 30905203 | Pancreatic Acinar cells | 0.16 | 0.96 | intron | KIF38 | U |
| 1152 | chr19 | 19576160 | 19576557 | Pancreatic Acinar cells | 0.13 | 0.92 | exon | GATAD2A | U |
| 1153 | chr6 | 7310866 | 7311139 | Pancreatic Acinar cells | 0.14 | 0.92 | intron | SSR1 | U |
| 1154 | chr16 | 28914746 | 28915197 | Pancreatic Acinar cells | 0.09 | 0.87 | TTS | RABEP2 | U |
| 1155 | chr3 | 1840157381 | 184016106 | Pancreatic Acinar cells | 0.19 | 0.91 | Intergenic | PSMD2 | U |
| 1156 | chr1 | 22352744 | 22352894 | Pancreatic Acinar cells | 0.81 | 0.06 | intron | LINC00339 | M |
| 1157 | chr4 | 3486324 | 3486623 | Pancreatic Acinar cells | 0.81 | 0.11 | intron | DOK7 | M |
| 1158 | chr3 | 1333930453 | 133393165 | Pancreatic Acinar cells | 0.79 | 0.14 | Intergenic | TOPBP1 | M |
| 1159 | chr5 | 139283262 | 139283410 | Pancreatic Acinar cells | 0.71 | 0.07 | intron | NRG2 | M |
| 1160 | chr7 | 107786526 | 107786982 | Pancreatic Acinar cells | 0.58 | 0.05 | Intergenic | LAMB4 | M |
| 1161 | chr18 | 20140157 | 20140339 | Pancreatic Acinar cells | 0.56 | 0.05 | Intergenic | CTAGE1 | M |
| 1162 | chr12 | 25055441 | 25055771 | Pancreatic Acinar cells | 0.9 | 0.08 | promoter-TSS | BCAT1 | M |
| 1163 | chr13 | 28491265 | 28491662 | Pancreatic Acinar cells | 0.88 | 0.06 | Intergenic | PDX1 | M |
| 1164 | chr7 | 139168804 | 139169080 | Pancreatic Acinar cells | 0.94 | 0.13 | promoter-TSS | KLRG2 | M |
| 1165 | chr7 | 157361420 | 157361854 | Pancreatic Acinar cells | 0.84 | 0.03 | exon | PTPRN2 | M |
| 1166 | chr10 | 52178282 | 52178405 | Pancreatic Acinar cells | 0.85 | 0.05 | intron | SGMS1 | M |
| 1167 | chr12 | 65218052 | 65218171 | Pancreatic Acinar cells | 0.82 | 0.05 | promoter-TSS | TBC1D30 | M |
| 1168 | chr15 | 53089906 | 53090193 | Pancreatic Acinar cells | 0.85 | 0.08 | Intergenic | ONECUT1 | M |
| 1169 | chr14 | 74208161 | 74208539 | Pancreatic Acinar cells | 0.79 | 0.04 | intron | ELMSAN1 | M |
| 1170 | chr12 | 1177979153 | 117798353 | Pancreatic Acinar cells | 0.78 | 0.06 | intron | NOS1 | M |
| 1171 | chr4 | 1165716 | 1165975 | Pancreatic Acinar cells | 0.78 | 0.07 | intron | SPON2 | M |
| 1172 | chr7 | 127176750 | 127177145 | Pancreatic Acinar cells | 0.79 | 0.09 | Intergenic | GCC1 | M |
| 1173 | chr17 | 61553811 | 61554180 | Pancreatic Acinar cells | 0.72 | 0.04 | promoter-TSS | ACE | M |
| 1174 | chr11 | 123946941 | 123947372 | Pancreatic Acinar cells | 0.73 | 0.02 | Intergenic | OR10G7 | M |
| 1175 | chr11 | 1410140 | 1410191 | Pancreatic Acinar cells | 0.86 | 0.19 | promoter-TSS | BRSK2 | M |
| 1176 | chr8 | 11536821 | 11537026 | Pancreatic Acinar cells | 0.77 | 0.1 | Intergenic | GATA4 | M |
| 1177 | chr20 | 43945377 | 43945435 | Pancreatic Acinar cells | 0.85 | 0.2 | exon | RBPJL | M |
| 1178 | chr8 | 11537046 | 11537196 | Pancreatic Acinar cells | 0.71 | 0.08 | Intergenic | GATA4 | M |
| 1179 | chr22 | 38221073 | 38221383 | Pancreatic Acinar cells | 0.66 | 0.04 | exon | GALR3 | M |
| 1180 | chr16 | 67208453 | 67208631 | Pancreatic Acinar cells | 0.61 | 0.04 | exon | NOL3 | M |
| 1181 | chr9 | 136894752 | 136894939 | Pancreatic Alpha cells | 0.01 | 0.93 | TTS | BRD3 | U |
| 1182 | chr12 | 123058908 | 123059162 | Pancreatic Alpha cells | 0.03 | 0.94 | intron | KNTC1 | U |
| 1183 | chr4 | 3305594 | 3305918 | Pancreatic Alpha cells | 0.02 | 0.89 | Intergenic | RGS12 | U |
| 1184 | chr11 | 8258933 | 8259173 | Pancreatic Alpha cells | 0.05 | 0.91 | intron | LMO1 | U |
| 1185 | chr9 | 122767613 | 122767941 | Pancreatic Alpha cells | 0.02 | 0.88 | Intergenic | MIR147A | U |
| 1186 | chr18 | 5399141 | 5399588 | Pancreatic Alpha cells | 0.03 | 0.88 | intron | EPB41L3 | U |
| 1187 | chr5 | 57310246 | 57310709 | Pancreatic Alpha cells | 0.06 | 0.89 | Intergenic | PLK2 | U |
| 1188 | chr2 | 163006618 | 163007095 | Pancreatic Alpha cells | 0.04 | 0.87 | intron | GCG | U |
| 1189 | chr19 | 38695130 | 38695460 | Pancreatic Alpha cells | 0.11 | 0.92 | intron | SIPA1L3 | U |
| 1190 | chr14 | 93655063 | 93655455 | Pancreatic Alpha cells | 0.1 | 0.92 | Intergenic | TMEM251 | U |
| 1191 | chr4 | 128912121 | 128912567 | Pancreatic Alpha cells | 0.12 | 0.94 | intron | C4orf29 | U |
| 1192 | chr6 | 33672216 | 33672385 | Pancreatic Alpha cells | 0.06 | 0.86 | intron | MNF1 | U |
| 1193 | chr6 | 157907038 | 157907401 | Pancreatic Alpha cells | 0.12 | 0.92 | intron | ZDHHC14 | U |
| 1194 | chr11 | 33666715 | 33667028 | Pancreatic Alpha cells | 0.11 | 0.9 | intron | KIAA1549L | U |
| 1195 | chr13 | 24244799 | 24245022 | Pancreatic Alpha cells | 0.15 | 0.9 | intron | TNFRSF19 | U |
| 1196 | chr6 | 159183176 | 159183405 | Pancreatic Alpha cells | 0.13 | 0.88 | intron | SYTL3 | U |
| 1197 | chr7 | 1152687 | 1152806 | Pancreatic Alpha cells | 0.21 | 0.94 | intron | C7orf50 | U |
| 1198 | chr8 | 140863882 | 140864141 | Pancreatic Alpha cells | 0.28 | 0.95 | intron | TRAPPC9 | U |
| 1199 | chr17 | 65148228 | 65148582 | Pancreatic Alpha cells | 0.04 | 0.93 | intron | HELZ | U |
| 1200 | chr8 | 19270044 | 19270130 | Pancreatic Alpha cells | 0.03 | 0.91 | intron | CSGALNACT1 | U |
| 1201 | chr18 | 5398260 | 5398658 | Pancreatic Alpha cells | 0.02 | 0.9 | intron | EPB41L3 | U |
| 1202 | chr7 | 41528144 | 41528284 | Pancreatic Alpha cells | 0.03 | 0.89 | Intergenic | INHBA-AS1 | U |
| 1203 | chr18 | 5398664 | 5399022 | Pancreatic Alpha cells | 0.08 | 0.94 | intron | EPB41L3 | U |
| 1204 | chr2 | 236588582 | 236588647 | Pancreatic Alpha cells | 0.01 | 0.86 | intron | AGAP1 | U |
| 1205 | chr14 | 93490477 | 93490558 | Pancreatic Alpha cells | 0.08 | 0.93 | intron | ITPK1 | U |
| 1206 | chr21 | 37580197 | 37580399 | Pancreatic Alpha cells | 0.04 | 0.89 | intron | DOPEY2 | U |
| 1207 | chr7 | 3964943 | 3965209 | Pancreatic Alpha cells | 0.02 | 0.87 | intron | SDK1 | U |
| 1208 | chr2 | 191749899 | 191750262 | Pancreatic Alpha cells | 0.03 | 0.88 | intron | GLS | U |
| 1209 | chr10 | 127219667 | 127219682 | Pancreatic Alpha cells | 0.06 | 0.9 | Intergenic | LOC100169752 | U |
| 1210 | chr3 | 32381347 | 32381434 | Pancreatic Alpha cells | 0.05 | 0.89 | intron | CMTM8 | U |
| 1211 | chr10 | 1021209 | 1021351 | Pancreatic Alpha cells | 0.05 | 0.88 | Intergenic | GTPBP4 | U |
| 1212 | chr11 | 67871149 | 67871329 | Pancreatic Alpha cells | 0.08 | 0.91 | intron | CHKA | U |
| 1213 | chr4 | 141065012 | 141065244 | Pancreatic Alpha cells | 0.11 | 0.94 | intron | MAML3 | U |
| 1214 | chr6 | 170175943 | 170176162 | Pancreatic Alpha cells | 0.09 | 0.91 | exon | C6orf70 | U |
| 1215 | chr3 | 170059223 | 170059480 | Pancreatic Alpha cells | 0.04 | 0.86 | Intergenic | SKIL | U |
| 1216 | chr13 | 60435635 | 60435892 | Pancreatic Alpha cells | 0.06 | 0.88 | intron | DIAPH3 | U |
| 1217 | chr17 | 79222427 | 79222822 | Pancreatic Alpha cells | 0.07 | 0.88 | intron | SLC38A10 | U |
| 1218 | chr1 | 39952315 | 39952539 | Pancreatic Alpha cells | 0.11 | 0.9 | exon | MACF1 | U |
| 1219 | chr19 | 53049737 | 53049983 | Pancreatic Alpha cells | 0.13 | 0.92 | intron | ZNF808 | U |
| 1220 | chr2 | 49161981 | 49162312 | Pancreatic Alpha cells | 0.07 | 0.86 | Intergenic | LHCGR | U |
| 1221 | chr12 | 107866102 | 107866486 | Pancreatic Alpha cells | 0.1 | 0.89 | intron | BTBD11 | U |
| 1222 | chr4 | 3004786 | 3005003 | Pancreatic Alpha cells | 0.14 | 0.92 | intron | GRK4 | U |
| 1223 | chr6 | 153441728 | 153442021 | Pancreatic Alpha cells | 0.11 | 0.89 | intron | RGS17 | U |
| 1224 | chr8 | 2803474 | 2803845 | Pancreatic Alpha cells | 0.07 | 0.85 | intron | CSMD1 | U |
| 1225 | chr7 | 79840316 | 79840385 | Pancreatic Alpha cells | 0.13 | 0.9 | exon | GNAI1 | U |
| 1226 | chr3 | 177085908 | 177086174 | Pancreatic Alpha cells | 0.12 | 0.89 | Intergenic | LINC00578 | U |
| 1227 | chr15 | 57665395 | 57665796 | Pancreatic Alpha cells | 0.07 | 0.84 | Intergenic | CGNL1 | U |
| 1228 | chr2 | 163062923 | 163063357 | Pancreatic Alpha cells | 0.14 | 0.91 | intron | FAP | U |
| 1229 | chr10 | 75601807 | 75601893 | Pancreatic Alpha cells | 0.12 | 0.88 | intron | CAMK2G | U |
| 1230 | chr18 | 53212032 | 53212209 | Pancreatic Alpha cells | 0.14 | 0.9 | intron | TCF4 | U |
| 1231 | chr13 | 114111683 | 114111908 | Pancreatic Alpha cells | 0.12 | 0.88 | exon | DCUN1D2 | U |
| 1232 | chr21 | 17026618 | 17026965 | Pancreatic Alpha cells | 0.14 | 0.9 | Intergenic | USP25 | U |
| 1233 | chr8 | 1413986233 | 141398837 | Pancreatic Alpha cells | 0.14 | 0.89 | Intergenic | MIR4465 | U |
| 1234 | chr11 | 15662272 | 15662565 | Pancreatic Alpha cells | 0.07 | 0.82 | Intergenic | INSC | U |
| 1235 | chr7 | 50666848 | 50666929 | Pancreatic Alpha cells | 0.18 | 0.92 | intron | GRB10 | U |
| 1236 | chr12 | 107859460 | 107859678 | Pancreatic Alpha cells | 0.1 | 0.84 | intron | BTBD11 | U |
| 1237 | chr6 | 136920291 | 136920652 | Pancreatic Alpha cells | 0.13 | 0.87 | intron | MAP3K5 | U |
| 1238 | chr17 | 3745633 | 3745853 | Pancreatic Alpha cells | 0.22 | 0.95 | intron | C17orf85 | U |
| 1239 | chr2 | 101892145 | 101892366 | Pancreatic Alpha cells | 0.22 | 0.95 | exon | RNF149 | U |
| 1240 | chr1 | 246828709 | 246829097 | Pancreatic Alpha cells | 0.18 | 0.91 | intron | CNST | U |
| 1241 | chr8 | 93584045 | 93584109 | Pancreatic Alpha cells | 0.18 | 0.9 | Intergenic | FLJ46284 | U |
| 1242 | chr13 | 96827497 | 96827722 | Pancreatic Alpha cells | 0.19 | 0.91 | intron | HS6ST3 | U |
| 1243 | chr2 | 36985565 | 36985846 | Pancreatic Alpha cells | 0.12 | 0.84 | intron | VIT | U |
| 1244 | chr8 | 96051029 | 96051338 | Pancreatic Alpha cells | 0.21 | 0.93 | intron | NDUFAF6 | U |
| 1245 | chr5 | 164754286 | 164754657 | Pancreatic Alpha cells | 0.15 | 0.87 | Intergenic | MAT2B | U |
| 1246 | chrX | 56820581 | 56820750 | Pancreatic Alpha cells | 0.16 | 0.87 | intron | LOC550643 | U |
| 1247 | chr3 | 79999196 | 79999429 | Pancreatic Alpha cells | 0.12 | 0.83 | Intergenic | ROBO1 | U |
| 1248 | chr15 | 36621859 | 36622108 | Pancreatic Alpha cells | 0.15 | 0.86 | Intergenic | C15orf41 | U |
| 1249 | chr15 | 91509125 | 91509503 | Pancreatic Alpha cells | 0.2 | 0.91 | TTS, promoter-TSS | PRC1, LOC100507118 | U |
| 1250 | chr1 | 61411507 | 51411976 | Pancreatic Alpha cells | 0.17 | 0.88 | Intergenic | NFIA | U |
| 1251 | chr11 | 61368781 | 61368942 | Pancreatic Alpha cells | 0.15 | 0.85 | Intergenic | RPLP0P2 | U |
| 1252 | chr3 | 31857960 | 31858297 | Pancreatic Alpha cells | 0.17 | 0.87 | intron | OSBPL10 | U |
| 1253 | chr7 | 153394633 | 153394986 | Pancreatic Alpha cells | 0.15 | 0.84 | Intergenic | DPP6 | U |
| 1254 | chr21 | 45380092 | 45380197 | Pancreatic Alpha cells | 0.25 | 0.93 | intron | AGPAT3 | U |
| 1255 | chr11 | 33576001 | 33576108 | Pancreatic Alpha cells | 0.23 | 0.91 | intron | KIAA1549L | U |
| 1256 | chr18 | 45667935 | 45668049 | Pancreatic Alpha cells | 0.23 | 0.91 | Intergenic | ZBTB7C | U |
| 1257 | chr10 | 1000868481 | 100087183 | Pancreatic Alpha cells | 0.14 | 0.82 | Intergenic | LOXL4 | U |
| 1258 | chr1 | 201671557 | 201671897 | Pancreatic Alpha cells | 0.2 | 0.88 | intron | NAV1 | U |
| 1259 | chr10 | 75598299 | 75598697 | Pancreatic Alpha cells | 0.23 | 0.88 | intron | CAMK2G | U |
| 1260 | chr10 | 99427111 | 99427259 | Pancreatic Alpha cells | 0.25 | 0.92 | intron | PI4K2A | U |
| 1261 | chr7 | 55250136 | 55250413 | Pancreatic Alpha cells | 0.23 | 0.9 | intron | EGFR-AS1 | U |
| 1262 | chr10 | 421964 | 422153 | Pancreatic Alpha cells | 0.23 | 0.89 | intron | DIP2C | U |
| 1263 | chr6 | 148307348 | 148307563 | Pancreatic Alpha cells | 0.22 | 0.88 | Intergenic | SASH1 | U |
| 1264 | chr11 | 6835841 | 68358748 | Pancreatic Alpha cells | 0.23 | 0.89 | intron | PPP6R3 | U |
| 1265 | chr17 | 945834 | 946237 | Pancreatic Alpha cells | 0.26 | 0.92 | intron | ABR | U |
| 1266 | chr3 | 100536916 | 100537151 | Pancreatic Alpha cells | 0.24 | 0.89 | intron | ABI3BP | U |
| 1267 | chr9 | 140718792 | 140719046 | Pancreatic Alpha cells | 0.23 | 0.88 | intron | EHMT1 | U |
| 1268 | chr16 | 66577370 | 66577702 | Pancreatic Alpha cells | 0.2 | 0.85 | intron | TK2 | U |
| 1269 | chr4 | 40176314 | 40176379 | Pancreatic Alpha cells | 0.22 | 0.86 | Intergenic | RHOH | U |
| 1270 | chr10 | 27024210 | 27024361 | Pancreatic Alpha cells | 0.27 | 0.9 | exon, intron | PDSS1, PDSS1 | U |
| 1271 | chr3 | 8687845 | 8688230 | Pancreatic Alpha cells | 0.28 | 0.9 | intron | SSUH2 | U |
| 1272 | chr5 | 76171518 | 76171951 | Pancreatic Alpha cells | 0.19 | 0.81 | intron | S100Z | U |
| 1273 | chr6 | 159100930 | 159101081 | Pancreatic Alpha cells | 0.22 | 0.83 | intron | SYTL3 | U |
| 1274 | chr6 | 168668214 | 168668548 | Pancreatic Alpha cells | 0.27 | 0.88 | Intergenic | DACT2 | U |
| 1275 | chr12 | 109959317 | 109959565 | Pancreatic Alpha cells | 0.31 | 0.91 | exon, intron | UBE3B, UBE38 | U |
| 1276 | chr17 | 28538035 | 28538194 | Pancreatic Alpha cells | 0.3 | 0.89 | intron | SLC6A4 | U |
| 1277 | chr17 | 5756865 | 5757052 | Pancreatic Alpha cells | 0.25 | 0.83 | intron | LOC339166 | U |
| 1278 | chr1 | 94595612 | 94596059 | Pancreatic Alpha cells | 0.3 | 0.88 | Intergenic | ABCA4 | U |
| 1279 | chr2 | 195176961 | 195177002 | Pancreatic Alpha cells | 0.29 | 0.86 | Intergenic | SLC39A10 | U |
| 1280 | chr1 | 22439457 | 22439736 | Pancreatic Alpha cells | 0.36 | 0.92 | Intergenic | WNT4 | U |
| 1281 | chr11 | 33534024 | 33534354 | Pancreatic Alpha cells | 0.36 | 0.9 | Intergenic | KIAA1549L | U |
| 1282 | chr8 | 9448608 | 9449010 | Pancreatic Alpha cells | 0.31 | 0.85 | intron | TNKS | U |
| 1283 | chr18 | 74659093 | 74659517 | Pancreatic Alpha cells | 0.17 | 0.94 | intron | ZNF236 | U |
| 1284 | chr16 | 3795172 | 3795662 | Pancreatic Alpha cells | 0.32 | 0.93 | exon, intron | CREBBP | U |
| 1285 | chr10 | 128092762 | 128092925 | Pancreatic Alpha cells | 0.09 | 0.86 | Intergenic | ADAM12 | U |
| 1286 | chr3 | 59793504 | 59793615 | Pancreatic Alpha cells | 0.25 | 0.94 | intron | FHIT | U |
| 1287 | chr18 | 74541752 | 74541984 | Pancreatic Alpha cells | 0.32 | 0.95 | intron | ZNF236 | U |
| 1288 | chr22 | 41269489 | 41269560 | Pancreatic Alpha cells | 0.01 | 0.94 | intron | XPNPEP3 | U |
| 1289 | chr14 | 34144321 | 34144555 | Pancreatic Alpha cells | 0.15 | 0.93 | intron | NPAS3 | U |
| 1290 | chr20 | 31950133 | 31950344 | Pancreatic Alpha cells | 0.1 | 0.86 | intron | CDK5RAP1 | U |
| 1291 | chr13 | 113173833 | 113174184 | Pancreatic Alpha cells | 0.28 | 0.95 | intron | TUBGCP3 | U |
| 1292 | chr3 | 12940907 | 12941211 | Pancreatic Alpha cells | 0.35 | 0.93 | exon | IQSEC1 | U |
| 1293 | chr16 | 84553123 | 84553184 | Pancreatic Alpha cells | 0.06 | 0.91 | Intergenic | KIAA1609 | U |
| 1294 | chr20 | 24570628 | 24570737 | Pancreatic Alpha cells | 0.01 | 0.84 | intron | SYNDIG1 | U |
| 1295 | chr16 | 714186 | 714422 | Pancreatic Alpha cells | 0.16 | 0.93 | intron | WDR90 | U |
| 1296 | chr5 | 35231999 | 35232165 | Pancreatic Alpha cells | 0.13 | 0.88 | Intergenic | PRLR | U |
| 1297 | chr20 | 23637374 | 23637541 | Pancreatic Alpha cells | 0.15 | 0.87 | Intergenic | CST3 | U |
| 1298 | chr16 | 713945 | 714183 | Pancreatic Alpha cells | 0.08 | 0.79 | intron | WDR90 | U |
| 1299 | chr22 | 36375406 | 36375671 | Pancreatic Alpha cells | 0.17 | 0.88 | intron | R8FOX2 | U |
| 1300 | chr17 | 2539854 | 2540015 | Pancreatic Alpha cells | 0.22 | 0.9 | intron | PAFAH181 | U |
| 1301 | chr22 | 29678381 | 29678791 | Pancreatic Alpha cells | 0.23 | 0.89 | intron | EWSR1 | U |
| 1302 | chr22 | 38489645 | 38489992 | Pancreatic Alpha cells | 0.25 | 0.9 | intron | BAIAP2L2 | U |
| 1303 | chr22 | 303696493 | 30369746 | Pancreatic Alpha cells | 0.29 | 0.93 | intron | MTMR3 | U |
| 1304 | chr6 | 168709994 | 168710230 | Pancreatic Alpha cells | 0.29 | 0.92 | intron | DACT2 | U |
| 1305 | chr4 | 401616593 | 40162119 | Pancreatic Alpha cells | 0.3 | 0.93 | Intergenic | RHOH | U |
| 1306 | chr9 | 91940837 | 91941263 | Pancreatic Alpha cells | 0.26 | 0.88 | intron | SECISBP2 | U |
| 1307 | chr2 | 240362418 | 240362637 | Pancreatic Alpha cells | 0.93 | 0.17 | Intergenic | HDAC4 | M |
| 1308 | chr13 | 28492917 | 28492983 | Pancreatic Alpha cells | 0.88 | 0.13 | Intergenic | PDX1 | M |
| 1309 | chr13 | 28492638 | 28492765 | Pancreatic Alpha cells | 0.92 | 0.19 | Intergenic | PDX1 | M |
| 1310 | chr16 | 77823862 | 77824022 | Pancreatic Alpha cells | 0.86 | 0.16 | intron | VAT1L | M |
| 1311 | chr12 | 523461001 | 52346582 | Pancreatic Alpha cells | 0.75 | 0.07 | promoter-TSS | ACVR1B | M |
| 1312 | chr17 | 72348064 | 72348180 | Pancreatic Alpha cells | 0.78 | 0.11 | intron, exon | KIF19, KIF19 | M |
| 1313 | chr2 | 219910701 | 219910965 | Pancreatic Alpha cells | 0.76 | 0.1 | Intergenic | CCDC108 | M |
| 1314 | chr5 | 3278065 | 3278317 | Pancreatic Alpha cells | 0.77 | 0.17 | Intergenic | LOC285577 | M |
| 1315 | chr17 | 43044150 | 43044320 | Pancreatic Alpha cells | 0.6 | 0.12 | intron | C1QL1 | M |
| 1316 | chr10 | 26502618 | 26503037 | Pancreatic Alpha cells | 0.93 | 0.14 | Intergenic | GAD2 | M |
| 1317 | chr10 | 26501089 | 26501149 | Pancreatic Alpha cells | 0.96 | 0.14 | exon | MYO3A | M |
| 1318 | chr10 | 26500832 | 26501059 | Pancreatic Alpha cells | 0.94 | 0.15 | exon | MYO3A | M |
| 1319 | chr10 | 26500635 | 26500794 | Pancreatic Alpha cells | 0.9. | 0.13 | intron | MYO3A | M |
| 1320 | chr11 | 31846812 | 31846850 | Pancreatic Alpha cells | 0.9 | 0.19 | intron | DKFZp686K1684 | M |
| 1321 | chr | 134585146 | 134585371 | Pancreatic Alpha cells | 0.76 | 0.06 | Intergenic | ST3GAL1 | M |
| 1322 | chr19 | 41025447 | 41025747 | Pancreatic Alpha cells | 0.79 | 0.09 | exon | SPTBN4 | M |
| 1323 | chr7 | 157482024 | 157482301 | Pancreatic Alpha cells | 0.81 | 0.12 | intron | PTPRN2 | M |
| 1324 | chr7 | 157482641 | 157483120 | Pancreatic Alpha cells | 0.7 | 0.06 | intron | PTPRN2 | M |
| 1325 | chr16 | 77823437 | 77823725 | Pancreatic Alpha cells | 0.74 | 0.12 | intron | VAT1L | M |
| 1326 | chr19 | 6022436 | 6022549 | Pancreatic Alpha cells | 0.69 | 0.11 | intron | RFX2 | M |
| 1327 | chr19 | 38877908 | 38878227 | Pancreatic Alpha cells | 0.68 | 0.1 | intron | GGN | M |
| 1328 | chr3 | 1268546751 | 126854768 | Pancreatic Alpha cells | 0.66 | 0.09 | Intergenic | PLXNA1 | M |
| 1329 | chr10 | 99080071 | 99080335 | Pancreatic Alpha cells | 0.59 | 0.07 | exon | FRAT1 | M |
| 1330 | chr19 | 387188073 | 38718984 | Pancreatic Alpha cells | 0.63 | 0.13 | intron | DPF1 | M |
| 1331 | chr19 | 38877540 | 38877629 | Pancreatic Alpha cells | 0.61 | 0.17 | exon | GGN | M |
| 1332 | chr1 | 110175031 | 11017565 | Pancreatic Beta cells | 0.02 | 0.94 | intron | C1orf127 | U |
| 1333 | chr19 | 33035544 | 33035584 | Pancreatic Beta cells | 0.01 | 0.92 | Intergenic | PDCD5 | U |
| 1334 | chr17 | 45757584 | 45757963 | Pancreatic Beta cells | 0.02 | 0.93 | exon, intron | KPNB1 | U |
| 1335 | chr16 | 710495261 | 71049601 | Pancreatic Beta cells | 0 | 0.89 | intron | HYDIN | U |
| 1336 | chr5 | 45128099 | 45128339 | Pancreatic Beta cells | 0.04 | 0.92 | Intergenic | MRPS30 | U |
| 1337 | chr7 | 157822975 | 157823021 | Pancreatic Beta cells | 0.08 | 0.94 | intron | PTPRN2 | U |
| 1338 | chr12 | 21533849 | 21534202 | Pancreatic Beta cells | 0.02 | 0.88 | intron | SLCO1A2 | U |
| 1339 | chr7 | 111371216 | 111371582 | Pancreatic Beta cells | 0.06 | 0.91 | intron | DOCK4 | U |
| 1340 | chr19 | 4614761 | 4614944 | Pancreatic Beta cells | 0.07 | 0.91 | Intergenic | TNFAIP8L1 | U |
| 1341 | chr5 | 438160701 | 43316450 | Pancreatic Beta cells | 0.06 | 0.9 | Intergenic | HMGCS1 | U |
| 1342 | chr22 | 445512591 | 44551433 | Pancreatic Beta cells | 0.03 | 0.87 | intron | PARVB | U |
| 1343 | chr12 | 105694549 | 105694871 | Pancreatic Beta cells | 0.02 | 0.86 | Intergenic | C12orf75 | U |
| 1344 | chr10 | 94293775 | 94293990 | Pancreatic Beta cells | 0.1 | 0.92 | intron | IDE | U |
| 1345 | chr2 | 225897978 | 225898119 | Pancreatic Beta cells | 0.13 | 0.93 | intron | DOCK10 | U |
| 1346 | chr15 | 28473191 | 28473512 | Pancreatic Beta cells | 0.19 | 0.97 | exon, intron | HERC2 | U |
| 1347 | chr16 | 84091831 | 84092107 | Pancreatic Beta cells | 0.16 | 0.94 | intron | MBTPS1 | U |
| 1348 | chr11 | 46711310 | 46711664 | Pancreatic Beta cells | 0.22 | 0.94 | intron | ARHGAP2 | U |
| 1349 | chr22 | 47460066 | 47460436 | Pancreatic Beta cells | 0.22 | 0.93 | intron | TBC1D22A | U |
| 1350 | chr17 | 33443734 | 33444045 | Pancreatic Beta cells | 0.24 | 0.95 | exon, intron | RAD51D | U |
| 1351 | chr3 | 115762709 | 115762787 | Pancreatic Beta cells | 0.28 | 0.95 | intron | LSAMP | U |
| 1352 | chr2 | 239293858 | 293966 | Pancreatic Beta cells | 0.03 | 0.95 | intron | TRAF3IP1 | U |
| 1353 | chr8 | 131216273 | 131216600 | Pancreatic Beta cells | 0.02 | 0.94 | intron | ASAP1 | U |
| 1354 | chr1 | 10573884 | 10574070 | Pancreatic Beta cells | 0.01 | 0.91 | intron | PEX14 | U |
| 1355 | chr5 | 6639629 | 6639828 | Pancreatic Beta cells | 0.02 | 0.92 | intron | SRD5A1 | U |
| 1356 | chr4 | 1526193533 | 152619788 | Pancreatic Beta cells | 0.03 | 0.93 | intron | PET112 | U |
| 1357 | chr7 | 152472533 | 152472635 | Pancreatic Beta cells | 0.02 | 0.91 | intron | ACTR3B | U |
| 1358 | chr5 | 1377127613 | 137712926 | Pancreatic Beta cells | 0.02 | 0.91 | intron | KDM3B | U |
| 1359 | chr10 | 307424391 | 30742607 | Pancreatic Beta cells | 0.03 | 0.92 | intron | MAP3K8 | U |
| 1360 | chr8 | 123843945 | 123844168 | Pancreatic Beta cells | 0.05 | 0.93 | intron | ZHX2 | U |
| 1361 | chr2 | 1447151103 | 144715490 | Pancreatic Beta cells | 0.02 | 0.9 | intron | GTDC1 | U |
| 1362 | chr4 | 188074215 | 188074325 | Pancreatic Beta cells | 0.02 | 0.89 | Intergenic | LOC339975 | U |
| 1363 | chr1 | 11303087 | 11303223 | Pancreatic Beta cells | 0.04 | 0.91 | intron | MTOR | U |
| 1364 | chr13 | 52405059 | 52405168 | Pancreatic Beta cells | 0.03 | 0.89 | intron | LINC00282 | U |
| 1365 | chr2 | 34244021 | 3424584 | Pancreatic Beta cells | 0.03 | 0.89 | intron | TRAPPC12 | U |
| 1366 | chr3 | 172640814 | 172641119 | Pancreatic Beta cells | 0.03 | 0.89 | intron | SPATA16 | U |
| 1367 | chr6 | 81314161 | 81314268 | Pancreatic Beta cells | 0.06 | 0.91 | Intergenic | BCKDHB | U |
| 1368 | chr13 | 56573620 | 56573768 | Pancreatic Beta cells | 0.03 | 0.88 | Intergenic | MIR5007 | U |
| 1369 | chr2 | 233422665 | 233422859 | Pancreatic Beta cells | 0.05 | 0.9 | intron | EIF4E2 | U |
| 1370 | chr15 | 59577839 | 59578065 | Pancreatic Beta cells | 0.05 | 0.9 | intron | MYO1E | U |
| 1371 | chr1 | 238543890 | 238544336 | Pancreatic Beta cells | 0.03 | 0.88 | Intergenic | LOC339535 | U |
| 1372 | chr2 | 221134662 | 221134943 | Pancreatic Beta cells | 0.03 | 0.87 | Intergenic | MIR4268 | U |
| 1373 | chr4 | 184808460 | 184808821 | Pancreatic Beta cells | 0.05 | 0.89 | Intergenic | STOX2 | U |
| 1374 | chr11 | 2174558 | 2174640 | Pancreatic Beta cells | 0.06 | 0.89 | intron | INS-IGF2 | U |
| 1375 | chr5 | 1762609941 | 176261118 | Pancreatic Beta cells | 0.05 | 0.88 | intron | UNC5A | U |
| 1376 | chr8 | 99353984 | 99354240 | Pancreatic Beta cells | 0.06 | 0.89 | Intergenic | NIPAL2 | U |
| 1377 | chr1 | 11007326 | 11007615 | Pancreatic Beta cells | 0.06 | 0.89 | intron | C1orf127 | U |
| 1378 | chr15 | 33203375 | 33203671 | Pancreatic Beta cells | 0.1 | 0.93 | intron | FMN1 | U |
| 1379 | chr5 | 39002706 | 39002764 | Pancreatic Beta cells | 0.14 | 0.96 | exon | RICTOR | U |
| 1380 | chr16 | 71798505 | 71798635 | Pancreatic Beta cells | 0.12 | 0.94 | intron | AP1G1 | U |
| 1381 | chr3 | 72775909 | 72776073 | Pancreatic Beta cells | 0.04 | 0.86 | Intergenic | SHQ1 | U |
| 1382 | chr9 | 116745198 | 116745432 | Pancreatic Beta cells | 0.08 | 0.9 | intron | ZNF618 | U |
| 1383 | chr5 | 118554340 | 118554434 | Pancreatic Beta cells | 0.13 | 0.94 | intron | DMXL1 | U |
| 1384 | chr9 | 135887415 | 135887513 | Pancreatic Beta cells | 0.03 | 0.84 | Intergenic | GTF3C5 | U |
| 1385 | chr8 | 48809687 | 48809982 | Pancreatic Beta cells | 0.11 | 0.92 | exon | PRKDC | U |
| 1386 | chr2 | 99487624 | 99487975 | Pancreatic Beta cells | 0.07 | 0.88 | intron | KIAA1211L | U |
| 1387 | chr1 | 2016745801 | 201674721 | Pancreatic Beta cells | 0.09 | 0.89 | intron | NAV1 | U |
| 1388 | chr11 | 834063901 | 83406584 | Pancreatic Beta cells | 0.09 | 0.89 | intron | DLG2 | U |
| 1389 | chr2 | 2367575941 | 236757902 | Pancreatic Beta cells | 0.11 | 0.91 | intron | AGAP1 | U |
| 1390 | chr3 | 122144391 | 122144746 | Pancreatic Beta cells | 0.12 | 0.92 | exon | KPNA1 | U |
| 1391 | chr1 | 16030996 | 16031353 | Pancreatic Beta cells | 0.07 | 0.87 | intron | PLEKHM2 | U |
| 1392 | chr6 | 154919079 | 154919449 | Pancreatic Beta cells | 0.06 | 0.86 | Intergenic | CNKSR3 | U |
| 1393 | chr8 | 1421584211 | 142158610 | Pancreatic Beta cells | 0.11 | 0.9 | intron | DENND3 | U |
| 1394 | chr10 | 70010968 | 70011291 | Pancreatic Beta cells | 0.05 | 0.84 | Intergenic | ATOH7 | U |
| 1395 | chr16 | 5153423 | 5153815 | Pancreatic Beta cells | 0.06 | 0.85 | Intergenic | FAM86A | U |
| 1396 | chr19 | 43995336 | 43995731 | Pancreatic Beta cells | 0.12 | 0.91 | intron | PHLDB3 | U |
| 1397 | chr3 | 197212554 | 197212982 | Pancreatic Beta cells | 0.11 | 0.9 | Intergenic | BDH1 | U |
| 1398 | chr10 | 133058775 | 133058862 | Pancreatic Beta cells | 0.12 | 0.9 | intron | TCERG1L | U |
| 1399 | chr13 | 94650116 | 94650372 | Pancreatic Beta cells | 0.12 | 0.9 | intron | GPC6 | U |
| 1400 | chr7 | 70038370 | 70038661 | Pancreatic Beta cells | 0.11 | 0.89 | intron | AUTS2 | U |
| 1401 | chr14 | 101186014 | 101186336 | Pancreatic Beta cells | 0.04 | 0.82 | Intergenic | DLK1 | U |
| 1402 | chr10 | 35593985 | 35594172 | Pancreatic Beta cells | 0.13 | 0.9 | intron | CCNY | U |
| 1403 | chr10 | 94293767 | 94293991 | Pancreatic Beta cells | 0.15 | 0.92 | intron | IDE | U |
| 1404 | chr11 | 17482160 | 17482576 | Pancreatic Beta cells | 0.09 | 0.86 | exon, intron | ABCC8, ABCC8 | U |
| 1405 | chr18 | 545185613 | 54518665 | Pancreatic Beta cells | 0.14 | 0.9 | intron | WDR7 | U |
| 1406 | chr1 | 236198381 | 23619958 | Pancreatic Beta cells | 0.19 | 0.95 | Intergenic | HNRNPR | U |
| 1407 | chr1 | 236432042 | 236432165 | Pancreatic Beta cells | 0.13 | 0.89 | intron | ERO1LB | U |
| 1408 | chr2 | 66554646 | 66554819 | Pancreatic Beta cells | 0.12 | 0.88 | Intergenic | MIR4778 | U |
| 1409 | chr21 | 47735995 | 47736173 | Pancreatic Beta cells | 0.16 | 0.91 | intron | C21orf58 | U |
| 1410 | chr9 | 1346500033 | 134650240 | Pancreatic Beta cells | 0.07 | 0.82 | Intergenic | RAPGEF1 | U |
| 1411 | chr4 | 165846801 | 16584939 | Pancreatic Beta cells | 0.16 | 0.91 | intron | LDB2 | U |
| 1412 | chr17 | 67530192 | 67530481 | Pancreatic Beta cells | 0.15 | 0.9 | intron | MAP2K6 | U |
| 1413 | chr21 | 48078860 | 48079192 | Pancreatic Beta cells | 0.19 | 0.94 | exon | PRMT2 | U |
| 1414 | chr8 | 144555154 | 144555307 | Pancreatic Beta cells | 0.11 | 0.85 | intron | ZC3H3 | U |
| 1415 | chr3 | 14511595 | 14511792 | Pancreatic Beta cells | 0.09 | 0.83 | intron | SLC6A6 | U |
| 1416 | chr17 | 43091359 | 43091586 | Pancreatic Beta cells | 0.15 | 0.89 | Intergenic | C1QL1 | U |
| 1417 | chr20 | 57398590 | 57398840 | Pancreatic Beta cells | 0.11 | 0.85 | intron | GNAS-AS1 | U |
| 1418 | chr3 | 197431446 | 197431620 | Pancreatic Beta cells | 0.21 | 0.93 | exon | KIAA0226 | U |
| 1419 | chr8 | 74351022 | 74351241 | Pancreatic Beta cells | 0.17 | 0.9 | exon | STAU2-AS1 | U |
| 1420 | chr12 | 80402312 | 80402559 | Pancreatic Beta cells | 0.19 | 0.92 | Intergenic | PPP1R12A | U |
| 1421 | chr17 | 3737463 | 3737841 | Pancreatic Beta cells | 0.18 | 0.9 | intron | C17orf85 | U |
| 1422 | chr8 | 140900839 | 140901244 | Pancreatic Beta cells | 0.13 | 0.85 | intron | TRAPPC9 | U |
| 1423 | chr2 | 3431774 | 3432180 | Pancreatic Beta cells | 0.18 | 0.9 | intron | TRAPPC12 | U |
| 1424 | chr16 | 70619229 | 70619378 | Pancreatic Beta cells | 0.23 | 0.93 | intron | IL34 | U |
| 1425 | chr1 | 38058749 | 38058959 | Pancreatic Beta cells | 0.21 | 0.91 | intron | GNL2 | U |
| 1426 | chr8 | 144545917 | 144546130 | Pancreatic Beta cells | 0.19 | 0.89 | intron | ZC3H3 | U |
| 1427 | chr7 | 1293330233 | 129333347 | Pancreatic Beta cells | 0.24 | 0.94 | intron | NRF1 | U |
| 1428 | chr18 | 29493069 | 29493412 | Pancreatic Beta cells | 0.21 | 0.91 | exon, intron | TRAPPC8, TRAPPC8 | U |
| 1429 | chr1 | 111011211 | 111011645 | Pancreatic Beta cells | 0.13 | 0.83 | Intergenic | CYMP | U |
| 1430 | chr15 | 52008281 | 52008537 | Pancreatic Beta cells | 0.25 | 0.94 | intron | SCG3 | U |
| 1431 | chr10 | 121146663 | 121146920 | Pancreatic Beta cells | 0.23 | 0.92 | intron | GRK5 | U |
| 1432 | chr14 | 93400750 | 93401052 | Pancreatic Beta cells | 0.11 | 0.8 | intron | CHGA | U |
| 1433 | chr8 | 144578492 | 144578586 | Pancreatic Beta cells | 0.17 | 0.85 | intron | ZC3H3 | U |
| 1434 | chr18 | 72341539 | 72341718 | Pancreatic Beta cells | 0.23 | 0.91 | Intergenic | ZNF407 | U |
| 1435 | chr1 | 20826430 | 20826817 | Pancreatic Beta cells | 0.2 | 0.88 | exon | MUL1 | U |
| 1436 | chr1 | 110154279 | 110154578 | Pancreatic Beta cells | 0.25 | 0.92 | intron | GNAT2 | U |
| 1437 | chr14 | 103120423 | 103120620 | Pancreatic Beta cells | 0.3 | 0.94 | intron | RCOR1 | U |
| 1438 | chr3 | 118945649 | 118945808 | Pancreatic Beta cells | 0.33 | 0.94 | exon | B4GALT4 | U |
| 1439 | chr11 | 68181416 | 68181802 | Pancreatic Beta cells | 0.34 | 0.95 | intron | LRP5 | U |
| 1440 | chr7 | 156933820 | 156934217 | Pancreatic Beta cells | 0.32 | 0.93 | intron | UBE3C | U |
| 1441 | chr8 | 144546287 | 144546546 | Pancreatic Beta cells | 0.03 | 0.95 | intron | ZC3H3 | U |
| 1442 | chr22 | 32628870 | 32629026 | Pancreatic Beta cells | 0.02 | 0.9 | intron | SLC5A4 | U |
| 1443 | chr11 | 2176043 | 2176302 | Pancreatic Beta cells | 0.01 | 0.89 | intron | INS-IGF2 | U |
| 1444 | chr10 | 121550377 | 121550767 | Pancreatic Beta cells | 0.04 | 0.92 | intron | INPP5F | U |
| 1445 | chr18 | 56002609 | 56002896 | Pancreatic Beta cells | 0.1 | 0.9 | intron | NEDD4L | U |
| 1446 | chr10 | 135206703 | 135207007 | Pancreatic Beta cells | 0.02 | 0.94 | promoter-TSS | MTG1 | U |
| 1447 | chr6 | 11769090 | 11769210 | Pancreatic Beta cells | 0.05 | 0.93 | intron | ADTRP | U |
| 1448 | chr19 | 33953366 | 33953525 | Pancreatic Beta cells | 0.02 | 0.9 | intron | PEPD | U |
| 1449 | chr3 | 196100180 | 196100348 | Pancreatic Beta cells | 0.07 | 0.94 | intron | UBXN7 | U |
| 1450 | chr6 | 735859 | 736030 | Pancreatic Beta cells | 0.04 | 0.89 | Intergenic | EXOC2 | U |
| 1451 | chr22 | 30719173 | 30719481 | Pancreatic Beta cells | 0.05 | 0.9 | intron | TBC1D10A | U |
| 1452 | chr16 | 30958131 | 30958558 | Pancreatic Beta cells | 0.05 | 0.9 | exon | FBXL19 | U |
| 1453 | chr9 | 88873435 | 88873641 | Pancreatic Beta cells | 0.11 | 0.93 | intron | C9orf153 | U |
| 1454 | chr20 | 47604576 | 47604800 | Pancreatic Beta cells | 0.1 | 0.91 | intron | ARFGEF2 | U |
| 1455 | chr11 | 2176386 | 2176569 | Pancreatic Beta cells | 0.02 | 0.81 | intron | INS-IGF2 | U |
| 1456 | chr16 | 89202972 | 89203202 | Pancreatic Beta cells | 0.13 | 0.91 | promoter-TSS, exon, | ACSF3, ACSF3, ACSF3 | U |
| 1457 | chr9 | 101531696 | 101532118 | Pancreatic Beta cells | 0.16 | 0.92 | intron | ANKS6 | U |
| 1458 | chr15 | 994313401 | 99431443 | Pancreatic Beta cells | 0.21 | 0.94 | intron | IGF1R | U |
| 1459 | chr16 | 24794958 | 24795392 | Pancreatic Beta cells | 0.25 | 0.94 | intron | TNRC6A | U |
| 1460 | chr12 | 123826263 | 123826655 | Pancreatic Beta cells | 0.35 | 0.94 | intron | SBNO1 | U |
| 1461 | chr12 | 133196447 | 133196601 | Pancreatic Beta cells | 0.91 | 0.15 | intron | P2RX2 | M |
| 1462 | chr20 | 62105601 | 62105759 | Pancreatic Beta cells | 0.81 | 0.13 | Intergenic | KCNQ2 | M |
| 1463 | chr8 | 144502355 | 144502675 | Pancreatic Beta cells | 0.85 | 0.2 | Intergenic | MAFA | M |
| 1464 | chr8 | 136225451 | 136225753 | Pancreatic Beta cells | 0.86 | 0.22 | Intergenic | LOC286094 | M |
| 1465 | chr2 | 67877948 | 67878150 | Pancreatic Beta cells | 0.73 | 0.07 | Intergenic | ETAA1 | M |
| 1466 | chr11 | 33398566 | 33398747 | Pancreatic Beta cells | 0.78 | 0.18 | Intergenic | HIPK3 | M |
| 1467 | chr9 | 137981018 | 137981123 | Pancreatic Beta cells | 0.81 | 0.23 | intron | OLFM1 | M |
| 1468 | chr12 | 133196638 | 133196803 | Pancreatic Beta cells | 0.92 | 0.34 | intron | P2RX2 | M |
| 1469 | chr14 | 105266518 | 105266597 | Pancreatic Beta cells | 0.66 | 0.09 | promoter-TSS | ZBTB42 | M |
| 1470 | chr20 | 62105422 | 62105531 | Pancreatic Beta cells | 0.75 | 0.2 | Intergenic | KCNQ2 | M |
| 1471 | chr5 | 158476829 | 158477015 | Pancreatic Beta cells | 0.7 | 0.16 | intron | EBF1 | M |
| 1472 | chr2 | 219847327 | 219847685 | Pancreatic Beta cells | 0.91 | 0.11 | intron | FEV | M |
| 1473 | chr2 | 219847690 | 219848028 | Pancreatic Beta cells | 0.94 | 0.17 | intron | FEV | M |
| 1474 | chr2 | 1858523 | 1858835 | Pancreatic Beta cells | 0.84 | 0.1 | intron | MYT1L | M |
| 1475 | chr22 | 314805333 | 31480702 | Pancreatic Beta cells | 0.78 | 0.08 | promoter-TSS | SMTN | M |
| 1476 | chr1 | 2780356 | 2780689 | Pancreatic Beta cells | 0.88 | 0.21 | Intergenic | TTC34 | M |
| 1477 | chr15 | 53078206 | 53078705 | Pancreatic Beta cells | 0.77 | 0.13 | intron | ONECUT1 | M |
| 1478 | chr20 | 17183457 | 17183730 | Pancreatic Beta cells | 0.74 | 0.11 | Intergenic | PCSK2 | M |
| 1479 | chr12 | 71834853 | 71835077 | Pancreatic Beta cells | 0.66 | 0.08 | intron | LGR5 | M |
| 1480 | chr9 | 133320781 | 133321091 | Pancreatic Beta cells | 0.65 | 0.07 | intron | ASS1 | M |
| 1481 | chr20 | 61638977 | 61639325 | Pancreatic Beta cells | 0.69 | 0.11 | promoter-TSS | BHLHE23 | M |
| 1482 | chr20 | 568033223 | 56803388 | Pancreatic Beta cells | 0.66 | 0.13 | Intergenic | C20orf85 | M |
| 1483 | chr18 | 24130764 | 24131040 | Pancreatic Beta cells | 0.61 | 0.08 | promoter-TSS, intro [symbol] | KCTD1, KCTD1 | M |
| 1484 | chr1 | 226069476 | 226069831 | Pancreatic Beta cells | 0.55 | 0.03 | intron | TMEM63A | M |
| 1485 | chr19 | 17717375 | 17717593 | Pancreatic Beta cells | 0.57 | 0.07 | intron | UNC13A | M |
| 1486 | chr7 | 101575497 | 101575708 | Pancreatic Delta cells | 0 | 0.92 | intron | CUX1 | U |
| 1487 | chr17 | 55693892 | 55694221 | Pancreatic Delta cells | 0.05 | 0.94 | intron | MSI2 | U |
| 1488 | chr16 | 85546140 | 85546413 | Pancreatic Delta cells | 0.02 | 0.9 | Intergenic | GSE1 | U |
| 1489 | chr9 | 130226701 | 130226897 | Pancreatic Delta cells | 0.06 | 0.94 | intron | LRSAM1 | U |
| 1490 | chr13 | 49762704 | 49763099 | Pancreatic Delta cells | 0.06 | 0.91 | exon, intron | FNDC3A | U |
| 1491 | chr3 | 39497339 | 39497425 | Pancreatic Delta cells | 0.09 | 0.92 | Intergenic | MOBP | U |
| 1492 | chr6 | 37693027 | 37693154 | Pancreatic Delta cells | 0.07 | 0.9 | Intergenic | MDGA1 | U |
| 1493 | chr1 | 6019318 | 6019394 | Pancreatic Delta cells | 0.12 | 0.93 | intron | NPHP4 | U |
| 1494 | chr7 | 129945291 | 129945513 | Pancreatic Delta cells | 0.02 | 0.83 | intron | CPA4 | U |
| 1495 | chr17 | 33217450 | 33217683 | Pancreatic Delta cells | 0.04 | 0.85 | Intergenic | CCT6B | U |
| 1496 | chr5 | 170039130 | 170039386 | Pancreatic Delta cells | 0.08 | 0.88 | intron | KCNIP1 | U |
| 1497 | chr3 | 172058903 | 172059232 | Pancreatic Delta cells | 0.13 | 0.91 | intron | FNDC3B | U |
| 1498 | chr1 | 184078999 | 184079424 | Pancreatic Delta cells | 0.13 | 0.91 | Intergenic | TSEN15 | U |
| 1499 | chr9 | 129143335 | 129143780 | Pancreatic Delta cells | 0.11 | 0.89 | intron | MVB12B | U |
| 1500 | chr16 | 9663477 | 9663527 | Pancreatic Delta cells | 0.14 | 0.91 | Intergenic | MIR548X | U |
| 1501 | chr11 | 78039188 | 78039579 | Pancreatic Delta cells | 0.16 | 0.92 | intron | GAB2 | U |
| 1502 | chr13 | 43316442 | 43316643 | Pancreatic Delta cells | 0.18 | 0.91 | Intergenic | FAM216B | U |
| 1503 | chr4 | 152903957 | 152904377 | Pancreatic Delta cells | 0.13 | 0.86 | Intergenic | PET112 | U |
| 1504 | chr7 | 101575751 | 101576236 | Pancreatic Delta cells | 0.26 | 0.94 | intron | CUX1 | U |
| 1505 | chr2 | 39474855 | 39475300 | Pancreatic Delta cells | 0.24 | 0.91 | Intergenic | CDKL4 | U |
| 1506 | chr7 | 151850900 | 151851398 | Pancreatic Delta cells | 0.25 | 0.92 | exon | MLL3 | U |
| 1507 | chr7 | 69911307 | 69911583 | Pancreatic Delta cells | 0.28 | 0.94 | intron | AUTS2 | U |
| 1508 | chr5 | 54045100 | 54045465 | Pancreatic Delta cells | 0.25 | 0.9 | Intergenic | SNX18 | U |
| 1509 | chr3 | 187592073 | 187592343 | Pancreatic Delta cells | 0.05 | 0.91 | Intergenic | BCL6 | U |
| 1510 | chr1 | 107951331 | 107951610 | Pancreatic Delta cells | 0.05 | 0.89 | intron | NTNG1 | U |
| 1511 | chr1 | 29379317 | 29379780 | Pancreatic Delta cells | 0.06 | 0.89 | intron | EPB41 | U |
| 1512 | chr16 | 966029 | 966166 | Pancreatic Delta cells | 0.04 | 0.86 | intron | LMF1 | U |
| 1513 | chr18 | 2950356 | 2950609 | Pancreatic Delta cells | 0.13 | 0.92 | intron | LPIN2 | U |
| 1514 | chr16 | 17551988 | 17552088 | Pancreatic Delta cells | 0.02 | 0.83 | intron | XYLT1 | U |
| 1515 | chr11 | 63912203 | 63912368 | Pancreatic Delta cells | 0.09 | 0.89 | intron | MACROD1 | U |
| 1516 | chr10 | 33401284 | 33401567 | Pancreatic Delta cells | 0.07 | 0.87 | Intergenic | ITGB1 | U |
| 1517 | chr7 | 129945615 | 129945685 | Pancreatic Delta cells | 0.13 | 0.91 | intron | CPA4 | U |
| 1518 | chr4 | 187535391 | 187535469 | Pancreatic Delta cells | 0.08 | 0.86 | exon | FAT1 | U |
| 1519 | chr10 | 21239090 | 21239277 | Pancreatic Delta cells | 0.07 | 0.85 | intron | NEBL | U |
| 1520 | chr1 | 41841569 | 41841909 | Pancreatic Delta cells | 0.09 | 0.87 | Intergenic | EDN2 | U |
| 1521 | chr11 | 63835331 | 63835490 | Pancreatic Delta cells | 0.04 | 0.81 | intron | MACROD1 | U |
| 1522 | chr16 | 17493407 | 17493569 | Pancreatic Delta cells | 0.13 | 0.89 | intron | XYLT1 | U |
| 1523 | chr7 | 12645693 | 12645872 | Pancreatic Delta cells | 0.09 | 0.85 | intron | SCIN | U |
| 1524 | chr22 | 48478698 | 48478899 | Pancreatic Delta cells | 0.12 | 0.88 | Intergenic | MIR3201 | U |
| 1525 | chr16 | 69941097 | 69941324 | Pancreatic Delta cells | 0.11 | 0.87 | intron | WWP2 | U |
| 1526 | chr6 | 168560246 | 168560479 | Pancreatic Delta cells | 0.15 | 0.91 | Intergenic | FRMD1 | U |
| 1527 | chr1 | 38707894 | 38708273 | Pancreatic Delta cells | 0.1 | 0.86 | Intergenic | LOC339442 | U |
| 1528 | chr2 | 42565737 | 42565966 | Pancreatic Delta cells | 0.15 | 0.9 | Intergenic | COX7A2L | U |
| 1529 | chr16 | 87967699 | 87967816 | Pancreatic Delta cells | 0.11 | 0.85 | intron | CA5A | U |
| 1530 | chr2 | 9419562 | 9419748 | Pancreatic Delta cells | 0.13 | 0.87 | intron | ASAP2 | U |
| 1531 | chr26 | 86556120 | 86556455 | Pancreatic Delta cells | 0.16 | 0.9 | Intergenic | AGBL1 | U |
| 1532 | chr10 | 77717495 | 77717845 | Pancreatic Delta cells | 0.13 | 0.86 | intron | C10orf11 | U |
| 1533 | chr6 | 148789420 | 148789781 | Pancreatic Delta cells | 0.11 | 0.83 | intron | SASH1 | U |
| 1534 | chr18 | 34140963 | 34141439 | Pancreatic Delta cells | 0.14 | 0.87 | intron | FHOD3 | U |
| 1535 | chr3 | 187385076 | 187385216 | Pancreatic Delta cells | 0.12 | 0.83 | Intergenic | SST | U |
| 1536 | chr7 | 31869005 | 31869364 | Pancreatic Delta cells | 0.13 | 0.83 | intron | PDE1C | U |
| 1537 | chr10 | 131667822 | 131667900 | Pancreatic Delta cells | 0.17 | 0.86 | intron | EBF3 | U |
| 1538 | chr4 | 8034277 | 8034475 | Pancreatic Delta cells | 0.15 | 0.84 | exon | ABLIM2 | U |
| 1539 | chr4 | 169589443 | 169589857 | Pancreatic Delta cells | 0.21 | 0.9 | intron | PALLD | U |
| 1540 | chr16 | 75368216 | 75368300 | Pancreatic Delta cells | 0.2 | 0.88 | intron | CFDP1 | U |
| 1541 | chr4 | 102230945 | 102231107 | Pancreatic Delta cells | 0.2 | 0.88 | intron | PPP3CA | U |
| 1542 | chr7 | 155475836 | 155476284 | Pancreatic Delta cells | 0.23 | 0.91 | intron | RBM33 | U |
| 1543 | chr8 | 10149407 | 10149581 | Pancreatic Delta cells | 0.21 | 0.88 | intron | MSRA | U |
| 1544 | chr3 | 1511425393 | 151142620 | Pancreatic Delta cells | 0.23 | 0.89 | intron | MED12L | U |
| 1545 | chr12 | 118852674 | 118852833 | Pancreatic Delta cells | 0.25 | 0.91 | exon | SUDS3 | U |
| 1546 | chr15 | 61383738 | 61383947 | Pancreatic Delta cells | 0.22 | 0.87 | intron | RORA | U |
| 1547 | chr17 | 71315786 | 71316035 | Pancreatic Delta cells | 0.26 | 0.91 | Intergenic | CDC42EP4 | U |
| 1548 | chr6 | 158137642 | 158138002 | Pancreatic Delta cells | 0.2 | 0.85 | Intergenic | SNX9 | U |
| 1549 | chr6 | 41643405 | 41643574 | Pancreatic Delta cells | 0.18 | 0.82 | Intergenic | MDFI | U |
| 1550 | chr2 | 129168742 | 129169054 | Pancreatic Delta cells | 0.28 | 0.91 | Intergenic | HS6ST1 | U |
| 1551 | chr12 | 127371160 | 127371487 | Pancreatic Delta cells | 0.17 | 0.8 | Intergenic | LOC440117 | U |
| 1552 | chr3 | 54250346 | 54250790 | Pancreatic Delta cells | 0.21 | 0.83 | intron | CACNA2D3 | U |
| 1553 | chr15 | 70636344 | 70636535 | Pancreatic Delta cells | 0.29 | 0.9 | Intergenic | TLE3 | U |
| 1554 | chr11 | 117572066 | 117572324 | Pancreatic Delta cells | 0.27 | 0.88 | intron | DSCAML1 | U |
| 1555 | chr4 | 41786674 | 41787000 | Pancreatic Delta cells | 0.26 | 0.87 | Intergenic | PHOX2B | U |
| 1556 | chr16 | 2527294 | 2527627 | Pancreatic Delta cells | 0.3 | 0.9 | intron | TBC1D24 | U |
| 1557 | chr12 | 3048985 | 3049407 | Pancreatic Delta cells | 0.31 | 0.91 | exon | TULP3 | U |
| 1558 | chr1 | 203980720 | 203980834 | Pancreatic Delta cells | 0.21 | 0.8 | Intergenic | LINC00303 | U |
| 1559 | chr11 | 1174213473 | 117421479 | Pancreatic Delta cells | 0.25 | 0.84 | intron | DSCAML1 | U |
| 1560 | chr5 | 127246456 | 127246874 | Pancreatic Delta cells | 0.23 | 0.82 | Intergenic | RSPO3 | U |
| 1561 | chr7 | 6156468 | 6156952 | Pancreatic Delta cells | 0.3 | 0.89 | intron | USP42 | U |
| 1562 | chr3 | 13018910 | 13019406 | Pancreatic Delta cells | 0.24 | 0.83 | intron | IQSEC1 | U |
| 1563 | chr19 | 4729206 | 4729316 | Pancreatic Delta cells | 0.25 | 0.83 | Intergenic | DPP9 | U |
| 1564 | chr8 | 53214548 | 53214717 | Pancreatic Delta cells | 0.24 | 0.82 | intron | ST18 | U |
| 1565 | chr2 | 2422312113 | 242231529 | Pancreatic Delta cells | 0.31 | 0.89 | intron | HDLBP | U |
| 1566 | chr5 | 16844110 | 16844483 | Pancreatic Delta cells | 0.31 | 0.89 | intron | MYO10 | U |
| 1567 | chr15 | 61912008 | 61912385 | Pancreatic Delta cells | 0.22 | 0.8 | Intergenic | VPS13C | U |
| 1568 | chr17 | 8470034 | 8470527 | Pancreatic Delta cells | 0.35 | 0.93 | intron | MYH10 | U |
| 1569 | chr7 | 104887719 | 104887874 | Pancreatic Delta cells | 0.31 | 0.88 | intron | SRPK2 | U |
| 1570 | chr8 | 3556138 | 3556352 | Pancreatic Delta cells | 0.24 | 0.81 | intron | CSMD1 | U |
| 1571 | chr7 | 33827647 | 33827954 | Pancreatic Delta cells | 0.29 | 0.86 | Intergenic | BMPER | U |
| 1572 | chr10 | 72975603 | 72975916 | Pancreatic Delta cells | 0.25 | 0.82 | intron | UNC5B | U |
| 1573 | chr2 | 129592222 | 129592588 | Pancreatic Delta cells | 0.24 | 0.81 | Intergenic | HS6ST1 | U |
| 1574 | chr1 | 33887399 | 33887516 | Pancreatic Delta cells | 0.31 | 0.87 | Intergenic | PHC2 | U |
| 1575 | chr2 | 108362136 | 108362528 | Pancreatic Delta cells | 0.29 | 0.85 | Intergenic | OC729121 | U |
| 1576 | chr5 | 158696274 | 158696740 | Pancreatic Delta cells | 0.37 | 0.93 | intron | UBLCP1 | U |
| 1577 | chr1 | 224810551 | 224811050 | Pancreatic Delta cells | 0.31 | 0.87 | intron | CNIH3 | U |
| 1578 | chr10 | 13553787 | 13553990 | Pancreatic Delta cells | 0.34 | 0.89 | Intergenic | BEND7 | U |
| 1579 | chr1 | 2081445493 | 208144892 | Pancreatic Delta cells | 0.35 | 0.9 | Intergenic | CD34 | U |
| 1580 | chr9 | 15453796 | 15454134 | Pancreatic Delta cells | 0.35 | 0.89 | intron | SNAPC3 | U |
| 1581 | chr16 | 272580071 | 27258260 | Pancreatic Delta cells | 0.33 | 0.86 | intron | NSMCE1 | U |
| 1582 | chr12 | 463529663 | 46353303 | Pancreatic Delta cells | 0.35 | 0.88 | intron | SCAF11 | U |
| 1583 | chr7 | 99616252 | 99616659 | Pancreatic Delta cells | 0.35 | 0.88 | intron | ZKSCAN1 | U |
| 1584 | chr15 | 94958699 | 94959197 | Pancreatic Delta cells | 0.31 | 0.84 | intron | MCTP2 | U |
| 1585 | chr16 | 88895451 | 88895554 | Pancreatic Delta cells | 0.3 | 0.82 | intron | GALNS | U |
| 1586 | chr2 | 2196875593 | 219687848 | Pancreatic Delta cells | 0.34 | 0.86 | exon | PRKAG3 | U |
| 1587 | chr10 | 16566692 | 16567000 | Pancreatic Delta cells | 0.35 | 0.87 | Intergenic | C1QL3 | U |
| 1588 | chr10 | 21246174 | 21246623 | Pancreatic Delta cells | 0.37 | 0.89 | intron | NEBL | U |
| 1589 | chr11 | 723741201 | 72374212 | Pancreatic Delta cells | 0.32 | 0.83 | intron | PDE2A | U |
| 1590 | chr2 | 89339816 | 89340006 | Pancreatic Delta cells | 0.28 | 0.79 | Intergenic | MIR4436A | U |
| 1591 | chr3 | 72949112 | 72949403 | Pancreatic Delta cells | 0.35 | 0.86 | intron | GXYLT2 | U |
| 1592 | chr8 | 88901809 | 88902151 | Pancreatic Delta cells | 0.33 | 0.84 | Intergenic | DCAF4L2 | U |
| 1593 | chr13 | 1135843483 | 113584427 | Pancreatic Delta cells | 0.37 | 0.86 | Intergenic | MCF2L-AS1 | U |
| 1594 | chr11 | 13197303 | 13197521 | Pancreatic Delta cells | 0.35 | 0.84 | Intergenic | ARNTL | U |
| 1595 | chr9 | 133740366 | 133740764 | Pancreatic Delta cells | 0.19 | 0.88 | intron | ABL1 | U |
| 1596 | chr12 | 121439119 | 121439260 | Pancreatic Delta cells | 0.29 | 0.85 | exon | HNF1A | U |
| 1597 | chr1 | 168260481 | 168260698 | Pancreatic Delta cells | 0.07 | 0.95 | exon | TBX19 | U |
| 1598 | chr20 | 20412544 | 20412857 | Pancreatic Delta cells | 0.16 | 0.9 | intron | RALGAPA2 | U |
| 1599 | chr1 | 230393372 | 230393593 | Pancreatic Delta cells | 0.07 | 0.93 | intron | GALNT2 | U |
| 1600 | chr9 | 83639823 | 83640085 | Pancreatic Delta cells | 0.04 | 0.85 | Intergenic | TLE1 | U |
| 1601 | chr20 | 20417198 | 20417391 | Pancreatic Delta cells | 0.08 | 0.88 | intron | RALGAPA2 | U |
| 1602 | chr1 | 6329539 | 6329695 | Pancreatic Delta cells | 0.1 | 0.84 | intron | ACOT7 | U |
| 1603 | chr12 | 130953864 | 130954237 | Pancreatic Delta cells | 0.1 | 0.84 | intron | RIMBP2 | U |
| 1604 | chr4 | 6974639 | 6974856 | Pancreatic Delta cells | 0.13 | 0.86 | intron | TBC1D14 | U |
| 1605 | chr6 | 3143797 | 3143899 | Pancreatic Delta cells | 0.17 | 0.89 | intron | BPHL | U |
| 1606 | chr5 | 10986780 | 10986974 | Pancreatic Delta cells | 0.09 | 0.79 | intron | CTNND2 | U |
| 1607 | chr11 | 72022054 | 72022343 | Pancreatic Delta cells | 0.21 | 0.89 | intron | CLPB | U |
| 1608 | chr14 | 76641864 | 76642294 | Pancreatic Delta cells | 0.24 | 0.92 | intron | GPATCH2L | U |
| 1609 | chr14 | 42359802 | 42359905 | Pancreatic Delta cells | 0.25 | 0.91 | intron | LRFN5 | U |
| 1610 | chr20 | 41198544 | 41198903 | Pancreatic Delta cells | 0.28 | 0.87 | intron | PTPRT | U |
| 1611 | chr14 | 25700895 | 25701112 | Pancreatic Delta cells | 0.31 | 0.88 | Intergenic | STXBP6 | U |
| 1612 | chr8 | 11300158 | 11300372 | Pancreatic Delta cells | 0.36 | 0.9 | intron | FAM167A | U |
| 1613 | chr9 | 97152452 | 97152730 | Pancreatic Delta cells | 0.35 | 0.83 | intron | HIATL1 | U |
| 1614 | chr17 | 74905997 | 74906231 | Pancreatic Delta cells | 0.86 | 0.05 | intron | MGAT5B | M |
| 1615 | chr5 | 138289132 | 138289385 | Pancreatic Delta cells | 0.87 | 0.06 | intron | SIL1 | M |
| 1616 | chr16 | 51188740 | 51188814 | Pancreatic Delta cells | 0.86 | 0.22 | Intergenic | SALL1 | M |
| 1617 | chr10 | 21581747 | 21582140 | Pancreatic Delta cells | 0.66 | 0.06 | Intergenic | NEBL-AS1 | M |
| 1618 | chr6 | 170602017 | 170602485 | Pancreatic Delta cell: | 0.73 | 0.13 | Intergenic | DLL1 | M |
| 1619 | chr9 | 14321662 | 14322059 | Pancreatic Delta cells | 0.72 | 0.13 | intron | NFIB | M |
| 1620 | chr2 | 2185180281 | 218518255 | Pancreatic Delta cells | 0.74 | 0.2 | intron | DIRC3 | M |
| 1621 | chr17 | 61600872 | 61601019 | Pancreatic Delta cells | 0.68 | 0.15 | promoter-TSS, intron | KCNH6, KCNH6 | M |
| 1622 | chr5 | 50760907 | 50761202 | Pancreatic Delta cells | 0.73 | 0.19 | Intergenic | LOC642366 | M |
| 1623 | chr11 | 72353008 | 72353158 | Pancreatic Delta cells | 0.63 | 0.11 | intron | PDE2A | M |
| 1624 | chr5 | 162992362 | 162992634 | Pancreatic Delta cells | 0.65 | 0.19 | Intergenic | MAT2B | M |
| 1625 | chr17 | 29297437 | 29297654 | Pancreatic Delta cells | 0.55 | 0.06 | promoter-TSS | RNF135 | M |
| 1626 | chr22 | 25815475 | 25815847 | Pancreatic Delta cells | 0.86 | 0.12 | Intergenic | CRYBB2P1 | M |
| 1627 | chr10 | 94828878 | 94829010 | Pancreatic Delta cells | 0.9 | 0.17 | TTS | CYP26C1 | M |
| 1628 | chr5 | 176038455 | 176038608 | Pancreatic Delta cells | 0.76 | 0.04 | Intergenic | GPRIN1 | M |
| 1629 | chr10 | 94825641 | 94825718 | Pancreatic Delta cells | 0.78 | 0.08 | intron | CYP26C3 | M |
| 1630 | chr10 | 94828683 | 94828872 | Pancreatic Delta cells | 0.77 | 0.09 | TTS | CYP26C1 | M |
| 1631 | chr17 | 29721727 | 29721957 | Pancreatic Delta cells | 0.78 | 0.1 | intron | RAB11FIP4 | M |
| 1632 | chr11 | 31848747 | 31848829 | Pancreatic Delta cells | 0.83 | 0.14 | intron | DKFZp686K1684 | M |
| 1633 | chr7 | 156804561 | 156804851 | Pancreatic Delta cells | 0.72 | 0.11 | intron | LOC645249 | M |
| 1634 | chr19 | 11532740 | 11532885 | Pancreatic Delta cells | 0.78 | 0.19 | intron, exon | CCDC151, CCDC151 | M |
| 1635 | chr16 | 51190085 | 51190396 | Pancreatic Delta cells | 0.67 | 0.08 | Intergenic | SALL1 | M |
| 1636 | chr6 | 34110323 | 34110618 | Pancreatic Delta cells | 0.65 | 0.1 | intron | GRM4 | M |
| 1637 | chr20 | 21488274 | 21488427 | Pancreatic Delta cells | 0.61 | 0.1 | Intergenic | NKX2-2 | M |
| 1638 | chr11 | 113346279 | 113346492 | Pancreatic Delta cells | 0.53 | 0.09 | exon, promoter-TSS | DRD2, DRD2 | M |
| 1639 | chr5 | 133081567 | 133081764 | Pancreatic Ductal cells | 0.04 | 0.91 | Intergenic | FSTL4 | U |
| 1640 | chr7 | 157290520 | 157290591 | Pancreatic Ductal cells | 0.07 | 0.93 | Intergenic | MIR153-2 | U |
| 1641 | chr21 | 37749203 | 37749408 | Pancreatic Ductal cells | 0.04 | 0.9 | TTS | MORC3 | U |
| 1642 | chr7 | 156781826 | 156781901 | Pancreatic Ductal cells | 0.05 | 0.9 | Intergenic | MNX1 | U |
| 1643 | chr4 | 72091592 | 72091932 | Pancreatic Ductal cells | 0.04 | 0.89 | intron | SLC4A4 | U |
| 1644 | chr2 | 97169667 | 97170012 | Pancreatic Ductal cells | 0.04 | 0.88 | intron | NEURL3 | U |
| 1645 | chr13 | 52359350 | 52359736 | Pancreatic Ductal cells | 0.07 | 0.9 | intron | DHRS12 | U |
| 1646 | chr4 | 72074004 | 72074142 | Pancreatic Ductal cells | 0.12 | 0.95 | intron | SLC4A4 | U |
| 1647 | chr16 | 85230631 | 85230804 | Pancreatic Ductal cells | 0.05 | 0.87 | Intergenic | LOC400548 | U |
| 1648 | chr21 | 41039727 | 41040109 | Pancreatic Ductal cells | 0.11 | 0.92 | Intergenic | B3GALT5 | U |
| 1649 | chr10 | 114877816 | 114878048 | Pancreatic Ductal cells | 0.12 | 0.92 | intron | TCF7L2 | U |
| 1650 | chr4 | 72065389 | 72065647 | Pancreatic Ductal cells | 0.13 | 0.92 | intron | SLC4A4 | U |
| 1651 | chr17 | 33419036 | 33419439 | Pancreatic Ductal cells | 0.08 | 0.87 | intron | RADS1L3-RFFL | U |
| 1652 | chr4 | 72081451 | 72081905 | Pancreatic Ductal cells | 0.13 | 0.91 | intron | SLC4A4 | U |
| 1653 | chr11 | 128609785 | 128609919 | Pancreatic Ductal cells | 0.05 | 0.83 | intron | FLI1 | U |
| 1654 | chr8 | 97017363 | 97017718 | Pancreatic Ductal cells | 0.11 | 0.87 | Intergenic | LOC100500773 | U |
| 1655 | chr2 | 8478761 | 8479008 | Pancreatic Ductal cells | 0.14 | 0.89 | Intergenic | LINC00299 | U |
| 1656 | chr1 | 596362101 | 59636650 | Pancreatic Ductal cells | 0.13 | 0.88 | Intergenic | HSD52 | U |
| 1657 | chr1 | 110202186 | 110202252 | Pancreatic Ductal cells | 0.12 | 0.86 | intron | GSTM4 | U |
| 1658 | chr4 | 72090796 | 72091063 | Pancreatic Ductal cells | 0.21 | 0.94 | intron | SLC4A4 | U |
| 1659 | chr10 | 134471465 | 134471585 | Pancreatic Ductal cells | 0.02 | 0.92 | intron | INPP5A | U |
| 1660 | chr3 | 570256731 | 57025761 | Pancreatic Ductal cells | 0.02 | 0.87 | intron | ARHGEF3 | U |
| 1661 | chr2 | 99149523 | 99149931 | Pancreatic Ductal cells | 0.05 | 0.9 | intron | INPP4A | U |
| 1662 | chr5 | 66107801 | 66108252 | Pancreatic Ductal cells | 0.04 | 0.89 | intron | MAST4 | U |
| 1663 | chr1 | 179264758 | 179265022 | Pancreatic Ductal cells | 0.06 | 0.9 | intron | SOAT1 | U |
| 1664 | chr16 | 4039211 | 4039416 | Pancreatic Ductal cells | 0.07 | 0.9 | intron | ADCY9 | U |
| 1665 | chr3 | 29471784 | 29471850 | Pancreatic Ductal cells | 0.08 | 0.9 | intron | RBMS3 | U |
| 1666 | chr7 | 47633278 | 47633455 | Pancreatic Ductal cells | 0.08 | 0.9 | Intergenic | TNS3 | U |
| 1667 | chr4 | 106834079 | 106834276 | Pancreatic Ductal cells | 0.08 | 0.9 | intron | NPNT | U |
| 1668 | chr2 | 106553512 | 106553931 | Pancreatic Ductal cells | 0.05 | 0.87 | Intergenic | C2orf40 | U |
| 1669 | chr4 | 72062018 | 72062500 | Pancreatic Ductal cells | 0.04 | 0.86 | intron | SLC4A4 | U |
| 1670 | chr16 | 3505114 | 3505177 | Pancreatic Ductal cells | 0.08 | 0.89 | intron | NAA60 | U |
| 1671 | chr12 | 121603114 | 121603241 | Pancreatic Ductal cells | 0.05 | 0.86 | intron | P2RX7 | U |
| 1672 | chr11 | 2751716 | 2752023 | Pancreatic Ductal cells | 0.05 | 0.86 | intron | KCNQ1 | U |
| 1673 | chr4 | 24102403 | 24102767 | Pancreatic Ductal cells | 0.06 | 0.87 | Intergenic | PPARGC1A | U |
| 1674 | chr2 | 99409519 | 99409597 | Pancreatic Ductal cells | 0.07 | 0.87 | TTS, Intergenic | KIAA1211L, MGAT4A | U |
| 1675 | chr2 | 102727793 | 102727894 | Pancreatic Ductal cells | 0.08 | 0.88 | Intergenic | IL1R1 | U |
| 1676 | chr13 | 52359408 | 52359737 | Pancreatic Ductal cells | 0.07 | 0.87 | intron | DHRS12 | U |
| 1677 | chr17 | 41800032 | 41800495 | Pancreatic Ductal cells | 0.02 | 0.82 | Intergenic | SOST | U |
| 1678 | chr2 | 171086612 | 171086882 | Pancreatic Ductal cells | 0.06 | 0.85 | intron | MYO3B | U |
| 1679 | chr10 | 117842780 | 117842934 | Pancreatic Ductal cells | 0.04 | 0.82 | intron | GFRA1 | U |
| 1680 | chr10 | 13772059 | 13772243 | Pancreatic Ductal cells | 0.03 | 0.81 | intron | FRMD4A | U |
| 1681 | chr4 | 187225006 | 187225234 | Pancreatic Ductal cells | 0.06 | 0.84 | intron | LOC285441 | U |
| 1682 | chr6 | 15861691 | 158617141 | Pancreatic Ductal cells | 0.13 | 0.91 | exon | GTF2H5 | U |
| 1683 | chr19 | 5603471 | 5603707 | Pancreatic Ductal cells | 0.11 | 0.89 | intron | SAFB2 | U |
| 1684 | chr12 | 110788669 | 110788963 | Pancreatic Ductal cells | 0.11 | 0.89 | TTS | ATP2A2 | U |
| 1685 | chr8 | 40026598 | 40026940 | Pancreatic Ductal cells | 0.09 | 0.87 | Intergenic | C8orf4 | U |
| 1686 | chr2 | 242565207 | 242565596 | Pancreatic Ductal cells | 0.13 | 0.91 | intron | THAP4 | U |
| 1687 | chr4 | 1362928 | 1363237 | Pancreatic Ductal cells | 0.12 | 0.89 | intron | UVSSA | U |
| 1688 | chr15 | 93874000 | 93874072 | Pancreatic Ductal cells | 0.14 | 0.9 | Intergenic | RGMA | U |
| 1689 | chr4 | 1780269 | 1780355 | Pancreatic Ductal cells | 0.08 | 0.84 | Intergenic | FGFR3 | U |
| 1690 | chr21 | 46744900 | 46745000 | Pancreatic Ductal cells | 0.06 | 0.82 | Intergenic | LOC642852 | U |
| 1691 | chr16 | 15984709 | 15984844 | Pancreatic Ductal cells | 0.12 | 0.88 | Intergenic | FOPNL | U |
| 1692 | chr4 | 1840046871 | 184004870 | Pancreatic Ductal cells | 0.15 | 0.9 | Intergenic | WWC2-AS2 | U |
| 1693 | chr10 | 134471997 | 134472211 | Pancreatic Ductal cells | 0.16 | 0.91 | intron | INPP5A | U |
| 1694 | chr4 | 72135052 | 72135293 | Pancreatic Ductal cells | 0.09 | 0.84 | intron | SLC4A4 | U |
| 1695 | chr10 | 30458261 | 30458756 | Pancreatic Ductal cells | 0.11 | 0.86 | Intergenic | KIAA1462 | U |
| 1696 | chr7 | 156781726 | 156781798 | Pancreatic Ductal cells | 0.04 | 0.78 | Intergenic | MNX1 | U |
| 1697 | chr4 | 72171381 | 72171508 | Pancreatic Ductal cells | 0.15 | 0.89 | intron | SLC4A4 | U |
| 1698 | chr14 | 105454616 | 105454780 | Pancreatic Ductal cells | 0.13 | 0.87 | intron | C14orf79 | U |
| 1699 | chr8 | 1237316521 | 123731880 | Pancreatic Ductal cells | 0.17 | 0.9 | Intergenic | ZHX2 | U |
| 1700 | chr2 | 129829412 | 129829688 | Pancreatic Ductal cells | 0.13 | 0.86 | Intergenic | HS6ST1 | U |
| 1701 | chr5 | 76136006 | 76136302 | Pancreatic Ductal cells | 0.09 | 0.82 | Intergenic | S100Z | U |
| 1702 | chr6 | 158615276 | 158615598 | Pancreatic Ductal cells | 0.09 | 0.82 | exon | GTF2H5 | U |
| 1703 | chr3 | 128909292 | 128909617 | Pancreatic Ductal cells | 0.18 | 0.91 | Intergenic | CNBP | U |
| 1704 | chr15 | 93830808 | 93831210 | Pancreatic Ductal cells | 0.16 | 0.89 | Intergenic | RGMA | U |
| 1705 | chr4 | 141146610 | 141146985 | Pancreatic Ductal cells | 0.19 | 0.91 | Intergenic | SCOC | U |
| 1706 | chr10 | 134471323 | 134471443 | Pancreatic Ductal cells | 0.13 | 0.84 | intron | INPP5A | U |
| 1707 | chr2 | 106985196 | 106985344 | Pancreatic Ductal cells | 0.16 | 0.87 | Intergenic | PLGLA | U |
| 1708 | chr2 | 150946052 | 150946328 | Pancreatic Ductal cells | 0.19 | 0.9 | Intergenic | RND3 | U |
| 1709 | chr4 | 77922281 | 7792512 | Pancreatic Ductal cells | 0.19 | 0.9 | intron | AFAP1 | U |
| 1710 | chr1 | 179265091 | 179265384 | Pancreatic Ductal cells | 0.18 | 0.89 | intron | SOAT1 | U |
| 1711 | chr18 | 44636607 | 44637031 | Pancreatic Ductal cells | 0.19 | 0.9 | intron | HDHD2 | U |
| 1712 | chr7 | 47632797 | 47633186 | Pancreatic Ductal cells | 0.11 | 0.81 | Intergenic | TNS3 | U |
| 1713 | chr15 | 36262769 | 36263172 | Pancreatic Ductal cells | 0.18 | 0.88 | Intergenic | MIR4510 | U |
| 1714 | chr1 | 203118091 | 203118226 | Pancreatic Ductal cells | 0.23 | 0.89 | intron | ADORA1 | U |
| 1715 | chr4 | 183210489 | 183210633 | Pancreatic Ductal cells | 0.19 | 0.88 | Intergenic | TENM3 | U |
| 1716 | chr4 | 721555383 | 72155868 | Pancreatic Ductal cells | 0.19 | 0.88 | intron | SLC4A4 | U |
| 1717 | chr4 | 72083022 | 72083235 | Pancreatic Ductal cells | 0.19 | 0.87 | intron | SLC4A4 | U |
| 1718 | chr8 | 29128535 | 29128781 | Pancreatic Ductal cells | 0.16 | 0.84 | Intergenic | KIF138 | U |
| 1719 | chr11 | 94916612 | 94916999 | Pancreatic Ductal cells | 0.24 | 0.92 | intron | SESN3 | U |
| 1720 | chr16 | 40383991 | 4038783 | Pancreatic Ductal cells | 0.2 | 0.87 | intron | ADCY9 | U |
| 1721 | chr10 | 126205456 | 126205501 | Pancreatic Ductal cells | 0.24 | 0.9 | intron | LHPP | U |
| 1722 | chr2 | 10496564 | 10496712 | Pancreatic Ductal cells | 0.22 | 0.88 | intron | HPCAL1 | U |
| 1723 | chr1 | 15187234 | 15187449 | Pancreatic Ductal cells | 0.18 | 0.84 | intron | KAZN | U |
| 1724 | chr16 | 56960148 | 56960377 | Pancreatic Ductal cells | 0.26 | 0.92 | Intergenic | HERPUD1 | U |
| 1725 | chr4 | 72135834 | 72136097 | Pancreatic Ductal cells | 0.26 | 0.92 | intron | SLC4A4 | U |
| 1726 | chr3 | 75378262 | 75378547 | Pancreatic Ductal cells | 0.21 | 0.87 | Intergenic | FAM86DP | U |
| 1727 | chr16 | 15984209 | 15984495 | Pancreatic Ductal cells | 0.19 | 0.84 | Intergenic | FOPNL | U |
| 1728 | chr8 | 25215773 | 25215892 | Pancreatic Ductal cells | 0.23 | 0.87 | intron | DOCK5 | U |
| 1729 | chr19 | 46181809 | 46182044 | Pancreatic Ductal cells | 0.24 | 0.88 | intron | GIPR | U |
| 1730 | chr4 | 72189693 | 72189957 | Pancreatic Ductal cells | 0.28 | 0.92 | intron | SLC4A4 | U |
| 1731 | chr1 | 2954308 | 2954394 | Pancreatic Ductal cells | 0.19 | 0.82 | Intergenic | ACTRT2 | U |
| 1732 | chr15 | 27818541 | 27818831 | Pancreatic Ductal cells | 0.24 | 0.87 | Intergenic | OCA2 | U |
| 1733 | chr7 | 912408773 | 91241031 | Pancreatic Ductal cells | 0.21 | 0.83 | Intergenic | MTERF | U |
| 1734 | chr5 | 37394810 | 37395283 | Pancreatic Ductal cells | 0.27 | 0.89 | intron | WDR70 | U |
| 1735 | chr20 | 56577840 | 56577972 | Pancreatic Ductal cells | 0.26 | 0.86 | Intergenic | MIR4532 | U |
| 1736 | chr21 | 40983093 | 40983371 | Pancreatic Ductal cells | 0.25 | 0.85 | intron | C21orf88 | U |
| 1737 | chr2 | 104956173 | 10496039 | Pancreatic Ductal cells | 0.31 | 0.9 | intron | HPCAL1 | U |
| 1738 | chr1 | 30153356 | 30153552 | Pancreatic Ductal cells | 0.32 | 0.9 | Intergenic | PTPRU | U |
| 1739 | chr7 | 68796860 | 68797241 | Pancreatic Ductal cells | 0.3 | 0.87 | Intergenic | AUTS2 | U |
| 1740 | chr14 | 100695022 | 100695326 | Pancreatic Ductal cells | 0.33 | 0.88 | Intergenic | YY1 | U |
| 1741 | chr1 | 41325000 | 41325363 | Pancreatic Ductal cells | 0.33 | 0.86 | Intergenic | CITED4 | U |
| 1742 | chr7 | 26492211 | 26492636 | Pancreatic Ductal cells | 0.38 | 0.9 | intron | LOC441204 | U |
| 1743 | chr4 | 20354428 | 20354667 | Pancreatic Ductal cells | 0.06 | 0.88 | intron | SLIT2 | U |
| 1744 | chr2 | 85477590 | 85477670 | Pancreatic Ductal cells | 0.12 | 0.9 | intron | TCF7L1 | U |
| 1745 | chr1 | 3196489 | 3196578 | Pancreatic Ductal cells | 0.04 | 0.81 | intron | PROM16 | U |
| 1746 | chr17 | 40528582 | 40528819 | Pancreatic Ductal cells | 0.15 | 0.9 | intron | STAT3 | U |
| 1747 | chr1 | 158169496 | 158169624 | Pancreatic Ductal cells | 0.29 | 0.91 | Intergenic | CD1D | U |
| 1748 | chr16 | 72958470 | 72958530 | Pancreatic Ductal cells | 0.03 | 0.92 | intron | ZFHX3 | U |
| 1749 | chr1 | 46949367 | 46949429 | Pancreatic Ductal cells | 0.04 | 0.85 | Intergenic | DMBX1 | U |
| 1750 | chr1 | 46949440 | 46949594 | Pancreatic Ductal cells | 0.19 | 0.92 | Intergenic | DMBX1 | U |
| 1751 | chr22 | 35966643 | 35967003 | Pancreatic Ductal cells | 0.18 | 0.88 | Intergenic | RASD2 | U |
| 1752 | chr1 | 180223424 | 180223479 | Pancreatic Ductal cells | 0.02 | 0.86 | intron | LHX4 | U |
| 1753 | chr6 | 14183709 | 14183951 | Pancreatic Ductal cells | 0.07 | 0.88 | Intergenic | CD83 | U |
| 1754 | chr14 | 100198943 | 100198981 | Pancreatic Ductal cells | 0.13 | 0.9 | Intergenic | CYP46A1 | U |
| 1755 | chr15 | 61012461 | 61012715 | Pancreatic Ductal cells | 0.17 | 0.92 | intron | RORA | U |
| 1756 | chr20 | 21291421 | 21291528 | Pancreatic Ductal cells | 0.19 | 0.92 | intron | XRN2 | U |
| 1757 | chr14 | 77481139 | 77481302 | Pancreatic Ductal cells | 0.18 | 0.91 | Intergenic | IRF2BPL | U |
| 1758 | chr9 | 40537857 | 40538036 | Pancreatic Ductal cells | 0.04 | 0.77 | Intergenic | SPATA31A3 | U |
| 1759 | chr9 | 86999839 | 87000244 | Pancreatic Ductal cells | 0.15 | 0.86 | Intergenic | SLC28A3 | U |
| 1760 | chr21 | 41040361 | 41040781 | Pancreatic Ductal cells | 0.17 | 0.87 | Intergenic | B3GALTS | U |
| 1761 | chr9 | 89131109 | 89131576 | Pancreatic Ductal cells | 0.15 | 0.85 | Intergenic | ZCCHC6 | U |
| 1762 | chr20 | 25189013 | 25189449 | Pancreatic Ductal cells | 0.22 | 0.9 | intron | ENTPD6 | U |
| 1763 | chr6 | 17929475 | 17929725 | Pancreatic Ductal cells | 0.2 | 0.87 | intron | KIF134 | U |
| 1764 | chr20 | 30957274 | 30957769 | Pancreatic Ductal cells | 0.27 | 0.88 | intron | ASXL1 | U |
| 1765 | chr14 | 65021983 | 65022099 | Pancreatic Ductal cells | 0.33 | 0.91 | intron | PPP1R36 | U |
| 1766 | chr7 | 48305992 | 48306449 | Pancreatic Ductal cells | 0.32 | 0.9 | intron | B4GALT5 | U |
| 1767 | chr7 | 139656224 | 139656474 | Pancreatic Ductal cells | 0.32 | 0.85 | intron | BXAS1 | U |
| 1768 | chr7 | 39125198 | 39125562 | Pancreatic Ductal cells | 0.86 | 0.16 | intron | POU6F2 | M |
| 1769 | chr13 | 28553792 | 28553833 | Pancreatic Ductal cells | 0.94 | 0.25 | intron | PRHOXNB | M |
| 1770 | chr13 | 285538421 | 28554244 | Pancreatic Ductal cells | 0.77 | 0.09 | intron | PRHOXNB | M |
| 1771 | chr19 | 48232782 | 48233027 | Pancreatic Ductal cells | 0.76 | 0.1 | intron | EHD2 | M |
| 1772 | chr10 | 73982876 | 73982966 | Pancreatic Ductal cells | 0.74 | 0.13 | intron | ANAPC16 | M |
| 1773 | chr6 | 91319986 | 91320098 | Pancreatic Ductal cells | 0.71 | 0.17 | Intergenic | MAP3K7 | M |
| 1774 | chr8 | 142215723 | 142215838 | Pancreatic Ductal cells | 0.65 | 0.11 | Intergenic | SLC45A4 | M |
| 1775 | chr2 | 235044324 | 235044527 | Pancreatic Ductal cells | 0.59 | 0.05 | Intergenic | SPP2 | M |
| 1776 | chr2 | 110519027 | 110519135 | Pancreatic Ductal cells | 0.69 | 0.2 | Intergenic | RGPD5 | M |
| 1777 | chr2 | 199174486 | 199174717 | Pancreatic Ductal cells | 0.55 | 0.06 | Intergenic | PLCL1 | M |
| 1778 | chr1 | 116961613 | 116961745 | Pancreatic Ductal cells | 0.56 | 0.09 | promoter-TSS | ATP1A1OS | M |
| 1779 | chr10 | 102107626 | 102107781 | Pancreatic Ductal cells | 0.55 | 0.1 | intron | SCD | M |
| 1780 | chr1 | 29805277 | 29805370 | Pancreatic Ductal cells | 0.91 | 0.18 | Intergenic | PTPRU | M |
| 1781 | chr11 | 94883896 | 94884066 | Pancreatic Ductal cells | 0.79 | 0.13 | Intergenic | ENDOD1 | M |
| 1782 | chr12 | 106978811 | 106979230 | Pancreatic Ductal cells | 0.7 | 0.1 | intron | RFX4 | M |
| 1783 | chr14 | 59103700 | 59103963 | Pancreatic Ductal cells | 0.75 | 0.17 | promoter-TSS | DACT1 | M |
| 1784 | chr5 | 115297291 | 115297621 | Pancreatic Ductal cells | 0.64 | 0.07 | promoter-TSS | AOPEP | M |
| 1785 | chr5 | 115297174 | 115297280 | Pancreatic Ductal cells | 0.66 | 0.1 | promoter-TSS | AQPEP | M |
| 1786 | chr5 | 115298920 | 115299089 | Pancreatic Ductal cells | 0.7 | 0.15 | exon | AQPEP | M |
| 1787 | chr6 | 26172931 | 26173215 | Pancreatic Ductal cells | 0.62 | 0.08 | TTS, Intergenic | HIST1H2BD, HIST1H2 [symbol] | M |
| 1788 | chr2 | 112789506 | 112789693 | Pancreatic Ductal cells | 0.64 | 0.12 | Intergenic | TMEM878 | M |
| 1789 | chr11 | 94884119 | 94884443 | Pancreatic Ductal cells | 0.56 | 0.05 | Intergenic | ENDOD1 | M |
| 1790 | chr13 | 28554355 | 28554449 | Pancreatic Ductal cells | 0.54 | 0.09 | intron | PRHOXNB | M |
| 1791 | chr21 | 46143588 | 46143656 | Pancreatic Ductal cells | 0.54 | 0.12 | Intergenic | TSPEAR | M |
| 1792 | chr21 | 46130516 | 46130757 | Pancreatic Ductal cells | 0.54 | 0.13 | intron | TSPEAR | M |
| 1793 | chr4 | 183272479 | 183272543 | Endometrium Epithelium | 0.1 | 0.9 | intron | TENM3 | U |
| 1794 | chr1 | 88737996 | 88738097 | Endometrium Epithelium | 0.1 | 0.9 | Intergenic | PKN2 | U |
| 1795 | chr11 | 61264069 | 61264257 | Endometrium Epithelium | 0.08 | 0.88 | Intergenic | MIR4488 | U |
| 1796 | chr10 | 43819264 | 43819432 | Endometrium Epithelium | 0.14 | 0.91 | Intergenic | FXYD4 | U |
| 1797 | chr8 | 8094386 | 8094714 | Endometrium Epithelium | 0.12 | 0.88 | intron | FAM8683P | U |
| 1798 | chr1 | 151940571 | 15194102 | Endometrium Epithelium | 0.16 | 0.91 | intron | KAZN | U |
| 1799 | chr13 | 1065973731 | 106597474 | Endometrium Epithelium | 0.18 | 0.93 | Intergenic | LINC00343 | U |
| 1800 | chr19 | 1340629 | 1340759 | Endometrium Epithelium | 0.1 | 0.85 | Intergenic | MUM1 | U |
| 1801 | chr11 | 71506971 | 71507101 | Endometrium Epithelium | 0.13 | 0.87 | TTS, exon | ALG1L9P, FAM86C1 | U |
| 1802 | chr15 | 75839798 | 75840092 | Endometrium Epithelium | 0.17 | 0.9 | intron | PTPN9 | U |
| 1803 | chr21 | 434957931 | 43495870 | Endometrium Epithelium | 0.18 | 0.9 | intron | UMODL1 | U |
| 1804 | chr3 | 319837233 | 31984006 | Endometrium Epithelium | 0.18 | 0.9 | intron | OSBPL10 | U |
| 1805 | chr2 | 349782571 | 34978307 | Endometrium Epithelium | 0.2 | 0.91 | Intergenic | MYADML | U |
| 1806 | chr14 | 105280371 | 105280545 | Endometrium Epithelium | 0.07 | 0.78 | Intergenic | LINC00638 | U |
| 1807 | chr5 | 172371226 | 172371425 | Endometrium Epithelium | 0.17 | 0.88 | intron | ERGIC1 | U |
| 1808 | chr5 | 125589059 | 125589358 | Endometrium Epithelium | 0.19 | 0.9 | Intergenic | HDDC2 | U |
| 1809 | chr2 | 188371809 | 188372138 | Endometrium Epithelium | 0.19 | 0.9 | intron | TFPI | U |
| 1810 | chr9 | 1320485161 | 132048588 | Endometrium Epithelium | 0.23 | 0.93 | Intergenic | C9orf106 | U |
| 1811 | chr16 | 1194503 | 1194648 | Endometrium Epithelium | 0.16 | 0.86 | Intergenic | CACNA1H | U |
| 1812 | chr16 | 803569 | 803729 | Endometrium Epithelium | 0.11 | 0.81 | Intergenic | MSLN | U |
| 1813 | chr15 | 380171681 | 38017344 | Endometrium Epithelium | 0.16 | 0.86 | Intergenic | TMCO5A | U |
| 1814 | chr19 | 10314970 | 10315256 | Endometrium Epithelium | 0.17 | 0.87 | Intergenic | DNMT1 | U |
| 1815 | chr21 | 37752927 | 37752973 | Endometrium Epithelium | 0.25 | 0.94 | Intergenic | CHAF1B | U |
| 1816 | chr16 | 83914357 | 83914480 | Endometrium Epithelium | 0.17 | 0.86 | Intergenic | MLYCD | U |
| 1817 | chr19 | 56758878 | 56759186 | Endometrium Epithelium | 0.19 | 0.88 | Intergenic | ZSCAN5A | U |
| 1818 | chr19 | 136924481 | 13692513 | Endometrium Epithelium | 0.19 | 0.87 | Intergenic | CACNA1A | U |
| 1819 | chr17 | 803486281 | 80348805 | Endometrium Epithelium | 0.25 | 0.93 | exon | OGFOD3 | U |
| 1820 | chr1 | 16446059 | 16446276 | Endometrium Epithelium | 0.14 | 0.82 | Intergenic | EPHA2 | U |
| 1821 | chr17 | 27408345 | 27408672 | Endometrium Epithelium | 0.23 | 0.91 | intron | MYO18A | U |
| 1822 | chr6 | 45643631 | 45643774 | Endometrium Epithelium | 0.19 | 0.86 | Intergenic | RUNX2 | U |
| 1823 | chr1 | 2141909863 | 214191109 | Endometrium Epithelium | 0.23 | 0.89 | intron | PROX1 | U |
| 1824 | chr11 | 109709660 | 109709824 | Endometrium Epithelium | 0.18 | 0.84 | Intergenic | ZC3H12C | U |
| 1825 | chr6 | 34432216 | 34432388 | Endometrium Epithelium | 0.2 | 0.86 | Intergenic | PACSIN1 | U |
| 1826 | chr7 | 261760431 | 26176234 | Endometrium Epithelium | 0.22 | 0.88 | Intergenic | NFE2L3 | U |
| 1827 | chr2 | 116301176 | 116301488 | Endometrium Epithelium | 0.19 | 0.85 | intron | DPP10 | U |
| 1828 | chr2 | 61740650 | 61740740 | Endometrium Epithelium | 0.26 | 0.91 | intron | XPO1 | U |
| 1829 | chr4 | 1832727011 | 183272841 | Endometrium Epithelium | 0.15 | 0.8 | intron | TENM3 | U |
| 1830 | chr8 | 125977953 | 125978122 | Endometrium Epithelium | 0.13 | 0.78 | Intergenic | ZNF572 | U |
| 1831 | chr11 | 36501472 | 36501671 | Endometrium Epithelium | 0.16 | 0.81 | Intergenic | TRAF6 | U |
| 1832 | chr11 | 3435238 | 3435496 | Endometrium Epithelium | 0.23 | 0.88 | Intergenic | OR7E12P | U |
| 1833 | chr7 | 1763593 | 1763701 | Endometrium Epithelium | 0.16 | 0.8 | intron | ELFN1 | U |
| 1834 | chr4 | 182996382 | 182996573 | Endometrium Epithelium | 0.24 | 0.88 | Intergenic | MGC45800 | U |
| 1835 | chr4 | 181648322 | 181648635 | Endometrium Epithelium | 0.18 | 0.82 | Intergenic | LINC00290 | U |
| 1836 | chr8 | 28183689 | 28184008 | Endometrium Epithelium | 0.23 | 0.87 | intron | PNOC | U |
| 1837 | chr10 | 87796575 | 87796909 | Endometrium Epithelium | 0.19 | 0.83 | intron | GRID1 | U |
| 1838 | chr5 | 793325603 | 79332751 | Endometrium Epithelium | 0.2 | 0.83 | intron | THBS4 | U |
| 1839 | chr22 | 45903659 | 45903852 | Endometrium Epithelium | 0.22 | 0.85 | intron | FBLN1 | U |
| 1840 | chr4 | 82161341 | 8216352 | Endometrium Epithelium | 0.26 | 0.89 | exon | SH3TC1 | U |
| 1841 | chr6 | 1510760333 | 151076269 | Endometrium Epithelium | 0.24 | 0.87 | intron | PLEKHG1 | U |
| 1842 | chr1 | 222960114 | 222960445 | Endometrium Epithelium | 0.2 | 0.83 | Intergenic | DISP1 | U |
| 1843 | chr9 | 1381113253 | 138111391 | Endometrium Epithelium | 0.18 | 0.8 | Intergenic | LOC401557 | U |
| 1844 | chr18 | 8588101 | 858922 | Endometrium Epithelium | 0.21 | 0.83 | Intergenic | ADCYAP1 | U |
| 1845 | chr6 | 159279614 | 159279760 | Endometrium Epithelium | 0.22 | 0.84 | promoter-TSS, Interg [symbol] | OSTCP1, OSTCP1 | U |
| 1846 | chr7 | 6974962 | 6975158 | Endometrium Epithelium | 0.24 | 0.86 | Intergenic | CCZ1B | U |
| 1847 | chr17 | 17492886 | 17493190 | Endometrium Epithelium | 0.24 | 0.85 | intron | PEMT | U |
| 1848 | chr11 | 40413021 | 40413462 | Endometrium Epithelium | 0.23 | 0.84 | intron | LRRC4C | U |
| 1849 | chr3 | 2847616 | 2847687 | Endometrium Epithelium | 0.23 | 0.83 | intron | CNTN4 | U |
| 1850 | chr12 | 97092440 | 97092685 | Endometrium Epithelium | 0.23 | 0.82 | Intergenic | NEDD1 | U |
| 1851 | chr12 | 78605315 | 78605603 | Endometrium Epithelium | 0.27 | 0.86 | exon | NAV3 | U |
| 1852 | chr10 | 114972951 | 114973394 | Endometrium Epithelium | 0.26 | 0.85 | Intergenic | TCF7L2 | U |
| 1853 | chr10 | 125063726 | 125064172 | Endometrium Epithelium | 0.21 | 0.8 | Intergenic | BUB3 | U |
| 1854 | chr14 | 996611183 | 99661175 | Endometrium Epithelium | 0.28 | 0.86 | intron | BCL11B | U |
| 1855 | chr11 | 1264353711 | 126435449 | Endometrium Epithelium | 0.27 | 0.85 | intron | KIRREL3 | U |
| 1856 | chr20 | 57392983 | 57393278 | Endometrium Epithelium | 0.27 | 0.85 | promoter-TSS | MIR296 | U |
| 1857 | chr2 | 18429524 | 18429969 | Endometrium Epithelium | 0.31 | 0.89 | Intergenic | RDH14 | U |
| 1858 | chr5 | 178928923 | 178929074 | Endometrium Epithelium | 0.24 | 0.79 | Intergenic | RUFY1 | U |
| 1859 | chr9 | 128745913 | 128746123 | Endometrium Epithelium | 0.24 | 0.78 | Intergenic | PBX3 | U |
| 1860 | chr7 | 1568827223 | 156883060 | Endometrium Epithelium | 0.26 | 0.8 | Intergenic | UBE3C | U |
| 1861 | chr18 | 764344433 | 75434499 | Endometrium Epithelium | 0.31 | 0.84 | Intergenic | SALL3 | U |
| 1862 | chr1 | 22915453 | 22915513 | Endometrium Epithelium | 0.28 | 0.81 | intron, exon | EPHA8, EPHA8 | U |
| 1863 | chr20 | 1753923 | 1754095 | Endometrium Epithelium | 0.3 | 0.81 | intron, TTS | LOC100289473, LOC1 [symbol] | U |
| 1864 | chr7 | 151379729 | 151379984 | Endometrium Epithelium | 0.36 | 0.82 | intron | PRKAG2 | U |
| 1865 | chr6 | 152133802 | 152134204 | Endometrium Epithelium | 0.02 | 0.83 | intron | ESR1 | U |
| 1866 | chr6 | 152132806 | 152133188 | Endometrium Epithelium | 0.01 | 0.81 | intron | ESR1 | U |
| 1867 | chr6 | 152001630 | 152002105 | Endometrium Epithelium | 0.19 | 0.92 | Intergenic | ESR1 | U |
| 1868 | chr3 | 191661320 | 191661582 | Endometrium Epithelium | 0.24 | 0.91 | Intergenic | FGF12 | U |
| 1869 | chr16 | 83758968 | 83759181 | Endometrium Epithelium | 0.22 | 0.83 | intron | CDH13 | U |
| 1870 | chr2 | 69013996 | 69014333 | Endometrium Epithelium | 0.27 | 0.85 | intron | ARHGAP25 | U |
| 1871 | chr16 | 87716530 | 87716660 | Endometrium Epithelium | 0.2 | 0.77 | intron | JPH3 | U |
| 1872 | chrX | 44642544 | 44642681 | Endometrium Epithelium | 0.34 | 0.82 | Intergenic | DUSP21 | U |
| 1873 | chr1 | 233085132 | 233085445 | Endometrium Epithelium | 0.09 | 0.91 | promoter-TSS, Interg [symbol] | NTPCR, NTPCR | U |
| 1874 | chr16 | 89770185 | 89770271 | Endometrium Epithelium | 0.1 | 0.88 | Intergenic | SPATA2L | U |
| 1875 | chr11 | 133704360 | 133704489 | Endometrium Epithelium | 0.12 | 0.88 | Intergenic | SPATA19 | U |
| 1876 | chr7 | 42237271 | 42237445 | Endometrium Epithelium | 0.14 | 0.9 | intron | GLI3 | U |
| 1877 | chr4 | 95461312 | 95461430 | Endometrium Epithelium | 0.2 | 0.94 | intron | PDLIM5 | U |
| 1878 | chr12 | 60384612 | 60384730 | Endometrium Epithelium | 0.15 | 0.89 | Intergenic | SLC16A7 | U |
| 1879 | chr13 | 114812334 | 114812657 | Endometrium Epithelium | 0.12 | 0.84 | intron | RASA3 | U |
| 1880 | chr12 | 94235572 | 94235768 | Endometrium Epithelium | 0.23 | 0.94 | intron | CRADD | U |
| 1881 | chr5 | 171427293 | 171427624 | Endometrium Epithelium | 0.21 | 0.91 | intron | FBXW11 | U |
| 1882 | chr9 | 133366689 | 133366753 | Endometrium Epithelium | 0.21 | 0.88 | intron | ASS1 | U |
| 1883 | chr22 | 33711176 | 33711425 | Endometrium Epithelium | 0.21 | 0.87 | intron | LARGE | U |
| 1884 | chr9 | 71192444 | 71192751 | Endometrium Epithelium | 0.15 | 0.81 | Intergenic | TMEM252 | U |
| 1885 | chr10 | 134795905 | 134796062 | Endometrium Epithelium | 0.2 | 0.84 | Intergenic | LOC399829 | U |
| 1886 | chr9 | 115630973 | 115631205 | Endometrium Epithelium | 0.27 | 0.88 | exon | SNX30 | U |
| 1887 | chr6 | 13880933 | 13881207 | Endometrium Epithelium | 0.3 | 0.9 | Intergenic | RNF182 | U |
| 1888 | chr22 | 42108873 | 42109109 | Endometrium Epithelium | 0.28 | 0.87 | intron | MEI1 | U |
| 1889 | chr19 | 1334184 | 1334262 | Endometrium Epithelium | 0.24 | 0.82 | Intergenic | MUM1 | U |
| 1890 | chr10 | 134610571 | 134610862 | Endometrium Epithelium | 0.28 | 0.86 | Intergenic | NKX6-2 | U |
| 1891 | chr9 | 27490795 | 27491117 | Endometrium Epithelium | 0.28 | 0.83 | intron | MOB3B | U |
| 1892 | chr20 | 41976096 | 41976492 | Endometrium Epithelium | 0.24 | 0.78 | Intergenic | SRSF6 | U |
| 1893 | chr1 | 208132328 | 208132348 | Endometrium Epithelium | 0.81 | 0.25 | Intergenic | CD34 | M |
| 1894 | chr2 | 110372837 | 110373003 | Endometrium Epithelium | 0.72 | 0.17 | exon | SOWAHC | M |
| 1895 | chr1 | 196866430 | 196866530 | Endometrium Epithelium | 0.72 | 0.21 | intron | CFHR4 | M |
| 1896 | chrX | 17392781 | 17392927 | Endometrium Epithelium | 0.68 | 0.18 | promoter-TSS | NHS | M |
| 1897 | chr3 | 186490115 | 186490280 | Endometrium Epithelium | 0.63 | 0.13 | Intergenic | EIF4A2 | M |
| 1898 | chrX | 99663193 | 99663331 | Endometrium Epithelium | 0.72 | 0.24 | exon | PCDH19 | M |
| 1899 | chr3 | 157260711 | 157260870 | Endometrium Epithelium | 0.58 | 0.12 | promoter-TSS | C3orf55 | M |
| 1900 | chr10 | 62761559 | 62761642 | Endometrium Epithelium | 0.72 | 0.27 | promoter-TSS | RHOBTB1 | M |
| 1901 | chr3 | 71802355 | 71802467 | Endometrium Epithelium | 0.6 | 0.18 | promoter-TSS | GPR27 | M |
| 1902 | chrX | 117958654 | 117958794 | Endometrium Epithelium | 0.74 | 0.32 | intron | ZCCHC12 | M |
| 1903 | chr19 | 49112138 | 49112279 | Endometrium Epithelium | 0.63 | 0.28 | intron | FAM83 [symbol] | M |
| 1904 | chr8 | 16801894 | 16801959 | Endometrium Epithelium | 0.61 | 0.27 | Intergenic | FGF20 | M |
| 1905 | chr7 | 94023465 | 94023568 | Endometrium Epithelium | 0.54 | 0.22 | promoter-TSS | COL1A2 | M |
| 1906 | chr16 | 51168402 | 51168685 | Endometrium Epithelium | 0.86 | 0.14 | Intergenic | SALL1 | M |
| 1907 | chr16 | 51168132 | 51168400 | Endometrium Epithelium | 0.82 | 0.13 | Intergenic | SALL1 | M |
| 1908 | chr4 | 106066999 | 106067274 | Endometrium Epithelium | 0.69 | 0.05 | promoter-TSS, intro [symbol] | TET2, TET2 | M |
| 1909 | chr5 | 138923135 | 138923219 | Endometrium Epithelium | 0.73 | 0.1 | Intergenic | UBE2D2 | M |
| 1910 | chr12 | 1306478901 | 130648088 | Endometrium Epithelium | 0.74 | 0.13 | exon | FZD10 | M |
| 1911 | chr20 | 9489269 | 9489565 | Endometrium Epithelium | 0.77 | 0.16 | Intergenic | LAMP5 | M |
| 1912 | chr14 | 105512062 | 105512417 | Endometrium Epithelium | 0.63 | 0.03 | Intergenic | GPR132 | M |
| 1913 | chr20 | 9488976 | 9489231 | Endometrium Epithelium | 0.68 | 0.09 | Intergenic | LAMP5 | M |
| 1914 | chr10 | 8097552 | 8097796 | Endometrium Epithelium | 0.64 | 0.08 | exon | GATA3 | M |
| 1915 | chr2 | 54798129 | 54798348 | Endometrium Epithelium | 0.63 | 0.1 | intron | SPTBN1 | M |
| 1916 | chr10 | 1024731183 | 102473300 | Endometrium Epithelium | 0.56 | 0.15 | Intergenic | PAX2 | M |
| 1917 | chrX | 99663395 | 99663546 | Endometrium Epithelium | 0.6 | 0.24 | exon | PCDH19 | M |
| 1918 | chr1 | 172579244 | 172579386 | Fallopian Epithelium | 0.02 | 0.91 | exon | SUCO | U |
| 1919 | chr17 | 63004755 | 63004919 | Fallopian Epithelium | 0.05 | 0.94 | Intergenic, TTS | AMZ2P1, GNA13 | U |
| 1920 | chr4 | 22604364 | 22604525 | Fallopian Epithelium | 0.04 | 0.92 | Intergenic | GPR125 | U |
| 1921 | chr17 | 12902144 | 12902625 | Fallopian Epithelium | 0.06 | 0.94 | intron | ELAC2 | U |
| 1922 | chr6 | 167457159 | 167457484 | Fallopian Epithelium | 0.03 | 0.9 | Intergenic | FGFR1OP | U |
| 1923 | chr1 | 111967898 | 111968105 | Fallopian Epithelium | 0.03 | 0.89 | intron | OVGP1 | U |
| 1924 | chr8 | 17350396 | 17350622 | Fallopian Epithelium | 0.02 | 0.88 | Intergenic | SLC7A2 | U |
| 1925 | chr10 | 88403481 | 88403562 | Fallopian Epithelium | 0.01 | 0.86 | Intergenic | OPN4 | U |
| 1926 | chr4 | 22603266 | 22603729 | Fallopian Epithelium | 0.09 | 0.93 | Intergenic | GPR125 | U |
| 1927 | chr8 | 145249327 | 145249613 | Fallopian Epithelium | 0.08 | 0.91 | intron | MROH1 | U |
| 1928 | chr21 | 16915668 | 16915942 | Fallopian Epithelium | 0.04 | 0.85 | Intergenic | USP25 | U |
| 1929 | chr1 | 111985459 | 111985650 | Fallopian Epithelium | 0.12 | 0.92 | intron | WDR77 | U |
| 1930 | chr2 | 148404745 | 148405073 | Fallopian Epithelium | 0.06 | 0.85 | Intergenic | ACVR2A | U |
| 1931 | chr3 | 178101164 | 178101550 | Fallopian Epithelium | 0.08 | 0.87 | Intergenic | KCNMB2-IT1 | U |
| 1932 | chr13 | 20513194 | 20513415 | Fallopian Epithelium | 0.12 | 0.9 | Intergenic | ZMYM2 | U |
| 1933 | chr8 | 64032053 | 64032428 | Fallopian Epithelium | 0.13 | 0.91 | Intergenic | TTPA | U |
| 1934 | chr3 | 9312551 | 9312788 | Fallopian Epithelium | 0.11 | 0.87 | Intergenic | SRGAP3 | U |
| 1935 | chr16 | 83997324 | 83997562 | Fallopian Epithelium | 0.1 | 0.86 | intron | OSGIN1 | U |
| 1936 | chr7 | 123510738 | 123510841 | Fallopian Epithelium | 0.15 | 0.9 | intron | HYAL4 | U |
| 1937 | chr2 | 173721702 | 173722198 | Fallopian Epithelium | 0.25 | 0.93 | intron | RAPGEF4 | U |
| 1938 | chr2 | 101548181 | 101548341 | Fallopian Epithelium | 0.05 | 0.94 | intron | NPAS2 | U |
| 1939 | chr3 | 195237882 | 195238137 | Fallopian Epithelium | 0.04 | 0.93 | Intergenic | MIR5692C1 | U |
| 1940 | chr10 | 27488102 | 27488189 | Fallopian Epithelium | 0.05 | 0.92 | intron | ACBD5 | U |
| 1941 | chr2 | 5640117 | 5640288 | Fallopian Epithelium | 0.01 | 0.87 | Intergenic | SOX11 | U |
| 1942 | chr6 | 155328330 | 155328620 | Fallopian Epithelium | 0.04 | 0.89 | Intergenic | TIAM2 | U |
| 1943 | chr5 | 61694414 | 61694897 | Fallopian Epithelium | 0.04 | 0.89 | exon | DIMT1 | U |
| 1944 | chr17 | 60790869 | 60790952 | Fallopian Epithelium | 0.04 | 0.88 | intron | MARCH10 | U |
| 1945 | chr3 | 185745309 | 185745404 | Fallopian Epithelium | 0.08 | 0.92 | Intergenic | LOC344887 | U |
| 1946 | chr9 | 135486165 | 135486346 | Fallopian Epithelium | 0.09 | 0.92 | intron | DDX31 | U |
| 1947 | chr11 | 71923939 | 71924198 | Fallopian Epithelium | 0.02 | 0.85 | Intergenic | FOLR2 | U |
| 1948 | chr4 | 57633113 | 57633291 | Fallopian Epithelium | 0.07 | 0.89 | Intergenic | SPINK2 | U |
| 1949 | chr4 | 145268668 | 145268888 | Fallopian Epithelium | 0.01 | 0.83 | Intergenic | GYPA | U |
| 1950 | chr1 | 244476799 | 244477251 | Fallopian Epithelium | 0.07 | 0.89 | Intergenic | C1orf100 | U |
| 1951 | chr12 | 110801079 | 110801295 | Fallopian Epithelium | 0.1 | 0.91 | Intergenic | ANAPC7 | U |
| 1952 | chr2 | 1545713 | 1545942 | Fallopian Epithelium | 0.03 | 0.83 | intron | TPO | U |
| 1953 | chr7 | 29350848 | 29351118 | Fallopian Epithelium | 0.08 | 0.88 | intron | CHN2 | U |
| 1954 | chr5 | 176810230 | 176810566 | Fallopian Epithelium | 0.05 | 0.85 | Intergenic | SLC34A1 | U |
| 1955 | chr18 | 19717464 | 19717894 | Fallopian Epithelium | 0.08 | 0.88 | Intergenic | GATA6 | U |
| 1956 | chr11 | 97778616 | 97779053 | Fallopian Epithelium | 0.07 | 0.87 | Intergenic | JRKL-AS1 | U |
| 1957 | chr11 | 69013717 | 69013783 | Fallopian Epithelium | 0.06 | 0.85 | Intergenic | MYEOV | U |
| 1958 | chr8 | 24133807 | 24133996 | Fallopian Epithelium | 0.1 | 0.89 | Intergenic | ADAM28 | U |
| 1959 | chr10 | 14320514 | 14320703 | Fallopian Epithelium | 0.01 | 0.8 | intron | FRMD4A | U |
| 1960 | chr5 | 60822108 | 60822341 | Fallopian Epithelium | 0.13 | 0.92 | intron | ZSWIM6 | U |
| 1961 | chr1 | 94165060 | 94165443 | Fallopian Epithelium | 0.1 | 0.89 | intron | BCAR3 | U |
| 1962 | chr10 | 3709640 | 3709769 | Fallopian Epithelium | 0.1 | 0.88 | Intergenic | KLF6 | U |
| 1963 | chr17 | 75113353 | 75113518 | Fallopian Epithelium | 0.07 | 0.85 | intron | SEC14L1 | U |
| 1964 | chr2 | 236909064 | 236909368 | Fallopian Epithelium | 0.14 | 0.92 | intron | AGAP1 | U |
| 1965 | chr17 | 74807275 | 74807636 | Fallopian Epithelium | 0.08 | 0.86 | Intergenic | MGAT5B | U |
| 1966 | chr3 | 5450781 | 5450949 | Fallopian Epithelium | 0.08 | 0.85 | Intergenic | MIR4790 | U |
| 1967 | chr3 | 11289659 | 11289853 | Fallopian Epithelium | 0.12 | 0.89 | intron | HRH1 | U |
| 1968 | chr11 | 11092363 | 11092685 | Fallopian Epithelium | 0.09 | 0.86 | Intergenic | LOC729013 | U |
| 1969 | chr17 | 53549234 | 53549559 | Fallopian Epithelium | 0.11 | 0.88 | Intergenic | MMD | U |
| 1970 | chr19 | 29512635 | 29512788 | Fallopian Epithelium | 0.09 | 0.85 | Intergenic | LOC100505835 | U |
| 1971 | chr2 | 129666820 | 129667077 | Fallopian Epithelium | 0.07 | 0.83 | Intergenic | HS6ST1 | U |
| 1972 | chr5 | 173113621 | 173114000 | Fallopian Epithelium | 0.13 | 0.89 | Intergenic | BOD1 | U |
| 1973 | chr12 | 83788743 | 83789144 | Fallopian Epithelium | 0.09 | 0.85 | Intergenic | TMTC2 | U |
| 1974 | chr16 | 87511831 | 87512238 | Fallopian Epithelium | 0.16 | 0.92 | intron | ZCCHC14 | U |
| 1975 | chr1 | 111962901 | 111963329 | Fallopian Epithelium | 0.1 | 0.86 | intron | OVGP1 | U |
| 1976 | chr4 | 170641059 | 170641488 | Fallopian Epithelium | 0.1 | 0.86 | exon | CLCN3 | U |
| 1977 | chr1 | 111986202 | 111986679 | Fallopian Epithelium | 0.1 | 0.86 | intron | WDR77 | U |
| 1978 | chr8 | 25184019 | 25184117 | Fallopian Epithelium | 0.12 | 0.87 | intron | DOCK5 | U |
| 1979 | chr8 | 30008237 | 30008488 | Fallopian Epithelium | 0.07 | 0.82 | intron | MIR54802 | U |
| 1980 | chr5 | 53401718 | 53402149 | Fallopian Epithelium | 0.12 | 0.87 | intron | GCLC | U |
| 1981 | chr15 | 90586693 | 90587017 | Fallopian Epithelium | 0.15 | 0.89 | intron | ZNF710 | U |
| 1982 | chr6 | 162254595 | 162254688 | Fallopian Epithelium | 0.12 | 0.85 | intron | PARK2 | U |
| 1983 | chr2 | 15948155 | 15948337 | Fallopian Epithelium | 0.13 | 0.86 | Intergenic | MYCN | U |
| 1984 | chr11 | 100080833 | 100081069 | Fallopian Epithelium | 0.13 | 0.85 | intron | CNTN5 | U |
| 1985 | chr9 | 1255665833 | 125566829 | Fallopian Epithelium | 0.2 | 0.92 | Intergenic | OR1K1 | U |
| 1986 | chr21 | 43940842 | 43941201 | Fallopian Epithelium | 0.12 | 0.84 | intron | SLC37A1 | U |
| 1987 | chr18 | 76546040 | 76546267 | Fallopian Epithelium | 0.16 | 0.87 | Intergenic | SALL3 | U |
| 1988 | chr11 | 12233727 | 12234011 | Fallopian Epithelium | 0.2 | 0.91 | intron | MICAL2 | U |
| 1989 | chr1 | 22819009 | 22819482 | Fallopian Epithelium | 0.18 | 0.89 | intron | ZBTB40 | U |
| 1990 | chr3 | 11215119 | 11215615 | Fallopian Epithelium | 0.17 | 0.88 | intron | HRH1 | U |
| 1991 | chr5 | 15937562 | 15937626 | Fallopian Epithelium | 0.21 | 0.91 | exon | FBXL7 | U |
| 1992 | chr4 | 146298122 | 146298221 | Fallopian Epithelium | 0.13 | 0.83 | Intergenic | SMAD1 | U |
| 1993 | chr1 | 230916064 | 230916192 | Fallopian Epithelium | 0.17 | 0.87 | intron | CAPN9 | U |
| 1994 | chr3 | 1366902623 | 136690399 | Fallopian Epithelium | 0.24 | 0.94 | intron | IL20RB | U |
| 1995 | chr14 | 93342482 | 93342779 | Fallopian Epithelium | 0.12 | 0.82 | Intergenic | CHGA | U |
| 1996 | chr2 | 221081171 | 221081471 | Fallopian Epithelium | 0.19 | 0.89 | Intergenic | MIR4268 | U |
| 1997 | chr20 | 60537308 | 60537616 | Fallopian Epithelium | 0.13 | 0.82 | Intergenic | MIR1257 | U |
| 1998 | chr4 | 8476417 | 8476566 | Fallopian Epithelium | 0.16 | 0.84 | intron | TRMT44 | U |
| 1999 | chr8 | 37167163 | 37167406 | Fallopian Epithelium | 0.18 | 0.86 | Intergenic | ZNF703 | U |
| 2000 | chr2 | 113623217 | 113623649 | Fallopian Epithelium | 0.18 | 0.86 | Intergenic | IL1B | U |
| 2001 | chr14 | 95339286 | 95339521 | Fallopian Epithelium | 0.21 | 0.88 | Intergenic | GSC | U |
| 2002 | chr7 | 1429993201 | 142999770 | Fallopian Epithelium | 0.22 | 0.89 | intron | CASP2 | U |
| 2003 | chr1 | 6938724 | 6938836 | Fallopian Epithelium | 0.28 | 0.94 | intron | CAMTA1 | U |
| 2004 | chr3 | 72058519 | 72058673 | Fallopian Epithelium | 0.25 | 0.91 | Intergenic | LOC201617 | U |
| 2005 | chr17 | 62663832 | 62664044 | Fallopian Epithelium | 0.24 | 0.9 | Intergenic | SMURF2 | U |
| 2006 | chr4 | 79878229 | 79878503 | Fallopian Epithelium | 0.17 | 0.83 | Intergenic | LOC100505875 | U |
| 2007 | chr9 | 138356237 | 138356676 | Fallopian Epithelium | 0.27 | 0.92 | exon | LOC100506599 | U |
| 2008 | chr7 | 66397994 | 66398237 | Fallopian Epithelium | 0.25 | 0.89 | intron | TMEM248 | U |
| 2009 | chr8 | 77932187 | 77932525 | Fallopian Epithelium | 0.25 | 0.89 | Intergenic | PEX2 | U |
| 2010 | chr18 | 42862917 | 42863362 | Fallopian Epithelium | 0.27 | 0.91 | intron | SLC14A2 | U |
| 2011 | chr2 | 219787077 | 219787509 | Fallopian Epithelium | 0.19 | 0.82 | Intergenic | CDK5R2 | U |
| 2012 | chr5 | 112799248 | 112799700 | Fallopian Epithelium | 0.21 | 0.84 | intron | MCC | U |
| 2013 | chr5 | 65538308 | 65538600 | Fallopian Epithelium | 0.26 | 0.88 | Intergenic | SREKI | U |
| 2014 | chr21 | 35515274 | 35515581 | Fallopian Epithelium | 0.3 | 0.92 | TTS | MRPS6 | U |
| 2015 | chr2 | 173448055 | 173448390 | Fallopian Epithelium | 0.32 | 0.94 | intron | PDK1 | U |
| 2016 | chr13 | 37788409 | 37788774 | Fallopian Epithelium | 0.26 | 0.88 | Intergenic | CSNK1A1L | U |
| 2017 | chr4 | 17533070 | 17533270 | Fallopian Epithelium | 0.25 | 0.86 | Intergenic | CLRN2 | U |
| 2018 | chr3 | 130635214 | 130635456 | Fallopian Epithelium | 0.33 | 0.94 | intron | ATP2C1 | U |
| 2019 | chr17 | 66671291 | 66671602 | Fallopian Epithelium | 0.29 | 0.9 | Intergenic | FAM204 | U |
| 2020 | chr1 | 41973448 | 41973852 | Fallopian Epithelium | 0.31 | 0.91 | exon | HIVEP3 | U |
| 2021 | chr12 | 92588638 | 92588846 | Fallopian Epithelium | 0.35 | 0.92 | Intergenic | BTG1 | U |
| 2022 | chr12 | 8828813 | 8829136 | Fallopian Epithelium | 0.3 | 0.85 | Intergenic | MFAP5 | U |
| 2023 | chr10 | 1234487941 | 123449223 | Fallopian Epithelium | 0.08 | 0.8 | Intergenic | FGFR2 | U |
| 2024 | chr2 | 1932785453 | 193278786 | Fallopian Epithelium | 0.24 | 0.86 | Intergenic | TMEFF2 | U |
| 2025 | chr21 | 45483246 | 45483618 | Fallopian Epithelium | 0.04 | 0.94 | exon, intron | TRAPPC10 | U |
| 2026 | chr14 | 46854120 | 46854410 | Fallopian Epithelium | 0.08 | 0.9 | Intergenic | RPL10L | U |
| 2027 | chr14 | 51894318 | 51894732 | Fallopian Epithelium | 0.18 | 0.9 | Intergenic | LINC00640 | U |
| 2028 | chr5 | 15937192 | 15937404 | Fallopian Epithelium | 0.27 | 0.94 | lexor | FBXL7 | U |
| 2029 | chr2 | 206890514 | 206890842 | Fallopian Epithelium | 0.32 | 0.94 | intron | NOBOD | U |
| 2030 | chr14 | 66423698 | 66423803 | Fallopian Epithelium | 0.04 | 0.89 | Intergenic | LINC00238 | U |
| 2031 | chr14 | 75363159 | 75363611 | Fallopian Epithelium | 0.06 | 0.91 | intron | DLST | U |
| 2032 | chr12 | 51844686 | 51844830 | Fallopian Epithelium | 0.03 | 0.87 | exon | SLC4A8 | U |
| 2033 | chr9 | 78935216 | 78935535 | Fallopian Epithelium | 0.05 | 0.87 | intron | PCSK5 | U |
| 2034 | chr10 | 134593141 | 134593533 | Fallopian Epithelium | 0.12 | 0.94 | intron | INPP5A | U |
| 2035 | chr9 | 86409444 | 86409869 | Fallopian Epithelium | 0.08 | 0.89 | intron | GKAP1 | U |
| 2036 | chr14 | 532002001 | 53200644 | Fallopian Epithelium | 0.12 | 0.92 | intron | STYX | U |
| 2037 | chr7 | 155871852 | 155871961 | Fallopian Epithelium | 0.12 | 0.91 | Intergenic | SHH | U |
| 2038 | chr6 | 550533 | 550713 | Fallopian Epithelium | 0.08 | 0.83 | intron | EXOC2 | U |
| 2039 | chr6 | 25262929 | 25263312 | Fallopian Epithelium | 0.17 | 0.92 | Intergenic | LRRC16A | U |
| 2040 | chr11 | 71888432 | 71888781 | Fallopian Epithelium | 0.18 | 0.86 | Intergenic | FOLR1 | U |
| 2041 | chr20 | 23666367 | 23666597 | Fallopian Epithelium | 0.08 | 0.74 | exon, intron | CST4, CST4 | U |
| 2042 | chr7 | 121090 | 121201 | Fallopian Epithelium | 0.32 | 0.84 | Intergenic | FAM20C | U |
| 2043 | chr10 | 95361578 | 95361781 | Fallopian Epithelium | 0.82 | 0.09 | promoter-TSS | RBP4 | M |
| 2044 | chr2 | 120188912 | 120189024 | Fallopian Epithelium | 0.84 | 0.13 | promoter-TSS | TMEM37 | M |
| 2045 | chr11 | 115227865 | 115228020 | Fallopian Epithelium | 0.8 | 0.18 | intron | CADM1 | M |
| 2046 | chr11 | 41480355 | 41480636 | Fallopian Epithelium | 0.78 | 0.17 | intron | LRRC4C | M |
| 2047 | chr5 | 67535502 | 67535751 | Fallopian Epithelium | 0.76 | 0.16 | intron | PIK3R1 | M |
| 2048 | chr5 | 59190362 | 59190426 | Fallopian Epithelium | 0.83 | 0.24 | promoter-TSS | PDE4D | M |
| 2049 | chr5 | 64398711 | 64398764 | Fallopian Epithelium | 0.66 | 0.1 | Intergenic | CWC27 | M |
| 2050 | chr18 | 30137691 | 3013862 | Fallopian Epithelium | 0.73 | 0.2 | Intergenic | LPIN2 | M |
| 2051 | chr6 | 960243131 | 96024529 | Fallopian Epithelium | 0.69 | 0.16 | promoter-TSS | MANEA | M |
| 2052 | chr16 | 3015411 | 3015500 | Fallopian Epithelium | 0.65 | 0.16 | intron, exon | KREMEN2, KREMEN2 | M |
| 2053 | chr5 | 87066677 | 87066817 | Fallopian Epithelium | 0.69 | 0.21 | Intergenic | CCNH | M |
| 2054 | chr12 | 125399869 | 125400014 | Fallopian Epithelium | 0.59 | 0.12 | promoter-TSS | UBC | M |
| 2055 | chr11 | 46696375 | 46696645 | Fallopian Epithelium | 0.66 | 0.19 | exon | ATG13 | M |
| 2056 | chr5 | 47276031 | 47276329 | Fallopian Epithelium | 0.64 | 0.18 | intron | TNFRSF21 | M |
| 2057 | chr1 | 45279151 | 45279278 | Fallopian Epithelium | 0.58 | 0.16 | intron | BTBD19 | M |
| 2058 | chrX | 86773263 | 86773514 | Fallopian Epithelium | 0.71 | 0.29 | intron | KLHL4 | M |
| 2059 | chr4 | 83482420 | 83482509 | Fallopian Epithelium | 0.62 | 0.23 | intron | TMEM150C | M |
| 2060 | chr21 | 46898103 | 46898359 | Fallopian Epithelium | 0.55 | 0.16 | intron | COL18A1 | M |
| 2061 | chrX | 24483799 | 24484000 | Fallopian Epithelium | 0.54 | 0.22 | intron | PDK3 | M |
| 2062 | chr17 | 16284988 | 16285336 | Fallopian Epithelium | 0.69 | 0.05 | exon, intron | UBB, UBB | M |
| 2063 | chr16 | 10274415 | 10274633 | Fallopian Epithelium | 0.76 | 0.14 | intron | GRIN2A | M |
| 2064 | chr5 | 59187838 | 59188013 | Fallopian Epithelium | 0.78 | 0.17 | intron | PDE4D | M |
| 2065 | chr17 | 48619146 | 48619218 | Fallopian Epithelium | 0.61 | 0.08 | intron | EPN3 | M |
| 2066 | chr3 | 151985697 | 151985919 | Fallopian Epithelium | 0.62 | 0.16 | promoter-TSS | MBNL1 | M |
| 2067 | chr14 | 105189951 | 105190016 | Fallopian Epithelium | 0.59 | 0.17 | promoter-TSS | ADSSL1 | M |
| 2068 | chr19 | 391803443 | 39180416 | Kidney Epithelium | 0.09 | 0.96 | intron | ACTN4 | U |
| 2069 | chr3 | 125062531 | 125062912 | Kidney Epithelium | 0.07 | 0.94 | intron | ZNF148 | U |
| 2070 | chr7 | 12583963 | 12584062 | Kidney Epithelium | 0.08 | 0.88 | Intergenic | SCIN | U |
| 2071 | chr2 | 39015988 | 39016238 | Kidney Epithelium | 0.15 | 0.95 | Intergenic | GEMIN6 | U |
| 2072 | chr7 | 87640622 | 87640841 | Kidney Epithelium | 0.15 | 0.93 | intron | ADAM22 | U |
| 2073 | chr19 | 80649073 | 8065002 | Kidney Epithelium | 0.14 | 0.9 | intron | ELAVL1 | U |
| 2074 | chr2 | 203529169 | 203529527 | Kidney Epithelium | 0.16 | 0.92 | intron | FAM1178 | U |
| 2075 | chr1 | 70661377 | 70661570 | Kidney Epithelium | 0.21 | 0.96 | intron | LRRC40 | U |
| 2076 | chr4 | 286423783 | 28642647 | Kidney Epithelium | 0.15 | 0.9 | Intergenic | MIR4275 | U |
| 2077 | chr6 | 241052 | 241153 | Kidney Epithelium | 0.21 | 0.92 | Intergenic | DUSP22 | U |
| 2078 | chr5 | 112953981 | 112954100 | Kidney Epithelium | 0.22 | 0.93 | Intergenic | YTHDC2 | U |
| 2079 | chr10 | 972671 | 972944 | Kidney Epithelium | 0.23 | 0.92 | intron | LARP4B | U |
| 2080 | chr10 | 99251294 | 99251663 | Kidney Epithelium | 0.3 | 0.93 | intron | MMS19 | U |
| 2081 | chr21 | 35212230 | 35212414 | Kidney Epithelium | 0.06 | 0.91 | TTS, intron | ITSN1, ITSN1 | U |
| 2082 | chr2 | 99302337 | 99302637 | Kidney Epithelium | 0.09 | 0.91 | intron | MGAT4A | U |
| 2083 | chr5 | 40996004 | 40996475 | Kidney Epithelium | 0.1 | 0.89 | Intergenic | MROH2B | U |
| 2084 | chr15 | 61181729 | 61181822 | Kidney Epithelium | 0.08 | 0.86 | intron | RORA | U |
| 2085 | chr1 | 96985133 | 96985236 | Kidney Epithelium | 0.17 | 0.93 | Intergenic | PTBP2 | U |
| 2086 | chr6 | 170664874 | 170565152 | Kidney Epithelium | 0.13 | 0.89 | intron | LOC154449 | U |
| 2087 | chr8 | 116559924 | 116560157 | Kidney Epithelium | 0.16 | 0.9 | intron | TRPS1 | U |
| 2088 | chr20 | 3371067 | 3371304 | Kidney Epithelium | 0.17 | 0.91 | intron | C20orf194 | U |
| 2089 | chr7 | 68500931 | 68500967 | Kidney Epithelium | 0.12 | 0.85 | Intergenic | AUTS2 | U |
| 2090 | chr6 | 153471717 | 153471870 | Kidney Epithelium | 0.1 | 0.83 | Intergenic | RGS17 | U |
| 2091 | chr11 | 11626479 | 11626712 | Kidney Epithelium | 0.15 | 0.88 | intron | GALNT18 | U |
| 2092 | chr4 | 379846141 | 37984861 | Kidney Epithelium | 0.16 | 0.89 | intron | TBC1D1 | U |
| 2093 | chr3 | 153086894 | 153087092 | Kidney Epithelium | 0.16 | 0.88 | Intergenic | C3orf79 | U |
| 2094 | chr13 | 23423085 | 23423309 | Kidney Epithelium | 0.07 | 0.79 | Intergenic | BASP1P1 | U |
| 2095 | chr3 | 432610183 | 43261380 | Kidney Epithelium | 0.18 | 0.9 | Intergenic | SNRK | U |
| 2096 | chr12 | 124388479 | 124388930 | Kidney Epithelium | 0.12 | 0.84 | intron | DNAH10 | U |
| 2097 | chr2 | 236883965 | 236884437 | Kidney Epithelium | 0.19 | 0.91 | intron | AGAP1 | U |
| 2098 | chr7 | 151151464 | 151151679 | Kidney Epithelium | 0.19 | 0.9 | Intergenic | CRYGN | U |
| 2099 | chr3 | 56769039 | 56769267 | Kidney Epithelium | 0.14 | 0.85 | intron | ARHGEF3 | U |
| 2100 | chr3 | 1927783553 | 192778486 | Kidney Epithelium | 0.2 | 0.9 | Intergenic | MB21D2 | U |
| 2101 | chr2 | 240722873 | 240723010 | Kidney Epithelium | 0.17 | 0.87 | TTS | LOC150935 | U |
| 2102 | chr1 | 5902819 | 5902963 | Kidney Epithelium | 0.13 | 0.82 | Intergenic | MIR4689 | U |
| 2103 | chr2 | 1278957001 | 127895954 | Kidney Epithelium | 0.22 | 0.91 | Intergenic | BIN1 | U |
| 2104 | chr5 | 180015927 | 180016082 | Kidney Epithelium | 0.2 | 0.88 | Intergenic | SCGB3A1 | U |
| 2105 | chr6 | 153471499 | 153471660 | Kidney Epithelium | 0.13 | 0.81 | Intergenic | RGS17 | U |
| 2106 | chr19 | 38443593 | 38443789 | Kidney Epithelium | 0.23 | 0.91 | intron | SIPA1L3 | U |
| 2107 | chr19 | 18595135 | 18595420 | Kidney Epithelium | 0.22 | 0.9 | intron | ELL | U |
| 2108 | chr13 | 76871766 | 76871871 | Kidney Epithelium | 0.24 | 0.91 | Intergenic | C13orf45 | U |
| 2109 | chr15 | 99943993 | 99944174 | Kidney Epithelium | 0.23 | 0.87 | Intergenic | HSP90B2P | U |
| 2110 | chr13 | 23422878 | 23422953 | Kidney Epithelium | 0.09 | 0.75 | Intergenic | BASP1P1 | U |
| 2111 | chr17 | 1210620 | 1210755 | Kidney Epithelium | 0.21 | 0.87 | Intergenic | TUSC5 | U |
| 2112 | chr17 | 1299134 | 1299234 | Kidney Epithelium | 0.26 | 0.91 | intron | YWHAE | U |
| 2113 | chr10 | 1514522 | 1514697 | Kidney Epithelium | 0.22 | 0.87 | intron | ADARB2 | U |
| 2114 | chr7 | 1506840713 | 150684260 | Kidney Epithelium | 0.23 | 0.88 | Intergenic | NOS3 | U |
| 2115 | chr10 | 132891205 | 132891551 | Kidney Epithelium | 0.25 | 0.9 | exon | TCERG1L | U |
| 2116 | chr11 | 64733146 | 64733345 | Kidney Epithelium | 0.2 | 0.84 | Intergenic | C11orf85 | U |
| 2117 | chr15 | 69370689 | 69371017 | Kidney Epithelium | 0.27 | 0.91 | intron | MIR548H4 | U |
| 2118 | chr12 | 127756296 | 127756380 | Kidney Epithelium | 0.27 | 0.9 | Intergenic | LOC440117 | U |
| 2119 | chr12 | 131507099 | 131507260 | Kidney Epithelium | 0.24 | 0.87 | intron | GPR133 | U |
| 2120 | chr19 | 39529370 | 39529595 | Kidney Epithelium | 0.27 | 0.9 | Intergenic | FBXO27 | U |
| 2121 | chr4 | 129506564 | 129506833 | Kidney Epithelium | 0.3 | 0.93 | Intergenic | PHF17 | U |
| 2122 | chr13 | 33764942 | 33765270 | Kidney Epithelium | 0.27 | 0.9 | intron | STARD13 | U |
| 2123 | chr3 | 167925108 | 167925496 | Kidney Epithelium | 0.27 | 0.9 | Intergenic | EGFEM1P | U |
| 2124 | chr17 | 1200827 | 1201001 | Kidney Epithelium | 0.24 | 0.86 | intron | TUSC5 | U |
| 2125 | chr5 | 79794683 | 79794920 | Kidney Epithelium | 0.23 | 0.85 | intron | FAM151B | U |
| 2126 | chr5 | 2237991 | 2238213 | Kidney Epithelium | 0.24 | 0.85 | Intergenic | IRX4 | U |
| 2127 | chr16 | 86523650 | 86523906 | Kidney Epithelium | 0.18 | 0.79 | intron | FENDRR | U |
| 2128 | chr2 | 227191218 | 227191551 | Kidney Epithelium | 0.31 | 0.92 | Intergenic | LOC646736 | U |
| 2129 | chr3 | 11111968 | 11112104 | Kidney Epithelium | 0.28 | 0.88 | Intergenic | SLC6A1-AS1 | U |
| 2130 | chr10 | 80538472 | 80538790 | Kidney Epithelium | 0.26 | 0.86 | Intergenic | ZMIZ1-AS1 | U |
| 2131 | chr11 | 27970403 | 27970759 | Kidney Epithelium | 0.27 | 0.87 | Intergenic | MIR610 | U |
| 2132 | chr17 | 32956139 | 32956404 | Kidney Epithelium | 0.25 | 0.84 | exon, intron | TMEM132E, TMEM13 | U |
| 2133 | chr10 | 13763955 | 13764295 | Kidney Epithelium | 0.26 | 0.85 | intron | FRMD4A | U |
| 2134 | chr3 | 155268258 | 155268686 | Kidney Epithelium | 0.34 | 0.93 | intron | PLCH1 | U |
| 2135 | chr14 | 95215739 | 95215812 | Kidney Epithelium | 0.28 | 0.86 | Intergenic | GSC | U |
| 2136 | chr15 | 49183985 | 49184348 | Kidney Epithelium | 0.35 | 0.93 | intron | SHC4 | U |
| 2137 | chr18 | 11993675 | 11993982 | Kidney Epithelium | 0.33 | 0.86 | intron | IMPA2 | U |
| 2138 | chr16 | 71872180 | 71872562 | Kidney Epithelium | 0.32 | 0.88 | Intergenic | ATXN1L | U |
| 2139 | chr2 | 1316758813 | 131676090 | Kidney Epithelium | 0.3 | 0.85 | intron | ARHGEF4 | U |
| 2140 | chr9 | 1347199223 | 134720096 | Kidney Epithelium | 0.34 | 0.87 | Intergenic | RAPGEF1 | U |
| 2141 | chr4 | 1551616 | 1551924 | Kidney Epithelium | 0.37 | 0.9 | Intergenic | FAM53A | U |
| 2142 | chr16 | 22937329 | 22937400 | Kidney Epithelium | 0.14 | 0.85 | Intergenic | HS3ST2 | U |
| 2143 | chr8 | 68323403 | 68323481 | Kidney Epithelium | 0.18 | 0.89 | Intergenic | ARFGEF1 | U |
| 2144 | chr21 | 47049353 | 47049460 | Kidney Epithelium | 0.32 | 0.88 | Intergenic | SLC19A1 | U |
| 2145 | chr18 | 42290985 | 42291089 | Kidney Epithelium | 0.06 | 0.94 | intron | SETBP1 | U |
| 2146 | chr16 | 12609458 | 12609723 | Kidney Epithelium | 0.08 | 0.91 | intron | SNX29 | U |
| 2147 | chr6 | 96084977 | 96085303 | Kidney Epithelium | 0.13 | 0.93 | Intergenic | MANEA | U |
| 2148 | chr18 | 76151081 | 76151263 | Kidney Epithelium | 0.2 | 0.94 | Intergenic | SALL3 | U |
| 2149 | chr20 | 26118583 | 26118677 | Kidney Epithelium | 0.12 | 0.85 | Intergenic | NCOR1P1 | U |
| 2150 | chr16 | 74659275 | 74659531 | Kidney Epithelium | 0.23 | 0.93 | intron | RFWD3 | U |
| 2151 | chr6 | 149868192 | 149868428 | Kidney Epithelium | 0.24 | 0.94 | Intergenic | PPIL4 | U |
| 2152 | chr10 | 119543627 | 119543965 | Kidney Epithelium | 0.24 | 0.91 | Intergenic | EMX2OS | U |
| 2153 | chr17 | 80100626 | 80100869 | Kidney Epithelium | 0.27 | 0.93 | intron | CCDC57 | U |
| 2154 | chr11 | 35515942 | 35516176 | Kidney Epithelium | 0.29 | 0.95 | intron | PAMR1 | U |
| 2155 | chr5 | 55330568 | 55330869 | Kidney Epithelium | 0.29 | 0.94 | Intergenic | IL6ST | U |
| 2156 | chr6 | 15525028 | 15525517 | Kidney Epithelium | 0.3 | 0.95 | intron | DTNBP1 | U |
| 2157 | chr1 | 117715692 | 117715875 | Kidney Epithelium | 0.11 | 0.93 | intron | VTCN1 | U |
| 2158 | chr9 | 855579513 | 85558014 | Kidney Epithelium | 0.11 | 0.88 | Intergenic | RASEF | U |
| 2159 | chr13 | 49397756 | 49397983 | Kidney Epithelium | 0.15 | 0.92 | Intergenic | CYSLTR2 | U |
| 2160 | chr14 | 62056738 | 62056968 | Kidney Epithelium | 0.09 | 0.86 | intron | FLI22447 | U |
| 2161 | chr13 | 25026065 | 25026411 | Kidney Epithelium | 0.17 | 0.94 | intron | PARP4 | U |
| 2162 | chr5 | 16681956 | 16682099 | Kidney Epithelium | 0.13 | 0.89 | exon | MYO10 | U |
| 2163 | chr16 | 12084268 | 12084506 | Kidney Epithelium | 0.16 | 0.92 | intron | SNX29 | U |
| 2164 | chr11 | 939169 | 939468 | Kidney Epithelium | 0.17 | 0.93 | intron | AP2A2 | U |
| 2165 | chr7 | 151781437 | 151781869 | Kidney Epithelium | 0.18 | 0.93 | intron | GALNT11 | U |
| 2166 | chr7 | 47605153 | 47605261 | Kidney Epithelium | 0.16 | 0.9 | intron | TNS3 | U |
| 2167 | chr8 | 1132808 | 1132948 | Kidney Epithelium | 0.14 | 0.88 | Intergenic | LOC286083 | U |
| 2168 | chr1 | 6750284 | 6750650 | Kidney Epithelium | 0.17 | 0.91 | intron | DNAJC11 | U |
| 2169 | chr17 | 36967440 | 36967851 | Kidney Epithelium | 0.2 | 0.94 | intron | CWC25 | U |
| 2170 | chr12 | 4554786 | 4554972 | Kidney Epithelium | 0.16 | 0.89 | promoter-TSS | FGF6 | U |
| 2171 | chr7 | 8175827 | 8176181 | Kidney Epithelium | 0.18 | 0.91 | intron | ICA1 | U |
| 2172 | chr16 | 81765201 | 81765593 | Kidney Epithelium | 0.22 | 0.94 | Intergenic | PLCG2 | U |
| 2173 | chr9 | 92053603 | 92053682 | Kidney Epithelium | 0.21 | 0.92 | intron | SEMA4D | U |
| 2174 | chr22 | 33952731 | 33952885 | Kidney Epithelium | 0.18 | 0.88 | intron | LARGE | U |
| 2175 | chr7 | 156883572 | 156883759 | Kidney Epithelium | 0.2 | 0.9 | Intergenic | UBE3C | U |
| 2176 | chr5 | 71714704 | 71714919 | Kidney Epithelium | 0.22 | 0.92 | Intergenic | ZNF366 | U |
| 2177 | chr4 | 187564589 | 187564847 | Kidney Epithelium | 0.21 | 0.91 | intron | FAT1 | U |
| 2178 | chr9 | 97412072 | 97412253 | Kidney Epithelium | 0.23 | 0.92 | Intergenic | FBP1 | U |
| 2179 | chr1 | 46131210 | 46131670 | Kidney Epithelium | 0.22 | 0.91 | intron | GPBP1L1 | U |
| 2180 | chr19 | 31213711 | 31214034 | Kidney Epithelium | 0.17 | 0.85 | Intergenic | ZNF536 | U |
| 2181 | chr7 | 157427089 | 157427365 | Kidney Epithelium | 0.19 | 0.86 | intron | PTPRN2 | U |
| 2182 | chr22 | 45613514 | 45613840 | Kidney Epithelium | 0.24 | 0.91 | intron | KIAA0930 | U |
| 2183 | chr7 | 157280199 | 157280369 | Kidney Epithelium | 0.19 | 0.85 | Intergenic | MIR153-2 | U |
| 2184 | chr12 | 57211212 | 57211488 | Kidney Epithelium | 0.26 | 0.91 | Intergenic | HSD1786 | U |
| 2185 | chr4 | 1534879 | 1535047 | Kidney Epithelium | 0.26 | 0.9 | Intergenic | CRIPAK | U |
| 2186 | chr14 | 101492031 | 101492265 | Kidney Epithelium | 0.27 | 0.91 | promoter-TSS | MIR323A | U |
| 2187 | chr12 | 131572350 | 131572700 | Kidney Epithelium | 0.23 | 0.87 | intron | GPR133 | U |
| 2188 | chr2 | 148776876 | 148776957 | Kidney Epithelium | 0.24 | 0.85 | intron | ORC4 | U |
| 2189 | chr7 | 120477404 | 120477662 | Kidney Epithelium | 0.32 | 0.93 | intron | TSPAN12 | U |
| 2190 | chr19 | 33584939 | 33585374 | Kidney Epithelium | 0.33 | 0.94 | exon, intron | GPATCH1, GPATCH1 | U |
| 2191 | chr14 | 33564388 | 33564849 | Kidney Epithelium | 0.25 | 0.86 | intron | NPAS3 | U |
| 2192 | chr16 | 72038677 | 72039042 | Kidney Epithelium | 0.32 | 0.89 | Intergenic | DHODH | U |
| 2193 | chr8 | 143662736 | 143662925 | Kidney Epithelium | 0.29 | 0.85 | Intergenic | ARC | U |
| 2194 | chr7 | 5886722 | 5886892 | Kidney Epithelium | 0.35 | 0.87 | exon | ZNF815P | U |
| 2195 | chr5 | 72597296 | 72597412 | Kidney Epithelium | 0.83 | 0.15 | Intergenic | TMEM174 | M |
| 2196 | chr5 | 72597061 | 72597232 | Kidney Epithelium | 0.85 | 0.24 | Intergenic | TMEM174 | M |
| 2197 | chr11 | 3465188 | 3465549 | Kidney Epithelium | 0.71 | 0.1 | Intergenic | OR7E12P | M |
| 2198 | chr1 | 47912004 | 47912340 | Kidney Epithelium | 0.81 | 0.21 | Intergenic | FOXD2 | M |
| 2199 | chr7 | 27231866 | 27232178 | Kidney Epithelium | 0.68 | 0.15 | Intergenic | HOXA11-AS | M |
| 2200 | chr7 | 128520172 | 128520450 | Kidney Epithelium | 0.63 | 0.11 | intron | KCP | M |
| 2201 | chr5 | 72596986 | 72597052 | Kidney Epithelium | 0.68 | 0.18 | Intergenic | TMEM174 | M |
| 2202 | chrX | 10050924 | 10051089 | Kidney Epithelium | 0.7 | 0.21 | intron | WWC3 | M |
| 2203 | chr12 | 54371022 | 54371327 | Kidney Epithelium | 0.65 | 0.19 | TTS | HOXC11 | M |
| 2204 | chr13 | 27077217 | 27077322 | Kidney Epithelium | 0.72 | 0.27 | Intergenic | WASF3 | M |
| 2205 | chr16 | 89119471 | 89119573 | Kidney Epithelium | 0.67 | 0.23 | Intergenic | ACSF3 | M |
| 2206 | chr6 | 134215938 | 134216150 | Kidney Epithelium | 0.61 | 0.17 | exon | TCF21 | M |
| 2207 | chr16 | 89070761 | 89070871 | Kidney Epithelium | 0.63 | 0.2 | Intergenic | CBFA2T3 | M |
| 2208 | chr11 | 9340682 | 9340906 | Kidney Epithelium | 0.59 | 0.2 | Intergenic | TMEM418 | M |
| 2209 | chr20 | 61926633 | 61926785 | Kidney Epithelium | 0.58 | 0.25 | intron | COL20A1 | M |
| 2210 | chr5 | 72677395 | 72677750 | Kidney Epithelium | 0.85 | 0.12 | Intergenic | FOXD1 | M |
| 2211 | chr1 | 47911646 | 47911941 | Kidney Epithelium | 0.79 | 0.16 | Intergenic | FOXD2 | M |
| 2212 | chr12 | 54500128 | 54500244 | Kidney Epithelium | 0.74 | 0.12 | intron | FLJ12825 | M |
| 2213 | chr6 | 107956154 | 107956224 | Kidney Epithelium | 0.71 | 0.19 | exon | SOBP | M |
| 2214 | chr7 | 128808991 | 128809458 | Kidney Epithelium | 0.6 | 0.08 | exon | TSPAN33 | M |
| 2215 | chr7 | 95026106 | 95026249 | Kidney Epithelium | 0.73 | 0.22 | promoter-TSS | PON3 | M |
| 2216 | chr6 | 134214794 | 134215201 | Kidney Epithelium | 0.75 | 0.24 | exon | TCF21 | M |
| 2217 | chr1 | 47911295 | 47911512 | Kidney Epithelium | 0.57 | 0.08 | Intergenic | FOXD2 | M |
| 2218 | chr1 | 47911102 | 47911260 | Kidney Epithelium | 0.69 | 0.22 | Intergenic | FOXD2 | M |
| 2219 | chr2 | 237080546 | 237080908 | Kidney Epithelium | 0.59 | 0.15 | Intergenic | GBX2 | M |
| 2220 | chr3 | 189704095 | 189704171 | Bladder Epithelium | 0.05 | 0.92 | intron | LEPREL1 | U |
| 2221 | chr7 | 97408637 | 97408740 | Bladder Epithelium | 0.11 | 0.94 | Intergenic | TAC1 | U |
| 2222 | chr1 | 34004606 | 34004728 | Bladder Epithelium | 0.08 | 0.9 | intron | CSMD2 | U |
| 2223 | chr10 | 111585736 | 111585935 | Bladder Epithelium | 0.13 | 0.92 | Intergenic | XPNPEP1 | U |
| 2224 | chr2 | 152635949 | 152636402 | Bladder Epithelium | 0.14 | 0.93 | Intergenic | NEB | U |
| 2225 | chr2 | 113715851 | 113716004 | Bladder Epithelium | 0.13 | 0.9 | Intergenic | IL36G | U |
| 2226 | chr10 | 1339932211 | 133993303 | Bladder Epithelium | 0.15 | 0.91 | intron | JAKMIP3 | U |
| 2227 | chr16 | 32889552 | 32889613 | Bladder Epithelium | 0.16 | 0.91 | intron | SLC6A10P | U |
| 2228 | chr8 | 62146817 | 62146949 | Bladder Epithelium | 0.08 | 0.83 | Intergenic | CLVS1 | U |
| 2229 | chr2 | 48736358 | 48736768 | Bladder Epithelium | 0.14 | 0.89 | intron | PPP1R21 | U |
| 2230 | chr14 | 81856494 | 81856862 | Bladder Epithelium | 0.2 | 0.93 | intron | STON2 | U |
| 2231 | chr3 | 70922903 | 70923139 | Bladder Epithelium | 0.16 | 0.88 | Intergenic | MIR1284 | U |
| 2232 | chr19 | 56725662 | 56725847 | Bladder Epithelium | 0.17 | 0.87 | Intergenic | ZSCAN5A | U |
| 2233 | chr4 | 2961841 | 2962322 | Bladder Epithelium | 0.31 | 0.92 | intron | NOP14 | U |
| 2234 | chr19 | 49305765 | 49305999 | Bladder Epithelium | 0.11 | 0.91 | intron | BCAT2 | U |
| 2235 | chr1 | 33609720 | 33609941 | Bladder Epithelium | 0.13 | 0.92 | Intergenic | TRIM62 | U |
| 2236 | chr7 | 34214882 | 34215193 | Bladder Epithelium | 0.11 | 0.89 | Intergenic | BMPER | U |
| 2237 | chr2 | 131085456 | 131085544 | Bladder Epithelium | 0.08 | 0.85 | Intergenic | CCDC115 | U |
| 2238 | chr16 | 33785709 | 33785771 | Bladder Epithelium | 0.14 | 0.9 | Intergenic | LINC00273 | U |
| 2239 | chr16 | 16033571 | 1603506 | Bladder Epithelium | 0.14 | 0.9 | intron | TMEM204 | U |
| 2240 | chr19 | 53463258 | 53463449 | Bladder Epithelium | 0.18 | 0.94 | intron | ZNF816 | U |
| 2241 | chr10 | 124054428 | 124054658 | Bladder Epithelium | 0.09 | 0.85 | intron | BTBD16 | U |
| 2242 | chr2 | 189648457 | 189648529 | Bladder Epithelium | 0.18 | 0.93 | intron | DIRC1 | U |
| 2243 | chr2 | 131085546 | 131085628 | Bladder Epithelium | 0.05 | 0.8 | Intergenic | CCDC115 | U |
| 2244 | chr13 | 20969691 | 20969953 | Bladder Epithelium | 0.12 | 0.87 | Intergenic | MIR4499 | U |
| 2245 | chr1 | 2032732301 | 203273497 | Bladder Epithelium | 0.17 | 0.92 | intron | LOC730227 | U |
| 2246 | chr4 | 121662379 | 121662872 | Bladder Epithelium | 0.15 | 0.9 | intron | PRDM5 | U |
| 2247 | chr10 | 3984706 | 3985205 | Bladder Epithelium | 0.12 | 0.87 | Intergenic | KLF6 | U |
| 2248 | chr17 | 77844945 | 77845047 | Bladder Epithelium | 0.18 | 0.92 | Intergenic | CBX4 | U |
| 2249 | chr9 | 135812999 | 135813249 | Bladder Epithelium | 0.17 | 0.91 | intron | TSC1 | U |
| 2250 | chr10 | 28956172 | 28956309 | Bladder Epithelium | 0.15 | 0.88 | Intergenic | BAMBI | U |
| 2251 | chr7 | 116048933 | 116049078 | Bladder Epithelium | 0.19 | 0.92 | Intergenic | CAV2 | U |
| 2252 | chr11 | 75405128 | 75405563 | Bladder Epithelium | 0.14 | 0.87 | Intergenic | MOGAT2 | U |
| 2253 | chr3 | 120097307 | 120097436 | Bladder Epithelium | 0.2 | 0.92 | Intergenic | MIR198 | U |
| 2254 | chr10 | 33311432 | 33311625 | Bladder Epithelium | 0.14 | 0.86 | Intergenic | ITGB1 | U |
| 2255 | chr3 | 128143761 | 128144040 | Bladder Epithelium | 0.17 | 0.89 | Intergenic | DNAJB8-AS1 | U |
| 2256 | chr18 | 7048033 | 7048313 | Bladder Epithelium | 0.1 | 0.82 | intron | LAMA1 | U |
| 2257 | chr20 | 50247316 | 50247452 | Bladder Epithelium | 0.22 | 0.93 | intron | ATP9A | U |
| 2258 | chr2 | 55180750 | 55180954 | Bladder Epithelium | 0.23 | 0.94 | intron | EML6 | U |
| 2259 | chr13 | 43835051 | 43835441 | Bladder Epithelium | 0.13 | 0.84 | intron | ENOX1 | U |
| 2260 | chr11 | 107416771 | 107417198 | Bladder Epithelium | 0.16 | 0.87 | intron | ALKBH8 | U |
| 2261 | chr1 | 15232920 | 15233019 | Bladder Epithelium | 0.21 | 0.91 | intron | KAZN | U |
| 2262 | chr18 | 8552688 | 8552889 | Bladder Epithelium | 0.19 | 0.89 | Intergenic | RAB12 | U |
| 2263 | chr4 | 37528843 | 37529269 | Bladder Epithelium | 0.17 | 0.87 | intron | C4orf19 | U |
| 2264 | chr4 | 1297129621 | 129713394 | Bladder Epithelium | 0.2 | 0.9 | Intergenic | PHF17 | U |
| 2265 | chr18 | 8458878 | 8459062 | Bladder Epithelium | 0.1 | 0.79 | Intergenic | LOC100192426 | U |
| 2266 | chr1 | 154254086 | 154254277 | Bladder Epithelium | 0.22 | 0.91 | Intergenic | HAX1 | U |
| 2267 | chr1 | 6377471 | 6377733 | Bladder Epithelium | 0.16 | 0.85 | intron | ACOT7 | U |
| 2268 | chr7 | 27063444 | 27063749 | Bladder Epithelium | 0.2 | 0.89 | Intergenic | HOXA1 | U |
| 2269 | chr19 | 46005910 | 46006102 | Bladder Epithelium | 0.16 | 0.84 | TTS | PPM1N | U |
| 2270 | chr10 | 98602423 | 98602685 | Bladder Epithelium | 0.2 | 0.88 | intron | LCOR | U |
| 2271 | chr17 | 7828771 | 7829035 | Bladder Epithelium | 0.23 | 0.91 | intron | KCNAB3 | U |
| 2272 | chr7 | 102380675 | 102381115 | Bladder Epithelium | 0.23 | 0.91 | Intergenic | FAM185A | U |
| 2273 | chr10 | 30453416 | 30453576 | Bladder Epithelium | 0.18 | 0.84 | Intergenic | KIAA1462 | U |
| 2274 | chr2 | 11108531 | 11108714 | Bladder Epithelium | 0.18 | 0.84 | Intergenic | KCNF1 | U |
| 2275 | chr1 | 208227830 | 208228179 | Bladder Epithelium | 0.21 | 0.87 | intron | PLXNA2 | U |
| 2276 | chr2 | 1692526 | 1692911 | Bladder Epithelium | 0.25 | 0.9 | intron | PXDN | U |
| 2277 | chr15 | 67441115 | 67441518 | Bladder Epithelium | 0.25 | 0.9 | intron | SMAD3 | U |
| 2278 | chr11 | 131515832 | 131516239 | Bladder Epithelium | 0.2 | 0.85 | intron | NTM | U |
| 2279 | chr18 | 33741240 | 33741681 | Bladder Epithelium | 0.27 | 0.92 | intron | ELP2 | U |
| 2280 | chr10 | 124049993 | 124050067 | Bladder Epithelium | 0.15 | 0.79 | intron | BTBD16 | U |
| 2281 | chr15 | 79471899 | 79472016 | Bladder Epithelium | 0.21 | 0.85 | Intergenic | MIR184 | U |
| 2282 | chr5 | 95992213 | 95992496 | Bladder Epithelium | 0.24 | 0.88 | Intergenic | CAST | U |
| 2283 | chr6 | 1252667 | 1252846 | Bladder Epithelium | 0.27 | 0.9 | Intergenic | FOXQ1 | U |
| 2284 | chr8 | 133936781 | 133937077 | Bladder Epithelium | 0.23 | 0.86 | intron | TG | U |
| 2285 | chr8 | 134917504 | 134917801 | Bladder Epithelium | 0.22 | 0.85 | Intergenic | ST3GAL1 | U |
| 2286 | chr3 | 221574003 | 22157746 | Bladder Epithelium | 0.2 | 0.83 | Intergenic | ZNF385D | U |
| 2287 | chr2 | 101802746 | 101803172 | Bladder Epithelium | 0.25 | 0.88 | Intergenic | TBC1D8 | U |
| 2288 | chr19 | 46006156 | 46006295 | Bladder Epithelium | 0.16 | 0.78 | TTS | PPM1N | U |
| 2289 | chr2 | 44975430 | 44975638 | Bladder Epithelium | 0.32 | 0.93 | intron | CAMKMT | U |
| 2290 | chr18 | 71902900 | 71903228 | Bladder Epithelium | 0.25 | 0.86 | Intergenic | CYB5A | U |
| 2291 | chr8 | 126291702 | 126292152 | Bladder Epithelium | 0.25 | 0.86 | intron | NSMCE2 | U |
| 2292 | chr7 | 53479336 | 53479436 | Bladder Epithelium | 0.29 | 0.88 | Intergenic | POM121L12 | U |
| 2293 | chr8 | 128676466 | 128676733 | Bladder Epithelium | 0.31 | 0.9 | Intergenic | MYC | U |
| 2294 | chr15 | 58727874 | 58728190 | Bladder Epithelium | 0.34 | 0.93 | intron | LIPC | U |
| 2295 | chr1 | 118823577 | 118823826 | Bladder Epithelium | 0.32 | 0.9 | Intergenic | SPAG17 | U |
| 2296 | chr7 | 86668921 | 86669145 | Bladder Epithelium | 0.15 | 0.72 | intron | KIAA1324L | U |
| 2297 | chr14 | 103717739 | 103718125 | Bladder Epithelium | 0.36 | 0.93 | Intergenic | LINC00605 | U |
| 2298 | chr1 | 202026867 | 202027318 | Bladder Epithelium | 0.36 | 0.86 | Intergenic | ELF3 | U |
| 2299 | chr10 | 9271378 | 9271573 | Bladder Epithelium | 0.09 | 0.89 | Intergenic | GATA3 | U |
| 2300 | chr13 | 111691869 | 111692013 | Bladder Epithelium | 0.21 | 0.92 | Intergenic | ARHGEF7 | U |
| 2301 | chr1 | 240922366 | 240922535 | Bladder Epithelium | 0.15 | 0.87 | Intergenic | GREM2 | U |
| 2302 | chr7 | 140510166 | 140510603 | Bladder Epithelium | 0.19 | 0.91 | intron | BRAF | U |
| 2303 | chr2 | 124447715 | 124447893 | Bladder Epithelium | 0.11 | 0.8 | Intergenic | CNTNAP5 | U |
| 2304 | chr1 | 184970698 | 184970983 | Bladder Epithelium | 0.1 | 0.93 | Intergenic | FAM129A | U |
| 2305 | chr2 | 135276067 | 135276149 | Bladder Epithelium | 0.13 | 0.92 | intron | TMEM163 | U |
| 2306 | chr15 | 77448649 | 77448987 | Bladder Epithelium | 0.12 | 0.92 | intron | PEAK1 | U |
| 2307 | chr4 | 2961192 | 2961471 | Bladder Epithelium | 0.16 | 0.94 | intron | NOP14 | U |
| 2308 | chr12 | 10267729 | 10267793 | Bladder Epithelium | 0.1 | 0.87 | Intergenic | CLEC7A | U |
| 2309 | chr6 | 34778179 | 34778643 | Bladder Epithelium | 0.16 | 0.93 | intron | UHRF1BP1 | U |
| 2310 | chr15 | 101938761 | 101938872 | Bladder Epithelium | 0.17 | 0.94 | exon, intron | PCSK6 | U |
| 2311 | chr4 | 385688513 | 38569128 | Bladder Epithelium | 0.17 | 0.92 | Intergenic | KLF3 | U |
| 2312 | chr11 | 57500830 | 57501020 | Bladder Epithelium | 0.26 | 0.94 | intron | TMX2-CTNND1 | U |
| 2313 | chr7 | 155581063 | 155581411 | Bladder Epithelium | 0.32 | 0.91 | Intergenic | SHH | U |
| 2314 | chr15 | 81262936 | 81263036 | Bladder Epithelium | 0.09 | 0.91 | Intergenic | MESDC2 | U |
| 2315 | chr17 | 43208654 | 43208757 | Bladder Epithelium | 0.07 | 0.89 | intron | PLCD3 | U |
| 2316 | chr6 | 56894215 | 56894332 | Bladder Epithelium | 0.07 | 0.87 | Intergenic | KIAA1586 | U |
| 2317 | chr18 | 7173233 | 7173411 | Bladder Epithelium | 0.1 | 0.89 | Intergenic | LAMA1 | U |
| 2318 | chr10 | 70184305 | 70184465 | Bladder Epithelium | 0.15 | 0.93 | intron | DNA2 | U |
| 2319 | chr18 | 74246866 | 74247019 | Bladder Epithelium | 0.11 | 0.88 | intron | LOC284276 | U |
| 2320 | chr14 | 102493581 | 102493778 | Bladder Epithelium | 0.09 | 0.86 | intron | DYNC1H1 | U |
| 2321 | chr3 | 184566501 | 184566691 | Bladder Epithelium | 0.16 | 0.92 | intron | VP58 | U |
| 2322 | chr9 | 98119673 | 98119940 | Bladder Epithelium | 0.13 | 0.89 | Intergenic | FANCC | U |
| 2323 | chr14 | 50835876 | 50836194 | Bladder Epithelium | 0.13 | 0.89 | intron | CDKL1 | U |
| 2324 | chr9 | 85843600 | 85843716 | Bladder Epithelium | 0.13 | 0.88 | Intergenic | RASEF | U |
| 2325 | chr16 | 1576680 | 1576810 | Bladder Epithelium | 0.18 | 0.93 | intron, exon | IFT140, IFT140 | U |
| 2326 | chr9 | 98919710 | 98919766 | Bladder Epithelium | 0.16 | 0.9 | Intergenic | LOC158434 | U |
| 2327 | chr14 | 552063703 | 55206541 | Bladder Epithelium | 0.11 | 0.85 | intron | SAMD4A | U |
| 2328 | chr2 | 231604240 | 231604554 | Bladder Epithelium | 0.17 | 0.91 | intron | CAB39 | U |
| 2329 | chr9 | 92717057 | 92717425 | Bladder Epithelium | 0.16 | 0.9 | Intergenic | MIR4290 | U |
| 2330 | chr9 | 15513985 | 15514009 | Bladder Epithelium | 0.16 | 0.89 | Intergenic | PSIP1 | U |
| 2331 | chr14 | 102493027 | 102493519 | Bladder Epithelium | 0.17 | 0.89 | intron | DYNC1H1 | U |
| 2332 | chr2 | 10162285 | 10162472 | Bladder Epithelium | 0.21 | 0.92 | Intergenic | KLF11 | U |
| 2333 | chr5 | 148772552 | 148772628 | Bladder Epithelium | 0.21 | 0.91 | Intergenic | IL17B | U |
| 2334 | chr8 | 53755910 | 53756057 | Bladder Epithelium | 0.22 | 0.9 | Intergenic | NPBWR1 | U |
| 2335 | chr14 | 104047749 | 104048041 | Bladder Epithelium | 0.23 | 0.91 | intron | APOPT1 | U |
| 2336 | chr9 | 36274911 | 36275149 | Bladder Epithelium | 0.27 | 0.94 | intron | GNE | U |
| 2337 | chr3 | 11581015 | 11581215 | Bladder Epithelium | 0.27 | 0.92 | intron | ATG7 | U |
| 2338 | chr12 | 109936018 | 109936169 | Bladder Epithelium | 0.29 | 0.93 | exon | UBE3B | U |
| 2339 | chr16 | 12236884 | 12237154 | Bladder Epithelium | 0.31 | 0.94 | intron | SNX29 | U |
| 2340 | chr16 | 89674297 | 89674436 | Bladder Epithelium | 0.17 | 0.79 | Intergenic | DPEP1 | U |
| 2341 | chr6 | 106582669 | 106582732 | Bladder Epithelium | 0.32 | 0.91 | Intergenic | PRDM1 | U |
| 2342 | chr20 | 43358892 | 43359077 | Bladder Epithelium | 0.3 | 0.89 | Intergenic | WISP2 | U |
| 2343 | chr9 | 90892837 | 90893046 | Bladder Epithelium | 0.28 | 0.87 | Intergenic | SPIN1 | U |
| 2344 | chr20 | 19204280 | 19204438 | Bladder Epithelium | 0.36 | 0.91 | intron | SLC24A3 | U |
| 2345 | chr13 | 114762431 | 114762866 | Bladder Epithelium | 0.34 | 0.88 | intron | RASA3 | U |
| 2346 | chr12 | 54773440 | 54773703 | Bladder Epithelium | 0.79 | 0.03 | intron | ZNF385A | M |
| 2347 | chr7 | 3018797 | 3018918 | Bladder Epithelium | 0.81 | 0.14 | intron | CARD11 | M |
| 2348 | chr3 | 181437662 | 181437830 | Bladder Epithelium | 0.85 | 0.18 | intron | SOX2-OT | M |
| 2349 | chr1 | 2402103 | 2402337 | Bladder Epithelium | 0.82 | 0.21 | Intergenic | PLCH2 | M |
| 2350 | chr17 | 79075542 | 79075780 | Bladder Epithelium | 0.67 | 0.08 | intron | BAIAP2 | M |
| 2351 | chr10 | 105420501 | 105420692 | Bladder Epithelium | 0.73 | 0.09 | intron | SH3PXD2A | M |
| 2352 | chr20 | 34188724 | 34189055 | Bladder Epithelium | 0.87 | 0.09 | Intergenic | SPAG4 | M |
| 2353 | chr10 | 77155143 | 77155405 | Bladder Epithelium | 0.84 | 0.07 | Intergenic | ZNF503 | M |
| 2354 | chr1 | 29586283 | 29586758 | Bladder Epithelium | 0.8 | 0.03 | exon | PTPRU | M |
| 2355 | chr10 | 77155437 | 77155546 | Bladder Epithelium | 0.88 | 0.14 | Intergenic | ZNF503 | M |
| 2356 | chr3 | 181437928 | 181438329 | Bladder Epithelium | 0.85 | 0.11 | intron | SOX2-OT | M |
| 2357 | chr3 | 181445573 | 181445764 | Bladder Epithelium | 0.84 | 0.11 | intron | SOX2-OT | M |
| 2358 | chr17 | 46711051 | 46711447 | Bladder Epithelium | 0.78 | 0.06 | Intergenic | MIR196A1 | M |
| 2359 | chr19 | 36164093 | 36164514 | Bladder Epithelium | 0.73 | 0.05 | promoter-TSS | UPK1A-AS1 | M |
| 2360 | chr17 | 46710934 | 46711040 | Bladder Epithelium | 0.79 | 0.12 | promoter-TSS, Interg [symbol] | MIR196A1, MIR196A1 | M |
| 2361 | chr5 | 42949328 | 42949428 | Bladder Epithelium | 0.81 | 0.16 | Intergenic | LOC648987 | M |
| 2362 | chr5 | 429494523 | 42949800 | Bladder Epithelium | 0.7 | 0.09 | Intergenic | LOC648987 | M |
| 2363 | chr7 | 20832969 | 20833089 | Bladder Epithelium | 0.78 | 0.19 | Intergenic | SP8 | M |
| 2364 | chr7 | 20831202 | 20831374 | Bladder Epithelium | 0.75 | 0.16 | Intergenic | SP8 | M |
| 2365 | chr3 | 181445800 | 181445921 | Bladder Epithelium | 0.66 | 0.08 | intron | SOX2-OT | M |
| 2366 | chr17 | 46713821 | 46714156 | Bladder Epithelium | 0.59 | 0.03 | Intergenic | MIR196A1 | M |
| 2367 | chr17 | 46714169 | 46714313 | Bladder Epithelium | 0.61 | 0.07 | Intergenic | MIR196A1 | M |
| 2368 | chr16 | 89006582 | 89006727 | Bladder Epithelium | 0.56 | 0.07 | intron | CBFA2T3 | M |
| 2369 | chr5 | 50265721 | 50265867 | Bladder Epithelium | 0.58 | 0.12 | Intergenic | PARP8 | M |
| 2370 | chr19 | 863037 | 863158 | Bladder Epithelium | 0.59 | 0.17 | intron, exon | CFD, CFD | M |
| 2371 | chr5 | 58395983 | 58396401 | Prostate Epithelium | 0.08 | 0.93 | intron | PDE4D | U |
| 2372 | chr11 | 82843660 | 82843842 | Prostate Epithelium | 0.03 | 0.88 | Intergenic | PCF11 | U |
| 2373 | chr17 | 9348790 | 9348935 | Prostate Epithelium | 0.13 | 0.9 | intron | STX8 | U |
| 2374 | chr3 | 149001639 | 149001777 | Prostate Epithelium | 0.14 | 0.9 | Intergenic | TM4SF18 | U |
| 2375 | chr12 | 40171398 | 40171588 | Prostate Epithelium | 0.15 | 0.91 | intron | SLC2A13 | U |
| 2376 | chrX | 35518524 | 35518564 | Prostate Epithelium | 0.12 | 0.87 | Intergenic | MAGEB16 | U |
| 2377 | chr8 | 852993 | 853135 | Prostate Epithelium | 0.14 | 0.88 | intron | ERICH1-AS1 | U |
| 2378 | chr3 | 1936756343 | 193675816 | Prostate Epithelium | 0.14 | 0.88 | exon | LOC647323 | U |
| 2379 | chr6 | 720637 | 720835 | Prostate Epithelium | 0.16 | 0.9 | Intergenic | EXOC2 | U |
| 2380 | chr4 | 1003428541 | 100343192 | Prostate Epithelium | 0.2 | 0.93 | intron | ADH7 | U |
| 2381 | chr5 | 1712231471 | 171223507 | Prostate Epithelium | 0.17 | 0.89 | Intergenic | FBXW11 | U |
| 2382 | chr11 | 61868530 | 61868709 | Prostate Epithelium | 0.17 | 0.87 | Intergenic | INCENP | U |
| 2383 | chr15 | 88585662 | 88585850 | Prostate Epithelium | 0.15 | 0.85 | intron | NTRK3 | U |
| 2384 | chr11 | 82843881 | 82843952 | Prostate Epithelium | 0.25 | 0.94 | Intergenic | PCF11 | U |
| 2385 | chr9 | 16204126 | 16204159 | Prostate Epithelium | 0.25 | 0.92 | intron | C9orf92 | U |
| 2386 | chr19 | 51858650 | 51858909 | Prostate Epithelium | 0.21 | 0.85 | intron, promoter-TS [symbol] | ETFB | U |
| 2387 | chr2 | 112285718 | 112285860 | Prostate Epithelium | 0.26 | 0.88 | lIntergenic | LOCS41471 | U |
| 2388 | chr13 | 43922157 | 43922412 | Prostate Epithelium | 0.29 | 0.88 | intron | ENOX1 | U |
| 2389 | chr18 | 13812352 | 13812503 | Prostate Epithelium | 0.24 | 0.81 | Intergenic | MC5R | U |
| 2390 | chr4 | 567379401 | 56738149 | Prostate Epithelium | 0.05 | 0.87 | exon | EXOC1 | U |
| 2391 | chr8 | 52894910 | 52894972 | Prostate Epithelium | 0.09 | 0.9 | Intergenic | PCMTD1 | U |
| 2392 | chr15 | 30146107 | 30146255 | Prostate Epithelium | 0.07 | 0.88 | Intergenic | TJP1 | U |
| 2393 | chr7 | 157423150 | 157423212 | Prostate Epithelium | 0.08 | 0.86 | intron | PTPRN2 | U |
| 2394 | chr3 | 24086762 | 24087106 | Prostate Epithelium | 0.08 | 0.86 | Intergenic | LINC00691 | U |
| 2395 | chr12 | 31624662 | 31625071 | Prostate Epithelium | 0.07 | 0.85 | intron | DENND5B | U |
| 2396 | chr17 | 75679958 | 75680289 | Prostate Epithelium | 0.11 | 0.88 | Intergenic | LOC100507351 | U |
| 2397 | chr8 | 19622714 | 19622802 | Prostate Epithelium | 0.12 | 0.88 | Intergenic | INTS10 | U |
| 2398 | chr1 | 116153836 | 116154186 | Prostate Epithelium | 0.12 | 0.88 | Intergenic | VANGL1 | U |
| 2399 | chr1 | 61105704 | 61106011 | Prostate Epithelium | 0.15 | 0.9 | Intergenic | NFIA | U |
| 2400 | chr1 | 245662442 | 245662843 | Prostate Epithelium | 0.14 | 0.88 | intron | KIF268 | U |
| 2401 | chr15 | 100632520 | 100632750 | Prostate Epithelium | 0.16 | 0.89 | intron | ADAMTS17 | U |
| 2402 | chr21 | 43966798 | 43967117 | Prostate Epithelium | 0.18 | 0.91 | intron | SLC37A1 | U |
| 2403 | chr5 | 61105044 | 61105179 | Prostate Epithelium | 0.18 | 0.89 | Intergenic | C5orf64 | U |
| 2404 | chr16 | 12436822 | 12437045 | Prostate Epithelium | 0.14 | 0.84 | intron | SNX29 | U |
| 2405 | chr15 | 43079249 | 43079532 | Prostate Epithelium | 0.18 | 0.88 | intron | TTBK2 | U |
| 2406 | chr2 | 179830635 | 179830675 | Prostate Epithelium | 0.16 | 0.85 | intron | CCDC141 | U |
| 2407 | chr3 | 41974443 | 41974521 | Prostate Epithelium | 0.22 | 0.91 | intron | ULK4 | U |
| 2408 | chr7 | 84294370 | 84294617 | Prostate Epithelium | 0.18 | 0.87 | Intergenic | SEMA3D | U |
| 2409 | chr12 | 104894025 | 104894291 | Prostate Epithelium | 0.15 | 0.84 | intron | CHST11 | U |
| 2410 | chr15 | 91888665 | 91888766 | Prostate Epithelium | 0.19 | 0.86 | Intergenic | SV2B | U |
| 2411 | chr10 | 123781645 | 123781742 | Prostate Epithelium | 0.2 | 0.86 | intron | TACC2 | U |
| 2412 | chr1 | 180911672 | 180911819 | Prostate Epithelium | 0.22 | 0.88 | intron | KIAA1614 | U |
| 2413 | chr6 | 53824292 | 53824469 | Prostate Epithelium | 0.23 | 0.89 | Intergenic | MLIP-IT1 | U |
| 2414 | chr4 | 2632299 | 2632484 | Prostate Epithelium | 0.22 | 0.88 | intron | FAM193A | U |
| 2415 | chr8 | 130119953 | 130120159 | Prostate Epithelium | 0.22 | 0.87 | Intergenic | LOC728724 | U |
| 2416 | chrX | 106840564 | 106840785 | Prostate Epithelium | 0.17 | 0.82 | Intergenic | PRPS1 | U |
| 2417 | chr2 | 153562909 | 153563239 | Prostate Epithelium | 0.23 | 0.88 | intron | PRPF40A | U |
| 2418 | chr18 | 50966157 | 50966236 | Prostate Epithelium | 0.24 | 0.88 | intron | DCC | U |
| 2419 | chr11 | 111903013 | 11190581 | Prostate Epithelium | 0.2 | 0.84 | Intergenic | CSNK2A3 | U |
| 2420 | chr2 | 862738351 | 86274274 | Prostate Epithelium | 0.23 | 0.87 | intron | POLR1A | U |
| 2421 | chr6 | 73112707 | 73113170 | Prostate Epithelium | 0.22 | 0.86 | TTS | RIMS1 | U |
| 2422 | chr17 | 1338500 | 1338969 | Prostate Epithelium | 0.21 | 0.85 | intron | CRK | U |
| 2423 | chr15 | 53833154 | 53833205 | Prostate Epithelium | 0.25 | 0.88 | intron | WDR72 | U |
| 2424 | chr17 | 53940464 | 53940594 | Prostate Epithelium | 0.27 | 0.9 | Intergenic | PCTP | U |
| 2425 | chr10 | 12316772 | 12316908 | Prostate Epithelium | 0.25 | 0.88 | Intergenic | CAMK1D | U |
| 2426 | chr12 | 45526093 | 45526272 | Prostate Epithelium | 0.22 | 0.85 | Intergenic | RNY5 | U |
| 2427 | chr2 | 179303422 | 179303614 | Prostate Epithelium | 0.27 | 0.9 | intron | PRKRA | U |
| 2428 | chr22 | 48061604 | 48061858 | Prostate Epithelium | 0.18 | 0.81 | Intergenic | FLI46257 | U |
| 2429 | chr7 | 105845720 | 105846055 | Prostate Epithelium | 0.26 | 0.89 | Intergenic | NAMPT | U |
| 2430 | chr6 | 62750149 | 62750498 | Prostate Epithelium | 0.27 | 0.9 | intron | KHDRBS2 | U |
| 2431 | chr8 | 113513406 | 113513591 | Prostate Epithelium | 0.24 | 0.86 | intron | CSMD3 | U |
| 2432 | chr1 | 6977718 | 6977906 | Prostate Epithelium | 0.27 | 0.89 | intron | CAMTA1 | U |
| 2433 | chr1 | 244882534 | 244882840 | Prostate Epithelium | 0.24 | 0.86 | Intergenic | DESI2 | U |
| 2434 | chr21 | 31710901 | 31711343 | Prostate Epithelium | 0.22 | 0.84 | promoter-TSS, Interg [symbol] | KRTAP27-1, KRTAP27- [symbol] | U |
| 2435 | chr7 | 156194279 | 156194629 | Prostate Epithelium | 0.21 | 0.82 | Intergenic | LOC285889 | U |
| 2436 | chr10 | 45084800 | 45084855 | Prostate Epithelium | 0.31 | 0.91 | Intergenic | CXCL12 | U |
| 2437 | chr7 | 84317157 | 84317332 | Prostate Epithelium | 0.25 | 0.85 | Intergenic | SEMA3D | U |
| 2438 | chr14 | 86296858 | 86297151 | Prostate Epithelium | 0.25 | 0.85 | Intergenic | FLRT2 | U |
| 2439 | chr16 | 46964865 | 46965206 | Prostate Epithelium | 0.24 | 0.84 | exon, TTS | GPT2, GPT2 | U |
| 2440 | chr5 | 125574024 | 125574432 | Prostate Epithelium | 0.26 | 0.86 | Intergenic | GRAMD3 | U |
| 2441 | chr1 | 65091636 | 65092134 | Prostate Epithelium | 0.26 | 0.86 | intron | CACHD1 | U |
| 2442 | chr10 | 118060976 | 118061134 | Prostate Epithelium | 0.25 | 0.84 | Intergenic | CCDC172 | U |
| 2443 | chr5 | 32473153 | 32473353 | Prostate Epithelium | 0.29 | 0.88 | Intergenic | ZFB | U |
| 2444 | chr15 | 98646681 | 98646939 | Prostate Epithelium | 0.24 | 0.83 | Intergenic | ARRDC4 | U |
| 2445 | chr7 | 140001344 | 140001590 | Prostate Epithelium | 0.21 | 0.79 | Intergenic | SLC37A3 | U |
| 2446 | chr16 | 68439870 | 68440167 | Prostate Epithelium | 0.24 | 0.82 | intron | SMPD3 | U |
| 2447 | chr10 | 45012731 | 45012938 | Prostate Epithelium | 0.28 | 0.85 | Intergenic | CXCL12 | U |
| 2448 | chr1 | 15405269 | 15405648 | Prostate Epithelium | 0.29 | 0.86 | intron | KAZN | U |
| 2449 | chr7 | 15751920 | 15752419 | Prostate Epithelium | 0.29 | 0.86 | Intergenic | MEOX2 | U |
| 2450 | chr10 | 96379389 | 96379547 | Prostate Epithelium | 0.32 | 0.88 | Intergenic | CYP2C18 | U |
| 2451 | chr2 | 123109569 | 123109735 | Prostate Epithelium | 0.34 | 0.9 | Intergenic | TSN | U |
| 2452 | chr2 | 164456487 | 164456706 | Prostate Epithelium | 0.29 | 0.85 | Intergenic | FIGN | U |
| 2453 | chr7 | 15687776 | 15688035 | Prostate Epithelium | 0.29 | 0.84 | intron | MEOX2 | U |
| 2454 | chr3 | 1280491191 | 128049434 | Prostate Epithelium | 0.32 | 0.87 | intron | EEFSEC | U |
| 2455 | chr8 | 2823032 | 2823355 | Prostate Epithelium | 0.34 | 0.89 | intron | CSMD1 | U |
| 2456 | chr17 | 46267675 | 46267766 | Prostate Epithelium | 0.32 | 0.86 | intron | SKAP1 | U |
| 2457 | chr2 | 30566703 | 30566897 | Prostate Epithelium | 0.32 | 0.86 | Intergenic | LCLAT | U |
| 2458 | chr5 | 18701992 | 18702338 | Prostate Epithelium | 0.28 | 0.82 | Intergenic | CDH18 | U |
| 2459 | chr3 | 160409872 | 160410317 | Prostate Epithelium | 0.35 | 0.89 | Intergenic | ARL14 | U |
| 2460 | chr15 | 69270069 | 69270531 | Prostate Epithelium | 0.32 | 0.86 | intron | NOX5 | U |
| 2461 | chr8 | 139409602 | 139409782 | Prostate Epithelium | 0.34 | 0.87 | intron | FAM1358 | U |
| 2462 | chr6 | 2594303 | 259658 | Prostate Epithelium | 0.29 | 0.82 | Intergenic | DUSP22 | U |
| 2463 | chr9 | 141028240 | 141028487 | Prostate Epithelium | 0.36 | 0.89 | Intergenic | TUBBP5 | U |
| 2464 | chr2 | 79298718 | 79298990 | Prostate Epithelium | 0.35 | 0.88 | Intergenic | REG1B | U |
| 2465 | chr2 | 121296510 | 121296784 | Prostate Epithelium | 0.3 | 0.83 | Intergenic | LOC84931 | U |
| 2466 | chr14 | 101772273 | 101772700 | Prostate Epithelium | 0.31 | 0.84 | Intergenic | MEG9 | U |
| 2467 | chr5 | 147100992 | 147101087 | Prostate Epithelium | 0.35 | 0.87 | intron | JAKMIPZ | U |
| 2468 | chr8 | 40488928 | 40489286 | Prostate Epithelium | 0.34 | 0.86 | intron | ZMAT4 | U |
| 2469 | chr16 | 2879393 | 2879453 | Prostate Epithelium | 0.25 | 0.76 | Intergenic, promote [symbol] | ZG16B, ZG16B | U |
| 2470 | chr17 | 27313786 | 27314111 | Prostate Epithelium | 0.32 | 0.82 | intron | SEZ6 | U |
| 2471 | chr5 | 107758914 | 107759399 | Prostate Epithelium | 0.33 | 0.83 | Intergenic | FBXL17 | U |
| 2472 | chr2 | 177287863 | 177288100 | Prostate Epithelium | 0.35 | 0.83 | Intergenic | MTX2 | U |
| 2473 | chrX | 32439826 | 32440020 | Prostate Epithelium | 0.33 | 0.8 | intron | DMD | U |
| 2474 | chr4 | 8994124 | 8994245 | Prostate Epithelium | 0.38 | 0.81 | Intergenic | LOC650293 | U |
| 2475 | chr19 | 53802404 | 53802595 | Prostate Epithelium | 0.33 | 0.76 | Intergenic | BIRC8 | U |
| 2476 | chr8 | 78853164 | 78853392 | Prostate Epithelium | 0.35 | 0.77 | Intergenic | PKIA | U |
| 2477 | chr2 | 125165678 | 125165753 | Prostate Epithelium | 0.28 | 0.88 | intron | CNTNAP5 | U |
| 2478 | chr2 | 70456017 | 70456294 | Prostate Epithelium | 0.3 | 0.89 | intron | TIA1 | U |
| 2479 | chr5 | 134909476 | 134909839 | Prostate Epithelium | 0.26 | 0.84 | intron | CXCL14 | U |
| 2480 | chr8 | 49703308 | 49703539 | Prostate Epithelium | 0.31 | 0.87 | Intergenic | EFCAB1 | U |
| 2481 | chr7 | 293859 | 294353 | Prostate Epithelium | 0.06 | 0.88 | intron | FAM20C | U |
| 2482 | chr5 | 174920572 | 174921015 | Prostate Epithelium | 0.09 | 0.89 | intron | SEXN1 | U |
| 2483 | chr22 | 32563087 | 32563521 | Prostate Epithelium | 0.08 | 0.86 | Intergenic | C22orf42 | U |
| 2484 | chr4 | 3403136 | 3403532 | Prostate Epithelium | 0.19 | 0.94 | intron | RGS12 | U |
| 2485 | chr19 | 333553623 | 33355622 | Prostate Epithelium | 0.16 | 0.87 | exon, intron | SLC7A9 | U |
| 2486 | chr16 | 56951467 | 56951745 | Prostate Epithelium | 0.18 | 0.88 | Intergenic | HIERPUD1 | U |
| 2487 | chr16 | 49766911 | 49767105 | Prostate Epithelium | 0.09 | 0.88 | intron | ZNF423 | U |
| 2488 | chr21 | 45401010 | 45401133 | Prostate Epithelium | 0.09 | 0.86 | exon, intron | AGPAT3, AGPAT3 | U |
| 2489 | chr22 | 30443171 | 30443234 | Prostate Epithelium | 0.16 | 0.91 | Intergenic | HORMAD2 | U |
| 2490 | chr20 | 38807276 | 38807658 | Prostate Epithelium | 0.13 | 0.88 | Intergenic | MAFB | U |
| 2491 | chr7 | 293593 | 293813 | Prostate Epithelium | 0.23 | 0.86 | intron | FAM20C | U |
| 2492 | chr3 | 182656877 | 182657032 | Prostate Epithelium | 0.3 | 0.91 | Intergenic | DCUN1D1 | U |
| 2493 | chr22 | 39407845 | 39408217 | Prostate Epithelium | 0.3 | 0.91 | Intergenic | APOBEC3C | U |
| 2494 | chr10 | 16955540 | 16955937 | Prostate Epithelium | 0.3 | 0.87 | intron | CUBN | U |
| 2495 | chr4 | 3417482 | 3417774 | Prostate Epithelium | 0.33 | 0.85 | intron | RGS12 | U |
| 2496 | chr5 | 42757274 | 42757687 | Prostate Epithelium | 0.79 | 0.12 | intron | CCDC152 | M |
| 2497 | chr2 | 176995916 | 175996152 | Prostate Epithelium | 0.74 | 0.19 | intron, exon | HOXD8, HOXD8 | M |
| 2498 | chr12 | 54380589 | 54380879 | Prostate Epithelium | 0.72 | 0.19 | intron | HOXC10 | M |
| 2499 | chr13 | 67568304 | 67568519 | Prostate Epithelium | 0.63 | 0.16 | intron | PCDH9 | M |
| 2500 | chr20 | 50416258 | 50416422 | Prostate Epithelium | 0.68 | 0.25 | intron | SALL4 | M |
| 2501 | chr5 | 134913743 | 134914047 | Prostate Epithelium | 0.57 | 0.1 | intron | CXCL14 | M |
| 2502 | chr2 | 176987679 | 176988129 | Prostate Epithelium | 0.85 | 0.07 | exon | HOXD9 | M |
| 2503 | chr7 | 27264717 | 27264947 | Prostate Epithelium | 0.85 | 0.16 | Intergenic | EVX1 | M |
| 2504 | chr2 | 200327108 | 200327237 | Prostate Epithelium | 0.81 | 0.13 | intron | SATB2 | M |
| 2505 | chr14 | 37130146 | 37130511 | Prostate Epithelium | 0.81 | 0.14 | intron | PAX9 | M |
| 2506 | chr3 | 190529745 | 190529905 | Prostate Epithelium | 0.75 | 0.09 | Intergenic | GMNC | M |
| 2507 | chr9 | 98981790 | 98981856 | Prostate Epithelium | 0.83 | 0.19 | Intergenic | HSD17B3 | M |
| 2508 | chr2 | 176937949 | 176938224 | Prostate Epithelium | 0.66 | 0.05 | Intergenic | EVX2 | M |
| 2509 | chr2 | 200327240 | 200327295 | Prostate Epithelium | 0.81 | 0.21 | intron | SATB2 | M |
| 2510 | chr2 | 176964570 | 176964812 | Prostate Epithelium | 0.7 | 0.1 | exon | HOXD12 | M |
| 2511 | chr2 | 176931775 | 176932019 | Prostate Epithelium | 0.67 | 0.09 | Intergenic | EVX2 | M |
| 2512 | chr2 | 176986712 | 176986957 | Prostate Epithelium | 0.68 | 0.1 | promoter-TSS | HOXD9 | M |
| 2513 | chr14 | 37126308 | 37126583 | Prostate Epithelium | 0.67 | 0.1 | promoter-TSS | PAX9 | M |
| 2514 | chr2 | 176963442 | 176963767 | Prostate Epithelium | 0.67 | 0.1 | Intergenic, promote [symbol] | HOXD12, HOXD12 | M |
| 2515 | chr7 | 27196825 | 27196997 | Prostate Epithelium | 0.7 | 0.15 | promoter-TSS | HOXA7 | M |
| 2516 | chr9 | 98981863 | 98982148 | Prostate Epithelium | 0.64 | 0.11 | Intergenic | HSD17B3 | M |
| 2517 | chr7 | 8477434 | 8477782 | Prostate Epithelium | 0.69 | 0.16 | intron | NXPH1 | M |
| 2518 | chr5 | 1888022 | 1888108 | Prostate Epithelium | 0.68 | 0.17 | Intergenic | IRX4 | M |
| 2519 | chr17 | 80273966 | 80274192 | Prostate Epithelium | 0.68 | 0.2 | intron | CD7 | M |
| 2520 | chr4 | 111540786 | 111541016 | Prostate Epithelium | 0.65 | 0.19 | intron | PITX2 | M |
| 2521 | chr6 | 35058103 | 35058216 | Breast Basal Epithelium | 0.08 | 0.95 | exon | ANKS1A | U |
| 2522 | chr11 | 66639005 | 66639476 | Breast Basal Epithelium | 0.09 | 0.94 | exon, intron | PC | U |
| 2523 | chr16 | 81390362 | 81390581 | Breast Basal Epithelium | 0.1 | 0.94 | intron | GAN | U |
| 2524 | chr12 | 120656820 | 120656995 | Breast Basal Epithelium | 0.12 | 0.94 | intron | PXA | U |
| 2525 | chr19 | 16618102 | 16618214 | Breast Basal Epithelium | 0.12 | 0.93 | intron | C19orf44 | U |
| 2526 | chr15 | 58833989 | 58834171 | Breast Basal Epithelium | 0.14 | 0.94 | exon, intron | LIPC | U |
| 2527 | chr6 | 35057138 | 35057474 | Breast Basal Epithelium | 0.14 | 0.93 | exon | ANKS1A | U |
| 2528 | chr20 | 3171367 | 3171566 | Breast Basal Epithelium | 0.14 | 0.92 | exon, intron | DDRGK1 | U |
| 2529 | chr14 | 99936328 | 99936718 | Breast Basal Epithelium | 0.16 | 0.94 | intron | SETD3 | U |
| 2530 | chr16 | 22336742 | 22337089 | Breast Basal Epithelium | 0.18 | 0.95 | intron | POLR35 | U |
| 2531 | chr18 | 20551897 | 20552303 | Breast Basal Epithelium | 0.17 | 0.94 | intron | RBBP8 | U |
| 2532 | chr1 | 28289721 | 28290024 | Breast Basal Epithelium | 0.17 | 0.93 | intron | XKR8 | U |
| 2533 | chr13 | 24873567 | 24873865 | Breast Basal Epithelium | 0.2 | 0.94 | intron | SPATA13 | U |
| 2534 | chr9 | 134350774 | 134351152 | Breast Basal Epithelium | 0.21 | 0.94 | exon | PRAC2B | U |
| 2535 | chr7 | 990481141 | 99048351 | Breast Basal Epithelium | 0.23 | 0.94 | intron | CPSF4 | U |
| 2536 | chr9 | 139102868 | 139103122 | Breast Basal Epithelium | 0.31 | 0.93 | intron | QSOX2 | U |
| 2537 | chr1 | 234529405 | 234529509 | Breast Basal Epithelium | 0.09 | 0.94 | exon | TARBP1 | U |
| 2538 | chr1 | 154557601 | 154557782 | Breast Basal Epithelium | 0.08 | 0.93 | intron | ADAR | U |
| 2539 | chr4 | 10083001 | 10083099 | Breast Basal Epithelium | 0.06 | 0.9 | intron | WDR1 | U |
| 2540 | chr17 | 78339277 | 78339500 | Breast Basal Epithelium | 0.1 | 0.93 | intron | ANF213 | U |
| 2541 | chr2 | 10712116 | 10712382 | Breast Basal Epithelium | 0.08 | 0.91 | exon | NOL10 | U |
| 2542 | chr19 | 40907571 | 4091050 | Breast Basal Epithelium | 0.1 | 0.93 | intron | MAP2K2 | U |
| 2543 | chr1 | 115768600 | 115768904 | Breast Basal Epithelium | 0.05 | 0.88 | Intergenic | NGF | U |
| 2544 | chr8 | 103755444 | 103755750 | Breast Basal Epithelium | 0.09 | 0.92 | Intergenic | KLF10 | U |
| 2545 | chr5 | 150649233 | 150649655 | Breast Basal Epithelium | 0.08 | 0.91 | exon | GM2A | U |
| 2546 | chr14 | 100138645 | 100138816 | Breast Basal Epithelium | 0.07 | 0.89 | exon | HHIPL1 | U |
| 2547 | chr19 | 14040781 | 14041147 | Breast Basal Epithelium | 0.05 | 0.87 | intron, TTS | CC2D1A, PODNL1 | U |
| 2548 | chr6 | 35056412 | 35056499 | Breast Basal Epithelium | 0.12 | 0.93 | exon | ANKS1A | U |
| 2549 | chr2 | 65299908 | 65300026 | Breast Basal Epithelium | 0.08 | 0.89 | exon | CEP68 | U |
| 2550 | chr16 | 58292241 | 58292423 | Breast Basal Epithelium | 0.1 | 0.91 | intron, exon | CCDC113, CCDC113 | U |
| 2551 | chr10 | 134239245 | 134239434 | Breast Basal Epithelium | 0.1 | 0.91 | Intergenic | C10orf91 | U |
| 2552 | chr2 | 25355689 | 25355881 | Breast Basal Epithelium | 0.13 | 0.94 | exon | EFR38 | U |
| 2553 | chr17 | 64439761 | 64440042 | Breast Basal Epithelium | 0.06 | 0.87 | intron | PRKCA | U |
| 2554 | chr3 | 8817175 | 8817507 | Breast Basal Epithelium | 0.07 | 0.88 | Intergenic | OXTR | U |
| 2555 | chr2 | 11303769 | 11304163 | Breast Basal Epithelium | 0.1 | 0.91 | intron | PQLC3 | U |
| 2556 | chr18 | 34966967 | 34967016 | Breast Basal Epithelium | 0.11 | 0.91 | intron | CELF4 | U |
| 2557 | chr9 | 139959096 | 139959161 | Breast Basal Epithelium | 0.09 | 0.89 | exon | SAPCD2 | U |
| 2558 | chr17 | 78262251 | 78262378 | Breast Basal Epithelium | 0.09 | 0.89 | intron | RNF213 | U |
| 2559 | chr6 | 1687692091 | 168769379 | Breast Basal Epithelium | 0.07 | 0.87 | Intergenic | DACT2 | U |
| 2560 | chr11 | 12031670 | 12031966 | Breast Basal Epithelium | 0.08 | 0.88 | promoter-TSS | DKK3 | U |
| 2561 | chr1 | 24945158 | 24945547 | Breast Basal Epithelium | 0.06 | 0.86 | Intergenic | SRRM1 | U |
| 2562 | chr2 | 10543898 | 10544302 | Breast Basal Epithelium | 0.11 | 0.91 | intron | HPCAL1 | U |
| 2563 | chr6 | 106580209 | 106580282 | Breast Basal Epithelium | 0.11 | 0.9 | Intergenic | PRDM1 | U |
| 2564 | chr15 | 68636277 | 68636456 | Breast Basal Epithelium | 0.06 | 0.85 | intron | ITGA11 | U |
| 2565 | chr8 | 713960541 | 71396292 | Breast Basal Epithelium | 0.11 | 0.9 | Intergenic | NCOA2 | U |
| 2566 | chr2 | 23645918 | 23646162 | Breast Basal Epithelium | 0.14 | 0.93 | intron | KLHL29 | U |
| 2567 | chr1 | 19583402 | 19583681 | Breast Basal Epithelium | 0.1 | 0.89 | intron | MRTO4 | U |
| 2568 | chr2 | 235347621 | 235348007 | Breast Basal Epithelium | 0.12 | 0.91 | Intergenic | ARLAC | U |
| 2569 | chr21 | 18266959 | 18267054 | Breast Basal Epithelium | 0.12 | 0.9 | Intergenic | MIR12582 | U |
| 2570 | chr16 | 81701854 | 81701982 | Breast Basal Epithelium | 0.13 | 0.91 | TTS, intron | LOC100129617, CMIP [symbol] | U |
| 2571 | chr15 | 75430416 | 75430565 | Breast Basal Epithelium | 0.11 | 0.89 | Intergenic | C15orf39 | U |
| 2572 | chr14 | 102713035 | 102713253 | Breast Basal Epithelium | 0.14 | 0.92 | intron | MOK | U |
| 2573 | chr19 | 412823721 | 41282679 | Breast Basal Epithelium | 0.12 | 0.9 | intron | MIA-RAB4B | U |
| 2574 | chr3 | 101372548 | 101372907 | Breast Basal Epithelium | 0.16 | 0.94 | intron | ZBTB11 | U |
| 2575 | chr7 | 501558 | 501946 | Breast Basal Epithelium | 0.12 | 0.9 | Intergenic | PDGFA | U |
| 2576 | chr1 | 112354156 | 112354259 | Breast Basal Epithelium | 0.11 | 0.88 | intron | KCND3 | U |
| 2577 | chr16 | 68876141 | 68876294 | Breast Basal Epithelium | 0.13 | 0.9 | Intergenic | TANGO6 | U |
| 2578 | chr10 | 127680637 | 127680967 | Breast Basal Epithelium | 0.1 | 0.87 | intron | FANK1 | U |
| 2579 | chr20 | 58148658 | 58148995 | Breast Basal Epithelium | 0.08 | 0.85 | Intergenic | LOC100506384 | U |
| 2580 | chr17 | 62016913 | 62017289 | Breast Basal Epithelium | 0.08 | 0.85 | exon | SCN4A | U |
| 2581 | chr3 | 188679687 | 188680097 | Breast Basal Epithelium | 0.16 | 0.93 | Intergenic | TPRG1 | U |
| 2582 | chr2 | 37002030 | 37002197 | Breast Basal Epithelium | 0.12 | 0.88 | intron | VIT | U |
| 2583 | chr2 | 85212332 | 85212545 | Breast Basal Epithelium | 0.15 | 0.91 | intron | KCMF1 | U |
| 2584 | chr1 | 214549650 | 214549865 | Breast Basal Epithelium | 0.17 | 0.93 | exon | PTPN14 | U |
| 2585 | chr1 | 249109855 | 249110124 | Breast Basal Epithelium | 0.14 | 0.9 | intron | SH38P5L | U |
| 2586 | chr12 | 19794155 | 19794500 | Breast Basal Epithelium | 0.07 | 0.83 | Intergenic | AEBP2 | U |
| 2587 | chr17 | 63075041 | 63075401 | Breast Basal Epithelium | 0.11 | 0.87 | Intergenic | GNA13 | U |
| 2588 | chr13 | 110986666 | 110986865 | Breast Basal Epithelium | 0.12 | 0.87 | intron | COL4A2 | U |
| 2589 | chr18 | 10350798 | 10350998 | Breast Basal Epithelium | 0.11 | 0.86 | Intergenic | APCDD1 | U |
| 2590 | chr16 | 1715962 | 1716165 | Breast Basal Epithelium | 0.18 | 0.93 | intron, exon | CRAMP1L, CRAMPIL | U |
| 2591 | chr1 | 2292590423 | 229259265 | Breast Basal Epithelium | 0.14 | 0.89 | Intergenic | RAB4A | U |
| 2592 | chr16 | 25087469 | 25087707 | Breast Basal Epithelium | 0.16 | 0.9 | Intergenic | LCMT1 | U |
| 2593 | chr1 | 117278031 | 117278177 | Breast Basal Epithelium | 0.18 | 0.91 | Intergenic | CD2 | U |
| 2594 | chr19 | 2556720 | 2556918 | Breast Basal Epithelium | 0.16 | 0.89 | intron | GNG7 | U |
| 2595 | chr17 | 21111328 | 21111601 | Breast Basal Epithelium | 0.19 | 0.92 | intron | TMEM11 | U |
| 2596 | chr1 | 17897130 | 17897417 | Breast Basal Epithelium | 0.19 | 0.92 | intron | ARHGEF10L | U |
| 2597 | chr17 | 55365004 | 55365310 | Breast Basal Epithelium | 0.18 | 0.91 | intron | MSI2 | U |
| 2598 | chr2 | 219041675 | 219042030 | Breast Basal Epithelium | 0.23 | 0.93 | Intergenic | CXCR1 | U |
| 2599 | chr15 | 53920307 | 63920521 | Breast Basal Epithelium | 0.19 | 0.91 | intron | HERC1 | U |
| 2600 | chr19 | 56596817 | 56597040 | Breast Basal Epithelium | 0.17 | 0.89 | Intergenic | ZNF787 | U |
| 2601 | chr12 | 114275299 | 114275615 | Breast Basal Epithelium | 0.2 | 0.92 | intron | RBM19 | U |
| 2602 | chr16 | 4947620 | 4947983 | Breast Basal Epithelium | 0.17 | 0.89 | intron | PPL | U |
| 2603 | chr17 | 45992349 | 45992757 | Breast Basal Epithelium | 0.2 | 0.92 | intron | SP2 | U |
| 2604 | chr16 | 23573777 | 23574206 | Breast Basal Epithelium | 0.21 | 0.92 | intron, exon | UBFD1, UBFD1 | U |
| 2605 | chr17 | 78157897 | 78158060 | Breast Basal Epithelium | 0.21 | 0.91 | exon | CARD14 | U |
| 2606 | chr12 | 113822938 | 113823115 | Breast Basal Epithelium | 0.24 | 0.94 | exor | PLBD2 | U |
| 2607 | chr1 | 229370733 | 229370890 | Breast Basal Epithelium | 0.22 | 0.9 | Intergenic | RAB4A | U |
| 2608 | chr17 | 21106166 | 21106451 | Breast Basal Epithelium | 0.2 | 0.88 | intron | TMEM11 | U |
| 2609 | chr5 | 169576283 | 169576577 | Breast Basal Epithelium | 0.21 | 0.89 | Intergenic | FOXI1 | U |
| 2610 | chr12 | 12387113 | 12387379 | Breast Basal Epithelium | 0.29 | 0.95 | intron | LRP6 | U |
| 2611 | chr21 | 46397434 | 46397804 | Breast Basal Epithelium | 0.29 | 0.95 | TTS | FAM207A | U |
| 2612 | chr19 | 47491676 | 47492048 | Breast Basal Epithelium | 0.16 | 0.8 | intron | ARHGAP35 | U |
| 2613 | chr1 | 6008175 | 6008578 | Breast Basal Epithelium | 0.29 | 0.92 | intron | NPHP4 | U |
| 2614 | chr2 | 109076333 | 109076747 | Breast Basal Epithelium | 0.3 | 0.93 | intron | GCC2 | U |
| 2615 | chr15 | 64434782 | 64435204 | Breast Basal Epithelium | 0.28 | 0.9 | fexon | SNX1 | U |
| 2616 | chr16 | 798881 | 799060 | Breast Basal Epithelium | 0.32 | 0.93 | Intergenic | NARFL | U |
| 2617 | chr9 | 139103335 | 139103781 | Breast Basal Epithelium | 0.04 | 0.92 | intron | QSOX2 | U |
| 2618 | chr22 | 18277229 | 18277535 | Breast Basal Epithelium | 0.1 | 0.93 | intron | MICAL3 | U |
| 2619 | chr22 | 36692812 | 36693194 | Breast Basal Epithelium | 0.1 | 0.93 | intron | MYH9 | U |
| 2620 | chr20 | 39803942 | 39804431 | Breast Basal Epithelium | 0.1 | 0.93 | TTS, exon | PLCG1 | U |
| 2621 | chr19 | 14041071 | 14041305 | Breast Basal Epithelium | 0.12 | 0.94 | TTS | PODNL1 | U |
| 2622 | chr22 | 39822758 | 39823142 | Breast Basal Epithelium | 0.19 | 0.93 | intron | TAB1 | U |
| 2623 | chr5 | 176919342 | 176919703 | Breast Basal Epithelium | 0.22 | 0.95 | intron | PDLIM7 | U |
| 2624 | chr19 | 4944306 | 4944584 | Breast Basal Epithelium | 0.24 | 0.96 | intron | UHRF1 | U |
| 2625 | chr5 | 1808108 | 1808451 | Breast Basal Epithelium | 0.28 | 0.94 | intron | NDUFS6 | U |
| 2626 | chr13 | 113887317 | 113887803 | Breast Basal Epithelium | 0.07 | 0.94 | exon | CUL4A | U |
| 2627 | chr20 | 33925690 | 33925818 | Breast Basal Epithelium | 0.04 | 0.9 | intron | UQCC | U |
| 2628 | chr20 | 31015007 | 31015485 | Breast Basal Epithelium | 0.07 | 0.93 | intron | ASXL1 | U |
| 2629 | chr20 | 62314803 | 62315091 | Breast Basal Epithelium | 0.13 | 0.93 | intron | RTEL1-TNFRSF6B | U |
| 2630 | chr3 | 41287899 | 41288395 | Breast Basal Epithelium | 0.08 | 0.9 | TTS | ULK4 | U |
| 2631 | chr19 | 14041194 | 14041306 | Breast Basal Epithelium | 0.14 | 0.94 | TTS | PODNL1 | U |
| 2632 | chr9 | 72024669 | 72024813 | Breast Basal Epithelium | 0.08 | 0.88 | Intergenic | FAM189A2 | U |
| 2633 | chr11 | 3164799 | 3164941 | Breast Basal Epithelium | 0.11 | 0.9 | intron | OSBPL5 | U |
| 2634 | chr21 | 46398140 | 46398323 | Breast Basal Epithelium | 0.17 | 0.96 | Intergenic | LINC00163 | U |
| 2635 | chr21 | 46424553 | 46424669 | Breast Basal Epithelium | 0.13 | 0.91 | exon, promoter-TSS | LINC00162, LINC00161U | U |
| 2636 | chr7 | 531331 | 531628 | Breast Basal Epithelium | 0.12 | 0.9 | Intergenic | PDGFA | U |
| 2637 | chr19 | 4944196 | 4944585 | Breast Basal Epithelium | 0.18 | 0.95 | intron | UHRF1 | U |
| 2638 | chr21 | 46424069 | 46424480 | Breast Basal Epithelium | 0.12 | 0.88 | intron | LINC00162 | U |
| 2639 | chr19 | 39914132 | 39914303 | Breast Basal Epithelium | 0.16 | 0.91 | exon, intron | PLEKHG2, PLEKHG2 | U |
| 2640 | chr19 | 11537720 | 11537908 | Breast Basal Epithelium | 0.18 | 0.93 | exon, intron | CCDC151, CCDC151 | U |
| 2641 | chr2 | 2410678501 | 241068039 | Breast Basal Epithelium | 0.14 | 0.89 | intron | MYEOV2 | U |
| 2642 | chr5 | 139725 | 139937 | Breast Basal Epithelium | 0.13 | 0.86 | promoter-TSS | PLEKHG48 | U |
| 2643 | chr7 | 100403023 | 100403289 | Breast Basal Epithelium | 0.19 | 0.92 | exon | EPHB4 | U |
| 2644 | chr9 | 98206494 | 98206826 | Breast Basal Epithelium | 0.16 | 0.89 | exon | PTCH1 | U |
| 2645 | chr19 | 14041313 | 14041483 | Breast Basal Epithelium | 0.13 | 0.83 | TTS | PODNL1 | U |
| 2646 | chr10 | 135085004 | 135085220 | Breast Basal Epithelium | 0.21 | 0.91 | exon | ADAM8 | U |
| 2647 | chr22 | 31483907 | 31484125 | Breast Basal Epithelium | 0.25 | 0.94 | intron | SMTN | U |
| 2648 | chr8 | 1288674883 | 128867655 | Breast Basal Epithelium | 0.22 | 0.88 | Intergenic | PVT1 | U |
| 2649 | chr16 | 3170204 | 3170619 | Breast Basal Epithelium | 0.28 | 0.91 | exon, TTS | ZNF205, ZNF205 | U |
| 2650 | chr11 | 57069591 | 57070017 | Breast Basal Epithelium | 0.32 | 0.95 | intron | TNKS1BP1 | U |
| 2651 | chr22 | 40804348 | 40804641 | Breast Basal Epithelium | 0.31 | 0.88 | intron | SGSM3 | U |
| 2652 | chr22 | 22668568 | 22668874 | Breast Basal Epithelium | 0.87 | 0.07 | Intergenic | ZBTB40 | M |
| 2653 | chr10 | 123804329 | 123804438 | Breast Basal Epithelium | 0.89 | 0.14 | intron | TACC2 | M |
| 2654 | chr6 | 169652865 | 169653336 | Breast Basal Epithelium | 0.85 | 0.12 | intron | THBS2 | M |
| 2655 | chr17 | 70515212 | 70515555 | Breast Basal Epithelium | 0.91 | 0.2 | intron | LINC00673 | M |
| 2656 | chr12 | 54372756 | 54373041 | Breast Basal Epithelium | 0.78 | 0.13 | Intergenic | HOXC11 | M |
| 2657 | chr20 | 55646391 | 55646805 | Breast Basal Epithelium | 0.7 | 0.06 | Intergenic | BMP7 | M |
| 2658 | chr5 | 134824947 | 134825056 | Breast Basal Epithelium | 0.69 | 0.07 | Intergenic | TIFAB | M |
| 2659 | chr17 | 40933276 | 40933355 | Breast Basal Epithelium | 0.77 | 0.23 | exon, intron | WNK4, WNK4 | M |
| 2660 | chr12 | 112205077 | 112205245 | Breast Basal Epithelium | 0.91 | 0.04 | intron | ALDH2 | M |
| 2661 | chr1 | 9600250 | 9600444 | Breast Basal Epithelium | 0.85 | 0.05 | intron | SLC25A33 | M |
| 2662 | chr12 | 54090151 | 54090388 | Breast Basal Epithelium | 0.85 | 0.05 | Intergenic | ATP5G2 | M |
| 2663 | chr5 | 3590802 | 3591183 | Breast Basal Epithelium | 0.85 | 0.06 | Intergenic | IRX1 | M |
| 2664 | chr13 | 37004536 | 37004748 | Breast Basal Epithelium | 0.9 | 0.14 | Intergenic | CCNA1 | M |
| 2665 | chr4 | 113442655 | 113442919 | Breast Basal Epithelium | 0.87 | 0.12 | Intergenic | NEUROG2 | M |
| 2666 | chr5 | 1395253873 | 139525796 | Breast Basal Epithelium | 0.78 | 0.03 | Intergenic | IGIP | M |
| 2667 | chr5 | 35905903 | 3590784 | Breast Basal Epithelium | 0.84 | 0.1 | intergenic | IRX1 | M |
| 2668 | chr22 | 41810435 | 41810666 | Breast Basal Epithelium | 0.81 | 0.09 | Intergenic, Intergeni [symbol] | TEF, TOB2 | M |
| 2669 | chr13 | 37004787 | 37005108 | Breast Basal Epithelium | 0.8 | 0.08 | Intergenic | CCNA1 | M |
| 2670 | chr14 | 91883165 | 91883566 | Breast Basal Epithelium | 0.77 | 0.09 | intron | CCDC88C | M |
| 2671 | chr5 | 174120383 | 174120663 | Breast Basal Epithelium | 0.81 | 0.14 | Intergenic | MSX2 | M |
| 2672 | chr5 | 134370245 | 134370428 | Breast Basal Epithelium | 0.72 | 0.09 | promoter-TSS | PITX1 | M |
| 2673 | chr2 | 2417607781 | 241761040 | Breast Basal Epithelium | 0.73 | 0.1 | Intergenic | KIF1A | M |
| 2674 | chr17 | 1131684 | 1132005 | Breast Basal Epithelium | 0.61 | 0.05 | Intergenic | BHLHA9 | M |
| 2675 | chr2 | 119602770 | 119603073 | Breast Basal Epithelium | 0.64 | 0.1 | intron | EN1 | M |
| 2676 | chr16 | 84402414 | 84402587 | Breast Basal Epithelium | 0.58 | 0.09 | promoter-TSS, intro [symbol] | ATP2C2, ATP2C2 | M |
| 2677 | chr6 | 83921378 | 83921763 | Breast Luminal Epithelium | 0.02 | 0.91 | exon | ME1 | U |
| 2678 | chr10 | 61931723 | 61932133 | Breast Luminal Epithelium | 0.05 | 0.92 | intron | ANK3 | U |
| 2679 | chr8 | 8572296 | 8572474 | Breast Luminal Epithelium | 0.03 | 0.87 | Intergenic | CLDN23 | U |
| 2680 | chr2 | 2364241171 | 236424362 | Breast Luminal Epithelium | 0.04 | 0.88 | intron | AGAP1 | U |
| 2681 | chr1 | 15281037 | 15281104 | Breast Luminal Epithelium | 0.06 | 0.89 | intron | KAZN | U |
| 2682 | chr21 | 44850180 | 44850272 | Breast Luminal Epithelium | 0.09 | 0.88 | Intergenic | SIK1 | U |
| 2683 | chr17 | 1848936 | 1849265 | Breast Luminal Epithelium | 0.15 | 0.93 | intron | RTN4RL1 | U |
| 2684 | chr18 | 73395621 | 73395760 | Breast Luminal Epithelium | 0.12 | 0.89 | Intergenic | C18orf62 | U |
| 2685 | chr8 | 117231799 | 117232275 | Breast Luminal Epithelium | 0.14 | 0.89 | intron | LINC00536 | U |
| 2686 | chr12 | 5747733 | 5747897 | Breast Luminal Epithelium | 0.18 | 0.91 | intron | ANO2 | U |
| 2687 | chr9 | 139984622 | 139985009 | Breast Luminal Epithelium | 0.3 | 0.9 | intron | MAN1B1 | U |
| 2688 | chr3 | 193996432 | 193996676 | Breast Luminal Epithelium | 0.35 | 0.9 | Intergenic | LOC100131551 | U |
| 2689 | chr1 | 168067805 | 168068085 | Breast Luminal Epithelium | 0.01 | 0.89 | intron | GPR161 | U |
| 2690 | chr2 | 2406914801 | 240691613 | Breast Luminal Epithelium | 0.04 | 0.87 | intron | LOC150935 | U |
| 2691 | chr9 | 1391217101 | 139121865 | Breast Luminal Epithelium | 0.05 | 0.87 | intron | QSOX2 | U |
| 2692 | chr9 | 95596773 | 9560036 | Breast Luminal Epithelium | 0.05 | 0.87 | intron | PTPRD | U |
| 2693 | chr16 | 88017646 | 88017848 | Breast Luminal Epithelium | 0.06 | 0.86 | intron, exon | BANP, BANP | U |
| 2694 | chr19 | 283342 | 283701 | Breast Luminal Epithelium | 0.02 | 0.82 | intron | PPAP2C | U |
| 2695 | chr18 | 14798182 | 14798393 | Breast Luminal Epithelium | 0.06 | 0.85 | intron | ANKRD308 | U |
| 2696 | chr1 | 56392964 | 56393055 | Breast Luminal Epithelium | 0.13 | 0.91 | Intergenic | PPAP2B | U |
| 2697 | chr11 | 64993593 | 6499714 | Breast Luminal Epithelium | 0.07 | 0.85 | intron | ARFIP2 | U |
| 2698 | chr5 | 149880893 | 149881075 | Breast Luminal Epithelium | 0.12 | 0.88 | Intergenic | NDST1 | U |
| 2699 | chr17 | 64658256 | 64658540 | Breast Luminal Epithelium | 0.16 | 0.92 | intron | PRKCA | U |
| 2700 | chr13 | 100738533 | 100738798 | Breast Luminal Epithelium | 0.12 | 0.87 | Intergenic | PCCA | U |
| 2701 | chr6 | 42397428 | 42397763 | Breast Luminal Epithelium | 0.14 | 0.89 | intron | TRERF1 | U |
| 2702 | chr19 | 36607845 | 36608146 | Breast Luminal Epithelium | 0.07 | 0.8 | intron | TBCB | U |
| 2703 | chr1 | 41888473 | 41888534 | Breast Luminal Epithelium | 0.18 | 0.89 | Intergenic | EDN2 | U |
| 2704 | chr21 | 31587857 | 31588020 | Breast Luminal Epithelium | 0.13 | 0.84 | exon | CLDN8 | U |
| 2705 | chr6 | 143144273 | 143144490 | Breast Luminal Epithelium | 0.18 | 0.89 | intron | HIVEP2 | U |
| 2706 | chr11 | 1103180963 | 110318482 | Breast Luminal Epithelium | 0.2 | 0.91 | intron | FDX1 | U |
| 2707 | chr20 | 50278024 | 60278107 | Breast Luminal Epithelium | 0.14 | 0.84 | intron | CDH4 | U |
| 2708 | chr4 | 186165406 | 186165606 | Breast Luminal Epithelium | 0.16 | 0.86 | intron | SNX25 | U |
| 2709 | chr2 | 75824876 | 75825203 | Breast Luminal Epithelium | 0.22 | 0.92 | Intergenic | EVA1A | U |
| 2710 | chr2 | 237494458 | 237494786 | Breast Luminal Epithelium | 0.19 | 0.89 | Intergenic | CXCR7 | U |
| 2711 | chr1 | 42593479 | 42593638 | Breast Luminal Epithelium | 0.18 | 0.87 | Intergenic | GUCA2B | U |
| 2712 | chr15 | 48518739 | 48518922 | Breast Luminal Epithelium | 0.17 | 0.86 | intron | SLC12A1 | U |
| 2713 | chr9 | 132518514 | 132518900 | Breast Luminal Epithelium | 0.18 | 0.87 | Intergenic | PTGES | U |
| 2714 | chr21 | 40307327 | 40307556 | Breast Luminal Epithelium | 0.16 | 0.84 | Intergenic | ETS2 | U |
| 2715 | chr16 | 4690375 | 4590492 | Breast Luminal Epithelium | 0.1 | 0.77 | intron | MGRN1 | U |
| 2716 | chr11 | 71324397 | 71324560 | Breast Luminal Epithelium | 0.17 | 0.84 | Intergenic | KRTAP5-11 | U |
| 2717 | chr13 | 30492545 | 30492843 | Breast Luminal Epithelium | 0.24 | 0.91 | Intergenic | LINC00544 | U |
| 2718 | chr10 | 29566189 | 29566527 | Breast Luminal Epithelium | 0.19 | 0.86 | Intergenic | LYZL1 | U |
| 2719 | chr22 | 43273432 | 43273572 | Breast Luminal Epithelium | 0.23 | 0.89 | intron | PACSIN2 | U |
| 2720 | chr3 | 41835526 | 41836013 | Breast Luminal Epithelium | 0.18 | 0.84 | intron | ULK4 | U |
| 2721 | chr3 | 53837301 | 53837412 | Breast Luminal Epithelium | 0.25 | 0.9 | intron | CACNA1D | U |
| 2722 | chr1 | 110659458 | 110659634 | Breast Luminal Epithelium | 0.2 | 0.85 | Intergenic | UBL4B | U |
| 2723 | chr15 | 79483422 | 79483667 | Breast Luminal Epithelium | 0.22 | 0.87 | TTS | LOC729911 | U |
| 2724 | chr14 | 1005137543 | 100514036 | Breast Luminal Epithelium | 0.26 | 0.91 | Intergenic | EVL | U |
| 2725 | chr8 | 37127074 | 37127224 | Breast Luminal Epithelium | 0.24 | 0.87 | Intergenic | ZNF703 | U |
| 2726 | chr7 | 149372167 | 149372417 | Breast Luminal Epithelium | 0.19 | 0.82 | Intergenic | KRBA1 | U |
| 2727 | chr14 | 105495862 | 105496129 | Breast Luminal Epithelium | 0.22 | 0.85 | Intergenic | CDCA4 | U |
| 2728 | chr12 | 92685704 | 92686195 | Breast Luminal Epithelium | 0.26 | 0.89 | Intergenic | CLLU1 | U |
| 2729 | chr6 | 37727275 | 37727350 | Breast Luminal Epithelium | 0.24 | 0.86 | Intergenic | ZFAND3 | U |
| 2730 | chr11 | 65096709 | 65096872 | Breast Luminal Epithelium | 0.23 | 0.85 | Intergenic | DPF2 | U |
| 2731 | chr16 | 514377 | 514552 | Breast Luminal Epithelium | 0.2 | 0.8 | intron | RAB11FIP3 | U |
| 2732 | chr1 | 36993817 | 36993998 | Breast Luminal Epithelium | 0.26 | 0.86 | Intergenic | CSF3R | U |
| 2733 | chr18 | 55787062 | 55787428 | Breast Luminal Epithelium | 0.27 | 0.87 | intron | NEDD4L | U |
| 2734 | chr14 | 1056506403 | 105651010 | Breast Luminal Epithelium | 0.18 | 0.78 | Intergenic | NUDT14 | U |
| 2735 | chr6 | 33700074 | 33700413 | Breast Luminal Epithelium | 0.22 | 0.8 | intron | IPEK3 | U |
| 2736 | chr7 | 48827791 | 48828132 | Breast Luminal Epithelium | 0.29 | 0.87 | Intergenic | CDC14C | U |
| 2737 | chr15 | 73962832 | 73963178 | Breast Luminal Epithelium | 0.18 | 0.76 | Intergenic | CD276 | U |
| 2738 | chr2 | 47532143 | 47532560 | Breast Luminal Epithelium | 0.31 | 0.89 | Intergenic | EPCAM | U |
| 2739 | chr16 | 85753082 | 85753582 | Breast Luminal Epithelium | 0.29 | 0.87 | intron | C16orf74 | U |
| 2740 | chr6 | 33698746 | 33698979 | Breast Luminal Epithelium | 0.27 | 0.83 | intron | IP6K3 | U |
| 2741 | chr1 | 17958862 | 17958885 | Breast Luminal Epithelium | 0.34 | 0.89 | exon | ARHGEF10L | U |
| 2742 | chr11 | 6704333 | 6704356 | Breast Luminal Epithelium | 0.27 | 0.82 | intron | ADRBK1 | U |
| 2743 | chr6 | 34506082 | 34506318 | Breast Luminal Epithelium | 0.37 | 0.91 | exon | SPDEF | U |
| 2744 | chr1 | 231685353 | 231685834 | Breast Luminal Epithelium | 0.27 | 0.8 | intron | TSNAX-DISC1 | U |
| 2745 | chr8 | 11775427 | 11775639 | Breast Luminal Epithelium | 0.31 | 0.83 | Intergenic | CTSB | U |
| 2746 | chr7 | 45753641 | 45753810 | Breast Luminal Epithelium | 0.31 | 0.81 | exon | ADCY1 | U |
| 2747 | chr2 | 236787014 | 236787185 | Breast Luminal Epithelium | 0.31 | 0.81 | intron | AGAP1 | U |
| 2748 | chr15 | 29335159 | 29335363 | Breast Luminal Epithelium | 0.31 | 0.81 | intron | APBA2 | U |
| 2749 | chr12 | 15368653 | 15368873 | Breast Luminal Epithelium | 0.28 | 0.91 | intron | RERG | U |
| 2750 | chr19 | 10295669 | 10295839 | Breast Luminal Epithelium | 0.01 | 0.87 | intron | DNMT1 | U |
| 2751 | chr2 | 27433191 | 27433455 | Breast Luminal Epithelium | 0.09 | 0.92 | intron | SLC5A6 | U |
| 2752 | chr17 | 1523792 | 1523909 | Breast Luminal Epithelium | 0.1 | 0.89 | intron | SLC43A2 | U |
| 2753 | chr6 | 43748428 | 43748602 | Breast Luminal Epithelium | 0.11 | 0.9 | exon | VEGFA | U |
| 2754 | chr2 | 233986730 | 233986988 | Breast Luminal Epithelium | 0.04 | 0.81 | exon, intron | INPP5D | U |
| 2755 | chr1 | 204535920 | 204536232 | Breast Luminal Epithelium | 0.14 | 0.89 | Intergenic | MDM4 | U |
| 2756 | chr10 | 22047483 | 22047621 | Breast Luminal Epithelium | 0.19 | 0.93 | intron | DNAJC1 | U |
| 2757 | chr2 | 61471508 | 61471743 | Breast Luminal Epithelium | 0.2 | 0.94 | intron | USP34 | U |
| 2758 | chr19 | 40828736 | 40829046 | Breast Luminal Epithelium | 0.19 | 0.92 | intron | C19orf47 | U |
| 2759 | chr8 | 140755399 | 140755536 | Breast Luminal Epithelium | 0.16 | 0.88 | intron | TRAPPC9 | U |
| 2760 | chr21 | 46935507 | 46935981 | Breast Luminal Epithelium | 0.29 | 0.93 | exon | SLC19A1 | U |
| 2761 | chr14 | 105181998 | 105182490 | Breast Luminal Epithelium | 0.27 | 0.9 | intron | INF2 | U |
| 2762 | chr22 | 30190746 | 30191205 | Breast Luminal Epithelium | 0.31 | 0.94 | intron | ASCC2 | U |
| 2763 | chr7 | 6188781 | 6188836 | Breast Luminal Epithelium | 0.04 | 0.92 | intron | USP42 | U |
| 2764 | chr19 | 10295203 | 10295312 | Breast Luminal Epithelium | 0.02 | 0.89 | intron | DNMT1 | U |
| 2765 | chr19 | 1167117 | 1167262 | Breast Luminal Epithelium | 0.04 | 0.9 | intron | SBNO2 | U |
| 2766 | chr2 | 9776070 | 9776404 | Breast Luminal Epithelium | 0.04 | 0.88 | Intergenic | YWHAQ | U |
| 2767 | chr1 | 21573375 | 21573594 | Breast Luminal Epithelium | 0.06 | 0.89 | intron | ECE1 | U |
| 2768 | chr7 | 6188255 | 6188724 | Breast Luminal Epithelium | 0.07 | 0.9 | intron | USP42 | U |
| 2769 | chr17 | 30348093 | 30348317 | Breast Luminal Epithelium | 0.05 | 0.87 | promoter-TSS, exon | LRRC37B, LRRC37B | U |
| 2770 | chr4 | 57177980 | 57178217 | Breast Luminal Epithelium | 0.04 | 0.86 | intron | KIAA1211 | U |
| 2771 | chr10 | 104383917 | 104384312 | Breast Luminal Epithelium | 0.04 | 0.86 | intron | SUFU | U |
| 2772 | chr4 | 3217013 | 3217297 | Breast Luminal Epithelium | 0.08 | 0.87 | intron | HTT | U |
| 2773 | chr6 | 169641078 | 169641365 | Breast Luminal Epithelium | 0.08 | 0.87 | intron | THBS2 | U |
| 2774 | chr5 | 177525862 | 177525915 | Breast Luminal Epithelium | 0.09 | 0.87 | Intergenic | N4BP3 | U |
| 2775 | chr2 | 10600695 | 10600769 | Breast Luminal Epithelium | 0.08 | 0.84 | Intergenic | ODC1 | U |
| 2776 | chr11 | 75167903 | 75168178 | Breast Luminal Epithelium | 0.14 | 0.89 | intron | GDPD5 | U |
| 2777 | chr22 | 27859205 | 27859395 | Breast Luminal Epithelium | 0.09 | 0.83 | Intergenic | MN1 | U |
| 2778 | chr22 | 20014184 | 20014532 | Breast Luminal Epithelium | 0.15 | 0.86 | intron | TANGO2 | U |
| 2779 | chr11 | 3145253 | 3145385 | Breast Luminal Epithelium | 0.19 | 0.89 | intron | OSBPL5 | U |
| 2780 | chr22 | 46888626 | 46888921 | Breast Luminal Epithelium | 0.2 | 0.89 | intron | CELSR1 | U |
| 2781 | chr5 | 3630908 | 3631256 | Breast Luminal Epithelium | 0.17 | 0.86 | Intergenic | IRX1 | U |
| 2782 | chr8 | 1407703673 | 140770523 | Breast Luminal Epithelium | 0.19 | 0.87 | intron | TRAPPC9 | U |
| 2783 | chr14 | 35544037 | 35544208 | Breast Luminal Epithelium | 0.22 | 0.9 | intron | FAM177A1 | U |
| 2784 | chr11 | 70016959 | 70017320 | Breast Luminal Epithelium | 0.19 | 0.87 | exon | ANO1 | U |
| 2785 | chr16 | 15129301 | 15129420 | Breast Luminal Epithelium | 0.25 | 0.92 | intron, exon | PDXDC1, PDXDC1 | U |
| 2786 | chr10 | 12707807 | 12707930 | Breast Luminal Epithelium | 0.18 | 0.85 | intron | CAMK1D | U |
| 2787 | chr10 | 22047181 | 22047392 | Breast Luminal Epithelium | 0.23 | 0.88 | intron | DNAJC1 | U |
| 2788 | chr16 | 80641327 | 80641624 | Breast Luminal Epithelium | 0.25 | 0.9 | intron | CDYL2 | U |
| 2789 | chr19 | 4990593 | 4990775 | Breast Luminal Epithelium | 0.26 | 0.88 | intron | KDM4B | U |
| 2790 | chr14 | 56607984 | 56608366 | Breast Luminal Epithelium | 0.28 | 0.9 | intron | PELI2 | U |
| 2791 | chr9 | 1339678153 | 133968043 | Breast Luminal Epithelium | 0.24 | 0.85 | exon | LAMC3 | U |
| 2792 | chr6 | 20106040 | 20106421 | Breast Luminal Epithelium | 0.29 | 0.9 | intron | MBOAT1 | U |
| 2793 | chr4 | 184343198 | 184343390 | Breast Luminal Epithelium | 0.31 | 0.9 | Intergenic | CDKN2AIP | U |
| 2794 | chr6 | 34506937 | 34507148 | Breast Luminal Epithelium | 0.3 | 0.89 | exon | SPDEF | U |
| 2795 | chr10 | 29414135 | 29414418 | Breast Luminal Epithelium | 0.28 | 0.87 | Intergenic | LYZLI | U |
| 2796 | chr22 | 44564263 | 44564384 | Breast Luminal Epithelium | 0.34 | 0.91 | intron | PARVB | U |
| 2797 | chr8 | 141054097 | 141054499 | Breast Luminal Epithelium | 0.31 | 0.87 | intron | TRAPPC9 | U |
| 2798 | chr9 | 96105123 | 96105421 | Breast Luminal Epithelium | 0.31 | 0.86 | intron | C9orf129 | U |
| 2799 | chr12 | 111402971 | 111403283 | Breast Luminal Epithelium | 0.26 | 0.8 | Intergenic | LOC100131138 | U |
| 2800 | chr3 | 46949042 | 46949419 | Breast Luminal Epithelium | 0.32 | 0.86 | Intergenic | PTH1R | U |
| 2801 | chr20 | 30530332 | 30530753 | Breast Luminal Epithelium | 0.35 | 0.87 | intron, TTS | TTLL9, TTLL9 | U |
| 2802 | chr17 | 45946181 | 45946406 | Breast Luminal Epithelium | 0.31 | 0.78 | Intergenic | SP6 | U |
| 2803 | chr12 | 54433551 | 54433688 | Breast Luminal Epithelium | 0.87 | 0.12 | intron | HOXC4 | M |
| 2804 | chr3 | 180462104 | 180462290 | Breast Luminal Epithelium | 0.88 | 0.2 | Intergenic | CCDC39 | M |
| 2805 | chr1 | 89663866 | 89664034 | Breast Luminal Epithelium | 0.75 | 0.13 | intron | GBP4 | M |
| 2806 | chr16 | 629196 | 629406 | Breast Luminal Epithelium | 0.81 | 0.19 | exon, intron | PIGQ, PIGQ | M |
| 2807 | chr9 | 129400487 | 129400618 | Breast Luminal Epithelium | 0.76 | 0.2 | intron | LMX1B | M |
| 2808 | chr1 | 27961103 | 27961345 | Breast Luminal Epithelium | 0.72 | 0.17 | intron | FGR | M |
| 2809 | chr12 | 99287010 | 99287145 | Breast Luminal Epithelium | 0.69 | 0.2 | intron | ANKS1B | M |
| 2810 | chr19 | 47920660 | 47920904 | Breast Luminal Epithelium | 0.6 | 0.12 | intron | MEIS3 | M |
| 2811 | chr5 | 16600590 | 16600747 | Breast Luminal Epithelium | 0.65 | 0.19 | intron | FAM134B | M |
| 2812 | chr5 | 43040044 | 43040376 | Breast Luminal Epithelium | 0.57 | 0.11 | exon | ANXA2R | M |
| 2813 | chr1 | 18526236 | 18526402 | Breast Luminal Epithelium | 0.68 | 0.28 | intron | IGSF21 | M |
| 2814 | chrX | 1050652911 | 105065501 | Breast Luminal Epithelium | 0.67 | 0.29 | Intergenic | NRK | M |
| 2815 | chrX | 21676769 | 21676820 | Breast Luminal Epithelium | 0.63 | 0.28 | promoter-TSS | KLHL34 | M |
| 2816 | chr6 | 103246276 | 103246718 | Breast Luminal Epithelium | 0.64 | 0.1 | Intergenic | GRIK2 | M |
| 2817 | chr11 | 61062962 | 61063044 | Breast Luminal Epithelium | 0.9 | 0.12 | promoter-TSS | VWCE | M |
| 2818 | chr6 | 50818692 | 50819121 | Breast Luminal Epithelium | 0.87 | 0.1 | Intergenic | TFAP2B | M |
| 2819 | chr12 | 54408233 | 54408284 | Breast Luminal Epithelium | 0.84 | 0.11 | Intergenic | HOXC6 | M |
| 2820 | chr3 | 127794984 | 127795235 | Breast Luminal Epithelium | 0.81 | 0.11 | Intergenic | SEC61A1 | M |
| 2821 | chr9 | 129445271 | 129445732 | Breast Luminal Epithelium | 0.66 | 0.03 | intron | LMX1B | M |
| 2822 | chr15 | 736596871 | 73659812 | Breast Luminal Epithelium | 0.73 | 0.18 | exon, intron | HCN4, HCN4 | M |
| 2823 | chr17 | 60885306 | 60885424 | Breast Luminal Epithelium | 0.74 | 0.2 | intron | MARCH10 | M |
| 2824 | chr9 | 129445737 | 129445865 | Breast Luminal Epithelium | 0.6 | 0.07 | intron | LMX1B | M |
| 2825 | chr5 | 1741474443 | 174147569 | Breast Luminal Epithelium | 0.61 | 0.12 | Intergenic | MSX2 | M |
| 2826 | chr9 | 109622286 | 109622383 | Breast Luminal Epithelium | 0.55 | 0.15 | Intergenic | ZNF462 | M |
| 2827 | chr19 | 49466763 | 49467029 | Breast Luminal Epithelium | 0.53 | 0.13 | Intergenic | FTL | M |
| 2828 | chr16 | 22363430 | 22363834 | Lung Alveolar Epithelium | 0.05 | 0.95 | intron | CDR2 | U |
| 2829 | chr19 | 33202756 | 33203019 | Lung Alveolar Epithelium | 0.07 | 0.93 | exon | NUDT19 | U |
| 2830 | chr15 | 57626002 | 57626444 | Lung Alveolar Epithelium | 0.06 | 0.91 | Intergenic | LOC283663 | U |
| 2831 | chr22 | 46109795 | 46110257 | Lung Alveolar Epithelium | 0.08 | 0.93 | intron | ATXN10 | U |
| 2832 | chr3 | 182870189 | 182870399 | Lung Alveolar Epithelium | 0.04 | 0.89 | intron | LAMP3 | U |
| 2833 | chr2 | 100131233 | 100131368 | Lung Alveolar Epithelium | 0.08 | 0.9 | Intergenic | REV1 | U |
| 2834 | chr1 | 54554193 | 54554672 | Lung Alveolar Epithelium | 0.1 | 0.92 | exon | TCEANC2 | U |
| 2835 | chr14 | 96790748 | 96790914 | Lung Alveolar Epithelium | 0.13 | 0.95 | intron | ATG2B | U |
| 2836 | chr17 | 37700769 | 37701018 | Lung Alveolar Epithelium | 0.13 | 0.95 | Intergenic | NEUROD2 | U |
| 2837 | chr7 | 66074950 | 66075088 | Lung Alveolar Epithelium | 0.11 | 0.92 | Intergenic | KCTD7 | U |
| 2838 | chr17 | 55071173 | 55071656 | Lung Alveolar Epithelium | 0.15 | 0.93 | intron | SCPEP1 | U |
| 2839 | chr4 | 129396222 | 129396578 | Lung Alveolar Epithelium | 0.03 | 0.92 | Intergenic | PGRMC2 | U |
| 2840 | chr4 | 103637380 | 103637623 | Lung Alveolar Epithelium | 0.06 | 0.93 | intron | MANBA | U |
| 2841 | chr11 | 68510996 | 68511288 | Lung Alveolar Epithelium | 0.04 | 0.91 | intron | MTL5 | U |
| 2842 | chr17 | 53293250 | 53293655 | Lung Alveolar Epithelium | 0.03 | 0.9 | Intergenic | HLF | U |
| 2843 | chr5 | 132720735 | 132720848 | Lung Alveolar Epithelium | 0.05 | 0.91 | intron | FSTL4 | U |
| 2844 | chr15 | 95850339 | 95850565 | Lung Alveolar Epithelium | 0.05 | 0.91 | intron | LOC400456 | U |
| 2845 | chr11 | 352849071 | 35285182 | Lung Alveolar Epithelium | 0.07 | 0.93 | intron | SLC1A2 | U |
| 2846 | chr10 | 80179638 | 80179921 | Lung Alveolar Epithelium | 0.04 | 0.9 | Intergenic | LINC00595 | U |
| 2847 | chr2 | 227714792 | 227715182 | Lung Alveolar Epithelium | 0.05 | 0.91 | intron | RHBDD1 | U |
| 2848 | chr13 | 27754052 | 27754186 | Lung Alveolar Epithelium | 0.03 | 0.88 | Intergenic | USP12 | U |
| 2849 | chr7 | 158595013 | 158595183 | Lung Alveolar Epithelium | 0.03 | 0.88 | intron | ESYT2 | U |
| 2850 | chr10 | 25352752 | 25352978 | Lung Alveolar Epithelium | 0.1 | 0.95 | Intergenic | THNSL1 | U |
| 2851 | chr1 | 94296799 | 94297055 | Lung Alveolar Epithelium | 0.07 | 0.92 | intron | BCAR3 | U |
| 2852 | chr11 | 121367621 | 121367913 | Lung Alveolar Epithelium | 0.03 | 0.88 | intron | SORL1 | U |
| 2853 | chr10 | 81345579 | 81345701 | Lung Alveolar Epithelium | 0.02 | 0.85 | Intergenic | SFTPA1 | U |
| 2854 | chr11 | 46200227 | 46200478 | Lung Alveolar Epithelium | 0.08 | 0.91 | Intergenic | PHF21A | U |
| 2855 | chr2 | 85893108 | 85893606 | Lung Alveolar Epithelium | 0.07 | 0.9 | intron | SFTPB | U |
| 2856 | chr5 | 1546436 | 1546597 | Lung Alveolar Epithelium | 0.05 | 0.87 | Intergenic | LPCAT1 | U |
| 2857 | chr7 | 87920503 | 87920757 | Lung Alveolar Epithelium | 0.07 | 0.89 | intron | STEAP4 | U |
| 2858 | chr11 | 27325873 | 27326147 | Lung Alveolar Epithelium | 0.04 | 0.86 | Intergenic | CCDC34 | U |
| 2859 | chr10 | 74176410 | 74176729 | Lung Alveolar Epithelium | 0.06 | 0.88 | intron | MICU1 | U |
| 2860 | chr17 | 15937103 | 15937495 | Lung Alveolar Epithelium | 0.12 | 0.94 | intron | NCOR1 | U |
| 2861 | chr8 | 12525700 | 12525832 | Lung Alveolar Epithelium | 0.11 | 0.92 | Intergenic | LOC729732 | U |
| 2862 | chr13 | 113438392 | 113438560 | Lung Alveolar Epithelium | 0.07 | 0.88 | intron | ATP11A | U |
| 2863 | chr9 | 138096180 | 138096362 | Lung Alveolar Epithelium | 0.03 | 0.84 | Intergenic | LOC401557 | U |
| 2864 | chr4 | 25515327 | 25515665 | Lung Alveolar Epithelium | 0.07 | 0.88 | Intergenic | ANAPC4 | U |
| 2865 | chr2 | 25851442 | 25851858 | Lung Alveolar Epithelium | 0.11 | 0.92 | intron | DTNB | U |
| 2866 | chr18 | 9856637 | 19356774 | Lung Alveolar Epithelium | 0.11 | 0.91 | intron | MIB1 | U |
| 2867 | chr10 | 33997244 | 33997405 | Lung Alveolar Epithelium | 0.04 | 0.84 | Intergenic | LINC00838 | U |
| 2868 | chr16 | 69133299 | 69133521 | Lung Alveolar Epithelium | 0.1 | 0.9 | Intergenic | HAS3 | U |
| 2869 | chr6 | 38572720 | 38573038 | Lung Alveolar Epithelium | 0.11 | 0.91 | intron | BTBD9 | U |
| 2870 | chr2 | 65602434 | 65602899 | Lung Alveolar Epithelium | 0.13 | 0.93 | intron | SPRED2 | U |
| 2871 | chr2 | 109895272 | 109895384 | Lung Alveolar Epithelium | 0.1 | 0.89 | intron | SH3RF3 | U |
| 2872 | chr10 | 3212788 | 3212986 | Lung Alveolar Epithelium | 0.1 | 0.89 | intron | PITRM1 | U |
| 2873 | chr2 | 35892633 | 85892909 | Lung Alveolar Epithelium | 0.06 | 0.85 | exon | SFTPB | U |
| 2874 | chr10 | 73956581 | 73956730 | Lung Alveolar Epithelium | 0.1 | 0.88 | exon | ASCC1 | U |
| 2875 | chr5 | 132720911 | 132721184 | Lung Alveolar Epithelium | 0.1 | 0.88 | intron | FSTL4 | U |
| 2876 | chr18 | 3512750 | 3513056 | Lung Alveolar Epithelium | 0.09 | 0.87 | intron | DLGAP1 | U |
| 2877 | chr18 | 11585537 | 11585881 | Lung Alveolar Epithelium | 0.08 | 0.86 | Intergenic | GNAL | U |
| 2878 | chr3 | 48338671 | 48339047 | Lung Alveolar Epithelium | 0.1 | 0.9 | intron | NMEG | U |
| 2879 | chr6 | 150714124 | 150714583 | Lung Alveolar Epithelium | 0.1 | 0.9 | intron | IYD | U |
| 2880 | chr16 | 31523180 | 31523661 | Lung Alveolar Epithelium | 0.09 | 0.87 | Intergenic | C16orf58 | U |
| 2881 | chr4 | 148745488 | 148745733 | Lung Alveolar Epithelium | 0.11 | 0.88 | intron | ARHGAP10 | U |
| 2882 | chr11 | 46959181 | 46959324 | Lung Alveolar Epithelium | 0.15 | 0.91 | intron | C11orf49 | U |
| 2883 | chr18 | 22059526 | 22059718 | Lung Alveolar Epithelium | 0.11 | 0.87 | exon, TTS | HRH4, HRH4 | U |
| 2884 | chr1 | 200393978 | 200394246 | Lung Alveolar Epithelium | 0.08 | 0.84 | Intergenic | ZNF281 | U |
| 2885 | chr16 | 47961569 | 47961782 | Lung Alveolar Epithelium | 0.14 | 0.89 | Intergenic | ABCC12 | U |
| 2886 | chr13 | 27657033 | 27657130 | Lung Alveolar Epithelium | 0.23 | 0.97 | intron | USP12 | U |
| 2887 | chr17 | 47496663 | 47496701 | Lung Alveolar Epithelium | 0.18 | 0.91 | Intergenic | PHB | U |
| 2888 | chr3 | 171292399 | 171292700 | Lung Alveolar Epithelium | 0.14 | 0.87 | Intergenic | TNIK | U |
| 2889 | chr4 | 6180089 | 6180440 | Lung Alveolar Epithelium | 0.11 | 0.84 | intron | JAKMIP1 | U |
| 2890 | chr1 | 1983574 | 1983759 | Lung Alveolar Epithelium | 0.14 | 0.86 | intron | PRKCZ | U |
| 2891 | chr12 | 122486051 | 122486455 | Lung Alveolar Epithelium | 0.21 | 0.93 | intron | BCL7A | U |
| 2892 | chr7 | 65587218 | 65587319 | Lung Alveolar Epithelium | 0.22 | 0.93 | intron | CRCP | U |
| 2893 | chr9 | 130694348 | 130694514 | Lung Alveolar Epithelium | 0.18 | 0.89 | Intergenic | PIP5KL1 | U |
| 2894 | chr9 | 127823530 | 127823864 | Lung Alveolar Epithelium | 0.24 | 0.95 | intron | SCAI | U |
| 2895 | chr1 | 1435888 | 1436130 | Lung Alveolar Epithelium | 0.15 | 0.85 | Intergenic | ATAD3A | U |
| 2896 | chr7 | 97767447 | 97767608 | Lung Alveolar Epithelium | 0.24 | 0.93 | intron | LMTK2 | U |
| 2897 | chr12 | 104911000 | 104911372 | Lung Alveolar Epithelium | 0.26 | 0.94 | intron | CHST11 | U |
| 2898 | chr17 | 80071399 | 80071607 | Lung Alveolar Epithelium | 0.24 | 0.9 | intron | CCDC57 | U |
| 2899 | chr1 | 167740172 | 167740485 | Lung Alveolar Epithelium | 0.27 | 0.92 | intron | MPZL1 | U |
| 2900 | chr6 | 151583157 | 151583385 | Lung Alveolar Epithelium | 0.08 | 0.93 | intron | AKAP12 | U |
| 2901 | chr18 | 61135163 | 61135409 | Lung Alveolar Epithelium | 0.1 | 0.9 | Intergenic | SERPINB5 | U |
| 2902 | chr10 | 93396090 | 93396444 | Lung Alveolar Epithelium | 0.09 | 0.87 | Intergenic | PPP1R3C | U |
| 2903 | chr2 | 232035234 | 232035440 | Lung Alveolar Epithelium | 0.23 | 0.92 | exon | PSMD1 | U |
| 2904 | chr6 | 138883706 | 138884001 | Lung Alveolar Epithelium | 0.05 | 0.94 | intron | NHSL1 | U |
| 2905 | chr16 | 678028 | 678173 | Lung Alveolar Epithelium | 0.03 | 0.91 | exon | RAB40C | U |
| 2906 | chr2 | 97305286 | 97305541 | Lung Alveolar Epithelium | 0.06 | 0.94 | Intergenic | KANSL3 | U |
| 2907 | chr22 | 24928939 | 24929341 | Lung Alveolar Epithelium | 0.06 | 0.92 | Intergenic | GUCD1 | U |
| 2908 | chr1 | 244823723 | 244823817 | Lung Alveolar Epithelium | 0.1 | 0.96 | intron | DESI2 | U |
| 2909 | chr21 | 33175293 | 33175675 | Lung Alveolar Epithelium | 0.03 | 0.89 | Intergenic | HUNK | U |
| 2910 | chr7 | 2473795 | 2474257 | Lung Alveolar Epithelium | 0.05 | 0.9 | exon | CHST12 | U |
| 2911 | chr2 | 185490785 | 185490895 | Lung Alveolar Epithelium | 0.07 | 0.91 | intron | ZNF804A | U |
| 2912 | chr14 | 62002256 | 62002702 | Lung Alveolar Epithelium | 0.08 | 0.92 | intron | PRKCH | U |
| 2913 | chr2 | 160086171 | 160086571 | Lung Alveolar Epithelium | 0.08 | 0.91 | exon | TANC1 | U |
| 2914 | chr17 | 57185537 | 57185818 | Lung Alveolar Epithelium | 0.15 | 0.95 | Intergenic | TRIM37 | U |
| 2915 | chr11 | 67781212 | 67781525 | Lung Alveolar Epithelium | 0.13 | 0.91 | intron | ALDH3B1 | U |
| 2916 | chr2 | 61452835 | 61453241 | Lung Alveolar Epithelium | 0.19 | 0.94 | intron | USP34 | U |
| 2917 | chr3 | 179449170 | 179449589 | Lung Alveolar Epithelium | 0.02 | 0.91 | intron | USP13 | U |
| 2918 | chr18 | 72670023 | 72670206 | Lung Alveolar Epithelium | 0.02 | 0.89 | intron | ZNF407 | U |
| 2919 | chr20 | 49179013 | 49179362 | Lung Alveolar Epithelium | 0.06 | 0.91 | intron | PTPN1 | U |
| 2920 | chr15 | 42129252 | 42129616 | Lung Alveolar Epithelium | 0.06 | 0.91 | intron, exon | JMJD7, JMJD7 | U |
| 2921 | chr2 | 98870904 | 98871360 | Lung Alveolar Epithelium | 0.05 | 0.9 | intron | VWA3B | U |
| 2922 | chr2 | 73723864 | 73724126 | Lung Alveolar Epithelium | 0.09 | 0.93 | intron | ALMS1 | U |
| 2923 | chr1 | 252003741 | 25200693 | Lung Alveolar Epithelium | 0.06 | 0.9 | Intergenic | RUNX3 | U |
| 2924 | chr6 | 127669485 | 127669890 | Lung Alveolar Epithelium | 0.1 | 0.94 | Intergenic | ECHDC1 | U |
| 2925 | chr14 | 63872855 | 63872912 | Lung Alveolar Epithelium | 0.1 | 0.93 | intron | PPP2R5E | U |
| 2926 | chr22 | 30907485 | 30907625 | Lung Alveolar Epithelium | 0.05 | 0.88 | Intergenic | SEC14L4 | U |
| 2927 | chr22 | 30216739 | 30216883 | Lung Alveolar Epithelium | 0.1 | 0.92 | intron | ASCC2 | U |
| 2928 | chr12 | 107272882 | 107273121 | Lung Alveolar Epithelium | 0.06 | 0.88 | intron | RIC8B | U |
| 2929 | chr18 | 12849921 | 12850413 | Lung Alveolar Epithelium | 0.06 | 0.88 | intron | PTPN2 | U |
| 2930 | chr3 | 37765199 | 37765284 | Lung Alveolar Epithelium | 0.14 | 0.93 | intron | ITGA9 | U |
| 2931 | chr10 | 135030800 | 135030993 | Lung Alveolar Epithelium | 0.05 | 0.84 | intron | KNDC1 | U |
| 2932 | chr12 | 131478682 | 131478883 | Lung Alveolar Epithelium | 0.13 | 0.92 | intron | GPR133 | U |
| 2933 | chr2 | 100812090 | 100812519 | Lung Alveolar Epithelium | 0.09 | 0.88 | Intergenic | AFF3 | U |
| 2934 | chr9 | 2272215 | 2272359 | Lung Alveolar Epithelium | 0.15 | 0.93 | Intergenic | SMARCA2 | U |
| 2935 | chr14 | 59165532 | 59165760 | Lung Alveolar Epithelium | 0.09 | 0.87 | Intergenic | DACT1 | U |
| 2936 | chr16 | 11398056 | 11398454 | Lung Alveolar Epithelium | 0.14 | 0.92 | Intergenic | PRM1 | U |
| 2937 | chr17 | 78587637 | 78988060 | Lung Alveolar Epithelium | 0.14 | 0.92 | intron | RPTOR | U |
| 2938 | chr14 | 103363706 | 103363938 | Lung Alveolar Epithelium | 0.13 | 0.9 | intron | TRAF3 | U |
| 2939 | chr1 | 231037988 | 231038262 | Lung Alveolar Epithelium | 0.17 | 0.94 | Intergenic | C1orf198 | U |
| 2940 | chr9 | 90154688 | 90154983 | Lung Alveolar Epithelium | 0.18 | 0.95 | intron | DAPK1 | U |
| 2941 | chr1 | 87607301 | 87607738 | Lung Alveolar Epithelium | 0.11 | 0.88 | intron | LOC339524 | U |
| 2942 | chr6 | 14941192 | 14941688 | Lung Alveolar Epithelium | 0.16 | 0.93 | Intergenic | JARID2 | U |
| 2943 | chr5 | 158567331 | 158567722 | Lung Alveolar Epithelium | 0.16 | 0.92 | intron | SERAC1 | U |
| 2944 | chr4 | 1637282 | 1637720 | Lung Alveolar Epithelium | 0.15 | 0.91 | Intergenic | FAM53A | U |
| 2945 | chr7 | 98601588 | 98601745 | Lung Alveolar Epithelium | 0.16 | 0.91 | intron | TRRAP | U |
| 2946 | chr9 | 1029071981 | 102907628 | Lung Alveolar Epithelium | 0.17 | 0.91 | intron | INVS | U |
| 2947 | chr15 | 80217246 | 80217531 | Lung Alveolar Epithelium | 0.23 | 0.93 | TTS, promoter-TSS | C15orf37, ST20 | U |
| 2948 | chr9 | 38075126 | 38075485 | Lung Alveolar Epithelium | 0.24 | 0.94 | Intergenic | SHB | U |
| 2949 | chr16 | 3139060 | 3139148 | Lung Alveolar Epithelium | 0.24 | 0.93 | exon | ZSCAN10 | U |
| 2950 | chr22 | 29203527 | 29203817 | Lung Alveolar Epithelium | 0.25 | 0.94 | Intergenic | XBP1 | U |
| 2951 | chr19 | 7158237 | 7158339 | Lung Alveolar Epithelium | 0.26 | 0.92 | intron | INSR | U |
| 2952 | chr22 | 47197735 | 47198162 | Lung Alveolar Epithelium | 0.28 | 0.94 | intron | TBC1D22A | U |
| 2953 | chr1 | 150748460 | 150748752 | Lung Alveolar Epithelium | 0.35 | 0.93 | Intergenic | CTSS | U |
| 2954 | chr5 | 87439252 | 87439630 | Lung Alveolar Epithelium | 0.94 | 0.16 | Intergenic | TMEM1618 | M |
| 2955 | chr4 | 57521209 | 57521311 | Lung Alveolar Epithelium | 0.78 | 0.19 | intron | HOPX | M |
| 2956 | chr13 | 27050627 | 27050868 | Lung Alveolar Epithelium | 0.7 | 0.14 | Intergenic | WASF3 | M |
| 2957 | chr14 | 91710906 | 91711100 | Lung Alveolar Epithelium | 0.63 | 0.16 | promoter-TSS | GPR68 | M |
| 2958 | chr19 | 18706051 | 18706131 | Lung Alveolar Epithelium | 0.63 | 0.26 | intron | CRLF1 | M |
| 2959 | chr19 | 49112138 | 49112279 | Lung Alveolar Epithelium | 0.63 | 0.28 | intron | FAM83E | M |
| 2960 | chr3 | 139082331 | 13908303 | Lung Alveolar Epithelium | 0.61 | 0.27 | intron | WNT7A | M |
| 2961 | chr3 | 44063807 | 44064029 | Lung Alveolar Epithelium | 0.79 | 0.09 | Intergenic | MIR138-1 | M |
| 2962 | chr19 | 17008520 | 17008796 | Lung Alveolar Epithelium | 0.78 | 0.1 | intron | CPAMD8 | M |
| 2963 | chr2 | 111875094 | 111875507 | Lung Alveolar Epithelium | 0.71 | 0.03 | exon | ACOXL | M |
| 2964 | chr19 | 17007039 | 17007486 | Lung Alveolar Epithelium | 0.75 | 0.07 | intron | CPAMD8 | M |
| 2965 | chr6 | 152623159 | 152623510 | Lung Alveolar Epithelium | 0.64 | 0.02 | intron | SYNE1 | M |
| 2966 | chr5 | 87440121 | 87440509 | Lung Alveolar Epithelium | 0.76 | 0.14 | Intergenic | TMEM161B | M |
| 2967 | chr4 | 57521336 | 57521507 | Lung Alveolar Epithelium | 0.65 | 0.04 | intron | HOPX | M |
| 2968 | chr19 | 17008817 | 17008852 | Lung Alveolar Epithelium | 0.73 | 0.15 | intron | CPAMD8 | M |
| 2969 | chr3 | 44063289 | 44063326 | Lung Alveolar Epithelium | 0.72 | 0.15 | Intergenic | MIR138-1 | M |
| 2970 | chr19 | 11492629 | 11492913 | Lung Alveolar Epithelium | 0.71 | 0.15 | exon, intron | EPOR, EPOR | M |
| 2971 | chr10 | 122708511 | 122708747 | Lung Alveolar Epithelium | 0.63 | 0.09 | intron | MIR5694 | M |
| 2972 | chr5 | 3594139 | 3594432 | Lung Alveolar Epithelium | 0.67 | 0.14 | Intergenic | IRX1 | M |
| 2973 | chr19 | 11492268 | 11492404 | Lung Alveolar Epithelium | 0.58 | 0.06 | exon | EPOR | M |
| 2974 | chr5 | 3606587 | 3607009 | Lung Alveolar Epithelium | 0.63 | 0.14 | Intergenic | IRX1 | M |
| 2975 | chr4 | 122301277 | 122301447 | Lung Alveolar Epithelium | 0.61 | 0.13 | intron | QRFPR | M |
| 2976 | chr1 | 47974491 | 47974712 | Lung Alveolar Epithelium | 0.61 | 0.13 | Intergenic | FOXDZ | M |
| 2977 | chr19 | 104072143 | 10407260 | Lung Alveolar Epithelium | 0.63 | 0.16 | exon | ICAM5 | M |
| 2978 | chr8 | 102038446 | 102038850 | Lung Alveolar Epithelium | 0.61 | 0.14 | Intergenic | FLI42969 | M |
| 2979 | chr16 | 23408418 | 23408836 | Lung Bronchial Epithelium | 0.05 | 0.93 | intron | COG7 | U |
| 2980 | chr8 | 26293472 | 26293783 | Lung Bronchial Epithelium | 0.08 | 0.92 | Intergenic | BNIP3L | U |
| 2981 | chr17 | 56027572 | 56027663 | Lung Bronchial Epithelium | 0.1 | 0.94 | intron | CUEDC1 | U |
| 2982 | chr17 | 64274922 | 64275067 | Lung Bronchial Epithelium | 0.1 | 0.94 | Intergenic | PRKCA | U |
| 2983 | chr8 | 19268415 | 19268524 | Lung Bronchial Epithelium | 0.09 | 0.92 | intron | CSGALNACT1 | U |
| 2984 | chr17 | 8209419 | 8209553 | Lung Bronchial Epithelium | 0.07 | 0.9 | Intergenic | ARHGEF15 | U |
| 2985 | chr8 | 59583884 | 59584023 | Lung Bronchial Epithelium | 0.04 | 0.87 | Intergenic | NSMAF | U |
| 2986 | chr5 | 132231880 | 132232256 | Lung Bronchial Epithelium | 0.04 | 0.87 | exon | AFF4 | U |
| 2987 | chr11 | 120077845 | 120078073 | Lung Bronchial Epithelium | 0.1 | 0.92 | Intergenic | OAF | U |
| 2988 | chr17 | 64571956 | 64572372 | Lung Bronchial Epithelium | 0.12 | 0.94 | intron | PRKCA | U |
| 2989 | chr21 | 33872620 | 33873117 | Lung Bronchial Epithelium | 0.11 | 0.92 | intron | EVA1C | U |
| 2990 | chr12 | 98213200 | 98213306 | Lung Bronchial Epithelium | 0.09 | 0.89 | Intergenic | MIR4303 | U |
| 2991 | chr18 | 44084653 | 44084849 | Lung Bronchial Epithelium | 0.06 | 0.84 | intron | LOXHD1 | U |
| 2992 | chr3 | 147749806 | 147750024 | Lung Bronchial Epithelium | 0.05 | 0.82 | Intergenic | ZIC1 | U |
| 2993 | chr17 | 53331547 | 53331840 | Lung Bronchial Epithelium | 0.15 | 0.91 | Intergenic | HLF | U |
| 2994 | chr8 | 59582952 | 59583311 | Lung Bronchial Epithelium | 0.12 | 0.88 | Intergenic | NSMAF | U |
| 2995 | chr8 | 72269986 | 72270116 | Lung Bronchial Epithelium | 0.11 | 0.86 | intron | EYA1 | U |
| 2996 | chr7 | 64711787 | 64711972 | Lung Bronchial Epithelium | 0.08 | 0.83 | Intergenic | SNF92 | U |
| 2997 | chr3 | 57143301 | 57143517 | Lung Bronchial Epithelium | 0.19 | 0.94 | intron | IL17RD | U |
| 2998 | chr2 | 231426805 | 231427158 | Lung Bronchial Epithelium | 0.14 | 0.85 | Intergenic | LOC151475 | U |
| 2999 | chr2 | 62030600 | 62030903 | Lung Bronchial Epithelium | 0.22 | 0.92 | Intergenic | FAM161A | U |
| 3000 | chr16 | 1752224 | 1752508 | Lung Bronchial Epithelium | 0.32 | 0.93 | TTS | HN1L | U |
| 3001 | chr1 | 155691915 | 155692294 | Lung Bronchial Epithelium | 0.38 | 0.95 | intron | DAP3 | U |
| 3002 | chr6 | 107980870 | 107981125 | Lung Bronchial Epithelium | 0.05 | 0.93 | exon | SOBP | U |
| 3003 | chr5 | 137191290 | 137191665 | Lung Bronchial Epithelium | 0.04 | 0.92 | Intergenic | MYOT | U |
| 3004 | chr13 | 80201369 | 80201594 | Lung Bronchial Epithelium | 0.04 | 0.9 | Intergenic | NDFIP2-AS1 | U |
| 3005 | chr17 | 53331611 | 53331841 | Lung Bronchial Epithelium | 0.06 | 0.92 | Intergenic | HLF | U |
| 3006 | chr2 | 33447183 | 33447527 | Lung Bronchial Epithelium | 0.08 | 0.93 | intron | LTBP1 | U |
| 3007 | chr2 | 61359540 | 61359941 | Lung Bronchial Epithelium | 0.05 | 0.9 | intron | KIAA1841 | U |
| 3008 | chr2 | 206421572 | 206421663 | Lung Bronchial Epithelium | 0.03 | 0.87 | intron | PARD38 | U |
| 3009 | chr10 | 13789249 | 13789452 | Lung Bronchial Epithelium | 0.08 | 0.92 | intron | FRMD4A | U |
| 3010 | chr2 | 68057829 | 68058240 | Lung Bronchial Epithelium | 0.05 | 0.89 | Intergenic | C1D | U |
| 3011 | chr21 | 36192712 | 36192750 | Lung Bronchial Epithelium | 0.11 | 0.93 | TTS | RUNX1 | U |
| 3012 | chr4 | 124277967 | 124278127 | Lung Bronchial Epithelium | 0.05 | 0.87 | Intergenic | SPRY1 | U |
| 3013 | chr14 | 90006939 | 90007031 | Lung Bronchial Epithelium | 0.14 | 0.95 | intron | FOXN3 | U |
| 3014 | chr16 | 4345479 | 4345610 | Lung Bronchial Epithelium | 0.09 | 0.9 | Intergenic | TFAP4 | U |
| 3015 | chr16 | 14108508 | 14108704 | Lung Bronchial Epithelium | 0.05 | 0.86 | Intergenic | MKL2 | U |
| 3016 | chr11 | 27461641 | 27461890 | Lung Bronchial Epithelium | 0.11 | 0.92 | intron | LGR4 | U |
| 3017 | chr10 | 70192040 | 70192309 | Lung Bronchial Epithelium | 0.12 | 0.93 | exon | DNA2 | U |
| 3018 | chr17 | 71045999 | 71046291 | Lung Bronchial Epithelium | 0.08 | 0.89 | intron | SLC39A11 | U |
| 3019 | chr2 | 97080460 | 97080595 | Lung Bronchial Epithelium | 0.1 | 0.9 | Intergenic | NCAPH | U |
| 3020 | chr3 | 4401572 | 4401791 | Lung Bronchial Epithelium | 0.12 | 0.92 | TTS, Intergenic | SUMF1, SETMAR | U |
| 3021 | chr16 | 58613625 | 58613880 | Lung Bronchial Epithelium | 0.12 | 0.92 | intron | CNOT1 | U |
| 3022 | chr3 | 141828866 | 141829164 | Lung Bronchial Epithelium | 0.12 | 0.92 | intron | TFDP2 | U |
| 3023 | chr2 | 233979823 | 233979903 | Lung Bronchial Epithelium | 0.06 | 0.85 | intron | INPP5D | U |
| 3024 | chr2 | 28941738 | 28942085 | Lung Bronchial Epithelium | 0.04 | 0.83 | Intergenic | PPP1CB | U |
| 3025 | chr15 | 84557339 | 84557565 | Lung Bronchial Epithelium | 0.11 | 0.89 | intron | ADAMTSL3 | U |
| 3026 | chr8 | 32160407 | 32160473 | Lung Bronchial Epithelium | 0.08 | 0.85 | intron | NRG1 | U |
| 3027 | chr17 | 56027864 | 56027995 | Lung Bronchial Epithelium | 0.08 | 0.85 | intron | CUEDC1 | U |
| 3028 | chr18 | 75987918 | 75988365 | Lung Bronchial Epithelium | 0.09 | 0.86 | Intergenic | SALL3 | U |
| 3029 | chr4 | 956326501 | 95632851 | Lung Bronchial Epithelium | 0.14 | 0.9 | Intergenic | PDLIM5 | U |
| 3030 | chr19 | 47266433 | 47266803 | Lung Bronchial Epithelium | 0.13 | 0.89 | Intergenic | STRN4 | U |
| 3031 | chr11 | 67488307 | 67488744 | Lung Bronchial Epithelium | 0.12 | 0.88 | Intergenic | ALDH382 | U |
| 3032 | chr17 | 4549899 | 4549954 | Lung Bronchial Epithelium | 0.15 | 0.9 | Intergenic | ALOX15 | U |
| 3033 | chr2 | 118875322 | 118875465 | Lung Bronchial Epithelium | 0.12 | 0.87 | Intergenic | INSIG2 | U |
| 3034 | chr12 | 70367811 | 70368240 | Lung Bronchial Epithelium | 0.09 | 0.84 | Intergenic | RAB3IP | U |
| 3035 | chr1 | 168809120 | 168809550 | Lung Bronchial Epithelium | 0.11 | 0.86 | Intergenic | LINC00626 | U |
| 3036 | chr8 | 122873916 | 122874072 | Lung Bronchial Epithelium | 0.15 | 0.89 | Intergenic | HAS2 | U |
| 3037 | chr12 | 52071638 | 52071882 | Lung Bronchial Epithelium | 0.07 | 0.81 | intron | SCN8A | U |
| 3038 | chr13 | 242484861 | 24248751 | Lung Bronchial Epithelium | 0.1 | 0.84 | exon | TNFRSF19 | U |
| 3039 | chr13 | 25509965 | 25510435 | Lung Bronchial Epithelium | 0.1 | 0.84 | intron | TPTE2P1 | U |
| 3040 | chr10 | 74846359 | 74846552 | Lung Bronchial Epithelium | 0.16 | 0.89 | intron | P4HA1 | U |
| 3041 | chr6 | 158196250 | 158196563 | Lung Bronchial Epithelium | 0.11 | 0.84 | Intergenic | SNX9 | U |
| 3042 | chr9 | 123360632 | 123360948 | Lung Bronchial Epithelium | 0.21 | 0.94 | Intergenic | CDK5RAP2 | U |
| 3043 | chr10 | 129014611 | 129014940 | Lung Bronchial Epithelium | 0.15 | 0.88 | intron | DOCK1 | U |
| 3044 | chr2 | 218071757 | 218072113 | Lung Bronchial Epithelium | 0.07 | 0.8 | Intergenic | TNP1 | U |
| 3045 | chr1 | 24855202 | 24855636 | Lung Bronchial Epithelium | 0.16 | 0.89 | intron | RCAN3 | U |
| 3046 | chr13 | 50142996 | 50143481 | Lung Bronchial Epithelium | 0.17 | 0.9 | intron | RCBTB1 | U |
| 3047 | chr3 | 13287610 | 13287766 | Lung Bronchial Epithelium | 0.19 | 0.91 | Intergenic | NUP210 | U |
| 3048 | chr19 | 10271487 | 10271791 | Lung Bronchial Epithelium | 0.23 | 0.95 | intron | DNMT1 | U |
| 3049 | chr12 | 39751580 | 39751899 | Lung Bronchial Epithelium | 0.22 | 0.94 | intron | KIF21A | U |
| 3050 | chr7 | 70256599 | 70256930 | Lung Bronchial Epithelium | 0.2 | 0.92 | exor | AUTS2 | U |
| 3051 | chr4 | 124427917 | 124428276 | Lung Bronchial Epithelium | 0.21 | 0.93 | Intergenic | SPRY1 | U |
| 3052 | chr7 | 92105316 | 92105449 | Lung Bronchial Epithelium | 0.17 | 0.88 | Intergenic | GATAD1 | U |
| 3053 | chr8 | 146007799 | 146007961 | Lung Bronchial Epithelium | 0.15 | 0.86 | intron | ZNF34 | U |
| 3054 | chr2 | 74446080 | 74446564 | Lung Bronchial Epithelium | 0.13 | 0.84 | intron | SLC4AS | U |
| 3055 | chr1 | 21693223 | 21693342 | Lung Bronchial Epithelium | 0.22 | 0.92 | Intergenic | NBPF3 | U |
| 3056 | chr17 | 7423900 | 7424121 | Lung Bronchial Epithelium | 0.2 | 0.9 | Intergenic | TNFSF12-TNFSF13 | U |
| 3057 | chr12 | 16917346 | 16917592 | Lung Bronchial Epithelium | 0.12 | 0.82 | Intergenic | LMO3 | U |
| 3058 | chr6 | 1408433283 | 140843734 | Lung Bronchial Epithelium | 0.17 | 0.87 | Intergenic | MIR4465 | U |
| 3059 | chr8 | 123543820 | 123544281 | Lung Bronchial Epithelium | 0.17 | 0.87 | Intergenic | ZHX2 | U |
| 3060 | chr7 | 40647660 | 40647976 | Lung Bronchial Epithelium | 0.15 | 0.84 | intron | C7orf10 | U |
| 3061 | chr12 | 4000795 | 4001005 | Lung Bronchial Epithelium | 0.19 | 0.87 | Intergenic | PARP11 | U |
| 3062 | chr1 | 230085990 | 230086147 | Lung Bronchial Epithelium | 0.21 | 0.88 | Intergenic | GALNT2 | U |
| 3063 | chr6 | 122869021 | 122869240 | Lung Bronchial Epithelium | 0.21 | 0.88 | intron | PKIB | U |
| 3064 | chr16 | 29586452 | 29586631 | Lung Bronchial Epithelium | 0.22 | 0.88 | Intergenic | SLC7A5P1 | U |
| 3065 | chr1 | 113369423 | 113369766 | Lung Bronchial Epithelium | 0.22 | 0.88 | Intergenic | AKR7A2P1 | U |
| 3066 | chr3 | 5759429 | 5759837 | Lung Bronchial Epithelium | 0.19 | 0.85 | Intergenic | MIR4790 | U |
| 3067 | chr17 | 20338997 | 20339458 | Lung Bronchial Epithelium | 0.19 | 0.85 | Intergenic | LGALS9B | U |
| 3068 | chr1 | 10702185 | 10702373 | Lung Bronchial Epithelium | 0.14 | 0.79 | intron | CASZ1 | U |
| 3069 | chr13 | 50143575 | 50143912 | Lung Bronchial Epithelium | 0.28 | 0.93 | intron | RCBTB1 | U |
| 3070 | chr10 | 21621413 | 21621774 | Lung Bronchial Epithelium | 0.2 | 0.85 | Intergenic | NEBL-AS1 | U |
| 3071 | chr9 | 130261931 | 130262136 | Lung Bronchial Epithelium | 0.29 | 0.93 | intron | LRSAM1 | U |
| 3072 | chr3 | 170186154 | 170186327 | Lung Bronchial Epithelium | 0.28 | 0.91 | intron | SLC7A14 | U |
| 3073 | chr21 | 39623442 | 39623667 | Lung Bronchial Epithelium | 0.29 | 0.92 | intron | KCNJ15 | U |
| 3074 | chr2 | 27520111 | 2752237 | Lung Bronchial Epithelium | 0.15 | 0.78 | Intergenic | MYT1L | U |
| 3075 | chr8 | 126844817 | 126845111 | Lung Bronchial Epithelium | 0.27 | 0.89 | Intergenic | LOC100130231 | U |
| 3076 | chr9 | 1654895 | 1655283 | Lung Bronchial Epithelium | 0.22 | 0.84 | Intergenic | SMARCA2 | U |
| 3077 | chr2 | 225670270 | 225670761 | Lung Bronchial Epithelium | 0.27 | 0.89 | intron | DOCK10 | U |
| 3078 | chr15 | 47216695 | 47216791 | Lung Bronchial Epithelium | 0.27 | 0.88 | Intergenic | SEMA6D | U |
| 3079 | chr10 | 99450493 | 99450679 | Lung Bronchial Epithelium | 0.29 | 0.9 | Intergenic | AVPI1 | U |
| 3080 | chr17 | 10043549 | 10043882 | Lung Bronchial Epithelium | 0.26 | 0.87 | intron | GAS7 | U |
| 3081 | chr3 | 125144031 | 125144512 | Lung Bronchial Epithelium | 0.26 | 0.87 | Intergenic | ZNF148 | U |
| 3082 | chr19 | 42907630 | 42908130 | Lung Bronchial Epithelium | 0.32 | 0.93 | intron | LIPE | U |
| 3083 | chr16 | 68249693 | 68250013 | Lung Bronchial Epithelium | 0.35 | 0.95 | intron | NFATC3 | U |
| 3084 | chr12 | 105604559 | 105604915 | Lung Bronchial Epithelium | 0.33 | 0.92 | intron | APPL2 | U |
| 3085 | chr3 | 12413887 | 12414326 | Lung Bronchial Epithelium | 0.3 | 0.89 | intron | PPARG | U |
| 3086 | chr11 | 778593461 | 77859668 | Lung Bronchial Epithelium | 0.33 | 0.91 | Intergenic | ALG8 | U |
| 3087 | chr19 | 41659761 | 41659991 | Lung Bronchial Epithelium | 0.34 | 0.9 | Intergenic | CYP251 | U |
| 3088 | chr13 | 111897872 | 111897971 | Lung Bronchial Epithelium | 0.08 | 0.9 | intron | ARHGEF7 | U |
| 3089 | chr6 | 152651802 | 152652268 | Lung Bronchial Epithelium | 0.08 | 0.84 | exon | SYNE1 | U |
| 3090 | chr10 | 88544748 | 88545188 | Lung Bronchial Epithelium | 0.23 | 0.87 | intron | BMPRIA | U |
| 3091 | chr1 | 164761969 | 164762279 | Lung Bronchial Epithelium | 0.03 | 0.87 | intron | PBX1 | U |
| 3092 | chr9 | 107601248 | 107601574 | Lung Bronchial Epithelium | 0.11 | 0.9 | intron | ABCA1 | U |
| 3093 | chr1 | 1647617841 | 164761954 | Lung Bronchial Epithelium | 0.04 | 0.92 | exon | PBX1 | U |
| 3094 | chr13 | 255610623 | 25561152 | Lung Bronchial Epithelium | 0.07 | 0.88 | Intergenic | TPTE2P1 | U |
| 3095 | chr20 | 20752152 | 20752339 | Lung Bronchial Epithelium | 0.09 | 0.9 | Intergenic | RALGAPA2 | U |
| 3096 | chr20 | 20542933 | 20543154 | Lung Bronchial Epithelium | 0.06 | 0.87 | intron | RALGAPA2 | U |
| 3097 | chr16 | 16022532 | 16022815 | Lung Bronchial Epithelium | 0.13 | 0.93 | Intergenic | ABCC1 | U |
| 3098 | chr14 | 61150822 | 61150872 | Lung Bronchial Epithelium | 0.15 | 0.94 | Intergenic | SIX1 | U |
| 3099 | chr1 | 226003504 | 226003849 | Lung Bronchial Epithelium | 0.1 | 0.88 | intron | EPHX1 | U |
| 3100 | chr22 | 36683736 | 36683948 | Lung Bronchial Epithelium | 0.15 | 0.92 | intron | MYH9 | U |
| 3101 | chr1 | 22092159 | 22092330 | Lung Bronchial Epithelium | 0.14 | 0.85 | intron | USP48 | U |
| 3102 | chr16 | 89189928 | 89189970 | Lung Bronchial Epithelium | 0.23 | 0.89 | intron | ACSF3 | U |
| 3103 | chr12 | 110384902 | 110385148 | Lung Bronchial Epithelium | 0.27 | 0.93 | TTS | GIT2 | U |
| 3104 | chr20 | 36697235 | 36697651 | Lung Bronchial Epithelium | 0.34 | 0.93 | intron | RPRD1B | U |
| 3105 | chr8 | 72469784 | 72470079 | Lung Bronchial Epithelium | 0.96 | 0.15 | Intergenic | EYA1 | M |
| 3106 | chr7 | 19147936 | 19148283 | Lung Bronchial Epithelium | 0.75 | 0.12 | Intergenic | TWIST1 | M |
| 3107 | chr17 | 36101312 | 36101520 | Lung Bronchial Epithelium | 0.67 | 0.08 | intron | HNF18 | M |
| 3108 | chr17 | 36101617 | 36101694 | Lung Bronchial Epithelium | 0.81 | 0.28 | intron | HNF1B | M |
| 3109 | chr5 | 134365897 | 134366390 | Lung Bronchial Epithelium | 0.69 | 0.16 | intron | PITX1 | M |
| 3110 | chr8 | 72470207 | 72470504 | Lung Bronchial Epithelium | 0.95 | 0.09 | Intergenic | EYA1 | M |
| 3111 | chr8 | 72470524 | 72470833 | Lung Bronchial Epithelium | 0.93 | 0.11 | Intergenic | EYA1 | M |
| 3112 | chr14 | 36993562 | 36993880 | Lung Bronchial Epithelium | 0.9 | 0.08 | Intergenic | NKX2-1 | M |
| 3113 | chr8 | 72469494 | 72469643 | Lung Bronchial Epithelium | 0.97 | 0.16 | Intergenic | EYA1 | M |
| 3114 | chr8 | 72471017 | 72471160 | Lung Bronchial Epithelium | 0.9 | 0.1 | Intergenic | EYA1 | M |
| 3115 | chr8 | 72469259 | 72469478 | Lung Bronchial Epithelium | 0.89 | 0.09 | Intergenic | EYA1 | M |
| 3116 | chr14 | 36993918 | 36993963 | Lung Bronchial Epithelium | 0.94 | 0.18 | Intergenic | NKX2-1 | M |
| 3117 | chr14 | 36993345 | 36993518 | Lung Bronchial Epithelium | 0.95 | 0.2 | Intergenic | NKX2-1 | M |
| 3118 | chr14 | 36992248 | 36992379 | Lung Bronchial Epithelium | 0.82 | 0.08 | Intergenic | NKX2-1 | M |
| 3119 | chr14 | 36992388 | 36992488 | Lung Bronchial Epithelium | 0.85 | 0.12 | Intergenic | NKX2-1 | M |
| 3120 | chr14 | 36992090 | 36992210 | Lung Bronchial Epithelium | 0.84 | 0.12 | Intergenic | VKX2-1 | M |
| 3121 | chr14 | 36992526 | 36992748 | Lung Bronchial Epithelium | 0.84 | 0.17 | Intergenic | NKX2-1 | M |
| 3122 | chr5 | 134367238 | 134367397 | Lung Bronchial Epithelium | 0.83 | 0.18 | intron | PITX1 | M |
| 3123 | chr1 | 50891470 | 50891751 | Lung Bronchial Epithelium | 0.73 | 0.1 | Intergenic | DMRTA2 | M |
| 3124 | chr1 | 189724201 | 18972500 | Lung Bronchial Epithelium | 0.74 | 0.12 | intron | PAX7 | M |
| 3125 | chr6 | 16052741 | 1605353 | Lung Bronchial Epithelium | 0.74 | 0.13 | Intergenic | FOXC1 | M |
| 3126 | chr14 | 369940163 | 36994335 | Lung Bronchial Epithelium | 0.69 | 0.09 | Intergenic | NKX2-1 | M |
| 3127 | chr14 | 36976428 | 36976874 | Lung Bronchial Epithelium | 0.67 | 0.09 | intron | SFTA3 | M |
| 3128 | chr9 | 967980 | 968249 | Lung Bronchial Epithelium | 0.62 | 0.1 | exon | DMRT1 | M |
| 3129 | chr14 | 36991185 | 36991324 | Lung Bronchial Epithelium | 0.6 | 0.12 | Intergenic | NKX2-1 | M |
| 3130 | chr17 | 45289451 | 45289569 | Heart Cardiomyocytes | 0.02 | 0.96 | intron | MYL4 | U |
| 3131 | chr11 | 58317179 | 58317528 | Heart Cardiomyocytes | 0.04 | 0.95 | intron | LPXN | U |
| 3132 | chr20 | 50123513 | 50123595 | Heart Cardiomyocytes | 0.03 | 0.93 | intron | NFATC2 | U |
| 3133 | chr1 | 150995966 | 150996022 | Heart Cardiomyocytes | 0.04 | 0.94 | intron | PRUNE | U |
| 3134 | chr10 | 855627 | 855871 | Heart Cardiomyocytes | 0.05 | 0.95 | exon | LARP4B | U |
| 3135 | chr19 | 16495864 | 16496030 | Heart Cardiomyocytes | 0.04 | 0.93 | intron | EPS15L1 | U |
| 3136 | chr9 | 134111086 | 134111578 | Heart Cardiomyocytes | 0.04 | 0.93 | Intergenic | FAM78A | U |
| 3137 | chr19 | 2797235 | 2797659 | Heart Cardiomyocytes | 0.06 | 0.95 | intron | THOP1 | U |
| 3138 | chr14 | 93410391 | 93410462 | Heart Cardiomyocytes | 0.06 | 0.94 | intron | ITPK1 | U |
| 3139 | chr12 | 1752352 | 1752720 | Heart Cardiomyocytes | 0.06 | 0.94 | intron | WNT5B | U |
| 3140 | chr12 | 113712523 | 113712921 | Heart Cardiomyocytes | 0.06 | 0.93 | intron | TPCN1 | U |
| 3141 | chr5 | 31957413 | 31957569 | Heart Cardiomyocytes | 0.05 | 0.92 | intron | PDZD2 | U |
| 3142 | chr1 | 861573173 | 86157429 | Heart Cardiomyocytes | 0.07 | 0.93 | intron | ZNHIT6 | U |
| 3143 | chr15 | 91462055 | 91462511 | Heart Cardiomyocytes | 0.05 | 0.91 | exon | MAN2A2 | U |
| 3144 | chr2 | 39480266 | 39480431 | Heart Cardiomyocytes | 0.1 | 0.95 | intron | MAP4K3 | U |
| 3145 | chr1 | 11906187 | 11906623 | Heart Cardiomyocytes | 0.08 | 0.92 | intron | NPPA | U |
| 3146 | chr12 | 898153481 | 89815641 | Heart Cardiomyocytes | 0.1 | 0.92 | exon | POC1B | U |
| 3147 | chr10 | 71091101 | 71091451 | Heart Cardiomyocytes | 0.13 | 0.94 | intron | HK1 | U |
| 3148 | chr5 | 11175520 | 11175620 | Heart Cardiomyocytes | 0.02 | 0.93 | intron | CTNND2 | U |
| 3149 | chr11 | 11324205 | 11324346 | Heart Cardiomyocytes | 0.04 | 0.95 | intron | GALNT18 | U |
| 3150 | chr8 | 28853345 | 28853580 | Heart Cardiomyocytes | 0.04 | 0.95 | intron | HMBOX1 | U |
| 3151 | chr2 | 10985095 | 10985195 | Heart Cardiomyocytes | 0.02 | 0.92 | Intergenic | PDIA6 | U |
| 3152 | chr6 | 118876312 | 118876516 | Heart Cardiomyocytes | 0.04 | 0.93 | intron | PLN | U |
| 3153 | chr3 | 151599078 | 151599173 | Heart Cardiomyocytes | 0.04 | 0.92 | exon | SUCNR1 | U |
| 3154 | chr7 | 151419637 | 151419798 | Heart Cardiomyocytes | 0.03 | 0.91 | intron | PRKAG2 | U |
| 3155 | chr13 | 31797516 | 31797715 | Heart Cardiomyocytes | 0.06 | 0.94 | intron | B3GALTL | U |
| 3156 | chr21 | 20435 | 29935766 | Heart Cardiomyocytes | 0.04 | 0.92 | Intergenic | INC00161 | U |
| 3157 | chr18 | 19444129 | 19444184 | Heart Cardiomyocytes | 0.09 | 0.96 | intron | MIB1 | U |
| 3158 | chr13 | 113384765 | 113384930 | Heart Cardiomyocytes | 0.05 | 0.92 | intron | ATP11A | U |
| 3159 | chr18 | 21114779 | 21114982 | Heart Cardiomyocytes | 0.06 | 0.93 | intron | NPC1 | U |
| 3160 | chr1 | 74714016 | 74714177 | Heart Cardiomyocytes | 0.04 | 0.9 | intron | TNNI3K | U |
| 3161 | chr1 | 39702578 | 39702776 | Heart Cardiomyocytes | 0.05 | 0.91 | intron | MACF1 | U |
| 3162 | chr8 | 1038790363 | 103879274 | Heart Cardiomyocytes | 0.09 | 0.95 | Intergenic | AZIN1 | U |
| 3163 | chr18 | 20780515 | 20780760 | Heart Cardiomyocytes | 0.07 | 0.93 | intron | CABLES1 | U |
| 3164 | chr21 | 36822397 | 36822539 | Heart Cardiomyocytes | 0.06 | 0.91 | intron | LOC100506403 | U |
| 3165 | chr4 | 186583258 | 186583408 | Heart Cardiomyocytes | 0.06 | 0.91 | intron | SORBS2 | U |
| 3166 | chr16 | 58137284 | 58137468 | Heart Cardiomyocytes | 0.06 | 0.91 | Intergenic | C16orf80 | U |
| 3167 | chr12 | 659008 | 659201 | Heart Cardiomyocytes | 0.07 | 0.92 | intron | B4GALNT3 | U |
| 3168 | chr11 | 11832410 | 11832720 | Heart Cardiomyocytes | 0.07 | 0.92 | Intergenic | USP47 | U |
| 3169 | chr21 | 40723800 | 40724125 | Heart Cardiomyocytes | 0.08 | 0.93 | Intergenic | HMGN1 | U |
| 3170 | chr17 | 79664393 | 79664785 | Heart Cardiomyocytes | 0.04 | 0.89 | intron | HG5 | U |
| 3171 | chr4 | 3168017 | 3168191 | Heart Cardiomyocytes | 0.07 | 0.91 | intron | HTT | U |
| 3172 | chr16 | 31905880 | 31906152 | Heart Cardiomyocytes | 0.12 | 0.96 | intron | ZNF267 | U |
| 3173 | chr2 | 220494796 | 220495131 | Heart Cardiomyocytes | 0.06 | 0.9 | exon | SLC4A3 | U |
| 3174 | chr1 | 197611724 | 197612100 | Heart Cardiomyocytes | 0.07 | 0.91 | exor | DENND1B | U |
| 3175 | chr11 | 724038121 | 72404276 | Heart Cardiomyocytes | 0.06 | 0.9 | intron | ARAP1 | U |
| 3176 | chr6 | 40900691 | 40901188 | Heart Cardiomyocytes | 0.1 | 0.94 | Intergenic | UNC5CL | U |
| 3177 | chr6 | 42840212 | 42840268 | Heart Cardiomyocytes | 0.06 | 0.89 | Intergenic | RPL7L1 | U |
| 3178 | chr19 | 4891305 | 4891425 | Heart Cardiomyocytes | 0.06 | 0.89 | exon | ARRDC5 | U |
| 3179 | chr17 | 1406919 | 1407097 | Heart Cardiomyocytes | 0.08 | 0.91 | intron | INPP5K | U |
| 3180 | chr2 | 220495885 | 220496083 | Heart Cardiomyocytes | 0.08 | 0.91 | intron | SLC4A3 | U |
| 3181 | chr16 | 12221232 | 12221440 | Heart Cardiomyocytes | 0.11 | 0.94 | intron | SNX29 | U |
| 3182 | chr12 | 133317312 | 133317534 | Heart Cardiomyocytes | 0.03 | 0.86 | intron | ANKLE2 | U |
| 3183 | chr12 | 110725576 | 110725808 | Heart Cardiomyocytes | 0.08 | 0.91 | intron | ATP2A2 | U |
| 3184 | chr10 | 855884 | 856183 | Heart Cardiomyocytes | 0.06 | 0.89 | exon | LARP4B | U |
| 3185 | chr20 | 62894192 | 62894503 | Heart Cardiomyocytes | 0.11 | 0.94 | intron | PCMTD2 | U |
| 3186 | chr2 | 220500101 | 220500162 | Heart Cardiomyocytes | 0.12 | 0.94 | exon | SLC4A3 | U |
| 3187 | chr1 | 1560960 | 1561055 | Heart Cardiomyocytes | 0.13 | 0.95 | exon | MIB2 | U |
| 3188 | chr17 | 78862340 | 78862556 | Heart Cardiomyocytes | 0.1 | 0.92 | intron | RPTOR | U |
| 3189 | chr19 | 3965028 | 3965381 | Heart Cardiomyocytes | 0.09 | 0.91 | intron | DAPK3 | U |
| 3190 | chr22 | 47368954 | 47369344 | Heart Cardiomyocytes | 0.12 | 0.94 | intron | TBC1D22A | U |
| 3191 | chr1 | 12060489 | 12060716 | Heart Cardiomyocytes | 0.09 | 0.9 | intron | MFN2 | U |
| 3192 | chr17 | 45290345 | 45290640 | Heart Cardiomyocytes | 0.04 | 0.85 | intron | MYL4 | U |
| 3193 | chr15 | 29067052 | 29067437 | Heart Cardiomyocytes | 0.1 | 0.91 | intron | LOC646278 | U |
| 3194 | chr2 | 106053411 | 106053890 | Heart Cardiomyocytes | 0.08 | 0.89 | intron | FHL2 | U |
| 3195 | chr15 | 44166456 | 44166646 | Heart Cardiomyocytes | 0.14 | 0.94 | exon, TTS | FRMD5, PIN4P1 | U |
| 3196 | chr11 | 47371760 | 47371967 | Heart Cardiomyocytes | 0.14 | 0.94 | intron | MYBPC3 | U |
| 3197 | chr16 | 46780409 | 46780657 | Heart Cardiomyocytes | 0.05 | 0.85 | intron | MYLK3 | U |
| 3198 | chr11 | 47371313 | 47371620 | Heart Cardiomyocytes | 0.11 | 0.91 | exon | MYBPC3 | U |
| 3199 | chr16 | 79577110 | 79577439 | Heart Cardiomyocytes | 0.13 | 0.93 | Intergenic | MAF | U |
| 3200 | chr5 | 134686461 | 134686575 | Heart Cardiomyocytes | 0.15 | 0.94 | intron | H2AFY | U |
| 3201 | chr7 | 1880766 | 1880997 | Heart Cardiomyocytes | 0.15 | 0.94 | intron | MAD1L1 | U |
| 3202 | chr6 | 158320269 | 158320580 | Heart Cardiomyocytes | 0.12 | 0.91 | intron | SNX9 | U |
| 3203 | chr3 | 1232125681 | 123213047 | Heart Cardiomyocytes | 0.12 | 0.91 | TTS | PTPLB | U |
| 3204 | chr2 | 175827352 | 175827495 | Heart Cardiomyocytes | 0.1 | 0.88 | intron | CHN1 | U |
| 3205 | chr4 | 8374310 | 8374401 | Heart Cardiomyocytes | 0.11 | 0.88 | intron | ACOX3 | U |
| 3206 | chr3 | 58271022 | 58271200 | Heart Cardiomyocytes | 0.14 | 0.91 | intron | ABHD6 | U |
| 3207 | chr7 | 101662149 | 101662341 | Heart Cardiomyocytes | 0.16 | 0.93 | intron | CUX1 | U |
| 3208 | chr1 | 1164854 | 1165302 | Heart Cardiomyocytes | 0.14 | 0.91 | intron | SDF4 | U |
| 3209 | chr4 | 16704723 | 1670607 | Heart Cardiomyocytes | 0.15 | 0.9 | exon | FAM53A | U |
| 3210 | chr20 | 2126781 | 2127210 | Heart Cardiomyocytes | 0.15 | 0.9 | exon | STK35 | U |
| 3211 | chr12 | 65076064 | 65076220 | Heart Cardiomyocytes | 0.2 | 0.94 | intron | RASSF3 | U |
| 3212 | chr2 | 134867260 | 134867447 | Heart Cardiomyocytes | 0.22 | 0.96 | Intergenic | MIR3679 | U |
| 3213 | chr9 | 135195492 | 135195734 | Heart Cardiomyocytes | 0.22 | 0.96 | intron | SETX | U |
| 3214 | chr3 | 176989876 | 176990343 | Heart Cardiomyocytes | 0.2 | 0.94 | Intergenic | TBL1XR1 | U |
| 3215 | chr20 | 2123464 | 2123709 | Heart Cardiomyocytes | 0.2 | 0.93 | intron | STK35 | U |
| 3216 | chr12 | 133323784 | 133324163 | Heart Cardiomyocytes | 0.19 | 0.92 | intron | ANKLE2 | U |
| 3217 | chr11 | 2756110 | 2756326 | Heart Cardiomyocytes | 0.22 | 0.94 | intron | KCNQ1 | U |
| 3218 | chr17 | 78937940 | 78938224 | Heart Cardiomyocytes | 0.21 | 0.93 | intron, exon | RPTOR, RPTOR | U |
| 3219 | chr10 | 103727302 | 103727510 | Heart Cardiomyocytes | 0.22 | 0.93 | intron | C10orf76 | U |
| 3220 | chr2 | 100037591 | 100037819 | Heart Cardiomyocytes | 0.23 | 0.94 | intron | REV1 | U |
| 3221 | chr20 | 559823433 | 55982767 | Heart Cardiomyocytes | 0.25 | 0.94 | intron | RBM38 | U |
| 3222 | chr16 | 88019671 | 88020080 | Heart Cardiomyocytes | 0.22 | 0.9 | intron | BANP | U |
| 3223 | chr16 | 87432441 | 87432679 | Heart Cardiomyocytes | 0.29 | 0.93 | exon, intron | MAP1LC3B, MAP1LC3 [symbol] | U |
| 3224 | chr14 | 316422483 | 31642460 | Heart Cardiomyocytes | 0.06 | 0.97 | intron | HECTD1 | U |
| 3225 | chr14 | 238749903 | 23875361 | Heart Cardiomyocytes | 0.03 | 0.92 | intron | MYH6 | U |
| 3226 | chr1 | 230978623 | 230978905 | Heart Cardiomyocytes | 0.06 | 0.93 | exon, intron | C1orf198 | U |
| 3227 | chr11 | 68550868 | 68551336 | Heart Cardiomyocytes | 0.07 | 0.93 | intron | CPT1A | U |
| 3228 | chr22 | 198582221 | 19858452 | Heart Cardiomyocytes | 0.09 | 0.92 | Intergenic | GNB1L | U |
| 3229 | chr22 | 42042134 | 42042506 | Heart Cardiomyocytes | 0.11 | 0.93 | intron | XRCC6 | U |
| 3230 | chr11 | 68550315 | 68550775 | Heart Cardiomyocytes | 0.13 | 0.95 | intron | CPT1A | U |
| 3231 | chr13 | 1133836121 | 113383835 | Heart Cardiomyocytes | 0.03 | 0.92 | intron | ATP11A | U |
| 3232 | chr6 | 504028 | 504466 | Heart Cardiomyocytes | 0.07 | 0.95 | intron | EXOC2 | U |
| 3233 | chr22 | 283744971 | 28374626 | Heart Cardiomyocytes | 0.07 | 0.93 | exon | TTC28 | U |
| 3234 | chr14 | 52477280 | 52477727 | Heart Cardiomyocytes | 0.07 | 0.93 | intron | NID2 | U |
| 3235 | chr17 | 1792185 | 1792459 | Heart Cardiomyocytes | 0.07 | 0.92 | intron | RPA1 | U |
| 3236 | chr16 | 467817303 | 46782205 | Heart Cardiomyocytes | 0.05 | 0.9 | exon, intron | MYLK3, MYLK3 | U |
| 3237 | chr11 | 16877102 | 16877459 | Heart Cardiomyocytes | 0.13 | 0.94 | intron | PLEKHA7 | U |
| 3238 | chr22 | 22053867 | 22053901 | Heart Cardiomyocytes | 0.08 | 0.91 | exon | YPEL1 | U |
| 3239 | chr2 | 220508803 | 220509002 | Heart Cardiomyocytes | 0.07 | 0.89 | Intergenic | SLC4A3 | U |
| 3240 | chr13 | 1133833241 | 113383529 | Heart Cardiomyocytes | 0.1 | 0.92 | intron | ATP11A | U |
| 3241 | chr14 | 742978011 | 74298075 | Heart Cardiomyocytes | 0.12 | 0.94 | Intergenic | PTGR2 | U |
| 3242 | chr10 | 13735916 | 13736208 | Heart Cardiomyocytes | 0.1 | 0.92 | exon | ERMD4A | U |
| 3243 | chr2a | 47570179 | 47570512 | Heart Cardiomyocytes | 0.08 | 0.9 | intron | ARFGEF2 | U |
| 3244 | chr1 | 164810236 | 164810341 | Heart Cardiomyocytes | 0.13 | 0.94 | intron | PBX1 | U |
| 3245 | chr16 | 46781221 | 46781714 | Heart Cardiomyocytes | 0.05 | 0.86 | intron | MYLK3 | U |
| 3246 | chr1 | 1499885 | 1500143 | Heart Cardiomyocytes | 0.11 | 0.91 | intron | SSU72 | U |
| 3247 | chr21 | 432715121 | 43271686 | Heart Cardiomyocytes | 0.17 | 0.95 | intron | PRDM15 | U |
| 3248 | chr14 | 238660213 | 23866412 | Heart Cardiomyocytes | 0.13 | 0.91 | exon | MYHE | U |
| 3249 | chr22 | 42056809 | 42057295 | Heart Cardiomyocytes | 0.16 | 0.94 | intron | XRCC6 | U |
| 3250 | chr10 | 11505339 | 11505563 | Heart Cardiomyocytes | 0.21 | 0.95 | exon | USP6NL | U |
| 3251 | chr6 | 158094254 | 158094625 | Heart Cardiomyocytes | 0.25 | 0.93 | exon | ZDHHC14 | U |
| 3252 | chr11 | 47367776 | 47367872 | Heart Cardiomyocytes | 0.25 | 0.92 | intron | MYBPC3 | U |
| 3253 | chr8 | 141570717 | 141570984 | Heart Cardiomyocytes | 0.28 | 0.92 | intron | AGO2 | U |
| 3254 | chr9 | 134136258 | 134136566 | Heart Cardiomyocytes | 0.3 | 0.93 | exon | FAM7BA | U |
| 3255 | chr2 | 132249139 | 132249263 | Heart Cardiomyocytes | 0.86 | 0.14 | promoter-TSS | MIR4784 | M |
| 3256 | chr3 | 138892681 | 138892934 | Heart Cardiomyocytes | 0.81 | 0.09 | Intergenic | PISRT1 | M |
| 3257 | chr9 | 139636512 | 139636807 | Heart Cardiomyocytes | 0.85 | 0.15 | intron | LCN10 | M |
| 3258 | chr13 | 74862518 | 74862769 | Heart Cardiomyocytes | 0.78 | 0.09 | Intergenic | LINC00381 | M |
| 3259 | chr10 | 98869374 | 98869555 | Heart Cardiomyocytes | 0.88 | 0.2 | intron | SLIT1 | M |
| 3260 | chr13 | 1083738773 | 108374053 | Heart Cardiomyocytes | 0.81 | 0.16 | intron | FAM155A | M |
| 3261 | chr9 | 118915832 | 118916305 | Heart Cardiomyocytes | 0.71 | 0.08 | promoter-TSS, intron | PAPPA, PAPPA | M |
| 3262 | chr16 | 79320968 | 79321297 | Heart Cardiomyocytes | 0.65 | 0.06 | Intergenic | MAF | M |
| 3263 | chr14 | 94405462 | 94405535 | Heart Cardiomyocytes | 0.74 | 0.18 | exon | ASB2 | M |
| 3264 | chr5 | 99606013 | 99606312 | Heart Cardiomyocytes | 0.62 | 0.06 | Intergenic | LOC100133050 | M |
| 3265 | chr15 | 68870038 | 68870242 | Heart Cardiomyocytes | 0.54 | 0.07 | Intergenic | CORO28 | M |
| 3266 | chr22 | 45016649 | 45016747 | Heart Cardiomyocytes | 0.57 | 0.12 | intron | LINC00229 | M |
| 3267 | chr16 | 4010758 | 4011162 | Heart Cardiomyocytes | 0.77 | 0.09 | Intergenic | CREBBP | M |
| 3268 | chr13 | 35324583 | 35324774 | Heart Cardiomyocytes | 0.78 | 0.06 | Intergenic | LINC00457 | M |
| 3269 | chr9 | 821886453 | 82188827 | Heart Cardiomyocytes | 0.77 | 0.07 | intron | TLE4 | M |
| 3270 | chr11 | 3877391 | 3877608 | Heart Cardiomyocytes | 0.7 | 0.02 | promoter-TSS, TTS | STIM1, MIR4687 | M |
| 3271 | chr14 | 76795912 | 76796254 | Heart Cardiomyocytes | 0.76 | 0.1 | Intergenic | ESRRB | M |
| 3272 | chr11 | 125133120 | 125133568 | Heart Cardiomyocytes | 0.7 | 0.08 | intron | PKNOX2 | M |
| 3273 | chr6 | 12638413 | 12638585 | Heart Cardiomyocytes | 0.66 | 0.06 | Intergenic | PHACTR1 | M |
| 3274 | chr1 | 157090576 | 167090692 | Heart Cardiomyocytes | 0.67 | 0.08 | intron | DUSP27 | M |
| 3275 | chr17 | 45331940 | 45332089 | Heart Cardiomyocytes | 0.67 | 0.1 | intron | ITGB3 | M |
| 3276 | chr6 | 53517075 | 53517166 | Heart Cardiomyocytes | 0.57 | 0.04 | exon, intron | KLHL31, KLHL31 | M |
| 3277 | chr8 | 23560853 | 23561134 | Heart Cardiomyocytes | 0.61 | 0.08 | intron | NKX2-6 | M |
| 3278 | chr11 | 47587672 | 47587766 | Heart Cardiomyocytes | 0.55 | 0.04 | exon | PTPMT1 | M |
| 3279 | chr18 | 45934528 | 45934700 | Heart Cardiomyocytes | 0.55 | 0.08 | Intergenic | CTIF | M |
| 3280 | chr2 | 236526611 | 236526801 | Heart Fibroblasts | 0.04 | 0.92 | intron | AGAP1 | U |
| 3281 | chr1 | 20355843 | 20355899 | Heart Fibroblasts | 0.05 | 0.92 | Intergenic | PLA2G5 | U |
| 3282 | chr16 | 79173770 | 79174090 | Heart Fibroblasts | 0.08 | 0.92 | intron | WWOX | U |
| 3283 | chr17 | 10810563 | 10810873 | Heart Fibroblasts | 0.08 | 0.89 | Intergenic | PIRT | U |
| 3284 | chr16 | 55010895 | 55010996 | Heart Fibroblasts | 0.11 | 0.91 | Intergenic | IRX5 | U |
| 3285 | chr16 | 79174152 | 79174282 | Heart Fibroblasts | 0.12 | 0.92 | intron | WWOX | U |
| 3286 | chr13 | 47760131 | 47760254 | Heart Fibroblasts | 0.05 | 0.85 | Intergenic | HTR2A | U |
| 3287 | chr21 | 41033887 | 41034082 | Heart Fibroblasts | 0.15 | 0.93 | exon | B3GALT5 | U |
| 3288 | chr8 | 49110226 | 49110401 | Heart Fibroblasts | 0.07 | 0.83 | Intergenic | UBE2V2 | U |
| 3289 | chr7 | 104544631 | 104544863 | Heart Fibroblasts | 0.09 | 0.85 | intron | LHFPL3-AS2 | U |
| 3290 | chr8 | 13295679 | 13295959 | Heart Fibroblasts | 0.09 | 0.84 | intron | DLC1 | U |
| 3291 | chr15 | 45622883 | 45623238 | Heart Fibroblasts | 0.16 | 0.91 | Intergenic | GATM-AS1 | U |
| 3292 | chr3 | 64580263 | 64580714 | Heart Fibroblasts | 0.16 | 0.9 | exor | ADAMTS9 | U |
| 3293 | chr13 | 38955429 | 38955900 | Heart Fibroblasts | 0.1 | 0.83 | Intergenic | UFM1 | U |
| 3294 | chr1 | 1982376363 | 198237873 | Heart Fibroblasts | 0.24 | 0.95 | intron | NEK7 | U |
| 3295 | chr18 | 32893374 | 32893832 | Heart Fibroblasts | 0.22 | 0.92 | Intergenic | ZNF271 | U |
| 3296 | chr6 | 169497716 | 169498021 | Heart Fibroblasts | 0.13 | 0.82 | Intergenic | THBS2 | U |
| 3297 | chr8 | 25626261 | 25626364 | Heart Fibroblasts | 0.23 | 0.91 | Intergenic | EBF2 | U |
| 3298 | chr7 | 35569892 | 35570085 | Heart Fibroblasts | 0.24 | 0.91 | Intergenic | HERPUD2 | U |
| 3299 | chr16 | 940791 | 941094 | Heart Fibroblasts | 0.22 | 0.88 | intron | LMF1 | U |
| 3300 | chr19 | 3012448 | 3012864 | Heart Fibroblasts | 0.25 | 0.9 | intron | TLE2 | U |
| 3301 | chr6 | 169568431 | 169568565 | Heart Fibroblasts | 0.01 | 0.93 | Intergenic | THBS2 | U |
| 3302 | chr6 | 169568591 | 169568771 | Heart Fibroblasts | 0.01 | 0.88 | Intergenic | THBS2 | U |
| 3303 | chr21 | 27223171 | 27223269 | Heart Fibroblasts | 0.07 | 0.91 | Intergenic | ATP5J | U |
| 3304 | chr13 | 23013641 | 23013942 | Heart Fibroblasts | 0.08 | 0.89 | Intergenic | BASP1P1 | U |
| 3305 | chr9 | 136576958 | 136577013 | Heart Fibroblasts | 0.08 | 0.88 | intron | SARDH | U |
| 3306 | chr5 | 147854029 | 147854191 | Heart Fibroblasts | 0.08 | 0.88 | intron | HTR4 | U |
| 3307 | chr17 | 34278491 | 34278667 | Heart Fibroblasts | 0.1 | 0.9 | Intergenic | LYZL6 | U |
| 3308 | chr9 | 136576746 | 136576932 | Heart Fibroblasts | 0.08 | 0.88 | intron | SARDH | U |
| 3309 | chr5 | 160367907 | 160368132 | Heart Fibroblasts | 0.1 | 0.9 | Intergenic | LOC285629 | U |
| 3310 | chr1 | 12726365 | 12726621 | Heart Fibroblasts | 0.07 | 0.87 | exor | AADACL4 | U |
| 3311 | chr3 | 176304417 | 176304807 | Heart Fibroblasts | 0.07 | 0.87 | Intergenic | FBL1XR1 | U |
| 3312 | chr4 | 144518334 | 144518488 | Heart Fibroblasts | 0.08 | 0.87 | intron | FREM3 | U |
| 3313 | chr11 | 75984963 | 75985221 | Heart Fibroblasts | 0.1 | 0.88 | Intergenic | WNT11 | U |
| 3314 | chr10 | 100600096 | 100600434 | Heart Fibroblasts | 0.06 | 0.84 | intron | HPSE2 | U |
| 3315 | chr10 | 14745177 | 14745626 | Heart Fibroblasts | 0.14 | 0.92 | intron | FAM107B | U |
| 3316 | chr15 | 479332631 | 47933342 | Heart Fibroblasts | 0.09 | 0.86 | intron | SEMA6D | U |
| 3317 | chr4 | 25067589 | 25068017 | Heart Fibroblasts | 0.12 | 0.89 | Intergenic | LGI2 | U |
| 3318 | chr5 | 8232971 | 8233136 | Heart Fibroblasts | 0.07 | 0.83 | Intergenic | LOC729506 | U |
| 3319 | chr12 | 104750545 | 104750720 | Heart Fibroblasts | 0.14 | 0.9 | Intergenic | EID3 | U |
| 3320 | chr2 | 236845416 | 236845766 | Heart Fibroblasts | 0.12 | 0.88 | intron | AGAP1 | U |
| 3321 | chr10 | 108807125 | 108807567 | Heart Fibroblasts | 0.1 | 0.86 | intron | GORCS1 | U |
| 3322 | chr2 | 33461893 | 33462341 | Heart Fibroblasts | 0.11 | 0.87 | intron | LTBP1 | U |
| 3323 | chr2 | 3045032 | 3045115 | Heart Fibroblasts | 0.14 | 0.89 | Intergenic | TSSC1 | U |
| 3324 | chr2 | 129096704 | 129096789 | Heart Fibroblasts | 0.14 | 0.89 | Intergenic | HS6ST1 | U |
| 3325 | chr5 | 141683140 | 141683245 | Heart Fibroblasts | 0.13 | 0.87 | Intergenic | SPRY4 | U |
| 3326 | chr10 | 36381288 | 36381412 | Heart Fibroblasts | 0.17 | 0.91 | Intergenic | FZD8 | U |
| 3327 | chr6 | 159807233 | 159807380 | Heart Fibroblasts | 0.15 | 0.89 | Intergenic | FNDC1 | U |
| 3328 | chr16 | 72939959 | 72940108 | Heart Fibroblasts | 0.2 | 0.94 | intron | ZFHX3 | U |
| 3329 | chr10 | 102188822 | 102189059 | Heart Fibroblasts | 0.11 | 0.85 | Intergenic | WNT8B | U |
| 3330 | chr9 | 136577116 | 136577416 | Heart Fibroblasts | 0.09 | 0.83 | intron | SARDH | U |
| 3331 | chr1 | 61898568 | 61898890 | Heart Fibroblasts | 0.14 | 0.88 | intron | NFIA | U |
| 3332 | chr13 | 26556444 | 26556812 | Heart Fibroblasts | 0.1 | 0.84 | intron | ATP8A2 | U |
| 3333 | chr7 | 4038381 | 4038774 | Heart Fibroblasts | 0.19 | 0.93 | intron | SDK1 | U |
| 3334 | chr21 | 28838198 | 28838475 | Heart Fibroblasts | 0.16 | 0.89 | intron | MIR5009 | U |
| 3335 | chr3 | 108214631 | 108214942 | Heart Fibroblasts | 0.11 | 0.84 | intron | MYH15 | U |
| 3336 | chr2 | 204891253 | 204891624 | Heart Fibroblasts | 0.16 | 0.88 | Intergenic | ICOS | U |
| 3337 | chr14 | 80778042 | 80778428 | Heart Fibroblasts | 0.14 | 0.86 | intron | DIO2-AS1 | U |
| 3338 | chr17 | 70290476 | 70290970 | Heart Fibroblasts | 0.16 | 0.88 | Intergenic | SOX9 | U |
| 3339 | chr5 | 40914883 | 40915035 | Heart Fibroblasts | 0.17 | 0.88 | intron | C7 | U |
| 3340 | chr17 | 4510524 | 4510598 | Heart Fibroblasts | 0.2 | 0.9 | intron | SMTNL2 | U |
| 3341 | chr6 | 160683680 | 160683829 | Heart Fibroblasts | 0.14 | 0.84 | Intergenic | SLC22A2 | U |
| 3342 | chr7 | 70096458 | 70096640 | Heart Fibroblasts | 0.16 | 0.86 | intron | AUTS2 | U |
| 3343 | chr18 | 49667627 | 49667813 | Heart Fibroblasts | 0.15 | 0.85 | Intergenic | DCC | U |
| 3344 | chr4 | 184849937 | 184850214 | Heart Fibroblasts | 0.23 | 0.93 | intron | STOX2 | U |
| 3345 | chr17 | 3671806 | 3671944 | Heart Fibroblasts | 0.22 | 0.91 | intron | ITGAE | U |
| 3346 | chr10 | 34336290 | 34336713 | Heart Fibroblasts | 0.21 | 0.9 | Intergenic | LINC00838 | U |
| 3347 | chr5 | 81158936 | 81159388 | Heart Fibroblasts | 0.19 | 0.88 | Intergenic | BCKDHB | U |
| 3348 | chr5 | 17781368 | 177813727 | Heart Fibroblasts | 0.19 | 0.87 | intron | COL23A1 | U |
| 3349 | chr2 | 237572975 | 237573156 | Heart Fibroblasts | 0.2 | 0.88 | Intergenic | CXCR7 | U |
| 3350 | chr12 | 15236157 | 15236350 | Heart Fibroblasts | 0.16 | 0.84 | Intergenic | PDE6H | U |
| 3351 | chr7 | 94647863 | 94648116 | Heart Fibroblasts | 0.23 | 0.91 | intron | PPP1R9A | U |
| 3352 | chr10 | 60697460 | 60697760 | Heart Fibroblasts | 0.14 | 0.82 | Intergenic | FAM133CP | U |
| 3353 | chr18 | 19996170 | 19996646 | Heart Fibroblasts | 0.21 | 0.89 | exon | CTAGE1 | U |
| 3354 | chrX | 1181626353 | 118162689 | Heart Fibroblasts | 0.14 | 0.81 | Intergenic | LONRF3 | U |
| 3355 | chr8 | 1318016 | 1318077 | Heart Fibroblasts | 0.21 | 0.88 | Intergenic | LOC286083 | U |
| 3356 | chr1 | 2351586963 | 235158809 | Heart Fibroblasts | 0.21 | 0.88 | Intergenic | SNORA14B | U |
| 3357 | chr4 | 4341969 | 4342235 | Heart Fibroblasts | 0.13 | 0.8 | Intergenic | NSG1 | U |
| 3358 | chr21 | 47349563 | 47349788 | Heart Fibroblasts | 0.26 | 0.92 | intron | PCBP3 | U |
| 3359 | chr6 | 126381299 | 126381737 | Heart Fibroblasts | 0.25 | 0.91 | Intergenic | MIR5695 | U |
| 3360 | chr3 | 99359629 | 99360088 | Heart Fibroblasts | 0.22 | 0.88 | intron | COL8A1 | U |
| 3361 | chr11 | 2001208 | 2001355 | Heart Fibroblasts | 0.25 | 0.9 | Intergenic | MRPL23-AS1 | U |
| 3362 | chr17 | 74365585 | 74365762 | Heart Fibroblasts | 0.24 | 0.89 | Intergenic | SPHK1 | U |
| 3363 | chr2 | 107345065 | 107345421 | Heart Fibroblasts | 0.23 | 0.88 | Intergenic | ST6GAL2 | U |
| 3364 | chr11 | 78509223 | 78509392 | Heart Fibroblasts | 0.23 | 0.87 | intron | TENM4 | U |
| 3365 | chr10 | 45589779 | 45590124 | Heart Fibroblasts | 0.24 | 0.88 | Intergenic | RSU1P2 | U |
| 3366 | chr7 | 105659527 | 105659911 | Heart Fibroblasts | 0.25 | 0.89 | intron | CDHR3 | U |
| 3367 | chr17 | 67893232 | 67893395 | Heart Fibroblasts | 0.25 | 0.88 | Intergenic | KCNJ16 | U |
| 3368 | chr4 | 79038448 | 79038812 | Heart Fibroblasts | 0.26 | 0.89 | intron | FRAS1 | U |
| 3369 | chr2 | 66529058 | 66529160 | Heart Fibroblasts | 0.3 | 0.92 | Intergenic | MIR4778 | U |
| 3370 | chr17 | 2885214 | 2885369 | Heart Fibroblasts | 0.29 | 0.91 | intron | RAP1GAP2 | U |
| 3371 | chr11 | 120701316 | 120701516 | Heart Fibroblasts | 0.23 | 0.85 | intron | GRIKA | U |
| 3372 | chr5 | 142481323 | 142481751 | Heart Fibroblasts | 0.25 | 0.87 | intron | ARHGAP26 | U |
| 3373 | chr6 | 145357601 | 145358045 | Heart Fibroblasts | 0.26 | 0.88 | Intergenic | LOC100507557 | U |
| 3374 | chr2 | 2060527743 | 206053228 | Heart Fibroblasts | 0.29 | 0.91 | intron | PARD38 | U |
| 3375 | chr12 | 104750358 | 104750482 | Heart Fibroblasts | 0.22 | 0.83 | Intergenic | EID3 | U |
| 3376 | chr12 | 114652758 | 114652961 | Heart Fibroblasts | 0.22 | 0.83 | Intergenic | TBX5 | U |
| 3377 | chr16 | 11760791 | 11761065 | Heart Fibroblasts | 0.23 | 0.84 | Intergenic | SNN | U |
| 3378 | chr3 | 150124366 | 150124717 | Heart Fibroblasts | 0.26 | 0.87 | Intergenic | TSC22D2 | U |
| 3379 | chr11 | 1180410691 | 118041476 | Heart Fibroblasts | 0.23 | 0.84 | intron | SCN2B | U |
| 3380 | chr7 | 414870911 | 41487178 | Heart Fibroblasts | 0.32 | 0.92 | Intergenic | INHBA-AS1 | U |
| 3381 | chr21 | 44566045 | 44566149 | Heart Fibroblasts | 0.29 | 0.89 | Intergenic | CRYAA | U |
| 3382 | chr1 | 92055516 | 92055639 | Heart Fibroblasts | 0.26 | 0.86 | Intergenic | CDC7 | U |
| 3383 | chr2 | 1742576231 | 174257782 | Heart Fibroblasts | 0.24 | 0.83 | Intergenic | CDCA7 | U |
| 3384 | chr21 | 37819075 | 37819258 | Heart Fibroblasts | 0.32 | 0.91 | Intergenic | CLDN14 | U |
| 3385 | chr5 | 177955938 | 177956081 | Heart Fibroblasts | 0.28 | 0.86 | intron | COL23A1 | U |
| 3386 | chr2 | 42140447 | 42140683 | Heart Fibroblasts | 0.3 | 0.88 | Intergenic | LOC388942 | U |
| 3387 | chr12 | 111183872 | 111184194 | Heart Fibroblasts | 0.33 | 0.91 | Intergenic | PPPICC | U |
| 3388 | chr11 | 126187044 | 126187158 | Heart Fibroblasts | 0.36 | 0.93 | intron | DCPS | U |
| 3389 | chr4 | 156416292 | 156416774 | Heart Fibroblasts | 0.3 | 0.87 | Intergenic | MAP9 | U |
| 3390 | chr10 | 30547931 | 30548270 | Heart Fibroblasts | 0.28 | 0.84 | Intergenic | MTPAP | U |
| 3391 | chr1 | 7009315 | 7009624 | Heart Fibroblasts | 0.3 | 0.85 | intron | CAMTA1 | U |
| 3392 | chr18 | 2327465 | 2327567 | Heart Fibroblasts | 0.37 | 0.91 | Intergenic | METTL4 | U |
| 3393 | chr13 | 109637815 | 109638067 | Heart Fibroblasts | 0.38 | 0.92 | intron | MYO16 | U |
| 3394 | chr19 | 556387543 | 55638994 | Heart Fibroblasts | 0.35 | 0.88 | Intergenic | PPP1R12C | U |
| 3395 | chr20 | 54316572 | 54316673 | Heart Fibroblasts | 0.19 | 0.9 | Intergenic | CBLN4 | U |
| 3396 | chr3 | 89620409 | 89620655 | Heart Fibroblasts | 0.2 | 0.83 | Intergenic | EPHA3 | U |
| 3397 | chr6 | 1695677771 | 169567965 | Heart Fibroblasts | 0.05 | 0.9 | Intergenic | THBS2 | U |
| 3398 | chr8 | 140847540 | 140847721 | Heart Fibroblasts | 0.09 | 0.93 | intron | TRAPPC9 | U |
| 3399 | chr6 | 1695688593 | 169569139 | Heart Fibroblasts | 0.06 | 0.89 | Intergenic | THBS2 | U |
| 3400 | chr20 | 11969797 | 11969965 | Heart Fibroblasts | 0.11 | 0.91 | Intergenic | BTBD3 | U |
| 3401 | chr5 | 177793999 | 177794281 | Heart Fibroblasts | 0.03 | 0.89 | intron | COL23A1 | U |
| 3402 | chr14 | 35727869 | 35728029 | Heart Fibroblasts | 0.06 | 0.86 | intron | KIAA0391 | U |
| 3403 | chr9 | 78903902 | 78904297 | Heart Fibroblasts | 0.18 | 0.91 | intron | PCSK5 | U |
| 3404 | chr7 | 140267969 | 140268338 | Heart Fibroblasts | 0.15 | 0.87 | intron | DENND2A | U |
| 3405 | chr12 | 133008884 | 133009120 | Heart Fibroblasts | 0.2 | 0.89 | Intergenic | FBRSLJ | U |
| 3406 | chr6 | 14328472 | 14328888 | Heart Fibroblasts | 0.18 | 0.87 | Intergenic | CD83 | U |
| 3407 | chr5 | 172704461 | 172704671 | Heart Fibroblasts | 0.21 | 0.84 | Intergenic | NKX2-5 | U |
| 3408 | chr12 | 1148363683 | 114836517 | Heart Fibroblasts | 0.92 | 0.16 | exon | TBX5 | M |
| 3409 | chr12 | 114836605 | 114836934 | Heart Fibroblasts | 0.91 | 0.15 | intron | TBX5 | M |
| 3410 | chr12 | 1148524423 | 114852769 | Heart Fibroblasts | 0.85 | 0.15 | Intergenic | TBX5-AS1 | M |
| 3411 | chr4 | 549680453 | 54968482 | Heart Fibroblasts | 0.84 | 0.16 | TTS | GSX2 | M |
| 3412 | chr11 | 32459877 | 32460164 | Heart Fibroblasts | 0.62 | 0.1 | exon | WT1-AS | M |
| 3413 | chr7 | 32109909 | 32110030 | Heart Fibroblasts | 0.63 | 0.12 | exon | PDE1C | M |
| 3414 | chr6 | 85480636 | 85481113 | Heart Fibroblasts | 0.64 | 0.16 | Intergenic | TBX18 | M |
| 3415 | chr11 | 32449725 | 32449861 | Heart Fibroblasts | 0.9 | 0.14 | intron | WT1 | M |
| 3416 | chr11 | 32450215 | 32450498 | Heart Fibroblasts | 0.8 | 0.12 | intron | WT1 | M |
| 3417 | chr1 | 119527415 | 119527583 | Heart Fibroblasts | 0.84 | 0.1 | intron | TBX15 | M |
| 3418 | chr8 | 259070763 | 25907377 | Heart Fibroblasts | 0.85 | 0.12 | Intergenic | BF2 | M |
| 3419 | chr1 | 119527674 | 119527790 | Heart Fibroblasts | 0.83 | 0.11 | intron | TBX15 | M |
| 3420 | chr1 | 119531998 | 119532085 | Heart Fibroblasts | 0.85 | 0.17 | exon | TBX15 | M |
| 3421 | chr6 | 85476760 | 85476892 | Heart Fibroblasts | 0.85 | 0.17 | Intergenic | TBX18 | M |
| 3422 | chr8 | 25906732 | 25906809 | Heart Fibroblasts | 0.73 | 0.1 | Intergenic | EBF2 | M |
| 3423 | chr1 | 119526837 | 119526992 | Heart Fibroblasts | 0.74 | 0.12 | intron | TBX15 | M |
| 3424 | chr4 | 174452563 | 174452691 | Heart Fibroblasts | 0.8 | 0.2 | intron | NBLA00301 | M |
| 3425 | chr1 | 119531832 | 119531951 | Heart Fibroblasts | 0.73 | 0.14 | intron | TBX15 | M |
| 3426 | chr1 | 197890565 | 197890662 | Heart Fibroblasts | 0.78 | 0.2 | exon | LHX9 | M |
| 3427 | chr1 | 119526993 | 119527410 | Heart Fibroblasts | 0.63 | 0.06 | intron | TBX15 | M |
| 3428 | chr4 | 174452381 | 174452507 | Heart Fibroblasts | 0.7 | 0.14 | intron | NBLA00301 | M |
| 3429 | chr1 | 119531625 | 119531709 | Heart Fibroblasts | 0.7 | 0.15 | intron | TBX15 | M |
| 3430 | chr1 | 119521979 | 119522426 | Heart Fibroblasts | 0.66 | 0.13 | intron | TBX15 | M |
| 3431 | chr15 | 51633704 | 51633873 | Heart Fibroblasts | 0.59 | 0.09 | promoter-TSS | GLDN | M |
| 3432 | chr4 | 1744521683 | 174452256 | Heart Fibroblasts | 0.57 | 0.13 | promoter-TSS | HAND2 | M |
| 3433 | chr5 | 760142561 | 76014547 | Vascular Endothelial cells | 0.16 | 0.88 | intron | F2R | U |
| 3434 | chr17 | 80804200 | 80804265 | Vascular Endothelial cells | 0.16 | 0.85 | intron | TBCD | U |
| 3435 | chr17 | 8214564 | 8214718 | Vascular Endothelial cells | 0.19 | 0.87 | intron | ARHGEF15 | U |
| 3436 | chr5 | 778199551 | 77820339 | Vascular Endothelial cells | 0.24 | 0.91 | intron | LHFPL2 | U |
| 3437 | chr2 | 128416959 | 128417047 | Vascular Endothelial cells | 0.27 | 0.92 | intron | LIMS2 | U |
| 3438 | chr5 | 32098278 | 32098495 | Vascular Endothelial cells | 0.28 | 0.93 | intron | PDZD2 | U |
| 3439 | chr16 | 1562267 | 1562660 | Vascular Endothelial cells | 0.21 | 0.86 | intron | IFT140 | U |
| 3440 | chr5 | 71853664 | 71853918 | Vascular Endothelial cells | 0.18 | 0.83 | Intergenic | ZNF366 | U |
| 3441 | chr19 | 11707038 | 11707246 | Vascular Endothelial cells | 0.27 | 0.91 | Intergenic, promote [symbol] | ZNF627 | U |
| 3442 | chr1 | 1853364171 | 185336695 | Vascular Endothelial cells | 0.25 | 0.89 | Intergenic | LOC100288079 | U |
| 3443 | chr20 | 3719956 | 3720042 | Vascular Endothelial cells | 0.29 | 0.92 | intron | HSPA12B | U |
| 3444 | chr11 | 65379744 | 65379962 | Vascular Endothelial cells | 0.19 | 0.81 | intron | MAP3K11 | U |
| 3445 | chr2 | 160081735 | 160082056 | Vascular Endothelial cells | 0.29 | 0.91 | intron | TANC1 | U |
| 3446 | chr6 | 113817584 | 113817951 | Vascular Endothelial cells | 0.26 | 0.88 | Intergenic | MARCKS | U |
| 3447 | chr2 | 218848041 | 218848444 | Vascular Endothelial cells | 0.29 | 0.9 | Intergenic | TNS1 | U |
| 3448 | chr12 | 50040684 | 50040779 | Vascular Endothelial cells | 0.28 | 0.89 | exon | FMNL3 | U |
| 3449 | chr14 | 104326499 | 104326617 | Vascular Endothelial cells | 0.25 | 0.86 | Intergenic | LINC00637 | U |
| 3450 | chr3 | 1293267561 | 129326961 | Vascular Endothelial cells | 0.25 | 0.86 | Intergenic | PLXND1 | U |
| 3451 | chr16 | 87213128 | 87213220 | Vascular Endothelial cells | 0.26 | 0.86 | Intergenic | C16orf95 | U |
| 3452 | chr20 | 4101440 | 4101638 | Vascular Endothelial cells | 0.27 | 0.86 | Intergenic | SMOX | U |
| 3453 | chr11 | 109900473 | 109900818 | Vascular Endothelial cells | 0.28 | 0.87 | Intergenic | ZC3H12C | U |
| 3454 | chr5 | 171707601 | 17171195 | Vascular Endothelial cells | 0.28 | 0.85 | intron | LOC285696 | U |
| 3455 | chr9 | 138903401 | 138903478 | Vascular Endothelial cells | 0.25 | 0.78 | exon | NACC2 | U |
| 3456 | chr7 | 45197792 | 45197980 | Vascular Endothelial cells | 0.24 | 0.75 | intron | RAMP3 | U |
| 3457 | chr9 | 1306139863 | 130614101 | Vascular Endothelial cells | 0.15 | 0.86 | intron | ENG | U |
| 3458 | chr1 | 21605850 | 21605900 | Vascular Endothelial cells | 0.18 | 0.88 | exon | ECE1 | U |
| 3459 | chr16 | 1562673 | 1563151 | Vascular Endothelial cells | 0.2 | 0.89 | intron | IFT140 | U |
| 3460 | chr4 | 166687299 | 166687454 | Vascular Endothelial cells | 0.22 | 0.89 | Intergenic | TLL1 | U |
| 3461 | chr2 | 48613218 | 48613599 | Vascular Endothelial cells | 0.18 | 0.85 | Intergenic | PPP1R21 | U |
| 3462 | chr9 | 132339217 | 132339270 | Vascular Endothelial cells | 0.24 | 0.89 | Intergenic | C9orf50 | U |
| 3463 | chr21 | 44899070 | 44899170 | Vascular Endothelial cells | 0.21 | 0.85 | promoter-TSS, Interg [symbol] | LINC00313, LINC0031 [symbol] | U |
| 3464 | chr1 | 51320582 | 51320733 | Vascular Endothelial cells | 0.29 | 0.93 | intron | FAF1 | U |
| 3465 | chr1 | 185336511 | 185336696 | Vascular Endothelial cells | 0.23 | 0.87 | Intergenic | LOC100288079 | U |
| 3466 | chr9 | 1305307613 | 130531143 | Vascular Endothelial cells | 0.2 | 0.84 | intron | SH2D3C | U |
| 3467 | chr17 | 465503 | 465626 | Vascular Endothelial cells | 0.24 | 0.87 | intron | VPS53 | U |
| 3468 | chr10 | 30140200 | 30140383 | Vascular Endothelial cells | 0.22 | 0.85 | Intergenic | KIAA1462 | U |
| 3469 | chr7 | 139573546 | 139573948 | Vascular Endothelial cells | 0.24 | 0.87 | intron | TBXAS1 | U |
| 3470 | chr11 | 86662732 | 86663161 | Vascular Endothelial cells | 0.23 | 0.86 | exon | FZD4 | U |
| 3471 | chr11 | 75017440 | 75017929 | Vascular Endothelial cells | 0.25 | 0.88 | intron | ARRB | U |
| 3472 | chr21 | 467298211 | 46729883 | Vascular Endothelial cells | 0.23 | 0.85 | Intergenic | LOC642852 | U |
| 3473 | chr1 | 55097881 | 55097980 | Vascular Endothelial cells | 0.23 | 0.85 | intron | ACOT11 | U |
| 3474 | chr3 | 129214369 | 129214507 | Vascular Endothelial cells | 0.3 | 0.92 | exon | IFT122 | U |
| 3475 | chr15 | 74678214 | 74678365 | Vascular Endothelial cells | 0.25 | 0.87 | Intergenic | CYP11A1 | U |
| 3476 | chr13 | 114585785 | 114585973 | Vascular Endothelial cells | 0.25 | 0.87 | Intergenic | LOC100506394 | U |
| 3477 | chr2 | 46575075 | 46575353 | Vascular Endothelial cells | 0.22 | 0.84 | intron | EPAS1 | U |
| 3478 | chr15 | 41274883 | 41275365 | Vascular Endothelial cells | 0.24 | 0.86 | intron, exon | INO80, INO80 | U |
| 3479 | chr17 | 80560634 | 80560718 | Vascular Endothelial cells | 0.29 | 0.9 | exon | FOXK2 | U |
| 3480 | chr17 | 30051202 | 30051379 | Vascular Endothelial cells | 0.22 | 0.83 | Intergenic | COPR5 | U |
| 3481 | chr17 | 7758473 | 7758693 | Vascular Endothelial cells | 0.24 | 0.85 | promoter-TSS, TTS | TMEM88, KDM6B | U |
| 3482 | chr8 | 641451 | 641542 | Vascular Endothelial cells | 0.3 | 0.9 | intron | ERICH1 | U |
| 3483 | chr22 | 46913360 | 46913470 | Vascular Endothelial cells | 0.29 | 0.89 | intron | CELSR1 | U |
| 3484 | chr21 | 46739702 | 46739820 | Vascular Endothelial cells | 0.19 | 0.79 | Intergenic | LOC642852 | U |
| 3485 | chr6 | 42096212 | 42096342 | Vascular Endothelial cells | 0.26 | 0.86 | intron | C6orf132 | U |
| 3486 | chr7 | 47369132 | 47369289 | Vascular Endothelial cells | 0.27 | 0.87 | intron | TNS3 | U |
| 3487 | chr5 | 149844479 | 149844642 | Vascular Endothelial cells | 0.25 | 0.85 | Intergenic | RPS14 | U |
| 3488 | chr6 | 138126946 | 138127185 | Vascular Endothelial cells | 0.24 | 0.84 | Intergenic | TNFAIP3 | U |
| 3489 | chr10 | 31989512 | 31989773 | Vascular Endothelial cells | 0.29 | 0.89 | Intergenic | ARHGAP12 | U |
| 3490 | chr10 | 30444665 | 30444961 | Vascular Endothelial cells | 0.26 | 0.86 | Intergenic | KIAA1462 | U |
| 3491 | chr12 | 121527599 | 121527904 | Vascular Endothelial cells | 0.25 | 0.85 | Intergenic | P2RX7 | U |
| 3492 | chr3 | 12964529 | 12964881 | Vascular Endothelial cells | 0.27 | 0.87 | intron | IQSEC1 | U |
| 3493 | chr3 | 129320831 | 129321288 | Vascular Endothelial cells | 0.26 | 0.86 | intron | PLXND1 | U |
| 3494 | chr11 | 110268874 | 110268980 | Vascular Endothelial cells | 0.25 | 0.84 | Intergenic | FDX1 | U |
| 3495 | chr19 | 39190658 | 39190829 | Vascular Endothelial cells | 0.25 | 0.84 | intron | ACTN4 | U |
| 3496 | chr9 | 138902846 | 138903063 | Vascular Endothelial cells | 0.29 | 0.88 | exon | NACC2 | U |
| 3497 | chr14 | 102817450 | 102817718 | Vascular Endothelial cells | 0.3 | 0.89 | intron | CINP | U |
| 3498 | chr18 | 19510086 | 19510358 | Vascular Endothelial cells | 0.25 | 0.84 | Intergenic | MIR1-2 | U |
| 3499 | chr8 | 131429260 | 131429605 | Vascular Endothelial cells | 0.29 | 0.88 | intron | ASAP1 | U |
| 3500 | chr7 | 131217619 | 131217688 | Vascular Endothelial cells | 0.26 | 0.84 | intron | PODXL | U |
| 3501 | chr1 | 46087002 | 46087125 | Vascular Endothelial cells | 0.26 | 0.84 | exon | CCDC17 | U |
| 3502 | chr19 | 6219883 | 6220017 | Vascular Endothelial cells | 0.23 | 0.81 | intron | MLLT1 | U |
| 3503 | chr7 | 100041870 | 100042025 | Vascular Endothelial cells | 0.23 | 0.81 | Intergenic | PPP1R35 | U |
| 3504 | chr21 | 43445208 | 43445384 | Vascular Endothelial cells | 0.26 | 0.84 | TTS | ZNF295-AS1 | U |
| 3505 | chr2 | 20773321 | 20773646 | Vascular Endothelial cells | 0.28 | 0.86 | Intergenic | HS1BP3 | U |
| 3506 | chr12 | 19104640 | 19104983 | Vascular Endothelial cells | 0.26 | 0.84 | Intergenic | PLEKHA5 | U |
| 3507 | chr8 | 82305505 | 82305853 | Vascular Endothelial cells | 0.27 | 0.85 | Intergenic | PMP2 | U |
| 3508 | chr8 | 26032468 | 26032900 | Vascular Endothelial cells | 0.28 | 0.86 | Intergenic | PPP2R2A | U |
| 3509 | chr17 | 7758376 | 7758448 | Vascular Endothelial cells | 0.19 | 0.76 | promoter-TSS | TMEM88 | U |
| 3510 | chr5 | 3303745 | 3303868 | Vascular Endothelial cells | 0.29 | 0.86 | Intergenic | LOC285577 | U |
| 3511 | chr9 | 124420223 | 124420374 | Vascular Endothelial cells | 0.26 | 0.83 | intron | DAB2IP | U |
| 3512 | chr12 | 50041247 | 50041566 | Vascular Endothelial cells | 0.28 | 0.85 | intron, exon | FMNL3, FMNL3 | U |
| 3513 | chr4 | 41052805 | 41053155 | Vascular Endothelial cells | 0.29 | 0.86 | intron | APBB2 | U |
| 3514 | chr20 | 62319997 | 62320091 | Vascular Endothelial cells | 0.3 | 0.86 | intron | RTEL1-TNFRSF6B | U |
| 3515 | chr19 | 11364111 | 11364456 | Vascular Endothelial cells | 0.29 | 0.85 | intron | DOCK6 | U |
| 3516 | chr10 | 45631136 | 45631206 | Vascular Endothelial cells | 0.28 | 0.83 | intron | RSU1P2 | U |
| 3517 | chr10 | 30034810 | 30034889 | Vascular Endothelial cells | 0.33 | 0.88 | Intergenic | SVIL | U |
| 3518 | chr17 | 1075364 | 1075599 | Vascular Endothelial cells | 0.28 | 0.83 | intron | ABR | U |
| 3519 | chr9 | 140281552 | 140281802 | Vascular Endothelial cells | 0.25 | 0.8 | intron | EXD3 | U |
| 3520 | chr6 | 33577711 | 33578032 | Vascular Endothelial cells | 0.24 | 0.79 | Intergenic | ITPRS | U |
| 3521 | chr11 | 76377013 | 76377449 | Vascular Endothelial cells | 0.3 | 0.85 | intron | LRRC32 | U |
| 3522 | chr6 | 108773814 | 108774286 | Vascular Endothelial cells | 0.27 | 0.82 | intron | LACE1 | U |
| 3523 | chr11 | 74231820 | 74232309 | Vascular Endothelial cells | 0.29 | 0.84 | Intergenic | LIPT2 | U |
| 3524 | chr18 | 77172345 | 77172402 | Vascular Endothelial cells | 0.28 | 0.82 | intron | NFATC1 | U |
| 3525 | chr14 | 102526697 | 102526768 | Vascular Endothelial cells | 0.28 | 0.82 | Intergenic | HSP90AA1 | U |
| 3526 | chr19 | 45383231 | 45383387 | Vascular Endothelial cells | 0.24 | 0.78 | intron | PVRL2 | U |
| 3527 | chr6 | 170251667 | 170251859 | Vascular Endothelial cells | 0.27 | 0.81 | Intergenic | LINC00242 | U |
| 3528 | chr19 | 419592 | 419789 | Vascular Endothelial cells | 0.23 | 0.77 | intron | SHC2 | U |
| 3529 | chr2 | 128394957 | 128395180 | Vascular Endothelial cells | 0.29 | 0.83 | TTS | LIMS2 | U |
| 3530 | chr7 | 739312 | 739563 | Vascular Endothelial cells | 0.29 | 0.83 | intron | PRKAR1B | U |
| 3531 | chr16 | 78704128 | 78704380 | Vascular Endothelial cells | 0.31 | 0.85 | intron | WWOX | U |
| 3532 | chr7 | 158299886 | 158300208 | Vascular Endothelial cells | 0.3 | 0.84 | intron | PTPRN2 | U |
| 3533 | chr21 | 41934629 | 41934846 | Vascular Endothelial cells | 0.31 | 0.84 | intron | DSCAM | U |
| 3534 | chr2 | 127819304 | 127819650 | Vascular Endothelial cells | 0.27 | 0.8 | intron | BIN1 | U |
| 3535 | chr8 | 144286474 | 144286703 | Vascular Endothelial cells | 0.29 | 0.81 | Intergenic | GPIHBP1 | U |
| 3536 | chr19 | 42005720 | 42005973 | Vascular Endothelial cells | 0.34 | 0.86 | intron | LOC100505495 | U |
| 3537 | chr20 | 61509056 | 61509139 | Vascular Endothelial cells | 0.28 | 0.79 | TTS | DIDO1 | U |
| 3538 | chr1 | 230673463 | 230673575 | Vascular Endothelial cells | 0.3 | 0.81 | Intergenic | COG2 | U |
| 3539 | chr14 | 103702488 | 103702654 | Vascular Endothelial cells | 0.32 | 0.83 | Intergenic | LINC00605 | U |
| 3540 | chr9 | 139640201 | 139640430 | Vascular Endothelial cells | 0.3 | 0.81 | promoter-TSS | LOC100128593 | U |
| 3541 | chr9 | 140282141 | 140282234 | Vascular Endothelial cells | 0.35 | 0.85 | intron | EXD3 | U |
| 3542 | chr22 | 47559806 | 47559920 | Vascular Endothelial cells | 0.3 | 0.8 | intron | TBC1D22A | U |
| 3543 | chrX | 7816429 | 7816561 | Vascular Endothelial cells | 0.24 | 0.74 | Intergenic | VCX | U |
| 3544 | chr3 | 197192159 | 197192336 | Vascular Endothelial cells | 0.34 | 0.84 | Intergenic | BDH1 | U |
| 3545 | chr17 | 41167472 | 41167708 | Vascular Endothelial cells | 0.31 | 0.81 | TTS, exon | IFI35, VAT1 | U |
| 3546 | chr4 | 39392193 | 39392289 | Vascular Endothelial cells | 0.28 | 0.76 | Intergenic | KL8 | U |
| 3547 | chr14 | 103750028 | 103750123 | Vascular Endothelial cells | 0.35 | 0.81 | Intergenic | EIF5 | U |
| 3548 | chr8 | 49595474 | 49595652 | Vascular Endothelial cells | 0.24 | 0.84 | Intergenic | EFCAB1 | U |
| 3549 | chr3 | 149333935 | 149334377 | Vascular Endothelial cells | 0.28 | 0.84 | intron | WWTR1 | U |
| 3550 | chr3 | 50371010 | 50371191 | Vascular Endothelial cells | 0.3 | 0.83 | intron | RASSF1 | U |
| 3551 | chr14 | 35883185 | 35883512 | Vascular Endothelial cells | 0.27 | 0.89 | Intergenic | NFKBIA | U |
| 3552 | chr9 | 116265681 | 116266124 | Vascular Endothelial cells | 0.2 | 0.89 | intron | RGS3 | U |
| 3553 | chr22 | 22112353 | 22112706 | Vascular Endothelial cells | 0.27 | 0.91 | Intergenic | YPEL1 | U |
| 3554 | chr22 | 32727342 | 32727399 | Vascular Endothelial cells | 0.25 | 0.82 | Intergenic | RFPL3 | U |
| 3555 | chr22 | 41626840 | 41627156 | Vascular Endothelial cells | 0.28 | 0.85 | exon | L3MBTL2 | U |
| 3556 | chr20 | 38024079 | 38024417 | Vascular Endothelial cells | 0.28 | 0.84 | Intergenic | LOC339568 | U |
| 3557 | chr14 | 54016534 | 54016806 | Vascular Endothelial cells | 0.29 | 0.84 | Intergenic | DDHD1 | U |
| 3558 | chr1 | 212415240 | 212415615 | Vascular Endothelial cells | 0.31 | 0.82 | Intergenic | PPP2R5A | U |
| 3559 | chr20 | 36763521 | 36763771 | Vascular Endothelial cells | 0.29 | 0.79 | intron | TGM2 | U |
| 3560 | chr15 | 41219177 | 41219309 | Vascular Endothelial cells | 0.8 | 0.2 | Intergenic | DLL4 | M |
| 3561 | chr5 | 10288725 | 10288850 | Vascular Endothelial cells | 0.73 | 0.14 | intron | CMBL | M |
| 3562 | chr16 | 75142901 | 75143153 | Vascular Endothelial cells | 0.67 | 0.08 | exon | ZNRF1 | M |
| 3563 | chr19 | 46094335 | 46094504 | Vascular Endothelial cells | 0.65 | 0.12 | exon | GPR4 | M |
| 3564 | chr1 | 6535629 | 6535666 | Vascular Endothelial cells | 0.67 | 0.15 | intron | PLEKHG5 | M |
| 3565 | chr19 | 8398351 | 8398591 | Vascular Endothelial cells | 0.68 | 0.16 | intron, exon | KANK3, KANK3 | M |
| 3566 | chr15 | 90728654 | 90728847 | Vascular Endothelial cells | 0.63 | 0.13 | intron | SEMA4B | M |
| 3567 | chr3 | 129063124 | 129063271 | Vascular Endothelial cells | 0.61 | 0.13 | Intergenic | H1FX | M |
| 3568 | chr19 | 8399168 | 399335 | Vascular Endothelial cells | 0.7 | 0.23 | exon, intron | KANK3, KANK3 | M |
| 3569 | chr19 | 46094508 | 46094547 | Vascular Endothelial cells | 0.63 | 0.21 | exon | GPR4 | M |
| 3570 | chr3 | 193922356 | 193922482 | Vascular Endothelial cells | 0.59 | 0.17 | Intergenic | HES1 | M |
| 3571 | chr8 | 49782523 | 49782603 | Vascular Endothelial cells | 0.63 | 0.24 | Intergenic | SNAI2 | M |
| 3572 | chr17 | 65242623 | 65242741 | Vascular Endothelial cells | 0.58 | 0.2 | Intergenic | HELZ | M |
| 3573 | chr1 | 24119962 | 24120129 | Vascular Endothelial cells | 0.61 | 0.23 | intron | LYPLA2 | M |
| 3574 | chr7 | 150598713 | 150598926 | Vascular Endothelial cells | 0.56 | 0.18 | Intergenic | ABP1 | M |
| 3575 | chr2 | 10261227 | 10261460 | Vascular Endothelial cells | 0.59 | 0.22 | Intergenic | RRM2 | M |
| 3576 | chr19 | 55997002 | 55997101 | Vascular Endothelial cells | 0.66 | 0.3 | intron | NAT14 | M |
| 3577 | chr2 | 241519290 | 241519427 | Vascular Endothelial cells | 0.62 | 0.27 | Intergenic | CAPN10 | M |
| 3578 | chr17 | 37828205 | 37828378 | Vascular Endothelial cells | 0.57 | 0.22 | exon | PGAP3 | M |
| 3579 | chrX | 149529520 | 149529625 | Vascular Endothelial cells | 0.59 | 0.26 | Intergenic | MAMLD1 | M |
| 3580 | chr6 | 150276493 | 150276716 | Vascular Endothelial cells | 0.61 | 0.19 | Intergenic | ULBP1 | M |
| 3581 | chr19 | 8399343 | 8399484 | Vascular Endothelial cells | 0.68 | 0.1 | exon | KANK3 | M |
| 3582 | chr6 | 6002647 | 6002703 | Vascular Endothelial cells | 0.69 | 0.24 | exon | NRN1 | M |
| 3583 | chr17 | 47296997 | 47297158 | Vascular Endothelial cells | 0.64 | 0.19 | exon, intron | ABI3, ABI3 | M |
| 3584 | chr22 | 24627294 | 24627458 | Vascular Endothelial cells | 0.62 | 0.3 | exon, intron | GGT5, GGT5 | M |
| 3585 | chr17 | 74419888 | 74420090 | Blood B cells | 0.03 | 0.95 | intron | UBE2O | U |
| 3586 | chr15 | 91138717 | 91138945 | Blood B cells | 0.02 | 0.94 | intron | CRTC3 | U |
| 3587 | chr19 | 16554452 | 16554747 | Blood B cells | 0.03 | 0.95 | intron | EPS15L1 | U |
| 3588 | chr15 | 50736822 | 50737206 | Blood B cells | 0.02 | 0.94 | intron | USP8 | U |
| 3589 | chr21 | 44800830 | 44801108 | Blood B cells | 0.01 | 0.91 | Intergenic | SIK1 | U |
| 3590 | chr16 | 29348723 | 29349009 | Blood B cells | 0.02 | 0.92 | intron | SNX29P2 | U |
| 3591 | chr11 | 45960244 | 45960604 | Blood B cells | 0.03 | 0.93 | intron | PHF21A | U |
| 3592 | chr4 | 38764383 | 38764513 | Blood B cells | 0.02 | 0.91 | Intergenic | TLR10 | U |
| 3593 | chr12 | 65007600 | 65007772 | Blood B cells | 0.02 | 0.91 | intron | RASSF3 | U |
| 3594 | chr16 | 71762004 | 71762092 | Blood B cells | 0.06 | 0.95 | TTS | AP1G1 | U |
| 3595 | chr16 | 85944509 | 85944670 | Blood B cells | 0.04 | 0.92 | intron | IRFB | U |
| 3596 | chr7 | 97943968 | 97944178 | Blood B cells | 0.05 | 0.92 | intron | BAIAP2L1 | U |
| 3597 | chr13 | 111328090 | 111328432 | Blood B cells | 0.09 | 0.96 | intron | CARS2 | U |
| 3598 | chr11 | 82777661 | 82777873 | Blood B cells | 0.06 | 0.92 | intron | RAB30 | U |
| 3599 | chr16 | 216579613 | 21658137 | Blood B cells | 0.09 | 0.95 | intron | IGSF6 | U |
| 3600 | chr1 | 101712282 | 101712511 | Blood B cells | 0.03 | 0.89 | Intergenic | S1PRI | U |
| 3601 | chr8 | 38134856 | 38135040 | Blood B cells | 0.1 | 0.95 | intron | WHSC1L1 | U |
| 3602 | chr12 | 131464806 | 131465103 | Blood B cells | 0.05 | 0.9 | intron | GPR133 | U |
| 3603 | chr10 | 45390700 | 45390846 | Blood B cells | 0.09 | 0.93 | intron | TMEM72-AS1 | U |
| 3604 | chr6 | 64375368 | 64375609 | Blood B cells | 0.11 | 0.93 | intron | PHF3 | U |
| 3605 | chr3 | 42705063 | 42705491 | Blood B cells | 0.11 | 0.92 | intron | ZBTB47 | U |
| 3606 | chr11 | 2414931 | 2415350 | Blood B cells | 0.12 | 0.92 | intron | CD81 | U |
| 3607 | chr12 | 57681746 | 57682066 | Blood B cells | 0.16 | 0.94 | intron | R3HDM2 | U |
| 3608 | chr6 | 107096723 | 107097077 | Blood B cells | 0.02 | 0.94 | exon | QRSL1 | U |
| 3609 | chr19 | 1648937 | 1648991 | Blood B cells | 0.02 | 0.93 | intron | TCF3 | U |
| 3610 | chr2 | 198009875 | 198009972 | Blood B cells | 0.04 | 0.94 | intron | ANKRD44 | U |
| 3611 | chr12 | 123305259 | 123305345 | Blood B cells | 0.02 | 0.91 | intron | CCDC62 | U |
| 3612 | chr21 | 346685413 | 34668701 | Blood B cells | 0.03 | 0.92 | intron, exon | IL10RB, IL10RB | U |
| 3613 | chr16 | 88079457 | 88079796 | Blood B cells | 0.02 | 0.91 | intron | BANP | U |
| 3614 | chr2 | 145319859 | 145320213 | Blood B cells | 0.03 | 0.92 | Intergenic | ZEB2 | U |
| 3615 | chr19 | 38540408 | 38540518 | Blood B cells | 0.05 | 0.93 | intron | SIPA1L3 | U |
| 3616 | chr12 | 132250981 | 132251257 | Blood B cells | 0.02 | 0.9 | intron | SFSWAP | U |
| 3617 | chr10 | 126289844 | 126290136 | Blood B cells | 0.03 | 0.91 | intron | LAPP | U |
| 3618 | chr19 | 2680895 | 2681205 | Blood B cells | 0.02 | 0.9 | intron | GNG7 | U |
| 3619 | chr6 | 99932478 | 99932972 | Blood B cells | 0.05 | 0.93 | intron | USP45 | U |
| 3620 | chr12 | 113488772 | 113488926 | Blood B cells | 0.03 | 0.9 | Intergenic | DTX1 | U |
| 3621 | chr1 | 185572197 | 185572415 | Blood B cells | 0.07 | 0.94 | Intergenic | HMCN1 | U |
| 3622 | chr1 | 161675214 | 161675517 | Blood B cells | 0.06 | 0.93 | Intergenic | FCRLA | U |
| 3623 | chr11 | 75535804 | 75535970 | Blood B cells | 0.03 | 0.89 | intron | UVRAG | U |
| 3624 | chr17 | 76736048 | 76736238 | Blood B cells | 0.07 | 0.93 | intron | CYTH1 | U |
| 3625 | chr8 | 28196033 | 28196315 | Blood B cells | 0.03 | 0.89 | intron | PNOC | U |
| 3626 | chr12 | 112452758 | 112453042 | Blood B cells | 0.08 | 0.94 | intron | ERP29 | U |
| 3627 | chr17 | 620029151 | 62003306 | Blood B cells | 0.02 | 0.88 | Intergenic, intergeni [symbol] | GH1, CD798 | U |
| 3628 | chr11 | 18251394 | 18251534 | Blood B cells | 0.02 | 0.87 | Intergenic | SAA4 | U |
| 3629 | chr21 | 44801137 | 44801320 | Blood B cells | 0.02 | 0.87 | Intergenic | SIK1 | U |
| 3630 | chr2 | 128335752 | 128335810 | Blood B cells | 0.1 | 0.94 | exon | MYO7B | U |
| 3631 | chr2 | 233989525 | 233989627 | Blood B cells | 0.04 | 0.88 | intron | INPP5D | U |
| 3632 | chr2 | 61691741 | 61691923 | Blood B cells | 0.05 | 0.89 | intron | USP34 | U |
| 3633 | chr16 | 88079879 | 88080175 | Blood B cells | 0.09 | 0.93 | intron | BANP | U |
| 3634 | chr6 | 74013762 | 74013930 | Blood B cells | 0.04 | 0.87 | intron | C6orf147 | U |
| 3635 | chr12 | 122447888 | 122448058 | Blood B cells | 0.05 | 0.88 | Intergenic | BCL7A | U |
| 3636 | chr11 | 35293206 | 35293377 | Blood B cells | 0.11 | 0.94 | intron | SLC1A2 | U |
| 3637 | chr9 | 136993737 | 136994083 | Blood B cells | 0.06 | 0.89 | Intergenic | WDR5 | U |
| 3638 | chr8 | 19467459 | 19467866 | Blood B cells | 0.08 | 0.91 | intron | CSGALNACT1 | U |
| 3639 | chr3 | 66436552 | 66437002 | Blood B cells | 0.07 | 0.9 | intron | LRIG1 | U |
| 3640 | chr16 | 87735396 | 87735517 | Blood B cells | 0.01 | 0.83 | Intergenic | KLHDC4 | U |
| 3641 | chr16 | 15101232 | 15101388 | Blood B cells | 0.11 | 0.93 | intron | PDXDC1 | U |
| 3642 | chr3 | 164754053 | 16475588 | Blood B cells | 0.1 | 0.92 | exon | RFTN1 | U |
| 3643 | chr16 | 68104804 | 68105016 | Blood B cells | 0.09 | 0.91 | intron, exon | DUS2L, DUS2L | U |
| 3644 | chr1 | 178574833 | 178575054 | Blood B cells | 0.11 | 0.93 | Intergenic | C1orf220 | U |
| 3645 | chr3 | 20168036 | 20168338 | Blood B cells | 0.12 | 0.94 | intron | KAT2B | U |
| 3646 | chr6 | 132005035 | 132005181 | Blood B cells | 0.13 | 0.91 | intron | ENPP3 | U |
| 3647 | chr6 | 43352107 | 43352270 | Blood B cells | 0.09 | 0.9 | Intergenic | ZNF318 | U |
| 3648 | chr7 | 403585 | 403752 | Blood B cells | 0.08 | 0.89 | Intergenic | LOC442497 | U |
| 3649 | chr13 | 100082003 | 100082172 | Blood B cells | 0.09 | 0.9 | Intergenic | MIR54BAN | U |
| 3650 | chr17 | 37706849 | 37707048 | Blood B cells | 0.09 | 0.9 | Intergenic | NEUROD2 | U |
| 3651 | chr3 | 13127967 | 13128167 | Blood B cells | 0.07 | 0.88 | Intergenic | IQSEC1 | U |
| 3652 | chr2 | 3258229 | 3258479 | Blood B cells | 0.11 | 0.92 | intron | TSSC1 | U |
| 3653 | chr10 | 1618865 | 1619186 | Blood B cells | 0.07 | 0.88 | intron | ADARB2 | U |
| 3654 | chr12 | 56769221 | 56769576 | Blood B cells | 0.07 | 0.88 | Intergenic | APOF | U |
| 3655 | chr8 | 28195582 | 28195952 | Blood B cells | 0.05 | 0.86 | intron | PNOC | U |
| 3656 | chr5 | 157986713 | 157987095 | Blood B cells | 0.09 | 0.9 | Intergenic | EBF1 | U |
| 3657 | chr4 | 15786449 | 15786843 | Blood B cells | 0.08 | 0.89 | intron | CD38 | U |
| 3658 | chr15 | 85999841 | 86000245 | Blood B cells | 0.12 | 0.93 | intron | AKAP13 | U |
| 3659 | chr11 | 70283519 | 70284008 | Blood B cells | 0.14 | 0.95 | Intergenic | CTTN | U |
| 3660 | chr8 | 11351835 | 11351929 | Blood B cells | 0.02 | 0.82 | exon | BLK | U |
| 3661 | chr17 | 3493609 | 3493903 | Blood B cells | 0.12 | 0.92 | exon | TRPV1 | U |
| 3662 | chr19 | 11548895 | 11549203 | Blood B cells | 0.11 | 0.91 | intron | PRKCSH | U |
| 3663 | chr21 | 46419673 | 46420022 | Blood B cells | 0.11 | 0.91 | intron | LINC00162 | U |
| 3664 | chr20 | 49427147 | 49427638 | Blood B cells | 0.12 | 0.92 | intron | BCAS4 | U |
| 3665 | chr15 | 100055770 | 100055858 | Blood B cells | 0.11 | 0.9 | Intergenic | MEF2A | U |
| 3666 | chr16 | 1530823 | 1530937 | Blood B cells | 0.04 | 0.83 | Intergenic | CLCN7 | U |
| 3667 | chr14 | 106285075 | 106285224 | Blood B cells | 0.06 | 0.85 | Intergenic | KIAA0125 | U |
| 3668 | chr5 | 132439040 | 132439295 | Blood B cells | 0.12 | 0.91 | intron | HSPA4 | U |
| 3669 | chr9 | 136826772 | 136827084 | Blood B cells | 0.11 | 0.9 | intron | VAV2 | U |
| 3670 | chr20 | 62702729 | 62702820 | Blood B cells | 0.06 | 0.84 | intron | TCEA2 | U |
| 3671 | chr2 | 89305601 | 89305840 | Blood B cells | 0.05 | 0.83 | Intergenic | MIR4436A | U |
| 3672 | chr1 | 25911759 | 25912018 | Blood B cells | 0.12 | 0.9 | Intergenic | MAN1C1 | U |
| 3673 | chr3 | 169564165 | 169564565 | Blood B cells | 0.12 | 0.9 | intron | LRRC31 | U |
| 3674 | chr1 | 181101405 | 181101828 | Blood B cells | 0.15 | 0.93 | Intergenic | IER5 | U |
| 3675 | chr19 | 35814453 | 35814908 | Blood B cells | 0.1 | 0.88 | Intergenic | CD22 | U |
| 3676 | chr6 | 138241334 | 138241799 | Blood B cells | 0.13 | 0.91 | Intergenic | LOC100130476 | U |
| 3677 | chr2 | 45922768 | 45922905 | Blood B cells | 0.12 | 0.89 | intron | PRKCE | U |
| 3678 | chr9 | 645656 | 645812 | Blood B cells | 0.15 | 0.92 | intron | KANK1 | U |
| 3679 | chr18 | 361310 | 361595 | Blood B cells | 0.13 | 0.9 | intron | COLEC12 | U |
| 3680 | chr12 | 122443613 | 122443967 | Blood B cells | 0.16 | 0.93 | Intergenic | BCL7A | U |
| 3681 | chr15 | 44886044 | 44886157 | Blood B cells | 0.12 | 0.88 | intron | SPG11 | U |
| 3682 | chr9 | 136993014 | 136993141 | Blood B cells | 0.17 | 0.93 | Intergenic | WDR5 | U |
| 3683 | chr2 | 89159064 | 89159274 | Blood B cells | 0.18 | 0.94 | Intergenic | MIR4436A | U |
| 3684 | chr4 | 1014995 | 1015467 | Blood B cells | 0.13 | 0.89 | intron | FGFRL1 | U |
| 3685 | chr14 | 103383229 | 103383582 | Blood B cells | 0.12 | 0.87 | Intergenic | AMN | U |
| 3686 | chr13 | 99090437 | 99090850 | Blood B cells | 0.16 | 0.91 | intron | FARP1 | U |
| 3687 | chr1 | 3496959 | 3497162 | Blood B cells | 0.08 | 0.82 | intron | MEGF6 | U |
| 3688 | chr1 | 101713983 | 101714195 | Blood B cells | 0.19 | 0.93 | Intergenic | S1PR1 | U |
| 3689 | chr11 | 36595278 | 36595594 | Blood B cells | 0.15 | 0.89 | exon | RAG1 | U |
| 3690 | chr11 | 66565836 | 66566020 | Blood B cells | 0.19 | 0.92 | intron | C11orf80 | U |
| 3691 | chr4 | 42333872 | 42333988 | Blood B cells | 0.1 | 0.82 | Intergenic | SHISA3 | U |
| 3692 | chr19 | 14688402 | 14688624 | Blood B cells | 0.15 | 0.87 | Intergenic | CLEC17A | U |
| 3693 | chr2 | 3257930 | 3258157 | Blood B cells | 0.23 | 0.95 | intron | TSSC1 | U |
| 3694 | chr15 | 28510556 | 28510729 | Blood B cells | 0.19 | 0.9 | intron | HERC2 | U |
| 3695 | chr10 | 80953658 | 80953873 | Blood B cells | 0.18 | 0.87 | intron | ZMIZ1 | U |
| 3696 | chr8 | 123929295 | 123929516 | Blood B cells | 0.23 | 0.92 | intron | ZHX2 | U |
| 3697 | chr5 | 130983529 | 130983911 | Blood B cells | 0.27 | 0.95 | intron | FNIP1 | U |
| 3698 | chr2 | 109810720 | 109810959 | Blood B cells | 0.26 | 0.92 | intron | SH3RF3 | U |
| 3699 | chr17 | 55081324 | 55081620 | Blood B cells | 0.32 | 0.95 | intron | SCPEP1 | U |
| 3700 | chr12 | 132255459 | 132255921 | Blood B cells | 0.28 | 0.91 | intron | SFSWAP | U |
| 3701 | chr12 | 56772333 | 56772693 | Blood B cells | 0.29 | 0.9 | Intergenic | APOF | U |
| 3702 | chr13 | 43562634 | 43562854 | Blood B cells | 0.17 | 0.95 | intron | EPSTI1 | U |
| 3703 | chr20 | 16373028 | 16373293 | Blood B cells | 0.02 | 0.94 | intron | KIF16B | U |
| 3704 | chr6 | 14254953 | 14255245 | Blood B cells | 0.06 | 0.95 | Intergenic | CD83 | U |
| 3705 | chr22 | 42332019 | 42332166 | Blood B cells | 0.05 | 0.9 | Intergenic | TNFRSF13C | U |
| 3706 | chr22 | 42752683 | 42752806 | Blood B cells | 0.02 | 0.86 | Intergenic | NFAM1 | U |
| 3707 | chr22 | 39469570 | 39469715 | Blood B cells | 0.01 | 0.85 | Intergenic | APOBEC3G | U |
| 3708 | chr22 | 33964527 | 33964904 | Blood B cells | 0.1 | 0.92 | intron | LARGE | U |
| 3709 | chr9 | 37189397 | 37189677 | Blood B cells | 0.14 | 0.95 | intron | ZCCHC7 | U |
| 3710 | chr22 | 24095077 | 24095518 | Blood B cells | 0.12 | 0.93 | TTS, intron | VPREB3, VPREB3 | U |
| 3711 | chr22 | 41379242 | 41379502 | Blood B cells | 0.18 | 0.91 | Intergenic | RBX1 | U |
| 3712 | chr22 | 30775765 | 30776040 | Blood B cells | 0.18 | 0.87 | exon, intron | RNF215, RNF215 | U |
| 3713 | chr11 | 67071719 | 67071793 | Blood B cells | 0.72 | 0.07 | intron | SSH3 | M |
| 3714 | chr16 | 81602029 | 81602179 | Blood B cells | 0.85 | 0.21 | intron | CMIP | M |
| 3715 | chr19 | 801276 | 801570 | Blood B cells | 0.73 | 0.1 | intron | PTBP1 | M |
| 3716 | chr11 | 118781778 | 118782027 | Blood B cells | 0.77 | 0.15 | promoter-TSS | BCL9L | M |
| 3717 | chr5 | 158407204 | 158407551 | Blood B cells | 0.71 | 0.09 | intron | EBF1 | M |
| 3718 | chr5 | 125839477 | 125839766 | Blood B cells | 0.75 | 0.14 | Intergenic | GRAMD3 | M |
| 3719 | chr7 | 99868422 | 99868626 | Blood B cells | 0.64 | 0.1 | intron | GATS | M |
| 3720 | chr11 | 118781633 | 118781712 | Blood B cells | 0.63 | 0.1 | promoter-TSS, prom [symbol] | MIR4492, BCL9L | M |
| 3721 | chr12 | 131322949 | 131323047 | Blood B cells | 0.58 | 0.06 | intron | STX2 | M |
| 3722 | chr11 | 316098 | 316385 | Blood B cells | 0.66 | 0.14 | TTS | IFITM1 | M |
| 3723 | chr18 | 24327182 | 24327297 | Blood B cells | 0.69 | 0.18 | Intergenic | LOC728606 | M |
| 3724 | chr7 | 101447905 | 101448166 | Blood B cells | 0.64 | 0.13 | Intergenic | CUX1 | M |
| 3725 | chr12 | 24103577 | 24103842 | Blood B cells | 0.61 | 0.11 | exon | SOX5 | M |
| 3726 | chr9 | 134283110 | 134283407 | Blood B cells | 0.6 | 0.11 | Intergenic | PRRC28 | M |
| 3727 | chr5 | 14582816 | 14582955 | Blood B cells | 0.63 | 0.16 | intron | FAM105A | M |
| 3728 | chr7 | 1498024 | 1498370 | Blood B cells | 0.61 | 0.16 | intron | MICALL2 | M |
| 3729 | chr19 | 56037380 | 56037716 | Blood B cells | 0.59 | 0.15 | Intergenic | SBK2 | M |
| 3730 | chr7 | 36192083 | 36192192 | Blood B cells | 0.54 | 0.15 | promoter-TSS | EEPD1 | M |
| 3731 | chr7 | 1014482781 | 101448507 | Blood B cells | 0.54 | 0.15 | Intergenic | CUX1 | M |
| 3732 | chr15 | 70877381 | 70877455 | Blood B cells | 0.53 | 0.16 | Intergenic | UACA | M |
| 3733 | chr22 | 50312817 | 50312922 | Blood B cells | 0.54 | 0.19 | promoter-TSS, prom [symbol] | CRELD2, ALG12 | M |
| 3734 | chr3 | 4909705 | 4909829043 | Blood B cells | 0.71 | 0.11 | Intergenic | BHLHE40-AS1 | M |
| 3735 | chr9 | 36985835 | 36986025 | Blood B cells | 0.65 | 0.08 | intron | PAX5 | M |
| 3736 | chr19 | 45281302 | 45281513 | Blood B cells | 0.58 | 0.13 | promoter-TSS, exon | CBLC, CBLC | M |
| 3737 | chr20 | 36514658 | 36514688 | Blood B cells | 0.58 | 0.24 | Intergenic | VSTM2L | M |
| 3738 | chr5 | 1475254 | 1475346 | Blood Granulocytes | 0.02 | 0.95 | intron | LPCAT1 | U |
| 3739 | chr19 | 19295400 | 19295569 | Blood Granulocytes | 0.02 | 0.94 | Intron | MEF2BNB-MEF28 | U |
| 3740 | chr2 | 8926362 | 8926831 | Blood Granulocytes | 0.01 | 0.92 | intron | KIDINS220 | U |
| 3741 | chr12 | 57894060 | 57894296 | Blood Granulocytes | 0.05 | 0.95 | exon | MARS | U |
| 3742 | chr3 | 150286079 | 150286436 | Blood Granulocytes | 0.04 | 0.94 | intron | EIF2A | U |
| 3743 | chr2 | 9035989 | 9036451 | Blood Granulocytes | 0.05 | 0.95 | intron | MBOAT2 | U |
| 3744 | chr16 | 46661322 | 46661443 | Blood Granulocytes | 0.02 | 0.91 | Intergenic | SHCBP1 | U |
| 3745 | chr7 | 100028821 | 100028979 | Blood Granulocytes | 0.01 | 0.89 | exon | MEPCE | U |
| 3746 | chr16 | 15053620 | 15054096 | Blood Granulocytes | 0.02 | 0.9 | Intergenic | PDXDC1 | U |
| 3747 | chr12 | 133242506 | 133242771 | Blood Granulocytes | 0.07 | 0.94 | intron | POLE | U |
| 3748 | chr7 | 105274407 | 105274692 | Blood Granulocytes | 0.06 | 0.92 | intron | ATXN7L1 | U |
| 3749 | chr16 | 67755255 | 67755383 | Blood Granulocytes | 0 | 0.86 | Intergenic | GFOD2 | U |
| 3750 | chr3 | 38322197 | 38322420 | Blood Granulocytes | 0.09 | 0.94 | Intergenic | SLC22A13 | U |
| 3751 | chr10 | 1344004353 | 134400535 | Blood Granulocytes | 0.08 | 0.91 | intron | INPP5A | U |
| 3752 | chr12 | 76934829 | 76935130 | Blood Granulocytes | 0.1 | 0.93 | intron | OSBPL8 | U |
| 3753 | chr7 | 105082754 | 105082935 | Blood Granulocytes | 0.11 | 0.94 | Intergenic | SRPK2 | U |
| 3754 | chr11 | 113641630 | 113641842 | Blood Granulocytes | 0.13 | 0.94 | intron | ZW10 | U |
| 3755 | chr1 | 3563696 | 3563873 | Blood Granulocytes | 0.09 | 0.88 | intron | WRAP73 | U |
| 3756 | chr16 | 4678631 | 4678872 | Blood Granulocytes | 0.19 | 0.96 | Intron | MGRN1 | U |
| 3757 | chr12 | 133257773 | 133257855 | Blood Granulocytes | 0.2 | 0.96 | intron | POLE | U |
| 3758 | chr12 | 67058221 | 6705998 | Blood Granulocytes | 0.17 | 0.93 | intron | CHD4 | U |
| 3759 | chr7 | 65214762 | 65214844 | Blood Granulocytes | 0.02 | 0.95 | intron | LOC441242 | U |
| 3760 | chr12 | 113770034 | 113770170 | Blood Granulocytes | 0.01 | 0.94 | intron | SLC24A6 | U |
| 3761 | chr10 | 3815120 | 3815328 | Blood Granulocytes | 0.01 | 0.94 | Intergenic | KLF6 | U |
| 3762 | chrX | 44780151 | 44780402 | Blood Granulocytes | 0.02 | 0.95 | intron | KDM6A | U |
| 3763 | chr15 | 50266301 | 50266366 | Blood Granulocytes | 0.02 | 0.94 | intron | ATP884 | U |
| 3764 | chr2 | 30935561 | 30935725 | Blood Granulocytes | 0.01 | 0.93 | Intergenic | CAPN13 | U |
| 3765 | chr15 | 63180784 | 63180872 | Blood Granulocytes | 0.01 | 0.92 | Intergenic | MIR190A | U |
| 3766 | chr20 | 61582887 | 61583050 | Blood Granulocytes | 0.01 | 0.92 | Intergenic, promote [symbol] | SLC17A9, SLC17A9 | U |
| 3767 | chr8 | 131000351 | 131000563 | Blood Granulocytes | 0.02 | 0.93 | intron | FAM498 | U |
| 3768 | chr1 | 10211350 | 10211689 | Blood Granulocytes | 0.01 | 0.92 | exon | UBE4B | U |
| 3769 | chr2 | 64234846 | 64235273 | Blood Granulocytes | 0.02 | 0.93 | intron | VPS54 | U |
| 3770 | chr1 | 6035727 | 6035846 | Blood Granulocytes | 0.01 | 0.91 | intron | NPHP4 | U |
| 3771 | chr19 | 4464265 | 4464433 | Blood Granulocytes | 0.01 | 0.91 | Intergenic | UBXN6 | U |
| 3772 | chr4 | 140852379 | 140852580 | Blood Granulocytes | 0.02 | 0.92 | intron | MAML3 | U |
| 3773 | chr16 | 89365093 | 89365308 | Blood Granulocytes | 0.01 | 0.91 | intron | ANKRD11 | U |
| 3774 | chr2 | 98429359 | 98429682 | Blood Granulocytes | 0.01 | 0.91 | intron | TMEM131 | U |
| 3775 | chr2 | 64642834 | 64643180 | Blood Granulocytes | 0.02 | 0.92 | Intergenic | GALSL | U |
| 3776 | chr10 | 6374510 | 6374605 | Blood Granulocytes | 0.04 | 0.93 | intron | LOC399715 | U |
| 3777 | chr11 | 47734458 | 47734630 | Blood Granulocytes | 0.05 | 0.94 | intron | AGBL2 | U |
| 3778 | chr14 | 90440873 | 90441060 | Blood Granulocytes | 0.01 | 0.9 | intron | TDP1 | U |
| 3779 | chr17 | 2203353 | 2203570 | Blood Granulocytes | 0.02 | 0.91 | exon | SMG6 | U |
| 3780 | chr2 | 99760705 | 99761005 | Blood Granulocytes | 0.02 | 0.91 | intron | C2orf15 | U |
| 3781 | chr3 | 183662759 | 183663081 | Blood Granulocytes | 0.01 | 0.9 | intron | ABCC5 | U |
| 3782 | chr3 | 128710307 | 128710637 | Blood Granulocytes | 0.03 | 0.92 | intron | KIAA1257 | U |
| 3783 | chr1 | 1097120811 | 109712163 | Blood Granulocytes | 0.03 | 0.91 | intron | KIAA1324 | U |
| 3784 | chr2 | 30666895 | 30667038 | Blood Granulocytes | 0.03 | 0.91 | Intergenic | LCLAT1 | U |
| 3785 | chr15 | 78291531 | 78291693 | Blood Granulocytes | 0.01 | 0.89 | intron | TBC1D2B | U |
| 3786 | chr8 | 61496608 | 61496839 | Blood Granulocytes | 0.03 | 0.91 | intron | RAB2A | U |
| 3787 | chr3 | 5131788 | 5131877 | Blood Granulocytes | 0.01 | 0.88 | Intergenic | ARL8B | U |
| 3788 | chr16 | 895669413 | 89567064 | Blood Granulocytes | 0.01 | 0.88 | Intergenic | SPG7 | U |
| 3789 | chr9 | 1398065561 | 139806684 | Blood Granulocytes | 0.03 | 0.9 | intron | TRAF2 | U |
| 3790 | chr17 | 64421921 | 64422066 | Blood Granulocytes | 0.01 | 0.88 | intron | PRKCA | U |
| 3791 | chr2 | 9136659 | 9136856 | Blood Granulocytes | 0.06 | 0.93 | intron | MBOAT2 | U |
| 3792 | chr19 | 41238916 | 41239115 | Blood Granulocytes | 0.02 | 0.89 | intron | ITPKC | U |
| 3793 | chr15 | 70096332 | 70096600 | Blood Granulocytes | 0.07 | 0.94 | Intergenic | LINC00593 | U |
| 3794 | chr7 | 23327214 | 23327556 | Blood Granulocytes | 0.01 | 0.88 | Intergenic | MALSU1 | U |
| 3795 | chr4 | 146708334 | 146708388 | Blood Granulocytes | 0.09 | 0.95 | intron | ZNF827 | U |
| 3796 | chr15 | 99074273 | 99074398 | Blood Granulocytes | 0.05 | 0.91 | Intergenic | FAM169B | U |
| 3797 | chr12 | 1243416271 | 124341771 | Blood Granulocytes | 0.07 | 0.93 | exon | DNAH10 | U |
| 3798 | chr15 | 65585965 | 65586123 | Blood Granulocytes | 0.01 | 0.87 | Intergenic | PARP16 | U |
| 3799 | chr9 | 139812189 | 139812430 | Blood Granulocytes | 0.07 | 0.93 | intron | TRAF2 | U |
| 3800 | chr17 | 40534781 | 4053742 | Blood Granulocytes | 0.04 | 0.9 | intron | CYB5D2 | U |
| 3801 | chr17 | 33319549 | 33319932 | Blood Granulocytes | 0.06 | 0.92 | intron | LIG3 | U |
| 3802 | chr1 | 213066546 | 213066948 | Blood Granulocytes | 0.09 | 0.95 | intron | FLVCR1 | U |
| 3803 | chr19 | 2303624 | 2303682 | Blood Granulocytes | 0.07 | 0.92 | intron | LINGO3 | U |
| 3804 | chr7 | 99745687 | 99745752 | Blood Granulocytes | 0.07 | 0.92 | promoter-TSS | LAMTOR4 | U |
| 3805 | chr16 | 3663807 | 3663900 | Blood Granulocytes | 0.11 | 0.96 | Intergenic | SLX4 | U |
| 3806 | chr18 | 9704835 | 9705006 | Blood Granulocytes | 0.01 | 0.86 | Intergenic | RAB31 | U |
| 3807 | chr13 | 21045259 | 21045318 | Blood Granulocytes | 0.09 | 0.93 | intron | CRYL1 | U |
| 3808 | chr17 | 4081029 | 4081148 | Blood Granulocytes | 0.09 | 0.93 | intron | ANKFY1 | U |
| 3809 | chr7 | 2185546 | 2185719 | Blood Granulocytes | 0.1 | 0.94 | intron | MAD1L1 | U |
| 3810 | chr1 | 21786129 | 21786323 | Blood Granulocytes | 0.11 | 0.95 | intron | NBPF3 | U |
| 3811 | chr11 | 33720840 | 33721148 | Blood Granulocytes | 0.03 | 0.87 | intron | C11orf91 | U |
| 3812 | chr15 | 91488251 | 91488695 | Blood Granulocytes | 0.08 | 0.92 | intron | UNC45A | U |
| 3813 | chr9 | 132286031 | 132286384 | Blood Granulocytes | 0.1 | 0.93 | Intergenic | LOC100506190 | U |
| 3814 | chr5 | 78498499 | 78498589 | Blood Granulocytes | 0.11 | 0.93 | Intergenic | JMY | U |
| 3815 | chr11 | 3953224 | 3953341 | Blood Granulocytes | 0.12 | 0.94 | intron | STIM1 | U |
| 3816 | chr10 | 105092700 | 105092833 | Blood Granulocytes | 0.11 | 0.93 | intron | PCGFS | U |
| 3817 | chr10 | 70826761 | 70826942 | Blood Granulocytes | 0.12 | 0.94 | Intergenic | SRGN | U |
| 3818 | chr17 | 47810066 | 47810180 | Blood Granulocytes | 0.1 | 0.91 | intron | FAM117A | U |
| 3819 | chr9 | 126185525 | 126185683 | Blood Granulocytes | 0.11 | 0.92 | intron | DENND1A | U |
| 3820 | chr1 | 172769019 | 172769212 | Blood Granulocytes | 0.11 | 0.92 | Intergenic | FASLG | U |
| 3821 | chr1 | 2027115801 | 202711865 | Blood Granulocytes | 0.14 | 0.95 | exon | KDMSB | U |
| 3822 | chr10 | 69962050 | 69962185 | Blood Granulocytes | 0.07 | 0.87 | intron | MYPN | U |
| 3823 | chr2 | 95779443 | 95779605 | Blood Granulocytes | 0.14 | 0.94 | intron | MRPS5 | U |
| 3824 | chr5 | 145560642 | 145560809 | Blood Granulocytes | 0.15 | 0.95 | intron | LARS | U |
| 3825 | chr18 | 12025394 | 12025602 | Blood Granulocytes | 0.13 | 0.93 | Intron | IMPA2 | U |
| 3826 | chr19 | 1423535 | 1423830 | Blood Granulocytes | 0.13 | 0.93 | intron | DAZAP1 | U |
| 3827 | chr12 | 132358250 | 132358563 | Blood Granulocytes | 0.1 | 0.9 | Intergenic | ULK1 | U |
| 3828 | chr19 | 19292201 | 19292561 | Blood Granulocytes | 0.11 | 0.91 | TTS | MEF2BNB | U |
| 3829 | chr9 | 130511864 | 130511940 | Blood Granulocytes | 0.11 | 0.9 | exon | SH2D3C | U |
| 3830 | chr10 | 323625 | 323769 | Blood Granulocytes | 0.12 | 0.91 | intron | DIP2C | U |
| 3831 | chr16 | 69047468 | 69047685 | Blood Granulocytes | 0.15 | 0.93 | intron | TANGO6 | U |
| 3832 | chr7 | 2874556 | 2874621 | Blood Granulocytes | 0.15 | 0.92 | intron | GNA12 | U |
| 3833 | chr12 | 990207271 | 99020999 | Blood Granulocytes | 0.16 | 0.93 | intron | IKBIP | U |
| 3834 | chr1 | 6100977 | 6101098 | Blood Granulocytes | 0.11 | 0.87 | intron | KCNAB2 | U |
| 3835 | chr17 | 47312993 | 47313289 | Blood Granulocytes | 0.01 | 0.77 | Intergenic | PHOSPHO1 | U |
| 3836 | chr10 | 120801913 | 120802124 | Blood Granulocytes | 0.19 | 0.92 | exon | EIF3A | U |
| 3837 | chr16 | 75632204 | 75632503 | Blood Granulocytes | 0.21 | 0.94 | exon, Intergenic | ADAT1, ADAT1 | U |
| 3838 | chr18 | 77489211 | 77489347 | Blood Granulocytes | 0.24 | 0.96 | intron | CTDP1 | U |
| 3839 | chr11 | 63919718 | 63919836 | Blood Granulocytes | 0.2 | 0.91 | exon, intron | MACROD1, MACROD [symbol] | U |
| 3840 | chr9 | 124224459 | 124224697 | Blood Granulocytes | 0.19 | 0.9 | Intron | GGTA1P | U |
| 3841 | chr12 | 11937527 | 11937785 | Blood Granulocytes | 0.22 | 0.93 | Intron | ETV6 | U |
| 3842 | chr11 | 68529887 | 68530203 | Blood Granulocytes | 0.22 | 0.93 | intron | CPT1A | U |
| 3843 | chr16 | 4686197 | 4686426 | Blood Granulocytes | 0.2 | 0.9 | intron | MGRN1 | U |
| 3844 | chr11 | 73553706 | 73554049 | Blood Granulocytes | 0.23 | 0.93 | intron | MRPL48 | U |
| 3845 | chr8 | 74870779 | 74871205 | Blood Granulocytes | 0.23 | 0.92 | intron | TCEB1 | U |
| 3846 | chr8 | 28203960 | 28204405 | Blood Granulocytes | 0.19 | 0.88 | exon | ZNF395 | U |
| 3847 | chr2 | 234166507 | 234166753 | Blood Granulocytes | 0.28 | 0.96 | intron | ATG16L1 | U |
| 3848 | chr8 | 121553241 | 121553667 | Blood Granulocytes | 0.25 | 0.93 | intron | SNT81 | U |
| 3849 | chr12 | 6790100 | 6790430 | Blood Granulocytes | 0.26 | 0.93 | intron | ZNF384 | U |
| 3850 | chr14 | 102676909 | 102677377 | Blood Granulocytes | 0.04 | 0.91 | TTS | WDR20 | U |
| 3851 | chr8 | 68262826 | 68263288 | Blood Granulocytes | 0.04 | 0.9 | Intergenic | ARFGEF1 | U |
| 3852 | chr20 | 48270862 | 48271230 | Blood Granulocytes | 0.01 | 0.93 | intron | B4GALTS | U |
| 3853 | chr21 | 46335363 | 46335431 | Blood Granulocytes | 0 | 0.89 | intron | ITGB2 | U |
| 3854 | chr20 | 33197229 | 33197638 | Blood Granulocytes | 0.11 | 0.95 | intron | PIGU | U |
| 3855 | chr7 | 8046949 | 8047052 | Blood Granulocytes | 0.23 | 0.96 | intron | GLCCI1 | U |
| 3856 | chr9 | 101592971 | 101593231 | Blood Granulocytes | 0.02 | 0.9 | intron | GALNT12 | U |
| 3857 | chr20 | 47779597 | 47779715 | Blood Granulocytes | 0.06 | 0.93 | intron | STAU1 | U |
| 3858 | chr9 | 92271825 | 92271989 | Blood Granulocytes | 0.03 | 0.9 | intron | UNQ6494 | U |
| 3859 | chr9 | 84254232 | 84254591 | Blood Granulocytes | 0.06 | 0.93 | intron | TLE1 | U |
| 3860 | chr9 | 371314401 | 37131855 | Blood Granulocytes | 0.09 | 0.92 | intron | ZCCHC7 | U |
| 3861 | chr9 | 88899143 | 88899283 | Blood Granulocytes | 0.1 | 0.92 | Intergenic | ISCA1 | U |
| 3862 | chr6 | 14827822 | 14827984 | Blood Granulocytes | 0.12 | 0.93 | Intergenic | JARID2 | U |
| 3863 | chr19 | 17185295 | 17185540 | Blood Granulocytes | 0.66 | 0.12 | intron, promoter-TS [symbol] | HAUS8, HAUS8 | M |
| 3864 | chr1 | 25253237 | 25253456 | Blood Granulocytes | 0.67 | 0.14 | intron | RUNX3 | M |
| 3865 | chr12 | 117096360 | 117096543 | Blood Granulocytes | 0.57 | 0.06 | Intergenic | C12orf49 | M |
| 3866 | chr18 | 44701530 | 44701801 | Blood Granulocytes | 0.65 | 0.14 | intron | IER3IP1 | M |
| 3867 | chr10 | 15209430 | 15209669 | Blood Granulocytes | 0.62 | 0.12 | intron | NMT2 | M |
| 3868 | chr17 | 80339122 | 80339255 | Blood Granulocytes | 0.65 | 0.17 | Intergenic | UTS2R | M |
| 3869 | chr19 | 2503973 | 2503999 | Blood Granulocytes | 0.69 | 0.22 | Intergenic | GADD45B | M |
| 3870 | chr13 | 108866962 | 108867198 | Blood Granulocytes | 0.61 | 0.14 | promoter-TSS | LIG4 | M |
| 3871 | chr19 | 39339082 | 39339288 | Blood Granulocytes | 0.62 | 0.16 | intron | HNRNPL | M |
| 3872 | chr6 | 38670144 | 38670313 | Blood Granulocytes | 0.61 | 0.16 | intron | GLO1 | M |
| 3873 | chr21 | 35446809 | 35447021 | Blood Granulocytes | 0.54 | 0.09 | intron | MRPS6 | M |
| 3874 | chr16 | 21059351 | 21059646 | Blood Granulocytes | 0.62 | 0.17 | intron | DNAH3 | M |
| 3875 | chr11 | 72392383 | 72392660 | Blood Granulocytes | 0.57 | 0.13 | Intergenic | PDE2A | M |
| 3876 | chr8 | 55013175 | 55013407 | Blood Granulocytes | 0.61 | 0.18 | intron | LYPLA1 | M |
| 3877 | chr20 | 2450253 | 2450335 | Blood Granulocytes | 0.54 | 0.14 | intron | SNRPB | M |
| 3878 | chr4 | 6718724 | 6718935 | Blood Granulocytes | 0.54 | 0.14 | exon | BLOC154 | M |
| 3879 | chr1 | 247093815 | 247094007 | Blood Granulocytes | 0.61 | 0.23 | intron | AHCTF1 | M |
| 3880 | chr22 | 46938036 | 46938104 | Blood Granulocytes | 0.6 | 0.24 | Intergenic | CELSRI | M |
| 3881 | chr18 | 60985262 | 60985381 | Blood Granulocytes | 0.54 | 0.18 | TTS, exon | BCL2, BCL2 | M |
| 3882 | chr5 | 180611773 | 180611842 | Blood Granulocytes | 0.64 | 0.29 | Intergenic | TRIM7 | M |
| 3883 | chr19 | 32329465 | 32329599 | Blood Granulocytes | 0.55 | 0.2 | Intergenic | THEGS | M |
| 3884 | chr19 | 5877489 | 55877514 | Blood Granulocytes | 0.65 | 0.35 | exon | IL11 | M |
| 3885 | chr7 | 30678941 | 3068044 | Blood Granulocytes | 0.67 | 0.15 | intron | CARD11 | M |
| 3886 | chr22 | 39919480 | 39919725 | Blood Granulocytes | 0.57 | 0.11 | TTS | ATFA | M |
| 3887 | chr11 | 73733661 | 73733929 | Blood Monocytes + Macrophages | 0.12 | 0.94 | Intergenic | UCP3 | U |
| 3888 | chr1 | 60046062 | 60046409 | Blood Monocytes + Macrophages | 0.11 | 0.92 | intron | FGGY | U |
| 3889 | chr16 | 29184910 | 29185241 | Blood Monocytes + Macrophages | 0.12 | 0.92 | Intergenic | RRN3P2 | U |
| 3890 | chr11 | 33219522 | 33219704 | Blood Monocytes + Macrophages | 0.13 | 0.92 | Intergenic | CSTF3-AS1 | U |
| 3891 | chr2 | 234151559 | 234151741 | Blood Monocytes + Macrophages | 0.15 | 0.94 | Intergenic | ATG16L1 | U |
| 3892 | chr7 | 106668694 | 106668906 | Blood Monocytes + Macrophages | 0.16 | 0.94 | Intergenic | PRKAR2B | U |
| 3893 | chr2 | 64981299 | 64981412 | Blood Monocytes + Macrophages | 0.16 | 0.93 | Intergenic | SERTAD2 | U |
| 3894 | chr12 | 25120044 | 25120202 | Blood Monocytes + Macrophages | 0.16 | 0.92 | Intergenic | BCAT1 | U |
| 3895 | chr2 | 122044859 | 122045164 | Blood Monocytes + Macrophages | 0.11 | 0.87 | Intergenic | TFCP2L1 | U |
| 3896 | chr21 | 478520401 | 47852138 | Blood Monocytes + Macrophages | 0.16 | 0.91 | exon | PCNT | U |
| 3897 | chr19 | 523147191 | 52314907 | Blood Monocytes + Macrophages | 0.14 | 0.89 | intron | FPR3 | U |
| 3898 | chr12 | 10328181 | 1033044 | Blood Monocytes + Macrophages | 0.2 | 0.93 | intron | RAD52 | U |
| 3899 | chr14 | 1036572701 | 103657436 | Blood Monocytes + Macrophages | 0.19 | 0.91 | Intergenic | LINC00605 | U |
| 3900 | chr19 | 52327634 | 52327730 | Blood Monocytes + Macrophages | 0.21 | 0.91 | exor | FPR3 | U |
| 3901 | chr12 | 14405611 | 14405730 | Blood Monocytes + Macrophages | 0.23 | 0.93 | Intergenic | ATF7IP | U |
| 3902 | chr14 | 103909521 | 103909986 | Blood Monocytes + Macrophages | 0.24 | 0.93 | intron | MARK3 | U |
| 3903 | chr3 | 13620469 | 13620615 | Blood Monocytes + Macrophages | 0.18 | 0.87 | intron | FBLN2 | U |
| 3904 | chr10 | 134264798 | 134264878 | Blood Monocytes + Macrophages | 0.25 | 0.93 | Intergenic | C10orf91 | U |
| 3905 | chr2 | 152905911 | 152905994 | Blood Monocytes + Macrophages | 0.24 | 0.9 | intron | CACNB4 | U |
| 3906 | chr19 | 192387243 | 19239022 | Blood Monocytes + Macrophages | 0.28 | 0.94 | intron | TMEM161A | U |
| 3907 | chr7 | 2284482 | 2284611 | Blood Monocytes + Macrophages | 0.3 | 0.94 | intron | NUDT1 | U |
| 3908 | chr10 | 114911613 | 114911856 | Blood Monocytes + Macrophages | 0.36 | 0.94 | intron | TCF7L2 | U |
| 3909 | chr1 | 46483872 | 46484334 | Blood Monocytes + Macrophages | 0.36 | 0.93 | intron | MAST2 | U |
| 3910 | chr19 | 10236782 | 10236959 | Blood Monocytes + Macrophages | 0.1 | 0.92 | Intergenic | EIF3G | U |
| 3911 | chr12 | 112326273 | 112326443 | Blood Monocytes + Macrophages | 0.14 | 0.94 | exon | MAPKAPK5 | U |
| 3912 | chr2 | 102138126 | 102138223 | Blood Monocytes + Macrophages | 0.13 | 0.92 | Intergenic | RFX8 | U |
| 3913 | chr6 | 134696479 | 134696724 | Blood Monocytes + Macrophages | 0.15 | 0.94 | Intergenic | LOC154092 | U |
| 3914 | chr17 | 64434250 | 64434490 | Blood Monocytes + Macrophages | 0.09 | 0.87 | intron | PRKCA | U |
| 3915 | chr8 | 27248671 | 27248761 | Blood Monocytes + Macrophages | 0.15 | 0.91 | intron | PTK2B | U |
| 3916 | chr3 | 196302048 | 196302102 | Blood Monocytes + Macrophages | 0.14 | 0.89 | intron | FBXO45 | U |
| 3917 | chr7 | 140194671 | 140194744 | Blood Monocytes + Macrophages | 0.19 | 0.94 | Intergenic | MKRN1 | U |
| 3918 | chr17 | 79300342 | 79300419 | Blood Monocytes + Macrophages | 0.14 | 0.89 | intron | TMEM105 | U |
| 3919 | chr20 | 52313293 | 52313611 | Blood Monocytes + Macrophages | 0.17 | 0.92 | Intergenic | ZNF217 | U |
| 3920 | chr17 | 78186771 | 78186866 | Blood Monocytes + Macrophages | 0.17 | 0.91 | intron | SGSH | U |
| 3921 | chr8 | 28961290 | 28961451 | Blood Monocytes + Macrophages | 0.14 | 0.88 | intron | KIF13B | U |
| 3922 | chr2 | 219564598 | 219564772 | Blood Monocytes + Macrophages | 0.18 | 0.92 | intron | STK36 | U |
| 3923 | chr1 | 40499816 | 40499992 | Blood Monocytes + Macrophages | 0.11 | 0.85 | Intergenic | CAP1 | U |
| 3924 | chr19 | 46396750 | 46397031 | Blood Monocytes + Macrophages | 0.12 | 0.86 | intron | MYPOP | U |
| 3925 | chr12 | 1032945 | 1033045 | Blood Monocytes + Macrophages | 0.22 | 0.95 | intron | RAD52 | U |
| 3926 | chr1 | 184886535 | 184886642 | Blood Monocytes + Macrophages | 0.16 | 0.89 | intron | FAM129A | U |
| 3927 | chr2 | 100402240 | 100402431 | Blood Monocytes + Macrophages | 0.11 | 0.84 | intron | AFF3 | U |
| 3928 | chr1 | 6116351 | 6116401 | Blood Monocytes + Macrophages | 0.17 | 0.89 | intron | KCNAB2 | U |
| 3929 | chr1 | 179919826 | 179919985 | Blood Monocytes + Macrophages | 0.2 | 0.92 | Intergenic | CEP350 | U |
| 3930 | chr16 | 16035816 | 16036072 | Blood Monocytes + Macrophages | 0.17 | 0.89 | Intergenic | ABCC1 | U |
| 3931 | chrX | 44305596 | 44305693 | Blood Monocytes + Macrophages | 0.14 | 0.85 | Intergenic | FUNDC1 | U |
| 3932 | chr19 | 54349022 | 54349161 | Blood Monocytes + Macrophages | 0.17 | 0.88 | Intergenic | NLRP12 | U |
| 3933 | chr3 | 185367216 | 185367389 | Blood Monocytes + Macrophages | 0.16 | 0.87 | intron | IGF28P2 | U |
| 3934 | chr17 | 19826637 | 19826881 | Blood Monocytes + Macrophages | 0.24 | 0.95 | intron | AKAP10 | U |
| 3935 | chr12 | 124426212 | 124426498 | Blood Monocytes + Macrophages | 0.2 | 0.91 | intron | CCDC92 | U |
| 3936 | chr16 | 53838299 | 53838631 | Blood Monocytes + Macrophages | 0.23 | 0.94 | intron | FTO | U |
| 3937 | chr13 | 41355868 | 41356209 | Blood Monocytes + Macrophages | 0.18 | 0.89 | Intergenic | SLC25A15 | U |
| 3938 | chr3 | 143717052 | 143717459 | Blood Monocytes + Macrophages | 0.16 | 0.87 | Intergenic | C3orf58 | U |
| 3939 | chr20 | 52314857 | 52314996 | Blood Monocytes + Macrophages | 0.21 | 0.91 | Intergenic | ZNF217 | U |
| 3940 | chr1 | 117269817 | 117270000 | Blood Monocytes + Macrophages | 0.18 | 0.88 | Intergenic | CD2 | U |
| 3941 | chr21 | 47852152 | 47852335 | Blood Monocytes + Macrophages | 0.17 | 0.87 | intron | PCNT | U |
| 3942 | chr16 | 4038109 | 4038296 | Blood Monocytes + Macrophages | 0.18 | 0.88 | intron | ADCY9 | U |
| 3943 | chr5 | 142544963 | 142545178 | Blood Monocytes + Macrophages | 0.22 | 0.92 | Intron | ARHGAP26 | U |
| 3944 | chr12 | 117495495 | 117495754 | Blood Monocytes + Macrophages | 0.19 | 0.89 | intron | TESC | U |
| 3945 | chr17 | 25740818 | 25741249 | Blood Monocytes + Macrophages | 0.18 | 0.88 | Intergenic | TBC1D3P5 | U |
| 3946 | chr1 | 113425936 | 113426010 | Blood Monocytes + Macrophages | 0.18 | 0.87 | Intergenic | AKR7A2P1 | U |
| 3947 | chr19 | 6686504 | 6686578 | Blood Monocytes + Macrophages | 0.17 | 0.86 | intron, exon | C3, C3 | U |
| 3948 | chr19 | 7608727 | 7608825 | Blood Monocytes + Macrophages | 0.22 | 0.91 | intron | PNPLA6 | U |
| 3949 | chr15 | 72756712 | 72756899 | Blood Monocytes + Macrophages | 0.21 | 0.9 | Intergenic | ARIH1 | U |
| 3950 | chr12 | 133058610 | 133058836 | Blood Monocytes + Macrophages | 0.16 | 0.85 | Intergenic | FBRSLI | U |
| 3951 | chr4 | 1327181 | 1327450 | Blood Monocytes + Macrophages | 0.23 | 0.92 | intron | MAEA | U |
| 3952 | chr2 | 8417289 | 8417424 | Blood Monocytes + Macrophages | 0.22 | 0.9 | intron | LINC00299 | U |
| 3953 | chr12 | 111624255 | 111624399 | Blood Monocytes + Macrophages | 0.12 | 0.8 | intron | CUX2 | U |
| 3954 | chr10 | 134987822 | 134987974 | Blood Monocytes + Macrophages | 0.23 | 0.91 | intron | KNDC1 | U |
| 3955 | chr11 | 63401540 | 63401775 | Blood Monocytes + Macrophages | 0.24 | 0.92 | intron | ATL3 | U |
| 3956 | chr1 | 42157705 | 42157945 | Blood Monocytes + Macrophages | 0.24 | 0.92 | intron | HIVEP3 | U |
| 3957 | chr10 | 50493567 | 50493836 | Blood Monocytes + Macrophages | 0.12 | 0.8 | Intergenic | C10orf71 | U |
| 3958 | chr15 | 81011275 | 81011754 | Blood Monocytes + Macrophages | 0.24 | 0.92 | intron | FAM108C1 | U |
| 3959 | chr7 | 139692528 | 139692736 | Blood Monocytes + Macrophages | 0.22 | 0.89 | intron | TBXASI | U |
| 3960 | chr10 | 105225247 | 105225401 | Blood Monocytes + Macrophages | 0.21 | 0.87 | Intergenic | CALHM1 | U |
| 3961 | chr19 | 47328399 | 47328584 | Blood Monocytes + Macrophages | 0.19 | 0.85 | Intergenic | SNAR-E | U |
| 3962 | chr8 | 26290788 | 26290992 | Blood Monocytes + Macrophages | 0.21 | 0.87 | Intergenic | BNIP3L | U |
| 3963 | chr1 | 6116603 | 6116830 | Blood Monocytes + Macrophages | 0.2 | 0.86 | intron | KCNAB2 | U |
| 3964 | chr12 | 111129103 | 111129424 | Blood Monocytes + Macrophages | 0.23 | 0.89 | Intergenic | HVCN1 | U |
| 3965 | chr2 | 148400250 | 148400672 | Blood Monocytes + Macrophages | 0.23 | 0.88 | Intergenic | ACVR2A | U |
| 3966 | chr7 | 1955684 | 1955910 | Blood Monocytes + Macrophages | 0.26 | 0.9 | intron | MAD1L1 | U |
| 3967 | chr15 | 75447339 | 75447636 | Blood Monocytes + Macrophages | 0.27 | 0.91 | Intergenic | C15orf39 | U |
| 3968 | chr16 | 75605929 | 75606017 | Blood Monocytes + Macrophages | 0.28 | 0.91 | intron | GABARAPL2 | U |
| 3969 | chr4 | 1220762 | 1220877 | Blood Monocytes + Macrophages | 0.25 | 0.88 | intron | CTBP1 | U |
| 3970 | chr1 | 3759631 | 3760019 | Blood Monocytes + Macrophages | 0.3 | 0.93 | intron | CEP104 | U |
| 3971 | chr7 | 76615403 | 76615523 | Blood Monocytes + Macrophages | 0.25 | 0.87 | intron | DTX2P1-UPK3BP1-PM [symbol] | U |
| 3972 | chr19 | 47262540 | 47262759 | Blood Monocytes + Macrophages | 0.21 | 0.83 | TTS | FKRP | U |
| 3973 | chr4 | 103514429 | 103514692 | Blood Monocytes + Macrophages | 0.29 | 0.91 | intron | NFKB1 | U |
| 3974 | chr12 | 402268 | 402558 | Blood Monocytes + Macrophages | 0.32 | 0.94 | intron | KDM5A | U |
| 3975 | chr18 | 10797425 | 10797759 | Blood Monocytes + Macrophages | 0.22 | 0.84 | exon, intron | PIEZO2, PIEZO2 | U |
| 3976 | chr10 | 738511953 | 73851333 | Blood Monocytes + Macrophages | 0.32 | 0.93 | Intergenic | SPOCK2 | U |
| 3977 | chr1 | 26711952 | 26712115 | Blood Monocytes + Macrophages | 0.24 | 0.85 | Intergenic | ZNF683 | U |
| 3978 | chr9 | 140538629 | 140538902 | Blood Monocytes + Macrophages | 0.29 | 0.9 | intron | EHMT1 | U |
| 3979 | chr17 | 19514244 | 19514536 | Blood Monocytes + Macrophages | 0.25 | 0.86 | Intergenic | ALDH3A2 | U |
| 3980 | chr10 | 95146992 | 95147326 | Blood Monocytes + Macrophages | 0.29 | 0.9 | Intron | MYOF | U |
| 3981 | chr11 | 16812551 | 16812905 | Blood Monocytes + Macrophages | 0.28 | 0.89 | exon | PLEKHA7 | U |
| 3982 | chr1 | 117040473 | 117040700 | Blood Monocytes + Macrophages | 0.25 | 0.85 | Intergenic | CD58 | U |
| 3983 | chr6 | 149591904 | 149592153 | Blood Monocytes + Macrophages | 0.32 | 0.92 | Intergenic | TAB2 | U |
| 3984 | chr17 | 45149888 | 45150225 | Blood Monocytes + Macrophages | 0.27 | 0.87 | Intergenic | RPRML | U |
| 3985 | chr18 | 60244241 | 60244632 | Blood Monocytes + Macrophages | 0.3 | 0.9 | exon | ZCCHC2 | U |
| 3986 | chr12 | 125204469 | 125204695 | Blood Monocytes + Macrophages | 0.3 | 0.89 | Intergenic | SCARB1 | U |
| 3987 | chr1 | 179310040 | 179310423 | Blood Monocytes + Macrophages | 0.34 | 0.93 | exon | SOATI | U |
| 3988 | chr7 | 6395854 | 6396328 | Blood Monocytes + Macrophages | 0.26 | 0.85 | Intergenic | FAM220A | U |
| 3989 | chr7 | 157562675 | 157562752 | Blood Monocytes + Macrophages | 0.2 | 0.78 | intron | PTPRN2 | U |
| 3990 | chr18 | 48356987 | 48357385 | Blood Monocytes + Macrophages | 0.34 | 0.91 | Intergenic | MRO | U |
| 3991 | chr5 | 132098766 | 132099123 | Blood Monocytes + Macrophages | 0.34 | 0.9 | intron | SEPT8 | U |
| 3992 | chr6 | 534481573 | 53448534 | Blood Monocytes + Macrophages | 0.36 | 0.92 | Intergenic | GCLC | U |
| 3993 | chr15 | 67159291 | 67159476 | Blood Monocytes + Macrophages | 0.37 | 0.92 | Intergenic | SMAD6 | U |
| 3994 | chr22 | 45687903 | 45688141 | Blood Monocytes + Macrophages | 0.27 | 0.8 | intron | UPK3A | U |
| 3995 | chr5 | 1697920781 | 169792341 | Blood Monocytes + Macrophages | 0.3 | 0.83 | intron | KCNIP1 | U |
| 3996 | chr5 | 154403647 | 154403723 | Blood Monocytes + Macrophages | 0.34 | 0.86 | Intergenic | KIF48 | U |
| 3997 | chr1 | 27315925 | 27316263 | Blood Monocytes + Macrophages | 0.37 | 0.85 | Intergenic | TRNP1 | U |
| 3998 | chr2 | 25503391 | 25503476 | Blood Monocytes + Macrophages | 0.1 | 0.88 | TTS | DNMT3A | U |
| 3999 | chr20 | 57493774 | 57493934 | Blood Monocytes + Macrophages | 0.12 | 0.86 | Intergenic | GNAS | U |
| 4000 | chr3 | 37814257 | 37814515 | Blood Monocytes + Macrophages | 0.27 | 0.86 | intron | ITGA9 | U |
| 4001 | chr6 | 26545988 | 26546169 | Blood Monocytes + Macrophages | 0.17 | 0.91 | exon | HMGN4 | U |
| 4002 | chr9 | 112918684 | 112918832 | Blood Monocytes + Macrophages | 0.26 | 0.92 | exor | AKAP2 | U |
| 4003 | chr12 | 26513019 | 26513305 | Blood Monocytes + Macrophages | 0.12 | 0.9 | intron | ITPR2 | U |
| 4004 | chr14 | 77984470 | 77984510 | Blood Monocytes + Macrophages | 0.16 | 0.91 | exon | SPTLC2 | U |
| 4005 | chr14 | 65675233 | 65675596 | Blood Monocytes + Macrophages | 0.19 | 0.89 | Intergenic | MAX | U |
| 4006 | chr5 | 2375895 | 2376032 | Blood Monocytes + Macrophages | 0.21 | 0.89 | intron | LOC100508120 | U |
| 4007 | chr22 | 39725254 | 39725514 | Blood Monocytes + Macrophages | 0.18 | 0.85 | Intergenic | RPL3 | U |
| 4008 | chr19 | 4666898 | 4667172 | Blood Monocytes + Macrophages | 0.28 | 0.92 | intron | C19orf10 | U |
| 4009 | chr6 | 15223330 | 15223449 | Blood Monocytes + Macrophages | 0.26 | 0.88 | Intergenic | JARID2 | U |
| 4010 | chr22 | 18243005 | 18243175 | Blood Monocytes + Macrophages | 0.26 | 0.88 | intron | BID | U |
| 4011 | chr22 | 40830751 | 40831106 | Blood Monocytes + Macrophages | 0.31 | 0.91 | intron | MKL1 | U |
| 4012 | chr20 | 20380706 | 20381076 | Blood Monocytes + Macrophages | 0.31 | 0.9 | intron | RALGAPA2 | U |
| 4013 | chr5 | 36564952 | 36565103 | Blood Monocytes + Macrophages | 0.72 | 0.16 | intron | SRSF3 | M |
| 4014 | chr8 | 22734960 | 22735112 | Blood Monocytes + Macrophages | 0.75 | 0.2 | intron | PEBP4 | M |
| 4015 | chr2 | 242810850 | 242811150 | Blood Monocytes + Macrophages | 0.7 | 0.19 | promoter-TSS | CXXC11 | M |
| 4016 | chr19 | 17185295 | 17185540 | Blood Monocytes + Macrophages | 0.61 | 0.12 | intron, promoter-TS [symbol] | HAUS8, HAUS8 | M |
| 4017 | chr7 | 99868422 | 99868626 | Blood Monocytes + Macrophages | 0.57 | 0.1 | intron | GATS | M |
| 4018 | chr6 | 143167094 | 143167322 | Blood Monocytes + Macrophages | 0.53 | 0.06 | intron | HIVEP2 | M |
| 4019 | chr17 | 1300733 | 1301023 | Blood Monocytes + Macrophages | 0.66 | 0.19 | Intron | YWHAE | M |
| 4020 | chr4 | 186950939 | 186951408 | Blood Monocytes + Macrophages | 0.6 | 0.14 | Intergenic | TLR3 | M |
| 4021 | chr17 | 7588787 | 7588920 | Blood Monocytes + Macrophages | 0.64 | 0.19 | promoter-TSS | WRAP53 | M |
| 4022 | chr20 | 60204143 | 60204393 | Blood Monocytes + Macrophages | 0.62 | 0.18 | intron | CDH4 | M |
| 4023 | chr11 | 661045631 | 66104621 | Blood Monocytes + Macrophages | 0.63 | 0.2 | promoter-TSS | RIN1 | M |
| 4024 | chr2 | 167350575 | 167350706 | Blood Monocytes + Macrophages | 0.57 | 0.14 | Intergenic | SCN7A | M |
| 4025 | chr10 | 133879665 | 133879805 | Blood Monocytes + Macrophages | 0.57 | 0.15 | Intergenic | JAKMIP3 | M |
| 4026 | chr9 | 140023480 | 140023651 | Blood Monocytes + Macrophages | 0.55 | 0.13 | Intergenic | GRIN1 | M |
| 4027 | chr10 | 43760488 | 43760706 | Blood Monocytes + Macrophages | 0.56 | 0.15 | intron | RASGEF1A | M |
| 4028 | chr16 | 21059351 | 21059646 | Blood Monocytes + Macrophages | 0.58 | 0.17 | intron | DNAH3 | M |
| 4029 | chr2 | 73496873 | 73497078 | Blood Monocytes + Macrophages | 0.55 | 0.15 | promoter-TSS | FBXO41 | M |
| 4030 | chr10 | 134256701 | 13425777 | Blood Monocytes + Macrophages | 0.54 | 0.16 | Intergenic | SEPHS1 | M |
| 4031 | chr19 | 17712257 | 17712367 | Blood Monocytes + Macrophages | 0.59 | 0.21 | exon, TTS | UNC13A, UNC13A | M |
| 4032 | chr3 | 161632615 | 161632867 | Blood Monocytes + Macrophages | 0.55 | 0.19 | Intergenic | OTOL1 | M |
| 4033 | chr19 | 13413607 | 13413705 | Blood Monocytes + Macrophages | 0.62 | 0.27 | intron | CACNA1A | M |
| 4034 | chr19 | 32329465 | 32329599 | Blood Monocytes + Macrophages | 0.55 | 0.2 | Intergenic | THEG5 | M |
| 4035 | chr13 | 46219611 | 46219885 | Blood Monocytes + Macrophages | 0.67 | 0.33 | Intergenic | FAM194B | M |
| 4036 | chr19 | 55877489 | 55877514 | Blood Monocytes + Macrophages | 0.68 | 0.35 | exon | IL11 | M |
| 4037 | chr22 | 19949901 | 19950167 | Blood Monocytes + Macrophages | 0.68 | 0.14 | promoter-TSS | COMT | M |
| 4038 | chr15 | 91370195 | 91370327 | Blood NK cells | 0.04 | 0.93 | Intergenic | FURIN | U |
| 4039 | chr17 | 72464526 | 72464888 | Blood NK cells | 0.07 | 0.93 | intron | CD300A | U |
| 4040 | chr3 | 17196544 | 17196765 | Blood NK cells | 0.07 | 0.92 | Intergenic | MIR3714 | U |
| 4041 | chr12 | 9983597 | 9983989 | Blood NK cells | 0.07 | 0.92 | intron | KLRF1 | U |
| 4042 | chr6 | 170426268 | 170426366 | Blood NK cells | 0.04 | 0.89 | Intergenic | LOC154449 | U |
| 4043 | chr17 | 724642343 | 72464452 | Blood NK cells | 0.09 | 0.91 | intron | CD300A | U |
| 4044 | chr18 | 77391680 | 77392004 | Blood NK cells | 0.08 | 0.9 | Intergenic | CTDP1 | U |
| 4045 | chr17 | 72460470 | 72460657 | Blood NK cells | 0.09 | 0.89 | Intergenic | CD300A | U |
| 4046 | chr19 | 30167085 | 30167286 | Blood NK cells | 0.1 | 0.9 | TTS | PLEKHF1 | U |
| 4047 | chr7 | 2039945 | 2040189 | Blood NK cells | 0.15 | 0.94 | intron | MAD1L1 | U |
| 4048 | chr6 | 166748511 | 166748769 | Blood NK cells | 0.15 | 0.93 | intron | SFT2D1 | U |
| 4049 | chr17 | 20601541 | 20601800 | Blood NK cells | 0.14 | 0.92 | Intergenic | LOC100287072 | U |
| 4050 | chr13 | 110411405 | 110411560 | Blood NK cells | 0.13 | 0.9 | intron | IRS2 | U |
| 4051 | chr7 | 1983823 | 1983918 | Blood NK cells | 0.16 | 0.88 | intron | MAD1L1 | U |
| 4052 | chr17 | 9465579 | 9465717 | Blood NK cells | 0.23 | 0.95 | intron | STX8 | U |
| 4053 | chr1 | 46386901 | 46387052 | Blood NK cells | 0.22 | 0.94 | intron | MAST2 | U |
| 4054 | chr3 | 32945527 | 32945860 | Blood NK cells | 0.22 | 0.93 | Intergenic | CCR4 | U |
| 4055 | chr17 | 55652681 | 55652809 | Blood NK cells | 0.26 | 0.93 | intron | MSI2 | U |
| 4056 | chr17 | 81012325 | 81012656 | Blood NK cells | 0.22 | 0.89 | Intergenic | B3GNTL1 | U |
| 4057 | chr3 | 32604098 | 32604525 | Blood NK cells | 0.27 | 0.94 | intron | DYNC1LI1 | U |
| 4058 | chr12 | 53762133 | 53762556 | Blood NK cells | 0.3 | 0.92 | Intergenic | SP1 | U |
| 4059 | chr2 | 68596508 | 68596737 | Blood NK cells | 0.29 | 0.9 | intron | PLEK | U |
| 4060 | chr2 | 225961049 | 225961156 | Blood NK cells | 0.34 | 0.93 | Intergenic | DOCK10 | U |
| 4061 | chr19 | 16483480 | 16483937 | Blood NK cells | 0.36 | 0.92 | intron | EPS15L1 | U |
| 4062 | chr7 | 100734328 | 100734388 | Blood NK cells | 0.05 | 0.95 | TTS | TRIM56 | U |
| 4063 | chr2 | 8423040 | 8423139 | Blood NK cells | 0.03 | 0.92 | intron | LINC00299 | U |
| 4064 | chr2 | 8595989 | 8596120 | Blood NK cells | 0.06 | 0.93 | Intergenic | LINC00299 | U |
| 4065 | chr6 | 35491881 | 35492060 | Blood NK cells | 0.05 | 0.92 | Intergenic | TULP1 | U |
| 4066 | chr1 | 1674160883 | 167416227 | Blood NK cells | 0.04 | 0.89 | intron | CD247 | U |
| 4067 | chr1 | 178380889 | 178381054 | Blood NK cells | 0.06 | 0.91 | intron | RASAL2 | U |
| 4068 | chr1 | 44059026 | 44059320 | Blood NK cells | 0.08 | 0.93 | intron | PTPRF | U |
| 4069 | chr13 | 110386260 | 110386324 | Blood NK cells | 0.11 | 0.95 | Intergenic | IRS2 | U |
| 4070 | chr2 | 240617639 | 240617769 | Blood NK cells | 0.09 | 0.93 | Intergenic | LOC150935 | U |
| 4071 | chr10 | 7285284 | 7285392 | Blood NK cells | 0.08 | 0.91 | intron | SFMBT2 | U |
| 4072 | chr11 | 333161 | 333286 | Blood NK cells | 0.08 | 0.9 | Intergenic | IFITM3 | U |
| 4073 | chr11 | 119767286 | 119767429 | Blood NK cells | 0.04 | 0.86 | Intergenic | PVRL1 | U |
| 4074 | chr11 | 1019378 | 1019547 | Blood NK cells | 0.07 | 0.89 | exon | MUC6 | U |
| 4075 | chr11 | 113945043 | 113945266 | Blood NK cells | 0.07 | 0.89 | intron | ZBTB16 | U |
| 4076 | chr9 | 139853947 | 139854192 | Blood NK cells | 0.06 | 0.88 | Intergenic | LCN12 | U |
| 4077 | chr9 | 127387483 | 127387755 | Blood NK cells | 0.11 | 0.93 | intron | NR6A1 | U |
| 4078 | chr1 | 167448709 | 167448982 | Blood NK cells | 0.07 | 0.89 | intron | CD247 | U |
| 4079 | chr7 | 80505151 | 80505424 | Blood NK cells | 0.06 | 0.88 | intron | SEMA3C | U |
| 4080 | chr16 | 88719942 | 88720407 | Blood NK cells | 0.07 | 0.89 | intron | MVD | U |
| 4081 | chr14 | 99917863 | 99917989 | Blood NK cells | 0.12 | 0.93 | intron | SETD3 | U |
| 4082 | chr20 | 50122196 | 50122404 | Blood NK cells | 0.09 | 0.9 | intron | NFATC2 | U |
| 4083 | chr6 | 170771086 | 170771501 | Blood NK cells | 0.09 | 0.9 | Intergenic | PSMB1 | U |
| 4084 | chr9 | 126308447 | 126308566 | Blood NK cells | 0.09 | 0.89 | intron | DENND1A | U |
| 4085 | chr8 | 28639413 | 28639559 | Blood NK cells | 0.15 | 0.95 | intron | INTS9 | U |
| 4086 | chr17 | 3608369 | 3608604 | Blood NK cells | 0.1 | 0.9 | Intergenic | P2RX5 | U |
| 4087 | chr12 | 680633263 | 68063567 | Blood NK cells | 0.12 | 0.92 | Intergenic | DYRK2 | U |
| 4088 | chr2 | 30517023 | 30517292 | Blood NK cells | 0.12 | 0.92 | Intergenic | LBH | U |
| 4089 | chr14 | 93006257 | 93006373 | Blood NK cells | 0.04 | 0.83 | intron | RIN3 | U |
| 4090 | chr16 | 358740 | 359147 | Blood NK cells | 0.14 | 0.93 | intron | AXIN1 | U |
| 4091 | chr16 | 85676577 | 85676678 | Blood NK cells | 0.1 | 0.88 | intron | GSE1 | U |
| 4092 | chr16 | 57682411 | 57682704 | Blood NK cells | 0.1 | 0.88 | intron | GPR56 | U |
| 4093 | chr11 | 870099563 | 87010087 | Blood NK cells | 0.18 | 0.95 | intron | TMEM135 | U |
| 4094 | chr22 | 43651426 | 43651560 | Blood NK cells | 0.07 | 0.84 | intron | SCUBE1 | U |
| 4095 | chr1 | 42362308 | 42362481 | Blood NK cells | 0.18 | 0.94 | intron | HIVEP3 | U |
| 4096 | chr5 | 10377133 | 10377320 | Blood NK cells | 0.19 | 0.95 | intron | MARCH6 | U |
| 4097 | chr17 | 45777414 | 45777900 | Blood NK cells | 0.16 | 0.92 | intron | TBKBP1 | U |
| 4098 | chr16 | 88720466 | 38720591 | Blood NK cells | 0.13 | 0.88 | intron | MVD | U |
| 4099 | chr16 | 297118 | 297331 | Blood NK cells | 0.15 | 0.9 | intron | ITFG3 | U |
| 4100 | chr16 | 85676406 | 85676565 | Blood NK cells | 0.1 | 0.84 | intron | GSE1 | U |
| 4101 | chr17 | 45817373 | 45817535 | Blood NK cells | 0.19 | 0.93 | intron | TBX21 | U |
| 4102 | chr17 | 80264260 | 80264504 | Blood NK cells | 0.17 | 0.91 | Intergenic | CD7 | U |
| 4103 | chr1 | 10585743 | 10585973 | Blood NK cells | 0.22 | 0.95 | intron | PEX14 | U |
| 4104 | chr8 | 124111382 | 124111562 | Blood NK cells | 0.21 | 0.93 | intron | WDR67 | U |
| 4105 | chr11 | 3161061 | 3161276 | Blood NK cells | 0.18 | 0.9 | intron | OSBPL5 | U |
| 4106 | chr3 | 57639835 | 57640177 | Blood NK cells | 0.21 | 0.93 | intron | DENND6A | U |
| 4107 | chr2 | 239314826 | 239315232 | Blood NK cells | 0.21 | 0.93 | Intergenic | ASB1 | U |
| 4108 | chr15 | 26047563 | 26047669 | Blood NK cells | 0.12 | 0.83 | intron | ATP10A | U |
| 4109 | chr22 | 466351223 | 46635299 | Blood NK cells | 0.19 | 0.9 | exon | PPARA | U |
| 4110 | chr17 | 65238302 | 65238485 | Blood NK cells | 0.2 | 0.91 | intron | HELZ | U |
| 4111 | chr7 | 32086031 | 3208890 | Blood NK cells | 0.16 | 0.87 | Intergenic | CARD11 | U |
| 4112 | chr19 | 12663796 | 12663860 | Blood NK cells | 0.21 | 0.91 | Intergenic | ZNF564 | U |
| 4113 | chr7 | 1983944 | 1984039 | Blood NK cells | 0.19 | 0.89 | intron | MAD1L1 | U |
| 4114 | chr16 | 81885018 | 81885221 | Blood NK cells | 0.22 | 0.92 | intron | PLOG2 | U |
| 4115 | chr16 | 4530035 | 4530345 | Blood NK cells | 0.21 | 0.91 | intron | HMOX2 | U |
| 4116 | chr2 | 420847 | 421203 | Blood NK cells | 0.22 | 0.92 | Intergenic | FAM150B | U |
| 4117 | chr6 | 161680430 | 161680823 | Blood NK cells | 0.25 | 0.95 | intron | AGPAT4 | U |
| 4118 | chr17 | 72468723 | 72469129 | Blood NK cells | 0.22 | 0.91 | intron | CD300A | U |
| 4119 | chr11 | 60891585 | 60892045 | Blood NK cells | 0.23 | 0.92 | intron | CD5 | U |
| 4120 | chr15 | 20817606 | 20817774 | Blood NK cells | 0.14 | 0.81 | Intergenic | GOLGA8CP | U |
| 4121 | chr3 | 9898214 | 9898473 | Blood NK cells | 0.23 | 0.9 | Intergenic | RPUSD3 | U |
| 4122 | chr16 | 29121968 | 29122251 | Blood NK cells | 0.2 | 0.87 | intron | RRN3P2 | U |
| 4123 | chr21 | 45430004 | 45430363 | Blood NK cells | 0.24 | 0.91 | Intergenic | TRAPPC10 | U |
| 4124 | chr3 | 15316508 | 15316951 | Blood NK cells | 0.21 | 0.88 | intron | SH3BP5 | U |
| 4125 | chr5 | 78839248 | 78839357 | Blood NK cells | 0.27 | 0.93 | Intergenic | HOMER1 | U |
| 4126 | chr12 | 102444277 | 102444421 | Blood NK cells | 0.26 | 0.92 | intron | CCDC53 | U |
| 4127 | chr17 | 779775431 | 77977704 | Blood NK cells | 0.26 | 0.92 | intron | TBC1D16 | U |
| 4128 | chr16 | 11331723 | 11332124 | Blood NK cells | 0.28 | 0.94 | Intergenic | SOCS1 | U |
| 4129 | chr5 | 159701203 | 159701619 | Blood NK cells | 0.21 | 0.87 | intron | CCNJL | U |
| 4130 | chr12 | 295321 | 295414 | Blood NK cells | 0.19 | 0.84 | Intergenic | SLC6A12 | U |
| 4131 | chr19 | 196380771 | 19638256 | Blood NK cells | 0.27 | 0.92 | exon | NDUFA13 | U |
| 4132 | chr19 | 2551962 | 2552155 | Blood NK cells | 0.23 | 0.88 | intron | GNG7 | U |
| 4133 | chr19 | 3799711 | 3799957 | Blood NK cells | 0.26 | 0.91 | intron | MATK | U |
| 4134 | chr8 | 123959972 | 123960220 | Blood NK cells | 0.3 | 0.95 | intron | ZHX2 | U |
| 4135 | chr17 | 45792622 | 45792988 | Blood NK cells | 0.22 | 0.87 | Intergenic | TBX21 | U |
| 4136 | chr11 | 128670964 | 128671386 | Blood NK cells | 0.27 | 0.92 | intron | FLI1 | U |
| 4137 | chr18 | 74830766 | 74831235 | Blood NK cells | 0.3 | 0.94 | intron | MBP | U |
| 4138 | chr19 | 18805212 | 18805698 | Blood NK cells | 0.3 | 0.93 | intron | CRTC1 | U |
| 4139 | chr2 | 105691355 | 105691759 | Blood NK cells | 0.34 | 0.96 | intron | MRPS9 | U |
| 4140 | chr18 | 54240473 | 54240658 | Blood NK cells | 0.31 | 0.92 | Intergenic | TXNL1 | U |
| 4141 | chr15 | 234849903 | 23485384 | Blood NK cells | 0.21 | 0.82 | Intergenic | GOLGA8EP | U |
| 4142 | chr7 | 132942682 | 132943008 | Blood NK cells | 0.33 | 0.93 | intron | EXOC4 | U |
| 4143 | chr21 | 34575182 | 34575275 | Blood NK cells | 0.31 | 0.89 | Intergenic | IFNAR2 | U |
| 4144 | chr18 | 74828467 | 74828674 | Blood NK cells | 0.35 | 0.93 | intron | MBP | U |
| 4145 | chr15 | 69088450 | 69088578 | Blood NK cells | 0.33 | 0.89 | intron | ANP32A | U |
| 4146 | chr7 | 104995335 | 104995830 | Blood NK cells | 0.35 | 0.9 | intron | SRPK2 | U |
| 4147 | chr3 | 255027721 | 25503190 | Blood NK cells | 0.09 | 0.92 | intron | RARB | U |
| 4148 | chr1 | 3278151 | 3278603 | Blood NK cells | 0.33 | 0.9 | intron | PRDM16 | U |
| 4149 | chr20 | 5977891 | 5978014 | Blood NK cells | 0.11 | 0.93 | Intergenic | CRLS1 | U |
| 4150 | chr14 | 25147832 | 25147903 | Blood NK cells | 0.06 | 0.89 | Intergenic | GZMB | U |
| 4151 | chr6 | 15505860 | 15505967 | Blood NK cells | 0.03 | 0.86 | intron | JARID2 | U |
| 4152 | chr6 | 15505986 | 15506113 | Blood NK cells | 0.02 | 0.83 | intron | JARID2 | U |
| 4153 | chr19 | 13997072 | 13997271 | Blood NK cells | 0.11 | 0.9 | intron | C19orf57 | U |
| 4154 | chr14 | 56677405 | 56677888 | Blood NK cells | 0.1 | 0.87 | intron | PELI2 | U |
| 4155 | chr14 | 35210909 | 35210977 | Blood NK cells | 0.13 | 0.88 | Intergenic | CFL2 | U |
| 4156 | chr14 | 25101099 | 25101378 | Blood NK cells | 0.18 | 0.91 | exon | GZMB | U |
| 4157 | chr9 | 27676350 | 27676628 | Blood NK cells | 0.2 | 0.88 | Intergenic | C9orf72 | U |
| 4158 | chr20 | 25327048 | 25327427 | Blood NK cells | 0.25 | 0.93 | intron | ABHD12 | U |
| 4159 | chr2 | 45926949 | 45927238 | Blood NK cells | 0.26 | 0.93 | intron | PRKCE | U |
| 4160 | chr20 | 43612904 | 43613139 | Blood NK cells | 0.28 | 0.94 | intron | STK4 | U |
| 4161 | chr9 | 113684077 | 113684327 | Blood NK cells | 0.25 | 0.88 | intron | PAR1 | U |
| 4162 | chr14 | 25096504 | 25096619 | Blood NK cells | 0.29 | 0.91 | Intergenic | GZMB | U |
| 4163 | chr3 | 126193438 | 126193625 | Blood NK cells | 0.68 | 0.16 | intron | ZXDC | M |
| 4164 | chr4 | 186950939 | 186951408 | Blood NK cells | 0.64 | 0.13 | Intergenic | TLR3 | M |
| 4165 | chr12 | 50497589 | 50497828 | Blood NK cells | 0.59 | 0.09 | exon | GPD1 | M |
| 4166 | chr19 | 1860062 | 1860310 | Blood NK cells | 0.64 | 0.14 | intron | KLF16 | M |
| 4167 | chr9 | 130741664 | 130742038 | Blood NK cells | 0.55 | 0.06 | intron | FAM102A | M |
| 4168 | chr1 | 93249291 | 93249657 | Blood NK cells | 0.62 | 0.14 | intron | EVI5 | M |
| 4169 | chr19 | 49371794 | 49371940 | Blood NK cells | 0.59 | 0.12 | exon, promoter-TSS | PLEKHA4, PLEKHA4 | M |
| 4170 | chr3 | 49396303 | 49396392 | Blood NK cells | 0.7 | 0.24 | promoter-TSS | GPX1 | M |
| 4171 | chr11 | 72392383 | 72392660 | Blood NK cells | 0.59 | 0.13 | Intergenic | PDE2A | M |
| 4172 | chr16 | 790134 | 790306 | Blood NK cells | 0.6 | 0.15 | intron | NARFL | M |
| 4173 | chr12 | 50017659 | 50017936 | Blood NK cells | 0.63 | 0.18 | exon | PRPF40B | M |
| 4174 | chr2 | 88925612 | 88925997 | Blood NK cells | 0.53 | 0.08 | intron | EIF2AK3 | M |
| 4175 | chr7 | 100304423 | 100304582 | Blood NK cells | 0.53 | 0.09 | exon | POP7 | M |
| 4176 | chr16 | 11034436 | 11034739 | Blood NK cells | 0.67 | 0.23 | intron | DEXI | M |
| 4177 | chr17 | 20811492 | 20811549 | Blood NK cells | 0.59 | 0.16 | Intergenic | CCDC144NL | M |
| 4178 | chr3 | 10053303 | 10053557 | Blood NK cells | 0.6 | 0.18 | promoter-TSS | LOC401052 | M |
| 4179 | chr7 | 101448278 | 101448507 | Blood NK cells | 0.56 | 0.15 | Intergenic | CUX1 | M |
| 4180 | chr17 | 7740472 | 7740848 | Blood NK cells | 0.53 | 0.12 | Intergenic | KDM68 | M |
| 4181 | chr1 | 45451434 | 45451503 | Blood NK cells | 0.52 | 0.12 | intron | EIF283 | M |
| 4182 | chr7 | 156744110 | 156744441 | Blood NK cells | 0.56 | 0.16 | intron | NOM1 | M |
| 4183 | chr11 | 73358873 | 73358989 | Blood NK cells | 0.59 | 0.2 | promoter-TSS | PLEKHB1 | M |
| 4184 | chrX | 47508721 | 47508942 | Blood NK cells | 0.6 | 0.25 | intron | ELK1 | M |
| 4185 | chr14 | 35590322 | 35590444 | Blood NK cells | 0.68 | 0.17 | intron | PPP2R3C | M |
| 4186 | chr22 | 39919480 | 39919725 | Blood NK cells | 0.62 | 0.11 | TTS | ATF4 | M |
| 4187 | chr20 | 62340144 | 62340242 | Blood NK cells | 0.59 | 0.21 | promoter-TSS | ARFRP1 | M |
| 4188 | chr5 | 118635509 | 118635757 | Blood T cells | 0.02 | 0.91 | intron | TNFAIP8 | U |
| 4189 | chr7 | 3501574 | 3501867 | Blood T cells | 0.06 | 0.92 | intron | SDK1 | U |
| 4190 | chr1 | 32718447 | 32718737 | Blood T cells | 0.03 | 0.88 | intron | LCK | U |
| 4191 | chr11 | 118176757 | 118177142 | Blood T cells | 0.05 | 0.89 | intron | CD3E | U |
| 4192 | chr16 | 68244845 | 68245279 | Blood T cells | 0.09 | 0.92 | intron | NFATC3 | U |
| 4193 | chr11 | 60739310 | 60739437 | Blood T cells | 0.03 | 0.86 | exon | CD6 | U |
| 4194 | chr19 | 6362839 | 6362914 | Blood T cells | 0.09 | 0.9 | intron | CLPP | U |
| 4195 | chr11 | 118213527 | 118214010 | Blood T cells | 0.06 | 0.87 | promoter-TSS | CD3D | U |
| 4196 | chr1 | 12237782 | 12237917 | Blood T cells | 0.08 | 0.86 | intron | TNFRSF1B | U |
| 4197 | chr10 | 129131921 | 129132266 | Blood T cells | 0.15 | 0.92 | intron | DOCK1 | U |
| 4198 | chr2 | 205108059 | 205108161 | Blood T cells | 0.13 | 0.89 | Intergenic | PARD38 | U |
| 4199 | chr19 | 7225929 | 7226136 | Blood T cells | 0.05 | 0.81 | intron | INSR | U |
| 4200 | chr22 | 48475730 | 48475916 | Blood T cells | 0.1 | 0.85 | Intergenic | MIR3201 | U |
| 4201 | chr12 | 69285539 | 69285673 | Blood T cells | 0.17 | 0.9 | intron | CPM | U |
| 4202 | chr17 | 71818566 | 71818917 | Blood T cells | 0.18 | 0.91 | intron | LINC00469 | U |
| 4203 | chr12 | 47605685 | 47606094 | Blood T cells | 0.23 | 0.94 | intron | PCED1B-ASI | U |
| 4204 | chr2 | 85667088 | 85667541 | Blood T cells | 0.21 | 0.91 | Intergenic | SH2D6 | U |
| 4205 | chr3 | 197596061 | 197596302 | Blood T cells | 0.26 | 0.94 | intron | LRCH3 | U |
| 4206 | chr3 | 111261184 | 111261528 | Blood T cells | 0.04 | 0.89 | intron | CD96 | U |
| 4207 | chr1 | 206976573 | 206976899 | Blood T cells | 0.01 | 0.85 | intron | IL19 | U |
| 4208 | chr13 | 40761258 | 40761586 | Blood T cells | 0.04 | 0.88 | intron | LINC00332 | U |
| 4209 | chr1 | 32718801 | 32719173 | Blood T cells | 0.05 | 0.89 | intron | LCK | U |
| 4210 | chr5 | 156620811 | 156621258 | Blood T cells | 0.01 | 0.85 | intron | ITK | U |
| 4211 | chr11 | 60870283 | 60870687 | Blood T cells | 0.04 | 0.87 | intron | CD5 | U |
| 4212 | chr15 | 38945624 | 38945753 | Blood T cells | 0.07 | 0.89 | Intergenic | C15orf53 | U |
| 4213 | chr2 | 175354316 | 175354510 | Blood T cells | 0.09 | 0.91 | Intergenic | GPR155 | U |
| 4214 | chr2 | 234176989 | 234177299 | Blood T cells | 0.04 | 0.86 | intron | ATG16L1 | U |
| 4215 | chr11 | 77914232 | 77914342 | Blood T cells | 0.11 | 0.92 | intron | USP35 | U |
| 4216 | chr5 | 1186350981 | 118635285 | Blood T cells | 0.12 | 0.93 | intron | TNFAIP8 | U |
| 4217 | chr4 | 873212 | 873550 | Blood T cells | 0.07 | 0.88 | intron | GAK | U |
| 4218 | chr1 | 39443248 | 39443657 | Blood T cells | 0.08 | 0.89 | Intergenic | AKIRIN1 | U |
| 4219 | chr2 | 86159584 | 86159709 | Blood T cells | 0.08 | 0.88 | Intergenic | ST3GAL5 | U |
| 4220 | chr5 | 118635784 | 118636033 | Blood T cells | 0.13 | 0.93 | intron | TNFAIP8 | U |
| 4221 | chr5 | 39198919 | 39199198 | Blood T cells | 0.1 | 0.9 | intron | FY8 | U |
| 4222 | chr10 | 27390494 | 27390837 | Blood T cells | 0.06 | 0.86 | Intergenic | ANKRD26 | U |
| 4223 | chr13 | 48760259 | 48760363 | Blood T cells | 0.08 | 0.87 | Intergenic | ITM2B | U |
| 4224 | chr22 | 50232991 | 50233298 | Blood T cells | 0.09 | 0.88 | Intergenic | ZBED4 | U |
| 4225 | chr1 | 1172802991 | 117280524 | Blood T cells | 0.09 | 0.87 | Intergenic | CD2 | U |
| 4226 | chr11 | 60870871 | 60871101 | Blood T cells | 0.04 | 0.82 | intron | CD5 | U |
| 4227 | chr10 | 128899365 | 128899644 | Blood T cells | 0.13 | 0.91 | intron | DOCK1 | U |
| 4228 | chr3 | 45984836 | 45985169 | Blood T cells | 0.13 | 0.91 | promoter-TSS | CXCR6 | U |
| 4229 | chr7 | 1506954003 | 150695536 | Blood T cells | 0.08 | 0.84 | exon | NOS3 | U |
| 4230 | chr3 | 195336331 | 195336579 | Blood T cells | 0.14 | 0.89 | Intergenic | APOD | U |
| 4231 | chr4 | 114604657 | 114605003 | Blood T cells | 0.12 | 0.87 | intron | CAMK2D | U |
| 4232 | chr20 | 48819424 | 48819564 | Blood T cells | 0.16 | 0.9 | Intergenic | CEBPB | U |
| 4233 | chr12 | 66788079 | 66788229 | Blood T cells | 0.09 | 0.83 | intron | GRIP1 | U |
| 4234 | chr2 | 64205129 | 64205490 | Blood T cells | 0.17 | 0.91 | intron | VPS54 | U |
| 4235 | chr10 | 11887167 | 11887647 | Blood T cells | 0.14 | 0.88 | intron | PROSER2 | U |
| 4236 | chr20 | 62270221 | 62270344 | Blood T cells | 0.14 | 0.87 | TTS | STMN3 | U |
| 4237 | chr5 | 80303344 | 80303560 | Blood T cells | 0.13 | 0.86 | intron | RASGRF2 | U |
| 4238 | chr5 | 169740386 | 169740677 | Blood T cells | 0.00 | 0.82 | Intergenic | LCP2 | U |
| 4239 | chr12 | 27654980 | 27655356 | Blood T cells | 0.16 | 0.89 | TTS | C12orf70 | U |
| 4240 | chr19 | 1079557 | 1079662 | Blood T cells | 0.1 | 0.82 | intron | HMHA1 | U |
| 4241 | chr1 | 11965595 | 11965703 | Blood T cells | 0.17 | 0.89 | Intergenic | KIAA2013 | U |
| 4242 | chr9 | 139005420 | 139005539 | Blood T cells | 0.17 | 0.89 | TTS | C9orf69 | U |
| 4243 | chr4 | 7856825 | 7857021 | Blood T cells | 0.14 | 0.86 | intron | AFAP1 | U |
| 4244 | chr5 | 133455511 | 133455735 | Blood T cells | 0.18 | 0.9 | intron | TCF7 | U |
| 4245 | chr11 | 11171265 | 11171698 | Blood T cells | 0.14 | 0.85 | Intergenic | CSNK2A3 | U |
| 4246 | chr6 | 37177349 | 37177491 | Blood T cells | 0.13 | 0.83 | Intergenic | PIM1 | U |
| 4247 | chr11 | 1178233701 | 117823587 | Blood T cells | 0.19 | 0.89 | Intergenic | TMPRSS13 | U |
| 4248 | chr11 | 676881943 | 67688672 | Blood T cells | 0.15 | 0.85 | Intergenic | UNC93B1 | U |
| 4249 | chr7 | 99221780 | 99222108 | Blood T cells | 0.2 | 0.89 | intron | ZSCAN25 | U |
| 4250 | chr15 | 78914218 | 78914285 | Blood T cells | 0.19 | 0.87 | promoter-TSS | CHRNA3 | U |
| 4251 | chr11 | 69153814 | 69153915 | Blood T cells | 0.21 | 0.89 | Intergenic | MYEOV | U |
| 4252 | chr5 | 142217501 | 14222178 | Blood T cells | 0.25 | 0.93 | intron | TRIO | U |
| 4253 | chr5 | 156616441 | 156616815 | Blood T cells | 0.22 | 0.89 | intron | ITK | U |
| 4254 | chr3 | 1285071603 | 128507648 | Blood T cells | 0.21 | 0.88 | intron | RAB7A | U |
| 4255 | chr17 | 2465833 | 2466048 | Blood T cells | 0.17 | 0.83 | Intergenic | PAFAH1B1 | U |
| 4256 | chrX | 16907400 | 16907708 | Blood T cells | 0.24 | 0.9 | Intergenic | RBBP7 | U |
| 4257 | chr17 | 75449254 | 75449558 | Blood T cells | 0.24 | 0.89 | intron | SEPT9 | U |
| 4258 | chr1 | 224906543 | 224906691 | Blood T cells | 0.24 | 0.88 | intron | CNIH3 | U |
| 4259 | chr7 | 99816908 | 99817259 | Blood T cells | 0.2 | 0.84 | intron | PVRIG | U |
| 4260 | chr6 | 39890453 | 39890917 | Blood T cells | 0.24 | 0.88 | intron | MOCS1 | U |
| 4261 | chr1 | 2949671 | 2949793 | Blood T cells | 0.25 | 0.88 | Intergenic | ACTRT2 | U |
| 4262 | chr2 | 1970209913 | 197021425 | Blood T cells | 0.27 | 0.9 | exor | STK17B | U |
| 4263 | chr6 | 91182911 | 91183254 | Blood T cells | 0.27 | 0.88 | Intergenic | MAP3K7 | U |
| 4264 | chr6 | 141887894 | 141888315 | Blood T cells | 0.27 | 0.88 | Intergenic | NMBR | U |
| 4265 | chr19 | 9919947 | 9920429 | Blood T cells | 0.28 | 0.89 | TTS, exon | FBXL12, FBXL12 | U |
| 4266 | chr4 | 10163370 | 10163839 | Blood T cells | 0.28 | 0.88 | Intergenic | WDR1 | U |
| 4267 | chr16 | 29033092 | 29033226 | Blood T cells | 0.29 | 0.87 | Intergenic | LAT | U |
| 4268 | chr15 | 58108937 | 58109125 | Blood T cells | 0.25 | 0.83 | Intergenic | POLR2M | U |
| 4269 | chr9 | 132653217 | 132653454 | Blood T cells | 0.33 | 0.91 | intron | FNBP1 | U |
| 4270 | chr15 | 22992596 | 22992896 | Blood T cells | 0.33 | 0.9 | intron | CYFIP1 | U |
| 4271 | chrX | 20372817 | 20372961 | Blood T cells | 0.22 | 0.78 | Intergenic | RPS6KA3 | U |
| 4272 | chr20 | 4359556 | 4359846 | Blood T cells | 0.33 | 0.89 | Intergenic | ADRA1D | U |
| 4273 | chr9 | 1356993141 | 135699800 | Blood T cells | 0.29 | 0.85 | intron | AK8 | U |
| 4274 | chr1 | 9252942 | 9253192 | Blood T cells | 0.23 | 0.77 | Intergenic | MIR34A | U |
| 4275 | chr11 | 1339282013 | 133928275 | Blood T cells | 0.07 | 0.89 | Intergenic | JAM3 | U |
| 4276 | chr1 | 226919824 | 226920198 | Blood T cells | 0.11 | 0.9 | intron | ITPKB | U |
| 4277 | chr3 | 28918821 | 28919041 | Blood T cells | 0.06 | 0.92 | Intergenic | HMBOX1 | U |
| 4278 | chr9 | 92096477 | 92096893 | Blood T cells | 0.06 | 0.89 | Intergenic | SEMA4D | U |
| 4279 | chr17 | 80084861 | 80085213 | Blood T cells | 0.1 | 0.92 | intron | CCDC57 | U |
| 4280 | chr14 | 22973391 | 22973496 | Blood T cells | 0.13 | 0.94 | Intergenic | DAD1 | U |
| 4281 | chr11 | 118214295 | 118214531 | Blood T cells | 0.09 | 0.89 | promoter-TSS | CD3G | U |
| 4282 | chr8 | 144543703 | 144543810 | Blood T cells | 0.28 | 0.91 | intron | ZC3H3 | U |
| 4283 | chr22 | 40297780 | 40297925 | Blood T cells | 0.02 | 0.9 | intron | GRAP2 | U |
| 4284 | chr17 | 80084613 | 80084816 | Blood T cells | 0.02 | 0.9 | intron | CCDC57 | U |
| 4285 | chr17 | 80084450 | 80084581 | Blood T cells | 0.07 | 0.94 | intron | CCDC57 | U |
| 4286 | chr7 | 138588277 | 138588505 | Blood T cells | 0.04 | 0.9 | exon | KIAA1549 | U |
| 4287 | chr14 | 61799217 | 61799678 | Blood T cells | 0.02 | 0.87 | intron | PRKCH | U |
| 4288 | chr1 | 8212967 | 8213183 | Blood T cells | 0.1 | 0.94 | Intergenic | ERRFI1 | U |
| 4289 | chr7 | 623458 | 623727 | Blood T cells | 0.07 | 0.91 | intron | PRKAR1B | U |
| 4290 | chr8 | 144543841 | 144544020 | Blood T cells | 0.07 | 0.9 | intron | ZC3H3 | U |
| 4291 | chr21 | 43825682 | 43825849 | Blood T cells | 0.11 | 0.93 | intron | UBASH3A | U |
| 4292 | chr22 | 40720366 | 40720634 | Blood T cells | 0.1 | 0.91 | exon | TNRC6B | U |
| 4293 | chr6 | 548666 | 548817 | Blood T cells | 0.11 | 0.9 | intron | EXOC2 | U |
| 4294 | chr9 | 132631612 | 132631844 | Blood T cells | 0.08 | 0.87 | intron | USP20 | U |
| 4295 | chr14 | 22965217 | 22965481 | Blood T cells | 0.15 | 0.94 | Intergenic | DAD1 | U |
| 4296 | chr14 | 69248340 | 69248721 | Blood T cells | 0.13 | 0.89 | Intergenic | ZFP36L1 | U |
| 4297 | chr9 | 97384575 | 97384723 | Blood T cells | 0.16 | 0.91 | intron | FBP1 | U |
| 4298 | chr21 | 43825906 | 43826269 | Blood T cells | 0.11 | 0.86 | intron | UBASH3A | U |
| 4299 | chr14 | 22983921 | 22984402 | Blood T cells | 0.12 | 0.87 | Intergenic | DAD1 | U |
| 4300 | chr14 | 91712322 | 91712592 | Blood T cells | 0.2 | 0.94 | intron | GPR68 | U |
| 4301 | chr7 | 138778366 | 138778558 | Blood T cells | 0.2 | 0.93 | intron | ZC3HAV1 | U |
| 4302 | chr22 | 37634189 | 37634518 | Blood T cells | 0.17 | 0.9 | intron | RAC2 | U |
| 4303 | chr13 | 114776475 | 114776550 | Blood T cells | 0.17 | 0.89 | intron | RASA3 | U |
| 4304 | chr22 | 40721118 | 40721588 | Blood T cells | 0.18 | 0.9 | exon | TNRC6B | U |
| 4305 | chr7 | 1864297 | 1864452 | Blood T cells | 0.13 | 0.84 | intron | MAD1L1 | U |
| 4306 | chr16 | 89163357 | 89163626 | Blood T cells | 0.2 | 0.89 | intron | ACSF3 | U |
| 4307 | chr19 | 1079683 | 1079785 | Blood T cells | 0.26 | 0.94 | intron | HMHA1 | U |
| 4308 | chr20 | 35273540 | 35274018 | Blood T cells | 0.23 | 0.91 | intron | SLA2 | U |
| 4309 | chr14 | 23017482 | 23017886 | Blood T cells | 0.22 | 0.89 | Intergenic | DAD1 | U |
| 4310 | chr14 | 22983517 | 22983907 | Blood T cells | 0.28 | 0.93 | Intergenic | DAD1 | U |
| 4311 | chr12 | 1334437263 | 133443869 | Blood T cells | 0.24 | 0.88 | intron | SCHER | U |
| 4312 | chr22 | 37627299 | 37627492 | Blood T cells | 0.23 | 0.86 | exon, intron | RAC2, RAC2 | U |
| 4313 | chr5 | 169407439 | 169407751 | Blood T cells | 0.89 | 0.07 | exon | FAM1968 | M |
| 4314 | chr10 | 92794681 | 92794868 | Blood T cells | 0.92 | 0.14 | Intergenic | LINC00502 | M |
| 4315 | chr19 | 18268001 | 18268236 | Blood T cells | 0.85 | 0.08 | intron | PIK3R2 | M |
| 4316 | chr6 | 108883848 | 108884146 | Blood T cells | 0.84 | 0.08 | intron | FOXO3 | M |
| 4317 | chr22 | 43390988 | 43391450 | Blood T cells | 0.81 | 0.09 | intron | PACSIN2 | M |
| 4318 | chr17 | 33825598 | 33825940 | Blood T cells | 0.88 | 0.22 | Intergenic | SLFN12L | M |
| 4319 | chr1 | 27855041 | 27855172 | Blood T cells | 0.64 | 0.04 | Intergenic | WASF2 | M |
| 4320 | chr12 | 51610762 | 51610956 | Blood T cells | 0.65 | 0.06 | intron | POU6F1 | M |
| 4321 | chr11 | 48001422 | 48001494 | Blood T cells | 0.69 | 0.11 | promoter-TSS | PTPRJ | M |
| 4322 | chr1 | 9352196 | 9352547 | Blood T cells | 0.58 | 0.08 | promoter-TSS | SPSB1 | M |
| 4323 | chr5 | 10695232 | 10695495 | Blood T cells | 0.66 | 0.03 | intron | DAP | M |
| 4324 | chr6 | 108883005 | 108883217 | Blood T cells | 0.91 | 0.07 | intron | FOXO3 | M |
| 4325 | chr6 | 108882762 | 108882982 | Blood T cells | 0.85 | 0.09 | exon | FOXO3 | M |
| 4326 | chr6 | 108883281 | 108883578 | Blood T cells | 0.87 | 0.11 | intron | FOXO3 | M |
| 4327 | chr3 | 170073717 | 170074014 | Blood T cells | 0.85 | 0.11 | Intergenic | SKIL | M |
| 4328 | chr1 | 24229215 | 24229320 | Blood T cells | 0.77 | 0.09 | intron | CNR2 | M |
| 4329 | chr6 | 12013731 | 12013888 | Blood T cells | 0.7 | 0.04 | intron | HIVEP1 | M |
| 4330 | chr10 | 88295324 | 88295707 | Blood T cells | 0.73 | 0.07 | Intergenic | WAPAL | M |
| 4331 | chr1 | 27928352 | 27928843 | Blood T cells | 0.69 | 0.04 | intron | AHDC1 | M |
| 4332 | chr18 | 46067903 | 45067968 | Blood T cells | 0.68 | 0.04 | intron | CTIF | M |
| 4333 | chr1 | 24229334 | 24229683 | Blood T cells | 0.64 | 0.03 | intron | CNR2 | M |
| 4334 | chr9 | 132806098 | 132806194 | Blood T cells | 0.65 | 0.06 | promoter-TSS | FNBP1 | M |
| 4335 | chr13 | 413642271 | 41364434 | Blood T cells | 0.62 | 0.03 | intron | SLC25A15 | M |
| 4336 | chr8 | 90770736 | 90770983 | Blood T cells | 0.64 | 0.05 | promoter-TSS, intron | RIPK2, RIPK2 | M |
| 4337 | chr22 | 21239949 | 21240108 | Blood T cells | 0.66 | 0.08 | intron | SNAP29 | M |
| 4338 | chr6 | 135513150 | 135513182 | Erythrocyte progenitor cells | 0.01 | 0.96 | intron | MYB | U |
| 4339 | chr16 | 31089524 | 31089734 | Erythrocyte progenitor cells | 0 | 0.95 | exon | ZNF646 | U |
| 4340 | chr19 | 13830749 | 13830838 | Erythrocyte progenitor cells | 0 | 0.94 | Intergenic | CCDC130 | U |
| 4341 | chr5 | 1452240223 | 145224183 | Erythrocyte progenitor cells | 0 | 0.94 | Intergenic | PRELID2 | U |
| 4342 | chr6 | 34583467 | 34583665 | Erythrocyte progenitor cells | 0 | 0.94 | intron | C6orf106 | U |
| 4343 | chr1 | 52541390 | 52541632 | Erythrocyte progenitor cells | 0 | 0.94 | intron | BTF3L4 | U |
| 4344 | chr16 | 566478 | 566642 | Erythrocyte progenitor cells | 0.01 | 0.94 | intron | RAB11FIP3 | U |
| 4345 | chr15 | 34946822 | 34947087 | Erythrocyte progenitor cells | 0.01 | 0.94 | Intergenic | GOLGA8B | U |
| 4346 | chr19 | 19396535 | 19396859 | Erythrocyte progenitor cells | 0.03 | 0.96 | intron | SUGP1 | U |
| 4347 | chr19 | 56045142 | 56045196 | Erythrocyte progenitor cells | 0.01 | 0.93 | intron | SBK2 | U |
| 4348 | chr8 | 41910906 | 41910992 | Erythrocyte progenitor cells | 0.01 | 0.93 | Intergenic | KAT6A | U |
| 4349 | chr12 | 113629175 | 113629398 | Erythrocyte progenitor cells | 0.02 | 0.94 | exon | C12orf52 | U |
| 4350 | chr9 | 1402835881 | 140283784 | Erythrocyte progenitor cells | 0.03 | 0.93 | intron | EXD3 | U |
| 4351 | chr16 | 449350 | 449653 | Erythrocyte progenitor cells | 0 | 0.9 | intron | NME4 | U |
| 4352 | chr9 | 139122369 | 139122429 | Erythrocyte progenitor cells | 0.05 | 0.95 | intron | QSOX2 | U |
| 4353 | chr15 | 85160527 | 85160623 | Erythrocyte progenitor cells | 0.06 | 0.96 | TTS | ZSCAN2 | U |
| 4354 | chr7 | 2369007 | 2369160 | Erythrocyte progenitor cells | 0.05 | 0.95 | Intergenic | SNX8 | U |
| 4355 | chr10 | 46070843 | 46071051 | Erythrocyte progenitor cells | 0.06 | 0.96 | intron | Mar-08 | U |
| 4356 | chr2 | 233922626 | 233922747 | Erythrocyte progenitor cells | 0.04 | 0.93 | Intergenic | INPP5D | U |
| 4357 | chr1 | 11975161 | 1197811 | Erythrocyte progenitor cells | 0.05 | 0.94 | intron | UBE2J2 | U |
| 4358 | chr7 | 100132911 | 100133067 | Erythrocyte progenitor cells | 0.06 | 0.94 | Intergenic | AGFG2 | U |
| 4359 | chr2 | 171834299 | 171834625 | Erythrocyte progenitor cells | 0.06 | 0.94 | Intergenic | GORASP2 | U |
| 4360 | chr16 | 21548067 | 21548198 | Erythrocyte progenitor cells | 0.11 | 0.95 | Intergenic | SLC7A5P2 | U |
| 4361 | chr11 | 43385285 | 43385628 | Erythrocyte progenitor cells | 0.12 | 0.95 | intron | TTC17 | U |
| 4362 | chr12 | 10854582 | 10854720 | Erythrocyte progenitor cells | 0 | 0.94 | intron | CSDA | U |
| 4363 | chr19 | 14253916 | 14254072 | Erythrocyte progenitor cells | 0 | 0.93 | intron | LOC100507373 | U |
| 4364 | chr2 | 37642469 | 37642704 | Erythrocyte progenitor cells | 0.01 | 0.93 | Intergenic | QPCT | U |
| 4365 | chr3 | 43719906 | 43720154 | Erythrocyte progenitor cells | 0.01 | 0.93 | Intergenic | ABHD5 | U |
| 4366 | chr19 | 6387724 | 6387972 | Erythrocyte progenitor cells | 0.01 | 0.93 | intron | GTF2F1 | U |
| 4367 | chr6 | 142733020 | 142733281 | Erythrocyte progenitor cells | 0.02 | 0.94 | intron | GPR126 | U |
| 4368 | chr8 | 41654193 | 41654456 | Erythrocyte progenitor cells | 0 | 0.91 | intron | ANK1 | U |
| 4369 | chr16 | 449680 | 449814 | Erythrocyte progenitor cells | 0 | 0.9 | exon, intron | NME4, NME4 | U |
| 4370 | chr8 | 41654461 | 41654661 | Erythrocyte progenitor cells | 0.01 | 0.91 | intron | ANK1 | U |
| 4371 | chr9 | 130603725 | 130603934 | Erythrocyte progenitor cells | 0 | 0.9 | intron | ENG | U |
| 4372 | chr3 | 98302871 | 98303126 | Erythrocyte progenitor cells | 0.01 | 0.91 | intron | CPOX | U |
| 4373 | chr1 | 230380975 | 230381265 | Erythrocyte progenitor cells | 0.05 | 0.95 | intron | GALNT2 | U |
| 4374 | chr5 | 141487592 | 141487910 | Erythrocyte progenitor cells | 0.05 | 0.95 | promoter-TSS | NDFIP1 | U |
| 4375 | chr6 | 160107983 | 160108315 | Erythrocyte progenitor cells | 0.01 | 0.91 | intron | SOD2 | U |
| 4376 | chr6 | 604475 | 604839 | Erythrocyte progenitor cells | 0.03 | 0.93 | intron | EXOC2 | U |
| 4377 | chr10 | 12067474 | 12067879 | Erythrocyte progenitor cells | 0.01 | 0.91 | intron | UPF2 | U |
| 4378 | chr1 | 155732093 | 155732567 | Erythrocyte progenitor cells | 0.04 | 0.94 | intron | GON4L | U |
| 4379 | chr2 | 9138738 | 9139229 | Erythrocyte progenitor cells | 0 | 0.9 | intron | MBOAT2 | U |
| 4380 | chr2 | 97584676 | 97584797 | Erythrocyte progenitor cells | 0.01 | 0.9 | intron | FAM1788 | U |
| 4381 | chr19 | 3194305 | 3194737 | Erythrocyte progenitor cells | 0.04 | 0.93 | intron | NCLN | U |
| 4382 | chr12 | 122640193 | 122640326 | Erythrocyte progenitor cells | 0 | 0.88 | Intergenic | LRRC43 | U |
| 4383 | chr7 | 5878899 | 5879036 | Erythrocyte progenitor cells | 0.02 | 0.9 | exon, intron | ZNF815P, ZNF815P | U |
| 4384 | chr6 | 161686743 | 161686906 | Erythrocyte progenitor cells | 0.01 | 0.89 | intron | AGPAT4 | U |
| 4385 | chr1 | 228333578 | 228333910 | Erythrocyte progenitor cells | 0.01 | 0.89 | intron | GUK1 | U |
| 4386 | chr2 | 1493006553 | 149300999 | Erythrocyte progenitor cells | 0 | 0.88 | Intergenic | EPC2 | U |
| 4387 | chr17 | 30626833 | 30627225 | Erythrocyte progenitor cells | 0.03 | 0.91 | intron | RHBDL3 | U |
| 4388 | chr3 | 49510811 | 49510850 | Erythrocyte progenitor cells | 0.04 | 0.91 | intron | DAG1 | U |
| 4389 | chr12 | 53704069 | 53704171 | Erythrocyte progenitor cells | 0.03 | 0.9 | intron | AAAS | U |
| 4390 | chr9 | 140283642 | 140283835 | Erythrocyte progenitor cells | 0.07 | 0.94 | intron | EXD3 | U |
| 4391 | chr1 | 179277528 | 179277802 | Erythrocyte progenitor cells | 0.07 | 0.94 | intron | SOAT1 | U |
| 4392 | chr2 | 74358977 | 74359290 | Erythrocyte progenitor cells | 0.04 | 0.91 | Intergenic | BOLA3 | U |
| 4393 | chr1 | 17044689 | 17044818 | Erythrocyte progenitor cells | 0.01 | 0.87 | intron | ESPNP | U |
| 4394 | chr2 | 2202188683 | 220219030 | Erythrocyte progenitor cells | 0.01 | 0.87 | Intergenic | RESP1B | U |
| 4395 | chr1 | 21680073 | 21680300 | Erythrocyte progenitor cells | 0.01 | 0.87 | Intergenic | LOC100506801 | U |
| 4396 | chr4 | 79731105 | 79731565 | Erythrocyte progenitor cells | 0.04 | 0.9 | intron | BMP2K | U |
| 4397 | chr17 | 81079169 | 81079664 | Erythrocyte progenitor cells | 0.06 | 0.91 | Intergenic | METRNL | U |
| 4398 | chr2 | 106002785 | 106002910 | Erythrocyte progenitor cells | 0.1 | 0.94 | exon | FHL2 | U |
| 4399 | chr19 | 42911473 | 42911641 | Erythrocyte progenitor cells | 0.06 | 0.9 | intron, exon, intron | LIPE, LIPE, LIPE | U |
| 4400 | chr14 | 101513595 | 101513839 | Erythrocyte progenitor cells | 0.02 | 0.86 | promoter-TSS | MIR539 | U |
| 4401 | chr7 | 5500335 | 5500580 | Erythrocyte progenitor cells | 0.09 | 0.93 | Intergenic | MIR589 | U |
| 4402 | chr3 | 186276568 | 186276814 | Erythrocyte progenitor cells | 0.11 | 0.94 | intron | TBCCD1 | U |
| 4403 | chr16 | 74546117 | 74546248 | Erythrocyte progenitor cells | 0.15 | 0.97 | intron | GLG1 | U |
| 4404 | chr3 | 156787192 | 156787465 | Erythrocyte progenitor cells | 0.08 | 0.9 | Intergenic | LOC100498859 | U |
| 4405 | chr8 | 145276559 | 145276678 | Erythrocyte progenitor cells | 0.08 | 0.89 | intron | MROH1 | U |
| 4406 | chr7 | 1990001 | 1990145 | Erythrocyte progenitor cells | 0.09 | 0.9 | intron | MADIL1 | U |
| 4407 | chr7 | 1894920 | 1895079 | Erythrocyte progenitor cells | 0.13 | 0.94 | intron | MAD1L1 | U |
| 4408 | chr16 | 577586103 | 57758778 | Erythrocyte progenitor cells | 0.08 | 0.89 | exon, intron | CCDC135, CCDC135 | U |
| 4409 | chr16 | 29666057 | 29666231 | Erythrocyte progenitor cells | 0.1 | 0.91 | Intergenic | SPN | U |
| 4410 | chr17 | 80419253 | 80419526 | Erythrocyte progenitor cells | 0.13 | 0.94 | intron | NARF | U |
| 4411 | chr5 | 1802090101 | 180209317 | Erythrocyte progenitor cells | 0.09 | 0.9 | Intergenic | MGAT1 | U |
| 4412 | chr10 | 127504782 | 127505166 | Erythrocyte progenitor cells | 0.14 | 0.95 | intron | UROS | U |
| 4413 | chr16 | 449076 | 449259 | Erythrocyte progenitor cells | 0.13 | 0.93 | exon, intron | NME4, NME4 | U |
| 4414 | chr12 | 53703422 | 53703823 | Erythrocyte progenitor cells | 0.05 | 0.85 | intron | AAAS | U |
| 4415 | chr3 | 197283517 | 197283945 | Erythrocyte progenitor cells | 0.12 | 0.92 | promoter-TSS | BDH1 | U |
| 4416 | chr2 | 9508904 | 9509139 | Erythrocyte progenitor cells | 0.13 | 0.92 | intron | ASAP2 | U |
| 4417 | chr11 | 65613991 | 65614226 | Erythrocyte progenitor cells | 0.08 | 0.87 | intron | SNX32 | U |
| 4418 | chr5 | 177604200 | 177604526 | Erythrocyte progenitor cells | 0.14 | 0.93 | Intergenic | GMCL1P1 | U |
| 4419 | chr13 | 114020135 | 114020574 | Erythrocyte progenitor cells | 0.12 | 0.91 | Intergenic | GRTP1 | U |
| 4420 | chr17 | 57779656 | 57780121 | Erythrocyte progenitor cells | 0.15 | 0.94 | intron | PTRH2 | U |
| 4421 | chr7 | 99892312 | 99892531 | Erythrocyte progenitor cells | 0.15 | 0.93 | Intergenic | SPDYE3 | U |
| 4422 | chr19 | 50376109 | 50376389 | Erythrocyte progenitor cells | 0.14 | 0.92 | intron, exon | AKT1S1, AKT1S1 | U |
| 4423 | chr7 | 66104515 | 66104804 | Erythrocyte progenitor cells | 0.13 | 0.91 | exon | KCTD7 | U |
| 4424 | chr17 | 2823992 | 2824289 | Erythrocyte progenitor cells | 0.14 | 0.92 | intron | RAP1GAP2 | U |
| 4425 | chr1 | 228113376 | 228113762 | Erythrocyte progenitor cells | 0.13 | 0.91 | intron | WNT9A | U |
| 4426 | chr3 | 45563583 | 45563981 | Erythrocyte progenitor cells | 0.15 | 0.93 | intron | LARS2 | U |
| 4427 | chr19 | 439487841 | 43948972 | Erythrocyte progenitor cells | 0.12 | 0.89 | Intergenic | LYPD3 | U |
| 4428 | chr15 | 78568978 | 78569357 | Erythrocyte progenitor cells | 0.07 | 0.84 | intron | DNAJA4 | U |
| 4429 | chr1 | 90363428 | 90363837 | Erythrocyte progenitor cells | 0.16 | 0.92 | intron | LRRC8D | U |
| 4430 | chr12 | 110533427 | 110533921 | Erythrocyte progenitor cells | 0.15 | 0.91 | Intergenic | IFT81 | U |
| 4431 | chr14 | 104197417 | 104197579 | Erythrocyte progenitor cells | 0.19 | 0.94 | intron | ZFYVE21 | U |
| 4432 | chr16 | 67615261 | 67615568 | Erythrocyte progenitor cells | 0.2 | 0.95 | intron | CTCF | U |
| 4433 | chr16 | 16248495 | 16248667 | Erythrocyte progenitor cells | 0.16 | 0.9 | exon | ABCC6 | U |
| 4434 | chr2 | 218931177 | 218931403 | Erythrocyte progenitor cells | 0.15 | 0.88 | intron | RUFY4 | U |
| 4435 | chr9 | 4850162 | 4850407 | Erythrocyte progenitor cells | 0.21 | 0.94 | promoter-TSS | MIR101-2 | U |
| 4436 | chr16 | 31029177 | 31029458 | Erythrocyte progenitor cells | 0.15 | 0.88 | Intergenic | STX18 | U |
| 4437 | chr21 | 46487394 | 46487786 | Erythrocyte progenitor cells | 0.16 | 0.89 | Intergenic | SSR4P1 | U |
| 4438 | chr3 | 127303699 | 127303878 | Erythrocyte progenitor cells | 0.22 | 0.94 | intron | TPRA1 | U |
| 4439 | chr19 | 2421753 | 2421967 | Erythrocyte progenitor cells | 0.21 | 0.93 | intron, exon | TMPRSS9, TMPRSS9 | U |
| 4440 | chr2 | 96837551 | 96837915 | Erythrocyte progenitor cells | 0.21 | 0.93 | Intergenic | DUSP2 | U |
| 4441 | chr19 | 12910879 | 12911053 | Erythrocyte progenitor cells | 0.18 | 0.89 | TTS | PRDX2 | U |
| 4442 | chr7 | 129593580 | 129593730 | Erythrocyte progenitor cells | 0.21 | 0.91 | promoter-TSS | UBE2H | U |
| 4443 | chr21 | 33940107 | 33940423 | Erythrocyte progenitor cells | 0.24 | 0.94 | Intergenic | TCP10L | U |
| 4444 | chr7 | 120648099 | 120648446 | Erythrocyte progenitor cells | 0.24 | 0.94 | intron | CPED1 | U |
| 4445 | chr12 | 1600710 | 1601104 | Erythrocyte progenitor cells | 0.22 | 0.92 | exor | ERC1 | U |
| 4446 | chr8 | 128904792 | 128905016 | Erythrocyte progenitor cells | 0.25 | 0.94 | intron | PVT1 | U |
| 4447 | chr1 | 19439018 | 19439348 | Erythrocyte progenitor cells | 0.26 | 0.95 | intron | UBR4 | U |
| 4448 | chr12 | 112170744 | 112171151 | Erythrocyte progenitor cells | 0.28 | 0.93 | intron | ACAD10 | U |
| 4449 | chr16 | 157905833 | 15790760 | Erythrocyte progenitor cells | 0.3 | 0.94 | intron | NDE1 | U |
| 4450 | chr16 | 68806081 | 68806566 | Erythrocyte progenitor cells | 0.03 | 0.91 | intron | CDH1 | U |
| 4451 | chr3 | 10178211 | 10178463 | Erythrocyte progenitor cells | 0.12 | 0.93 | Intergenic | VHL | U |
| 4452 | chr13 | 111948368 | 111948464 | Erythrocyte progenitor cells | 0.17 | 0.96 | TTS | ARHGEF7 | U |
| 4453 | chr14 | 103213119 | 103213348 | Erythrocyte progenitor cells | 0.17 | 0.91 | Intergenic | TRAF3 | U |
| 4454 | chr9 | 114911542 | 114911999 | Erythrocyte progenitor cells | 0.06 | 0.92 | intron | SUSD1 | U |
| 4455 | chr14 | 31666439 | 31666606 | Erythrocyte progenitor cells | 0 | 0.9 | intron | HECTD1 | U |
| 4456 | chr22 | 38532110 | 38532334 | Erythrocyte progenitor cells | 0.03 | 0.91 | intron | PLA2G6 | U |
| 4457 | chr9 | 1068642481 | 106864520 | Erythrocyte progenitor cells | 0.05 | 0.93 | intron, exon | SMC2, SMC2 | U |
| 4458 | chr19 | 5996750 | 5997048 | Erythrocyte progenitor cells | 0.01 | 0.89 | intron | RFX2 | U |
| 4459 | ofchi2 | 304954163 | 30495827 | Erythrocyte progenitor cells | 0.02 | 0.89 | intron | TTLL9 | U |
| 4460 | chr15 | 78555871 | 78556250 | Erythrocyte progenitor cells | 0.05 | 0.9 | promoter-TSS, Interg [symbol] | DNAJA4, DNAJA4 | U |
| 4461 | chr20 | 45993559 | 45993701 | Erythrocyte progenitor cells | 0.08 | 0.92 | Intergenic | ZMYND8 | U |
| 4462 | chr14 | 20871852 | 20872145 | Erythrocyte progenitor cells | 0.09 | 0.92 | intron | TEP1 | U |
| 4463 | chr14 | 39786036 | 39786529 | Erythrocyte progenitor cells | 0.16 | 0.95 | intron | CTAGE5 | U |
| 4464 | chr22 | 39884875 | 39885010 | Erythrocyte progenitor cells | 0.15 | 0.91 | exon | MGAT3 | U |
| 4465 | chr17 | 80171382 | 80171582 | Erythrocyte progenitor cells | 0.63 | 0.06 | promoter-TSS | CCDC57 | M |
| 4466 | chr6 | 157505409 | 157505527 | Erythrocyte progenitor cells | 0.55 | 0.11 | exon | ARID1B | M |
| 4467 | chr5 | 1274178341 | 127417987 | Erythrocyte progenitor cells | 0.58 | 0.21 | intron | FLI33630 | M |
| 4468 | chr11 | 796841 | 796992 | Erythrocyte progenitor cells | 0.57 | 0.22 | promoter-TSS | SLC25A22 | M |
| 4469 | chr1 | 151162943 | 151162973 | Erythrocyte progenitor cells | 0.54 | 0.21 | promoter-TSS | VPS72 | M |
| 4470 | chr8 | 89392239 | 89392496 | Erythrocyte progenitor cells | 0.82 | 0.87 | Intergenic | MMP16 | M |
| 4471 | chrX | 84117156 | 84117269 | Epidermal Keratinocytes | 0.03 | 0.92 | Intergenic | UBE2DNL | U |
| 4472 | chr5 | 2592741 | 2592950 | Epidermal Keratinocytes | 0.02 | 0.9 | Intergenic | IRX2 | U |
| 4473 | chr16 | 79143557 | 79143897 | Epidermal Keratinocytes | 0.03 | 0.9 | intron | WWOX | U |
| 4474 | chr3 | 65838606 | 65838836 | Epidermal Keratinocytes | 0.07 | 0.92 | intron | MAGI1 | U |
| 4475 | chr8 | 90867618 | 90867746 | Epidermal Keratinocytes | 0.11 | 0.95 | Intergenic | OSGIN2 | U |
| 4476 | chr12 | 4424217 | 4424338 | Epidermal Keratinocytes | 0.09 | 0.92 | Intergenic | C12orf5 | U |
| 4477 | chr12 | 106289786 | 106290035 | Epidermal Keratinocytes | 0.1 | 0.91 | Intergenic | NUAK1 | U |
| 4478 | chr19 | 2230349 | 2230507 | Epidermal Keratinocytes | 0.13 | 0.93 | exon | DOT1L | U |
| 4479 | chr1 | 32466777 | 32467150 | Epidermal Keratinocytes | 0.11 | 0.89 | Intergenic | KHDRBS1 | U |
| 4480 | chr21 | 46169969 | 46170228 | Epidermal Keratinocytes | 0.15 | 0.92 | Intergenic | TSPEAR | U |
| 4481 | chr1 | 6010708 | 6010977 | Epidermal Keratinocytes | 0.14 | 0.91 | intron | NPHP4 | U |
| 4482 | chr1 | 116471365 | 116471755 | Epidermal Keratinocytes | 0.15 | 0.91 | Intergenic | SLC22A15 | U |
| 4483 | chr15 | 35320312 | 35320433 | Epidermal Keratinocytes | 0.17 | 0.92 | Intergenic | ZNF770 | U |
| 4484 | chr3 | 37890977 | 37891333 | Epidermal Keratinocytes | 0.2 | 0.93 | Intergenic | CTDSPL | U |
| 4485 | chr12 | 106749774 | 106750248 | Epidermal Keratinocytes | 0.2 | 0.92 | Intergenic | POLR3B | U |
| 4486 | chr13 | 27699652 | 27700119 | Epidermal Keratinocytes | 0.27 | 0.96 | intron | USP12 | U |
| 4487 | chr12 | 6475922 | 6476358 | Epidermal Keratinocytes | 0.24 | 0.92 | intron | SCNN1A | U |
| 4488 | chr1 | 246959768 | 246960053 | Epidermal Keratinocytes | 0.27 | 0.94 | Intergenic | LOC149134 | U |
| 4489 | chr19 | 877313 | 877799 | Epidermal Keratinocytes | 0.27 | 0.93 | intron | MED16 | U |
| 4490 | chr11 | 61917560 | 61917804 | Epidermal Keratinocytes | 0.3 | 0.93 | intron | INCENP | U |
| 4491 | chr8 | 2135497 | 2135629 | Epidermal Keratinocytes | 0.03 | 0.63 | Intergenic | MYOM2 | U |
| 4492 | chr17 | 2172445 | 2172644 | Epidermal Keratinocytes | 0.35 | 0.95 | intron | SMG6 | U |
| 4493 | chr18 | 28876586 | 28876648 | Epidermal Keratinocytes | 0.05 | 0.93 | Intergenic | DSG1 | U |
| 4494 | chr15 | 52531305 | 52531368 | Epidermal Keratinocytes | 0.04 | 0.92 | intron | MYO5C | U |
| 4495 | chr19 | 48925153 | 48925209 | Epidermal Keratinocytes | 0.06 | 0.93 | exon, intron | GRIN2D, GRIN2D | U |
| 4496 | chr6 | 169850857 | 169850988 | Epidermal Keratinocytes | 0.03 | 0.9 | Intergenic | THBS2 | U |
| 4497 | chr11 | 76869347 | 76869413 | Epidermal Keratinocytes | 0.07 | 0.92 | intron | MYO7A | U |
| 4498 | chr15 | 28099623 | 28099842 | Epidermal Keratinocytes | 0.04 | 0.89 | intron | OCA2 | U |
| 4499 | chrX | 152998053 | 152998156 | Epidermal Keratinocytes | 0.08 | 0.92 | intron | ABCD1 | U |
| 4500 | chr11 | 48080498 | 48080643 | Epidermal Keratinocytes | 0.03 | 0.87 | intron | PTPRJ | U |
| 4501 | chr11 | 1140895363 | 114089817 | Epidermal Keratinocytes | 0.07 | 0.9 | intron | ZBTB16 | U |
| 4502 | chr1 | 246959348 | 246959700 | Epidermal Keratinocytes | 0.07 | 0.9 | Intergenic | LOC149134 | U |
| 4503 | chr12 | 92677309 | 92677664 | Epidermal Keratinocytes | 0.09 | 0.91 | Intergenic | BTG1 | U |
| 4504 | chr16 | 88760182 | 88760278 | Epidermal Keratinocytes | 0.1 | 0.91 | Intergenic | SNAI3 | U |
| 4505 | chr16 | 578492401 | 57849356 | Epidermal Keratinocytes | 0.06 | 0.87 | Intergenic | KIFC3 | U |
| 4506 | chr14 | 101868213 | 101868345 | Epidermal Keratinocytes | 0.07 | 0.88 | Intergenic | DIO3OS | U |
| 4507 | chrX | 152079723 | 152079875 | Epidermal Keratinocytes | 0.08 | 0.89 | Intergenic | ZNF185 | U |
| 4508 | chrX | 48349412 | 48349483 | Epidermal Keratinocytes | 0 | 0.8 | Intergenic | FTSJ1 | U |
| 4509 | chr9 | 136527053 | 136527191 | Epidermal Keratinocytes | 0.1 | 0.9 | Intergenic | DBH | U |
| 4510 | chr8 | 20176552 | 20176780 | Epidermal Keratinocytes | 0.12 | 0.92 | Intergenic | LZTS1-AS1 | U |
| 4511 | chr13 | 106891181 | 106891238 | Epidermal Keratinocytes | 0.11 | 0.9 | Intergenic | LINC00460 | U |
| 4512 | chr16 | 15803146 | 15803424 | Epidermal Keratinocytes | 0.14 | 0.93 | intron | MYH11 | U |
| 4513 | chr15 | 99659291 | 99659469 | Epidermal Keratinocytes | 0.1 | 0.88 | exon | SYNM | U |
| 4514 | chr1 | 32171523 | 32171741 | Epidermal Keratinocytes | 0.12 | 0.9 | Intergenic | COL16A1 | U |
| 4515 | chr8 | 2135178 | 2135470 | Epidermal Keratinocytes | 0.05 | 0.83 | Intergenic | MYOM2 | U |
| 4516 | chr10 | 91252311 | 91252644 | Epidermal Keratinocytes | 0.12 | 0.89 | intron | SLC16A12 | U |
| 4517 | chr6 | 82008699 | 82009196 | Epidermal Keratinocytes | 0.11 | 0.88 | Intergenic | FAM46A | U |
| 4518 | chr1 | 9390401 | 939130 | Epidermal Keratinocytes | 0.16 | 0.92 | Intergenic | HES4 | U |
| 4519 | chr1 | 385077801 | 38507887 | Epidermal Keratinocytes | 0.16 | 0.92 | Intergenic | POU3F1 | U |
| 4520 | chr1 | 116690861 | 116691020 | Epidermal Keratinocytes | 0.14 | 0.9 | Intergenic | MAB21L3 | U |
| 4521 | chr2 | 110424988 | 110425335 | Epidermal Keratinocytes | 0.13 | 0.89 | Intergenic | SOWAHC | U |
| 4522 | chr174 | 76553240 | 76553636 | Epidermal Keratinocytes | 0.09 | 0.85 | intron | DNAH17 | U |
| 4523 | chr14 | 98414975 | 98415030 | Epidermal Keratinocytes | 0.16 | 0.91 | intron | C14orf64 | U |
| 4524 | chr10 | 125117714 | 125117858 | Epidermal Keratinocytes | 0.1 | 0.85 | Intergenic | BUB3 | U |
| 4525 | chr5 | 80982809 | 80983081 | Epidermal Keratinocytes | 0.2 | 0.95 | intron | SSBP2 | U |
| 4526 | chr17 | 54926110 | 54926430 | Epidermal Keratinocytes | 0.18 | 0.93 | intron | DGKE | U |
| 4527 | chr9 | 1309387141 | 130939120 | Epidermal Keratinocytes | 0.19 | 0.94 | intron | CIZ1 | U |
| 4528 | chr7 | 2160533 | 2160617 | Epidermal Keratinocytes | 0.15 | 0.89 | intron | MAD1L1 | U |
| 4529 | chr10 | 44702307 | 44702403 | Epidermal Keratinocytes | 0.14 | 0.88 | Intergenic | LOC100130539 | U |
| 4530 | chr7 | 116085373 | 116085530 | Epidermal Keratinocytes | 0.15 | 0.89 | Intergenic | CAV2 | U |
| 4531 | chr1 | 214721551 | 214721786 | Epidermal Keratinocytes | 0.15 | 0.89 | intron | PTPN14 | U |
| 4532 | chr10 | 63424439 | 63424719 | Epidermal Keratinocytes | 0.14 | 0.88 | intron | C10orf107 | U |
| 4533 | chr1 | 2117017851 | 211702236 | Epidermal Keratinocytes | 0.11 | 0.85 | Intergenic | RD3 | U |
| 4534 | chrx | 49127163 | 49127402 | Epidermal Keratinocytes | 0.12 | 0.85 | promoter-TSS | PPP1R3F | U |
| 4535 | chr8 | 98391448 | 98391550 | Epidermal Keratinocytes | 0.16 | 0.88 | Intergenic | TSPYL5 | U |
| 4536 | chrX | 48330441 | 48330603 | Epidermal Keratinocytes | 0.11 | 0.83 | Intergenic | SLC38A5 | U |
| 4537 | chr7 | 152289520 | 152289720 | Epidermal Keratinocytes | 0.15 | 0.87 | Intergenic | XRCC2 | U |
| 4538 | chr17 | 48895912 | 48896138 | Epidermal Keratinocytes | 0.2 | 0.92 | Intergenic | WFIKKN2 | U |
| 4539 | chr21 | 44578204 | 44578445 | Epidermal Keratinocytes | 0.13 | 0.84 | Intergenic | CRYAA | U |
| 4540 | chr7 | 148180198 | 148180560 | Epidermal Keratinocytes | 0.22 | 0.93 | Intergenic | C7orf33 | U |
| 4541 | chr9 | 1329058163 | 132905902 | Epidermal Keratinocytes | 0.12 | 0.82 | Intergenic | NCS1 | U |
| 4542 | chr15 | 85372719 | 85372852 | Epidermal Keratinocytes | 0.22 | 0.92 | intron | ALPK3 | U |
| 4543 | chr9 | 139151659 | 139151843 | Epidermal Keratinocytes | 0.13 | 0.83 | Intergenic | QSOX2 | U |
| 4544 | chrX | 151807905 | 151808145 | Epidermal Keratinocytes | 0.11 | 0.81 | intron | GABRQ | U |
| 4545 | chr18 | 77938790 | 77938892 | Epidermal Keratinocytes | 0.2 | 0.89 | intron | PARD6G | U |
| 4546 | chr11 | 57126582 | 57126711 | Epidermal Keratinocytes | 0.15 | 0.84 | intron | P2RX3 | U |
| 4547 | chr17 | 71097077 | 71097238 | Epidermal Keratinocytes | 0.26 | 0.95 | Intergenic | SLC39A11 | U |
| 4548 | chr1 | 237546768 | 237547021 | Epidermal Keratinocytes | 0.19 | 0.88 | intron | RYR2 | U |
| 4549 | chr7 | 40938603 | 40938875 | Epidermal Keratinocytes | 0.15 | 0.84 | Intergenic | C7orf10 | U |
| 4550 | chr5 | 168202191 | 16820638 | Epidermal Keratinocytes | 0.23 | 0.92 | intron | MYO10 | U |
| 4551 | chr22 | 486052761 | 48605314 | Epidermal Keratinocytes | 0.16 | 0.84 | Intergenic | MIR3201 | U |
| 4552 | chr7 | 126298920 | 126299005 | Epidermal Keratinocytes | 0.2 | 0.88 | Intron | GRM8 | U |
| 4553 | chr17 | 639372 | 639583 | Epidermal Keratinocytes | 0.26 | 0.94 | intron | FAM57A | U |
| 4554 | chr16 | 54244135 | 54244396 | Epidermal Keratinocytes | 0.18 | 0.86 | Intergenic | IRX3 | U |
| 4555 | chr10 | 99239715 | 99240020 | Epidermal Keratinocytes | 0.19 | 0.87 | intron | MMS19 | U |
| 4556 | chr8 | 119044608 | 119044938 | Epidermal Keratinocytes | 0.23 | 0.91 | intron | EXT1 | U |
| 4557 | chr1 | 245786057 | 245786216 | Epidermal Keratinocytes | 0.24 | 0.91 | intron | KIF26B | U |
| 4558 | chr1 | 2055734691 | 205573736 | Epidermal Keratinocytes | 0.26 | 0.93 | Intergenic | ELK4 | U |
| 4559 | chr11 | 125276308 | 125276504 | Epidermal Keratinocytes | 0.2 | 0.86 | intron | PKNOX2 | U |
| 4560 | chr17 | 724151281 | 72415595 | Epidermal Keratinocytes | 0.24 | 0.9 | Intergenic | GPRC5C | U |
| 4561 | chr20 | 60306851 | 60307049 | Epidermal Keratinocytes | 0.18 | 0.82 | intron | CDH4 | U |
| 4562 | chr1 | 53995480 | 53995661 | Epidermal Keratinocytes | 0.27 | 0.9 | exon | GLIS1 | U |
| 4563 | chr16 | 23495499 | 23495664 | Epidermal Keratinocytes | 0.31 | 0.93 | intron | GGA2 | U |
| 4564 | chr19 | 46787721 | 46788042 | Epidermal Keratinocytes | 0.3 | 0.92 | intron | RNU6-66 | U |
| 4565 | chr15 | 50945080 | 50945477 | Epidermal Keratinocytes | 0.3 | 0.92 | intron | TRPM7 | U |
| 4566 | chr12 | 6475764 | 6475891 | Epidermal Keratinocytes | 0.31 | 0.92 | intron | SCNN1A | U |
| 4567 | chr17 | 62938635 | 62938768 | Epidermal Keratinocytes | 0.31 | 0.92 | Intergenic | LRRC37A3 | U |
| 4568 | chr13 | 24152430 | 24152564 | Epidermal Keratinocytes | 0.27 | 0.88 | intron | TNFRSF19 | U |
| 4569 | chr21 | 40128349 | 40128578 | Epidermal Keratinocytes | 0.34 | 0.93 | intron | LINC00114 | U |
| 4570 | chr1 | 236266130 | 236266146 | Epidermal Keratinocytes | 0.3 | 0.88 | Intergenic | NID1 | U |
| 4571 | chr17 | 79638116 | 79638334 | Epidermal Keratinocytes | 0.39 | 0.96 | intron | CCDC137 | U |
| 4572 | chr12 | 699039 | 699443 | Epidermal Keratinocytes | 0.36 | 0.92 | intron | NINJ2 | U |
| 4573 | chr11 | 62680073 | 6268253 | Epidermal Keratinocytes | 0.39 | 0.91 | Intergenic | CNGA4 | U |
| 4574 | chr10 | 8302733 | 8302841 | Epidermal Keratinocytes | 0.08 | 0.92 | Intergenic | GATA3 | U |
| 4575 | chrX | 1530573881 | 153057520 | Epidermal Keratinocytes | 0.13 | 0.88 | intron | IDH3G | U |
| 4576 | chr6 | 71602829 | 71603020 | Epidermal Keratinocytes | 0.14 | 0.87 | intron | B3GAT2 | U |
| 4577 | chr20 | 50884577 | 50884769 | Epidermal Keratinocytes | 0.21 | 0.91 | Intergenic | ZFP64 | U |
| 4578 | chr8 | 71185517 | 71185641 | Epidermal Keratinocytes | 0.05 | 0.92 | intron | NCOA2 | U |
| 4579 | chr1 | 236044087 | 236044286 | Epidermal Keratinocytes | 0.18 | 0.92 | Intergenic | LYST | U |
| 4580 | chr16 | 1017921 | 1018068 | Epidermal Keratinocytes | 0.07 | 0.92 | intron | LMF1 | U |
| 4581 | chr2 | 9960715 | 9960843 | Epidermal Keratinocytes | 0.05 | 0.87 | Intergenic | TAF1B | U |
| 4582 | chr6 | 12185473 | 12185769 | Epidermal Keratinocytes | 0.09 | 0.9 | Intergenic | EDN1 | U |
| 4583 | chr8 | 144203483 | 144203628 | Epidermal Keratinocytes | 0.12 | 0.91 | Intergenic | LY6H | U |
| 4584 | chr4 | 122144251 | 122144586 | Epidermal Keratinocytes | 0.11 | 0.89 | intron | TNIP3 | U |
| 4585 | chr9 | 34659141 | 34659216 | Epidermal Keratinocytes | 0.15 | 0.92 | intron | IL11RA | U |
| 4586 | chr7 | 54866720 | 54866924 | Epidermal Keratinocytes | 0.13 | 0.88 | Intergenic | SEC61G | U |
| 4587 | chr21 | 46169568 | 46169950 | Epidermal Keratinocytes | 0.13 | 0.88 | Intergenic | TSPEAR | U |
| 4588 | chr6 | 10115299 | 10115347 | Epidermal Keratinocytes | 0.21 | 0.93 | Intergenic | LOC100130275 | U |
| 4589 | chr14 | 24241129 | 24241240 | Epidermal Keratinocytes | 0.25 | 0.94 | Intergenic | DHRS2 | U |
| 4590 | chr8 | 144203694 | 144203777 | Epidermal Keratinocytes | 0.22 | 0.9 | Intergenic | LY6H | U |
| 4591 | chr11 | 60515803 | 60515948 | Epidermal Keratinocytes | 0.23 | 0.91 | Intergenic | MS4A15 | U |
| 4592 | chr16 | 88709751 | 88710019 | Epidermal Keratinocytes | 0.22 | 0.9 | TTS, exon | CYBA, CYBA | U |
| 4593 | chr17 | 60739758 | 60740225 | Epidermal Keratinocytes | 0.28 | 0.9 | intron | MRC2 | U |
| 4594 | chr1 | 2522399 | 2522847 | Epidermal Keratinocytes | 0.34 | 0.93 | exon | FAM213B | U |
| 4595 | chr3 | 179182829 | 179183167 | Epidermal Keratinocytes | 0.35 | 0.92 | Intergenic | GNB4 | U |
| 4596 | chr17 | 18345569 | 18345653 | Epidermal Keratinocytes | 0.96 | 0.11 | exon, intron | KRT16P1, KRT16P1 | M |
| 4597 | chr12 | 54384175 | 54384440 | Epidermal Keratinocytes | 0.69 | 0.12 | TTS | HOXC10 | M |
| 4598 | chrX | 152950260 | 152950499 | Epidermal Keratinocytes | 0.9 | 0.37 | Intergenic | SLC6A8 | M |
| 4599 | chr7 | 27225772 | 27225898 | Epidermal Keratinocytes | 0.86 | 0.18 | promoter-TSS | HOXA11 | M |
| 4600 | chr10 | 119294251 | 119294603 | Epidermal Keratinocytes | 0.88 | 0.09 | intron | EMX2OS | M |
| 4601 | chr19 | 52223109 | 52223208 | Epidermal Keratinocytes | 0.9 | 0.12 | intron | HAS1 | M |
| 4602 | chr5 | 3602865 | 3603307 | Epidermal Keratinocytes | 0.9 | 0.12 | Intergenic | IRX1 | M |
| 4603 | chr12 | 54360064 | 54360424 | Epidermal Keratinocytes | 0.92 | 0.15 | intron | HOTAIR | M |
| 4604 | chr12 | 54345855 | 54346116 | Epidermal Keratinocytes | 0.83 | 0.11 | Intergenic | HOXC12 | M |
| 4605 | chr1 | 209849084 | 209849396 | Epidermal Keratinocytes | 0.75 | 0.03 | exon | G0S2 | M |
| 4606 | chr5 | 134879183 | 134879241 | Epidermal Keratinocytes | 0.87 | 0.16 | Intergenic | NEUROG1 | M |
| 4607 | chr17 | 48049829 | 48049928 | Epidermal Keratinocytes | 0.77 | 0.07 | promoter-TSS | DLX4 | M |
| 4608 | chr12 | 54354783 | 54355008 | Epidermal Keratinocytes | 0.79 | 0.1 | Intergenic | HOXC12 | M |
| 4609 | chr12 | 54355009 | 54355431 | Epidermal Keratinocytes | 0.76 | 0.07 | TTS | HOTAIR | M |
| 4610 | chr12 | 54346155 | 54346439 | Epidermal Keratinocytes | 0.83 | 0.15 | Intergenic | HOXC12 | M |
| 4611 | chr17 | 48041265 | 48041556 | Epidermal Keratinocytes | 0.73 | 0.07 | Intergenic | DLX4 | M |
| 4612 | chr17 | 48041956 | 48042292 | Epidermal Keratinocytes | 0.8 | 0.14 | Intergenic | DLX4 | M |
| 4613 | chr7 | 27225396 | 27225734 | Epidermal Keratinocytes | 0.75 | 0.12 | promoter-TSS | HOXA11 | M |
| 4614 | chr2 | 219647056 | 219647233 | Epidermal Keratinocytes | 0.67 | 0.06 | exon, intron | CYP27A1, CYP27A1 | M |
| 4615 | chr12 | 54345258 | 54345650 | Epidermal Keratinocytes | 0.68 | 0.11 | Intergenic | HOXC12 | M |
| 4616 | chr12 | 54345660 | 54345841 | Epidermal Keratinocytes | 0.58 | 0.06 | Intergenic | HOXC12 | M |
| 4617 | chr5 | 78281663 | 78281984 | Epidermal Keratinocytes | 0.58 | 0.06 | promoter-TSS | ARSB | M |
| 4618 | chr12 | 54383396 | 54383766 | Epidermal Keratinocytes | 0.57 | 0.12 | exon | HOXC10 | M |
| 4619 | chr12 | 47298922 | 47298957 | Dermal Fibroblasts | 0.08 | 0.92 | Intergenic | SLC38A4 | U |
| 4620 | chr11 | 104454814 | 104454954 | Dermal Fibroblasts | 0.07 | 0.91 | Intergenic | CASP12 | U |
| 4621 | chr15 | 50684234 | 50684377 | Dermal Fibroblasts | 0.07 | 0.9 | Intergenic | MIR4712 | U |
| 4622 | chr13 | 68483269 | 68483339 | Dermal Fibroblasts | 0.05 | 0.88 | Intergenic | PCDH9 | U |
| 4623 | chr7 | 36650421 | 36650719 | Dermal Fibroblasts | 0.09 | 0.91 | intron | AOAH | U |
| 4624 | chr3 | 169250875 | 169251263 | Dermal Fibroblasts | 0.05 | 0.87 | intron | MECOM | U |
| 4625 | chr12 | 51943598 | 51943720 | Dermal Fibroblasts | 0.11 | 0.89 | Intergenic | SCN8A | U |
| 4626 | chr2 | 99690665 | 99690793 | Dermal Fibroblasts | 0.15 | 0.91 | intron | TSGA10 | U |
| 4627 | chr8 | 26238970 | 26239143 | Dermal Fibroblasts | 0.16 | 0.92 | Intergenic | BNIP3L | U |
| 4628 | chr8 | 13359161 | 13359514 | Dermal Fibroblasts | 0.09 | 0.85 | intron | DLC1 | U |
| 4629 | chr16 | 81183400 | 81183469 | Dermal Fibroblasts | 0.16 | 0.9 | exon | PKD1L2 | U |
| 4630 | chrX | 134033886 | 134034247 | Dermal Fibroblasts | 0.1 | 0.84 | intron | MOSPD1 | U |
| 4631 | chr17 | 6152148 | 6152348 | Dermal Fibroblasts | 0.18 | 0.91 | Intergenic | WSCD1 | U |
| 4632 | chr1 | 222080603 | 222080834 | Dermal Fibroblasts | 0.2 | 0.9 | Intergenic | DUSP10 | U |
| 4633 | chr19 | 36662820 | 36662960 | Dermal Fibroblasts | 0.07 | 0.76 | Intergenic | COX7A1 | U |
| 4634 | chr3 | 11791178 | 11791495 | Dermal Fibroblasts | 0.2 | 0.89 | Intergenic | VGLLA | U |
| 4635 | chr7 | 6266433 | 6266755 | Dermal Fibroblasts | 0.22 | 0.89 | intron | CYTH3 | U |
| 4636 | chr11 | 66816067 | 66816270 | Dermal Fibroblasts | 0.3 | 0.93 | exon | SYT12 | U |
| 4637 | chrY | 7287132 | 7287207 | Dermal Fibroblasts | 0.06 | 0.66 | Intergenic | PRKY | U |
| 4638 | chr17 | 64077760 | 64078210 | Dermal Fibroblasts | 0.35 | 0.95 | intron | CEP112 | U |
| 4639 | chr6 | 80776401 | 80776577 | Dermal Fibroblasts | 0.35 | 0.92 | Intergenic | BCKDHB | U |
| 4640 | chr7 | 65668495 | 65668767 | Dermal Fibroblasts | 0.32 | 0.88 | Intergenic | TPST1 | U |
| 4641 | chr17 | 79390776 | 79391088 | Dermal Fibroblasts | 0.09 | 0.61 | intron | BAHCC1 | U |
| 4642 | chr2 | 60457415 | 60457705 | Dermal Fibroblasts | 0.04 | 0.9 | Intergenic | MIR4432 | U |
| 4643 | chr18 | 7129948 | 7130102 | Dermal Fibroblasts | 0.09 | 0.85 | Intergenic | LAMA1 | U |
| 4644 | chr8 | 62322984 | 62323287 | Dermal Fibroblasts | 0.08 | 0.84 | intron | CLVS1 | U |
| 4645 | chr3 | 61649226 | 61649611 | Dermal Fibroblasts | 0.16 | 0.9 | intron | PTPRG | U |
| 4646 | chr16 | 81183402 | 81183470 | Dermal Fibroblasts | 0.17 | 0.9 | exon | PKD1L2 | U |
| 4647 | chr7 | 1182309 | 1182436 | Dermal Fibroblasts | 0.13 | 0.86 | Intergenic | C7orf50 | U |
| 4648 | chr5 | 4023345 | 4023729 | Dermal Fibroblasts | 0.1 | 0.83 | Intergenic | IRX1 | U |
| 4649 | chr13 | 114620058 | 114620176 | Dermal Fibroblasts | 0.13 | 0.85 | Intergenic | LINC00565 | U |
| 4650 | chr10 | 2409461 | 2409680 | Dermal Fibroblasts | 0.16 | 0.88 | Intergenic | LINC00701 | U |
| 4651 | chr17 | 54337761 | 54338003 | Dermal Fibroblasts | 0.1 | 0.82 | intron | ANKFN1 | U |
| 4652 | chr13 | 22330275 | 22330375 | Dermal Fibroblasts | 0.15 | 0.86 | Intergenic | FGF9 | U |
| 4653 | chr2 | 1113111 | 1113451 | Dermal Fibroblasts | 0.09 | 0.8 | intron | SNTG2 | U |
| 4654 | chr10 | 102566154 | 102566256 | Dermal Fibroblasts | 0.16 | 0.86 | exon | PAX2 | U |
| 4655 | chr22 | 49061263 | 49061300 | Dermal Fibroblasts | 0.18 | 0.87 | intron | FAM19A5 | U |
| 4656 | chr6 | 167384565 | 167384733 | Dermal Fibroblasts | 0.21 | 0.9 | Intergenic | RNASET2 | U |
| 4657 | chr10 | 110085565 | 110085777 | Dermal Fibroblasts | 0.18 | 0.87 | Intergenic | RNU6-53 | U |
| 4658 | chr11 | 1639388 | 1639550 | Dermal Fibroblasts | 0.2 | 0.88 | intron | MOB2 | U |
| 4659 | chr2 | 28575383 | 28575616 | Dermal Fibroblasts | 0.2 | 0.88 | Intergenic | FOSL2 | U |
| 4660 | chr18 | 21300187 | 21300466 | Dermal Fibroblasts | 0.21 | 0.89 | intron | LAMA3 | U |
| 4661 | chr7 | 155211140 | 155211198 | Dermal Fibroblasts | 0.21 | 0.88 | Intergenic | EN2 | U |
| 4662 | chr21 | 40021088 | 40021155 | Dermal Fibroblasts | 0.24 | 0.91 | Intron | ERG | U |
| 4663 | chr6 | 1406547 | 1406727 | Dermal Fibroblasts | 0.15 | 0.82 | Intergenic | FOXF2 | U |
| 4664 | chr2 | 62443595 | 62443884 | Dermal Fibroblasts | 0.2 | 0.87 | intron | B3GNT2 | U |
| 4665 | chr13 | 23820643 | 23820940 | Dermal Fibroblasts | 0.2 | 0.87 | intron | SGCG | U |
| 4666 | chr18 | 26042946 | 26043292 | Dermal Fibroblasts | 0.19 | 0.86 | Intergenic | CDH2 | U |
| 4667 | chr17 | 7256414 | 7256482 | Dermal Fibroblasts | 0.19 | 0.85 | exon | KCTD11 | U |
| 4668 | chr4 | 183312528 | 183312683 | Dermal Fibroblasts | 0.19 | 0.85 | intron | TENM3 | U |
| 4669 | chr15 | 99981501 | 99981772 | Dermal Fibroblasts | 0.22 | 0.88 | Intergenic | MEF2A | U |
| 4670 | chrX | 25734836 | 25735304 | Dermal Fibroblasts | 0.12 | 0.78 | Intergenic | MAGEB18 | U |
| 4671 | chr15 | 89111807 | 89112040 | Dermal Fibroblasts | 0.16 | 0.81 | Intergenic | DET1 | U |
| 4672 | chr17 | 54512024 | 54512329 | Dermal Fibroblasts | 0.21 | 0.86 | intron | ANKFN1 | U |
| 4673 | chr5 | 91244079 | 91244449 | Dermal Fibroblasts | 0.19 | 0.84 | Intergenic | ARRDC3 | U |
| 4674 | chr5 | 120422725 | 120422805 | Dermal Fibroblasts | 0.22 | 0.86 | Intergenic | PRR16 | U |
| 4675 | chr5 | 898191 | 89958 | Dermal Fibroblasts | 0.23 | 0.87 | Intergenic | PLEKHG4B | U |
| 4676 | chrX | 18852174 | 18852336 | Dermal Fibroblasts | 0.17 | 0.81 | Intergenic | PHKA2-AS1 | U |
| 4677 | chr14 | 81712529 | 81712902 | Dermal Fibroblasts | 0.23 | 0.87 | Intergenic | GTF2A1 | U |
| 4678 | chr17 | 15242034 | 15242125 | Dermal Fibroblasts | 0.31 | 0.94 | intron | TEKT3 | U |
| 4679 | chr13 | 114620235 | 114620337 | Dermal Fibroblasts | 0.24 | 0.87 | Intergenic | LINC00565 | U |
| 4680 | chr21 | 41218176 | 41218297 | Dermal Fibroblasts | 0.28 | 0.91 | Intergenic | PCP4 | U |
| 4681 | chr5 | 166774911 | 166775092 | Dermal Fibroblasts | 0.26 | 0.89 | intron | TENM2 | U |
| 4682 | chr18 | 52781109 | 52781340 | Dermal Fibroblasts | 0.25 | 0.88 | Intergenic | CCDC68 | U |
| 4683 | chrX | 118747545 | 118747628 | Dermal Fibroblasts | 0.22 | 0.84 | Intergenic | NKRF | U |
| 4684 | chr11 | 130886648 | 130887074 | Dermal Fibroblasts | 0.27 | 0.89 | Intergenic | SNX19 | U |
| 4685 | chr5 | 66934952 | 66935091 | Dermal Fibroblasts | 0.27 | 0.88 | Intergenic | CD180 | U |
| 4686 | chr2 | 145408181 | 145408618 | Dermal Fibroblasts | 0.29 | 0.9 | Intergenic | DKFZp68501327 | U |
| 4687 | chr8 | 95040092 | 95040575 | Dermal Fibroblasts | 0.28 | 0.89 | Intergenic | PDP1 | U |
| 4688 | chr11 | 42272254 | 42272345 | Dermal Fibroblasts | 0.27 | 0.87 | intron | LOC100507205 | U |
| 4689 | chr16 | 62994506 | 62994647 | Dermal Fibroblasts | 0.31 | 0.91 | Intergenic | CDH8 | U |
| 4690 | chr10 | 28685637 | 28685825 | Dermal Fibroblasts | 0.24 | 0.84 | Intergenic | MPP7 | U |
| 4691 | chr13 | 87215142 | 87215336 | Dermal Fibroblasts | 0.27 | 0.87 | Intergenic | SLITRK6 | U |
| 4692 | chr5 | 5132809 | 5133037 | Dermal Fibroblasts | 0.3 | 0.9 | Intergenic | ADAMTS16 | U |
| 4693 | chr11 | 72415227 | 72415280 | Dermal Fibroblasts | 0.29 | 0.88 | exon | ARAP1 | U |
| 4694 | chr14 | 101864895 | 101864968 | Dermal Fibroblasts | 0.32 | 0.91 | Intergenic | DIO3OS | U |
| 4695 | chr18 | 73888077 | 73888224 | Dermal Fibroblasts | 0.3 | 0.89 | Intergenic | LOC339298 | U |
| 4696 | chr2 | 165655267 | 165655525 | Dermal Fibroblasts | 0.34 | 0.93 | intron | COBLL1 | U |
| 4697 | chr8 | 41328179 | 41328507 | Dermal Fibroblasts | 0.2 | 0.79 | Intergenic | GOLGA7 | U |
| 4698 | chr16 | 68396507 | 68396843 | Dermal Fibroblasts | 0.26 | 0.85 | intron | SMPD3 | U |
| 4699 | chr15 | 89270476 | 89270532 | Dermal Fibroblasts | 0.29 | 0.87 | Intergenic | ACAN | U |
| 4700 | chr13 | 101717738 | 101717918 | Dermal Fibroblasts | 0.31 | 0.89 | exon | NALCN | U |
| 4701 | chr21 | 46248887 | 46249273 | Dermal Fibroblasts | 0.27 | 0.85 | Intergenic | SUMO3 | U |
| 4702 | chr1 | 87338081 | 87338253 | Dermal Fibroblasts | 0.37 | 0.94 | intron | SEP15 | U |
| 4703 | chr1 | 71240943 | 71241182 | Dermal Fibroblasts | 0.23 | 0.8 | Intergenic | ZRANB2-AS1 | U |
| 4704 | chr3 | 72628306 | 72628676 | Dermal Fibroblasts | 0.23 | 0.8 | Intergenic | RYBP | U |
| 4705 | chr19 | 6207787 | 6208210 | Dermal Fibroblasts | 0.27 | 0.84 | Intergenic | MLLT1 | U |
| 4706 | chr8 | 144457024 | 144457465 | Dermal Fibroblasts | 0.32 | 0.89 | intron | RHPN1 | U |
| 4707 | chr16 | 86901654 | 86901892 | Dermal Fibroblasts | 0.31 | 0.87 | Intergenic | FOXLI | U |
| 4708 | chr19 | 8085903 | 8086328 | Dermal Fibroblasts | 0.29 | 0.85 | Intergenic | ELAVL1 | U |
| 4709 | chrX | 3491869 | 3492347 | Dermal Fibroblasts | 0.26 | 0.82 | Intergenic | PRKX | U |
| 4710 | chr18 | 65647088 | 65647305 | Dermal Fibroblasts | 0.27 | 0.82 | Intergenic | DSEL | U |
| 4711 | chr6 | 166249801 | 166249862 | Dermal Fibroblasts | 0.36 | 0.9 | Intergenic | LINC00602 | U |
| 4712 | chr12 | 295430611 | 29543199 | Dermal Fibroblasts | 0.36 | 0.9 | intron | LOC101055625 | U |
| 4713 | chr10 | 766464 | 766765 | Dermal Fibroblasts | 0.36 | 0.9 | Intergenic | DIP2C | U |
| 4714 | chr3 | 21847797 | 21847989 | Dermal Fibroblasts | 0.35 | 0.88 | Intergenic | ZNF385D | U |
| 4715 | chr15 | 98648149 | 98648258 | Dermal Fibroblasts | 0.39 | 0.9 | Intergenic | ARRDC4 | U |
| 4716 | chr2 | 232739219 | 232739571 | Dermal Fibroblasts | 0.33 | 0.84 | Intergenic | MIR1471 | U |
| 4717 | chr5 | 62400816 | 62401078 | Dermal Fibroblasts | 0.4 | 0.9 | Intergenic | LRRC70 | U |
| 4718 | chr7 | 151097193 | 151097689 | Dermal Fibroblasts | 0.39 | 0.89 | intron | WDR86 | U |
| 4719 | chr2 | 1096982181 | 109698498 | Dermal Fibroblasts | 0.37 | 0.85 | Intergenic | SH3RF3 | U |
| 4720 | chr8 | 41118707 | 41119167 | Dermal Fibroblasts | 0.08 | 0.84 | TTS | SFRP1 | U |
| 4721 | chr13 | 1117705803 | 111770837 | Dermal Fibroblasts | 0.1 | 0.87 | intron | ARHGEF7 | U |
| 4722 | chr21 | 27843249 | 27843440 | Dermal Fibroblasts | 0.16 | 0.88 | intron | CYYR1 | U |
| 4723 | chr11 | 105695367 | 105695635 | Dermal Fibroblasts | 0.15 | 0.84 | intron | GRIA4 | U |
| 4724 | chr12 | 13966164 | 13966589 | Dermal Fibroblasts | 0.18 | 0.83 | Intron | GRIN2B | U |
| 4725 | chr21 | 34314271 | 34314436 | Dermal Fibroblasts | 0.28 | 0.89 | Intergenic | OLIG2 | U |
| 4726 | chr2 | 85289565 | 85289866 | Dermal Fibroblasts | 0.28 | 0.88 | Intergenic | TCF7L1 | U |
| 4727 | chr4 | 190748609 | 190748983 | Dermal Fibroblasts | 0.27 | 0.86 | Intergenic | FRG1 | U |
| 4728 | chr6 | 9564844 | 9564989 | Dermal Fibroblasts | 0.19 | 0.88 | Intergenic | LOC100130275 | U |
| 4729 | chr20 | 20913889 | 20914064 | Dermal Fibroblasts | 0.11 | 0.88 | Intergenic | PLK1S1 | U |
| 4730 | chr12 | 14819230 | 14819535 | Dermal Fibroblasts | 0.19 | 0.91 | intron | GUCY2C | U |
| 4731 | chr13 | 114075763 | 114075950 | Dermal Fibroblasts | 0.17 | 0.86 | TTS | ADPRHL1 | U |
| 4732 | chr9 | 77399441 | 77399749 | Dermal Fibroblasts | 0.15 | 0.83 | intron | TRPM6 | U |
| 4733 | chr11 | 64427940 | 64428279 | Dermal Fibroblasts | 0.17 | 0.84 | intron | NRXN2 | U |
| 4734 | chr22 | 23138766 | 23139149 | Dermal Fibroblasts | 0.24 | 0.89 | Intergenic | MIR650 | U |
| 4735 | chr16 | 84823111 | 84823267 | Dermal Fibroblasts | 0.29 | 0.88 | Intergenic | CRISPLD2 | U |
| 4736 | chr14 | 88527213 | 88527494 | Dermal Fibroblasts | 0.28 | 0.86 | intron | LOC283587 | U |
| 4737 | chr20 | 16542545 | 16542727 | Dermal Fibroblasts | 0.31 | 0.88 | intron | KIF16B | U |
| 4738 | chr5 | 177708296 | 177708499 | Dermal Fibroblasts | 0.34 | 0.89 | intron | COL23A1 | U |
| 4739 | chr8 | 136440032 | 136440406 | Dermal Fibroblasts | 0.36 | 0.89 | Intergenic | KHDRBS3 | U |
| 4740 | chr10 | 112326274 | 112326400 | Dermal Fibroblasts | 0.37 | 0.86 | Intergenic | SMC3 | U |
| 4741 | chr20 | 41965620 | 41965878 | Dermal Fibroblasts | 0.39 | 0.87 | Intergenic | SRSF6 | U |
| 4742 | chr7 | 27241913 | 27242098 | Dermal Fibroblasts | 0.85 | 0.12 | exon | HOTTIP | M |
| 4743 | chr7 | 27242911 | 27243256 | Dermal Fibroblasts | 0.89 | 0.17 | intron | HOTTIP | M |
| 4744 | chr2 | 1068862141 | 106886708 | Dermal Fibroblasts | 0.73 | 0.05 | Intergenic | UXS1 | M |
| 4745 | chr12 | 115130140 | 115130437 | Dermal Fibroblasts | 0.54 | 0.04 | Intergenic | TBX3 | M |
| 4746 | chr1 | 148604014 | 148604095 | Dermal Fibroblasts | 0.77 | 0.39 | Intergenic | PPIAL4E | M |
| 4747 | chr4 | 190991531 | 190991966 | Dermal Fibroblasts | 0.91 | 0.57 | promoter-TSS | DUX4L6 | M |
| 4748 | chr11 | 44333794 | 44334264 | Dermal Fibroblasts | 0.7 | 0.12 | Intergenic | ALX4 | M |
| 4749 | chr2 | 119592075 | 119592516 | Dermal Fibroblasts | 0.95 | 0.13 | Intergenic | EN1 | M |
| 4750 | chr3 | 157815256 | 157815624 | Dermal Fibroblasts | 0.89 | 0.08 | exon | SHOX2 | M |
| 4751 | chr2 | 119592543 | 119592929 | Dermal Fibroblasts | 0.84 | 0.06 | Intergenic | EN1 | M |
| 4752 | chr1 | 119535707 | 119535963 | Dermal Fibroblasts | 0.91 | 0.16 | Intergenic | TBX15 | M |
| 4753 | chr2 | 119614187 | 119614576 | Dermal Fibroblasts | 0.83 | 0.09 | Intergenic | EN1 | M |
| 4754 | chr3 | 157816222 | 157816601 | Dermal Fibroblasts | 0.84 | 0.11 | intron | SHOX2 | M |
| 4755 | chr7 | 27232857 | 27232967 | Dermal Fibroblasts | 0.8 | 0.1 | Intergenic | HOXA13 | M |
| 4756 | chr2 | 1752079433 | 175208160 | Dermal Fibroblasts | 0.85 | 0.15 | Intergenic | SP9 | M |
| 4757 | chr2 | 175195675 | 175196043 | Dermal Fibroblasts | 0.86 | 0.16 | TTS | LOC285084 | M |
| 4758 | chr2 | 175208488 | 175208650 | Dermal Fibroblasts | 0.81 | 0.12 | Intergenic | SP9 | M |
| 4759 | chr2 | 1196145941 | 119614858 | Dermal Fibroblasts | 0.76 | 0.07 | Intergenic | EN1 | M |
| 4760 | chr7 | 27233045 | 27233242 | Dermal Fibroblasts | 0.85 | 0.17 | Intergenic | HOXA13 | M |
| 4761 | chr7 | 27237063 | 27237340 | Dermal Fibroblasts | 0.82 | 0.16 | exon | HOXA13 | M |
| 4762 | chr2 | 175196199 | 175196562 | Dermal Fibroblasts | 0.81 | 0.15 | Intergenic | SP9 | M |
| 4763 | chr7 | 27240758 | 27241133 | Dermal Fibroblasts | 0.72 | 0.11 | exor | HOTTIP | M |
| 4764 | chr8 | 17271025 | 17271348 | Dermal Fibroblasts | 0.67 | 0.08 | promoter-TSS | MTMR7 | M |
| 4765 | chr2 | 119614872 | 119615072 | Dermal Fibroblasts | 0.67 | 0.1 | Intergenic | EN1 | M |
| 4766 | chr3 | 157812688 | 157812966 | Dermal Fibroblasts | 0.69 | 0.12 | TTS | SHOX2 | M |
| 4767 | chr13 | 76765436 | 76765494 | Osteoblasts | 0.01 | 0.89 | Intergenic | C13orf45 | U |
| 4768 | chr6 | 143447049 | 143447402 | Osteoblasts | 0.05 | 0.93 | intron | AIG1 | U |
| 4769 | chr7 | 6952692 | 6952930 | Osteoblasts | 0.06 | 0.92 | Intergenic | CCZ1B | U |
| 4770 | chr2 | 39646607 | 39647084 | Osteoblasts | 0.02 | 0.88 | intron | MAP4K3 | U |
| 4771 | chr1 | 244532629 | 244532893 | Osteoblasts | 0.07 | 0.91 | intron | C1orf100 | U |
| 4772 | chr19 | 57454683 | 57454949 | Osteoblasts | 0.08 | 0.92 | Intergenic | MIMT1 | U |
| 4773 | chr13 | 76765181 | 76765309 | Osteoblasts | 0.08 | 0.91 | Intergenic | C13orf45 | U |
| 4774 | chr1 | 118130149 | 118130486 | Osteoblasts | 0.09 | 0.92 | Intergenic | FAM46C | U |
| 4775 | chr17 | 206787531 | 20678921 | Osteoblasts | 0.11 | 0.93 | Intergenic | LOC100287072 | U |
| 4776 | chr4 | 7107002 | 7107094 | Osteoblasts | 0.12 | 0.91 | Intergenic | FLI36777 | U |
| 4777 | chr12 | 19589066 | 19589285 | Osteoblasts | 0.1 | 0.89 | Intergenic | AEBP2 | U |
| 4778 | chr16 | 86264260 | 86264308 | Osteoblasts | 0.12 | 0.89 | Intergenic | LOC146513 | U |
| 4779 | chr11 | 1520812 | 1520881 | Osteoblasts | 0.26 | 0.97 | intron | MOB2 | U |
| 4780 | chr5 | 1501806 | 1502130 | Osteoblasts | 0.24 | 0.95 | intron | LPCAT1 | U |
| 4781 | chr2 | 34887501 | 3489235 | Osteoblasts | 0.23 | 0.92 | Intergenic | ADI1 | U |
| 4782 | chr7 | 105159466 | 105159943 | Osteoblasts | 0.29 | 0.95 | intron | PUS7 | U |
| 4783 | chr5 | 154334374 | 154334648 | Osteoblasts | 0.35 | 0.96 | intron | MRPL22 | U |
| 4784 | chr10 | 100288820 | 100288831 | Osteoblasts | 0.04 | 0.96 | intron | HPSE2 | U |
| 4785 | chr15 | 89238600 | 89238630 | Osteoblasts | 0.03 | 0.91 | Intergenic | ISG20 | U |
| 4786 | chr8 | 117168001 | 117168155 | Osteoblasts | 0.03 | 0.91 | intron | LINC00536 | U |
| 4787 | chr4 | 184962449 | 184962691 | Osteoblasts | 0.04 | 0.91 | Intergenic | STOX2 | U |
| 4788 | chr1 | 172215845 | 172216338 | Osteoblasts | 0.03 | 0.9 | intron | DNM3 | U |
| 4789 | chr7 | 730057 | 730536 | Osteoblasts | 0.06 | 0.92 | intron | PRKAR1B | U |
| 4790 | chr10 | 3856157 | 3856583 | Osteoblasts | 0.02 | 0.87 | Intergenic | KLF6 | U |
| 4791 | chr17 | 77713422 | 77713480 | Osteoblasts | 0.04 | 0.88 | intron | ENPP7 | U |
| 4792 | chr10 | 100288470 | 100288791 | Osteoblasts | 0.01 | 0.85 | intron | HPSE2 | U |
| 4793 | chr17 | 40997798 | 40997852 | Osteoblasts | 0.03 | 0.86 | exon | AOC2 | U |
| 4794 | chr9 | 138428373 | 138428451 | Osteoblasts | 0.03 | 0.86 | Intergenic | OBP2A | U |
| 4795 | chr1 | 10881052 | 10881210 | Osteoblasts | 0.05 | 0.88 | Intergenic | CASZ1 | U |
| 4796 | chr4 | 16695064 | 16695222 | Osteoblasts | 0.05 | 0.88 | intron | LDB2 | U |
| 4797 | chr12 | 132431728 | 132431778 | Osteoblasts | 0.11 | 0.93 | Intergenic | EP400 | U |
| 4798 | chr2 | 237228919 | 237228993 | Osteoblasts | 0.05 | 0.87 | Intergenic | ASB18 | U |
| 4799 | chr7 | 71790979 | 71791077 | Osteoblasts | 0.07 | 0.89 | intron | CALNJ | U |
| 4800 | chr9 | 124206177 | 124206331 | Osteoblasts | 0.09 | 0.91 | Intergenic | GGTA1P | U |
| 4801 | chr17 | 12949850 | 12950004 | Osteoblasts | 0.06 | 0.88 | Intergenic | ELAC2 | U |
| 4802 | chr17 | 16833421 | 16833663 | Osteoblasts | 0.08 | 0.9 | Intergenic | TNFRSF13B | U |
| 4803 | chr3 | 182999060 | 182999530 | Osteoblasts | 0.06 | 0.88 | intron | MCF2L2 | U |
| 4804 | chr17 | 77713718 | 77713771 | Osteoblasts | 0.07 | 0.88 | intron | ENPP7 | U |
| 4805 | chr12 | 111522484 | 111522676 | Osteoblasts | 0.09 | 0.9 | intron | CUX2 | U |
| 4806 | chr6 | 169110971 | 169111170 | Osteoblasts | 0.06 | 0.87 | Intergenic | SMOC2 | U |
| 4807 | chr18 | 46486493 | 46486702 | Osteoblasts | 0.07 | 0.88 | Intergenic | SMAD7 | U |
| 4808 | chr6 | 107204712 | 107204932 | Osteoblasts | 0.09 | 0.9 | intron | LOC100422737 | U |
| 4809 | chr5 | 159566311 | 159566544 | Osteoblasts | 0.08 | 0.89 | Intergenic | PWWP2A | U |
| 4810 | chr10 | 28490796 | 28491051 | Osteoblasts | 0.06 | 0.87 | intron | MPP7 | U |
| 4811 | chr4 | 186835453 | 186835753 | Osteoblasts | 0.09 | 0.9 | intron | SORBS2 | U |
| 4812 | chr1 | 22729239 | 22729326 | Osteoblasts | 0.07 | 0.87 | Intergenic | ZBTB40 | U |
| 4813 | chr2 | 23531183 | 235311959 | Osteoblasts | 0.02 | 0.82 | Intergenic | ARL4C | U |
| 4814 | chr7 | 74254530 | 74254686 | Osteoblasts | 0.1 | 0.9 | intron | GTF2IRD2 | U |
| 4815 | chr9 | 125878907 | 125879080 | Osteoblasts | 0.13 | 0.93 | Intergenic | MIR600HG | U |
| 4816 | chr17 | 71334790 | 71334988 | Osteoblasts | 0.1 | 0.9 | exon | SDK2 | U |
| 4817 | chr17 | 70318036 | 70318269 | Osteoblasts | 0.08 | 0.88 | Intergenic | SOX9 | U |
| 4818 | chr4 | 27056084 | 27056412 | Osteoblasts | 0.09 | 0.89 | Intergenic | STIM2 | U |
| 4819 | chr4 | 188104455 | 188104695 | Osteoblasts | 0.08 | 0.87 | Intergenic | LOC339975 | U |
| 4820 | chr2 | 100214336 | 100214597 | Osteoblasts | 0.07 | 0.86 | intron | AFF3 | U |
| 4821 | chr2 | 237105887 | 237106294 | Osteoblasts | 0.07 | 0.86 | intron | ASB18 | U |
| 4822 | chr12 | 18868524 | 18868939 | Osteoblasts | 0.06 | 0.85 | intron | PLCZ1 | U |
| 4823 | chr5 | 141633552 | 141633688 | Osteoblasts | 0.08 | 0.86 | Intergenic | SPRY4 | U |
| 4824 | chr3 | 145899029 | 145899202 | Osteoblasts | 0.14 | 0.92 | Intergenic | PLOD2 | U |
| 4825 | chr2 | 47763919 | 47764260 | Osteoblasts | 0.12 | 0.9 | intron | KCNK12 | U |
| 4826 | chr15 | 85826295 | 85826642 | Osteoblasts | 0.11 | 0.89 | Intergenic | LOC642423 | U |
| 4827 | chr3 | 175598604 | 175599090 | Osteoblasts | 0.09 | 0.87 | Intergenic | MIR4789 | U |
| 4828 | chr5 | 175863425 | 175863560 | Osteoblasts | 0.09 | 0.86 | Intergenic | FAF2 | U |
| 4829 | chr15 | 65234718 | 65234984 | Osteoblasts | 0.09 | 0.86 | exon, intron | ANKDD1A, ANKDD1A | U |
| 4830 | chr11 | 46953537 | 46953836 | Osteoblasts | 0.11 | 0.88 | Intergenic | C11orf49 | U |
| 4831 | chr2 | 225464554 | 225464869 | Osteoblasts | 0.14 | 0.91 | Intergenic | CUL3 | U |
| 4832 | chr2 | 121222350 | 121222490 | Osteoblasts | 0.15 | 0.91 | exon | LOC84931 | U |
| 4833 | chr15 | 86557719 | 86557900 | Osteoblasts | 0.13 | 0.89 | Intergenic | AGBL1 | U |
| 4834 | chr17 | 59361606 | 59361958 | Osteoblasts | 0.17 | 0.93 | intron | BCAB3 | U |
| 4835 | chr1 | 245242718 | 245242841 | Osteoblasts | 0.06 | 0.81 | intron | EFCAB2 | U |
| 4836 | chr8 | 75009771 | 75009896 | Osteoblasts | 0.15 | 0.9 | Intergenic | LY96 | U |
| 4837 | chr2 | 175631591 | 175631770 | Osteoblasts | 0.12 | 0.87 | Intergenic | CHRNA1 | U |
| 4838 | chr14 | 101171821 | 101172072 | Osteoblasts | 0.15 | 0.9 | Intergenic | DLK1 | U |
| 4839 | chr15 | 77253399 | 77253650 | Osteoblasts | 0.12 | 0.87 | Intergenic | RCN2 | U |
| 4840 | chr12 | 2483309 | 2483755 | Osteoblasts | 0.09 | 0.84 | intron | CACNA1C | U |
| 4841 | chr5 | 39615979 | 39616082 | Osteoblasts | 0.18 | 0.92 | Intergenic | DAB2 | U |
| 4842 | chr21 | 39324736 | 39324840 | Osteoblasts | 0.1 | 0.84 | Intergenic | KCNJ6 | U |
| 4843 | chr6 | 157906764 | 157906994 | Osteoblasts | 0.16 | 0.89 | intron | ZDHHC14 | U |
| 4844 | chr2 | 172753519 | 172753976 | Osteoblasts | 0.17 | 0.9 | Intergenic | SLC25A12 | U |
| 4845 | chr2 | 205337267 | 205337491 | Osteoblasts | 0.13 | 0.85 | Intergenic | PARD3B | U |
| 4846 | chr5 | 153913670 | 153914092 | Osteoblasts | 0.13 | 0.85 | Intergenic | HAND1 | U |
| 4847 | chr8 | 123106884 | 123107335 | Osteoblasts | 0.16 | 0.88 | Intergenic | HAS2 | U |
| 4848 | chr4 | 53536853 | 53537331 | Osteoblasts | 0.13 | 0.85 | Intergenic | USP46 | U |
| 4849 | chr10 | 74673021 | 74673364 | Osteoblasts | 0.16 | 0.86 | exon, intron | OIT3, OIT3 | U |
| 4850 | chr6 | 1179370311 | 117937449 | Osteoblasts | 0.2 | 0.9 | Intergenic | GOPC | U |
| 4851 | chr22 | 45070295 | 45070430 | Osteoblasts | 0.2 | 0.89 | intron | PRRS | U |
| 4852 | chr16 | 870620461 | 87062274 | Osteoblasts | 0.19 | 0.88 | Intergenic | C16orf95 | U |
| 4853 | chr7 | 1396534541 | 139653729 | Osteoblasts | 0.25 | 0.94 | intron | TBXAS1 | U |
| 4854 | chr11 | 46326464 | 46326700 | Osteoblasts | 0.21 | 0.89 | intron | CREB3L1 | U |
| 4855 | chr2 | 27173679 | 27174079 | Osteoblasts | 0.24 | 0.92 | TTS | DPYSL5 | U |
| 4856 | chr4 | 42123330 | 42123411 | Osteoblasts | 0.28 | 0.95 | intron | BEND4 | U |
| 4857 | chr19 | 3806500 | 3806614 | Osteoblasts | 0.22 | 0.89 | intron | ZFR2 | U |
| 4858 | chr7 | 1856511 | 1856721 | Osteoblasts | 0.19 | 0.86 | intron | MAD1L1 | U |
| 4859 | chr7 | 6633827 | 6634176 | Osteoblasts | 0.23 | 0.9 | intron | C7orf26 | U |
| 4860 | chr8 | 117595358 | 117595546 | Osteoblasts | 0.22 | 0.88 | Intergenic | EIF3H | U |
| 4861 | chr18 | 14090045 | 14090326 | Osteoblasts | 0.24 | 0.9 | intron | ZNF519 | U |
| 4862 | chr10 | 102787307 | 102787537 | Osteoblasts | 0.22 | 0.87 | intron | PDZD7 | U |
| 4863 | chr16 | 78433833 | 78434070 | Osteoblasts | 0.27 | 0.92 | intron | WWOX | U |
| 4864 | chr17 | 29804489 | 29804756 | Osteoblasts | 0.27 | 0.92 | intron | RAB11FIP4 | U |
| 4865 | chr11 | 13702816 | 13703244 | Osteoblasts | 0.29 | 0.94 | intron | FAR1 | U |
| 4866 | chr11 | 68425532 | 68425979 | Osteoblasts | 0.27 | 0.92 | Intergenic | GAL | U |
| 4867 | chr11 | 114085364 | 114085523 | Osteoblasts | 0.3 | 0.92 | intron | ZBTB16 | U |
| 4868 | chr3 | 1527640513 | 152764235 | Osteoblasts | 0.31 | 0.92 | Intergenic | RAP2B | U |
| 4869 | chr15 | 28423980 | 28424356 | Osteoblasts | 0.38 | 0.95 | intron | HERC2 | U |
| 4870 | chr6 | 71012146 | 71012302 | Osteoblasts | 0.03 | 0.91 | intron | COL9A1 | U |
| 4871 | chr10 | 130929379 | 130929518 | Osteoblasts | 0.08 | 0.92 | Intergenic | MGMT | U |
| 4872 | chr19 | 57850780 | 57850937 | Osteoblasts | 0.12 | 0.86 | Intergenic | ZNF304 | U |
| 4873 | chr6 | 71010703 | 71011017 | Osteoblasts | 0.05 | 0.88 | intron | COL9A1 | U |
| 4874 | chr21 | 33300962 | 33301128 | Osteoblasts | 0.07 | 0.88 | intron | HUNK | U |
| 4875 | chr16 | 87665731 | 87666123 | Osteoblasts | 0.04 | 0.83 | intron | JPH3 | U |
| 4876 | chr9 | 121658020 | 121658313 | Osteoblasts | 0.14 | 0.9 | Intergenic | DBC1 | U |
| 4877 | chr12 | 117630839 | 117630916 | Osteoblasts | 0.23 | 0.91 | Intergenic | FBXO21 | U |
| 4878 | chr22 | 25310719 | 25311022 | Osteoblasts | 0.02 | 0.94 | intron | SGSM1 | U |
| 4879 | chr17 | 25927592 | 25927656 | Osteoblasts | 0.08 | 0.96 | intron | KSR1 | U |
| 4880 | chr20 | 47056018 | 47056109 | Osteoblasts | 0.05 | 0.9 | Intergenic | LINC00494 | U |
| 4881 | chr6 | 3075149 | 3075314 | Osteoblasts | 0.15 | 0.89 | Intergenic | RIPK1 | U |
| 4882 | chr2 | 25456972 | 25457242 | Osteoblasts | 0.21 | 0.93 | exon | DNMT3A | U |
| 4883 | chr9 | 993163773 | 99316425 | Osteoblasts | 0.05 | 0.94 | intron | CDC14B | U |
| 4884 | chr9 | 94565110 | 94565267 | Osteoblasts | 0.09 | 0.91 | intron | ROR2 | U |
| 4885 | chr22 | 33403343 | 33403543 | Osteoblasts | 0.07 | 0.86 | promoter-TSS | SYN3 | U |
| 4886 | chr14 | 34085388 | 34085645 | Osteoblasts | 0.13 | 0.89 | intron | NPAS3 | U |
| 4887 | chr6 | 2414868 | 2415181 | Osteoblasts | 0.15 | 0.91 | TTS | LOC100508120 | U |
| 4888 | chr22 | 50054483 | 50054582 | Osteoblasts | 0.18 | 0.92 | Intergenic | C22orf34 | U |
| 4889 | chr20 | 11252715 | 11252998 | Osteoblasts | 0.14 | 0.88 | intron | LOC339593 | U |
| 4890 | chr9 | 98545897 | 98546074 | Osteoblasts | 0.18 | 0.9 | Intergenic | ERCC6L2 | U |
| 4891 | chr20 | 17905267 | 17905456 | Osteoblasts | 0.33 | 0.93 | Intergenic | SNORD17 | U |
| 4892 | chr18 | 77397505 | 77397593 | Osteoblasts | 0.86 | 0.16 | Intergenic | CTDP1 | M |
| 4893 | chr4 | 13533239 | 13533336 | Osteoblasts | 0.9 | 0.21 | promoter-TSS | LOC285547 | M |
| 4894 | chr4 | 13536606 | 13536659 | Osteoblasts | 0.8 | 0.16 | Intergenic | LOC285547 | M |
| 4895 | chr4 | 13535466 | 13535713 | Osteoblasts | 0.72 | 0.12 | Intergenic | LOC285547 | M |
| 4896 | chr4 | 13532927 | 13533188 | Osteoblasts | 0.55 | 0.07 | promoter-TSS | LOC285547 | M |
| 4897 | chr15 | 20779839 | 20780059 | Osteoblasts | 0.74 | 0.78 | exon | GOLGA8CP | M |
| 4898 | chr1 | 119542629 | 119543008 | Osteoblasts | 0.95 | 0.14 | Intergenic | TBX15 | M |
| 4899 | chr16 | 86612280 | 86612426 | Osteoblasts | 0.91 | 0.12 | exon | FOXL1 | M |
| 4900 | chr1 | 1195430881 | 119543454 | Osteoblasts | 0.87 | 0.08 | Intergenic | TBX15 | M |
| 4901 | chr18 | 77397614 | 77397745 | Osteoblasts | 0.94 | 0.16 | Intergenic | CTDP1 | M |
| 4902 | chr16 | 866131323 | 86613395 | Osteoblasts | 0.87 | 0.1 | exon | FOXL1 | M |
| 4903 | chr16 | 86610170 | 86610571 | Osteoblasts | 0.83 | 0.08 | Intergenic | FOXL1 | M |
| 4904 | chr7 | 27228664 | 27228806 | Osteoblasts | 0.84 | 0.12 | exon | HOXA11-AS | M |
| 4905 | chr19 | 18899000 | 18899055 | Osteoblasts | 0.86 | 0.18 | intron, exon | COMP, COMP | M |
| 4906 | chr12 | 114838313 | 114838411 | Osteoblasts | 0.82 | 0.14 | intron | TBX5 | M |
| 4907 | chr5 | 727401791 | 72740383 | Osteoblasts | 0.82 | 0.14 | Intergenic | FOXD1 | M |
| 4908 | chr12 | 114833665 | 114833985 | Osteoblasts | 0.74 | 0.09 | intron | TBX5 | M |
| 4909 | chr12 | 114838431 | 114838466 | Osteoblasts | 0.84 | 0.21 | intron | TBX5 | M |
| 4910 | chr4 | 13540614 | 13541000 | Osteoblasts | 0.71 | 0.09 | Intergenic | NKX3-2 | M |
| 4911 | chr4 | 13530923 | 13531028 | Osteoblasts | 0.75 | 0.15 | intron | LOC285547 | M |
| 4912 | chr4 | 13536483 | 13536583 | Osteoblasts | 0.62 | 0.08 | Intergenic | LOC285547 | M |
| 4913 | chr6 | 41341201 | 41341338 | Osteoblasts | 0.61 | 0.07 | Intergenic | NCR2 | M |
| 4914 | chr4 | 13535985 | 13536425 | Osteoblasts | 0.53 | 0.04 | Intergenic | LOC285547 | M |
| 4915 | chr19 | 18899119 | 18899510 | Osteoblasts | 0.53 | 0.07 | exon, intron | COMP, COMP | M |
| 4916 | chr2 | 176968736 | 176969226 | Osteoblasts | 0.51 | 0.07 | Intergenic | HOXD11 | M |
| 4917 | chr19 | 8203063 | 8203187 | Skeletal Muscle cells | 0.31 | 0.93 | exon | FBN3 | U |
| 4918 | chr1 | 556670043 | 55667230 | Skeletal Muscle cells | 0.35 | 0.94 | intron | USP24 | U |
| 4919 | chr19 | 3442501 | 3442685 | Skeletal Muscle cells | 0.34 | 0.92 | intron | NFIC | U |
| 4920 | chr20 | 48908487 | 48908567 | Skeletal Muscle cells | 0.32 | 0.89 | promoter-TSS | LOC284751 | U |
| 4921 | chr3 | 48127949 | 48128104 | Skeletal Muscle cells | 0.32 | 0.89 | intron | MAP4 | U |
| 4922 | chr10 | 23353063 | 23353386 | Skeletal Muscle cells | 0.36 | 0.92 | Intergenic | MSRB2 | U |
| 4923 | chr2 | 127072611 | 127073080 | Skeletal Muscle cells | 0.34 | 0.9 | Intergenic | GYPC | U |
| 4924 | chr2 | 1108061813 | 110806243 | Skeletal Muscle cells | 0.32 | 0.87 | Intergenic | MIR4267 | U |
| 4925 | chr15 | 98977214 | 98977282 | Skeletal Muscle cells | 0.33 | 0.88 | Intergenic | FAM1698 | U |
| 4926 | chr8 | 33401219 | 33401412 | Skeletal Muscle cells | 0.36 | 0.9 | Intergenic | RNF122 | U |
| 4927 | chr19 | 4893283 | 4893584 | Skeletal Muscle cells | 0.31 | 0.85 | intron | ARRDC5 | U |
| 4928 | chr2 | 1108064153 | 110806719 | Skeletal Muscle cells | 0.36 | 0.89 | Intergenic | MIR4267 | U |
| 4929 | chr4 | 1689116 | 1689174 | Skeletal Muscle cells | 0.35 | 0.87 | Intergenic | FAM53A | U |
| 4930 | chr1 | 1567243451 | 156724473 | Skeletal Muscle cells | 0.36 | 0.88 | Intergenic | HDGF | U |
| 4931 | chr17 | 5430619 | 5430879 | Skeletal Muscle cells | 0.39 | 0.91 | intron | NLRP1 | U |
| 4932 | chr16 | 21517221 | 21517585 | Skeletal Muscle cells | 0.37 | 0.89 | Intergenic | LOC100271836 | U |
| 4933 | chr19 | 490210903 | 49021217 | Skeletal Muscle cells | 0.35 | 0.86 | Intergenic | LMTK3 | U |
| 4934 | chr15 | 43566080 | 43566250 | Skeletal Muscle cells | 0.38 | 0.89 | Intergenic | TGM5 | U |
| 4935 | chr6 | 117596420 | 117596651 | Skeletal Muscle cells | 0.36 | 0.87 | Intergenic | VGLL2 | U |
| 4936 | chr20 | 55876271 | 55876308 | Skeletal Muscle cells | 0.3 | 0.79 | Intergenic | MIR4325 | U |
| 4937 | chr9 | 1400315211 | 140031628 | Skeletal Muscle cells | 0.39 | 0.84 | Intergenic | GRIN1 | U |
| 4938 | chr11 | 107584376 | 107584419 | Skeletal Muscle cells | 0.2 | 0.85 | Intergenic | SLN | U |
| 4939 | chr12 | 1311734671 | 131173598 | Skeletal Muscle cells | 0.31 | 0.93 | Intergenic | STX2 | U |
| 4940 | chr17 | 27967189 | 27967337 | Skeletal Muscle cells | 0.23 | 0.85 | intron | SSH2 | U |
| 4941 | chr19 | 45619325 | 45619559 | Skeletal Muscle cells | 0.26 | 0.88 | intron | PPP1R37 | U |
| 4942 | chr15 | 64186563 | 64186671 | Skeletal Muscle cells | 0.27 | 0.86 | Intergenic | HERC1 | U |
| 4943 | chr4 | 187826071 | 187826229 | Skeletal Muscle cells | 0.32 | 0.91 | Intergenic | FAT1 | U |
| 4944 | chr18 | 57863480 | 57863833 | Skeletal Muscle cells | 0.3 | 0.89 | Intergenic | MC4R | U |
| 4945 | chr15 | 619162493 | 61916272 | Skeletal Muscle cells | 0.3 | 0.88 | Intergenic | VPS13C | U |
| 4946 | chr15 | 42652008 | 42652187 | Skeletal Muscle cells | 0.31 | 0.89 | exon | CAPN3 | U |
| 4947 | chr2 | 2325535133 | 232553561 | Skeletal Muscle cells | 0.36 | 0.93 | Intergenic | PTMA | U |
| 4948 | chr6 | 336975593 | 33697652 | Skeletal Muscle cells | 0.34 | 0.91 | intron | IP6K3 | U |
| 4949 | chr13 | 1067442041 | 106744325 | Skeletal Muscle cells | 0.33 | 0.9 | Intergenic | LINC00460 | U |
| 4950 | chr2 | 234984056 | 234984232 | Skeletal Muscle cells | 0.31 | 0.88 | intron | SPP2 | U |
| 4951 | chr16 | 78936064 | 78936337 | Skeletal Muscle cells | 0.34 | 0.91 | intron | WWOX | U |
| 4952 | chr3 | 187724991 | 187725055 | Skeletal Muscle cells | 0.33 | 0.89 | Intergenic | LPP-AS2 | U |
| 4953 | chr15 | 41215757 | 41215904 | Skeletal Muscle cells | 0.34 | 0.9 | Intergenic | DLL4 | U |
| 4954 | chr2 | 177833296 | 177833478 | Skeletal Muscle cells | 0.27 | 0.83 | Intergenic | HNRNPA3 | U |
| 4955 | chr10 | 69881845 | 69882049 | Skeletal Muscle cells | 0.31 | 0.87 | exon | MYPN | U |
| 4956 | chr12 | 47086665 | 47086941 | Skeletal Muscle cells | 0.34 | 0.9 | Intergenic | SLC38A4 | U |
| 4957 | chr3 | 15540003 | 15540161 | Skeletal Muscle cells | 0.3 | 0.85 | exon | COLQ | U |
| 4958 | chr11 | 1993784 | 1993963 | Skeletal Muscle cells | 0.36 | 0.91 | Intergenic | MRPL23-AS1 | U |
| 4959 | chr18 | 633846 | 633900 | Skeletal Muscle cells | 0.38 | 0.92 | intron | CLUL1 | U |
| 4960 | chr12 | 122848458 | 122848547 | Skeletal Muscle cells | 0.36 | 0.9 | intron | CLIP1 | U |
| 4961 | chr21 | 44857515 | 44857623 | Skeletal Muscle cells | 0.36 | 0.9 | Intergenic | SIK1 | U |
| 4962 | chr17 | 4528360 | 4528541 | Skeletal Muscle cells | 0.36 | 0.9 | Intergenic | ALOX15 | U |
| 4963 | chr8 | 144551748 | 144551942 | Skeletal Muscle cells | 0.35 | 0.89 | intron | ZC3H3 | U |
| 4964 | chr2 | 218735522 | 218735734 | Skeletal Muscle cells | 0.35 | 0.89 | intron | TNS1 | U |
| 4965 | chr19 | 50347798 | 50348017 | Skeletal Muscle cells | 0.31 | 0.85 | exon | PTOV1-AS1 | U |
| 4966 | chr21 | 19642209 | 19642491 | Skeletal Muscle cells | 0.33 | 0.87 | exon, intron | TMPRSS15, TMPRSS1 [symbol] | U |
| 4967 | chr7 | 36250051 | 36250343 | Skeletal Muscle cells | 0.3 | 0.84 | intron | EEPD1 | U |
| 4968 | chr3 | 84954050 | 84954379 | Skeletal Muscle cells | 0.3 | 0.84 | Intergenic | LOC440970 | U |
| 4969 | chr10 | 62608802 | 62609231 | Skeletal Muscle cells | 0.35 | 0.89 | Intergenic | CDK1 | U |
| 4970 | chr17 | 65040029 | 65040463 | Skeletal Muscle cells | 0.34 | 0.88 | promoter-TSS | CACNG1 | U |
| 4971 | chr4 | 148927308 | 148927474 | Skeletal Muscle cells | 0.34 | 0.87 | intron | ARHGAP10 | U |
| 4972 | chr4 | 6230304 | 6230371 | Skeletal Muscle cells | 0.3 | 0.82 | intron | LOC285484 | U |
| 4973 | chr7 | 187369 | 187453 | Skeletal Muscle cells | 0.36 | 0.88 | Intergenic | FAM20C | U |
| 4974 | chr2 | 179632508 | 1796326201 | Skeletal Muscle cells | 0.34 | 0.86 | exon | TTN | U |
| 4975 | chr7 | 152397270 | 152397439 | Skeletal Muscle cells | 0.34 | 0.86 | Intergenic | XRCC2 | U |
| 4976 | chr3 | 42726949 | 42727134 | Skeletal Muscle cells | 0.3 | 0.82 | promoter-TSS | KLHL40 | U |
| 4977 | chr2 | 1690267 | 1690466 | Skeletal Muscle cells | 0.32 | 0.84 | intron | PXDN | U |
| 4978 | chr7 | 127858051 | 127858403 | Skeletal Muscle cells | 0.28 | 0.8 | Intergenic | MIR129-1 | U |
| 4979 | chr12 | 57644425 | 57644780 | Skeletal Muscle cells | 0.27 | 0.79 | intron | STAC3 | U |
| 4980 | chr2 | 85659668 | 85660162 | Skeletal Muscle cells | 0.37 | 0.89 | Intergenic | SH2D6 | U |
| 4981 | chr1 | 196619616 | 196619773 | Skeletal Muscle cells | 0.34 | 0.85 | Intergenic | CFH | U |
| 4982 | chr11 | 119219095 | 119219309 | Skeletal Muscle cells | 0.32 | 0.83 | Intergenic | C1QTNF5 | U |
| 4983 | chr1 | 199357175 | 199357536 | Skeletal Muscle cells | 0.36 | 0.87 | Intergenic | LOC100131234 | U |
| 4984 | chr16 | 87004646 | 87004878 | Skeletal Muscle cells | 0.37 | 0.87 | Intergenic | C16orf95 | U |
| 4985 | chr1 | 115727622 | 115727898 | Skeletal Muscle cells | 0.35 | 0.85 | Intergenic | TSPAN2 | U |
| 4986 | chr15 | 61916027 | 61916136 | Skeletal Muscle cells | 0.31 | 0.8 | Intergenic | VPS13C | U |
| 4987 | chr22 | 48977209 | 48977383 | Skeletal Muscle cells | 0.35 | 0.84 | intron | FAM19A5 | U |
| 4988 | chr11 | 61477123 | 61477312 | Skeletal Muscle cells | 0.31 | 0.8 | intron | DAGLA | U |
| 4989 | chr4 | 137276273 | 137276479 | Skeletal Muscle cells | 0.37 | 0.86 | Intergenic | PCDH18 | U |
| 4990 | chr5 | 31869910 | 31870233 | Skeletal Muscle cells | 0.36 | 0.85 | intron | PDZD2 | U |
| 4991 | chr10 | 128959870 | 128960264 | Skeletal Muscle cells | 0.35 | 0.84 | intron | FAM196A | U |
| 4992 | chr1 | 118832980 | 118833398 | Skeletal Muscle cells | 0.34 | 0.83 | Intergenic | SPAG17 | U |
| 4993 | chr17 | 78460515 | 78460559 | Skeletal Muscle cells | 0.27 | 0.75 | Intergenic | NPTX1 | U |
| 4994 | chr19 | 32808404 | 32808566 | Skeletal Muscle cells | 0.34 | 0.82 | Intergenic | ZNF507 | U |
| 4995 | chr1 | 53953114 | 53953284 | Skeletal Muscle cells | 0.37 | 0.85 | Intergenic | DMRT81 | U |
| 4996 | chr2 | 242152815 | 242153018 | Skeletal Muscle cells | 0.36 | 0.84 | intron | ANO7 | U |
| 4997 | chr2 | 234242884 | 234243117 | Skeletal Muscle cells | 0.36 | 0.84 | intron | SAG | U |
| 4998 | chr4 | 187874572 | 187874999 | Skeletal Muscle cells | 0.39 | 0.87 | Intergenic | FAT1 | U |
| 4999 | chr15 | 27702726 | 27702873 | Skeletal Muscle cells | 0.37 | 0.84 | intron | GABRG3 | U |
| 5000 | chr15 | 65698874 | 65699074 | Skeletal Muscle cells | 0.35 | 0.82 | intron | IGDCC4 | U |
| 5001 | chr15 | 87030198 | 87030480 | Skeletal Muscle cells | 0.38 | 0.85 | intron | AGBL1 | U |
| 5002 | chr17 | 13365740 | 13366160 | Skeletal Muscle cells | 0.35 | 0.82 | Intergenic | HS3ST3A1 | U |
| 5003 | chr17 | 17550190 | 17550422 | Skeletal Muscle cells | 0.33 | 0.79 | Intergenic | RAI1 | U |
| 5004 | chr13 | 97912473 | 97912720 | Skeletal Muscle cells | 0.38 | 0.84 | intron | MBNL2 | U |
| 5005 | chr18 | 47370091 | 47370400 | Skeletal Muscle cells | 0.37 | 0.83 | intron | MYO5B | U |
| 5006 | chr8 | 58130870 | 58131180 | Skeletal Muscle cells | 0.37 | 0.83 | promoter-TSS | LOC100507651 | U |
| 5007 | chr10 | 53061543 | 53061963 | Skeletal Muscle cells | 0.38 | 0.84 | intron | PRKG1 | U |
| 5008 | chr6 | 169779678 | 169779870 | Skeletal Muscle cells | 0.37 | 0.82 | Intergenic | THBS2 | U |
| 5009 | chr1 | 910433 | 910476 | Skeletal Muscle cells | 0.37 | 0.81 | TTS | PLEKHN1 | U |
| 5010 | chr3 | 195489940 | 195490218 | Skeletal Muscle cells | 0.37 | 0.8 | intron | MUC4 | U |
| 5011 | chr2 | 118892245 | 118892609 | Skeletal Muscle cells | 0.39 | 0.82 | Intergenic | INSIG2 | U |
| 5012 | chr2 | 219696690 | 219697160 | Skeletal Muscle cells | 0.39 | 0.82 | promoter-TSS | PRKAG3 | U |
| 5013 | chr18 | 76517219 | 76517285 | Skeletal Muscle cells | 0.36 | 0.78 | Intergenic | SALL3 | U |
| 5014 | chr12 | 3118961 | 3119134 | Skeletal Muscle cells | 0.37 | 0.79 | intron | TEAD4 | U |
| 5015 | chr19 | 4561919 | 4562055 | Skeletal Muscle cells | 0.37 | 0.78 | Intergenic | SEMA6B | U |
| 5016 | chr2 | 3050453 | 3050602 | Skeletal Muscle cells | 0.38 | 0.78 | Intergenic | FSSC1 | U |
| 5017 | chr12 | 117878550 | 117878698 | Skeletal Muscle cells | 0.36 | 0.87 | Intergenic | NOS1 | U |
| 5018 | chr6 | 152664348 | 152664590 | Skeletal Muscle cells | 0.34 | 0.9 | intron | SYNE1 | U |
| 5019 | chr5 | 38046369 | 38046580 | Skeletal Muscle cells | 0.36 | 0.88 | Intergenic | GDNF | U |
| 5020 | chr1 | 229572271 | 229572423 | Skeletal Muscle cells | 0.36 | 0.85 | Intergenic | ACTA1 | U |
| 5021 | chr4 | 134357530 | 134357673 | Skeletal Muscle cells | 0.36 | 0.84 | Intergenic | PCDH10 | U |
| 5022 | chr1 | 240689970 | 240690130 | Skeletal Muscle cells | 0.37 | 0.84 | intron | GREM2 | U |
| 5023 | chr1 | 229544900 | 229545255 | Skeletal Muscle cells | 0.39 | 0.85 | Intergenic | ACTA1 | U |
| 5024 | chr14 | 20904169 | 20904320 | Skeletal Muscle cells | 0.32 | 0.92 | promoter-TSS | KLHL33 | U |
| 5025 | chr9 | 84081386 | 84081501 | Skeletal Muscle cells | 0.31 | 0.89 | Intergenic | TLE1 | U |
| 5026 | chr8 | 58130236 | 58130521 | Skeletal Muscle cells | 0.38 | 0.9 | Intergenic | LOC100507651 | U |
| 5027 | chr14 | 20903959 | 20904321 | Skeletal Muscle cells | 0.32 | 0.9 | promoter-TSS | KLHL33 | U |
| 5028 | chr14 | 584101441 | 58410459 | Skeletal Muscle cells | 0.28 | 0.84 | Intergenic | SLC35F4 | U |
| 5029 | chr6 | 36336696 | 36336879 | Skeletal Muscle cells | 0.35 | 0.89 | exon | ETV7 | U |
| 5030 | chr9 | 74426982 | 74427329 | Skeletal Muscle cells | 0.35 | 0.89 | Intergenic | TMEM2 | U |
| 5031 | chr20 | 20065420 | 20065874 | Skeletal Muscle cells | 0.33 | 0.87 | intron | C20orf26 | U |
| 5032 | chr6 | 17296804 | 17297082 | Skeletal Muscle cells | 0.35 | 0.88 | Intergenic | RBM24 | U |
| 5033 | chr19 | 5507442 | 5507541 | Skeletal Muscle cells | 0.29 | 0.81 | Intergenic | ZNRF4 | U |
| 5034 | chr5 | 3489600 | 3489849 | Skeletal Muscle cells | 0.29 | 0.81 | intron | LOC285577 | U |
| 5035 | chr18 | 76151431 | 76151522 | Skeletal Muscle cells | 0.37 | 0.88 | Intergenic | SALL3 | U |
| 5036 | chr11 | 2048934 | 2049033 | Skeletal Muscle cells | 0.39 | 0.87 | Intergenic | H19 | U |
| 5037 | chr8 | 58130560 | 58130672 | Skeletal Muscle cells | 0.36 | 0.81 | Intergenic | LOC100507651 | U |
| 5038 | chr22 | 18915145 | 18915393 | Skeletal Muscle cells | 0.38 | 0.83 | intron | PRODH | U |
| 5039 | chr22 | 39580653 | 39580954 | Skeletal Muscle cells | 0.38 | 0.82 | Intergenic | CBX7 | U |
| 5040 | chr6 | 5843289 | 5843661 | Skeletal Muscle cells | 0.35 | 0.79 | Intergenic | NRN1 | U |
| 5041 | chr9 | 133308491 | 133308595 | Skeletal Muscle cells | 0.71 | 0.17 | Intergenic | ASS1 | M |
| 5042 | chr19 | 39055700 | 39055814 | Skeletal Muscle cells | 0.57 | 0.07 | exon | RYR1 | M |
| 5043 | chr4 | 89618840 | 89618862 | Skeletal Muscle cells | 0.79 | 0.45 | exon | NAP1L5 | M |
| 5044 | chr2 | 223153657 | 223153970 | Skeletal Muscle cells | 0.67 | 0.15 | intron | PAX3 | M |
| 5045 | chr6 | 100902882 | 100903073 | Skeletal Muscle cells | 0.66 | 0.19 | intron | SIM1 | M |
| 5046 | chr2 | 239072766 | 239072927 | Skeletal Muscle cells | 0.72 | 0.09 | Intergenic | KLHL30 | M |
| 5047 | chr1 | 119522486 | 119522674 | Skeletal Muscle cells | 0.72 | 0.11 | intron | TBX15 | M |
| 5048 | chr10 | 102975459 | 102975755 | Skeletal Muscle cells | 0.63 | 0.07 | Intergenic | LBX1 | M |
| 5049 | chr13 | 79181211 | 79181333 | Skeletal Muscle cells | 0.64 | 0.13 | exon | POU4F1-ASI | M |
| 5050 | chr19 | 2251365 | 2251656 | Skeletal Muscle cells | 0.63 | 0.12 | TTS | JSRP1 | M |
| 5051 | chr15 | 68127629 | 68127976 | Skeletal Muscle cells | 0.58 | 0.08 | Intergenic | SKOR1 | M |
| 5052 | chr10 | 102983359 | 102983478 | Skeletal Muscle cells | 0.58 | 0.09 | Intergenic | LBX1 | M |
| 5053 | chr2 | 223165479 | 223165799 | Skeletal Muscle cells | 0.57 | 0.09 | intron | CCDC140 | M |
| 5054 | chr10 | 1029782523 | 102978631 | Skeletal Muscle cells | 0.6 | 0.12 | Intergenic | LBX1 | M |
| 5055 | chr2 | 2231662863 | 223166343 | Skeletal Muscle cells | 0.65 | 0.18 | intron | CCDC140 | M |
| 5056 | chr6 | 100904161 | 100904317 | Skeletal Muscle cells | 0.6 | 0.13 | intron | SIM1 | M |
| 5057 | chr10 | 102982888 | 102983161 | Skeletal Muscle cells | 0.54 | 0.08 | Intergenic | LBX1 | M |
| 5058 | chr3 | 147106635 | 147107083 | Skeletal Muscle cells | 0.58 | 0.12 | intron | ZIC4 | M |
| 5059 | chr6 | 100903707 | 100904127 | Skeletal Muscle cells | 0.53 | 0.09 | intron | SIM1 | M |
| 5060 | chr10 | 102975233 | 102975392 | Skeletal Muscle cells | 0.55 | 0.12 | Intergenic | LBX1 | M |
| 5061 | chr10 | 102996412 | 102996511 | Skeletal Muscle cells | 0.57 | 0.15 | intron | FLI41350 | M |
| 5062 | chr2 | 2231693383 | 223169478 | Skeletal Muscle cells | 0.53 | 0.13 | exon | CCDC140 | M |
| 5063 | chr11 | 20229033 | 20229218 | Skeletal Muscle cells | 0.54 | 0.14 | Intergenic | DBX1 | M |
| 5064 | chr6 | 134214113 | 134214135 | Skeletal Muscle cells | 0.57 | 0.18 | TTS | TCF21 | M |
| 5065 | chr1 | 162472870 | 162473127 | Smooth Muscle cells | 0.15 | 0.92 | intron | UHMK1 | U |
| 5066 | chr1 | 19668409 | 19668671 | Smooth Muscle cells | 0.14 | 0.85 | intron | CAPZB | U |
| 5067 | chr13 | 85761481 | 85761897 | Smooth Muscle cells | 0.2 | 0.87 | Intergenic | LINC00351 | U |
| 5068 | chr12 | 93346409 | 93346508 | Smooth Muscle cells | 0.22 | 0.87 | Intergenic | EEA1 | U |
| 5069 | chr5 | 105502349 | 105502820 | Smooth Muscle cells | 0.14 | 0.78 | Intergenic | RAB9BP1 | U |
| 5070 | chr5 | 150054296 | 150054520 | Smooth Muscle cells | 0.26 | 0.89 | intron | MYOZ3 | U |
| 5071 | chr17 | 27924735 | 27925011 | Smooth Muscle cells | 0.26 | 0.86 | intron | ANKRD13B | U |
| 5072 | chr4 | 187344569 | 187344895 | Smooth Muscle cells | 0.29 | 0.88 | intron | LOC285441 | U |
| 5073 | chr1 | 4329526 | 4329591 | Smooth Muscle cells | 0.26 | 0.81 | Intergenic | LOC284661 | U |
| 5074 | chr11 | 41437638 | 41438037 | Smooth Muscle cells | 0.25 | 0.8 | intron | LRRC4C | U |
| 5075 | chrX | 37870948 | 37871245 | Smooth Muscle cells | 0.25 | 0.79 | intron | SYTL5 | U |
| 5076 | chr2 | 239618443 | 239618634 | Smooth Muscle cells | 0.27 | 0.8 | Intergenic | TWIST2 | U |
| 5077 | chr9 | 1084974 | 1085359 | Smooth Muscle cells | 0.34 | 0.86 | Intergenic | DMRT2 | U |
| 5078 | chr18 | 18785355 | 18785725 | Smooth Muscle cells | 0.31 | 0.81 | Intergenic | GREB1L | U |
| 5079 | chr9 | 132085461 | 132085596 | Smooth Muscle cells | 0.32 | 0.82 | TTS | C9orf106 | U |
| 5080 | chr19 | 33279635 | 33280113 | Smooth Muscle cells | 0.33 | 0.83 | intron | TDRD12 | U |
| 5081 | chr8 | 1271433173 | 127143534 | Smooth Muscle cells | 0.34 | 0.83 | Intergenic | LOC100130231 | U |
| 5082 | chr7 | 159083175 | 159083533 | Smooth Muscle cells | 0.3 | 0.78 | Intergenic | VIPR2 | U |
| 5083 | chr16 | 2249613 | 2249795 | Smooth Muscle cells | 0.32 | 0.77 | Intergenic | CASKIN1 | U |
| 5084 | chr2 | 2416843741 | 241684584 | Smooth Muscle cells | 0.33 | 0.78 | intron | KIF1A | U |
| 5085 | chr2 | 234106430 | 234106749 | Smooth Muscle cells | 0.35 | 0.79 | intron | INPP5D | U |
| 5086 | chr1 | 42621305 | 42621502 | Smooth Muscle cells | 0.33 | 0.77 | TTS | GUCA2B | U |
| 5087 | chr15 | 61264553 | 61264728 | Smooth Muscle cells | 0.12 | 0.84 | intron | RORA | U |
| 5088 | chr1 | 19668811 | 19669130 | Smooth Muscle cells | 0.16 | 0.87 | Intron | CAPZB | U |
| 5089 | chr16 | 60313084 | 60313179 | Smooth Muscle cells | 0.19 | 0.85 | Intergenic | LOC644649 | U |
| 5090 | chr20 | 59234431 | 59234489 | Smooth Muscle cells | 0.19 | 0.84 | Intergenic | MIR4533 | U |
| 5091 | chr16 | 79167187 | 79167363 | Smooth Muscle cells | 0.24 | 0.88 | intron | WWOX | U |
| 5092 | chr11 | 117755919 | 117756301 | Smooth Muscle cells | 0.24 | 0.88 | Intergenic | FXYD6-FXYD2 | U |
| 5093 | chr15 | 53729197 | 53729635 | Smooth Muscle cells | 0.19 | 0.83 | Intergenic | WDR72 | U |
| 5094 | chr11 | 38195199 | 38195382 | Smooth Muscle cells | 0.21 | 0.84 | Intergenic | RAG2 | U |
| 5095 | chr1 | 3999997 | 4000145 | Smooth Muscle cells | 0.19 | 0.81 | promoter-TSS | LOC728716 | U |
| 5096 | chr18 | 70791867 | 70792080 | Smooth Muscle cells | 0.21 | 0.83 | Intergenic | LOC400655 | U |
| 5097 | chr16 | 4313460 | 4313710 | Smooth Muscle cells | 0.22 | 0.84 | intron | TFAP4 | U |
| 5098 | chr21 | 35231278 | 35231433 | Smooth Muscle cells | 0.24 | 0.85 | intron | ITSN1 | U |
| 5099 | chr19 | 5926434 | 5926755 | Smooth Muscle cells | 0.24 | 0.85 | intron | RANBP3 | U |
| 5100 | chr9 | 135414930 | 135415076 | Smooth Muscle cells | 0.19 | 0.79 | intron | C9orf171 | U |
| 5101 | chr3 | 560079023 | 56008341 | Smooth Muscle cells | 0.25 | 0.86 | intron | ERC2 | U |
| 5102 | chr16 | 74303746 | 74303946 | Smooth Muscle cells | 0.28 | 0.87 | Intergenic | PSMD7 | U |
| 5103 | chr17 | 606964501 | 60696713 | Smooth Muscle cells | 0.28 | 0.87 | Intergenic | MRC2 | U |
| 5104 | chr8 | 286667 | 286882 | Smooth Muscle cells | 0.27 | 0.85 | Intergenic | FBXO25 | U |
| 5105 | chr17 | 10100002 | 10100239 | Smooth Muscle cells | 0.26 | 0.84 | intron | GAS7 | U |
| 5106 | chr6 | 43711267 | 43711738 | Smooth Muscle cells | 0.29 | 0.87 | Intergenic | VEGFA | U |
| 5107 | chr20 | 8658174 | 3658389 | Smooth Muscle cells | 0.21 | 0.78 | intron | ADAM33 | U |
| 5108 | chr16 | 79238285 | 79238502 | Smooth Muscle cells | 0.27 | 0.84 | intron | WWOX | U |
| 5109 | chr2 | 691790 | 692052 | Smooth Muscle cells | 0.3 | 0.87 | Intergenic | TMEM18 | U |
| 5110 | chr16 | 29210481 | 29210814 | Smooth Muscle cells | 0.3 | 0.87 | Intergenic | SNX29P2 | U |
| 5111 | chrX | 68254156 | 68254588 | Smooth Muscle cells | 0.29 | 0.86 | Intergenic | PJA1 | U |
| 5112 | chr10 | 134845366 | 134845556 | Smooth Muscle cells | 0.25 | 0.81 | Intergenic | GPR123 | U |
| 5113 | chr11 | 59613940 | 59614213 | Smooth Muscle cells | 0.27 | 0.83 | Intergenic | GIF | U |
| 5114 | chr19 | 31131014 | 31131195 | Smooth Muscle cells | 0.29 | 0.84 | Intergenic | ZNF536 | U |
| 5115 | chr2 | 16266027 | 16266345 | Smooth Muscle cells | 0.26 | 0.81 | Intergenic | MYCNOS | U |
| 5116 | chr14 | 97923916 | 97924046 | Smooth Muscle cells | 0.29 | 0.83 | Intergenic | LOC100129345 | U |
| 5117 | chr15 | 26962426 | 26962831 | Smooth Muscle cells | 0.28 | 0.82 | promoter-TSS | GABRB3 | U |
| 5118 | chr14 | 1040013153 | 104001782 | Smooth Muscle cells | 0.29 | 0.83 | exon | TRMT61A | U |
| 5119 | chr4 | 187424572 | 187425045 | Smooth Muscle cells | 0.29 | 0.83 | Intergenic | LOC285441 | U |
| 5120 | chr22 | 43946806 | 43947015 | Smooth Muscle cells | 0.3 | 0.83 | intron | EFCAB6 | U |
| 5121 | chr3 | 53722379 | 53722607 | Smooth Muscle cells | 0.28 | 0.81 | intron | CACNA1D | U |
| 5122 | chr2 | 1231898 | 1232145 | Smooth Muscle cells | 0.26 | 0.79 | intron | SNTG2 | U |
| 5123 | chr12 | 53458465 | 53458841 | Smooth Muscle cells | 0.32 | 0.85 | TTS | TENC1 | U |
| 5124 | chr11 | 131316805 | 131317191 | Smooth Muscle cells | 0.25 | 0.78 | intron | NTM | U |
| 5125 | chr7 | 7011930 | 7012353 | Smooth Muscle cells | 0.29 | 0.82 | Intergenic | LOC100131257 | U |
| 5126 | chr20 | 60441678 | 60441790 | Smooth Muscle cells | 0.28 | 0.8 | intron | CDH4 | U |
| 5127 | chr2 | 8116225 | 8116503 | Smooth Muscle cells | 0.28 | 0.8 | intron | LOC339788 | U |
| 5128 | chr5 | 298575 | 298952 | Smooth Muscle cells | 0.28 | 0.8 | intron | PDCD6 | U |
| 5129 | chr4 | 139578057 | 139578485 | Smooth Muscle cells | 0.32 | 0.84 | Intergenic | LINC00499 | U |
| 5130 | chr7 | 45299101 | 4530342 | Smooth Muscle cells | 0.26 | 0.78 | Intergenic | FOXK1 | U |
| 5131 | chr1 | 239071144 | 239071596 | Smooth Muscle cells | 0.3 | 0.82 | Intergenic | LOC339535 | U |
| 5132 | chr5 | 3297915 | 3298378 | Smooth Muscle cells | 0.29 | 0.81 | Intergenic | LOC285577 | U |
| 5133 | chr12 | 119858815 | 119858904 | Smooth Muscle cells | 0.26 | 0.77 | intron | CCDC60 | U |
| 5134 | chr18 | 36480723 | 36481033 | Smooth Muscle cells | 0.27 | 0.78 | Intergenic | MIR5583-1 | U |
| 5135 | chr16 | 5546289 | 5546607 | Smooth Muscle cells | 0.29 | 0.8 | Intergenic | FAM86A | U |
| 5136 | chr5 | 53122510 | 53122874 | Smooth Muscle cells | 0.33 | 0.84 | Intergenic | MIR581 | U |
| 5137 | chr2 | 157516784 | 157517172 | Smooth Muscle cells | 0.31 | 0.82 | Intergenic | GPD2 | U |
| 5138 | chr7 | 4581687 | 4582108 | Smooth Muscle cells | 0.32 | 0.83 | Intergenic | FOXK1 | U |
| 5139 | chr14 | 104503689 | 104503821 | Smooth Muscle cells | 0.3 | 0.8 | intron | TDRD9 | U |
| 5140 | chr2 | 126895975 | 126896193 | Smooth Muscle cells | 0.32 | 0.82 | Intergenic | GYPC | U |
| 5141 | chr6 | 170409103 | 170409328 | Smooth Muscle cells | 0.24 | 0.74 | Intergenic | LOC154449 | U |
| 5142 | chr15 | 62017772 | 62018000 | Smooth Muscle cells | 0.28 | 0.78 | Intergenic | VPS13C | U |
| 5143 | chr11 | 62167036 | 62167371 | Smooth Muscle cells | 0.31 | 0.81 | Intergenic | SCGB1A1 | U |
| 5144 | chr15 | 61545575 | 61545932 | Smooth Muscle cells | 0.36 | 0.86 | Intergenic | RORA | U |
| 5145 | chr11 | 59989577 | 59990004 | Smooth Muscle cells | 0.32 | 0.82 | Intergenic | MS4A6A | U |
| 5146 | chr7 | 21427949 | 21428417 | Smooth Muscle cells | 0.33 | 0.83 | Intergenic | SP4 | U |
| 5147 | chr1 | 96105828 | 96106182 | Smooth Muscle cells | 0.31 | 0.8 | Intergenic | FLI31662 | U |
| 5148 | chr7 | 157665797 | 157666012 | Smooth Muscle cells | 0.35 | 0.83 | intron | PTPRN2 | U |
| 5149 | chr16 | 6864852 | 6865069 | Smooth Muscle cells | 0.35 | 0.83 | intron | RBFOX1 | U |
| 5150 | chr7 | 154389079 | 154389364 | Smooth Muscle cells | 0.33 | 0.81 | intron | DPP6 | U |
| 5151 | chr2 | 16119310 | 16119640 | Smooth Muscle cells | 0.3 | 0.78 | Intergenic | MYCNOS | U |
| 5152 | chr17 | 31745526 | 31745960 | Smooth Muscle cells | 0.29 | 0.77 | intron | ASIC2 | U |
| 5153 | chr13 | 68453611 | 68454048 | Smooth Muscle cells | 0.33 | 0.81 | Intergenic | PCDH9 | U |
| 5154 | chr7 | 157667235 | 157667336 | Smooth Muscle cells | 0.34 | 0.81 | intron | PTPRN2 | U |
| 5155 | chr14 | 98724094 | 98724302 | Smooth Muscle cells | 0.36 | 0.83 | Intergenic | C14orf64 | U |
| 5156 | chr13 | 70710716 | 70711087 | Smooth Muscle cells | 0.33 | 0.8 | intron | ATXN8OS | U |
| 5157 | chr12 | 109477369 | 109477694 | Smooth Muscle cells | 0.33 | 0.78 | Intergenic | USP30 | U |
| 5158 | chr1 | 166754379 | 166754739 | Smooth Muscle cells | 0.35 | 0.8 | Intergenic | POGK | U |
| 5159 | chr14 | 97715471 | 97715839 | Smooth Muscle cells | 0.34 | 0.79 | Intergenic | LOC100129345 | U |
| 5160 | chr13 | 53517414 | 53517793 | Smooth Muscle cells | 0.35 | 0.8 | Intergenic | OLFM4 | U |
| 5161 | chr3 | 194756583 | 194756647 | Smooth Muscle cells | 0.31 | 0.75 | Intergenic | XXYLT1 | U |
| 5162 | chr8 | 10825656 | 10825979 | Smooth Muscle cells | 0.35 | 0.79 | intron | XKR6 | U |
| 5163 | chr3 | 117987970 | 117988358 | Smooth Muscle cells | 0.35 | 0.79 | Intergenic | IGSF11-AS1 | U |
| 5164 | chr2 | 153958 | 154414 | Smooth Muscle cells | 0.34 | 0.78 | Intergenic | FAM110C | U |
| 5165 | chr7 | 157823927 | 157824386 | Smooth Muscle cells | 0.34 | 0.78 | intron | PTPRN2 | U |
| 5166 | chr1 | 111040980 | 111041120 | Smooth Muscle cells | 0.34 | 0.77 | Intergenic | CYMP | U |
| 5167 | chr3 | 97353123 | 97353445 | Smooth Muscle cells | 0.33 | 0.76 | intron | EPHAG | U |
| 5168 | chr11 | 39890287 | 39890688 | Smooth Muscle cells | 0.33 | 0.76 | Intergenic | LRRC4C | U |
| 5169 | chr12 | 34029238 | 34029378 | Smooth Muscle cells | 0.38 | 0.8 | Intergenic | ALG10 | U |
| 5170 | chr1 | 35213151 | 35213428 | Smooth Muscle cells | 0.3 | 0.72 | Intergenic | GJB5 | U |
| 5171 | chr10 | 1903252 | 1903566 | Smooth Muscle cells | 0.35 | 0.77 | Intergenic | ADARB2 | U |
| 5172 | chr8 | 102324166 | 102324500 | Smooth Muscle cells | 0.36 | 0.77 | Intergenic | NACAP1 | U |
| 5173 | chr22 | 47651354 | 47651596 | Smooth Muscle cells | 0.34 | 0.74 | Intergenic | FLI46257 | U |
| 5174 | chr3 | 128440062 | 128440165 | Smooth Muscle cells | 0.34 | 0.73 | Intergenic | RAB7A | U |
| 5175 | chr1 | 37868647 | 37868988 | Smooth Muscle cells | 0.35 | 0.73 | Intergenic | LOC728431 | U |
| 5176 | chr10 | 38802268 | 38802537 | Smooth Muscle cells | 0.35 | 0.72 | Intergenic | LOC399744 | U |
| 5177 | chr2 | 2429578143 | 242958199 | Smooth Muscle cells | 0.36 | 0.73 | Intergenic | LOC728323 | U |
| 5178 | chr9 | 126755520 | 126755782 | Smooth Muscle cells | 0.37 | 0.72 | Intergenic | LHX2 | U |
| 5179 | chr16 | 68767970 | 68768224 | Smooth Muscle cells | 0.37 | 0.87 | Intergenic | CDH1 | U |
| 5180 | chr4 | 38426698 | 38427116 | Smooth Muscle cells | 0.31 | 0.84 | Intergenic | KLF3 | U |
| 5181 | chrX | 90908496 | 90908836 | Smooth Muscle cells | 0.31 | 0.78 | Intergenic | PCDH11X | U |
| 5182 | chr12 | 122206348 | 122206441 | Smooth Muscle cells | 0.17 | 0.87 | intron | TMEM1208 | U |
| 5183 | chr14 | 28514717 | 28514969 | Smooth Muscle cells | 0.36 | 0.81 | Intergenic | LINC00645 | U |
| 5184 | chr7 | 28887244 | 28887569 | Smooth Muscle cells | 0.15 | 0.82 | Intergenic | TRIL | U |
| 5185 | chr9 | 104083380 | 104083851 | Smooth Muscle cells | 0.24 | 0.85 | intron | LPPR1 | U |
| 5186 | chr19 | 10928691 | 10929142 | Smooth Muscle cells | 0.25 | 0.85 | promoter-TSS | MIR199A1 | U |
| 5187 | chr9 | 18012503 | 18012777 | Smooth Muscle cells | 0.26 | 0.85 | Intergenic | SH3GL2 | U |
| 5188 | chr22 | 29589074 | 29589379 | Smooth Muscle cells | 0.28 | 0.86 | Intergenic | EMID1 | U |
| 5189 | chr14 | 36921436 | 36921877 | Smooth Muscle cells | 0.3 | 0.82 | Intergenic | SFTA3 | U |
| 5190 | chr9 | 104450777 | 104451030 | Smooth Muscle cells | 0.3 | 0.8 | intron | GRIN3A | U |
| 5191 | chr14 | 70670452 | 70670892 | Smooth Muscle cells | 0.33 | 0.73 | Intergenic | SLC8A3 | U |
| 5192 | chr8 | 26048065 | 26048282 | Smooth Muscle cells | 0.81 | 0.16 | Intergenic | PPP2R2A | M |
| 5193 | chr8 | 26047813 | 26047895 | Smooth Muscle cells | 0.75 | 0.15 | Intergenic | PPP2R2A | M |
| 5194 | chr8 | 143483475 | 143483622 | Smooth Muscle cells | 0.62 | 0.11 | intron | TSNARE1 | M |
| 5195 | chr8 | 72757629 | 72757788 | Smooth Muscle cells | 0.7 | 0.19 | promoter-TSS | MSC | M |
| 5196 | chr7 | 73242001 | 73242259 | Smooth Muscle cells | 0.68 | 0.19 | Intergenic | CLDN4 | M |
| 5197 | chr22 | 50587574 | 50587895 | Smooth Muscle cells | 0.65 | 0.16 | intron | MOV10L1 | M |
| 5198 | chr2 | 191273996 | 191274078 | Smooth Muscle cells | 0.63 | 0.16 | intron | MFSD6 | M |
| 5199 | chr8 | 98789994 | 98790368 | Smooth Muscle cells | 0.66 | 0.19 | intron | LAPTM4B | M |
| 5200 | chr4 | 105411438 | 105411757 | Smooth Muscle cells | 0.62 | 0.19 | intron | CXXC4 | M |
| 5201 | chr2 | 2204068601 | 220407000 | Smooth Muscle cells | 0.63 | 0.21 | intron | CHPF | M |
| 5202 | chr6 | 32064206 | 32064259 | Smooth Muscle cells | 0.64 | 0.27 | intron | TNXB | M |
| 5203 | chr14 | 104312591 | 104312825 | Smooth Muscle cells | 0.57 | 0.21 | intron | PPP1R13B | M |
| 5204 | chr6 | 32064082 | 32064120 | Smooth Muscle cells | 0.64 | 0.3 | intron | TNXB | M |
| 5205 | chr2 | 74776766 | 74776868 | Smooth Muscle cells | 0.68 | 0.14 | intron | LOXL3 | M |
| 5206 | chr8 | 72916545 | 72916780 | Smooth Muscle cells | 0.69 | 0.21 | intron | LOC100132891 | M |
| 5207 | chr8 | 72758068 | 72758208 | Smooth Muscle cells | 0.66 | 0.21 | intron | LOC100132891 | M |
| 5208 | chr20 | 3653350 | 3653771 | Smooth Muscle cells | 0.73 | 0.11 | intron, exon | ADAM33, ADAM33 | M |
| 5209 | chr19 | 49939989 | 49940086 | Smooth Muscle cells | 0.74 | 0.21 | exon, intron | SLC17A7, SLC17A7 | M |
| 5210 | chr16 | 85206120 | 85206443 | Smooth Muscle cells | 0.71 | 0.19 | Intergenic | LOC400548 | M |
| 5211 | chr20 | 3653926 | 3654003 | Smooth Muscle cells | 0.73 | 0.22 | intron, exon | ADAM33, ADAM33 | M |
| 5212 | chr13 | 28000309 | 28000558 | Smooth Muscle cells | 0.62 | 0.14 | intron | GTF3A | M |
| 5213 | chr13 | 33589940 | 33590003 | Smooth Muscle cells | 0.66 | 0.19 | promoter-TSS | KL | M |
| 5214 | chr6 | 134175377 | 134175508 | Smooth Muscle cells | 0.61 | 0.14 | TTS | MGC34034 | M |
| 5215 | chr19 | 10077107 | 10077444 | Smooth Muscle cells | 0.67 | 0.22 | exon, intron | COL5A3, COL5A3 | M |
| 5216 | chr9 | 71735533 | 71735995 | Smooth Muscle cells | 0.56 | 0.12 | promoter-TSS | TIP2 | M |
| 5217 | chr5 | 77841405 | 77841623 | Thyroid Epithelium | 0.03 | 0.95 | intron | LHFPL2 | U |
| 5218 | chr12 | 111696042 | 111696091 | Thyroid Epithelium | 0.04 | 0.95 | Intron | CUX2 | U |
| 5219 | chr2 | 204339659 | 204339856 | Thyroid Epithelium | 0.04 | 0.95 | intron | RAPH1 | U |
| 5220 | chr6 | 1552781183 | 155278597 | Thyroid Epithelium | 0.03 | 0.93 | Intergenic | TIAM2 | U |
| 5221 | chr17 | 26526466 | 26526640 | Thyroid Epithelium | 0.05 | 0.95 | Intergenic | PYY2 | U |
| 5222 | chr17 | 312918363 | 31291913 | Thyroid Epithelium | 0.05 | 0.94 | Intergenic | SPACA3 | U |
| 5223 | chr12 | 132468369 | 132468606 | Thyroid Epithelium | 0.05 | 0.94 | intron | EP400 | U |
| 5224 | chr3 | 196643735 | 196643991 | Thyroid Epithelium | 0.08 | 0.96 | intron | SENP5 | U |
| 5225 | chr11 | 64524521 | 64524614 | Thyroid Epithelium | 0.07 | 0.94 | intron | PYGM | U |
| 5226 | chr6 | 52807841 | 52808039 | Thyroid Epithelium | 0.08 | 0.93 | Intergenic | GSTA3 | U |
| 5227 | chr5 | 153692596 | 153692856 | Thyroid Epithelium | 0.07 | 0.92 | intron | GALNT10 | U |
| 5228 | chr1 | 226442408 | 226442835 | Thyroid Epithelium | 0.1 | 0.95 | intron | LIN9 | U |
| 5229 | chr1 | 151742357 | 151742418 | Thyroid Epithelium | 0.11 | 0.95 | intron | OAZ3 | U |
| 5230 | chr18 | 60492252 | 60492411 | Thyroid Epithelium | 0.13 | 0.94 | intron | PHLPP1 | U |
| 5231 | chr16 | 87460982 | 87461122 | Thyroid Epithelium | 0.02 | 0.94 | intron | ZCCHC14 | U |
| 5232 | chr12 | 64410229 | 64410506 | Thyroid Epithelium | 0.03 | 0.94 | intron | SRGAPI | U |
| 5233 | chr7 | 110671964 | 110672287 | Thyroid Epithelium | 0.05 | 0.94 | intron | IMMP2L | U |
| 5234 | chr11 | 86500271 | 86500689 | Thyroid Epithelium | 0.03 | 0.91 | Intergenic | PRSS23 | U |
| 5235 | chr1 | 10441602 | 10442072 | Thyroid Epithelium | 0.04 | 0.92 | TTS | KIF1B | U |
| 5236 | chr9 | 132811819 | 132811955 | Thyroid Epithelium | 0.05 | 0.92 | Intergenic | GPR107 | U |
| 5237 | chr10 | 79598984 | 79599214 | Thyroid Epithelium | 0.06 | 0.93 | intron | DLG5 | U |
| 5238 | chr2 | 113489730 | 113490097 | Thyroid Epithelium | 0.02 | 0.89 | Intergenic | CKAP2L | U |
| 5239 | chr8 | 16642556 | 16642729 | Thyroid Epithelium | 0.06 | 0.92 | Intergenic | FGF20 | U |
| 5240 | chr2 | 26854285 | 26854460 | Thyroid Epithelium | 0.04 | 0.9 | intron | CIB4 | U |
| 5241 | chr7 | 68364312 | 68364488 | Thyroid Epithelium | 0.01 | 0.87 | Intergenic | AUTS2 | U |
| 5242 | chr1 | 9373886 | 9374190 | Thyroid Epithelium | 0.07 | 0.93 | intron | SPSB1 | U |
| 5243 | chr8 | 133881917 | 133882091 | Thyroid Epithelium | 0.02 | 0.87 | exon | TG | U |
| 5244 | chr10 | 78833441 | 78833640 | Thyroid Epithelium | 0.05 | 0.9 | intron | KCNMA1 | U |
| 5245 | chr2 | 9471561 | 9471829 | Thyroid Epithelium | 0.06 | 0.91 | intron | ASAP2 | U |
| 5246 | chr9 | 138170740 | 138170805 | Thyroid Epithelium | 0.06 | 0.9 | Intergenic | C9orf62 | U |
| 5247 | chr1 | 18058726 | 18058865 | Thyroid Epithelium | 0.08 | 0.92 | Intergenic | ACTL8 | U |
| 5248 | chr1 | 55372143 | 55372285 | Thyroid Epithelium | 0.06 | 0.9 | Intergenic | DHCR24 | U |
| 5249 | chr9 | 138073725 | 138073927 | Thyroid Epithelium | 0.05 | 0.89 | exon | LOC401557 | U |
| 5250 | chr13 | 53475567 | 53475831 | Thyroid Epithelium | 0.02 | 0.86 | Intergenic | PCDH8 | U |
| 5251 | chr17 | 2944952 | 2945112 | Thyroid Epithelium | 0.03 | 0.86 | Intergenic | OR1D5 | U |
| 5252 | chr15 | 58701798 | 58702038 | Thyroid Epithelium | 0.08 | 0.91 | Intergenic | LIPC | U |
| 5253 | chr1 | 113270906 | 113271168 | Thyroid Epithelium | 0.06 | 0.89 | TTS, Intergenic | FAM19A3, FAM19A3 | U |
| 5254 | chr3 | 184270869 | 184271026 | Thyroid Epithelium | 0.08 | 0.9 | Intergenic | EPHB3 | U |
| 5255 | chr10 | 131658341 | 131658561 | Thyroid Epithelium | 0.07 | 0.89 | intron | EBF3 | U |
| 5256 | chr3 | 42085691 | 42085906 | Thyroid Epithelium | 0.13 | 0.94 | Intergenic | ULK4 | U |
| 5257 | chr17 | 17737018 | 17737272 | Thyroid Epithelium | 0.11 | 0.92 | intron | SREBF1 | U |
| 5258 | chr7 | 1184393 | 1184562 | Thyroid Epithelium | 0.08 | 0.88 | Intergenic | C7orf50 | U |
| 5259 | chr8 | 101059069 | 101059333 | Thyroid Epithelium | 0.06 | 0.86 | intron | RGS22 | U |
| 5260 | chr14 | 97398487 | 97398775 | Thyroid Epithelium | 0.1 | 0.9 | Intergenic | VRK1 | U |
| 5261 | chr10 | 108227291 | 108227365 | Thyroid Epithelium | 0.09 | 0.88 | Intergenic | SORCS1 | U |
| 5262 | chr12 | 1238564801 | 123856657 | Thyroid Epithelium | 0.14 | 0.93 | Intergenic | SETDB | U |
| 5263 | chr2 | 86257018 | 86257382 | Thyroid Epithelium | 0.14 | 0.93 | intron | POLR1A | U |
| 5264 | chr5 | 60620150 | 60620521 | Thyroid Epithelium | 0.16 | 0.95 | Intergenic | ZSWIM6 | U |
| 5265 | chr17 | 38067990 | 38068365 | Thyroid Epithelium | 0.17 | 0.94 | intron | GSDMB | U |
| 5266 | chr8 | 28762393 | 28762890 | Thyroid Epithelium | 0.16 | 0.93 | intron | HMBOX1 | U |
| 5267 | chr19 | 14157259 | 14157464 | Thyroid Epithelium | 0.14 | 0.9 | intron | IL27RA | U |
| 5268 | chr5 | 179103360 | 179103599 | Thyroid Epithelium | 0.12 | 0.88 | Intergenic | CBY3 | U |
| 5269 | chr3 | 183408133 | 183408457 | Thyroid Epithelium | 0.18 | 0.94 | Intergenic | YEATS2 | U |
| 5270 | chr3 | 183972308 | 183972418 | Thyroid Epithelium | 0.13 | 0.88 | intron | ECE2 | U |
| 5271 | chr10 | 123691016 | 123691439 | Thyroid Epithelium | 0.16 | 0.91 | Intergenic | ATE1 | U |
| 5272 | chr15 | 75768694 | 75768952 | Thyroid Epithelium | 0.22 | 0.96 | intron | PTPN9 | U |
| 5273 | chr16 | 73058458 | 73058766 | Thyroid Epithelium | 0.19 | 0.93 | intron | ZFHX3 | U |
| 5274 | chr7 | 75953823 | 75954271 | Thyroid Epithelium | 0.2 | 0.94 | Intergenic | HSPB1 | U |
| 5275 | chr1 | 45581529 | 45581783 | Thyroid Epithelium | 0.21 | 0.94 | intron | ZSWIM5 | U |
| 5276 | chr16 | 19067687 | 19067891 | Thyroid Epithelium | 0.14 | 0.86 | intron | TMC7 | U |
| 5277 | chr6 | 126254856 | 126255172 | Thyroid Epithelium | 0.19 | 0.91 | Intergenic | HINT3 | U |
| 5278 | chr8 | 144617767 | 144618256 | Thyroid Epithelium | 0.22 | 0.93 | exon | ZC3H3 | U |
| 5279 | chr1 | 193160548 | 193160768 | Thyroid Epithelium | 0.26 | 0.96 | intron | CDC73 | U |
| 5280 | chr15 | 89011962 | 89012198 | Thyroid Epithelium | 0.24 | 0.94 | intron | MRPS11 | U |
| 5281 | chr17 | 1318285 | 1318632 | Thyroid Epithelium | 0.24 | 0.93 | Intergenic | YWHAE | U |
| 5282 | chr16 | 30026699 | 30026730 | Thyroid Epithelium | 0.27 | 0.94 | Intergenic | DOC2A | U |
| 5283 | chr19 | 5106165 | 5106472 | Thyroid Epithelium | 0.32 | 0.94 | intron | KDM4B | U |
| 5284 | chr15 | 41363111 | 41363492 | Thyroid Epithelium | 0.33 | 0.95 | intron | INO80 | U |
| 5285 | chr14 | 103375499 | 103375946 | Thyroid Epithelium | 0.19 | 0.93 | exon | TRAF3 | U |
| 5286 | chr10 | 134559550 | 134559866 | Thyroid Epithelium | 0.02 | 0.93 | intron | INPP5A | U |
| 5287 | chr2 | 3456139 | 3456394 | Thyroid Epithelium | 0.04 | 0.93 | intron | TRAPPC12 | U |
| 5288 | chr19 | 4676153 | 4676437 | Thyroid Epithelium | 0.04 | 0.93 | exon | DPP9 | U |
| 5289 | chr9 | 126368280 | 126368708 | Thyroid Epithelium | 0.04 | 0.92 | intron | DENND1A | U |
| 5290 | chr22 | 31522407 | 31522467 | Thyroid Epithelium | 0.08 | 0.94 | exon | INPP5J | U |
| 5291 | chr9 | 128200029 | 128200202 | Thyroid Epithelium | 0.03 | 0.89 | exon | MAPKAP1 | U |
| 5292 | chr19 | 46307986 | 46308452 | Thyroid Epithelium | 0.02 | 0.88 | exon, intron | RSPH6A | U |
| 5293 | chr6 | 139880341 | 13988092 | Thyroid Epithelium | 0.08 | 0.93 | Intergenic | RNF182 | U |
| 5294 | chr8 | 96038416 | 96038674 | Thyroid Epithelium | 0.08 | 0.92 | intron | NDUFAF6 | U |
| 5295 | chr1 | 1491550 | 1491950 | Thyroid Epithelium | 0.1 | 0.93 | intron | SSU72 | U |
| 5296 | chr14 | 21559955 | 21560302 | Thyroid Epithelium | 0.11 | 0.93 | exon | ZNF219 | U |
| 5297 | chr2 | 3449848 | 3450048 | Thyroid Epithelium | 0.05 | 0.95 | intron | TRAPPC12 | U |
| 5298 | chr1 | 1492055 | 1492375 | Thyroid Epithelium | 0.02 | 0.92 | Intron | SSU72 | U |
| 5299 | chr11 | 63562698 | 63562835 | Thyroid Epithelium | 0.04 | 0.93 | Intergenic | C11orf84 | U |
| 5300 | chr13 | 115052291 | 115052573 | Thyroid Epithelium | 0.03 | 0.92 | intron | UPF3A | U |
| 5301 | chr16 | 27509934 | 27510352 | Thyroid Epithelium | 0.03 | 0.92 | intron | GTF3C1 | U |
| 5302 | chr17 | 7076025 | 7076444 | Thyroid Epithelium | 0.02 | 0.91 | TTS | ASGR1 | U |
| 5303 | chr6 | 159044831 | 159045276 | Thyroid Epithelium | 0.04 | 0.93 | intron | TMEM181 | U |
| 5304 | chr2 | 236897052 | 236897538 | Thyroid Epithelium | 0.04 | 0.93 | intron | AGAP1 | U |
| 5305 | chr20 | 23969053 | 23969266 | Thyroid Epithelium | 0.04 | 0.92 | intron, exon | GGTLC1, GGTLC1 | U |
| 5306 | chr2 | 3266591 | 3267010 | Thyroid Epithelium | 0.07 | 0.95 | intron | TSSC1 | U |
| 5307 | chr20 | 9393796 | 9393983 | Thyroid Epithelium | 0.05 | 0.91 | intron | PLC84 | U |
| 5308 | chr16 | 16018823 | 16019143 | Thyroid Epithelium | 0.05 | 0.91 | Intergenic | ABCC1 | U |
| 5309 | chr19 | 30129869 | 30130034 | Thyroid Epithelium | 0.02 | 0.87 | Intergenic | PLEKHF1 | U |
| 5310 | chr13 | 22786818 | 22786991 | Thyroid Epithelium | 0.06 | 0.91 | Intergenic | LINC00424 | U |
| 5311 | chr14 | 21969885 | 21970111 | Thyroid Epithelium | 0.07 | 0.92 | intron | METTL3 | U |
| 5312 | chr2 | 130919013 | 130919124 | Thyroid Epithelium | 0.02 | 0.86 | intron | SMPD4 | U |
| 5313 | chr19 | 12701924 | 12702204 | Thyroid Epithelium | 0.09 | 0.93 | intron | ZNF490 | U |
| 5314 | chr2 | 208331846 | 208332302 | Thyroid Epithelium | 0.07 | 0.91 | Intergenic | CREB1 | U |
| 5315 | chr19 | 4676417 | 4676565 | Thyroid Epithelium | 0.03 | 0.86 | exon | DPP9 | U |
| 5316 | chr2 | 74848024 | 74848231 | Thyroid Epithelium | 0.09 | 0.92 | intron | M1AP | U |
| 5317 | chr1 | 154931253 | 154931637 | Thyroid Epithelium | 0.04 | 0.87 | exon | PYGO2 | U |
| 5318 | chr20 | 62242039 | 62242246 | Thyroid Epithelium | 0.04 | 0.86 | intron | GMEB2 | U |
| 5319 | chr2 | 240298350 | 240298596 | Thyroid Epithelium | 0.08 | 0.9 | intron | HDAC4 | U |
| 5320 | chr14 | 58194229 | 68194554 | Thyroid Epithelium | 0.11 | 0.93 | intron | RDH12 | U |
| 5321 | chr1 | 57046407 | 57046901 | Thyroid Epithelium | 0.1 | 0.92 | Intergenic | PPAP2B | U |
| 5322 | chr13 | 113488301 | 113488591 | Thyroid Epithelium | 0.1 | 0.91 | intron | ATP11A | U |
| 5323 | chr2 | 206605235 | 206605573 | Thyroid Epithelium | 0.11 | 0.92 | exon, intron, intron | NRP2, NRP2, NRP2 | U |
| 5324 | chr9 | 1004311281 | 100431526 | Thyroid Epithelium | 0.09 | 0.9 | intron | NCBP1 | U |
| 5325 | chr16 | 1676814 | 1677233 | Thyroid Epithelium | 0.13 | 0.94 | intron | CRAMP1L | U |
| 5326 | chr15 | 65241739 | 65242202 | Thyroid Epithelium | 0.13 | 0.93 | intron | ANKDD1A | U |
| 5327 | chr14 | 33392213 | 33392681 | Thyroid Epithelium | 0.12 | 0.92 | Intergenic | NPAS3 | U |
| 5328 | chr3 | 195709678 | 195709900 | Thyroid Epithelium | 0.16 | 0.95 | intron | SDHAP1 | U |
| 5329 | chr21 | 44441320 | 44441550 | Thyroid Epithelium | 0.11 | 0.9 | exon, intron, intron | PKNOX1, PKNOX1, PKN [symbol] | U |
| 5330 | chr2 | 60986229 | 60986557 | Thyroid Epithelium | 0.16 | 0.95 | intron | PAPOLG | U |
| 5331 | chr14 | 73391680 | 73391869 | Thyroid Epithelium | 0.16 | 0.93 | Intergenic | DCAF4 | U |
| 5332 | chr20 | 54330073 | 5433358 | Thyroid Epithelium | 0.15 | 0.92 | Intergenic | LINC00658 | U |
| 5333 | chr9 | 139134458 | 139134679 | Thyroid Epithelium | 0.1 | 0.86 | intron | QSOX2 | U |
| 5334 | chr12 | 121756173 | 121756406 | Thyroid Epithelium | 0.19 | 0.94 | intron | ANAPC5 | U |
| 5335 | chr20 | 23968631 | 23968979 | Thyroid Epithelium | 0.16 | 0.91 | promoter-TSS, intro [symbol] | GGTLC1, GGTLC1 | U |
| 5336 | chr20 | 33903471 | 33903868 | Thyroid Epithelium | 0.18 | 0.93 | intron | UQCC | U |
| 5337 | chr22 | 31522899 | 31523087 | Thyroid Epithelium | 0.17 | 0.91 | intron | INPP5J | U |
| 5338 | chr11 | 72146423 | 72146633 | Thyroid Epithelium | 0.19 | 0.91 | promoter-TSS | CLPB | U |
| 5339 | chr2 | 231530504 | 231530900 | Thyroid Epithelium | 0.19 | 0.91 | Intergeni | LOC151475 | U |
| 5340 | chr6 | 3070673 | 3070765 | Thyroid Epithelium | 0.23 | 0.94 | Intergenic | RIPK1 | U |
| 5341 | chr4 | 1738884 | 1739194 | Thyroid Epithelium | 0.27 | 0.96 | exon | TACC3 | U |
| 5342 | chr15 | 91268331 | 91268748 | Thyroid Epithelium | 0.28 | 0.95 | intron | BLM | U |
| 5343 | chr9 | 127120341 | 127120762 | Thyroid Epithelium | 0.25 | 0.92 | exon | LOC100129034 | U |
| 5344 | chr13 | 109567908 | 109568303 | Thyroid Epithelium | 0.87 | 0.14 | intron | MYO16 | M |
| 5345 | chr4 | 140476076 | 140476258 | Thyroid Epithelium | 0.75 | 0.06 | intron | SETD7 | M |
| 5346 | chr3 | 150479084 | 150479479 | Thyroid Epithelium | 0.79 | 0.13 | intron | SIAH2 | M |
| 5347 | chr11 | 113648895 | 113649142 | Thyroid Epithelium | 0.76 | 0.11 | Intergenic | ICLDN25 | M |
| 5348 | chr4 | 186316500 | 186316842 | Thyroid Epithelium | 0.72 | 0.07 | Intergenic | ANKRD37 | M |
| 5349 | chr2 | 64370060 | 64370441 | Thyroid Epithelium | 0.71 | 0.06 | intron | PELI1 | M |
| 5350 | chr10 | 7527240 | 7527385 | Thyroid Epithelium | 0.82 | 0.18 | Intergenic | SFMBT2 | M |
| 5351 | chr19 | 1258902 | 1259109 | Thyroid Epithelium | 0.8 | 0.17 | exon, TTS | MIDN, MIDN | M |
| 5352 | chr6 | 128839793 | 128840033 | Thyroid Epithelium | 0.74 | 0.12 | intron | PTPRK | M |
| 5353 | chr15 | 96883151 | 96883546 | Thyroid Epithelium | 0.7 | 0.08 | exon | NR2F2 | M |
| 5354 | chr14 | 81462751 | 81463004 | Thyroid Epithelium | 0.67 | 0.06 | intron | TSHR | M |
| 5355 | chr1 | 1493336411 | 149333733 | Thyroid Epithelium | 0.68 | 0.08 | Intergenic | FCGR1C | M |
| 5356 | chr1 | 1493337401 | 149333805 | Thyroid Epithelium | 0.68 | 0.12 | Intergenic | FCGR1C | M |
| 5357 | chr14 | 90865037 | 90865362 | Thyroid Epithelium | 0.61 | 0.05 | intron | CALM1 | M |
| 5358 | chr11 | 128377113 | 128377421 | Thyroid Epithelium | 0.58 | 0.04 | intron | ETS1 | M |
| 5359 | chr10 | 8102090 | 8102352 | Thyroid Epithelium | 0.88 | 0.15 | intron | GATA3 | M |
| 5360 | chr11 | 57244279 | 57244587 | Thyroid Epithelium | 0.86 | 0.08 | TTS | RTN4RL2 | M |
| 5361 | chr10 | 88023129 | 88023186 | Thyroid Epithelium | 0.93 | 0.17 | intron | GRID1 | M |
| 5362 | chr11 | 46582020 | 46582513 | Thyroid Epithelium | 0.75 | 0.04 | intron | AMBRA1 | M |
| 5363 | chr9 | 1005038723 | 100504016 | Thyroid Epithelium | 0.8 | 0.12 | Intergenic | EXPA | M |
| 5364 | chr13 | 459911221 | 45991446 | Thyroid Epithelium | 0.81 | 0.13 | intron | SLC25A30 | M |
| 5365 | chr3 | 44039086 | 44039290 | Thyroid Epithelium | 0.75 | 0.08 | Intergenic | MIR138-1 | M |
| 5366 | chr7 | 150784966 | 150785108 | Thyroid Epithelium | 0.61 | 0.06 | exon, intron | AGAP3, AGAP3 | M |
| 5367 | chr14 | 75413103 | 75413361 | Thyroid Epithelium | 0.6 | 0.06 | intron | PGF | M |
| 5368 | chr8 | 37595216 | 37595411 | Thyroid Epithelium | 0.57 | 0.06 | intron | ERLIN2 | M |
| 5369 | chr7 | 134849833 | 134850162 | Adipocytes | 0.17 | 0.83 | TTS | C7orf49 | U |
| 5370 | chr11 | 27502491 | 27502645 | Adipocytes | 0.29 | 0.92 | Intergenic | LGR4 | U |
| 5371 | chr11 | 62304487 | 62304526 | Adipocytes | 0.27 | 0.9 | intron | AHNAK | U |
| 5372 | chr1 | 110426640 | 110427129 | Adipocytes | 0.3 | 0.9 | Intergenic | CSF1 | U |
| 5373 | chr12 | 1095953711 | 109595608 | Adipocytes | 0.27 | 0.86 | intron | ACACB | U |
| 5374 | chr5 | 79945953 | 79946334 | Adipocytes | 0.25 | 0.84 | exon | MTRNR2L2 | U |
| 5375 | chr5 | 9388792 | 99388843 | Adipocytes | 0.22 | 0.8 | Intergenic | LOC100133050 | U |
| 5376 | chr11 | 75537042 | 75537263 | Adipocytes | 0.3 | 0.88 | intron | UVRAG | U |
| 5377 | chr6 | 155357994 | 155358379 | Adipocytes | 0.3 | 0.88 | Intergenic | TIAM2 | U |
| 5378 | chr5 | 99388999 | 99389072 | Adipocytes | 0.24 | 0.82 | Intergenic | LOC100133050 | U |
| 5379 | chr17 | 47692960 | 47693459 | Adipocytes | 0.29 | 0.85 | intron | SPOP | U |
| 5380 | chr19 | 5714972 | 5715185 | Adipocytes | 0.33 | 0.88 | intron | LONP1 | U |
| 5381 | chr10 | 71232978 | 71233197 | Adipocytes | 0.35 | 0.9 | intron | TSPAN15 | U |
| 5382 | chr12 | 125111263 | 125111660 | Adipocytes | 0.34 | 0.89 | Intergenic | NCOR2 | U |
| 5383 | chr6 | 62284239 | 62284318 | Adipocytes | 0.33 | 0.87 | Intergenic | KHDRBS2 | U |
| 5384 | chr7 | 95237088 | 95237447 | Adipocytes | 0.31 | 0.85 | Intergenic | PDK4 | U |
| 5385 | chr5 | 134261080 | 134261365 | Adipocytes | 0.3 | 0.83 | intron | PCBD2 | U |
| 5386 | chr15 | 73988788 | 73988963 | Adipocytes | 0.35 | 0.87 | intron | CD276 | U |
| 5387 | chr11 | 93266034 | 93266360 | Adipocytes | 0.32 | 0.84 | intron | C11orf75 | U |
| 5388 | chr17 | 80024096 | 80024157 | Adipocytes | 0.33 | 0.84 | promoter-TSS | DUS1L | U |
| 5389 | chr18 | 71452236 | 71452389 | Adipocytes | 0.32 | 0.83 | Intergenic | FBXO15 | U |
| 5390 | chr2 | 127878592 | 127878832 | Adipocytes | 0.22 | 0.88 | Intergenic | BIN1 | U |
| 5391 | chr17 | 51183100 | 51183164 | Adipocytes | 0.18 | 0.81 | Intergenic | C17orf112 | U |
| 5392 | chr3 | 1793882113 | 179388365 | Adipocytes | 0.25 | 0.86 | intron | USP13 | U |
| 5393 | chr11 | 10530619 | 10530845 | Adipocytes | 0.21 | 0.82 | promoter-TSS | MTRNR2L8 | U |
| 5394 | chr5 | 134259017 | 134259287 | Adipocytes | 0.19 | 0.8 | intron | PCBD2 | U |
| 5395 | chr17 | 41004636 | 41004930 | Adipocytes | 0.27 | 0.88 | exon | AOC3 | U |
| 5396 | chr5 | 79946939 | 79947087 | Adipocytes | 0.2 | 0.8 | promoter-TSS | MTRNR2L2 | U |
| 5397 | chr17 | 1378763 | 1378921 | Adipocytes | 0.31 | 0.91 | intron | MYO1C | U |
| 5398 | chr12 | 109595409 | 109595609 | Adipocytes | 0.24 | 0.83 | intron | ACACB | U |
| 5399 | chr17 | 51183274 | 51183341 | Adipocytes | 0.18 | 0.76 | Intergenic | C17orf112 | U |
| 5400 | chr17 | 41004345 | 41004548 | Adipocytes | 0.27 | 0.85 | exon | AOC3 | U |
| 5401 | chr12 | 115682865 | 115683070 | Adipocytes | 0.28 | 0.86 | Intergenic | TBX3 | U |
| 5402 | chr9 | 133811283 | 133811331 | Adipocytes | 0.29 | 0.86 | intron | FIBCD1 | U |
| 5403 | chr9 | 130660439 | 130660604 | Adipocytes | 0.25 | 0.82 | intron | ST6GALNAC6 | U |
| 5404 | chr13 | 1071152608 | 107115475 | Adipocytes | 0.28 | 0.85 | Intergenic | EFNB2 | U |
| 5405 | chr10 | 71891102 | 71891451 | Adipocytes | 0.28 | 0.85 | intron | AIFM2 | U |
| 5406 | chr11 | 10530073 | 10530501 | Adipocytes | 0.23 | 0.8 | promoter-TSS | MIR4485 | U |
| 5407 | chr19 | 48219990 | 48220176 | Adipocytes | 0.28 | 0.84 | exon | EHD2 | U |
| 5408 | chr13 | 110896068 | 110896287 | Adipocytes | 0.31 | 0.87 | intron | COL4A1 | U |
| 5409 | chr12 | 117088493 | 117088772 | Adipocytes | 0.28 | 0.84 | Intergenic | C12orf49 | U |
| 5410 | chr2 | 10955156 | 10955471 | Adipocytes | 0.32 | 0.88 | Intergenic | PDIA6 | U |
| 5411 | chr17 | 40577387 | 40577762 | Adipocytes | 0.24 | 0.8 | Intergenic | PTRF | U |
| 5412 | chr21 | 35171461 | 35171860 | Adipocytes | 0.33 | 0.89 | intron | ITSN1 | U |
| 5413 | chr8 | 23244277 | 23244404 | Adipocytes | 0.34 | 0.89 | intron | LOXL2 | U |
| 5414 | chr5 | 99384926 | 99385065 | Adipocytes | 0.21 | 0.76 | Intergenic | LOC100133050 | U |
| 5415 | chr5 | 134262993 | 134263324 | Adipocytes | 0.28 | 0.83 | promoter-TSS | MIR4461 | U |
| 5416 | chr9 | 129882759 | 129883094 | Adipocytes | 0.31 | 0.86 | intron | ANGPTL2 | U |
| 5417 | chr2 | 242517608 | 242518105 | Adipocytes | 0.33 | 0.87 | Intergenic | BOK-AS1 | U |
| 5418 | chr12 | 1903818 | 1903870 | Adipocytes | 0.36 | 0.89 | intron | CACNA2D4 | U |
| 5419 | chr8 | 10576848 | 10576922 | Adipocytes | 0.35 | 0.88 | Intergenic | SOX7 | U |
| 5420 | chr17 | 62011700 | 62011798 | Adipocytes | 0.27 | 0.8 | Intergenic | CD79B | U |
| 5421 | chr11 | 18994018 | 18994128 | Adipocytes | 0.32 | 0.85 | Intergenic | MRGPRX1 | U |
| 5422 | chr5 | 99386958 | 99387077 | Adipocytes | 0.25 | 0.78 | Intergenic | LOC100133050 | U |
| 5423 | chr7 | 1096422 | 1096548 | Adipocytes | 0.3 | 0.83 | promoter-TSS | GPR146 | U |
| 5424 | chr4 | 144203887 | 144204237 | Adipocytes | 0.34 | 0.87 | Intergenic | GAB1 | U |
| 5425 | chr5 | 99386389 | 99386813 | Adipocytes | 0.23 | 0.76 | Intergenic | LOC100133050 | U |
| 5426 | chr5 | 93905847 | 93906089 | Adipocytes | 0.25 | 0.77 | intron | KIAA0825 | U |
| 5427 | chr6 | 1518572563 | 151857511 | Adipocytes | 0.35 | 0.87 | intron | CCDC170 | U |
| 5428 | chr2 | 44463142 | 44463413 | Adipocytes | 0.33 | 0.85 | Intergenic | SLC3A1 | U |
| 5429 | chr12 | 125114610 | 125114926 | Adipocytes | 0.35 | 0.87 | Intergenic | NCOR2 | U |
| 5430 | chr20 | 60782586 | 60782684 | Adipocytes | 0.3 | 0.81 | Intergenic | HRH3 | U |
| 5431 | chr2 | 109942715 | 109942956 | Adipocytes | 0.34 | 0.85 | intron | GH3RF3 | U |
| 5432 | chr12 | 94389336 | 94389633 | Adipocytes | 0.32 | 0.83 | Intergenic | PLXNC1 | U |
| 5433 | chr2 | 17182671 | 1718569 | Adipocytes | 0.33 | 0.84 | intron | PXDN | U |
| 5434 | chr10 | 4674820 | 4675298 | Adipocytes | 0.34 | 0.85 | Intergenic | LINC00705 | U |
| 5435 | chr12 | 2037717 | 2037873 | Adipocytes | 0.35 | 0.85 | TTS | LOC100271702 | U |
| 5436 | chr14 | 95948245 | 95948497 | Adipocytes | 0.35 | 0.85 | Intergenic | SYNE3 | U |
| 5437 | chr8 | 99009173 | 99009498 | Adipocytes | 0.37 | 0.87 | intron | MATN2 | U |
| 5438 | chr16 | 48565470 | 48565564 | Adipocytes | 0.37 | 0.86 | Intergenic | N4BP1 | U |
| 5439 | chr1 | 96991151 | 96991596 | Adipocytes | 0.36 | 0.85 | Intergenic | PTBP2 | U |
| 5440 | chr4 | 8188123 | 8188193 | Adipocytes | 0.35 | 0.83 | Intergenic | SH3TC1 | U |
| 5441 | chr5 | 178296635 | 178296851 | Adipocytes | 0.36 | 0.84 | intron | ZNF354B | U |
| 5442 | chr8 | 62322984 | 62323287 | Adipocytes | 0.36 | 0.83 | intron | CLVS1 | U |
| 5443 | chr20 | 599201551 | 59920394 | Adipocytes | 0.36 | 0.82 | intron | CDH4 | U |
| 5444 | chr17 | 41722916 | 41723060 | Adipocytes | 0.36 | 0.79 | intron | MEOX1 | U |
| 5445 | chr14 | 90274328 | 90274554 | Adipocytes | 0.39 | 0.79 | intron | EFCAB11 | U |
| 5446 | chr19 | 57125150 | 57125480 | Adipocytes | 0.31 | 0.9 | intron | ZNF71 | U |
| 5447 | chr9 | 111944902 | 111945063 | Adipocytes | 0.35 | 0.83 | intron | EPB41L4B | U |
| 5448 | chr22 | 38051826 | 38052316 | Adipocytes | 0.36 | 0.82 | TTS, exon | SH3BP1 | U |
| 5449 | chr22 | 39664962 | 39665253 | Adipocytes | 0.39 | 0.83 | Intergenic | PDGFB | U |
| 5450 | chr11 | 10529463 | 10529717 | Adipocytes | 0.22 | 0.8 | TTS | AMPD3 | U |
| 5451 | chr5 | 99389410 | 99389822 | Adipocytes | 0.28 | 0.83 | Intergenic | LOC100133050 | U |
| 5452 | chr9 | 134195865 | 134195923 | Adipocytes | 0.35 | 0.89 | Intergenic | PPAPDC3 | U |
| 5453 | chr9 | 19129150 | 19129252 | Adipocytes | 0.3 | 0.82 | Intergenic | PLIN2 | U |
| 5454 | chr17 | 46693343 | 46693444 | Adipocytes | 0.64 | 0.14 | Intergenic | HOXB8 | M |
| 5455 | chr17 | 46694698 | 46694750 | Adipocytes | 0.63 | 0.16 | Intergenic | HOXB8 | M |
| 5456 | chr5 | 135313253 | 135313315 | Adipocytes | 0.61 | 0.16 | Intergenic | LECT2 | M |
| 5457 | chr17 | 46693078 | 45693229 | Adipocytes | 0.57 | 0.12 | promoter-TSS | HOXB8 | M |
| 5458 | chr8 | 98789994 | 98790368 | Adipocytes | 0.64 | 0.19 | intron | LAPTM4B | M |
| 5459 | chr17 | 46692963 | 46693003 | Adipocytes | 0.6 | 0.2 | promoter-TSS | HOXB8 | M |
| 5460 | chr4 | 139786227 | 139786583 | Adipocytes | 0.53 | 0.13 | Intergenic | CCRN4L | M |
| 5461 | chr17 | 46692823 | 46692893 | Adipocytes | 0.53 | 0.14 | promoter-TSS | HOXB8 | M |
| 5462 | chr10 | 6622900 | 6623212 | Adipocytes | 0.54 | 0.15 | promoter-TSS | PRKCQ | M |
| 5463 | chr9 | 138944784 | 138945185 | Adipocytes | 0.85 | 0.56 | intron | NACC2 | M |
| 5464 | chr7 | 27225772 | 27225898 | Adipocytes | 0.54 | 0.18 | promoter-TSS | HOXA11 | M |
| 5465 | chr14 | 62035107 | 62035599 | Adipocytes | 0.58 | 0.09 | Intergenic | FLI22447 | M |
| 5466 | chr11 | 850093 | 850248 | Adipocytes | 0.55 | 0.12 | intron | TSPAN4 | M |
| 5467 | chr6 | 85476296 | 85476447 | Adipocytes | 0.55 | 0.14 | Intergenic | TBX18 | M |
| 5468 | chr9 | 790752743 | 79075498 | Adipocytes | 0.53 | 0.15 | intron | GCNT1 | M |
| 5469 | chr11 | 44325473 | 44325888 | Adipocytes | 0.54 | 0.16 | intron | ALX4 | M |
| 5470 | chr14 | 95237828 | 95237939 | Adipocytes | 0.54 | 0.21 | Intergenic | GSC | M |
| 5471 | chr1 | 229476664 | 229476935 | Neuron CNS | 0.14 | 0.93 | intron | CCSAP | U |
| 5472 | chr2 | 210526620 | 210527068 | Neuron CNS | 0.15 | 0.91 | intron | MAP2 | U |
| 5473 | chr20 | 61812020 | 61812096 | Neuron CNS | 0.18 | 0.92 | Intergenic | MIR124-3 | U |
| 5474 | chr1 | 156388230 | 156388343 | Neuron CNS | 0.2 | 0.94 | intron | C1orf61 | U |
| 5475 | chr19 | 14314665 | 14314864 | Neuron CNS | 0.18 | 0.92 | intron | LPHN1 | U |
| 5476 | chr15 | 70699828 | 70699859 | Neuron CNS | 0.18 | 0.9 | Intergenic | TLE3 | U |
| 5477 | chr2 | 51254072 | 51254313 | Neuron CNS | 0.22 | 0.92 | intron | NRXN1 | U |
| 5478 | chr1 | 1510348693 | 151035128 | Neuron CNS | 0.29 | 0.95 | intron | MLLT11 | U |
| 5479 | chr17 | 431772141 | 43177273 | Neuron CNS | 0.31 | 0.96 | intron | NMT1 | U |
| 5480 | chr7 | 44427912 | 44428238 | Neuron CNS | 0.29 | 0.93 | intron | NUDCD3 | U |
| 5481 | chr16 | 15223252 | 15223657 | Neuron CNS | 0.29 | 0.93 | Intergenic | MIR3180-4 | U |
| 5482 | chr11 | 1301635 | 1301925 | Neuron CNS | 0.28 | 0.92 | intron | TOLLIP | U |
| 5483 | chr4 | 2417547 | 2417899 | Neuron CNS | 0.25 | 0.89 | intron | ZFYVE28 | U |
| 5484 | chr18 | 13374254 | 13374751 | Neuron CNS | 0.28 | 0.92 | intron | LDLRAD4 | U |
| 5485 | chr12 | 133306612 | 133307002 | Neuron CNS | 0.3 | 0.94 | exon | ANKLE2 | U |
| 5486 | chr5 | 145986293 | 145986487 | Neuron CNS | 0.3 | 0.91 | intron | PPP2R2B | U |
| 5487 | chr1 | 19517091 | 1951897 | Neuron CNS | 0.23 | 0.77 | intron | GABRD | U |
| 5488 | chr22 | 45343200 | 45343314 | Neuron CNS | 0.36 | 0.88 | intron | PHF21B | U |
| 5489 | chr11 | 63451294 | 63451377 | Neuron CNS | 0.15 | 0.93 | intron | RTN3 | U |
| 5490 | chr19 | 18313277 | 18313355 | Neuron CNS | 0.14 | 0.9 | intron, exon | RAB3A, RAB3A | U |
| 5491 | chr20 | 60638378 | 60638717 | Neuron CNS | 0.08 | 0.84 | intron | TAF4 | U |
| 5492 | chr2 | 39895425 | 39895781 | Neuron CNS | 0.15 | 0.91 | intron | TMEM178A | U |
| 5493 | chr16 | 11773368 | 11773555 | Neuron CNS | 0.15 | 0.9 | TTS | SNN | U |
| 5494 | chr2 | 512537281 | 51254055 | Neuron CNS | 0.12 | 0.86 | intron | NRXN1 | U |
| 5495 | chr11 | 7841361 | 784317 | Neuron CNS | 0.17 | 0.9 | Intergenic | NS3BP | U |
| 5496 | chr1 | 241518958 | 241519216 | Neuron CNS | 0.14 | 0.87 | exon | RGS7 | U |
| 5497 | chr10 | 11321389 | 11321464 | Neuron CNS | 0.2 | 0.91 | intron | CELF2 | U |
| 5498 | chr1 | 151684072 | 151684272 | Neuron CNS | 0.16 | 0.87 | intron | CELF3 | U |
| 5499 | chr1 | 9912213 | 9912434 | Neuron CNS | 0.16 | 0.86 | intron | CTNNBIP1 | U |
| 5500 | chr13 | 113747701 | 113747990 | Neuron CNS | 0.19 | 0.88 | intron | MCF2L | U |
| 5501 | chr11 | 123397344 | 123397706 | Neuron CNS | 0.18 | 0.87 | intron | GRAMD1B | U |
| 5502 | chr12 | 41087794 | 41088289 | Neuron CNS | 0.19 | 0.88 | intron | CNTN1 | U |
| 5503 | chr4 | 153023924 | 153024013 | Neuron CNS | 0.2 | 0.88 | Intergenic | PET112 | U |
| 5504 | chr14 | 105783388 | 105783537 | Neuron CNS | 0.19 | 0.87 | intron | PACS2 | U |
| 5505 | chr8 | 74679595 | 74679948 | Neuron CNS | 0.22 | 0.9 | Intergenic | STAU2 | U |
| 5506 | chr16 | 30428390 | 30428599 | Neuron CNS | 0.22 | 0.89 | intron | ZNF771 | U |
| 5507 | chr15 | 83377031 | 83377529 | Neuron CNS | 0.15 | 0.82 | intron | AP3B2 | U |
| 5508 | chr16 | 752506 | 752557 | Neuron CNS | 0.2 | 0.86 | intron | FBXL16 | U |
| 5509 | chr1 | 111144600 | 111144789 | Neuron CNS | 0.25 | 0.91 | exon | KCNA2 | U |
| 5510 | chr9 | 137981452 | 137981515 | Neuron CNS | 0.12 | 0.77 | Intron | OLFM1 | U |
| 5511 | chr12 | 19473611 | 1947431 | Neuron CNS | 0.25 | 0.9 | intron | CACNA2D4 | U |
| 5512 | chr14 | 102464020 | 102464276 | Neuron CNS | 0.22 | 0.87 | intron | DYNC1H1 | U |
| 5513 | chr3 | 184057683 | 184058084 | Neuron CNS | 0.22 | 0.87 | intron | FAM131A | U |
| 5514 | chr5 | 140903584 | 140904045 | Neuron CNS | 0.22 | 0.87 | intron | DIAPH1 | U |
| 5515 | chr16 | 752640 | 752702 | Neuron CNS | 0.25 | 0.89 | intron | FBXL16 | U |
| 5516 | chr11 | 64402890 | 64403123 | Neuron CNS | 0.25 | 0.89 | intron | NRXN2 | U |
| 5517 | chr11 | 117741663 | 117741992 | Neuron CNS | 0.23 | 0.87 | intron | FXYD6 | U |
| 5518 | chr11 | 123433332 | 123433827 | Neuron CNS | 0.26 | 0.9 | intron | GRAMD1B | U |
| 5519 | chr1 | 183388184 | 183388241 | Neuron CNS | 0.23 | 0.86 | promoter-TSS | NMNAT2 | U |
| 5520 | chr4 | 6344599 | 6344673 | Neuron CNS | 0.26 | 0.89 | intron | PPP2R2C | U |
| 5521 | chr11 | 64400262 | 64400339 | Neuron CNS | 0.28 | 0.91 | intron | NRXN2 | U |
| 5522 | chr16 | 255465 | 255750 | Neuron CNS | 0.26 | 0.89 | intron | LUC7L | U |
| 5523 | chr8 | 41751840 | 41752192 | Neuron CNS | 0.28 | 0.91 | intron | ANK1 | U |
| 5524 | chr2 | 26202681 | 26203068 | Neuron CNS | 0.28 | 0.91 | intron | KIF3C | U |
| 5525 | chr17 | 2712162 | 2712631 | Neuron CNS | 0.26 | 0.89 | intron | RAPIGAP2 | U |
| 5526 | chr19 | 58236184 | 58236661 | Neuron CNS | 0.31 | 0.94 | intron | ZNF671 | U |
| 5527 | chr9 | 138910662 | 138910708 | Neuron CNS | 0.23 | 0.85 | intron | NACC2 | U |
| 5528 | chr15 | 792025153 | 79202722 | Neuron CNS | 0.24 | 0.86 | Intergenic | CTSH | U |
| 5529 | chr2 | 85658853 | 85659138 | Neuron CNS | 0.23 | 0.85 | Intergenic | SH2D6 | U |
| 5530 | chr11 | 47605869 | 47606088 | Neuron CNS | 0.27 | 0.88 | exon | NDUFS3 | U |
| 5531 | chr1 | 209796880 | 209797298 | Neuron CNS | 0.3 | 0.91 | promoter-TSS | MIR4260 | U |
| 5532 | chr16 | 85452473 | 85452688 | Neuron CNS | 0.33 | 0.93 | Intergenic | MIR5093 | U |
| 5533 | chr7 | 44316367 | 44316847 | Neuron CNS | 0.3 | 0.9 | intron | CAMK2B | U |
| 5534 | chr17 | 29038739 | 29038972 | Neuron CNS | 0.35 | 0.94 | intron | SUZ12P1 | U |
| 5535 | chr7 | 150789017 | 150789270 | Neuron CNS | 0.32 | 0.91 | intron | AGAP3 | U |
| 5536 | chr12 | 1215923881 | 121592620 | Neuron CNS | 0.31 | 0.89 | intron | P2RX7 | U |
| 5537 | chr19 | 51045764 | 51046205 | Neuron CNS | 0.26 | 0.84 | intron | LRRC4B | U |
| 5538 | chr4 | 2357948 | 2358138 | Neuron CNS | 0.28 | 0.85 | intron | ZFYVE28 | U |
| 5539 | chr20 | 1900281 | 1900598 | Neuron CNS | 0.26 | 0.83 | intron | SIRPA | U |
| 5540 | chr3 | 13094209 | 13094541 | Neuron CNS | 0.32 | 0.89 | intron | IQSEC1 | U |
| 5541 | chr12 | 123425582 | 123425943 | Neuron CNS | 0.33 | 0.9 | intron | ABCBS | U |
| 5542 | chr9 | 140035658 | 140036053 | Neuron CNS | 0.17 | 0.74 | intron | GRIN1 | U |
| 5543 | chr17 | 56166789 | 56167198 | Neuron CNS | 0.32 | 0.89 | exon, TTS | DYNLL2, DYNLL2 | U |
| 5544 | chr9 | 141008005 | 141008490 | Neuron CNS | 0.29 | 0.86 | intron | CACNA1B | U |
| 5545 | chr19 | 501955401 | 50195619 | Neuron CNS | 0.26 | 0.82 | exon | CPT1C | U |
| 5546 | chr7 | 97668445 | 97668629 | Neuron CNS | 0.28 | 0.83 | Intergenic | OCM2 | U |
| 5547 | chr4 | 3531996 | 3532367 | Neuron CNS | 0.32 | 0.87 | intron | LRPAP1 | U |
| 5548 | chr9 | 137994537 | 137994881 | Neuron CNS | 0.31 | 0.85 | intron | OLFM1 | U |
| 5549 | chr15 | 31596905 | 31597283 | Neuron CNS | 0.36 | 0.9 | Intergenic | KLF13 | U |
| 5550 | chr2 | 241756926 | 241757324 | Neuron CNS | 0.33 | 0.87 | intron | KIF1A | U |
| 5551 | chr5 | 141263876 | 141263978 | Neuron CNS | 0.21 | 0.74 | Intergenic | PCDH1 | U |
| 5552 | chr7 | 71912358 | 71912501 | Neuron CNS | 0.22 | 0.75 | Intergenic | CALN1 | U |
| 5553 | chr4 | 951231 | 951461 | Neuron CNS | 0.33 | 0.86 | intron | TMEM175 | U |
| 5554 | chr9 | 140993938 | 140994227 | Neuron CNS | 0.38 | 0.91 | intron | CACNA1B | U |
| 5555 | chr10 | 125426324 | 125426383 | Neuron CNS | 0.25 | 0.76 | exon | GPR26 | U |
| 5556 | chr11 | 29077961 | 29078058 | Neuron CNS | 0.36 | 0.86 | intron | LOC646278 | U |
| 5557 | chr11 | 17786781 | 17786852 | Neuron CNS | 0.09 | 0.85 | intron | KCNC1 | U |
| 5558 | chr11 | 177610121 | 17761372 | Neuron CNS | 0.15 | 0.85 | intron | KCNC1 | U |
| 5559 | chr16 | 87728212 | 87728541 | Neuron CNS | 0.26 | 0.93 | intron | UPH3 | U |
| 5560 | chr7 | 127670469 | 127670634 | Neuron CNS | 0.08 | 0.93 | exon | LRRC4 | U |
| 5561 | chr7 | 5391292 | 5391419 | Neuron CNS | 0.21 | 0.94 | intron | TNRC18 | U |
| 5562 | chr7 | 1276701701 | 127670461 | Neuron CNS | 0.25 | 0.96 | exon | LRRCA | U |
| 5563 | chr2 | 241757494 | 241757743 | Neuron CNS | 0.12 | 0.82 | intron | KIF1A | U |
| 5564 | chr20 | 34997369 | 34997568 | Neuron CNS | 0.15 | 0.82 | intron | DLGAP4 | U |
| 5565 | chr22 | 26847942 | 26847959 | Neuron CNS | 0.27 | 0.89 | exon | HPS4 | U |
| 5566 | chr3 | 16371412 | 16371683 | Neuron CNS | 0.27 | 0.87 | intron | RFTN1 | U |
| 5567 | chr11 | 62474735 | 62475079 | Neuron CNS | 0.07 | 0.86 | promoter-TSS | GNG3 | U |
| 5568 | chr19 | 18313493 | 18313542 | Neuron CNS | 0.11 | 0.84 | exon | RAB3A | U |
| 5569 | chr14 | 69157866 | 69158048 | Neuron CNS | 0.17 | 0.89 | Intergenic | ZFP36L1 | U |
| 5570 | chr22 | 32021249 | 32021583 | Neuron CNS | 0.16 | 0.87 | intron | PISD | U |
| 5571 | chr22 | 32020002 | 32020487 | Neuron CNS | 0.16 | 0.86 | intron | PISD | U |
| 5572 | chr19 | 51046227 | 51046366 | Neuron CNS | 0.15 | 0.83 | intron | LRRC4B | U |
| 5573 | chr10 | 11343686 | 11343857 | Neuron CNS | 0.27 | 0.95 | intron | CELF2 | U |
| 5574 | chr11 | 1442511 | 1442732 | Neuron CNS | 0.21 | 0.88 | intron | BRSK2 | U |
| 5575 | chr20 | 4875680 | 4875959 | Neuron CNS | 0.17 | 0.84 | intron | SLC23A2 | U |
| 5576 | chr3 | 10541056 | 10541270 | Neuron CNS | 0.2 | 0.86 | intron | ATP282 | U |
| 5577 | chr22 | 50764436 | 50764729 | Neuron CNS | 0.16 | 0.82 | intron | DENND6B | U |
| 5578 | chr17 | 30821925 | 30822252 | Neuron CNS | 0.25 | 0.91 | exon, intron | MYO1D, MYO1D | U |
| 5579 | chr12 | 122839918 | 122840368 | Neuron CNS | 0.26 | 0.92 | intron | CLIP1 | U |
| 5580 | chr7 | 158045060 | 158045188 | Neuron CNS | 0.19 | 0.84 | intron | PTPRN2 | U |
| 5581 | chr8 | 417522643 | 41752432 | Neuron CNS | 0.18 | 0.83 | intron | ANK1 | U |
| 5582 | chr19 | 19391733 | 19391933 | Neuron CNS | 0.25 | 0.9 | intron | SUGP1 | U |
| 5583 | chr4 | 3531816 | 3531988 | Neuron CNS | 0.19 | 0.83 | intron | LRPAP1 | U |
| 5584 | chr12 | 1948026 | 1948146 | Neuron CNS | 0.23 | 0.86 | intron | CACNA2D4 | U |
| 5585 | chr16 | 89531912 | 89532036 | Neuron CNS | 0.24 | 0.87 | intron | ANKRD11 | U |
| 5586 | chr10 | 126694723 | 126694905 | Neuron CNS | 0.32 | 0.92 | intron | CTBP2 | U |
| 5587 | chr4 | 745666 | 745966 | Neuron CNS | 0.25 | 0.85 | intron | PCGF3 | U |
| 5588 | chr17 | 8380266 | 8380434 | Neuron CNS | 0.34 | 0.93 | intron, exon | MYH10, MYH10 | U |
| 5589 | chr10 | 1266945171 | 126694669 | Neuron CNS | 0.37 | 0.94 | intron | CTBP2 | U |
| 5590 | chr1 | 3695942 | 3696334 | Neuron CNS | 0.33 | 0.9 | TTS | LRRC47 | U |
| 5591 | chr3 | 133610529 | 133610997 | Neuron CNS | 0.35 | 0.91 | intron | RAB6B | U |
| 5592 | chr19 | 591318 | 591352 | Neuron CNS | 0.23 | 0.77 | intron | HCN2 | U |
| 5593 | chr9 | 35831106 | 35831443 | Neuron CNS | 0.28 | 0.82 | intron | TMEM8B | U |
| 5594 | chr16 | 255823 | 256318 | Neuron CNS | 0.36 | 0.9 | exon | LUC7L | U |
| 5595 | chr4 | 48130075 | 48130182 | Neuron CNS | 0.83 | 0.08 | intron | TXK | M |
| 5596 | chr3 | 49941216 | 49941255 | Neuron CNS | 0.87 | 0.14 | promoter-TSS | MST1R | M |
| 5597 | chr2 | 58274587 | 58274650 | Neuron CNS | 0.77 | 0.06 | intron | VRK2 | M |
| 5598 | chr17 | 78193304 | 78193679 | Neuron CNS | 0.77 | 0.06 | promoter-TSS | SLC26A11 | M |
| 5599 | chr16 | 26963425 | 26963518 | Neuron CNS | 0.79 | 0.1 | Intergenic | C16orf82 | M |
| 5600 | chr16 | 4250130 | 4250420 | Neuron CNS | 0.75 | 0.07 | intron | SRL | M |
| 5601 | chr19 | 10541834 | 10542021 | Neuron CNS | 0.77 | 0.1 | intron | PDE4A | M |
| 5602 | chr1 | 5720367 | 5720614 | Neuron CNS | 0.79 | 0.13 | Intergenic | MIR4417 | M |
| 5603 | chr20 | 61805264 | 61805657 | Neuron CNS | 0.78 | 0.12 | Intergenic | MIR124-3 | M |
| 5604 | chr8 | 112410169 | 112410632 | Neuron CNS | 0.86 | 0.22 | Intergenic | MIR2053 | M |
| 5605 | chr3 | 129047663 | 129047724 | Neuron CNS | 0.83 | 0.21 | Intergenic | H1FX | M |
| 5606 | chr4 | 3786115 | 3786290 | Neuron CNS | 0.81 | 0.19 | Intergenic | ADRA2C | M |
| 5607 | chr5 | 141132842 | 141133060 | Neuron CNS | 0.76 | 0.16 | Intergenic | ARAP3 | M |
| 5608 | chr10 | 16479398 | 16479508 | Neuron CNS | 0.75 | 0.17 | exon | PTER | M |
| 5609 | chr17 | 75911013 | 75911051 | Neuron CNS | 0.68 | 0.11 | Intergenic | FLI45079 | M |
| 5610 | chr7 | 1373114061 | 137311605 | Neuron CNS | 0.66 | 0.09 | intron | DGKI | M |
| 5611 | chr15 | 45722312 | 45722352 | Neuron CNS | 0.77 | 0.21 | promoter-TSS | C15orf48 | M |
| 5612 | chr13 | 304982441 | 30498499 | Neuron CNS | 0.62 | 0.07 | Intergenic | LINC00544 | M |
| 5613 | chr5 | 122180341 | 122180456 | Neuron CNS | 0.59 | 0.05 | promoter-TSS | SNX24 | M |
| 5614 | chr14 | 24457825 | 24458119 | Neuron CNS | 0.84 | 0.03 | promoter-TSS | DHRS4L2 | M |
| 5615 | chr15 | 89962708 | 89962940 | Neuron CNS | 0.8 | 0.06 | Intergenic | LOC254559 | M |
| 5616 | chr7 | 140103734 | 140104223 | Neuron CNS | 0.73 | 0.05 | promoter-TSS | RAB19 | M |
| 5617 | chr6 | 26088238 | 26088422 | Neuron CNS | 0.72 | 0.12 | intron | HFE | M |
| 5618 | chr22 | 35626962 | 35627117 | Neuron CNS | 0.81 | 0.28 | Intergenic | HMGX84 | M |
| 5619 | chr3 | 499412631 | 49941579 | Neuron CNS | 0.57 | 0.05 | promoter-TSS | MST1R | M |
| 5620 | chr10 | 102692483 | 102692493 | Oligodendrocytes | 0.08 | 0.96 | intron | FAM178A | U |
| 5621 | chr18 | 8639631 | 8639795 | Oligodendrocytes | 0.09 | 0.97 | TTS | RAB12 | U |
| 5622 | chr4 | 2877658 | 2877778 | Oligodendrocytes | 0.07 | 0.95 | exon | ADD1 | U |
| 5623 | chr17 | 4012880 | 4013162 | Oligodendrocytes | 0.07 | 0.95 | intron | ZZEF1 | U |
| 5624 | chr16 | 50830354 | 50830509 | Oligodendrocytes | 0.07 | 0.93 | exon | CYLD | U |
| 5625 | chr4 | 185707763 | 185707950 | Oligodendrocytes | 0.08 | 0.94 | intron | ACSL1 | U |
| 5626 | chr9 | 129167537 | 129167628 | Oligodendrocytes | 0.08 | 0.93 | intron | MVB12B | U |
| 5627 | chr13 | 41148403 | 41148627 | Oligodendrocytes | 0.05 | 0.89 | intron | FOXO1 | U |
| 5628 | chr16 | 72460443 | 2460715 | Oligodendrocytes | 0.1 | 0.94 | Intergenic | PMFBP1 | U |
| 5629 | chr18 | 53014425 | 53014712 | Oligodendrocytes | 0.05 | 0.89 | intron | TCF4 | U |
| 5630 | chr6 | 109153205 | 109153450 | Oligodendrocytes | 0.08 | 0.91 | Intergenic | ARMC2 | U |
| 5631 | chr12 | 125474255 | 125474586 | Oligodendrocytes | 0.11 | 0.93 | promoter-TSS | DHX37 | U |
| 5632 | chr18 | 77113101 | 77113386 | Oligodendrocytes | 0.12 | 0.94 | intron | ATP98 | U |
| 5633 | chr7 | 638594 | 639011 | Oligodendrocytes | 0.13 | 0.95 | intron | PRKAR1B | U |
| 5634 | chr15 | 45068518 | 45068593 | Oligodendrocytes | 0.11 | 0.92 | Intergenic | TRIM69 | U |
| 5635 | chr1 | 2006859 | 2007310 | Oligodendrocytes | 0.1 | 0.91 | intron | PRKCZ | U |
| 5636 | chr17 | 11973045 | 11973493 | Oligodendrocytes | 0.13 | 0.93 | intron | MAP2K4 | U |
| 5637 | chr1 | 243633926 | 243634391 | Oligodendrocytes | 0.12 | 0.92 | intron | SDCCAG8 | U |
| 5638 | chr18 | 74710903 | 74711313 | Oligodendrocytes | 0.14 | 0.93 | intron | MBP | U |
| 5639 | chr6 | 137027578 | 137027797 | Oligodendrocytes | 0.12 | 0.9 | intron | MAP3KS | U |
| 5640 | chr8 | 140889295 | 140889564 | Oligodendrocytes | 0.15 | 0.91 | intron | TRAPPC9 | U |
| 5641 | chr18 | 74713115 | 74713577 | Oligodendrocytes | 0.17 | 0.93 | intron | MBP | U |
| 5642 | chr8 | 26445968 | 26446351 | Oligodendrocytes | 0.17 | 0.91 | intron | DPYSL2 | U |
| 5643 | chr1 | 155842984 | 155843210 | Oligodendrocytes | 0.19 | 0.9 | intron | SYT11 | U |
| 5644 | chr15 | 43811799 | 43812296 | Oligodendrocytes | 0.18 | 0.89 | intron | MAP1A | U |
| 5645 | chr13 | 25743968 | 25744147 | Oligodendrocytes | 0.2 | 0.9 | exon | AMER2 | U |
| 5646 | chr8 | 26454586 | 26455026 | Oligodendrocytes | 0.26 | 0.93 | intron | DPYSL2 | U |
| 5647 | chr6 | 161677637 | 161677997 | Oligodendrocytes | 0.28 | 0.93 | intron | AGPAT4 | U |
| 5648 | chr4 | 153153323 | 153153793 | Oligodendrocytes | 0.22 | 0.86 | Intergenic | MIR3140 | U |
| 5649 | chr8 | 26438745 | 26439132 | Oligodendrocytes | 0.28 | 0.91 | intron | DPYSL2 | U |
| 5650 | chr5 | 173293925 | 173294107 | Oligodendrocytes | 0.06 | 0.95 | Intergenic | CPEB4 | U |
| 5651 | chr9 | 126391558 | 126391765 | Oligodendrocytes | 0.05 | 0.93 | intron | DENND1A | U |
| 5652 | chr3 | 93611888 | 93611944 | Oligodendrocytes | 0.08 | 0.95 | intron | PROS1 | U |
| 5653 | chr15 | 23066819 | 23066894 | Oligodendrocytes | 0.07 | 0.94 | intron | NIPA1 | U |
| 5654 | chr21 | 38223581 | 38223730 | Oligodendrocytes | 0.03 | 0.9 | intron | HLCS | U |
| 5655 | chr4 | 154011119 | 154011294 | Oligodendrocytes | 0.05 | 0.92 | Intergenic | TRIM2 | U |
| 5656 | chr3 | 1390903701 | 139090576 | Oligodendrocytes | 0.06 | 0.93 | intron | COPB2 | U |
| 5657 | chr | 731582 | 731790 | Oligodendrocytes | 0.05 | 0.92 | intron | KANK1 | U |
| 5658 | chr1 | 25580876 | 25581030 | Oligodendrocytes | 0.07 | 0.93 | Intergenic | C1orf63 | U |
| 5659 | chr7 | 660458613 | 66046015 | Oligodendrocytes | 0.08 | 0.94 | Intergenic | LOC493754 | U |
| 5660 | chr1 | 32793778 | 32794083 | Oligodendrocytes | 0.06 | 0.92 | intron | HDAC1 | U |
| 5661 | chr11 | 6421632 | 6422026 | Oligodendrocytes | 0.09 | 0.95 | TTS | APBB1 | U |
| 5662 | chr10 | 115069451 | 115069850 | Oligodendrocytes | 0.06 | 0.92 | Intergenic | HABP2 | U |
| 5663 | chr10 | 31070570 | 31070750 | Oligodendrocytes | 0.02 | 0.87 | Intergenic | LYZL2 | U |
| 5664 | chr9 | 117110058 | 117110260 | Oligodendrocytes | 0.05 | 0.9 | exon | AKNA | U |
| 5665 | chr3 | 85626381 | 85626587 | Oligodendrocytes | 0.06 | 0.91 | intron | CADM2 | U |
| 5666 | chr7 | 39500433 | 3950279 | Oligodendrocytes | 0.07 | 0.92 | intron | SDK1 | U |
| 5667 | chr18 | 44614183 | 44614525 | Oligodendrocytes | 0.05 | 0.9 | intron | KATNAL2 | U |
| 5668 | chr8 | 29039468 | 29039860 | Oligodendrocytes | 0.09 | 0.94 | intron | KIF138 | U |
| 5669 | chr2 | 111407869 | 111408368 | Oligodendrocytes | 0.1 | 0.95 | exon | BUB1 | U |
| 5670 | chr8 | 99034900 | 99035054 | Oligodendrocytes | 0.07 | 0.91 | intron | MATN2 | U |
| 5671 | chr5 | 112683322 | 112683509 | Oligodendrocytes | 0.07 | 0.91 | Intergenic | RFPL4B | U |
| 5672 | chr1 | 25981415 | 25981669 | Oligodendrocytes | 0.07 | 0.91 | intron | MANIC1 | U |
| 5673 | chr21 | 34050957 | 34051234 | Oligodendrocytes | 0.1 | 0.94 | intron | SYNJ1 | U |
| 5674 | chr19 | 12771697 | 12772078 | Oligodendrocytes | 0.1 | 0.94 | intron, exon | MAN2B1, MAN2B1 | U |
| 5675 | chr8 | 1408465493 | 140847025 | Oligodendrocytes | 0.08 | 0.92 | intron | TRAPPC9 | U |
| 5676 | chr11 | 34200124 | 34200210 | Oligodendrocytes | 0.1 | 0.93 | intron | ABTB2 | U |
| 5677 | chr7 | 47852597 | 47852706 | Oligodendrocytes | 0.09 | 0.92 | intron | PKD1L1 | U |
| 5678 | chr7 | 45722501 | 45722746 | Oligodendrocytes | 0.05 | 0.88 | intron | ADCY1 | U |
| 5679 | chr2 | 241279723 | 241279990 | Oligodendrocytes | 0.06 | 0.89 | Intergenic | GPC1 | U |
| 5680 | chr16 | 19884310 | 19884614 | Oligodendrocytes | 0.04 | 0.87 | intron | GPRC5B | U |
| 5681 | chr5 | 156882998 | 156883325 | Oligodendrocytes | 0.08 | 0.91 | Intergenic | NIPAL4 | U |
| 5682 | chr13 | 25766851 | 25767213 | Oligodendrocytes | 0.04 | 0.87 | Intergenic | AMER2 | U |
| 5683 | chr6 | 110996079 | 110996470 | Oligodendrocytes | 0.12 | 0.95 | intron | CDK19 | U |
| 5684 | chr18 | 74703472 | 74703970 | Oligodendrocytes | 0.1 | 0.93 | intron | MBP | U |
| 5685 | chr4 | 124219474 | 124219592 | Oligodendrocytes | 0.12 | 0.94 | intron | SPATA5 | U |
| 5686 | chr10 | 71002953 | 71003078 | Oligodendrocytes | 0.04 | 0.86 | exon | HKDC1 | U |
| 5687 | chr7 | 61652 | 61846 | Oligodendrocytes | 0.06 | 0.88 | Intergenic | FAM20C | U |
| 5688 | chr12 | 111930410 | 111930725 | Oligodendrocytes | 0.12 | 0.94 | intron | ATXN2 | U |
| 5689 | chr3 | 54952905 | 54953270 | Oligodendrocytes | 0.08 | 0.9 | exon | LRTM1 | U |
| 5690 | chr18 | 72201352 | 72201786 | Oligodendrocytes | 0.08 | 0.9 | promoter-TSS | CNDP1 | U |
| 5691 | chr18 | 5969627 | 5969695 | Oligodendrocytes | 0.11 | 0.92 | exon, intron | L3MBTL4, L3MBTL4 | U |
| 5692 | chr10 | 1145347101 | 114535051 | Oligodendrocytes | 0.11 | 0.92 | intron | VTI1A | U |
| 5693 | chr18 | 74721088 | 74721495 | Oligodendrocytes | 0.07 | 0.88 | intron | MBP | U |
| 5694 | chr13 | 67730620 | 67731037 | Oligodendrocytes | 0.07 | 0.88 | intron | PCDH9 | U |
| 5695 | chr14 | 103504564 | 103504989 | Oligodendrocytes | 0.14 | 0.95 | intron | CDC42BPB | U |
| 5696 | chr1 | 51417651 | 51418121 | Oligodendrocytes | 0.15 | 0.96 | intron | FAF1 | U |
| 5697 | chr8 | 41474391 | 41474862 | Oligodendrocytes | 0.12 | 0.93 | intron | AGPAT6 | U |
| 5698 | chr18 | 13635908 | 13636384 | Oligodendrocytes | 0.08 | 0.89 | intron | LDLRAD4 | U |
| 5699 | chr19 | 47179636 | 47179986 | Oligodendrocytes | 0.09 | 0.89 | intron | PRKD2 | U |
| 5700 | chr5 | 38678704 | 38679161 | Oligodendrocytes | 0.11 | 0.91 | Intergenic | LIFR | U |
| 5701 | chr2 | 71773925 | 71774038 | Oligodendrocytes | 0.13 | 0.92 | intron | DYSF | U |
| 5702 | chr6 | 151054734 | 151054902 | Oligodendrocytes | 0.15 | 0.94 | exon | PLEKHG1 | U |
| 5703 | chr13 | 27566274 | 27566503 | Oligodendrocytes | 0.12 | 0.91 | Intergenic | USP12 | U |
| 5704 | chr20 | 403897 | 404129 | Oligodendrocytes | 0.14 | 0.93 | intron | RBCK1 | U |
| 5705 | chr6 | 144335615 | 144335871 | Oligodendrocytes | 0.09 | 0.88 | intron | PLAGLI | U |
| 5706 | chr17 | 31192037 | 31192355 | Oligodendrocytes | 0.17 | 0.96 | intron | MYO1D | U |
| 5707 | chr18 | 74710513 | 74710710 | Oligodendrocytes | 0.11 | 0.89 | intron | MBP | U |
| 5708 | chr8 | 140804332 | 140804788 | Oligodendrocytes | 0.15 | 0.93 | intron | TRAPPC9 | U |
| 5709 | chr13 | 113404881 | 113405378 | Oligodendrocytes | 0.12 | 0.9 | intron | ATP11A | U |
| 5710 | chr3 | 183517689 | 183517916 | Oligodendrocytes | 0.17 | 0.94 | intron | YEATS2 | U |
| 5711 | chr1 | 36658407 | 36658705 | Oligodendrocytes | 0.15 | 0.92 | Intergenic | THRAP3 | U |
| 5712 | chr21 | 45644734 | 45645108 | Oligodendrocytes | 0.14 | 0.91 | Intergenic | ICOSLG | U |
| 5713 | chr19 | 55799672 | 55799913 | Oligodendrocytes | 0.12 | 0.88 | intron | BRSK1 | U |
| 5714 | chr2 | 55186307 | 55186637 | Oligodendrocytes | 0.18 | 0.94 | intron | EML6 | U |
| 5715 | chr16 | 75332090 | 75332557 | Oligodendrocytes | 0.11 | 0.87 | intron | CFDP1 | U |
| 5716 | chr1 | 54584079 | 54584345 | Oligodendrocytes | 0.13 | 0.85 | Intergenic | CDCP2 | U |
| 5717 | chr20 | 52711064 | 52711192 | Oligodendrocytes | 0.18 | 0.92 | Intergenic | BCAS1 | U |
| 5718 | chr18 | 10442102 | 10442261 | Oligodendrocytes | 0.2 | 0.94 | Intergenic | APCDD1 | U |
| 5719 | chr12 | 121282907 | 121283256 | Oligodendrocytes | 0.21 | 0.94 | intron | SPPL3 | U |
| 5720 | chr18 | 77137458 | 77137866 | Oligodendrocytes | 0.17 | 0.9 | exon | ATP9B | U |
| 5721 | chr21 | 45650440 | 45650910 | Oligodendrocytes | 0.18 | 0.9 | intron | ICOSLG | U |
| 5722 | chr16 | 74782126 | 74782286 | Oligodendrocytes | 0.05 | 0.88 | intron | FA2H | U |
| 5723 | chr4 | 55127142 | 55127488 | Oligodendrocytes | 0.06 | 0.88 | intron | PDGFRA | U |
| 5724 | chr3 | 194109864 | 194110213 | Oligodendrocytes | 0.13 | 0.91 | Intergenic | GP5 | U |
| 5725 | chr20 | 37138022 | 37138098 | Oligodendrocytes | 0.05 | 0.96 | intron | RALGAPB | U |
| 5726 | chr12 | 123332891 | 12333035 | Oligodendrocytes | 0.03 | 0.91 | intron | HIP1R | U |
| 5727 | chr9 | 115509635 | 115509918 | Oligodendrocytes | 0.05 | 0.93 | Intergenic | SNX30 | U |
| 5728 | chr6 | 17839739 | 17840026 | Oligodendrocytes | 0.06 | 0.93 | intron | KIF13A | U |
| 5729 | chr14 | 52657938 | 52658001 | Oligodendrocytes | 0.05 | 0.92 | Intergenic | PTGDR | U |
| 5730 | chr18 | 74721776 | 74721930 | Oligodendrocytes | 0.08 | 0.94 | intron | MBP | U |
| 5731 | chr3 | 42190918 | 42191192 | Oligodendrocytes | 0.03 | 0.88 | exon | TRAK1 | U |
| 5732 | chr12 | 120951809 | 120952170 | Oligodendrocytes | 0.05 | 0.9 | intron | COQ5 | U |
| 5733 | chr14 | 34257319 | 34257708 | Oligodendrocytes | 0.07 | 0.91 | intron | NPAS3 | U |
| 5734 | chr11 | 604874 | 605237 | Oligodendrocytes | 0.12 | 0.94 | exon, intron | PHRF1 | U |
| 5735 | chr17 | 30821748 | 30821863 | Oligodendrocytes | 0.09 | 0.89 | exon | MYO1D | U |
| 5736 | chr8 | 77316589 | 77316860 | Oligodendrocytes | 0.07 | 0.87 | Intergenic | ZFHX4 | U |
| 5737 | chr8 | 25542884 | 25542961 | Oligodendrocytes | 0.11 | 0.9 | Intergenic | CDCA2 | U |
| 5738 | chr19 | 36267294 | 36267628 | Oligodendrocytes | 0.11 | 0.9 | intron | ARHGAP33 | U |
| 5739 | chr2 | 160087061 | 160087497 | Oligodendrocytes | 0.15 | 0.94 | exon | TANC1 | U |
| 5740 | chr10 | 131694675 | 131694772 | Oligodendrocytes | 0.13 | 0.89 | intron | EBF3 | U |
| 5741 | chr7 | 158044788 | 158045029 | Oligodendrocytes | 0.09 | 0.85 | intron | PTPRN2 | U |
| 5742 | chr8 | 25543136 | 25543236 | Oligodendrocytes | 0.13 | 0.88 | Intergenic | COCA2 | U |
| 5743 | chr22 | 31091742 | 31092169 | Oligodendrocytes | 0.21 | 0.91 | intron | OSBP2 | U |
| 5744 | chr9 | 73231641 | 73231771 | Oligodendrocytes | 0.22 | 0.91 | intron | TRPM3 | U |
| 5745 | chr19 | 58447249 | 58447435 | Oligodendrocytes | 0.04 | 0.94 | promoter-TSS | ZNF418 | U |
| 5746 | chr17 | 76455195 | 76455319 | Oligodendrocytes | 0.03 | 0.92 | exon | DNAH17 | U |
| 5747 | chr7 | 27591588 | 27591670 | Oligodendrocytes | 0.05 | 0.93 | intron | HIBADH | U |
| 5748 | chr9 | 92051265 | 92051357 | Oligodendrocytes | 0.07 | 0.95 | intron | SEMA4D | U |
| 5749 | chr16 | 75129190 | 75129361 | Oligodendrocytes | 0.05 | 0.93 | intron | ZNRF1 | U |
| 5750 | chr18 | 74728663 | 74729022 | Oligodendrocytes | 0.04 | 0.92 | exon | MBP | U |
| 5751 | chr12 | 12397259 | 12397688 | Oligodendrocytes | 0.06 | 0.94 | intron | LRP6 | U |
| 5752 | chr9 | 134836851 | 134837236 | Oligodendrocytes | 0.08 | 0.95 | intron | MED27 | U |
| 5753 | chr18 | 74721791 | 74721931 | Oligodendrocytes | 0.08 | 0.94 | intron | MBP | U |
| 5754 | chr7 | 129565080 | 129565368 | Oligodendrocytes | 0.04 | 0.9 | intron | UBE2H | U |
| 5755 | chr6 | 3441986 | 3442479 | Oligodendrocytes | 0.08 | 0.94 | intron | SLC22A23 | U |
| 5756 | chr20 | 34807783 | 34808023 | Oligodendrocytes | 0.07 | 0.92 | intron | EPB41L1 | U |
| 5757 | chr16 | 69833813 | 69834127 | Oligodendrocytes | 0.07 | 0.92 | intron | WWP2 | U |
| 5758 | chr5 | 20096860 | 20097330 | Oligodendrocytes | 0.06 | 0.91 | Intergenic | MBOAT1 | U |
| 5759 | chr2 | 101181112 | 101181337 | Oligodendrocytes | 0.09 | 0.93 | intron | PDCL3 | U |
| 5760 | chr11 | 63818827 | 63819149 | Oligodendrocytes | 0.1 | 0.94 | intron | MACROD1 | U |
| 5761 | chr9 | 100769817 | 100769975 | Oligodendrocytes | 0.08 | 0.9 | intron | ANP328 | U |
| 5762 | chr12 | 110487715 | 110487908 | Oligodendrocytes | 0.1 | 0.92 | intron | C12orf76 | U |
| 5763 | chr22 | 38479240 | 38479487 | Oligodendrocytes | 0.09 | 0.91 | promoter-TSS | SLC16A8 | U |
| 5764 | chr19 | 7917329 | 7917550 | Oligodendrocytes | 0.08 | 0.89 | intron | EVI5L | U |
| 5765 | chr20 | 47354006 | 47354417 | Oligodendrocytes | 0.09 | 0.89 | intron | PREX1 | U |
| 5766 | chr20 | 36502306 | 36502753 | Oligodendrocytes | 0.16 | 0.93 | Intergenic | VSTM2L | U |
| 5767 | chr20 | 37008012 | 37008338 | Oligodendrocytes | 0.12 | 0.88 | Intergenic | LBP | U |
| 5768 | chr19 | 7916744 | 7917217 | Oligodendrocytes | 0.12 | 0.88 | intron | EVI5L | U |
| 5769 | chr6 | 15212742 | 15213225 | Oligodendrocytes | 0.2 | 0.93 | Intergenic | JARID2 | U |
| 5770 | chr2 | 3498878 | 3499194 | Oligodendrocytes | 0.19 | 0.91 | Intergenic | ADI1 | U |
| 5771 | chr10 | 134549977 | 134550161 | Oligodendrocytes | 0.23 | 0.92 | intron | INPP5A | U |
| 5772 | chr8 | 1765444 | 1765558 | Oligodendrocytes | 0.87 | 0.12 | TTS | MIR596 | M |
| 5773 | chr8 | 1765080 | 1765430 | Oligodendrocytes | 0.81 | 0.06 | promoter-TSS | MIR596 | M |
| 5774 | chr11 | 126455766 | 126456053 | Oligodendrocytes | 0.75 | 0.03 | intron | KIRREL3 | M |
| 5775 | chr1 | 37945273 | 37945559 | Oligodendrocytes | 0.76 | 0.05 | intron | ZC3H12A | M |
| 5776 | chr4 | 41219007 | 41219224 | Oligodendrocytes | 0.77 | 0.09 | Intergenic | APBB2 | M |
| 5777 | chrX | 25040201 | 25040398 | Oligodendrocytes | 0.88 | 0.23 | Intergenic | ARX | M |
| 5778 | chr3 | 50638378 | 50638841 | Oligodendrocytes | 0.67 | 0.02 | Intergenic | CISH | M |
| 5779 | chr6 | 43423853 | 43424084 | Oligodendrocytes | 0.76 | 0.15 | promoter-TSS | DLK2 | M |
| 5780 | chr9 | 135039351 | 135039635 | Oligodendrocytes | 0.65 | 0.06 | intron | NTNG2 | M |
| 5781 | chr10 | 88471584 | 88472060 | Oligodendrocytes | 0.59 | 0.01 | intron | LDB3 | M |
| 5782 | chr6 | 35997057 | 35997540 | Oligodendrocytes | 0.57 | 0.03 | intron | MAPK14 | M |
| 5783 | chr11 | 31831226 | 31831349 | Oligodendrocytes | 0.91 | 0.17 | intron | PAX6 | M |
| 5784 | chr11 | 31829278 | 31829432 | Oligodendrocytes | 0.93 | 0.14 | intron | PAX6 | M |
| 5785 | chr8 | 65291068 | 65291430 | Oligodendrocytes | 0.88 | 0.1 | promoter-TSS | MIR124-2 | M |
| 5786 | chr11 | 31825751 | 31825834 | Oligodendrocytes | 0.88 | 0.16 | intron | PAX6 | M |
| 5787 | chr4 | 8868932 | 8869286 | Oligodendrocytes | 0.8 | 0.08 | TTS, exon | HMX1, HMX1 | M |
| 5788 | chr11 | 31824170 | 31824375 | Oligodendrocytes | 0.83 | 0.13 | intron | PAX6 | M |
| 5789 | chr11 | 31826068 | 31826108 | Oligodendrocytes | 0.87 | 0.18 | intron | PAX6 | M |
| 5790 | chr8 | 65292442 | 65292662 | Oligodendrocytes | 0.74 | 0.06 | TTS | MIR124-2 | M |
| 5791 | chr8 | 55292680 | 65292939 | Oligodendrocytes | 0.81 | 0.13 | TTS, Intergenic | MIR124-2, MIR124-2 | M |
| 5792 | chr20 | 21498239 | 21498692 | Oligodendrocytes | 0.77 | 0.11 | Intergenic | NKX2-2 | M |
| 5793 | chr20 | 21497889 | 21498176 | Oligodendrocytes | 0.72 | 0.07 | Intergenic | NKX2-2 | M |
| 5794 | chr11 | 31824580 | 31825034 | Oligodendrocytes | 0.76 | 0.11 | intron | PAX6 | M |
| 5795 | chr4 | 8870018 | 3870241 | Oligodendrocytes | 0.73 | 0.1 | intron | HMX1 | M |
| 5796 | chr20 | 21500838 | 21501256 | Oligodendrocytes | 0.56 | 0.1 | Intergenic | NKX2-2 | M |
| 5797 | chr18 | 52721860 | 52722004 | Neurons + Oligodendrocytes | 0.08 | 0.89 | Intergenic | CCDC68 | U |
| 5798 | chr | 70367594 | 70367840 | Neurons + Oligodendrocytes | 0.08 | 0.88 | exon | NLGN3 | U |
| 5799 | chr7 | 158159035 | 158159135 | Neurons + Oligodendrocytes | 0.11 | 0.88 | intron | PTPRN2 | U |
| 5800 | chr8 | 9260354 | 9260504 | Neurons + Oligodendrocytes | 0.06 | 0.83 | Intergenic | LOC157273 | U |
| 5801 | chr | 2006365 | 2006616 | Neurons + Oligodendrocytes | 0.06 | 0.83 | intron | PRKCZ | U |
| 5802 | chr4 | 184829257 | 184829322 | Neurons + Oligodendrocytes | 0.11 | 0.87 | intron | STOX2 | U |
| 5803 | chr17 | 27909733 | 27910182 | Neurons + Oligodendrocytes | 0.13 | 0.89 | intron, exon | GIT1, GIT1 | U |
| 5804 | chr19 | 51052053 | 51052114 | Neurons + Oligodendrocytes | 0.06 | 0.81 | exon | LRRC4B | U |
| 5805 | chr9 | 27603240 | 27603332 | Neurons + Oligodendrocytes | 0.18 | 0.93 | intron | CCDC25 | U |
| 5806 | chr9 | 124846277 | 124846678 | Neurons + Oligodendrocytes | 0.12 | 0.87 | intron | TTLL11 | U |
| 5807 | chr12 | 20025836 | 20025925 | Neurons + Oligodendrocytes | 0.19 | 0.93 | Intergenic | LOC100506393 | U |
| 5808 | chr13 | 113698606 | 113698822 | Neurons + Oligodendrocytes | 0.07 | 0.81 | intron | MCF2L | U |
| 5809 | chr19 | 4357207 | 4357406 | Neurons + Oligodendrocytes | 0.17 | 0.9 | exon | MPND | U |
| 5810 | chr1 | 160062107 | 160062378 | Neurons + Oligodendrocytes | 0.2 | 0.93 | intron | IGSF8 | U |
| 5811 | chr10 | 77211693 | 77212130 | Neurons + Oligodendrocytes | 0.11 | 0.84 | Intergenic | ZNF503-AS2 | U |
| 5812 | chr2 | 74612706 | 74613160 | Neurons + Oligodendrocytes | 0.12 | 0.85 | promoter-TSS | DCTN1-AS1 | U |
| 5813 | chr15 | 22996654 | 22996878 | Neurons + Oligodendrocytes | 0.19 | 0.91 | intron | CYFIP1 | U |
| 5814 | chr8 | 26446533 | 26446811 | Neurons + Oligodendrocytes | 0.15 | 0.87 | intron | DPYSL2 | U |
| 5815 | chr12 | 49578469 | 49578945 | Neurons + Oligodendrocytes | 0.08 | 0.8 | exon | TUBA1A | U |
| 5816 | chr5 | 179711246 | 179711444 | Neurons + Oligodendrocytes | 0.16 | 0.87 | intron | MAPK9 | U |
| 5817 | chr11 | 63831218 | 63831535 | Neurons + Oligodendrocytes | 0.2 | 0.91 | intron | MACROD1 | U |
| 5818 | chr19 | 2633340 | 2633399 | Neurons + Oligodendrocytes | 0.15 | 0.85 | intron | GNG7 | U |
| 5819 | chr11 | 63451515 | 63451644 | Neurons + Oligodendrocytes | 0.2 | 0.9 | intron | RTN3 | U |
| 5820 | chr4 | 29793183 | 2979501 | Neurons + Oligodendrocytes | 0.15 | 0.85 | intron | GRK4 | U |
| 5821 | chrX | 70368045 | 70368367 | Neurons + Oligodendrocytes | 0.1 | 0.8 | intron | NLGN3 | U |
| 5822 | chr22 | 45605441 | 45605504 | Neurons + Oligodendrocytes | 0.15 | 0.84 | intron | KIAA0930 | U |
| 5823 | chr18 | 55532983 | 56533120 | Neurons + Oligodendrocytes | 0.14 | 0.83 | intron | ZNF532 | U |
| 5824 | chr11 | 45923416 | 45923589 | Neurons + Oligodendrocytes | 0.16 | 0.85 | intron | MAPK8IP1 | U |
| 5825 | chr2 | 74232167 | 74232425 | Neurons + Oligodendrocytes | 0.18 | 0.87 | Intergenic | TET3 | U |
| 5826 | chr16 | 1762773 | 1763090 | Neurons + Oligodendrocytes | 0.12 | 0.81 | intron | MAPK8IP3 | U |
| 5827 | chr11 | 20782466 | 20782532 | Neurons + Oligodendrocytes | 0.21 | 0.89 | intron | NELL1 | U |
| 5828 | chr13 | 25744404 | 25744495 | Neurons + Oligodendrocyte: | 0.08 | 0.76 | exon | AMER2 | U |
| 5829 | chr14 | 97129256 | 97129453 | Neurons + Oligodendrocytes | 0.2 | 0.88 | Intergenic | VRK1 | U |
| 5830 | chr16 | 73363089 | 73363306 | Neurons + Oligodendrocytes | 0.14 | 0.82 | Intergenic | LOC100506172 | U |
| 5831 | chr4 | 176732188 | 176732527 | Neurons + Oligodendrocytes | 0.19 | 0.87 | intron | GPM6A | U |
| 5832 | chr5 | 87951971 | 87952356 | Neurons + Oligodendrocytes | 0.1 | 0.78 | intron | LINC00461 | U |
| 5833 | chr17 | 64949606 | 64949705 | Neurons + Oligodendrocytes | 0.21 | 0.88 | Intergenic | CACNG4 | U |
| 5834 | chr18 | 34887267 | 34887534 | Neurons + Oligodendrocytes | 0.2 | 0.87 | intron | CELF4 | U |
| 5835 | chr11 | 6435640 | 6435942 | Neurons + Oligodendrocytes | 0.2 | 0.87 | intron | APBB1 | U |
| 5836 | chr4 | 176728591 | 176728931 | Neurons + Oligodendrocytes | 0.15 | 0.82 | intron | GPM6A | U |
| 5837 | chr11 | 19887608 | 19888049 | Neurons + Oligodendrocytes | 0.18 | 0.85 | intron | NAV2 | U |
| 5838 | chr7 | 138603863 | 138604108 | Neurons + Oligodendrocytes | 0.26 | 0.92 | exon | KIAA1549 | U |
| 5839 | chr6 | 157493920 | 157494198 | Neurons + Oligodendrocytes | 0.24 | 0.9 | intron | ARID1B | U |
| 5840 | chr19 | 26334521 | 2633481 | Neurons + Oligodendrocytes | 0.22 | 0.87 | intron | GNG7 | U |
| 5841 | chr4 | 58050932 | 58051071 | Neurons + Oligodendrocytes | 0.24 | 0.89 | intron | LOC255130 | U |
| 5842 | chr10 | 68686999 | 68687216 | Neurons + Oligodendrocytes | 0.16 | 0.81 | exon | LRRTM3 | U |
| 5843 | chr6 | 161687426 | 161687894 | Neurons + Oligodendrocytes | 0.27 | 0.92 | intron | AGPAT4 | U |
| 5844 | chr19 | 11090675 | 11090775 | Neurons + Oligodendrocytes | 0.23 | 0.83 | intron | SMARCA4 | U |
| 5845 | chr5 | 61028699 | 61028855 | Neurons + Oligodendrocytes | 0.19 | 0.82 | Intergenic | C5orf64 | U |
| 5846 | chr2 | 120473894 | 120474110 | Neurons + Oligodendrocytes | 0.21 | 0.84 | Intergenic | TMEM177 | U |
| 5847 | chr1 | 116929944 | 116930168 | Neurons + Oligodendrocytes | 0.26 | 0.89 | exon | ATP1A1 | U |
| 5848 | chr16 | 19276956 | 19277219 | Neurons + Oligodendrocytes | 0.21 | 0.84 | intron | SYT17 | U |
| 5849 | chr2 | 32702591 | 3270658 | Neurons + Oligodendrocytes | 0.24 | 0.87 | intron | TSSC1 | U |
| 5850 | chr19 | 33028433 | 33028585 | Neurons + Oligodendrocytes | 0.24 | 0.86 | Intergenic | PDCD5 | U |
| 5851 | chr2 | 241736359 | 241736733 | Neurons + Oligodendrocytes | 0.24 | 0.86 | intron | KIF1A | U |
| 5852 | chr8 | 63602731 | 63602856 | Neurons + Oligodendrocytes | 0.2 | 0.81 | intron | NKAIN3 | U |
| 5853 | chr15 | 292846501 | 29284812 | Neurons + Oligodendrocytes | 0.18 | 0.79 | intron | APBA2 | U |
| 5854 | chr16 | 11773368 | 11773555 | Neurons + Oligodendrocytes | 0.3 | 0.91 | TTS | SNN | U |
| 5855 | chr19 | 47999880 | 48000219 | Neurons + Oligodendrocytes | 0.25 | 0.86 | intron | NAPA | U |
| 5856 | chr1 | 26229961 | 26230278 | Neurons + Oligodendrocytes | 0.28 | 0.88 | intron, exon | STMN1, STMN1 | U |
| 5857 | chr4 | 185706178 | 185706627 | Neurons + Oligodendrocytes | 0.3 | 0.9 | intron | ACSL1 | U |
| 5858 | chr1 | 11543075 | 11543165 | Neurons + Oligodendrocytes | 0.22 | 0.81 | intron | PTCHD2 | U |
| 5859 | chr14 | 1050116503 | 105011772 | Neurons + Oligodendrocytes | 0.23 | 0.82 | Intergenic | C14orf180 | U |
| 5860 | chr8 | 30889770 | 30889896 | Neurons + Oligodendrocytes | 0.22 | 0.81 | promoter-TSS | WRN | U |
| 5861 | chr19 | 510516421 | 51051708 | Neurons + Oligodendrocytes | 0.23 | 0.81 | intron | LRRC4B | U |
| 5862 | chr11 | 1591878 | 1592102 | Neurons + Oligodendrocytes | 0.2 | 0.78 | intron | DUSP8 | U |
| 5863 | chr14 | 105783388 | 105783537 | Neurons + Oligodendrocytes | 0.31 | 0.88 | intron | PACS2 | U |
| 5864 | chr15 | 97118141 | 97118417 | Neurons + Oligodendrocytes | 0.29 | 0.85 | Intergenic | SPATA8 | U |
| 5865 | chr3 | 92734601 | 9273804 | Neurons + Oligodendrocytes | 0.32 | 0.88 | intron | SRGAP3 | U |
| 5866 | chr4 | 5860051 | 5860231 | Neurons + Oligodendrocytes | 0.28 | 0.83 | intron | CRMP1 | U |
| 5867 | chr3 | 119450493 | 119450905 | Neurons + Oligodendrocytes | 0.34 | 0.88 | intron | MAATS1 | U |
| 5868 | chr | 101517703 | 10151976 | Neurons + Oligodendrocytes | 0.22 | 0.75 | intron | CLCN4 | U |
| 5869 | chr8 | 48102533 | 48102819 | Neurons + Oligodendrocytes | 0.32 | 0.82 | exon | LOC100287846 | U |
| 5870 | chr5 | 141263876 | 141263978 | Neurons + Oligodendrocytes | 0.28 | 0.75 | Intergenic | PCDH1 | U |
| 5871 | chr10 | 3283867 | 3283912 | Neurons + Oligodendrocytes | 0.04 | 0.86 | Intergenic | PITRM1 | U |
| 5872 | chr13 | 101302863 | 101303067 | Neurons + Oligodendrocytes | 0.08 | 0.9 | intron | TMTC4 | U |
| 5873 | chr1 | 2005852 | 2006068 | Neurons + Oligodendrocytes | 0.04 | 0.86 | intron | PRKCZ | U |
| 5874 | chr9 | 96846792 | 96847041 | Neurons + Oligodendrocytes | 0.08 | 0.86 | exon | PTPDC1 | U |
| 5875 | chr13 | 113698901 | 113699209 | Neurons + Oligodendrocytes | 0.07 | 0.85 | intron | MCF2L | U |
| 5876 | chr18 | 530746281 | 53075071 | Neurons + Oligodendrocytes | 0.12 | 0.9 | intron | TCF4 | U |
| 5877 | chr20 | 24959344 | 24959581 | Neurons + Oligodendrocytes | 0.16 | 0.92 | exon | APMAP | U |
| 5878 | chr1 | 14113094 | 14113343 | Neurons + Oligodendrocytes | 0.18 | 0.94 | exon | PRDM2 | U |
| 5879 | chr16 | 88677591 | 88677848 | Neurons + Oligodendrocytes | 0.12 | 0.88 | exon, intron | ZC3H18, ZC3H18 | U |
| 5880 | chr8 | 139613769 | 139613819 | Neurons + Oligodendrocytes | 0.12 | 0.86 | intron | COL22A1 | U |
| 5881 | chr5 | 11564974 | 11565144 | Neurons + Oligodendrocytes | 0.14 | 0.88 | exon | CTNND2 | U |
| 5882 | chr10 | 131694516 | 131694773 | Neurons + Oligodendrocytes | 0.11 | 0.85 | intron | EBF3 | U |
| 5883 | chr15 | 82042494 | 82042715 | Neurons + Oligodendrocytes | 0.15 | 0.88 | Intergenic | MEX3B | U |
| 5884 | chr16 | 50897241 | 50897299 | Neurons + Oligodendrocytes | 0.24 | 0.94 | Intergenic | CYLD | U |
| 5885 | chr3 | 51731038 | 51731232 | Neurons + Oligodendrocytes | 0.22 | 0.91 | intron | TEX264 | U |
| 5886 | chr22 | 32020786 | 32021184 | Neurons + Oligodendrocytes | 0.2 | 0.88 | intron | PISD | U |
| 5887 | chr10 | 3283414 | 3283766 | Neurons + Oligodendrocytes | 0.24 | 0.91 | Intergenic | PITRM1 | U |
| 5888 | chr7 | 2815153 | 2815546 | Neurons + Oligodendrocytes | 0.24 | 0.91 | intron | GNA12 | U |
| 5889 | chr8 | 10001036 | 10001524 | Neurons + Oligodendrocytes | 0.22 | 0.89 | intron | MSRA | U |
| 5890 | chr12 | 50296224 | 50296444 | Neurons + Oligodendrocytes | 0.3 | 0.94 | intron | FAIM2 | U |
| 5891 | chr10 | 15000265 | 15000756 | Neurons + Oligodendrocytes | 0.26 | 0.9 | Intergenic, promote [symbol] ; | MEIG1, MEIG1 | U |
| 5892 | chr11 | 1420702 | 1420823 | Neurons + Oligodendrocytes | 0.15 | 0.78 | intron | BRSK2 | U |
| 5893 | chr19 | 51046227 | 51046366 | Neurons + Oligodendrocytes | 0.21 | 0.84 | intron | LRRC4B | U |
| 5894 | chr14 | 51559477 | 51559756 | Neurons + Oligodendrocytes | 0.23 | 0.86 | intron | TRIM9 | U |
| 5895 | chr14 | 69157866 | 69158048 | Neurons + Oligodendrocytes | 0.28 | 0.9 | Intergenic | ZFP36L1 | U |
| 5896 | chr16 | 88677852 | 88677939 | Neurons + Oligodendrocytes | 0.18 | 0.79 | exon | ZC3H18 | U |
| 897 | chr2 | 240320293 | 240320511 | Neurons + Oligodendrocytes | 0.26 | 0.84 | intron | HDAC4 | U |
| 5898 | chr22 | 27680331 | 27680730 | Neurons + Oligodendrocytes | 0.32 | 0.9 | Intergenic | MN1 | U |
| 5899 | chr1 | 48035596 | 48035785 | Neurons + Oligodendrocytes | 0.88 | 0.12 | Intergenic | FOXD2 | M |
| 5900 | chr12 | 124247229 | 124247359 | Neurons + Oligodendrocytes | 0.82 | 0.1 | TTS | ATP6V0A2 | M |
| 5901 | chr18 | 76529112 | 76529190 | Neurons + Oligodendrocytes | 0.86 | 0.15 | Intergenic | SALL3 | M |
| 5902 | chr19 | 46017956 | 46018119 | Neurons + Oligodendrocytes | 0.83 | 0.14 | intron | VASP | M |
| 5903 | chr14 | 94425764 | 94425928 | Neurons + Oligodendrocytes | 0.82 | 0.14 | intron | ASB2 | M |
| 5904 | chr1 | 37176500 | 37176889 | Neurons + Oligodendrocytes | 0.78 | 0.13 | Intergenic | CSF3R | M |
| 5905 | chr17 | 47563495 | 47563672 | Neurons + Oligodendrocytes | 0.86 | 0.24 | Intergenic | NGFR | M |
| 5906 | chr17 | 72353695 | 72353736 | Neurons + Oligodendrocytes | 0.78 | 0.19 | exon | BTBD17 | M |
| 5907 | chr19 | 13828284 | 13828420 | Neurons + Oligodendrocytes | 0.78 | 0.2 | Intergenic | CCDC130 | M |
| 5908 | chr4 | 3730359 | 3730506 | Neurons + Oligodendrocytes | 0.76 | 0.18 | Intergenic | ADRA2C | M |
| 5909 | chr3 | 53107216 | 53107541 | Neurons + Oligodendrocytes | 0.63 | 0.07 | Intergenic | SFMBT1 | M |
| 5910 | chr19 | 5430397 | 5430550 | Neurons + Oligodendrocytes | 0.75 | 0.2 | Intergenic | ZNRF4 | M |
| 5911 | chr19 | 46142858 | 46143018 | Neurons + Oligodendrocytes | 0.68 | 0.13 | promoter-TSS | EML2 | M |
| 5912 | chr8 | 41180988 | 41181399 | Neurons + Oligodendrocytes | 0.8 | 0.13 | Intergenic | SFRP1 | M |
| 5913 | chr14 | 70038634 | 70039091 | Neurons + Oligodendrocytes | 0.88 | 0.02 | exon | CCDC177 | M |
| 5914 | chr14 | 70039093 | 70039265 | Neurons + Oligodendrocytes | 0.86 | 0.04 | exon | CCDC177 | M |
| 5915 | chr12 | 49659174 | 49659537 | Neurons + Oligodendrocytes | 0.8 | 0.05 | intron | TUBA1C | M |
| 5916 | chr22 | 39437295 | 39437591 | Neurons + Oligodendrocytes | 0.88 | 0.16 | exon, intron | APOBEC3F, APOBEC3 [symbol] | M |
| 5917 | chr11 | 31840808 | 31840967 | Neurons + Oligodendrocytes | 0.89 | 0.18 | intron | DKFZp686K1684 | M |
| 5918 | chr14 | 24458196 | 24458283 | Neurons + Oligodendrocytes | 0.78 | 0.08 | exon | DHRS4L2 | M |
| 5919 | chr2 | 25354646 | 25354707 | Neurons + Oligodendrocytes | 0.84 | 0.17 | exon | EFR3B | M |
| 5920 | chr2 | 25354716 | 25355064 | Neurons + Oligodendrocytes | 0.76 | 0.09 | intron | EFR38 | M |
| 5921 | chr17 | 47563733 | 47563830 | Neurons + Oligodendrocytes | 0.76 | 0.11 | Intergenic | NGFR | M |
| 5922 | chr19 | 39898186 | 39898552 | Neurons + Oligodendrocytes | 0.67 | 0.02 | intron, exon | ZFP36, 2FP36 | M |
| 5923 | chr12 | 124246303 | 124246510 | Neurons + Oligodendrocytes | 0.63 | 0.06 | promoter-TSS | DNAH10 | M |
| 5924 | chr1 | 11174802 | 11175183 | Pancreatic Alpha + Beta + Delta cells | 0.01 | 0.93 | intron | MTOR | U |
| 5925 | chr18 | 39584405 | 39584730 | Pancreatic Alpha + Beta + Delta cells | 0.03 | 0.95 | intron | PIK3C3 | U |
| 5926 | chr15 | 73547092 | 73547378 | Pancreatic Alpha + Beta + Delta cells | 0.01 | 0.92 | intron | NEO1 | U |
| 5927 | chr1 | 65144855 | 65145174 | Pancreatic Alpha + Beta + Delta cells | 0.03 | 0.93 | intron | CACHD1 | U |
| 5928 | chr4 | 31066890 | 31067290 | Pancreatic Alpha + Beta + Delta cells | 0.01 | 0.91 | intron | PCDH7 | U |
| 5929 | chr4 | 71762250 | 71762512 | Pancreatic Alpha + Beta + Delta cells | 0.04 | 0.91 | Intergenic | MOB1B | U |
| 5930 | chr11 | 11409294 | 11409415 | Pancreatic Alpha + Beta + Delta cells | 0.05 | 0.91 | intron | GALNT18 | U |
| 5931 | chr8 | 30917044 | 30917399 | Pancreatic Alpha + Beta + Delta cells | 0.11 | 0.96 | intron | WRN | U |
| 5932 | chr17 | 17393032 | 17393515 | Pancreatic Alpha + Beta + Delta cells | 0.09 | 0.93 | intron | MED9 | U |
| 5933 | chr6 | 37954346 | 37954445 | Pancreatic Alpha + Beta + Delta cells | 0.11 | 0.95 | intron | ZFAND3 | U |
| 5934 | chr8 | 134312641 | 134312941 | Pancreatic Alpha + Beta + Delta cells | 0.1 | 0.93 | Intergenic | NDRG1 | U |
| 5935 | chr10 | 135144629 | 135144743 | Pancreatic Alpha + Beta + Delta cells | 0.04 | 0.83 | intron | CALY | U |
| 5936 | chr1 | 54317669 | 54317816 | Pancreatic Alpha + Beta + Delta cells | 0.01 | 0.94 | exon | YIPF1 | U |
| 5937 | chr21 | 48072653 | 48072887 | Pancreatic Alpha + Beta + Delta cells | 0.01 | 0.94 | exon | PRMT2 | U |
| 5938 | chr18 | 7994337 | 7994541 | Pancreatic Alpha + Beta + Delta cells | 0.02 | 0.94 | intron | PTPRM | U |
| 5939 | chr1 | 64259663 | 64259887 | Pancreatic Alpha + Beta + Delta cells | 0.01 | 0.93 | intron | ROR1 | U |
| 5940 | chr10 | 114737328 | 114737592 | Pancreatic Alpha + Beta + Delta cells | 0.02 | 0.92 | Intron | TCF7L2 | U |
| 5941 | chr7 | 40132678 | 40132835 | Pancreatic Alpha + Beta + Delta cells | 0.06 | 0.95 | exon | CDK13 | U |
| 5942 | chr2 | 170359492 | 170359676 | Pancreatic Alpha + Beta + Delta cells | 0.04 | 0.93 | intron | BBS5 | U |
| 5943 | chr11 | 66005140 | 66005425 | Pancreatic Alpha + Beta + Delta cells | 0.02 | 0.91 | intron | PACS1 | U |
| 5944 | chr19 | 19123447 | 19123596 | Pancreatic Alpha + Beta + Delta cells | 0.03 | 0.91 | intron | SUGP2 | U |
| 5945 | chr2 | 205584910 | 205585063 | Pancreatic Alpha + Beta + Delta cells | 0.02 | 0.9 | intron | PARD3B | U |
| 5946 | chr13 | 26322725 | 26322920 | Pancreatic Alpha + Beta + Delta cells | 0.01 | 0.89 | intron | ATP8A2 | U |
| 5947 | chr4 | 186297020 | 186297240 | Pancreatic Alpha + Beta + Delta cells | 0.05 | 0.93 | intron | LRP2BP | U |
| 5948 | chr12 | 114336393 | 114336715 | Pancreatic Alpha + Beta + Delta cells | 0.02 | 0.9 | intron | RBM19 | U |
| 5949 | chr3 | 45508353 | 45508521 | Pancreatic Alpha + Beta + Delta cells | 0.03 | 0.9 | intron | LARS2 | U |
| 5950 | chr1 | 12333781 | 12334137 | Pancreatic Alpha + Beta + Delta cells | 0.06 | 0.93 | intron | VPS13D | U |
| 5951 | chr7 | 158165892 | 158165912 | Pancreatic Alpha + Beta + Delta cells | 0.04 | 0.9 | intron | PTPRN2 | U |
| 5952 | chr4 | 78121467 | 78121701 | Pancreatic Alpha + Beta + Delta cells | 0.05 | 0.91 | Intergenic | CCNG2 | U |
| 5953 | chr19 | 5940287 | 5940525 | Pancreatic Alpha + Beta + Delta cells | 0.05 | 0.91 | intron | RANBP3 | U |
| 5954 | chr16 | 20792346 | 20792608 | Pancreatic Alpha + Beta + Delta cells | 0.01 | 0.87 | intron | ACSM3 | U |
| 5955 | chr5 | 126422538 | 126422885 | Pancreatic Alpha + Beta + Delta cells | 0.06 | 0.92 | Intergenic | C5orf63 | U |
| 5956 | chr11 | 1341139341 | 134114344 | Pancreatic Alpha + Beta + Delta cells | 0.05 | 0.91 | intron | VPS26B | U |
| 5957 | chr10 | 34587539 | 34587644 | Pancreatic Alpha + Beta + Delta cells | 0.08 | 0.93 | intron | PARD3 | U |
| 5958 | chr5 | 42828307 | 42828443 | Pancreatic Alpha + Beta + Delta cells | 0.07 | 0.92 | intron | GLTSCR1L | U |
| 5959 | chr2 | 240192453 | 240192609 | Pancreatic Alpha + Beta + Delta cells | 0.02 | 0.87 | intron | HDAC4 | U |
| 5960 | chr16 | 421807 | 421999 | Pancreatic Alpha + Beta + Delta cells | 0.02 | 0.87 | exon | TMEM8A | U |
| 5961 | chr1 | 28178026 | 28178380 | Pancreatic Alpha + Beta + Delta cells | 0.07 | 0.92 | TTS | PPP1R8 | U |
| 5962 | chr5 | 58168362 | 58168815 | Pancreatic Alpha + Beta + Delta cells | 0.04 | 0.89 | Intergenic | RAB3C | U |
| 5963 | chr12 | 84198025 | 84198479 | Pancreatic Alpha + Beta + Delta cells | 0.04 | 0.89 | Intergenic | SLC6A15 | U |
| 5964 | chr12 | 20492456 | 20492590 | Pancreatic Alpha + Beta + Delta cells | 0.03 | 0.87 | Intergenic | PDE3A | U |
| 5965 | chr16 | 16833433 | 1683497 | Pancreatic Alpha + Beta + Delta cells | 0.08 | 0.92 | intron | CRAMP1L | U |
| 5966 | chr12 | 131280340 | 131280618 | Pancreatic Alpha + Beta + Delta cells | 0.04 | 0.88 | intron | STX2 | U |
| 5967 | chr6 | 1178678383 | 117868143 | Pancreatic Alpha + Beta + Delta cells | 0.02 | 0.86 | intron | DCBLD1 | U |
| 5968 | chr2 | 118705617 | 118706081 | Pancreatic Alpha + Beta + Delta cells | 0.1 | 0.94 | intron | CCDC92 | U |
| 5969 | chr3 | 1307961593 | 130796346 | Pancreatic Alpha + Beta + Delta cells | 0.03 | 0.86 | intron | NEK11 | U |
| 5970 | chr8 | 17181976 | 17182233 | Pancreatic Alpha + Beta + Delta cells | 0.09 | 0.92 | intron | MTMR7 | U |
| 5971 | chr18 | 54594071 | 54594535 | Pancreatic Alpha + Beta + Delta cells | 0.05 | 0.88 | intron | WDR7 | U |
| 5972 | chr20 | 1155772 | 1155870 | Pancreatic Alpha + Beta + Delta cells | 0.1 | 0.92 | Intergenic | TMEM74B | U |
| 5973 | chr10 | 1099540631 | 109954170 | Pancreatic Alpha + Beta + Delta cells | 0.02 | 0.84 | Intergenic | RNU6-53 | U |
| 5974 | chr21 | 480694033 | 48069616 | Pancreatic Alpha + Beta + Delta cells | 0.11 | 0.93 | intron, exon | PRMT2, PRMT2 | U |
| 5975 | chr3 | 42157546 | 42157789 | Pancreatic Alpha + Beta + Delta cells | 0.09 | 0.91 | intron | TRAK1 | U |
| 5976 | chr17 | 28537689 | 28537933 | Pancreatic Alpha + Beta + Delta cells | 0.06 | 0.88 | exon, intron | SLC6A4.SLC6A4 | U |
| 5977 | chr15 | 52962514 | 52962777 | Pancreatic Alpha + Beta + Delta cells | 0.07 | 0.89 | intron | FAM214A | U |
| 5978 | chr15 | 89086735 | 89087031 | Pancreatic Alpha + Beta + Delta cells | 0.07 | 0.89 | intron | DET1 | U |
| 5979 | chr2 | 131483184 | 131483493 | Pancreatic Alpha + Beta + Delta cells | 0.02 | 0.84 | Intergenic | GPR148 | U |
| 5980 | chr14 | 1039215493 | 103921880 | Pancreatic Alpha + Beta + Delta cells | 0.1 | 0.92 | intron | MARK3 | U |
| 5981 | chr4 | 261326491 | 26133010 | Pancreatic Alpha + Beta + Delta cells | 0.05 | 0.87 | Intergenic | RBPJ | U |
| 5982 | chr1 | 55549922 | 55550082 | Pancreatic Alpha + Beta + Delta cells | 0.14 | 0.95 | intron | USP24 | U |
| 5983 | chr5 | 133135556 | 133135720 | Pancreatic Alpha + Beta + Delta cells | 0 | 0.81 | Intergenic | C5orf15 | U |
| 5984 | chr10 | 32399766 | 32399985 | Pancreatic Alpha + Beta + Delta cells | 0.1 | 0.91 | Intergenic | KIF58 | U |
| 5985 | chr17 | 43090995 | 43091250 | Pancreatic Alpha + Beta + Delta cells | 0.02 | 0.83 | Intergenic | C1QL1 | U |
| 5986 | chr2 | 192584628 | 192584964 | Pancreatic Alpha + Beta + Delta cells | 0.11 | 0.92 | Intergenic | NABP1 | U |
| 5987 | chr8 | 141166206 | 141166703 | Pancreatic Alpha + Beta + Delta cells | 0.11 | 0.92 | intron | TRAPPC9 | U |
| 5988 | chr17 | 57483062 | 57483164 | Pancreatic Alpha + Beta + Delta cells | 0.12 | 0.92 | Intergenic | MIR4729 | U |
| 5989 | chr10 | 5676954 | 5677161 | Pancreatic Alpha + Beta + Delta cells | 0.04 | 0.84 | Intergenic | ASB13 | U |
| 5990 | chr10 | 97043316 | 97043625 | Pancreatic Alpha + Beta + Delta cells | 0.11 | 0.91 | intron | PDLIM1 | U |
| 5991 | chr5 | 179044384 | 179044640 | Pancreatic Alpha + Beta + Delta cells | 0.14 | 0.93 | intron | HNRNPH1 | U |
| 5992 | chr5 | 176357690 | 176357984 | Pancreatic Alpha + Beta + Delta cells | 0.07 | 0.86 | intron | UIMC1 | U |
| 5993 | chr4 | 79747213 | 79747251 | Pancreatic Alpha + Beta + Delta cells | 0.18 | 0.96 | exon | BMP2K | U |
| 5994 | chr10 | 135144559 | 135144744 | Pancreatic Alpha + Beta + Delta cells | 0.06 | 0.84 | intron | CALY | U |
| 5995 | chr15 | 393237461 | 39323965 | Pancreatic Alpha + Beta + Delta cells | 0.02 | 0.8 | Intergenic | C15orf54 | U |
| 5996 | chr19 | 137110813 | 13711310 | Pancreatic Alpha + Beta + Delta cells | 0.04 | 0.82 | Intergenic | CACNA1A | U |
| 5997 | chr19 | 13710721 | 13710987 | Pancreatic Alpha + Beta + Delta cells | 0.02 | 0.8 | Intergenic | CACNA1A | U |
| 5998 | chr8 | 141339511 | 141339794 | Pancreatic Alpha + Beta + Delta cells | 0.15 | 0.93 | intron | TRAPPC9 | U |
| 5999 | chr1 | 2331407811 | 233141129 | Pancreatic Alpha + Beta + Delta cells | 0.14 | 0.92 | intron | PCNXL2 | U |
| 6000 | chr1 | 96295091 | 9629893 | Pancreatic Alpha + Beta + Delta cells | 0.12 | 0.9 | intron | SLC25A33 | U |
| 6001 | chr1 | 90181842 | 90182263 | Pancreatic Alpha + Beta + Delta cells | 0.16 | 0.94 | exon | LRRC8C | U |
| 6002 | chr3 | 4482756 | 4482874 | Pancreatic Alpha + Beta + Delta cells | 0.1 | 0.87 | intron | SUMF1 | U |
| 6003 | chr10 | 1042817581 | 104281988 | Pancreatic Alpha + Beta + Delta cells | 0.18 | 0.94 | intron | SUFU | U |
| 6004 | chr7 | 1877468 | 1877625 | Pancreatic Alpha + Beta + Delta cells | 0.19 | 0.94 | intron | MAD1L1 | U |
| 6005 | chr7 | 4175336 | 4175436 | Pancreatic Alpha + Beta + Delta cells | 0.03 | 0.77 | intron | SDK1 | U |
| 6006 | chr3 | 731878763 | 73188112 | Pancreatic Alpha + Beta + Delta cells | 0.15 | 0.89 | Intergenic | EBLN2 | U |
| 6007 | chr9 | 5655518 | 5655756 | Pancreatic Alpha + Beta + Delta cells | 0.21 | 0.94 | intron | KIAA1432 | U |
| 6008 | chr3 | 169880832 | 169881092 | Pancreatic Alpha + Beta + Delta cells | 0.24 | 0.95 | intron | PHC3 | U |
| 6009 | chr4 | 156609240 | 156609367 | Pancreatic Alpha + Beta + Delta cells | 0.23 | 0.93 | intron | GUCY1A3 | U |
| 6010 | chr2 | 73534707 | 73534951 | Pancreatic Alpha + Beta + Delta cells | 0.14 | 0.83 | Intergenic | EGR4 | U |
| 6011 | chr4 | 245450701 | 24545323 | Pancreatic Alpha + Beta + Delta cells | 0.26 | 0.92 | intron | DHX15 | U |
| 6012 | chr6 | 1516099523 | 151610147 | Pancreatic Alpha + Beta + Delta cells | 0.01 | 0.91 | intron | AKAP12 | U |
| 6013 | chr7 | 38479445 | 38479649 | Pancreatic Alpha + Beta + Delta cells | 0.02 | 0.88 | intron | AMPH | U |
| 6014 | chr7 | 37149874 | 37150285 | Pancreatic Alpha + Beta + Delta cells | 0.08 | 0.85 | intron | ELMO1 | U |
| 6015 | chr18 | 77076138 | 77076400 | Pancreatic Alpha + Beta + Delta cells | 0.02 | 0.96 | intron | ATP9B | U |
| 6016 | chr4 | 184205408 | 184205523 | Pancreatic Alpha + Beta + Delta cells | 0.02 | 0.93 | exon | WWC2 | U |
| 6017 | chr14 | 102467285 | 102467421 | Pancreatic Alpha + Beta + Delta cells | 0.02 | 0.93 | exon | DYNC1H1 | U |
| 6018 | chr6 | 35196350 | 35196500 | Pancreatic Alpha + Beta + Delta cells | 0.03 | 0.94 | exon | SCUBE3 | U |
| 6019 | chr19 | 5080780 | 5080861 | Pancreatic Alpha + Beta + Delta cells | 0.02 | 0.92 | intron | KDM48 | U |
| 6020 | chr2 | 3425344 | 3425499 | Pancreatic Alpha + Beta + Delta cells | 0.05 | 0.94 | intron | TRAPPC12 | U |
| 6021 | chr17 | 29669584 | 29670054 | Pancreatic Alpha + Beta + Delta cells | 0.06 | 0.95 | intron | NF1 | U |
| 6022 | chr19 | 50424333 | 50424506 | Pancreatic Alpha + Beta + Delta cells | 0.07 | 0.92 | intron | NUP62 | U |
| 6023 | chr1 | 110560854 | 110561213 | Pancreatic Alpha + Beta + Delta cells | 0.09 | 0.94 | exon | AHCYL1 | U |
| 6024 | chr6 | 7565596 | 7565819 | Pancreatic Alpha + Beta + Delta cells | 0.11 | 0.92 | exon | DSP | U |
| 6025 | chr6 | 26863547 | 26863775 | Pancreatic Alpha + Beta + Delta cells | 0.12 | 0.92 | intron | GUSBP2 | U |
| 6026 | chr14 | 77171592 | 77172087 | Pancreatic Alpha + Beta + Delta cells | 0.09 | 0.89 | Intergenic | VASH1 | U |
| 6027 | chr18 | 43450431 | 43450905 | Pancreatic Alpha + Beta + Delta cells | 0.03 | 0.93 | intron | EPG5 | U |
| 6028 | chr19 | 14092942 | 14093277 | Pancreatic Alpha + Beta + Delta cells | 0.04 | 0.93 | intron | RFX1 | U |
| 6029 | chr14 | 66533815 | 66533930 | Pancreatic Alpha + Beta + Delta cells | 0.06 | 0.94 | Intergenic | LINC00238 | U |
| 6030 | chr20 | 48446527 | 48446742 | Pancreatic Alpha + Beta + Delta cells | 0.06 | 0.94 | intron | SLC9A8 | U |
| 6031 | chr10 | 77358893 | 77359172 | Pancreatic Alpha + Beta + Delta cells | 0.02 | 0.9 | Intergenic | C10orf11 | U |
| 6032 | chr9 | 97939019 | 97939398 | Pancreatic Alpha + Beta + Delta cells | 0.05 | 0.93 | intron | FANCC | U |
| 6033 | chr20 | 5819299 | 5819411 | Pancreatic Alpha + Beta + Delta cells | 0.04 | 0.91 | intron | C20orf196 | U |
| 6034 | chr11 | 36297713 | 36298011 | Pancreatic Alpha + Beta + Delta cells | 0.09 | 0.94 | intron | COMMD9 | U |
| 6035 | chr15 | 99340557 | 99340966 | Pancreatic Alpha + Beta + Delta cells | 0.03 | 0.88 | intron | IGFIR | U |
| 6036 | chr4 | 7035875 | 7035989 | Pancreatic Alpha + Beta + Delta cells | 0.07 | 0.91 | intron | LOC100129931 | U |
| 6037 | chr14 | 495159821 | 49516171 | Pancreatic Alpha + Beta + Delta cells | 0.01 | 0.85 | Intergenic | RPS29 | U |
| 6038 | chr2 | 537435303 | 53743727 | Pancreatic Alpha + Beta + Delta cells | 0.05 | 0.89 | Intergenic | CHAC2 | U |
| 6039 | chr6 | 15358643 | 15359092 | Pancreatic Alpha + Beta + Delta cells | 0.06 | 0.89 | intron | JARID2 | U |
| 6040 | chr12 | 114318713 | 114319202 | Pancreatic Alpha + Beta + Delta cells | 0.04 | 0.86 | intron | RBM19 | U |
| 6041 | chr14 | 69160412 | 69160600 | Pancreatic Alpha + Beta + Delta cells | 0.08 | 0.89 | Intergenic | ZFP36L1 | U |
| 6042 | chr6 | 7463279 | 7463590 | Pancreatic Alpha + Beta + Delta cells | 0.11. | 0.92 | Intergenic | RIOK1 | U |
| 6043 | chr14 | 57896917 | 57897246 | Pancreatic Alpha + Beta + Delta cells | 0.07 | 0.88 | Intergenic | NAA30 | U |
| 6044 | chr7 | 5316237 | 5316354 | Pancreatic Alpha + Beta + Delta cells | 0.02 | 0.81 | Intergenic | SLC29A4 | U |
| 6045 | chr9 | 16527607 | 16527950 | Pancreatic Alpha + Beta + Delta cells | 0.08 | 0.87 | intron | BNC2 | U |
| 6046 | chr14 | 56759745 | 56760150 | Pancreatic Alpha + Beta + Delta cells | 0.11 | 0.9 | intron | PELI2 | U |
| 6047 | chr19 | 50423947 | 50424127 | Pancreatic Alpha + Beta + Delta cells | 0.21 | 0.95 | intron | NUP62 | U |
| 6048 | chr6 | 43749236 | 43749716 | Pancreatic Alpha + Beta + Delta cells | 0.19 | 0.91 | intron | VEGFA | U |
| 6049 | chr11 | 68210149 | 68210383 | Pancreatic Alpha + Beta + Delta cells | 0.2 | 0.9 | intron | LRP5 | U |
| 6050 | chr12 | 132504671 | 132505052 | Pancreatic Alpha + Beta + Delta cells | 0.32 | 0.96 | exon, intron | EP400, EP400 | U |
| 6051 | chr1 | 1563844301 | 156384531 | Pancreatic Alpha + Beta + Delta cells | 0.89 | 0.09 | exon | C1orf61 | M |
| 6052 | chr17 | 27331275 | 27331446 | Pancreatic Alpha + Beta + Delta cells | 0.98 | 0.19 | intron | SEZ6 | M |
| 6053 | chr7 | 1568160663 | 156816343 | Pancreatic Alpha + Beta + Delta cells | 0.89 | 0.12 | Intergenic | LOC645249 | M |
| 6054 | chr14 | 93369148 | 93369285 | Pancreatic Alpha + Beta + Delta cells | 0.87 | 0.12 | Intergenic | CHGA | M |
| 6055 | chr17 | 48642299 | 48642486 | Pancreatic Alpha + Beta + Delta cells | 0.91 | 0.17 | intron | CACNA1G | M |
| 6056 | chr17 | 48610850 | 48611055 | Pancreatic Alpha + Beta + Delta cells | 0.81 | 0.14 | exon, intron | EPN3, EPN3 | M |
| 6057 | chr14 | 93369380 | 93369468 | Pancreatic Alpha + Beta + Delta cells | 0.78 | 0.16 | Intergenic | CHGA | M |
| 6058 | chr2 | 2201965303 | 220196662 | Pancreatic Alpha + Beta + Delta cells | 0.94 | 0.09 | intron | RESP18 | M |
| 6059 | chr2 | 220196370 | 220196475 | Pancreatic Alpha + Beta + Delta cells | 0.83 | 0.04 | intron | RESP18 | M |
| 6060 | chr7 | 1568154783 | 156815807 | Pancreatic Alpha + Beta + Delta cells | 0.87 | 0.09 | Intergenic | LOC645249 | M |
| 6061 | chr7 | 127808701 | 127808805 | Pancreatic Alpha + Beta + Delta cells | 0.83 | 0.06 | Intergenic | MIR129-1 | M |
| 6062 | chr13 | 28541256 | 28541563 | Pancreatic Alpha + Beta + Delta cells | 0.88 | 0.11 | intron | CDX2 | M |
| 6063 | chr6 | 41472431 | 41472728 | Pancreatic Alpha + Beta + Delta cells | 0.84 | 0.08 | Intergenic | FOXP4 | M |
| 6064 | chr19 | 13618495 | 13618839 | Pancreatic Alpha + Beta + Delta cells | 0.86 | 0.1 | promoter-TSS, Interg [symbol] ; | CACNA1A, CACNA1A | M |
| 6065 | chr19 | 591536 | 591781 | Pancreatic Alpha + Beta + Delta cells | 0.78 | 0.03 | intron | HON2 | M |
| 6066 | chr13 | 28541611 | 28542053 | Pancreatic Alpha + Beta + Delta cells | 0.85 | 0.1 | intron | CDX2 | M |
| 6067 | chr2 | 73518221 | 73518240 | Pancreatic Alpha + Beta + Delta cells | 0.95 | 0.23 | exon | EGR4 | M |
| 6068 | chr6 | 414720321 | 41472207 | Pancreatic Alpha + Beta + Delta cells | 0.8 | 0.11 | Intergenic | FOXP4 | M |
| 6069 | chr10 | 71336088 | 71336385 | Pancreatic Alpha + Beta + Delta cells | 0.75 | 0.06 | Intergenic | NEUROG3 | M |
| 6070 | chr16 | 89988197 | 89988547 | Pancreatic Alpha + Beta + Delta cells | 0.72 | 0.05 | promoter-TSS | TUBB3 | M |
| 6071 | chr8 | 54790614 | 54790842 | Pancreatic Alpha + Beta + Delta cells | 0.7 | 0.05 | intron | RGS20 | M |
| 6072 | chr2 | 73519827 | 73519905 | Pancreatic Alpha + Beta + Delta cells | 0.78 | 0.15 | exon | EGR4 | M |
| 6073 | chr11 | 68607274 | 68607623 | Pancreatic Alpha + Beta + Delta cells | 0.6 | 0.04 | intron | CPT1A | M |
| 6074 | chr2 | 73518277 | 73518558 | Pancreatic Alpha + Beta + Delta cells | 0.57 | 0.02 | exon | EGR4 | M |
| 6075 | chr19 | 13388075 | 13388264 | Pancreatic Alpha + Beta + Delta cells | 0.6 | 0.08 | intron | CACNA1A | M |
| 6076 | chr12 | 279488583 | 27949119 | Breast Basal + Luminal Epithelium | 0.06 | 0.88 | intron | KLHL42 | U |
| 6077 | chr3 | 150491825 | 150491964 | Breast Basal + Luminal Epithelium | 0.12 | 0.94 | Intergenic | SIAH2 | U |
| 6078 | chr8 | 47144675 | 47144709 | Breast Basal + Luminal Epithelium | 0.06 | 0.86 | Intergenic | LINC00293 | U |
| 6079 | chr16 | 1478098 | 1478339 | Breast Basal + Luminal Epithelium | 0.09 | 0.88 | Intergenic | C16orf91 | U |
| 6080 | chr2 | 127240232 | 127240497 | Breast Basal + Luminal Epithelium | 0.13 | 0.92 | Intergenic | GYPC | U |
| 6081 | chr3 | 126775127 | 126775296 | Breast Basal + Luminal Epithelium | 0.14 | 0.92 | Intergenic | PLXNA1 | U |
| 6082 | chr11 | 224125191 | 22412746 | Breast Basal + Luminal Epithelium | 0.07 | 0.85 | Intergenic | SLC17A6 | U |
| 6083 | chr10 | 1193148941 | 119314952 | Breast Basal + Luminal Epithelium | 0.04 | 0.82 | Intergenic | EMX2OS | U |
| 6084 | chr20 | 60395236 | 60395387 | Breast Basal + Luminal Epithelium | 0.07 | 0.84 | intron | CDH4 | U |
| 6085 | chr10 | 19443233 | 19443502 | Breast Basal + Luminal Epithelium | 0.11 | 0.88 | Intergenic | ARL5B | U |
| 6086 | chr13 | 39540888 | 39541095 | Breast Basal + Luminal Epithelium | 0.14 | 0.9 | exon | STOML3 | U |
| 6087 | chr18 | 57090982 | 57091074 | Breast Basal + Luminal Epithelium | 0.15 | 0.9 | Intergenic | LMAN1 | U |
| 6088 | chr8 | 47145602 | 47145697 | Breast Basal + Luminal Epithelium | 0.16 | 0.88 | Intergenic | LINC00293 | U |
| 6089 | chr19 | 200562943 | 20056492 | Breast Basal + Luminal Epithelium | 0.22 | 0.93 | Intergenic | ZNF93 | U |
| 6090 | chr1 | 204406713 | 204407056 | Breast Basal + Luminal Epithelium | 0.15 | 0.86 | intron | PIK3C2B | U |
| 6091 | chr10 | 109305382 | 109305724 | Breast Basal + Luminal Epithelium | 0.1 | 0.93 | Intergenic | SORCS1 | U |
| 6092 | chr1 | 2832006 | 2832144 | Breast Basal + Luminal Epithelium | 0.06 | 0.85 | Intergenic | ACTRT2 | U |
| 6093 | chr2 | 236055974 | 236056170 | Breast Basal + Luminal Epithelium | 0.11 | 0.9 | Intergenic | SH3BP4 | U |
| 6094 | chr7 | 477868833 | 47786991 | Breast Basal + Luminal Epithelium | 0.09 | 0.87 | Intergenic | LINC00525 | U |
| 6095 | chr6 | 777457 | 777756 | Breast Basal + Luminal Epithelium | 0.11 | 0.89 | Intergenic | EXOC2 | U |
| 6096 | chr18 | 76079910 | 76079962 | Breast Basal + Luminal Epithelium | 0.08 | 0.85 | Intergenic | SALL3 | U |
| 6097 | chr14 | 96013460 | 96013940 | Breast Basal + Luminal Epithelium | 0.12 | 0.89 | Intergenic | GLRX5 | U |
| 6098 | chr19 | 28351260 | 28351437 | Breast Basal + Luminal Epithelium | 0.1 | 0.85 | Intergenic | LINC00662 | U |
| 6099 | chr10 | 2271089 | 2271521 | Breast Basal + Luminal Epithelium | 0.06 | 0.81 | Intergenic | LINC00701 | U |
| 6100 | chr2 | 116372189 | 116372271 | Breast Basal + Luminal Epithelium | 0.16 | 0.9 | intron | DPP10 | U |
| 6101 | chr20 | 62012031 | 62012139 | Breast Basal + Luminal Epithelium | 0.06 | 0.8 | Intergenic | CHRNA4 | U |
| 6102 | chr16 | 1477888 | 1478047 | Breast Basal + Luminal Epithelium | 0.12 | 0.86 | Intergenic | C16orf91 | U |
| 6103 | chr1 | 41889792 | 41889967 | Breast Basal + Luminal Epithelium | 0.13 | 0.87 | Intergenic | EDN2 | U |
| 6104 | chr2 | 9289261 | 9289498 | Breast Basal + Luminal Epithelium | 0.17 | 0.91 | Intergenic | ASAP2 | U |
| 6105 | chr2 | 16362594 | 16362687 | Breast Basal + Luminal Epithelium | 0.16 | 0.89 | Intergen | MYCNOS | U |
| 6106 | chr16 | 881470333 | 88147160 | Breast Basal + Luminal Epithelium | 0.15 | 0.88 | Intergenic | BANP | U |
| 6107 | chr11 | 1197923341 | 119792556 | Breast Basal + Luminal Epithelium | 0.12 | 0.85 | Intergenic | PVRL1 | U |
| 6108 | chr5 | 24295521 | 24295713 | Breast Basal + Luminal Epithelium | 0.15 | 0.87 | Intergenic | CDH10 | U |
| 6109 | chr14 | 95462015 | 95462278 | Breast Basal + Luminal Epithelium | 0.12 | 0.84 | Intergenic | MIR3173 | U |
| 6110 | chr16 | 1478441 | 1478505 | Breast Basal + Luminal Epithelium | 0.1 | 0.81 | Intergenic | C16orf91 | U |
| 6111 | chr12 | 132984025 | 132984098 | Breast Basal + Luminal Epithelium | 0.1 | 0.81 | Intergenic | FBRSL1 | U |
| 6112 | chr2 | 118134453 | 118134531 | Breast Basal + Luminal Epithelium | 0.18 | 0.89 | Intergenic | DDX18 | U |
| 6113 | chr5 | 4340164 | 4340279 | Breast Basal + Luminal Epithelium | 0.12 | 0.83 | Intergenic | LOC340094 | U |
| 6114 | chr5 | 177513083 | 177513285 | Breast Basal + Luminal Epithelium | 0.12 | 0.83 | Intergenic | N4BP3 | U |
| 6115 | chr18 | 76080046 | 76080295 | Breast Basal + Luminal Epithelium | 0.12 | 0.83 | Intergenic | SALL3 | U |
| 6116 | chr8 | 23227916 | 23228187 | Breast Basal + Luminal Epithelium | 0.18 | 0.89 | intron | LOXL2 | U |
| 6117 | chr11 | 117928221 | 117928533 | Breast Basal + Luminal Epithelium | 0.12 | 0.83 | intron | TMPRSS4-AS1 | U |
| 6118 | chr9 | 9559677 | 9560036 | Breast Basal + Luminal Epithelium | 0.17 | 0.88 | intron | PTPRD | U |
| 6119 | chr8 | 1443181701 | 144318316 | Breast Basal + Luminal Epithelium | 0.18 | 0.88 | Intergenic | ZFP41 | U |
| 6120 | chr6 | 120792972 | 120793264 | Breast Basal + Luminal Epithelium | 0.2 | 0.9 | Intergenic | C6orf170 | U |
| 6121 | chr20 | 620448241 | 62044927 | Breast Basal + Luminal Epithelium | 0.12 | 0.81 | exon | KCNQ2 | U |
| 6122 | chr20 | 55277556 | 55277677 | Breast Basal + Luminal Epithelium | 0.1 | 0.79 | Intergenic | TFAP2C | U |
| 6123 | chr10 | 129333736 | 129334007 | Breast Basal + Luminal Epithelium | 0.15 | 0.84 | Intergenic | NPS | U |
| 6124 | chr13 | 45956569 | 45956926 | Breast Basal + Luminal Epithelium | 0.17 | 0.85 | intron | TPT1-AS1 | U |
| 6125 | chr1 | 187261133 | 187261541 | Breast Basal + Luminal Epithelium | 0.18 | 0.86 | Intergenic | PLA2G4A | U |
| 6126 | chr8 | 60347041 | 60347495 | Breast Basal + Luminal Epithelium | 0.18 | 0.86 | Intergenic | TOX | U |
| 6127 | chr8 | 144209697 | 144209937 | Breast Basal + Luminal Epithelium | 0.16 | 0.83 | Intergenic | LY6H | U |
| 6128 | chr4 | 7158963 | 7159206 | Breast Basal + Luminal Epithelium | 0.22 | 0.89 | Intergenic | SORCS2 | U |
| 6129 | chr16 | 30635481 | 30635748 | Breast Basal + Luminal Epithelium | 0.22 | 0.89 | Intergenic | ZNF689 | U |
| 6130 | chr1 | 4177851 | 4177902 | Breast Basal + Luminal Epithelium | 0.16 | 0.82 | Intergenic | LOC728716 | U |
| 6131 | chrX | 69873831 | 6987528 | Breast Basal + Luminal Epithelium | 0.22 | 0.88 | intron | HDHD1 | U |
| 6132 | chr1 | 224638084 | 224638508 | Breast Basal + Luminal Epithelium | 0.22 | 0.88 | Intergenic | WDR26 | U |
| 6133 | chr20 | 547817211 | 54781848 | Breast Basal + Luminal Epithelium | 0.21 | 0.86 | Intergenic | MC3R | U |
| 6134 | chr1 | 5897580 | 5897790 | Breast Basal + Luminal Epithelium | 0.22 | 0.87 | Intergenic | MIR4689 | U |
| 6135 | chr19 | 13363864 | 13364081 | Breast Basal + Luminal Epithelium | 0.16 | 0.81 | exon, intron | CACNA1A, CACNA1A | U |
| 6136 | chr4 | 6973417 | 6973673 | Breast Basal + Luminal Epithelium | 0.25 | 0.9 | intron | TBC1D14 | U |
| 6137 | chr12 | 118210555 | 118210833 | Breast Basal + Luminal Epithelium | 0.16 | 0.81 | intron | KSR2 | U |
| 6138 | chr4 | 3739508 | 3739600 | Breast Basal + Luminal Epithelium | 0.16 | 0.79 | Intergenic | ADRA2C | U |
| 6139 | chr19 | 469682741 | 46968393 | Breast Basal + Luminal Epithelium | 0.22 | 0.85 | Intergenic | PNMAL1 | U |
| 6140 | chr16 | 3723608 | 3723868 | Breast Basal + Luminal Epithelium | 0.26 | 0.89 | intron | TRAP1 | U |
| 6141 | chr10 | 12148206 | 121482349 | Breast Basal + Luminal Epithelium | 0.22 | 0.85 | Intergenic | INPP5F | U |
| 6142 | chr10 | 1301205791 | 130120877 | Breast Basal + Luminal Epithelium | 0.18 | 0.81 | Intergenic | MKI67 | U |
| 6143 | chr13 | 95204863 | 95205265 | Breast Basal + Luminal Epithelium | 0.28 | 0.91 | Intergenic | TGDS | U |
| 6144 | chr12 | 123211652 | 123212092 | Breast Basal + Luminal Epithelium | 0.25 | 0.88 | TTS | HCAR1 | U |
| 6145 | chr10 | 132777726 | 132777845 | Breast Basal + Luminal Epithelium | 0.3 | 0.92 | Intergenic | MIR378C | U |
| 6146 | chr9 | 137721879 | 137722012 | Breast Basal + Luminal Epithelium | 0.26 | 0.88 | intron | COLSA1 | U |
| 6147 | chr8 | 1443183381 | 144318534 | Breast Basal + Luminal Epithelium | 0.17 | 0.79 | Intergenic | ZFP41 | U |
| 6148 | chr13 | 221998703 | 22200074 | Breast Basal + Luminal Epithelium | 0.18 | 0.8 | Intergenic | EFHA1 | U |
| 6149 | chr10 | 134955754 | 134955800 | Breast Basal + Luminal Epithelium | 0.22 | 0.82 | Intergenic | KNDC1 | U |
| 6150 | chr5 | 177943384 | 177943499 | Breast Basal + Luminal Epithelium | 0.24 | 0.83 | intron | COL23A1 | U |
| 6151 | chr13 | 112298627 | 112298747 | Breast Basal + Luminal Epithelium | 0.24 | 0.83 | Intergenic | TEX29 | U |
| 6152 | chr12 | 124362406 | 124362551 | Breast Basal + Luminal Epithelium | 0.22 | 0.81 | intron | DNAH10 | U |
| 6153 | chr10 | 126955448 | 126955719 | Breast Basal + Luminal Epithelium | 0.24 | 0.81 | Intergenic | MIR4296 | U |
| 6154 | chr4 | 42110492 | 42110914 | Breast Basal + Luminal Epithelium | 0.26 | 0.83 | Intergenic | BEND4 | U |
| 6155 | chr16 | 1166174 | 1166448 | Breast Basal + Luminal Epithelium | 0.24 | 0.8 | Intergenic | C1QTNF8 | U |
| 6156 | chr8 | 142949835 | 142949969 | Breast Basal + Luminal Epithelium | 0.24 | 0.78 | Intergenic | MIR4472-1 | U |
| 6157 | chr20 | 61320511 | 61320683 | Breast Basal + Luminal Epithelium | 0.32 | 0.86 | Intergenic | NTSR1 | U |
| 6158 | chr4 | 127123265 | 127123416 | Breast Basal + Luminal Epithelium | 0.26 | 0.79 | Intergenic | MIR2054 | U |
| 6159 | chr16 | 1164680 | 1164798 | Breast Basal + Luminal Epithelium | 0.32 | 0.84 | Intergenic | C1QTNF8 | U |
| 6160 | chr7 | 37152662 | 37152918 | Breast Basal + Luminal Epithelium | 0.03 | 0.9 | intron | ELMO1 | U |
| 6161 | chr9 | 122025916 | 122026182 | Breast Basal + Luminal Epithelium | 0.26 | 0.88 | intron | DBC1 | U |
| 6162 | chr10 | 8111648 | 8112126 | Breast Basal + Luminal Epithelium | 0.19 | 0.8 | intron | GATA3 | U |
| 6163 | chr22 | 23801402 | 23801638 | Breast Basal + Luminal Epithelium | 0.03 | 0.92 | Intergenic | ZDHHC8P1 | U |
| 6164 | chr2 | 36668427 | 36668651 | Breast Basal + Luminal Epithelium | 0.06 | 0.89 | exon | CRIM1 | U |
| 6165 | chr1 | 228083415 | 228083606 | Breast Basal + Luminal Epithelium | 0.08 | 0.88 | Intergenic | MIR5008 | U |
| 6166 | chr11 | 232603 | 233048 | Breast Basal + Luminal Epithelium | 0.18 | 0.94 | intron | SIRT3 | U |
| 6167 | chr2 | 265246883 | 26524871 | Breast Basal + Luminal Epithelium | 0.19 | 0.94 | Intergenic | EPT1 | U |
| 6168 | chr11 | 233160 | 233418 | Breast Basal + Luminal Epithelium | 0.14 | 0.89 | intron | SIRT3 | U |
| 6169 | chr16 | 4787695 | 4787972 | Breast Basal + Luminal Epithelium | 0.11 | 0.83 | exon, intron | C16orf71 | U |
| 6170 | chr2 | 242757513 | 242757606 | Breast Basal + Luminal Epithelium | 0.22 | 0.91 | exon | NEU4 | U |
| 6171 | chr5 | 159776351 | 159776480 | Breast Basal + Luminal Epithelium | 0.27 | 0.94 | exon | C1QTNF2 | U |
| 6172 | chr12 | 4737521 | 4737961 | Breast Basal + Luminal Epithelium | 0.09 | 0.88 | exon | AKAP3 | U |
| 6173 | chr14 | 416136201 | 41613685 | Breast Basal + Luminal Epithelium | 0.16 | 0.92 | Intergenic | LRFN5 | U |
| 6174 | chr9 | 78432781 | 78433095 | Breast Basal + Luminal Epithelium | 0.06 | 0.82 | Intergenic | PCSK5 | U |
| 6175 | chr17 | 2278727 | 2279131 | Breast Basal + Luminal Epithelium | 0.14 | 0.9 | exon, intron | SG5M2, SGSM2 | U |
| 6176 | chr5 | 5571530 | 5571829 | Breast Basal + Luminal Epithelium | 0.12 | 0.86 | Intergenic | KIAA0947 | U |
| 6177 | chr5 | 177525862 | 177525915 | Breast Basal + Luminal Epithelium | 0.17 | 0.89 | Intergenic | N4BP3 | U |
| 6178 | chr20 | 62011905 | 62012017 | Breast Basal + Luminal Epithelium | 0.18 | 0.9 | Intergenic | CHRNA4 | U |
| 6179 | chr14 | 28626361 | 28626746 | Breast Basal + Luminal Epithelium | 0.16 | 0.87 | Intergenic | LINC00645 | U |
| 6180 | chr2 | 242757430 | 242757494 | Breast Basal + Luminal Epithelium | 0.14 | 0.84 | exon | NEU4 | U |
| 6181 | chr9 | 126154260 | 126154352 | Breast Basal + Luminal Epithelium | 0.14 | 0.84 | intron | DENND1A | U |
| 6182 | chr2 | 44497562 | 44497826 | Breast Basal + Luminal Epithelium | 0.24 | 0.94 | Intergenic | SLC3A1 | U |
| 6183 | chr6 | 169641078 | 169641365 | Breast Basal + Luminal Epithelium | 0.19 | 0.89 | intron | THBS2 | U |
| 6184 | chr9 | 126154361 | 126154758 | Breast Basal + Luminal Epithelium | 0.2 | 0.9 | intron | DENND1A | U |
| 6185 | chr21 | 46886593 | 46886690 | Breast Basal + Luminal Epithelium | 0.22 | 0.89 | intron | COL18A1 | U |
| 6186 | chr14 | 41888904 | 41889153 | Breast Basal + Luminal Epithelium | 0.15 | 0.82 | Intergenic | LRFN5 | U |
| 6187 | chr8 | 144209496 | 144209612 | Breast Basal + Luminal Epithelium | 0.11 | 0.77 | Intergenic | LY6H | U |
| 6188 | chr3 | 185565216 | 185565377 | Breast Basal + Luminal Epithelium | 0.24 | 0.89 | Intergenic | IGF28PZ | U |
| 6189 | chr22 | 34573021 | 34573101 | Breast Basal + Luminal Epithelium | 0.26 | 0.89 | Intergenic | LARGE | U |
| 6190 | chr20 | 31144203 | 31144462 | Breast Basal + Luminal Epithelium | 0.25 | 0.87 | intron | C20orf112 | U |
| 6191 | chr1 | 6133736 | 6134181 | Breast Basal + Luminal Epithelium | 0.26 | 0.88 | intron | KCNAB2 | U |
| 6192 | chr12 | 124788301 | 124788723 | Breast Basal + Luminal Epithelium | 0.28 | 0.89 | intron | FAM101A | U |
| 6193 | chr8 | 143334441 | 143334602 | Breast Basal + Luminal Epithelium | 0.23 | 0.83 | intron | TSNARE1 | U |
| 6194 | chr1 | 2811624 | 2811803 | Breast Basal + Luminal Epithelium | 0.3 | 0.9 | Intergenic | TTC34 | U |
| 6195 | chr7 | 502956 | 503264 | Breast Basal + Luminal Epithelium | 0.32 | 0.92 | Intergenic | PDGFA | U |
| 6196 | chr22 | 49580759 | 49580874 | Breast Basal + Luminal Epithelium | 0.28 | 0.87 | Intergenic | LOC100128946 | U |
| 6197 | chr20 | 19407815 | 19408159 | Breast Basal + Luminal Epithelium | 0.24 | 0.83 | intron | SLC24A3 | U |
| 6198 | chr10 | 134795381 | 134795424 | Breast Basal + Luminal Epithelium | 0.31 | 0.88 | Intergenic | LOC399829 | U |
| 6199 | chr8 | 58105962 | 58106069 | Breast Basal + Luminal Epithelium | 0.3 | 0.87 | Intergenic | LOC100507651 | U |
| 6200 | chr22 | 17337857 | 17337932 | Breast Basal + Luminal Epithelium | 0.23 | 0.78 | Intergenic | HSFY1P1 | U |
| 6201 | chr2 | 241074519 | 241074658 | Breast Basal + Luminal Epithelium | 0.25 | 0.76 | Intron | MYEOV2 | U |
| 6202 | chr12 | 54433551 | 54433688 | Breast Basal + Luminal Epithelium | 0.86 | 0.1 | intron | HOXC4 | M |
| 6203 | chr3 | 180462104 | 180462290 | Breast Basal + Luminal Epithelium | 0.9 | 0.18 | Intergenic | CCDC39 | M |
| 6204 | chr7 | 87935935 | 87935985 | Breast Basal + Luminal Epithelium | 0.64 | 0.09 | intron | STEAP4 | M |
| 6205 | chr19 | 46580060 | 46580218 | Breast Basal + Luminal Epithelium | 0.68 | 0.13 | Intergenic | IGFL4 | M |
| 6206 | chrX | 103812854 | 103813216 | Breast Basal + Luminal Epithelium | 0.83 | 0.29 | intron | IL1RAPL2 | M |
| 6207 | chr11 | 61062962 | 61063044 | Breast Basal + Luminal Epithelium | 0.92 | 0.1 | promoter-TSS | VWCE | M |
| 6208 | chr5 | 187552 | 1875940 | Breast Basal + Luminal Epithelium | 0.88 | 0.09 | Intergenic | IRX4 | M |
| 6209 | chr2 | 133428731 | 133428800 | Breast Basal + Luminal Epithelium | 0.93 | 0.18 | promoter-TSS | LYPD1 | M |
| 6210 | chr12 | 16757947 | 16758268 | Breast Basal + Luminal Epithelium | 0.82 | 0.08 | exon, promoter-TSS | LMO3, LMO3 | M |
| 6211 | chr3 | 9904307 | 9904634 | Breast Basal + Luminal Epithelium | 0.8 | 0.06 | Intergenic | CIDEC | M |
| 6212 | chr12 | 54408233 | 54408284 | Breast Basal + Luminal Epithelium | 0.82 | 0.09 | Intergenic | HOXC6 | M |
| 6213 | chr12 | 16757837 | 16757945 | Breast Basal + Luminal Epithelium | 0.94 | 0.22 | exon | LMO3 | M |
| 6214 | chr4 | 102712216 | 102712398 | Breast Basal + Luminal Epithelium | 0.8 | 0.1 | intron | BANK1 | M |
| 6215 | chr11 | 61062685 | 61062936 | Breast Basal + Luminal Epithelium | 0.72 | 0.02 | promoter-TSS | VWCE | M |
| 6216 | chr10 | 98479658 | 98479744 | Breast Basal + Luminal Epithelium | 0.82 | 0.13 | intron | PIK3AP1 | M |
| 6217 | chr6 | 3053739 | 3053881 | Breast Basal + Luminal Epithelium | 0.8 | 0.11 | Intergenic | RIPK1 | M |
| 6218 | chr12 | 49390678 | 49391080 | Breast Basal + Luminal Epithelium | 0.74 | 0.05 | exon | DDN | M |
| 6219 | chr18 | 12253405 | 12253640 | Breast Basal + Luminal Epithelium | 0.82 | 0.14 | promoter-TSS | CIDEA | M |
| 6220 | chr9 | 129373287 | 129373573 | Breast Basal + Luminal Epithelium | 0.74 | 0.06 | Intergenic | LMX1B | M |
| 6221 | chr11 | 17497362 | 17497547 | Breast Basal + Luminal Epithelium | 0.78 | 0.12 | intron | ABCC8 | M |
| 6222 | chr6 | 50818692 | 50819121 | Breast Basal + Luminal Epithelium | 0.74 | 0.09 | Intergenic | TFAP2B | M |
| 6223 | chr2 | 220378996 | 220379372 | Breast Basal + Luminal Epithelium | 0.68 | 0.04 | exor | ASIC4 | M |
| 6224 | chr18 | 56939245 | 56939552 | Breast Basal + Luminal Epithelium | 0.73 | 0.1 | intron, exon | RAX, RAX | M |
| 6225 | chr3 | 16554239 | 16554620 | Breast Basal + Luminal Epithelium | 0.64 | 0.03 | intron | RFTN1 | M |
| 6226 | chr1 | 215255164 | 215255506 | Breast Basal + Luminal Epithelium | 0.62 | 0.11 | intron | KCNK2 | M |
| 6227 | chr2 | 232113287 | 232113348 | Lung Alveolar + Bronchial cells | 0.07 | 0.94 | intron | ARMC9 | U |
| 6228 | chr9 | 125983841 | 125984259 | Lung Alveolar + Bronchial cells | 0.05 | 0.91 | intron | STRBP | U |
| 6229 | chr14 | 87899146 | 87899412 | Lung Alveolar + Bronchial cells | 0.09 | 0.91 | Intergenic | LOC283585 | U |
| 6230 | chr1 | 71656617 | 71656873 | Lung Alveolar + Bronchial cells | 0.07 | 0.87 | intron | ZRAN82-AS2 | U |
| 6231 | chr6 | 107532762 | 107532925 | Lung Alveolar + Bronchial cells | 0.15 | 0.94 | intron | POSS2 | U |
| 6232 | chr11 | 75948297 | 75948528 | Lung Alveolar + Bronchial cells | 0.14 | 0.9 | Intergenic | WNT11 | U |
| 6233 | chr4 | 813142 | 813319 | Lung Alveolar + Bronchial cells | 0.09 | 0.82 | intron | CPLX1 | U |
| 6234 | chr5 | 44490688 | 44491054 | Lung Alveolar + Bronchial cells | 0.08 | 0.8 | Intergenic | FGF10 | U |
| 6235 | chr15 | 65519245 | 65519449 | Lung Alveolar + Bronchial cells | 0.1 | 0.82 | Intergenic | CILP | U |
| 6236 | chr3 | 53161967 | 53162111 | Lung Alveolar + Bronchial cells | 0.23 | 0.94 | intron | RFT1 | U |
| 6237 | chr3 | 125617768 | 125618203 | Lung Alveolar + Bronchial cells | 0.21 | 0.89 | Intergenic | FAMB6JP | U |
| 6238 | chr15 | 72470213 | 72470368 | Lung Alveolar + Bronchial cells | 0.23 | 0.9 | intron | GRAMD2 | U |
| 6239 | chr2 | 179044777 | 179045001 | Lung Alveolar + Bronchial cells | 0.26 | 0.9 | Intergenic | RBM45 | U |
| 6240 | chr7 | 66074579 | 66074813 | Lung Alveolar + Bronchial cells | 0.24 | 0.88 | Intergenic | KCTD7 | U |
| 6241 | chr3 | 127724452 | 127724726 | Lung Alveolar + Bronchial cells | 0.16 | 0.8 | Intergenic | SEC61A1 | U |
| 6242 | chr7 | 155189643 | 155189743 | Lung Alveolar + Bronchial cells | 0.28 | 0.86 | Intergenic | EN2 | U |
| 6243 | chr14 | 81469419 | 81469846 | Lung Alveolar + Bronchial cells | 0.33 | 0.89 | intron | TSHR | U |
| 6244 | chr3 | 183485515 | 183485601 | Lung Alveolar + Bronchial cells | 0.03 | 0.9 | intron | YEATS2 | U |
| 6245 | chr8 | 429506 | 429633 | Lung Alveolar + Bronchial cells | 0.05 | 0.89 | Intergenic | C8orf42 | U |
| 6246 | chr2 | 114113839 | 114114244 | Lung Alveolar + Bronchial cells | 0.04 | 0.86 | Intergenic | PAX8 | U |
| 6247 | chr19 | 51593003 | 51593206 | Lung Alveolar + Bronchial cells | 0.09 | 0.9 | Intergenic | KLK14 | U |
| 6248 | chr2 | 230139684 | 230139721 | Lung Alveolar + Bronchial cells | 0.13 | 0.93 | Intergenic | PID1 | U |
| 6249 | chr8 | 48474333 | 48474714 | Lung Alveolar + Bronchial cells | 0.1 | 0.9 | intron | KIAA0146 | U |
| 6250 | chr9 | 948548 | 948854 | Lung Alveolar + Bronchial cells | 0.03 | 0.8 | intron | DMRT1 | U |
| 6251 | chr7 | 50595652 | 50595696 | Lung Alveolar + Bronchial cells | 0.16 | 0.92 | intron | DDC | U |
| 6252 | chr5 | 125859310 | 125859464 | Lung Alveolar + Bronchial cells | 0.14 | 0.9 | Intergenic | ALDH741 | U |
| 6253 | chr2 | 207790148 | 207790370 | Lung Alveolar + Bronchial cells | 0.14 | 0.9 | Intergenic | CPO | U |
| 6254 | chr17 | 19796075 | 19796424 | Lung Alveolar + Bronchial cells | 0.08 | 0.84 | Intergenic | ULK2 | U |
| 6255 | chr1 | 48750621 | 48750695 | Lung Alveolar + Bronchial cells | 0.05 | 0.8 | Intergenic | SLC5A9 | U |
| 6256 | chr6 | 75919914 | 75920126 | Lung Alveolar + Bronchial cells | 0.08 | 0.83 | Intergenic | COL12A1 | U |
| 6257 | chr10 | 129791109 | 129791381 | Lung Alveolar + Bronchial cells | 0.18 | 0.91 | intron | PTPRE | U |
| 6258 | chr3 | 100988813 | 100989001 | Lung Alveolar + Bronchial cells | 0.2 | 0.92 | intron | IMPG2 | U |
| 6259 | chr4 | 3617771 | 3618005 | Lung Alveolar + Bronchial cells | 0.12 | 0.84 | Intergenic | FLI35424 | U |
| 6260 | chr14 | 85435653 | 85435836 | Lung Alveolar + Bronchial cells | 0.12 | 0.83 | Intergenic | FLRT2 | U |
| 6261 | chr6 | 121423085 | 121423311 | Lung Alveolar + Bronchial cells | 0.22 | 0.93 | intron | C6orf170 | U |
| 6262 | chr13 | 106988723 | 106988955 | Lung Alveolar + Bronchial cells | 0.14 | 0.85 | Intergenic | LINC00460 | U |
| 6263 | chr1 | 61509550 | 61509709 | Lung Alveolar + Bronchial cells | 0.13 | 0.83 | Intergenic | NFIA | U |
| 6264 | chr5 | 73367049 | 73367301 | Lung Alveolar + Bronchial cells | 0.2 | 0.9 | Intergenic | ARHGEF28 | U |
| 6265 | chr14 | 95541336 | 95541520 | Lung Alveolar + Bronchial cells | 0.15 | 0.84 | Intergenic | MIR3173 | U |
| 6266 | chr12 | 118298749 | 118298820 | Lung Alveolar + Bronchial cells | 0.24 | 0.92 | intron | KSR2 | U |
| 6267 | chr4 | 4156493 | 4156568 | Lung Alveolar + Bronchial cells | 0.15 | 0.83 | Intergenic | OTOP1 | U |
| 6268 | chr1 | 26214209 | 26214398 | Lung Alveolar + Bronchial cells | 0.2 | 0.88 | intron | STMN1 | U |
| 6269 | chr5 | 180254663 | 180254867 | Lung Alveolar + Bronchial cells | 0.22 | 0.9 | Intergenic | LINC00847 | U |
| 6270 | chr1 | 240077042 | 240077180 | Lung Alveolar + Bronchial cells | 0.23 | 0.89 | Intergenic | RPS7P5 | U |
| 6271 | chr2 | 103454245 | 103454608 | Lung Alveolar + Bronchial cells | 0.15 | 0.81 | Intergenic | TMEM182 | U |
| 6272 | chr11 | 40977506 | 40977873 | Lung Alveolar + Bronchial cells | 0.18 | 0.84 | intron | RRC4C | U |
| 6273 | chr2 | 172855891 | 172856268 | Lung Alveolar + Bronchial cells | 0.2 | 0.86 | Intergenic | METAP1D | U |
| 6274 | chr3 | 27317025 | 27317459 | Lung Alveolar + Bronchial cells | 0.23 | 0.89 | intron | NEK10 | U |
| 6275 | chr16 | 11402006 | 11402119 | Lung Alveolar + Bronchial cells | 0.23 | 0.88 | Intergenic | PRM1 | U |
| 6276 | chr1 | 10560776 | 10560924 | Lung Alveolar + Bronchial cells | 0.24 | 0.89 | intron | PEX14 | U |
| 6277 | chr7 | 71070144 | 71070339 | Lung Alveolar + Bronchial cells | 0.18 | 0.83 | intron | WBSCR17 | U |
| 6278 | chr7 | 3450257 | 3450462 | Lung Alveolar + Bronchial cells | 0.24 | 0.89 | intron | SDK1 | U |
| 6279 | chr21 | 47928925 | 47929342 | Lung Alveolar + Bronchial cells | 0.22 | 0.87 | intron | DIP2A | U |
| 6280 | chr1 | 22762726 | 22762801 | Lung Alveolar + Bronchial cells | 0.24 | 0.88 | Intergenic | ZBTB40 | U |
| 6281 | chr7 | 92042225 | 92042332 | Lung Alveolar + Bronchial cells | 0.14 | 0.78 | Intergenic | GATAD1 | U |
| 6282 | chr1 | 202001636 | 202001794 | Lung Alveolar + Bronchial cells | 0.28 | 0.92 | Intergenic | ELF3 | U |
| 6283 | chr10 | 115875121 | 115875408 | Lung Alveolar + Bronchial cells | 0.2 | 0.84 | Intergenic | MIR2110 | U |
| 6284 | chr2 | 111498715 | 111499057 | Lung Alveolar + Bronchial cells | 0.2 | 0.84 | intron | ACOXL | U |
| 6285 | chr13 | 54890942 | 54891327 | Lung Alveolar + Bronchial cells | 0.22 | 0.86 | Intergenic | MIR1297 | U |
| 6286 | chr1 | 10560188 | 10560258 | Lung Alveolar + Bronchial cells | 0.27 | 0.9 | intron | PEX14 | U |
| 6287 | chr19 | 38548317 | 38548412 | Lung Alveolar + Bronchial cells | 0.2 | 0.82 | intron | SIPA1L3 | U |
| 6288 | chr1 | 46786373 | 46786567 | Lung Alveolar + Bronchial cells | 0.16 | 0.78 | Intergenic | UQCRH | U |
| 6289 | chr16 | 8826332 | 8826534 | Lung Alveolar + Bronchial cells | 0.24 | 0.86 | intron | ABAT | U |
| 6290 | chr15 | 67674267 | 67674507 | Lung Alveolar + Bronchial cells | 0.24 | 0.86 | intron | QCH | U |
| 6291 | chr3 | 127131170 | 127131537 | Lung Alveolar + Bronchial cells | 0.19 | 0.81 | Intergenic | TPRA1 | U |
| 6292 | chr15 | 63244269 | 63244342 | Lung Alveolar + Bronchial cells | 0.29 | 0.9 | Intergenic | TPM1 | U |
| 6293 | chr17 | 76793232 | 76793330 | Lung Alveolar + Bronchial cells | 0.28 | 0.89 | TTS, exon | USP36, USP36 | U |
| 6294 | chr4 | 35107603 | 35107731 | Lung Alveolar + Bronchial cells | 0.27 | 0.88 | Intergenic | ARAP2 | U |
| 6295 | chr18 | 59640487 | 59640653 | Lung Alveolar + Bronchial cells | 0.29 | 0.9 | Intergenic | RNF152 | U |
| 6296 | chr10 | 1136810473 | 113681214 | Lung Alveolar + Bronchial cells | 0.21 | 0.82 | Intergenic | GPAM | U |
| 6297 | chr1 | 99397142 | 99397348 | Lung Alveolar + Bronchial cells | 0.22 | 0.83 | intron | LPPR5 | U |
| 6298 | chr4 | 4263169 | 4263397 | Lung Alveolar + Bronchial cells | 0.23 | 0.84 | Intergenic | TMEM128 | U |
| 6299 | chr5 | 133260671 | 133260929 | Lung Alveolar + Bronchial cells | 0.21 | 0.82 | Intergenic | C5orf15 | U |
| 6300 | chr17 | 15795454 | 15795779 | Lung Alveolar + Bronchial cells | 0.26 | 0.87 | Intergenic | ADORA2B | U |
| 6301 | chr3 | 157003040 | 157003420 | Lung Alveolar + Bronchial cells | 0.22 | 0.83 | intron | VEPH1 | U |
| 6302 | chr12 | 1058728953 | 105873112 | Lung Alveolar + Bronchial cells | 0.22 | 0.82 | Intergenic | C12orf75 | U |
| 6303 | chr10 | 106544867 | 106545102 | Lung Alveolar + Bronchial cells | 0.22 | 0.82 | intron | SORCS3 | U |
| 6304 | chr4 | 8953961 | 8954061 | Lung Alveolar + Bronchial cells | 0.24 | 0.83 | Intergenic | LOC650293 | U |
| 6305 | chr10 | 65836171 | 65836504 | Lung Alveolar + Bronchial cells | 0.28 | 0.87 | Intergenic | REEP3 | U |
| 6306 | chr1 | 65037436 | 65037857 | Lung Alveolar + Bronchial cells | 0.34 | 0.93 | intron | CACHD1 | U |
| 6307 | chr7 | 1569038001 | 156903917 | Lung Alveolar + Bronchial cells | 0.26 | 0.84 | Intergenic | UBE3C | U |
| 6308 | chr19 | 46963970 | 46964187 | Lung Alveolar + Bronchial cells | 0.32 | 0.9 | Intergenic | PNMAL1 | U |
| 6309 | chr4 | 150770095 | 150770431 | Lung Alveolar + Bronchial cells | 0.28 | 0.86 | Intergenic | DCLK2 | U |
| 6310 | chr1 | 18301849 | 18302082 | Lung Alveolar + Bronchial cells | 0.21 | 0.78 | Intergenic | IGSF21 | U |
| 6311 | chr11 | 60166518 | 60166885 | Lung Alveolar + Bronchial cells | 0.28 | 0.85 | intron | MS4A14 | U |
| 6312 | chr5 | 176532975 | 176533169 | Lung Alveolar + Bronchial cells | 0.24 | 0.8 | Intergenic | FGFR4 | U |
| 6313 | chr11 | 1239940043 | 123994267 | Lung Alveolar + Bronchial cells | 0.3 | 0.85 | intron | VWA5A | U |
| 6314 | chr7 | 27546756 | 27547052 | Lung Alveolar + Bronchial cells | 0.31 | 0.86 | Intergenic | HIBADH | U |
| 6315 | chr5 | 158243769 | 158244060 | Lung Alveolar + Bronchial cells | 0.3 | 0.84 | intron | EBF1 | U |
| 6316 | chr1 | 1624360813 | 162436468 | Lung Alveolar + Bronchial cells | 0.32 | 0.86 | Intergenic | UHMK1 | U |
| 6317 | chr3 | 180857211 | 180857631 | Lung Alveolar + Bronchial cells | 0.28 | 0.82 | intron | SOX2-OT | U |
| 6318 | chr7 | 1549610461 | 154961365 | Lung Alveolar + Bronchial cells | 0.26 | 0.79 | Intergenic | LOC100128264 | U |
| 6319 | chr16 | 89361007 | 89361170 | Lung Alveolar + Bronchial cells | 0.34 | 0.86 | intron | ANKRD11 | U |
| 6320 | chr20 | 59700988 | 59701148 | Lung Alveolar + Bronchial cells | 0.34 | 0.85 | Intergenic | CDH4 | U |
| 6321 | chr7 | 1412878 | 1412994 | Lung Alveolar + Bronchial cells | 0.23 | 0.73 | Intergenic | MICALL2 | U |
| 6322 | chr10 | 50374944 | 50375062 | Lung Alveolar + Bronchial cells | 0.29 | 0.79 | exon | C10orf128 | U |
| 6323 | chr19 | 10394627 | 10394792 | Lung Alveolar + Bronchial cells | 0.32 | 0.82 | intron, exon | ICAM1, ICAM1 | U |
| 6324 | chr12 | 48107264 | 48107498 | Lung Alveolar + Bronchial cells | 0.29 | 0.79 | intron | ENDOU | U |
| 6325 | chr11 | 46918967 | 46919454 | Lung Alveolar + Bronchial cells | 0.34 | 0.84 | intron | LRP4 | U |
| 6326 | chr2 | 114344227 | 114344537 | Lung Alveolar + Bronchial cells | 0.33 | 0.78 | intron | WASH2P | U |
| 6327 | chr8 | 111833914 | 111834223 | Lung Alveolar + Bronchial cells | 0.1 | 0.91 | Intergenic | KCNV1 | U |
| 6328 | chr20 | 51000919 | 51001078 | Lung Alveolar + Bronchial cells | 0.08 | 0.85 | Intergenic | ZFP64 | U |
| 6329 | chr3 | 61378641 | 61378874 | Lung Alveolar + Bronchial cells | 0.2 | 0.84 | Intergenic | FHIT | U |
| 6330 | chr14 | 64369053 | 64369469 | Lung Alveolar + Bronchial cells | 0.05 | 0.86 | intron | SYNE2 | U |
| 6331 | chr20 | 60485351 | 60485444 | Lung Alveolar + Bronchial cells | 0.07 | 0.86 | exon, intron | CDH4 | U |
| 6332 | chr9 | 88836212 | 88836389 | Lung Alveolar + Bronchial cells | 0.07 | 0.84 | exon | C9orf153 | U |
| 6333 | chr20 | 23283452 | 23283591 | Lung Alveolar + Bronchial cells | 0.12 | 0.88 | Intergenic | NXT1 | U |
| 6334 | chr1 | 28854250 | 28854589 | Lung Alveolar + Bronchial cells | 0.18 | 0.94 | intron | RCC1 | U |
| 6335 | chr8 | 134602104 | 134602203 | Lung Alveolar + Bronchial cells | 0.16 | 0.91 | Intergenic | ST3GALI | U |
| 6336 | chr9 | 31633242 | 31633446 | Lung Alveolar + Bronchial cells | 0.08 | 0.79 | Intergenic | ACO1 | U |
| 6337 | chr9 | 95771178 | 95771313 | Lung Alveolar + Bronchial cells | 0.12 | 0.92 | intron | FGD3 | U |
| 6338 | chr15 | 91584200 | 91584329 | Lung Alveolar + Bronchial cells | 0.06 | 0.82 | Intergenic | VPS33B | U |
| 6339 | chr22 | 29130426 | 29130713 | Lung Alveolar + Bronchial cells | 0.16 | 0.9 | exon, intron | CHEK2, CHEK2 | U |
| 6340 | chr20 | 60856661 | 60857041 | Lung Alveolar + Bronchial cells | 0.23 | 0.92 | intron | OSBPL2 | U |
| 6341 | chr14 | 72265353 | 72265520 | Lung Alveolar + Bronchial cells | 0.17 | 0.85 | Intergenic | RGS6 | U |
| 6342 | chr21 | 45895657 | 45895854 | Lung Alveolar + Bronchial cells | 0.21 | 0.89 | Intergenic | LRRC3 | U |
| 6343 | chr9 | 78556526 | 78556801 | Lung Alveolar + Bronchial cells | 0.18 | 0.86 | intron | PCSK5 | U |
| 6344 | chr22 | 24210757 | 24210990 | Lung Alveolar + Bronchial cells | 0.22 | 0.87 | exon, intron | SLC2A11, SLC2A11 | U |
| 6345 | chr21 | 37780765 | 37781175 | Lung Alveolar + Bronchial cells | 0.2 | 0.85 | intron | CHAF1B | U |
| 6346 | chr22 | 25317089 | 25317310 | Lung Alveolar + Bronchial cells | 0.24 | 0.87 | intron | SGSM1 | U |
| 6347 | chr3 | 66053848 | 66054080 | Lung Alveolar + Bronchial cells | 0.2 | 0.83 | Intergenic | MAGI1 | U |
| 6348 | chr1 | 1986186 | 1986259 | Lung Alveolar + Bronchial cells | 0.22 | 0.83 | intron | PRKCZ | U |
| 6349 | chr20 | 26272462 | 26272801 | Lung Alveolar + Bronchial cells | 0.27 | 0.87 | Intergenic | LOC284801 | U |
| 6350 | chr22 | 47486098 | 47486245 | Lung Alveolar + Bronchial cells | 0.26 | 0.82 | intron | TBC1D22A | U |
| 6351 | chr22 | 25093141 | 25093496 | Lung Alveolar + Bronchial cells | 0.32 | 0.87 | Intergenic | POM121L108 | U |
| 6352 | chr22 | 19874916 | 19875088 | Lung Alveolar + Bronchial cells | 0.36 | 0.86 | intron | TXNRD2 | U |
| 6353 | chr19 | 10404606 | 10404687 | Lung Alveolar + Bronchial cells | 0.66 | 0.23 | intron | ICAM5 | M |
| 6354 | chr5 | 3602272 | 3602460 | Lung Alveolar + Bronchial cells | 0.82 | 0.15 | TTS | IRX1 | M |
| 6355 | chr5 | 3606284 | 3606496 | Lung Alveolar + Bronchial cells | 0.82 | 0.19 | Intergenic | IRX1 | M |
| 6356 | chr5 | 3602539 | 3602826 | Lung Alveolar + Bronchial cells | 0.71 | 0.15 | Intergenic | IRX1 | M |
| 6357 | chr5 | 3594139 | 3594432 | Lung Alveolar + Bronchial cells | 0.66 | 0.13 | Intergenic | IRX1 | M |
| 6358 | chr5 | 3606587 | 3607009 | Lung Alveolar + Bronchial cells | 0.65 | 0.13 | Intergenic | IRX1 | M |
| 6359 | chr5 | 3600389 | 3600587 | Lung Alveolar + Bronchial cells | 0.7 | 0.2 | intron | IRX1 | M |
| 6360 | chr7 | 113722561 | 113722722 | Lung Alveolar + Bronchial cells | 0.68 | 0.23 | Intergenic | PPP1R3A | M |
| 6361 | chr5 | 43039669 | 43039962 | Lung Alveolar + Bronchial cells | 0.6 | 0.18 | exon | ANXA2R | M |
| 6362 | chr6 | 10416561 | 10416738 | Lung Alveolar + Bronchial cells | 0.66 | 0.25 | TTS | OC100130275 | M |
| 6363 | chr19 | 18715633 | 18715811 | Lung Alveolar + Bronchial cells | 0.63 | 0.26 | intron | CRLF1 | M |
| 6364 | chr19 | 18715870 | 18716183 | Lung Alveolar + Bronchial cells | 0.57 | 0.24 | intron | CRLF1 | M |
| 6365 | chr14 | 61655239 | 61655414 | Lung Alveolar + Bronchial cells | 0.55 | 0.26 | Intergenic | TMEM30B | M |
| 6366 | chr1 | 28726744 | 28726838 | Fallopian + Ovary Epithelium | 0.06 | 0.95 | intron | PHACTR4 | U |
| 6367 | chr4 | 186845917 | 186846165 | Fallopian + Ovary Epithelium | 0.05 | 0.92 | intron | SORBS2 | U |
| 6368 | chr2 | 238519236 | 238519649 | Fallopian + Ovary Epithelium | 0.07 | 0.93 | Intergenic | RAB17 | U |
| 6369 | chr10 | 114144318 | 114144465 | Fallopian + Ovary Epithelium | 0.08 | 0.94 | intron | ACSL5 | U |
| 6370 | chr19 | 36531822 | 36532041 | Fallopian + Ovary Epithelium | 0.07 | 0.92 | intron | THAP8 | U |
| 6371 | chr7 | 150006090 | 150006391 | Fallopian + Ovary Epithelium | 0.07 | 0.92 | intron | ACTR3C | U |
| 6372 | chr13 | 46444682 | 46445045 | Fallopian + Ovary Epithelium | 0.07 | 0.92 | Intergenic | SIAH3 | U |
| 6373 | chr10 | 29819520 | 29819649 | Fallopian + Ovary Epithelium | 0.03 | 0.88 | exon | SVIL | U |
| 6374 | chr2 | 11652968 | 11653250 | Fallopian + Ovary Epithelium | 0.05 | 0.9 | Intergenic | GREB1 | U |
| 6375 | chr4 | 1913187 | 1913522 | Fallopian + Ovary Epithelium | 0.03 | 0.88 | intron | WHSC1 | U |
| 6376 | chr7 | 148907974 | 148908417 | Fallopian + Ovary Epithelium | 0.04 | 0.89 | intron | ZNF282 | U |
| 6377 | chr9 | 131955855 | 131955975 | Fallopian + Ovary Epithelium | 0.04 | 0.88 | Intergenic | ER5L | U |
| 6378 | chr11 | 65438546 | 65438787 | Fallopian + Ovary Epithelium | 0.09 | 0.92 | Intergenic | RELA | U |
| 6379 | chr2 | 227885834 | 227885961 | Fallopian + Ovary Epithelium | 0.12 | 0.92 | intron | COL4A4 | U |
| 6380 | chr17 | 41407341 | 41407473 | Fallopian + Ovary Epithelium | 0.1 | 0.9 | Intergenic | LINC00854 | U |
| 6381 | chr22 | 47227516 | 47227745 | Fallopian + Ovary Epithelium | 0.11 | 0.9 | intron | TBC1D22A | U |
| 6382 | chr2 | 239473028 | 239473083 | Fallopian + Ovary Epithelium | 0.08 | 0.86 | Intergenic | LOC151171 | U |
| 6383 | chr16 | 55418550 | 55418663 | Fallopian + Ovary Epithelium | 0.08 | 0.86 | Intergenic | IRX6 | U |
| 6384 | chr7 | 54901102 | 54901214 | Fallopian + Ovary Epithelium | 0.12 | 0.89 | Intergenic | SEC61G | U |
| 6385 | chr1 | 168194169 | 168194524 | Fallopian + Ovary Epithelium | 0.13 | 0.9 | promoter-TSS | SFT2D2 | U |
| 6386 | chr4 | 47608721 | 4761052 | Fallopian + Ovary Epithelium | 0.17 | 0.92 | Intergenic | MSX1 | U |
| 6387 | chr16 | 89769968 | 89770108 | Fallopian + Ovary Epithelium | 0.19 | 0.92 | Intergenic | SPATA2L | U |
| 6388 | chr4 | 3948578 | 3948902 | Fallopian + Ovary Epithelium | 0.19 | 0.92 | exon, intron | FAM86EP | U |
| 6389 | chr16 | 56828732 | 56828826 | Fallopian + Ovary Epithelium | 0.16 | 0.88 | intron | NUP93 | U |
| 6390 | chr18 | 71680167 | 71680348 | Fallopian + Ovary Epithelium | 0.13 | 0.85 | Intergenic | FBXO15 | U |
| 6391 | chr4 | 837076703 | 83707932 | Fallopian + Ovary Epithelium | 0.19 | 0.91 | intron | SCD5 | U |
| 6392 | chr5 | 16981676 | 16981794 | Fallopian + Ovary Epithelium | 0.24 | 0.91 | Intergenic | MYO10 | U |
| 6393 | chr10 | 3023582 | 3023764 | Fallopian + Ovary Epithelium | 0.18 | 0.85 | Intergenic | PFKP | U |
| 6394 | chr3 | 125643874 | 125643978 | Fallopian + Ovary Epithelium | 0.24 | 0.88 | exon | FAM86JP | U |
| 6395 | chr8 | 41555375 | 41555478 | Fallopian + Ovary Epithelium | 0.25 | 0.88 | intron | ANK1 | U |
| 6396 | chr14 | 93942499 | 93942654 | Fallopian + Ovary Epithelium | 0.21 | 0.84 | intron | UNC79 | U |
| 6397 | chr18 | 3363758 | 3363955 | Fallopian + Ovary Epithelium | 0.26 | 0.89 | Intergenic | TGIF1 | U |
| 6398 | chr22 | 50015331 | 50015414 | Fallopian + Ovary Epithelium | 0.26 | 0.86 | intron | C22orf34 | U |
| 6399 | chr10 | 49794755 | 49795053 | Fallopian + Ovary Epithelium | 0.3 | 0.86 | intron | ARHGAP22 | U |
| 6400 | chr3 | 54879942 | 54880015 | Fallopian + Ovary Epithelium | 0.04 | 0.89 | intron | CACNA2D3 | U |
| 6401 | chr1 | 60271893 | 60271987 | Fallopian + Ovary Epithelium | 0.06 | 0.91 | Intergenic | HOOK1 | U |
| 6402 | chr1 | 28727151 | 28727248 | Fallopian + Ovary Epithelium | 0.11 | 0.96 | intron | PHACTR4 | U |
| 6403 | chr1 | 28727848 | 28727979 | Fallopian + Ovary Epithelium | 0.06 | 0.91 | intron | PHACTR4 | U |
| 6404 | chr17 | 67420643 | 67420803 | Fallopian + Ovary Epithelium | 0.08 | 0.93 | intron | MAP2K6 | U |
| 6405 | chr7 | 120527575 | 120527758 | Fallopian + Ovary Epithelium | 0.04 | 0.88 | Intergenic | TSPAN12 | U |
| 6406 | chr2 | 73987714 | 73988039 | Fallopian + Ovary Epithelium | 0.08 | 0.92 | Intergenic | DUSP11 | U |
| 6407 | chr12 | 97092957 | 97093119 | Fallopian + Ovary Epithelium | 0.05 | 0.88 | Intergenic | NEDD1 | U |
| 6408 | chr19 | 14369505 | 14369811 | Fallopian + Ovary Epithelium | 0.04 | 0.87 | Intergenic | LPHN1 | U |
| 6409 | chr2 | 237233447 | 237233837 | Fallopian + Ovary Epithelium | 0.03 | 0.86 | intron | IQCA1 | U |
| 6410 | chr14 | 104024102 | 104024245 | Fallopian + Ovary Epithelium | 0.08 | 0.9 | exon | BAG5 | U |
| 6411 | chr5 | 88219993 | 88220272 | Fallopian + Ovary Epithelium | 0.05 | 0.87 | Intergenic | MEF2C | U |
| 6412 | chr1 | 64949730 | 64950193 | Fallopian + Ovary Epithelium | 0.09 | 0.91 | intron | CACHD1 | U |
| 6413 | chr17 | 8453016 | 8453251 | Fallopian + Ovary Epithelium | 0.13 | 0.94 | intron | MYH10 | U |
| 6414 | chr4 | 153664211 | 153664573 | Fallopian + Ovary Epithelium | 0.11 | 0.92 | Intergenic | TIGD4 | U |
| 6415 | chr4 | 109633949 | 109634363 | Fallopian + Ovary Epithelium | 0.08 | 0.89 | Intergenic | AGXT2L1 | U |
| 6416 | chr1 | 9980646 | 9981111 | Fallopian + Ovary Epithelium | 0.09 | 0.9 | Intergenic | CTNNBIP1 | U |
| 6417 | chr21 | 46081315 | 46081476 | Fallopian + Ovary Epithelium | 0.07 | 0.87 | lintron | TSPEAR | U |
| 6418 | chr6 | 111611322 | 111611724 | Fallopian + Ovary Epithelium | 0.1 | 0.9 | Intergenic | KIAA1919 | U |
| 6419 | chr2 | 8778679 | 8778785 | Fallopian + Ovary Epithelium | 0.1 | 0.89 | Intergenic | ID2 | U |
| 6420 | chr18 | 45840495 | 45840661 | Fallopian + Ovary Epithelium | 0.12 | 0.91 | Intergenic | ZBTB7C | U |
| 6421 | chr19 | 20061790 | 20062023 | Fallopian + Ovary Epithelium | 0.12 | 0.91 | Intergenic | ZNF93 | U |
| 6422 | chr2 | 120469972 | 120470243 | Fallopian + Ovary Epithelium | 0.08 | 0.87 | Intergenic | TMEM177 | U |
| 6423 | chr1 | 155462172 | 155462510 | Fallopian + Ovary Epithelium | 0.12 | 0.91 | intron | ASH1L | U |
| 6424 | chr19 | 13019339 | 13019434 | Fallopian + Ovary Epithelium | 0.09 | 0.87 | intron | SYCE2 | U |
| 6425 | chr2 | 680602 | 680725 | Fallopian + Ovary Epithelium | 0.06 | 0.84 | Intergenic | TMEM18 | U |
| 6426 | chr5 | 137859050 | 137859180 | Fallopian + Ovary Epithelium | 0.12 | 0.9 | intron | ETF1 | U |
| 6427 | chr10 | 61553339 | 61553474 | Fallopian + Ovary Epithelium | 0.16 | 0.94 | intron | CCDC6 | U |
| 6428 | chr16 | 29645054 | 29645430 | Fallopian + Ovary Epithelium | 0.12 | 0.9 | Intergenic | SLC7A5P1 | U |
| 6429 | chr2 | 71479137 | 71479556 | Fallopian + Ovary Epithelium | 0.06 | 0.84 | Intergenic | PAIP28 | U |
| 6430 | chr2 | 233240148 | 233240277 | Fallopian + Ovary Epithelium | 0.04 | 0.81 | Intergenic | ALPP | U |
| 6431 | chr19 | 3581545 | 3581723 | Fallopian + Ovary Epithelium | 0.06 | 0.83 | Intergenic | GIPC3 | U |
| 6432 | chr18 | 76683099 | 76683177 | Fallopian + Ovary Epithelium | 0.12 | 0.88 | Intergenic | SALL3 | U |
| 6433 | chr11 | 67564256 | 67564379 | Fallopian + Ovary Epithelium | 0.08 | 0.84 | intron | FAM86C2P | U |
| 6434 | chr6 | 168624884 | 168625205 | Fallopian + Ovary Epithelium | 0.13 | 0.89 | Intergenic | DACT2 | U |
| 6435 | chr19 | 49643282 | 49643669 | Fallopian + Ovary Epithelium | 0.06 | 0.82 | exon, intron | PPFIA3, PPFIA3 | U |
| 6436 | chr19 | 11264642 | 11264745 | Fallopian + Ovary Epithelium | 0.14 | 0.89 | intron | SPC24 | U |
| 6437 | chr2 | 206701896 | 206702101 | Fallopian + Ovary Epithelium | 0.18 | 0.93 | Intergenic | NRP2 | U |
| 6438 | chr11 | 68130480 | 68130721 | Fallopian + Ovary Epithelium | 0.05 | 0.8 | intron | LRP5 | U |
| 6439 | chr7 | 106245661 | 106245923 | Fallopian + Ovary Epithelium | 0.16 | 0.91 | Intergenic | CCDC71L | U |
| 6440 | chr7 | 43245855 | 43246154 | Fallopian + Ovary Epithelium | 0.08 | 0.83 | intron | HECW1 | U |
| 6441 | chr1 | 36059437 | 36059775 | Fallopian + Ovary Epithelium | 0.1 | 0.85 | intron | TFAP2E | U |
| 6442 | chr3 | 39620969 | 39621383 | Fallopian + Ovary Epithelium | 0.12 | 0.87 | Intergenic | MOBP | U |
| 6443 | chr1 | 175777363 | 175777412 | Fallopian + Ovary Epithelium | 0.16 | 0.9 | Intergenic | TNR | U |
| 6444 | chr12 | 13149855 | 131498748 | Fallopian + Ovary Epithelium | 0.12 | 0.86 | intron | GPR133 | U |
| 6445 | chr8 | 60160268 | 60160473 | Fallopian + Ovary Epithelium | 0.04 | 0.78 | Intergenic | TOX | U |
| 6446 | chr13 | 96202235 | 96202449 | Fallopian + Ovary Epithelium | 0.15 | 0.89 | intron | CLDN10 | U |
| 6447 | chr3 | 184824922 | 184825195 | Fallopian + Ovary Epithelium | 0.16 | 0.88 | intron | C3orf70 | U |
| 6448 | chr1 | 18916289 | 18916329 | Fallopian + Ovary Epithelium | 0.18 | 0.88 | Intergenic | PAX7 | U |
| 6449 | chr16 | 84066952 | 84067047 | Fallopian + Ovary Epithelium | 0.12 | 0.82 | exon | SLC38A8 | U |
| 6450 | chr11 | 71506971 | 71507101 | Fallopian + Ovary Epithelium | 0.2 | 0.89 | TTS, exon | ALG1L9P, FAM86C1 | U |
| 6451 | chr6 | 37537563 | 37537707 | Fallopian + Ovary Epithelium | 0.17 | 0.86 | Intergenic | MIR4462 | U |
| 6452 | chr16 | 88194681 | 88194754 | Fallopian + Ovary Epithelium | 0.2 | 0.88 | Intergenic | BANP | U |
| 6453 | chr11 | 61264069 | 61264257 | Fallopian + Ovary Epithelium | 0.21 | 0.89 | Intergenic | MIR4488 | U |
| 6454 | chr5 | 138910053 | 138910392 | Fallopian + Ovary Epithelium | 0.26 | 0.94 | Intergenic | UBE2D2 | U |
| 6455 | chr19 | 46302021 | 46302084 | Fallopian + Ovary Epithelium | 0.19 | 0.86 | intron | RSPH6A | U |
| 6456 | chr21 | 47256526 | 47256617 | Fallopian + Ovary Epithelium | 0.22 | 0.89 | promoter-TSS | LOC100129027 | U |
| 6457 | chr9 | 138912737 | 138912911 | Fallopian + Ovary Epithelium | 0.24 | 0.91 | intron | NACC2 | U |
| 6458 | chr9 | 1403229253 | 140323153 | Fallopian + Ovary Epithelium | 0.1 | 0.77 | intron | NOXA1 | U |
| 6459 | chr5 | 138902796 | 138903153 | Fallopian + Ovary Epithelium | 0.22 | 0.89 | Intergenic | UBE2D2 | U |
| 6460 | chr11 | 36501472 | 36501671 | Fallopian + Ovary Epithelium | 0.18 | 0.83 | Intergenic | TRAF6 | U |
| 6461 | chr9 | 135749839 | 135750061 | Fallopian + Ovary Epithelium | 0.26 | 0.91 | intron | AK8 | U |
| 6462 | chr2 | 55498546 | 55498796 | Fallopian + Ovary Epithelium | 0.28 | 0.93 | Intergenic | MTIF2 | U |
| 6463 | chr3 | 710009411 | 71001302 | Fallopian + Ovary Epithelium | 0.19 | 0.84 | Intergenic | MIR1284 | U |
| 6464 | chr8 | 94685763 | 94686193 | Fallopian + Ovary Epithelium | 0.24 | 0.89 | intron | LINC00535 | U |
| 6465 | chrX | 43923373 | 43923659 | Fallopian + Ovary Epithelium | 0.11 | 0.75 | Intergenic | NDP | U |
| 6466 | chr11 | 68130181 | 68130435 | Fallopian + Ovary Epithelium | 0.25 | 0.88 | intron | LRP5 | U |
| 6467 | chr11 | 73654734 | 73655118 | Fallopian + Ovary Epithelium | 0.26 | 0.89 | Intergenic | DNAJB13 | U |
| 6468 | chr1 | 213240300 | 213240636 | Fallopian + Ovary Epithelium | 0.32 | 0.91 | intron | RPS6KC1 | U |
| 6469 | chr4 | 19457447 | 19457733 | Fallopian + Ovary Epithelium | 0.1 | 0.91 | Intergenic | SLIT2 | U |
| 6470 | chr5 | 152127502 | 152127822 | Fallopian + Ovary Epithelium | 0.04 | 0.82 | promoter-TSS | ESR1 | U |
| 6471 | chr21 | 33202023 | 33202411 | Fallopian + Ovary Epithelium | 0.03 | 0.81 | Intergenic | HUNK | U |
| 6472 | chr4 | 19457853 | 19458286 | Fallopian + Ovary Epithelium | 0.08 | 0.86 | Intergenic | SLIT2 | U |
| 6473 | chr6 | 41434943 | 41435059 | Fallopian + Ovary Epithelium | 0.05 | 0.82 | Intergenic | FOXP4 | U |
| 6474 | chr8 | 55222361 | 55222748 | Fallopian + Ovary Epithelium | 0.18 | 0.82 | Intergenic | SOX17 | U |
| 6475 | chr1 | 31506114 | 31506570 | Fallopian + Ovary Epithelium | 0.32 | 0.94 | intron | PUM1 | U |
| 6476 | chr14 | 105688093 | 105688234 | Fallopian + Ovary Epithelium | 0.05 | 0.93 | exon | BRF1 | U |
| 6477 | chr22 | 41092705 | 41092855 | Fallopian + Ovary Epithelium | 0.05 | 0.93 | Intergenic | MCHR1 | U |
| 6478 | chr8 | 101891130 | 101891242 | Fallopian + Ovary Epithelium | 0.07 | 0.95 | Intergenic | YWHAZ | U |
| 6479 | chr20 | 23427139 | 23427226 | Fallopian + Ovary Epithelium | 0.05 | 0.91 | Intergenic | CST11 | U |
| 6480 | chr17 | 59161841 | 59162243 | Fallopian + Ovary Epithelium | 0.1 | 0.95 | intron | BCAS3 | U |
| 6481 | chr13 | 1137976543 | 113797982 | Fallopian + Ovary Epithelium | 0.08 | 0.92 | intron | F10 | U |
| 6482 | chr10 | 11405274 | 11405488 | Fallopian + Ovary Epithelium | 0.06 | 0.89 | Intergenic | USPSNI | U |
| 6483 | chr19 | 30324602 | 30324850 | Fallopian + Ovary Epithelium | 0.09 | 0.88 | Intergenic | CONE1 | U |
| 6484 | chr6 | 37033614 | 37034071 | Fallopian + Ovary Epithelium | 0.14 | 0.91 | Intergenic | FGD2 | U |
| 6485 | chr10 | 131647377 | 131647543 | Fallopian + Ovary Epithelium | 0.13 | 0.89 | intron | EBF3 | U |
| 6486 | chr7 | 148901541 | 148901717 | Fallopian + Ovary Epithelium | 0.15 | 0.9 | intron | ZNF282 | U |
| 6487 | chr10 | 130652036 | 130652285 | Fallopian + Ovary Epithelium | 0.11 | 0.86 | Intergenic | MGMT | U |
| 6488 | chr10 | 131647595 | 131647853 | Fallopian + Ovary Epithelium | 0.15 | 0.89 | intron | EBF3 | U |
| 6489 | chr9 | 132633613 | 132633908 | Fallopian + Ovary Epithelium | 0.23 | 0.95 | intron | USP20 | U |
| 6490 | chr9 | 90086305 | 90086558 | Fallopian + Ovary Epithelium | 0.27 | 0.91 | Intergenic | DAPK1 | U |
| 6491 | chr22 | 29155544 | 29155729 | Fallopian + Ovary Epithelium | 0.2 | 0.83 | Intergenic, TTS | CCDC117, HSCB | U |
| 6492 | chr18 | 74419723 | 74419877 | Fallopian + Ovary Epithelium | 0.02 | 0.88 | Intergenic | LOC100131655 | U |
| 6493 | chr1 | 46963395 | 469634851 | Fallopian + Ovary Epithelium | 0.09 | 0.94 | Intergenic | DMBX1 | U |
| 6494 | chr1 | 54009680 | 54010061 | Fallopian + Ovary Epithelium | 0.03 | 0.87 | intron | GLIS1 | U |
| 6495 | chr1 | 61913481 | 61913963 | Fallopian + Ovary Epithelium | 0.05 | 0.89 | intron | NFIA | U |
| 6496 | chr22 | 47441209 | 47441515 | Fallopian + Ovary Epithelium | 0.06 | 0.89 | intron | TBC1D22A | U |
| 6497 | chr15 | 101464434 | 101464934 | Fallopian + Ovary Epithelium | 0.06 | 0.89 | intron, exon | LRRK1, LRRK1 | U |
| 6498 | chr6 | 152125965 | 152126107 | Fallopian + Ovary Epithelium | 0.02 | 0.84 | promoter-TSS | ESR1 | U |
| 6499 | chr14 | 105688243 | 105688658 | Fallopian + Ovary Epithelium | 0.03 | 0.85 | intron | BRF1 | U |
| 6500 | chr19 | 36531417 | 36531743 | Fallopian + Ovary Epithelium | 0.09 | 0.9 | intron | THAP8 | U |
| 6501 | chr18 | 74167957 | 74168295 | Fallopian + Ovary Epithelium | 0.08 | 0.87 | intron | ZNF516 | U |
| 6502 | chr11 | 1123706373 | 112370778 | Fallopian + Ovary Epithelium | 0.12 | 0.89 | Intergenic | C11orf34 | U |
| 6503 | chr6 | 2867896 | 2868364 | Fallopian + Ovary Epithelium | 0.06 | 0.83 | intron | MGC39372 | U |
| 6504 | chr20 | 395421223 | 39542312 | Fallopian + Ovary Epithelium | 0.09 | 0.84 | Intergenic | TOP1 | U |
| 6505 | chr9 | 90928169 | 90928398 | Fallopian + Ovary Epithelium | 0.06 | 0.81 | Intergenic | SPIN1 | U |
| 6506 | chr14 | 30383284 | 30383478 | Fallopian + Ovary Epithelium | 0.18 | 0.92 | intron | PRKD1 | U |
| 6507 | chr22 | 29586131 | 29586424 | Fallopian + Ovary Epithelium | 0.16 | 0.89 | Intergenic | EMID1 | U |
| 6508 | chr14 | 75363159 | 75363611 | Fallopian + Ovary Epithelium | 0.2 | 0.93 | intron | DLST | U |
| 6509 | chr22 | 39432912 | 39433066 | Fallopian + Ovary Epithelium | 0.2 | 0.9 | Intergenic | APOBEC3F | U |
| 6510 | chr12 | 131615178 | 131615311 | Fallopian + Ovary Epithelium | 0.22 | 0.91 | intron | GPR133 | U |
| 6511 | chr9 | 884909513 | 88491143 | Fallopian + Ovary Epithelium | 0.24 | 0.93 | Intergenic | NAA35 | U |
| 6512 | chr10 | 80232442 | 80232854 | Fallopian + Ovary Epithelium | 0.18 | 0.84 | Intergenic | LINC00595 | U |
| 6513 | chr19 | 418507 | 418743 | Fallopian + Ovary Epithelium | 0.26 | 0.9 | intron | SHC2 | U |
| 6514 | chr20 | 36990773 | 36990862 | Fallopian + Ovary Epithelium | 0.3 | 0.92 | intron | LBP | U |
| 6515 | chr21 | 33897933 | 33898194 | Fallopian + Ovary Epithelium | 0.34 | 0.9 | Intergenic | TCP10L | U |
| 6516 | chr1 | 42357334 | 42357610 | Fallopian + Ovary Epithelium | 0.84 | 0.14 | intron | HIVEP3 | M |
| 6517 | chr2 | 233246379 | 233246595 | Fallopian + Ovary Epithelium | 0.76 | 0.1 | exon | ALPP | M |
| 6518 | chr10 | 22725538 | 22725642 | Fallopian + Ovary Epithelium | 0.77 | 0.22 | exon | LOC100499489 | M |
| 6519 | chr5 | 148521594 | 148521741 | Fallopian + Ovary Epithelium | 0.7 | 0.18 | exon | ABLIM3 | M |
| 6520 | chr2 | 164205032 | 164205324 | Fallopian + Ovary Epithelium | 0.67 | 0.15 | Intergenic | FIGN | M |
| 6521 | chr5 | 124071085 | 124071125 | Fallopian + Ovary Epithelium | 0.62 | 0.15 | intron | ZNF60B | M |
| 6522 | chr5 | 87066677 | 87066817 | Fallopian + Ovary Epithelium | 0.64 | 0.2 | Intergenic | CCNH | M |
| 6523 | chr10 | 62761559 | 62761642 | Fallopian + Ovary Epithelium | 0.62 | 0.26 | promoter-TSS | RHOBTB1 | M |
| 6524 | chr19 | 49112138 | 49112279 | Fallopian + Ovary Epithelium | 0.62 | 0.27 | intron | FAM83E | M |
| 6525 | chrX | 129306199 | 129306382 | Fallopian + Ovary Epithelium | 0.6 | 0.29 | exon | RAB33A | M |
| 6526 | chr7 | 94023466 | 94023568 | Fallopian + Ovary Epithelium | 0.51 | 0.22 | promoter-TSS | COL1A2 | M |
| 6527 | chr8 | 16801894 | 16801959 | Fallopian + Ovary Epithelium | 0.54 | 0.27 | Intergenic | FGF20 | M |
| 6528 | chr5 | 92935126 | 92935269 | Fallopian + Ovary Epithelium | 0.87 | 0.16 | intron | MIR548AO | M |
| 6529 | chr5 | 92938149 | 92938233 | Fallopian + Ovary Epithelium | 0.85 | 0.2 | intron | MIR548AO | M |
| 6530 | chr2 | 2332843191 | 233284634 | Fallopian + Ovary Epithelium | 0.74 | 0.11 | Intergenic | ALPPL2 | M |
| 6531 | chr5 | 92906434 | 42906508 | Fallopian + Ovary Epithelium | 0.82 | 0.23 | promoter-TSS | FLI42709 | M |
| 6532 | chr5 | 138923135 | 138923219 | Fallopian + Ovary Epithelium | 0.68 | 0.09 | Intergenic | UBE2D2 | M |
| 6533 | chr2 | 233284129 | 233284309 | Fallopian + Ovary Epithelium | 0.66 | 0.08 | Intergenic | ALPPL2 | M |
| 6534 | chr2 | 54798129 | 54798348 | Fallopian + Ovary Epithelium | 0.64 | 0.08 | intron | SPTBN1 | M |
| 6535 | chr2 | 233284661 | 233284790 | Fallopian + Ovary Epithelium | 0.7 | 0.16 | Intergenic | ALPPL2 | M |
| 6536 | chr12 | 85671785 | 85671911 | Fallopian + Ovary Epithelium | 0.7 | 0.18 | Intergenic | ALX1 | M |
| 6537 | chr7 | 28995391 | 28995418 | Fallopian + Ovary Epithelium | 0.73 | 0.24 | exon | TRIL | M |
| 6538 | chr12 | 34261382 | 34261570 | Fallopian + Ovary Epithelium | 0.66 | 0.23 | Intergenic | ALG10 | M |
| 6539 | chr3 | 151985697 | 151985919 | Fallopian + Ovary Epithelium | 0.58 | 0.15 | promoter-TSS | MBNL1 | M |
| 6540 | chrX | 21676306 | 21676404 | Fallopian + Ovary Epithelium | 0.64 | 0.27 | promoter-TSS, exon | KLHL34, KLHL34 | M |
| 6541 | chr17 | 10704453 | 10704606 | Gastric + Small Intes. + Colon Epithelium | 0.03 | 0.93 | intron | LINC00675 | U |
| 6542 | chr3 | 18664732 | 18665034 | Gastric + Small Intes. + Colon Epithelium | 0.02 | 0.89 | Intergenic | SATB1 | U |
| 6543 | chr5 | 98112561 | 98112785 | Gastric + Small Intes. + Colon Epithelium | 0.06 | 0.92 | intron | RGMB | U |
| 6544 | chr19 | 19691202 | 19691615 | Gastric + Small Intes. + Colon Epithelium | 0.04 | 0.9 | intron | PBX4 | U |
| 6545 | chr8 | 128582449 | 128582939 | Gastric + Small Intes. + Colon Epithelium | 0.02 | 0.88 | Intergenic | LOC727677 | U |
| 6546 | chr19 | 50680078 | 50680329 | Gastric + Small Intes. + Colon Epithelium | 0.06 | 0.91 | Intergenic | IZUMO2 | U |
| 6547 | chr12 | 33051893 | 33052278 | Gastric + Small Intes. + Colon Epithelium | 0.03 | 0.88 | Intergenic | PKP2 | U |
| 6548 | chr3 | 23550224 | 23550488 | Gastric + Small Intes. + Colon Epithelium | 0.08 | 0.92 | intron | UBE2E2 | U |
| 6549 | chr11 | 4129491 | 4129655 | Gastric + Small Intes. + Colon Epithelium | 0.12 | 0.96 | intron | RRM1 | U |
| 6550 | chr18 | 2639105 | 2639190 | Gastric + Small Intes. + Colon Epithelium | 0.07 | 0.89 | Intergenic | CBX3P2 | U |
| 6551 | chr2 | 58008344 | 58008717 | Gastric + Small Intes. + Colon Epithelium | 0.11 | 0.91 | Intergenic | VRK2 | U |
| 6552 | chr8 | 8733962 | 8734060 | Gastric + Small Intes. + Colon Epithelium | 0.14 | 0.94 | intron | MFHAS1 | U |
| 6553 | chr2 | 1319261423 | 131926444 | Gastric + Small Intes. + Colon Epithelium | 0.05 | 0.84 | Intergenic | POTEE | U |
| 6554 | chr12 | 124723205 | 124723337 | Gastric + Small Intes. + Colon Epithelium | 0.12 | 0.87 | intron | ZNF664-FAM101A | U |
| 6555 | chr2 | 233459533 | 233459843 | Gastric + Small Intes. + Colon Epithelium | 0.15 | 0.88 | Intergenic | EFHD1 | U |
| 6556 | chr1 | 23028593 | 23028919 | Gastric + Small Intes. + Colon Epithelium | 0.03 | 0.75 | Intergenic | EPHB2 | U |
| 6557 | chr4 | 38540553 | 38540632 | Gastric + Small Intes. + Colon Epithelium | 0.07 | 0.96 | Intergenic | KLF3 | U |
| 6558 | chr14 | 78141056 | 78141550 | Gastric + Small Intes. + Colon Epithelium | 0.02 | 0.93 | intron | ALKBH1 | U |
| 6559 | chr13 | 30707879 | 30707974 | Gastric + Small Intes. + Colon Epithelium | 0.01 | 0.91 | Intergenic | KATNAL1 | U |
| 6560 | chr17 | 70409454 | 70409817 | Gastric + Small Intes. + Colon Epithelium | 0.02 | 0.91 | intron | LINC00673 | U |
| 6561 | chr13 | 30707459 | 30707772 | Gastric + Small Intes. + Colon Epithelium | 0.05 | 0.94 | Intergenic | KATNAL1 | U |
| 6562 | chr3 | 71580226 | 71580368 | Gastric + Small Intes. + Colon Epithelium | 0.02 | 0.88 | intron | FOXP1 | U |
| 6563 | chr12 | 124723891 | 124724056 | Gastric + Small Intes. + Colon Epithelium | 0.09 | 0.93 | intron | ZNF664-FAM101A | U |
| 6564 | chr17 | 15855461 | 15855854 | Gastric + Small Intes. + Colon Epithelium | 0.06 | 0.85 | intron | ADORA2B | U |
| 6565 | chr1 | 16525041 | 1652792 | Gastric + Small Intes. + Colon Epithelium | 0.14 | 0.92 | intron | CDK11A | U |
| 6566 | chr2 | 174111136 | 174111588 | Gastric + Small Intes. + Colon Epithelium | 0.02 | 0.91 | intron | ZAK | U |
| 6567 | chr8 | 126371226 | 126371457 | Gastric + Small Intes. + Colon Epithelium | 0.08 | 0.96 | intron | NSMCE2 | U |
| 6568 | chr12 | 117400776 | 117401265 | Gastric + Small Intes. + Colon Epithelium | 0.06 | 0.93 | intron | FBXW8 | U |
| 6569 | chr15 | 59860677 | 59860817 | Gastric + Small Intes. + Colon Epithelium | 0.04 | 0.89 | Intergenic | GCNT3 | U |
| 6570 | chr12 | 98968977 | 98969435 | Gastric + Small Intes. + Colon Epithelium | 0.05 | 0.88 | Intergenic | SLC25A3 | U |
| 6571 | chr16 | 31034362 | 31034406 | Gastric + Small Intes. + Colon Epithelium | 0.05 | 0.87 | Intergenic | STX4 | U |
| 6572 | chr11 | 670910571 | 67091248 | Gastric + Small Intes. + Colon Epithelium | 0.08 | 0.89 | intron | LOC100130987 | U |
| 6573 | chr7 | 103033055 | 103033110 | Gastric + Small Intes. + Colon Epithelium | 0.1 | 0.89 | Intron | SLC26A5 | U |
| 6574 | chr6 | 132596447 | 132596560 | Gastric + Small Intes. + Colon Epithelium | 0.01 | 0.8 | Intergenic | MOXD1 | U |
| 6575 | chr12 | 120131739 | 120131941 | Gastric + Small Intes. + Colon Epithelium | 0.06 | 0.84 | intron | CIT | U |
| 6576 | chr7 | 106659628 | 106659758 | Gastric + Small Intes. + Colon Epithelium | 0.05 | 0.82 | Intergenic | PRKAR2B | U |
| 6577 | chr10 | 123809806 | 123809984 | Gastric + Small Intes. + Colon Epithelium | 0.13 | 0.89 | exon, intron | TACC2 | U |
| 6578 | chr6 | 126312588 | 126312738 | Gastric + Small Intes. + Colon Epithelium | 0.13 | 0.88 | intron | TRMT11 | U |
| 6579 | chr5 | 1165489 | 1165743 | Gastric + Small Intes. + Colon Epithelium | 0.07 | 0.8 | Intergenic | SLC6A19 | U |
| 6580 | chr16 | 29263424 | 29263670 | Gastric + Small Intes. + Colon Epithelium | 0.19 | 0.91 | Intergenic | SNX29P2 | U |
| 6581 | chr8 | 41673482 | 41673863 | Gastric + Small Intes. + Colon Epithelium | 0.21 | 0.91 | intron | ANK1 | U |
| 6582 | chr12 | 76342313 | 76342367 | Gastric + Small Intes. + Colon Epithelium | 0.25 | 0.93 | Intergenic | PHLDA1 | U |
| 6583 | chr5 | 55076926 | 55077411 | Gastric + Small Intes. + Colon Epithelium | 0.21 | 0.88 | intron | DDX4 | U |
| 6584 | chr3 | 175546376 | 175546628 | Gastric + Small Intes. + Colon Epithelium | 0.18 | 0.82 | Intergenic | MIR4789 | U |
| 6585 | chr6 | 141181297 | 141181343 | Gastric + Small Intes. + Colon Epithelium | 0.27 | 0.9 | Intergenic | MIR4465 | U |
| 6586 | chr1 | 28803742 | 28803838 | Gastric + Small Intes. + Colon Epithelium | 0.31 | 0.94 | intron | PHACTR4 | U |
| 6587 | chr17 | 7178011 | 7178055 | Gastric + Small Intes. + Colon Epithelium | 0.25 | 0.87 | Intergenic | SLC2A4 | U |
| 6588 | chr18 | 54641748 | 54642012 | Gastric + Small Intes. + Colon Epithelium | 0.32 | 0.9 | intron | WDR7 | U |
| 6589 | chr2 | 159719993 | 159720281 | Gastric + Small Intes. + Colon Epithelium | 0.33 | 0.9 | Intergenic | DAPL1 | U |
| 6590 | chr13 | 24720631 | 24720999 | Gastric + Small Intes. + Colon Epithelium | 0.04 | 0.88 | Intergenic | SPATA13 | U |
| 6591 | chr12 | 52740967 | 52741076 | Gastric + Small Intes. + Colon Epithelium | 0.02 | 0.84 | Intergenic | KRT85 | U |
| 6592 | chr7 | 100877035 | 100877313 | Gastric + Small Intes. + Colon Epithelium | 0.03 | 0.85 | intron | CLDN15 | U |
| 6593 | chr5 | 148983296 | 148983454 | Gastric + Small Intes. + Colon Epithelium | 0.08 | 0.89 | intron | ARHGEF37 | U |
| 6594 | chr11 | 129283909 | 129284042 | Gastric + Small Intes. + Colon Epithelium | 0.09 | 0.89 | intron | BARX2 | U |
| 6595 | chr7 | 1395170083 | 139517151 | Gastric + Small Intes. + Colon Epithelium | 0.09 | 0.89 | intron | TEXAS1 | U |
| 6596 | chr1 | 230397581 | 230397743 | Gastric + Small Intes. + Colon Epithelium | 0.08 | 0.88 | intron | GALNT2 | U |
| 6597 | chr6 | 1478792401 | 147879592 | Gastric + Small Intes. + Colon Epithelium | 0.06 | 0.86 | intron | SAMD5 | U |
| 6598 | chr4 | 31125721 | 31126053 | Gastric + Small Intes. + Colon Epithelium | 0.02 | 0.81 | intron | PCDH7 | U |
| 6599 | chr3 | 141660918 | 141661299 | Gastric + Small Intes. + Colon Epithelium | 0.07 | 0.86 | Intergenic | ATP183 | U |
| 6600 | chr4 | 142599879 | 142600049 | Gastric + Small Intes. + Colon Epithelium | 0.09 | 0.87 | intron | IL15 | U |
| 6601 | chr17 | 76988813 | 76989063 | Gastric + Small Intes. + Colon Epithelium | 0.14 | 0.92 | exon | CANT1 | U |
| 6602 | chr7 | 66418219 | 66418377 | Gastric + Small Intes. + Colon Epithelium | 0.12 | 0.87 | exon | TMEM248 | U |
| 6603 | chr10 | 108257664 | 108257878 | Gastric + Small Intes. + Colon Epithelium | 0.09 | 0.84 | Intergenic | SORCS1 | U |
| 6604 | chr6 | 138300458 | 138300682 | Gastric + Small Intes. + Colon Epithelium | 0.16 | 0.91 | Intergenic | LOC100130476 | U |
| 6605 | chr4 | 68625812 | 68626218 | Gastric + Small Intes. + Colon Epithelium | 0.1 | 0.85 | Intergenic | GNRHR | U |
| 6606 | chr17 | 73379651 | 73379752 | Gastric + Small Intes. + Colon Epithelium | 0.17 | 0.91 | intron | GRB2 | U |
| 6607 | chr15 | 51133407 | 51133464 | Gastric + Small Intes. + Colon Epithelium | 0.15 | 0.88 | Intergenic | AP4E1 | U |
| 6608 | chr7 | 123677859 | 123677930 | Gastric + Small Intes. + Colon Epithelium | 0.16 | 0.89 | Intergenic | TMEM229A | U |
| 6609 | chr2 | 74690813 | 74690992 | Gastric + Small Intes. + Colon Epithelium | 0.19 | 0.92 | intron | MOGS | U |
| 6610 | chr12 | 125095292 | 125095366 | Gastric + Small Intes. + Colon Epithelium | 0.08 | 0.79 | Intergenic | NCOR2 | U |
| 6611 | chr8 | 22218191 | 22218378 | Gastric + Small Intes. + Colon Epithelium | 0.14 | 0.85 | Intergenic | SLC39A14 | U |
| 6612 | chr8 | 142199816 | 142200027 | Gastric + Small Intes. + Colon Epithelium | 0.15 | 0.85 | intron | DENND3 | U |
| 6613 | chr21 | 40981788 | 40982005 | Gastric + Small Intes. + Colon Epithelium | 0.16 | 0.86 | intron | C21orf88 | U |
| 6614 | chr5 | 139522630 | 139522751 | Gastric + Small Intes. + Colon Epithelium | 0.12 | 0.81 | Intergenic | IGIP | U |
| 6615 | chr17 | 62118078 | 62118302 | Gastric + Small Intes. + Colon Epithelium | 0.22 | 0.91 | Intergenic | ICAM2 | U |
| 6616 | chr1 | 244865982 | 244866367 | Gastric + Small Intes. + Colon Epithelium | 0.26 | 0.95 | intron | DESI2 | U |
| 6617 | chr2 | 64480705 | 64480842 | Gastric + Small Intes. + Colon Epithelium | 0.15 | 0.83 | Intergenic | LINC00309 | U |
| 6618 | chr11 | 129747565 | 129747747 | Gastric + Small Intes. + Colon Epithelium | 0.2 | 0.88 | intron | NFRKB | U |
| 6619 | chr9 | 1324457363 | 132445781 | Gastric + Small Intes. + Colon Epithelium | 0.24 | 0.91 | intron | PRRX2 | U |
| 6620 | chr11 | 110303295 | 110303473 | Gastric + Small Intes. + Colon Epithelium | 0.26 | 0.92 | intron | FDX1 | U |
| 6621 | chr21 | 42874221 | 42874513 | Gastric + Small Intes. + Colon Epithelium | 0.18 | 0.84 | intron | TMPRSS2 | U |
| 6622 | chr3 | 177003438 | 177003633 | Gastric + Small Intes. + Colon Epithelium | 0.27 | 0.92 | Intergenic | TBL1XR1 | U |
| 6623 | chr12 | 125095370 | 125095615 | Gastric + Small Intes. + Colon Epithelium | 0.19 | 0.84 | Intergenic | INCOR2 | U |
| 6624 | chr11 | 125048827 | 125048888 | Gastric + Small Intes. + Colon Epithelium | 0.24 | 0.88 | intron | PKNOX2 | U |
| 6625 | chr3 | 39196897 | 39196974 | Gastric + Small Intes. + Colon Epithelium | 0.28 | 0.92 | Intergenic | CSRNP1 | U |
| 6626 | chr13 | 114312460 | 114312578 | Gastric + Small Intes. + Colon Epithelium | 0.16 | 0.8 | promoter-TSS | ATP4B | U |
| 6627 | chr7 | 104639370 | 104639559 | Gastric + Small Intes. + Colon Epithelium | 0.24 | 0.88 | Intergenic | LOC100216546 | U |
| 6628 | chr10 | 121489841 | 121489918 | Gastric + Small Intes. + Colon Epithelium | 0.28 | 0.91 | intron | INPP5F | U |
| 6629 | chr8 | 11407620 | 11407735 | Gastric + Small Intes. + Colon Epithelium | 0.17 | 0.8 | exon | BLK | U |
| 6630 | chr9 | 131838134 | 131838274 | Gastric + Small Intes. + Colon Epithelium | 0.25 | 0.88 | Intergenic | DOLPP1 | U |
| 6631 | chr5 | 22095854 | 22096016 | Gastric + Small Intes. + Colon Epithelium | 0.23 | 0.86 | intron | CDH12 | U |
| 6632 | chr2 | 67365757 | 67366230 | Gastric + Small Intes. + Colon Epithelium | 0.26 | 0.89 | intron | LOC644838 | U |
| 6633 | chr13 | 49945486 | 49945601 | Gastric + Small Intes. + Colon Epithelium | 0.28 | 0.89 | intron | CAB39L | U |
| 6634 | chr8 | 140787482 | 140787844 | Gastric + Small Intes. + Colon Epithelium | 0.28 | 0.89 | intron | TRAPPC9 | U |
| 6635 | chr5 | 37703053 | 37703464 | Gastric + Small Intes. + Colon Epithelium | 0.24 | 0.85 | Intergenic | MDGA1 | U |
| 6636 | chr17 | 9851673 | 9851768 | Gastric + Small Intes. + Colon Epithelium | 0.24 | 0.84 | intron | GAS7 | U |
| 6637 | chr17 | 46028254 | 46028421 | Gastric + Small Intes. + Colon Epithelium | 0.3 | 0.9 | Intergenic, TTS | PRR15L, PRR15L | U |
| 6638 | chr2 | 235886360 | 235886533 | Gastric + Small Intes. + Colon Epithelium | 0.29 | 0.89 | intron | SH38P4 | U |
| 6639 | chr1 | 29534755 | 29534960 | Gastric + Small Intes. + Colon Epithelium | 0.3 | 0.9 | intron | MECR | U |
| 6640 | chr14 | 90164823 | 90165193 | Gastric + Small Intes. + Colon Epithelium | 0.27 | 0.87 | Intergenic | FOXN3-AS2 | U |
| 6641 | chr5 | 67613688 | 67614145 | Gastric + Small Intes. + Colon Epithelium | 0.28 | 0.88 | Intergenic | PIK3R1 | U |
| 6642 | chr2 | 40228745 | 40228795 | Gastric + Small Intes. + Colon Epithelium | 0.31 | 0.9 | intron | SLC8A1-AS1 | U |
| 6643 | chr1 | 60955862 | 60956009 | Gastric + Small Intes. + Colon Epithelium | 0.3 | 0.89 | Intergenic | C1orf87 | U |
| 6644 | chr4 | 7211949 | 7212134 | Gastric + Small Intes. + Colon Epithelium | 0.28 | 0.86 | intron | SORCS2 | U |
| 6645 | chr3 | 33098511 | 33098712 | Gastric + Small Intes. + Colon Epithelium | 0.32 | 0.9 | intron | GLB1 | U |
| 6646 | chr8 | 124537847 | 124538054 | Gastric + Small Intes. + Colon Epithelium | 0.29 | 0.87 | intron | FBXO32 | U |
| 6647 | chr5 | 1319823 | 1320209 | Gastric + Small Intes. + Colon Epithelium | 0.31 | 0.89 | intron | CLPTM1L | U |
| 6648 | chr7 | 36119355 | 36119648 | Gastric + Small Intes. + Colon Epithelium | 0.3 | 0.86 | Intergenic | EEPD1 | U |
| 6649 | chr4 | 57163146 | 57163247 | Gastric + Small Intes. + Colon Epithelium | 0.32 | 0.87 | intron | KIAA1211 | U |
| 6650 | chr2 | 165235177 | 165235373 | Gastric + Small Intes. + Colon Epithelium | 0.29 | 0.84 | Intergenic | GRB14 | U |
| 6651 | chr2 | 106226918 | 106227135 | Gastric + Small Intes. + Colon Epithelium | 0.28 | 0.83 | promoter-TSS | LOC285000 | U |
| 6652 | chr1 | 60956073 | 60956144 | Gastric + Small Intes. + Colon Epithelium | 0.28 | 0.82 | Intergenic | C1orf87 | U |
| 6653 | chr8 | 1133848003 | 113384877 | Gastric + Small Intes. + Colon Epithelium | 0.3 | 0.84 | intron | CSMD3 | U |
| 6654 | chr9 | 139469784 | 139469879 | Gastric + Small Intes. + Colon Epithelium | 0.34 | 0.88 | Intergenic | MIR4674 | U |
| 6655 | chr12 | 1109136903 | 110913931 | Gastric + Small Intes. + Colon Epithelium | 0.4 | 0.94 | intron | FAM216A | U |
| 6656 | chr13 | 42004907 | 42005262 | Gastric + Small Intes. + Colon Epithelium | 0.32 | 0.86 | Intergenic | OR7E37P | U |
| 6657 | chr6 | 43610802 | 43610931 | Gastric + Small Intes. + Colon Epithelium | 0.32 | 0.85 | Intergenic | RSPH9 | U |
| 6658 | chr13 | 110857417 | 110857907 | Gastric + Small Intes. + Colon Epithelium | 0.36 | 0.89 | intron | COL4A1 | U |
| 6659 | chr5 | 273394 | 273541 | Gastric + Small Intes. + Colon Epithelium | 0.3 | 0.82 | intron | PDCD6 | U |
| 6660 | chr19 | 45695965 | 45696130 | Gastric + Small Intes. + Colon Epithelium | 0.32 | 0.84 | Intergenic | BLOC1S3 | U |
| 6661 | chr17 | 71369565 | 71369751 | Gastric + Small Intes. + Colon Epithelium | 0.36 | 0.88 | intron | SDK2 | U |
| 6662 | chr17 | 79824280 | 79824466 | Gastric + Small Intes. + Colon Epithelium | 0.3 | 0.82 | Intergenic | ARHGDIA | U |
| 6663 | chr16 | 9002983 | 9003175 | Gastric + Small Intes. + Colon Epithelium | 0.38 | 0.9 | intron | USP7 | U |
| 6664 | chr11 | 126073383 | 126073607 | Gastric + Small Intes. + Colon Epithelium | 0.42 | 0.92 | exon, intron | RPUSD4, RPUSD4 | U |
| 6665 | chr1 | 1377044 | 1377106 | Gastric + Small Intes. + Colon Epithelium | 0.37 | 0.86 | Intergenic | LOC255512 | U |
| 6666 | chr17 | 70452771 | 70452885 | Gastric + Small Intes. + Colon Epithelium | 0.36 | 0.85 | intron | LINC00673 | U |
| 6667 | chr6 | 108170278 | 108170479 | Gastric + Small Intes. + Colon Epithelium | 0.34 | 0.82 | Intergenic | SCMLA | U |
| 6668 | chr16 | 85963633 | 85963905 | Gastric + Small Intes. + Colon Epithelium | 0.34 | 0.82 | Intergenic | IRF8 | U |
| 6669 | chr7 | 98764851 | 98764999 | Gastric + Small Intes. + Colon Epithelium | 0.34 | 0.81 | Intergenic | SMURF1 | U |
| 6670 | chr15 | 77867751 | 77867993 | Gastric + Small Intes. + Colon Epithelium | 0.36 | 0.82 | Intergenic | LINGO1 | U |
| 6671 | chr5 | 13524633 | 13524946 | Gastric + Small Intes. + Colon Epithelium | 0.37 | 0.82 | Intergenic | DNAH5 | U |
| 6672 | chr19 | 48106478 | 48106559 | Gastric + Small Intes. + Colon Epithelium | 0.36 | 0.8 | Intergenic | GLTSCR1 | U |
| 6673 | chr4 | 134189379 | 134189523 | Gastric + Small Intes. + Colon Epithelium | 0.32 | 0.8 | Intergenic | PCDH10 | U |
| 6674 | chr9 | 113028863 | 113028918 | Gastric + Small Intes. + Colon Epithelium | 0.11 | 0.89 | Intergenic | TXN | U |
| 6675 | chr1 | 19711383 | 19711578 | Gastric + Small Intes. + Colon Epithelium | 0.04 | 0.9 | intron | CAPZB | U |
| 6676 | chr20 | 9361360 | 9361544 | Gastric + Small Intes. + Colon Epithelium | 0.02 | 0.86 | intron | PLCB4 | U |
| 6677 | chr15 | 41842836 | 41842996 | Gastric + Small Intes. + Colon Epithelium | 0.11 | 0.9 | Intergenic | RPAP1 | U |
| 6678 | chr1 | 207182723 | 207182777 | Gastric + Small Intes. + Colon Epithelium | 0.12 | 0.9 | Intergenic | C1orf116 | U |
| 6679 | chr17 | 4144976 | 4145151 | Gastric + Small Intes. + Colon Epithelium | 0.09 | 0.87 | intron | ANKFY1 | U |
| 6680 | chr1 | 168116137 | 168116200 | Gastric + Small Intes. + Colon Epithelium | 0.14 | 0.85 | Intergenic | GPR161 | U |
| 6681 | chr9 | 34050582 | 34050733 | Gastric + Small Intes. + Colon Epithelium | 0.18 | 0.89 | Intergenic | UBAP2 | U |
| 6682 | chr14 | 74029365 | 74029432 | Gastric + Small Intes. + Colon Epithelium | 0.15 | 0.82 | Intergenic | HEATR4 | U |
| 6683 | chr14 | 77437547 | 77437695 | Gastric + Small Intes. + Colon Epithelium | 0.22 | 0.89 | Intergenic | IRF2BPL | U |
| 6684 | chr17 | 372114961450 | 37211875 | Gastric + Small Intes. + Colon Epithelium | 0.18 | 0.84 | Intergenic | LOC100131347 | U |
| 6685 | chr17 | 38844775 | 38844988 | Gastric + Small Intes. + Colon Epithelium | 0.22 | 0.86 | Intergenic | KRT24 | U |
| 6686 | chr20 | 516121 | 516341 | Gastric + Small Intes. + Colon Epithelium | 0.28 | 0.91 | intron | CSNK2A1 | U |
| 6687 | chr9 | 19336284 | 19336508 | Gastric + Small Intes. + Colon Epithelium | 0.36 | 0.93 | intron | DENND4C | U |
| 6688 | chr1 | 2157452091 | 215745274 | Gastric + Small Intes. + Colon Epithelium | 0.38 | 0.94 | intron | KCTD3 | U |
| 6689 | chr18 | 60675645 | 60675810 | Gastric + Small Intes. + Colon Epithelium | 0.3 | 0.86 | Intergenic | DHLPP1 | U |
| 6690 | chr9 | 45746113 | 45746368 | Gastric + Small Intes. + Colon Epithelium | 0.28 | 0.76 | Intergenic | FAM27A | U |
| 6691 | chr8 | 11553274 | 11553514 | Gastric + Small Intes. + Colon Epithelium | 0.59 | 0.14 | Intergenic | GATA4 | M |
| 6692 | chr5 | 7395230 | 7395525 | Gastric + Small Intes. + Colon Epithelium | 0.69 | 0.19 | promoter-TSS, Interg [symbol] | ADCY2, ADCY2 | M |
| 6693 | chr11 | 46317357 | 46317804 | Gastric + Small Intes. + Colon Epithelium | 0.56 | 0.06 | intron | CREB3L1 | M |
| 6694 | chr8 | 11554732 | 11555107 | Gastric + Small Intes. + Colon Epithelium | 0.58 | 0.12 | Intergenic | GATA4 | M |
| 6695 | chr16 | 87489775 | 87489952 | Small Intes. + Colon Epithelium | 0.01 | 0.94 | intron | ZCCHC14 | U |
| 6696 | chr21 | 45513359 | 45513531 | Small Intes. + Colon Epithelium | 0.03 | 0.95 | intron | TRAPPC10 | U |
| 6697 | chr11 | 75859077 | 75859219 | Small Intes. + Colon Epithelium | 0.04 | 0.94 | Intergenic | WNT11 | U |
| 6698 | chr18 | 21882532 | 21882889 | Small Intes. + Colon Epithelium | 0.04 | 0.94 | intron | OSBPL1A | U |
| 6699 | chr2 | 47595738 | 47595810 | Small Intes. + Colon Epithelium | 0.04 | 0.92 | promoter-TSS | EPCAM | U |
| 6700 | chr4 | 718410 | 718626 | Small Intes. + Colon Epithelium | 0.08 | 0.96 | intron | PCGF3 | U |
| 6701 | chr15 | 34371984 | 34372158 | Small Intes. + Colon Epithelium | 0.06 | 0.94 | Intergenic | EMC7 | U |
| 6702 | chr8 | 30633125 | 30633491 | Small Intes. + Colon Epithelium | 0.08 | 0.94 | Intergenic | UBXN8 | U |
| 6703 | chr3 | 1423223581 | 142322485 | Small Intes. + Colon Epithelium | 0.01 | 0.95 | intron | PLS1 | U |
| 6704 | chr2 | 200806812 | 200807037 | Small Intes. + Colon Epithelium | 0.02 | 0.94 | intron | TYWS | U |
| 6705 | chr7 | 11119454 | 19928 | Small Intes. + Colon Epithelium | 0.02 | 0.94 | intron | PHF14 | U |
| 6706 | chr3 | 67749797 | 67750071 | Small Intes. + Colon Epithelium | 0.02 | 0.93 | Intergenic | SUCLG2 | U |
| 6707 | chr21 | 290774031 | 29077476 | Small Intes. + Colon Epithelium | 0.02 | 0.92 | intron | MIR5009 | U |
| 6708 | chr17 | 26125232 | 26125407 | Small Intes. + Colon Epithelium | 0.02 | 0.92 | intron | NOS | U |
| 6709 | chr5 | 116183774 | 116183996 | Small Intes. + Colon Epithelium | 0.02 | 0.91 | Intergenic | SEMA6A | U |
| 6710 | chr13 | 22906573 | 22906832 | Small Intes. + Colon Epithelium | 0 | 0.89 | Intergenic | LINC00424 | U |
| 6711 | chr17 | 57813499 | 57813788 | Small Intes. + Colon Epithelium | 0.04 | 0.93 | intron | VMP1 | U |
| 6712 | chr11 | 128295328 | 128295628 | Small Intes. + Colon Epithelium | 0.04 | 0.93 | Intergenic | ETS1 | U |
| 6713 | chr5 | 160109768 | 160110199 | Small Intes. + Colon Epithelium | 0.01 | 0.9 | intron | ATP10B | U |
| 6714 | chr13 | 80016175 | 80016256 | Small Intes. + Colon Epithelium | 0.02 | 0.9 | Intergenic | RBM26-AS1 | U |
| 6715 | chr6 | 134689461 | 134689735 | Small Intes. + Colon Epithelium | 0.04 | 0.92 | Intergenic | LOC154092 | U |
| 6716 | chr15 | 83459321 | 83459397 | Small Intes. + Colon Epithelium | 0.02 | 0.89 | intron | FSDZ | U |
| 6717 | chr16 | 5113797 | 5113894 | Small Intes. + Colon Epithelium | 0.02 | 0.89 | intron | C16orf89 | U |
| 6718 | chr18 | 47040451 | 47040573 | Small Intes. + Colon Epithelium | 0.04 | 0.91 | Intergenic | RPL17-C18orf32 | U |
| 6719 | chr12 | 71547960 | 71548145 | Small Intes. + Colon Epithelium | 0.03 | 0.9 | intron | TSPAN8 | U |
| 6720 | chr1 | 207120022 | 207120270 | Small Intes. + Colon Epithelium | 0.04 | 0.91 | promoter-TSS | PIGR | U |
| 6721 | chr16 | 81606499 | 81606778 | Small Intes. + Colon Epithelium | 0.05 | 0.92 | intron | CMIP | U |
| 6722 | chr12 | 94464329 | 94464633 | Small Intes. + Colon Epithelium | 0.01 | 0.88 | Intergenic | PLXNC1 | U |
| 6723 | chr10 | 1304003261 | 130400612 | Small Intes. + Colon Epithelium | 0.02 | 0.88 | Intergenic | MKI67 | U |
| 6724 | chr2 | 122101306 | 122101610 | Small Intes. + Colon Epithelium | 0.07 | 0.93 | intron | CLASP1 | U |
| 6725 | chr12 | 14848427 | 14848820 | Small Intes. + Colon Epithelium | 0.02 | 0.88 | intron | GUCY2C | U |
| 6726 | chr17 | 2345980 | 2346376 | Small Intes. + Colon Epithelium | 0.07 | 0.93 | intron | METTL16 | U |
| 6727 | chr13 | 100339324 | 100339757 | Small Intes. + Colon Epithelium | 0.02 | 0.88 | intron | CLYBL | U |
| 6728 | chr19 | 2305151 | 2305259 | Small Intes. + Colon Epithelium | 0.02 | 0.87 | intron | LINGO3 | U |
| 6729 | chr4 | 27058679 | 27058847 | Small Intes. + Colon Epithelium | 0.01 | 0.86 | Intergenic | STIM2 | U |
| 6730 | chr2 | 47587863 | 47588077 | Small Intes. + Colon Epithelium | 0.06 | 0.91 | Intergenic | EPCAM | U |
| 6731 | chr10 | 12482435 | 12482701 | Small Intes. + Colon Epithelium | 0.1 | 0.95 | intron | CAMK1D | U |
| 6732 | chr5 | 177768752 | 177769022 | Small Intes. + Colon Epithelium | 0.02 | 0.87 | Intron | COL23A1 | U |
| 6733 | chr4 | 148614055 | 148614327 | Small Intes. + Colon Epithelium | 0.04 | 0.89 | Intergenic | PRMT10 | U |
| 6734 | chr7 | 101344576 | 101344950 | Small Intes. + Colon Epithelium | 0.04 | 0.89 | Intergenic | MYL10 | U |
| 6735 | chr2 | 169486471 | 169486750 | Small Intes. + Colon Epithelium | 0.09 | 0.93 | intron | CERS6 | U |
| 6736 | chr7 | 30296022 | 30296314 | Small Intes. + Colon Epithelium | 0.1 | 0.94 | Intergenic | ZNRF2 | U |
| 6737 | chr17 | 4948401 | 4948849 | Small Intes. + Colon Epithelium | 0.08 | 0.92 | Intergenic | SLC52A1 | U |
| 6738 | chr7 | 98844527 | 98844620 | Small Intes. + Colon Epithelium | 0.01 | 0.84 | Intergenic | MYH16 | U |
| 6739 | chr10 | 13379633 | 13379831 | Small Intes. + Colon Epithelium | 0.1 | 0.93 | intron | SEPHS1 | U |
| 6740 | chr10 | 59934716 | 59934921 | Small Intes. + Colon Epithelium | 0.02 | 0.85 | Intergenic | IPMK | U |
| 6741 | chr1 | 95082540 | 95082803 | Small Intes. + Colon Epithelium | 0.04 | 0.87 | Intergenic | F3 | U |
| 6742 | chr16 | 58349438 | 58349859 | Small Intes. + Colon Epithelium | 0.08 | 0.91 | Intergenic | PRSSS4 | U |
| 6743 | chr9 | 136837801 | 136837894 | Small Intes. + Colon Epithelium | 0.04 | 0.86 | intron | VAV2 | U |
| 6744 | chr17 | 79523884 | 79524201 | Small Intes. + Colon Epithelium | 0.06 | 0.88 | TTS | NPLOC4 | U |
| 6745 | chr4 | 175330079 | 175330561 | Small Intes. + Colon Epithelium | 0.04 | 0.86 | Intergenic | MIR4276 | U |
| 6746 | chr2 | 232768944 | 232769094 | Small Intes. + Colon Epithelium | 0.04 | 0.85 | Intergenic | MIR1471 | U |
| 6747 | chr21 | 43521772 | 43521926 | Small Intes. + Colon Epithelium | 0.08 | 0.89 | TTS | C21orf128 | U |
| 6748 | chr20 | 524553483 | 52455607 | Small Intes. + Colon Epithelium | 0.1 | 0.91 | Intergenic | SUMO1P1 | U |
| 6749 | chr19 | 47718897 | 47719372 | Small Intes. + Colon Epithelium | 0.1 | 0.91 | Intergenic | MIR3190 | U |
| 6750 | chr6 | 150253324 | 150253374 | Small Intes. + Colon Epithelium | 0.08 | 0.88 | Intergenic | RAET1G | U |
| 6751 | chr1 | 26503371 | 26503425 | Small Intes. + Colon Epithelium | 0.12 | 0.92 | promoter-TSS | CNKSR1 | U |
| 6752 | chr17 | 47060475 | 47060748 | Small Intes. + Colon Epithelium | 0.02 | 0.82 | Intergenic, Intergeni [symbol] ; | IGF28P1, GIP | U |
| 6753 | chr16 | 70318163 | 70318437 | Small Intes. + Colon Epithelium | 0.12 | 0.92 | Intron | AARS | U |
| 6754 | chr7 | 6406310 | 6406622 | Small Intes. + Colon Epithelium | 0.06 | 0.86 | Intergenic | RAC1 | U |
| 6755 | chr15 | 62870563 | 62870988 | Small Intes. + Colon Epithelium | 0.12 | 0.92 | Intergenic | MGC15885 | U |
| 6756 | chr10 | 30847753 | 30848064 | Small Intes. + Colon Epithelium | 0.12 | 0.9 | Intergenic | LYZL2 | U |
| 6757 | chr12 | 122590823 | 122591264 | Small Intes. + Colon Epithelium | 0.14 | 0.91 | intron | MLXIP | U |
| 6758 | chr11 | 118724393 | 118724487 | Small Intes. + Colon Epithelium | 0.18 | 0.93 | Intergenic | CXCR5 | U |
| 6759 | chr2 | 232532581 | 232532956 | Small Intes. + Colon Epithelium | 0.19 | 0.94 | Intergenic | PTMA | U |
| 6760 | chr22 | 46587735 | 46588099 | Small Intes. + Colon Epithelium | 0.26 | 0.95 | intron | PPARA | U |
| 6761 | chr10 | 99481763 | 99481814 | Small Intes. + Colon Epithelium | 0.01 | 0.95 | Intergenic | MARVELD1 | U |
| 6762 | chr19 | 2278616 | 2278708 | Small Intes. + Colon Epithelium | 0.01 | 0.94 | exon | C19orf35 | U |
| 6763 | chr14 | 55668352 | 55668536 | Small Intes. + Colon Epithelium | 0.01 | 0.93 | Intergenic | DLGAP5 | U |
| 6764 | chr19 | 39305863 | 39306273 | Small Intes. + Colon Epithelium | 0.01 | 0.93 | TTS, exon | ECH1 | U |
| 6765 | chr9 | 139497804 | 139498020 | Small Intes. + Colon Epithelium | 0.02 | 0.94 | Intergenic | EGFL7 | U |
| 6766 | chr6 | 2309359 | 2309763 | Small Intes. + Colon Epithelium | 0.03 | 0.95 | intron | LOC100508120 | U |
| 6767 | chr3 | 43655182 | 43655641 | Small Intes. + Colon Epithelium | 0.02 | 0.93 | intron | ANO10 | U |
| 6768 | chr17 | 80535107 | 80535364 | Small Intes. + Colon Epithelium | 0.06 | 0.96 | intron | FOXK2 | U |
| 6769 | chr17 | 80535398 | 80535833 | Small Intes. + Colon Epithelium | 0.05 | 0.95 | intron | FOXK2 | U |
| 6770 | chr20 | 57009379 | 57009561 | Small Intes. + Colon Epithelium | 0.04 | 0.93 | intron | VAPB | U |
| 6771 | chr10 | 1166110 | 1166313 | Small Intes. + Colon Epithelium | 0.07 | 0.96 | intron | WDR37 | U |
| 6772 | chr11 | 61148275 | 61148746 | Small Intes. + Colon Epithelium | 0.06 | 0.94 | Intergenic | TMEM216 | U |
| 6773 | chr1 | 53164855 | 53164931 | Small Intes. + Colon Epithelium | 0.06 | 0.93 | promoter-TSS | SELRC1 | U |
| 6774 | chr22 | 31062373 | 31062593 | Small Intes. + Colon Epithelium | 0.06 | 0.93 | intron | DUSP18 | U |
| 6775 | chr22 | 31076221 | 31076480 | Small Intes. + Colon Epithelium | 0.04 | 0.9 | Intergenic | DUSP18 | U |
| 6776 | chr11 | 6398940 | 6399034 | Small Intes. + Colon Epithelium | 0.09 | 0.93 | Intergenic | SMPD1 | U |
| 6777 | chr14 | 24611688 | 24611865 | Small Intes. + Colon Epithelium | 0.09 | 0.92 | promoter-TSS | EMC9 | U |
| 6778 | chr1 | 78458500 | 78458704 | Small Intes. + Colon Epithelium | 0.03 | 0.95 | Intergenic | DNAJB4 | U |
| 6779 | chr2 | 8849893 | 8850067 | Small Intes. + Colon Epithelium | 0.01 | 0.92 | Intergenic | ID2 | U |
| 6780 | chr2 | 47587206 | 47587387 | Small Intes. + Colon Epithelium | 0.02 | 0.92 | Intergenic | EPCAM | U |
| 6781 | chr22 | 20857395 | 20857653 | Small Intes. + Colon Epithelium | 0.03 | 0.93 | Intergenic | MED15 | U |
| 6782 | chr15 | 40645207 | 40645581 | Small Intes. + Colon Epithelium | 0.01 | 0.91 | intron | PHGR1 | U |
| 6783 | chr2 | 97427379 | 97427487 | Small Intes. + Colon Epithelium | 0.01 | 0.9 | exon | CNNM4 | U |
| 6784 | chr2 | 106959820 | 106960122 | Small Intes. + Colon Epithelium | 0.01 | 0.9 | Intergenic | PLGLA | U |
| 6785 | chr5 | 151251315 | 151251432 | Small Intes. + Colon Epithelium | 0.03 | 0.91 | intron | GLRA1 | U |
| 6786 | chr17 | 17466693 | 17467012 | Small Intes. + Colon Epithelium | 0.06 | 0.94 | intron | PEMT | U |
| 6787 | chr14 | 73238101 | 73238561 | Small Intes. + Colon Epithelium | 0.02 | 0.9 | intron | DPF3 | U |
| 6788 | chr20 | 42852813 | 42852870 | Small Intes. + Colon Epithelium | 0.04 | 0.91 | intron | LOC100505783 | U |
| 6789 | chr2 | 28718664 | 28718829 | Small Intes. + Colon Epithelium | 0.03 | 0.9 | promoter-TSS | PLB1 | U |
| 6790 | chr9 | 112069228 | 112069552 | Small Intes. + Colon Epithelium | 0.03 | 0.9 | intron | EPB41L4B | U |
| 6791 | chr17 | 73613196 | 73613594 | Small Intes. + Colon Epithelium | 0.02 | 0.89 | intron | MYO158 | U |
| 6792 | chr17 | 77074568 | 77075062 | Small Intes. + Colon Epithelium | 0.04 | 0.91 | intron | ENGASE | U |
| 6793 | chr9 | 18683283 | 18683451 | Small Intes. + Colon Epithelium | 0.04 | 0.9 | intron | ADAMTSL1 | U |
| 6794 | chr6 | 12262875 | 12263062 | Small Intes. + Colon Epithelium | 0.03 | 0.89 | Intergenic | EDN1 | U |
| 6795 | chr4 | 2038234 | 2038431 | Small Intes. + Colon Epithelium | 0.01 | 0.87 | Intergenic | C4orf48 | U |
| 6796 | chr20 | 52285405 | 52285721 | Small Intes. + Colon Epithelium | 0.03 | 0.89 | Intergenic | ZNF217 | U |
| 6797 | chr2 | 239320905 | 239321299 | Small Intes. + Colon Epithelium | 0.08 | 0.94 | Intergenic | ASB1 | U |
| 6798 | chr9 | 79345539 | 79345600 | Small Intes. + Colon Epithelium | 0.02 | 0.87 | intron | PRUNE2 | U |
| 6799 | chr11 | 6398960 | 6399035 | Small Intes. + Colon Epithelium | 0.08 | 0.93 | Intergenic | SMPD1 | U |
| 6800 | chr20 | 42982934 | 42983084 | Small Intes. + Colon Epithelium | 0.02 | 0.87 | Intergenic | R3HDML | U |
| 6801 | chr20 | 38304946 | 38305151 | Small Intes. + Colon Epithelium | 0.02 | 0.87 | Intergenic | LOC339568 | U |
| 6802 | chr1 | 3431025 | 3431253 | Small Intes. + Colon Epithelium | 0.04 | 0.89 | intron, exon | MEGF6, MEGF6 | U |
| 6803 | chr9 | 110368576 | 110368851 | Small Intes. + Colon Epithelium | 0.02 | 0.87 | Intergenic | KLF4 | U |
| 6804 | chr1 | 6422485 | 6422838 | Small Intes. + Colon Epithelium | 0.05 | 0.9 | intron | ACOT7 | U |
| 6805 | chr19 | 39306298 | 39306385 | Small Intes. + Colon Epithelium | 0.02 | 0.86 | exon, intron | ECH1, ECH1 | U |
| 6806 | chr10 | 97322759 | 97322853 | Small Intes. + Colon Epithelium | 0.01 | 0.85 | Intergenic | SORBS1 | U |
| 6807 | chr20 | 49969364 | 49969486 | Small Intes. + Colon Epithelium | 0.09 | 0.93 | Intergenic | MIR3194 | U |
| 6808 | chr9 | 114247470 | 114247605 | Small Intes. + Colon Epithelium | 0.09 | 0.93 | promoter-TSS | KIAA0368 | U |
| 6809 | chr19 | 4054373 | 4054680 | Small Intes. + Colon Epithelium | 0.09 | 0.93 | exon | ZBTB7A | U |
| 6810 | chr5 | 149547488 | 149547864 | Small Intes. + Colon Epithelium | 0.01 | 0.85 | intron | CDX1 | U |
| 6811 | chr22 | 25324641 | 25324754 | Small Intes. + Colon Epithelium | 0.02 | 0.85 | Intergenic | TMEM211 | U |
| 6812 | chr19 | 552942 | 553082 | Small Intes. + Colon Epithelium | 0.02 | 0.85 | Intergenic | GZMM | U |
| 6813 | chr4 | 965124 | 965325 | Small Intes. + Colon Epithelium | 0.08 | 0.9 | intron | DGKQ | U |
| 6814 | chr7 | 1563550 | 1563709 | Small Intes. + Colon Epithelium | 0.02 | 0.82 | Intergenic | MAFK | U |
| 6815 | chr14 | 105723681 | 105723909 | Small Intes. + Colon Epithelium | 0.14 | 0.94 | intron | BRF1 | U |
| 6816 | chr4 | 1015911 | 1016203 | Small Intes. + Colon Epithelium | 0.14 | 0.94 | exon, intron | FGFRL1, FGFRL1 | U |
| 6817 | chr11 | 864528 | 864847 | Small Intes. + Colon Epithelium | 0.1 | 0.9 | intron | TSPAN4 | U |
| 6818 | chr20 | 42982573 | 42982873 | Small Intes. + Colon Epithelium | 0.02 | 0.81 | Intergenic | R3HDMI | U |
| 6819 | chr10 | 74451042 | 74451160 | Small Intes. + Colon Epithelium | 0.12 | 0.9 | promoter-TSS | MCU | U |
| 6820 | chr6 | 71121859 | 71122005 | Small Intes. + Colon Epithelium | 0.16 | 0.94 | Intergenic | FAM135A | U |
| 6821 | chr6 | 42521871 | 42521982 | Small Intes. + Colon Epithelium | 0.96 | 0.15 | Intergenic | UBR2 | M |
| 6822 | chr10 | 112838797 | 112838831 | Small Intes. + Colon Epithelium | 0.96 | 0.19 | exon | ADRA2A | M |
| 6823 | chr2 | 202900483 | 202900532 | Small Intes. + Colon Epithelium | 0.92 | 0.21 | exon | FZD7 | M |
| 6824 | chr9 | 140189496 | 140189574 | Small Intes. + Colon Epithelium | 0.78 | 0.07 | Intergenic | NRARP | M |
| 6825 | chr19 | 13813350 | 13813427 | Small Intes. + Colon Epithelium | 0.85 | 0.2 | Intergenic | CCDC130 | M |
| 6826 | chr4 | 94755859 | 94755934 | Small Intes. + Colon Epithelium | 0.96 | 0.08 | Intergenic | ATOH1 | M |
| 6827 | chr2 | 114034961 | 114035252 | Small Intes. + Colon Epithelium | 0.93 | 0.08 | intron | PAX8 | M |
| 6828 | chr1 | 226814277 | 226814420 | Small Intes. + Colon Epithelium | 0.87 | 0.03 | Intergenic | C1orf95 | M |
| 6829 | chr4 | 94755553 | 94755790 | Small Intes. + Colon Epithelium | 0.96 | 0.12 | Intergenic | ATOH1 | M |
| 6830 | chr10 | 112838336 | 112838406 | Small Intes. + Colon Epithelium | 0.96 | 0.13 | exon | ADRAZA | M |
| 6831 | chr6 | 26017646 | 26018119 | Small Intes. + Colon Epithelium | 0.93 | 0.1 | exon | HIST1H1A | M |
| 6832 | chr2 | 114035273 | 114035466 | Small Intes. + Colon Epithelium | 0.94 | 0.13 | intron | PAX8 | M |
| 6833 | chr2 | 114034646 | 114034958 | Small Intes. + Colon Epithelium | 0.92 | 0.11 | intron | PAX8 | M |
| 6834 | chr3 | 194118116 | 194118190 | Small Intes. + Colon Epithelium | 0.92 | 0.12 | exon | GP5 | M |
| 6835 | chr10 | 112838475 | 112838642 | Small Intes. + Colon Epithelium | 0.96 | 0.16 | exon | ADRA2A | M |
| 6836 | chr2 | 25500163 | 25500287 | Small Intes. + Colon Epithelium | 0.92 | 0.13 | intron | DNMT3A | M |
| 6837 | chr8 | 27449792 | 27450108 | Small Intes. + Colon Epithelium | 0.84 | 0.05 | Intergenic | CLU | M |
| 6838 | chr2 | 233793224 | 233793418 | Small Intes. + Colon Epithelium | 0.92 | 0.15 | promoter-TSS | NGEF | M |
| 6839 | chr7 | 329232 | 329477 | Small Intes. + Colon Epithelium | 0.93 | 0.16 | promoter-TSS | LOC100288524 | M |
| 6840 | chr18 | 45058273 | 45058297 | Small Intes. + Colon Epithelium | 0.9 | 0.16 | Intergenic | IER3IP1 | M |
| 6841 | chr10 | 112838267 | 112838334 | Small Intes. + Colon Epithelium | 0.96 | 0.22 | exon | ADRA2ZA | M |
| 6842 | chr14 | 21094173 | 21094366 | Small Intes. + Colon Epithelium | 0.84 | 0.12 | Intergenic | OR6S31 | M |
| 6843 | chr6 | 27473456 | 27473634 | Small Intes. + Colon Epithelium | 0.82 | 0.11 | Intergenic | ZNF184 | M |
| 6844 | chr19 | 37096487 | 37096817 | Small Intes. + Colon Epithelium | 0.74 | 0.03 | promoter-TSS, intro [symbol] | ZNF529, ZNF382 | M |
| 6845 | chr12 | 62585454 | 62585504 | Small Intes. + Colon Epithelium | 0.76 | 0.1 | exon | FAM19A2 | M |
| 6846 | chr13 | 108206339 | 108206532 | Colon + Heart Fibroblasts | 0.23 | 0.85 | intron | FAM155A | U |
| 6847 | chr2 | 75765726 | 75766101 | Colon + Heart Fibroblasts | 0.27 | 0.86 | intron | EVA1A | U |
| 6848 | chr1 | 67873294 | 67873605 | Colon + Heart Fibroblasts | 0.31 | 0.89 | exon | SERBP1 | U |
| 6849 | chr1 | 213887598 | 213887905 | Colon + Heart Fibroblasts | 0.31 | 0.87 | Intergenic | LINC00538 | U |
| 6850 | chr13 | 113185690 | 113185779 | Colon + Heart Fibroblasts | 0.36 | 0.91 | intron | TUBGCP3 | U |
| 6851 | chr7 | 40940368 | 90940824 | Colon + Heart Fibroblasts | 0.25 | 0.8 | Intergenic | FZD1 | U |
| 6852 | chr15 | 68705786 | 68706249 | Colon + Heart Fibroblasts | 0.33 | 0.88 | intron | ITGA11 | U |
| 6853 | chr8 | 28302585 | 28302736 | Colon + Heart Fibroblasts | 0.34 | 0.88 | intron | FBXO16 | U |
| 6854 | chr2 | 8110088 | 8110287 | Colon + Heart Fibroblasts | 0.31 | 0.85 | intron | LOC339788 | U |
| 6855 | chr4 | 70391691 | 70391750 | Colon + Heart Fibroblasts | 0.34 | 0.86 | Intergenic | UGT284 | U |
| 6856 | chr6 | 101175196 | 101175306 | Colon + Heart Fibroblasts | 0.38 | 0.9 | intron | ASCC3 | U |
| 6857 | chr11 | 8378249 | 8378404 | Colon + Heart Fibroblasts | 0.31 | 0.83 | Intergenic | LMO1 | U |
| 6858 | chr16 | 88953123 | 88953235 | Colon + Heart Fibroblasts | 0.35 | 0.86 | intron | CBFA2T3 | U |
| 6859 | chr10 | 1090408171 | 109041072 | Colon + Heart Fibroblasts | 0.31 | 0.82 | Intergenic | SORCS1 | U |
| 6860 | chr10 | 72989131 | 72989570 | Colon + Heart Fibroblasts | 0.34 | 0.84 | intron | UNC5B | U |
| 6861 | chr11 | 12816156 | 12816415 | Colon + Heart Fibroblasts | 0.37 | 0.85 | intron | TEAD1 | U |
| 6862 | chr5 | 39380163 | 39380582 | Colon + Heart Fibroblasts | 0.37 | 0.83 | Intron | DAB2 | U |
| 6863 | chr | 68415027 | 68415155 | Colon + Heart Fibroblasts | 0.35 | 0.8 | Intergenic | PIA1 | U |
| 6864 | chr14 | 96701161 | 96701223 | Colon + Heart Fibroblasts | 0.28 | 0.88 | intron | BDKRB2 | U |
| 6865 | chr2 | 33453062 | 33453168 | Colon + Heart Fibroblasts | 0.36 | 0.88 | intron | LTBP1 | U |
| 6866 | chr10 | 34370079 | 34370308 | Colon + Heart Fibroblasts | 0.38 | 0.88 | Intergenic | LINC00838 | U |
| 6867 | chr3 | 10997594 | 10997775 | Colon + Heart Fibroblasts | 0.32 | 0.79 | Intergenic | SLC6A | U |
| 6868 | chr20 | 59234431 | 59234489 | Colon + Heart Fibroblasts | 0.39 | 0.85 | Intergenic | MIR4533 | U |
| 6869 | chr5 | 3269626 | 3269743 | Colon + Heart Fibroblasts | 0.43 | 0.8 | Intergenic | LOC285577 | U |
| 6870 | chr5 | 38059911 | 38060133 | Colon + Heart Fibroblasts | 0.24 | 0.87 | Intergenic | GDNF | U |
| 6871 | chr21 | 46994499 | 46994773 | Colon + Heart Fibroblasts | 0.3 | 0.84 | Intergenic | SLC19A1 | U |
| 6872 | chr7 | 134136965 | 134137121 | Colon + Heart Fibroblasts | 0.34 | 0.87 | intron | AKR181 | U |
| 6873 | chr14 | 39474369 | 39474677 | Colon + Heart Fibroblasts | 0.24 | 0.89 | Intergenic | LINC00639 | U |
| 6874 | chr6 | 14865485 | 14865639 | Colon + Heart Fibroblasts | 0.24 | 0.88 | Intergenic | JARID2 | U |
| 6875 | chr20 | 31198437 | 31198668 | Colon + Heart Fibroblasts | 0.28 | 0.85 | Intergenic | LOC149950 | U |
| 6876 | chr1 | 54818464 | 54818653 | Colon + Heart Fibroblasts | 0.31 | 0.87 | intron | SSBP3 | U |
| 6877 | chr1 | 235973132 | 235973209 | Colon + Heart Fibroblasts | 0.32 | 0.88 | exon | LYST | U |
| 6878 | chr7 | 4119888 | 4120145 | Colon + Heart Fibroblasts | 0.39 | 0.85 | intron | SDK1 | U |
| 6879 | chr9 | 137393746 | 137393832 | Colon + Heart Fibroblasts | 0.65 | 0.16 | Intergenic | MIR4669 | M |
| 6880 | chr4 | 174437547 | 174437671 | Colon + Heart Fibroblasts | 0.71 | 0.24 | Intergenic | HAND2 | M |
| 6881 | chr6 | 32063895 | 32063950 | Colon + Heart Fibroblasts | 0.72 | 0.29 | intron | TNXB | M |
| 6882 | chr3 | 1340940441 | 134094236 | Colon + Heart Fibroblasts | 0.58 | 0.15 | promoter-TSS | AMOTL2 | M |
| 6883 | chr1 | 1289240 | 1289556 | Colon + Heart Fibroblasts | 0.62 | 0.21 | intron | MXRA8 | M |
| 6884 | chr6 | 32064206 | 32064259 | Colon + Heart Fibroblasts | 0.63 | 0.25 | intron | TNXB | M |
| 6885 | chr9 | 137393398 | 137393625 | Colon + Heart Fibroblasts | 0.6 | 0.23 | Intergenic | MIR4669 | M |
| 6886 | chr1 | 233751029 | 233751089 | Colon + Heart Fibroblasts | 0.55 | 0.19 | intron | KCNK1 | M |
| 6887 | chr6 | 32064082 | 32064120 | Colon + Heart Fibroblasts | 0.62 | 0.28 | intron | TNXB | M |
| 6888 | chr22 | 45148085 | 45148197 | Colon + Heart Fibroblasts | 0.57 | 0.23 | promoter-TSS | ARHGAP8 | M |
| 6889 | chr2 | 220406860 | 220407000 | Colon + Heart Fibroblasts | 0.53 | 0.2 | intron | CHPP | M |
| 6890 | chr14 | 95757675 | 95757926 | Colon + Heart Fibroblasts | 0.56 | 0.23 | intron | CLMN | M |
| 6891 | chr21 | 46331292 | 46331607 | Colon + Heart Fibroblasts | 0.62 | 0.13 | intron | ITGB2 | M |
| 6892 | chr8 | 102505384 | 102505561 | Colon + Heart Fibroblasts | 0.6 | 0.16 | intron | GRHL2 | M |
| 6893 | chr19 | 31847840 | 31848086 | Colon + Heart Fibroblasts | 0.57 | 0.15 | Intergenic | TSHZ3 | M |
| 6894 | chr22 | 29400655 | 29400917 | Colon + Heart Fibroblasts | 0.59 | 0.17 | intron | ZNRF3 | M |
| 6895 | chr14 | 100126575 | 100126712 | Colon + Heart Fibroblasts | 0.61 | 0.21 | exon | HHIPL1 | M |
| 6896 | chr5 | 122422278 | 122422527 | Colon + Heart Fibroblasts | 0.53 | 0.18 | Intergenic | PRDM6 | M |
| 6897 | chr19 | 10077107 | 10077444 | Colon + Heart Fibroblasts | 0.56 | 0.21 | exon, intron | COLSA3, COL5A3 | M |
| 6898 | chr5 | 122434244 | 122434305 | Colon + Heart Fibroblasts | 0.53 | 0.22 | intron | PRDM6 | M |
| 6899 | chr13 | 103353210 | 103353268 | Cardiomyocytes + Skeletal + Smooth muscle cells | 0.21 | 0.89 | Intergenic | METTL21C | U |
| 6900 | chr7 | 139596699 | 139596859 | Cardiomyocytes + Skeletal + Smooth muscle cells | 0.19 | 0.85 | intron | TBXAS1 | U |
| 6901 | chr12 | 109182411 | 109182583 | Cardiomyocytes + Skeletal + Smooth muscle cells | 0.23 | 0.88 | exon | SSH1 | U |
| 6902 | chr3 | 194577074 | 194577478 | Cardiomyocytes + Skeletal + Smooth muscle cells | 0.23 | 0.87 | Intergenic | LOC100507391 | U |
| 6903 | chr6 | 154931253 | 154931684 | Cardiomyocytes + Skeletal + Smooth muscle cells | 0.25 | 0.89 | Intergenic | CNKSR3 | U |
| 6904 | chr3 | 75428327 | 75428518 | Cardiomyocytes + Skeletal + Smooth muscle cells | 0.2 | 0.81 | Intergenic | FAM86DP | U |
| 6905 | chr1 | 6125551 | 6125711 | Cardiomyocytes + Skeletal + Smooth muscle cells | 0.26 | 0.85 | intron | KCNAB2 | U |
| 6906 | chr1 | 208568164 | 208568368 | Cardiomyocytes + Skeletal + Smooth muscle cells | 0.25 | 0.82 | Intergenic | PLXNA2 | U |
| 6907 | chr12 | 94659279 | 94659540 | Cardiomyocytes + Skeletal + Smooth muscle cells | 0.33 | 0.82 | intron | PLXNC1 | U |
| 6908 | chr3 | 14564094 | 14564245 | Cardiomyocytes + Skeletal + Smooth muscle cells | 0.2 | 0.85 | intron | GRIP2 | U |
| 6909 | chr4 | 169561224 | 169561408 | Cardiomyocytes + Skeletal + Smooth muscle cells | 0.34 | 0.91 | intron | PALLD | U |
| 6910 | chr1 | 210407630 | 210407821 | Cardiomyocytes + Skeletal + Smooth muscle cells | 0.59 | 0.11 | promoter-TSS | SERTAD4-AS1 | U |
| 6911 | chr1 | 109104511 | 109104644 | Cardiomyocytes + Skeletal + Smooth muscle cells | 0.59 | 0.16 | intron | FAM102B | U |
| 6912 | chr17 | 65517833 | 65517989 | Skeletal + Smooth muscle cells | 0.21 | 0.87 | intron | PITPNC1 | U |
| 6913 | chr4 | 129315804 | 129316237 | Skeletal + Smooth muscle cells | 0.29 | 0.87 | Intergenic | PGRMC2 | U |
| 6914 | chr11 | 39346878 | 39347309 | Skeletal + Smooth muscle cells | 0.24 | 0.82 | Intergenic | LRRC4C | U |
| 6915 | chr12 | 124611762 | 124611965 | Skeletal + Smooth muscle cells | 0.35 | 0.91 | intron | ZNF664-FAM101A | U |
| 6916 | chr7 | 36254259 | 36254475 | Skeletal + Smooth muscle cells | 0.33 | 0.88 | intron | EEPD1 | U |
| 6917 | chr12 | 65100782 | 65101169 | Skeletal + Smooth muscle cells | 0.35 | 0.9 | Intergenic | IGNS | U |
| 6918 | chr17 | 80357575 | 80357722 | Skeletal + Smooth muscle cells | 0.28 | 0.83 | intron | OGFOD3 | U |
| 6919 | chrX | 9644602 | 9644654 | Skeletal + Smooth muscle cells | 0.37 | 0.9 | intron | TBL1X | U |
| 6920 | chr8 | 77690804 | 77691189 | Skeletal + Smooth muscle cells | 0.34 | 0.87 | intron | ZFHX4 | U |
| 6921 | chr7 | 33540721 | 33541122 | Skeletal + Smooth muscle cells | 0.37 | 0.89 | intron | BB59 | U |
| 6922 | chr7 | 139226844 | 139226943 | Skeletal + Smooth muscle cells | 0.27 | 0.78 | intron | CLEC2L | U |
| 6923 | chr2 | 51530026 | 51530321 | Skeletal + Smooth muscle cells | 0.34 | 0.84 | Intergenic | NRXN1 | U |
| 6924 | chr3 | 129817943 | 129818176 | Skeletal + Smooth muscle cells | 0.35 | 0.84 | TTS | ALG1L2 | U |
| 6925 | chr1 | 183701573 | 183701994 | Skeletal + Smooth muscle cells | 0.36 | 0.85 | intron | RGL1 | U |
| 6926 | chr2 | 240228774 | 240228845 | Skeletal + Smooth muscle cells | 0.4 | 0.88 | intron | HDAC4 | U |
| 6927 | chr11 | 132582808 | 132582929 | Skeletal + Smooth muscle cells | 0.38 | 0.86 | Intron | OPCML | U |
| 6928 | chr1 | 114525871 | 114526074 | Skeletal + Smooth muscle cells | 0.34 | 0.8 | Intergenic | OLFML3 | U |
| 6929 | chr7 | 74245167 | 74245481 | Skeletal + Smooth muscle cells | 0.34 | 0.6 | intron | GTF2IRD2 | U |
| 6930 | chr7 | 68683058 | 68683273 | Skeletal + Smooth muscle cells | 0.28 | 0.81 | Intergenic | AUTS2 | U |
| 6931 | chr20 | 24763072 | 24763358 | Skeletal + Smooth muscle cells | 0.29 | 0.83 | Intergenic | CST7 | U |
| 6932 | chr8 | 26048065 | 26048282 | Skeletal + Smooth muscle cells | 0.69 | 0.15 | Intergenic | PPP2R2A | M |
| 6933 | chr6 | 75794646 | 75795088 | Skeletal + Smooth muscle cells | 0.63 | 0.13 | exon | COL12A1 | M |
| 6934 | chr20 | 3654323 | 3654392 | Skeletal + Smooth muscle cells | 0.66 | 0.23 | intron, exon | ADAM33, ADAM33 | M |
| 6935 | chr9 | 137393398 | 137393625 | Skeletal + Smooth muscle cells | 0.65 | 0.22 | Intergenic | MIR4669 | M |
| 6936 | chr2 | 220406860 | 220407000 | Skeletal + Smooth muscle cells | 0.6 | 0.2 | intron | CHPF | M |
| 6937 | chr20 | 3653191 | 3653331 | Skeletal + Smooth muscle cells | 0.72 | 0.13 | exon | ADAM33 | M |
| 6938 | chr20 | 3653350 | 3653771 | Skeletal + Smooth muscle cells | 0.62 | 0.1 | intron, exon | ADAM33, ADAM33 | M |
| 6939 | chr20 | 3653926 | 3654003 | Skeletal + Smooth muscle cells | 0.64 | 0.21 | intron, exon | ADAM33, ADAM33 | M |
| 6940 | chr12 | 124692480 | 124692529 | Heart Cardiomyocytes + Fibroblasts | 0.04 | 0.95 | intron | ZNF664 [symbol] | U |
| 6941 | chr2 | 204995100 | 204995147 | Heart Cardiomyocytes + Fibroblasts | 0.09 | 0.94 | Intergenic | ICOS | U |
| 6942 | chr18 | 6143914 | 6144023 | Heart Cardiomyocytes + Fibroblasts | 0.09 | 0.93 | intron | L3MBTL4 | U |
| 6943 | chr2 | 233839348 | 233839401 | Heart Cardiomyocytes + Fibroblasts | 0.06 | 0.89 | exon | NGEF | U |
| 6944 | chr13 | 30098260 | 30098504 | Heart Cardiomyocytes + Fibroblasts | 0.1 | 0.93 | exon | ISLC7A1 | U |
| 6945 | chr10 | 52440841 | 52441024 | Heart Cardiomyocytes + Fibroblasts | 0.06 | 0.88 | Intergenic | SGMS1 | U |
| 6946 | chr11 | 116728737 | 116729029 | Heart Cardiomyocytes + Fibroblasts | 0.06 | 0.86 | exon | SIK3 | U |
| 6947 | chr7 | 150489478 | 150489834 | Heart Cardiomyocytes + Fibroblasts | 0.08 | 0.88 | intron | TMEM176B | U |
| 6948 | chr1 | 205811634 | 205811974 | Heart Cardiomyocytes + Fibroblasts | 0.09 | 0.88 | exon, intron | PM20D1 | U |
| 6949 | chr8 | 36788587 | 36788945 | Heart Cardiomyocytes + Fibroblasts | 0.12 | 0.9 | intron | KCNU1 | U |
| 6950 | chr6 | 164265488 | 164265690 | Heart Cardiomyocytes + Fibroblasts | 0.15 | 0.91 | Intergenic | QKI | U |
| 6951 | chr17 | 78846018 | 78846244 | Heart Cardiomyocytes + Fibroblasts | 0.11 | 0.86 | intron | RPTOR | U |
| 6952 | chr7 | 26654405 | 26654811 | Heart Cardiomyocytes + Fibroblasts | 0.17 | 0.92 | Intergenic | C7orf71 | U |
| 6953 | chr6 | 164526953 | 164527096 | Heart Cardiomyocytes + Fibroblasts | 0.12 | 0.85 | Intergenic | QKI | U |
| 6954 | chr1 | 44715613 | 44715807 | Heart Cardiomyocytes + Fibroblasts | 0.17 | 0.9 | intron | ERI3 | U |
| 6955 | chr16 | 4014051 | 4014252 | Heart Cardiomyocytes + Fibroblasts | 0.16 | 0.89 | exon | ADCY9 | U |
| 6956 | chr21 | 44738312 | 44738466 | Heart Cardiomyocytes + Fibroblasts | 0.21 | 0.93 | Intergenic | SIK1 | U |
| 6957 | chr6 | 106512194 | 106512445 | Heart Cardiomyocytes + Fibroblasts | 0.23 | 0.94 | Intergenic | PRDM1 | U |
| 6958 | chr17 | 74694601 | 74695045 | Heart Cardiomyocytes + Fibroblasts | 0.22 | 0.91 | intron | MXRA7 | U |
| 6959 | chr4 | 3912014 | 3912191 | Heart Cardiomyocytes + Fibroblasts | 0.17 | 0.86 | Intergenic | FAM86EP | U |
| 6960 | chr10 | 86919042 | 86919080 | Heart Cardiomyocytes + Fibroblasts | 0.1 | 0.92 | Intergenic | GRID1-AS1 | U |
| 6961 | chr12 | 124692706 | 124692822 | Heart Cardiomyocytes + Fibroblasts | 0.04 | 0.86 | intron | ZNF664-FAM101A | U |
| 6962 | chr4 | 183936178 | 183936350 | Heart Cardiomyocytes + Fibroblasts | 0.06 | 0.88 | Intergenic | FAM92A1P2 | U |
| 6963 | chr2 | 204994800 | 204995012 | Heart Cardiomyocytes + Fibroblasts | 0.04 | 0.86 | Intergenic | ICOS | U |
| 6964 | chr10 | 132942326 | 132942369 | Heart Cardiomyocytes + Fibroblasts | 0.06 | 0.87 | intron | TCERG1L | U |
| 6965 | chr8 | 61350445 | 61350879 | Heart Cardiomyocytes + Fibroblasts | 0.1 | 0.88 | Intergenic | RAB2A | U |
| 6966 | chr3 | 64579929 | 64580059 | Heart Cardiomyocytes + Fibroblasts | 0.12 | 0.89 | exon | ADAMTS9 | U |
| 6967 | chr10 | 68943889 | 68944213 | Heart Cardiomyocytes + Fibroblasts | 0.12 | 0.89 | intron | CTNNA3 | U |
| 6968 | chr10 | 132941935 | 132942020 | Heart Cardiomyocytes + Fibroblasts | 0.13 | 0.89 | intron | TCERG1L | U |
| 6969 | chr17 | 74365884 | 74366060 | Heart Cardiomyocytes + Fibroblasts | 0.07 | 0.83 | Intergenic | SPHK1 | U |
| 6970 | chr6 | 74281208 | 74281364 | Heart Cardiomyocytes + Fibroblasts | 0.14 | 0.89 | Intergenic | EEF1A1 | U |
| 6971 | chr6 | 56485141 | 56485569 | Heart Cardiomyocytes + Fibroblasts | 0.15 | 0.89 | exon | DST | U |
| 6972 | chr1 | 30393725 | 30393757 | Heart Cardiomyocytes + Fibroblasts | 0.13 | 0.86 | Intergenic | MATN1-AS1 | U |
| 6973 | chr8 | 57111069 | 57111218 | Heart Cardiomyocytes + Fibroblasts | 0.12 | 0.85 | intron | PLAG1 | U |
| 6974 | chr1 | 19383637 | 19383929 | Heart Cardiomyocytes + Fibroblasts | 0.13 | 0.86 | Intergenic | IFFO2 | U |
| 6975 | chr14 | 96351106 | 96351163 | Heart Cardiomyocytes + Fibroblasts | 0.15 | 0.87 | intron | LINC00617 | U |
| 6976 | chr10 | 77495522 | 77495676 | Heart Cardiomyocytes + Fibroblasts | 0.19 | 0.91 | Intergenic | C10orf11 | U |
| 6977 | chr18 | 46268834 | 46269008 | Heart Cardiomyocytes + Fibroblasts | 0.15 | 0.87 | intron | CTIF | U |
| 6978 | chr1 | 48231156 | 48231353 | Heart Cardiomyocytes + Fibroblasts | 0.15 | 0.87 | exon | TRABD2B | U |
| 6979 | chr6 | 110733693 | 110733896 | Heart Cardiomyocytes + Fibroblasts | 0.12 | 0.84 | intron | DDO | U |
| 6980 | chr3 | 14598605 | 14598779 | Heart Cardiomyocytes + Fibroblasts | 0.17 | 0.88 | Intergenic | GRIP2 | U |
| 6981 | chr17 | 41430132 | 41430356 | Heart Cardiomyocytes + Fibroblasts | 0.14 | 0.85 | Intergenic | LOC100130581 | U |
| 6982 | chr13 | 74557243 | 74557607 | Heart Cardiomyocytes + Fibroblasts | 0.18 | 0.89 | intron | KLF12 | U |
| 6983 | chr5 | 107585052 | 107585456 | Heart Cardiomyocytes + Fibroblasts | 0.18 | 0.89 | intron | FBXL17 | U |
| 6984 | chr3 | 4714755 | 4714902 | Heart Cardiomyocytes + Fibroblasts | 0.16 | 0.86 | exon | ITPR1 | U |
| 6985 | chr10 | 78203058 | 78203213 | Heart Cardiomyocytes + Fibroblasts | 0.24 | 0.94 | intron | C10orf11 | U |
| 6986 | chr1 | 81845626 | 81845783 | Heart Cardiomyocytes + Fibroblasts | 0.16 | 0.86 | Intergenic | LPHN2 | U |
| 6987 | chr5 | 147832067 | 147832514 | Heart Cardiomyocytes + Fibroblasts | 0.2 | 0.9 | intron | HTR4 | U |
| 6988 | chr5 | 124133353 | 124133751 | Heart Cardiomyocytes + Fibroblasts | 0.22 | 0.91 | Intergenic | ZNF608 | U |
| 6989 | chr11 | 131369292 | 131369352 | Heart Cardiomyocytes + Fibroblasts | 0.17 | 0.85 | intron | NTM | U |
| 6990 | chr10 | 88973836 | 88973928 | Heart Cardiomyocytes + Fibroblasts | 0.26 | 0.94 | Intergenic | FAM22A | U |
| 6991 | chr5 | 136617762 | 136617903 | Heart Cardiomyocytes + Fibroblasts | 0.18 | 0.86 | intron | SPOCK1 | U |
| 6992 | chr10 | 112444634 | 112444853 | Heart Cardiomyocytes + Fibroblasts | 0.17 | 0.85 | intron | RBM20 | U |
| 6993 | chr4 | 182193747 | 182194100 | Heart Cardiomyocytes + Fibroblasts | 0.12 | 0.8 | Intergenic | LINC00290 | U |
| 6994 | chr1 | 209815883 | 209815941 | Heart Cardiomyocytes + Fibroblasts | 0.22 | 0.89 | Intron | LAMB3 | U |
| 6995 | chr5 | 1121519 | 1121593 | Heart Cardiomyocytes + Fibroblasts | 0.22 | 0.89 | Intergenic | SLC12A7 | U |
| 6996 | chr2 | 202874350 | 202874621 | Heart Cardiomyocytes + Fibroblasts | 0.2 | 0.87 | Intergenic | FZD7 | U |
| 6997 | chr2 | 108429756 | 108430094 | Heart Cardiomyocytes + Fibroblasts | 0.18 | 0.85 | Intergenic | LOC729121 | U |
| 6998 | chr15 | 76052866 | 760533541 | Heart Cardiomyocytes + Fibroblasts | 0.2 | 0.87 | Intergenic, TTS | MIR4313, MIR4313 | U |
| 6999 | chr15 | 100472896 | 100472939 | Heart Cardiomyocytes + Fibroblasts | 0.22 | 0.88 | Intergenic | DNM1P46 | U |
| 7000 | chr10 | 88973639 | 88973790 | Heart Cardiomyocytes + Fibroblasts | 0.26 | 0.92 | Intergenic | FAM22A | U |
| 7001 | chr1 | 2285565273 | 228556588 | Heart Cardiomyocytes + Fibroblasts | 0.24 | 0.89 | exon | OBSCN | U |
| 7002 | chr1 | 13861838 | 13862135 | Heart Cardiomyocytes + Fibroblasts | 0.18 | 0.83 | Intergenic | LRRC38 | U |
| 7003 | chr3 | 194880273 | 194880583 | Heart Cardiomyocytes + Fibroblasts | 0.24 | 0.89 | intron | XXYLTJ | U |
| 7004 | chr7 | 18095378 | 18095706 | Heart Cardiomyocytes + Fibroblasts | 0.22 | 0.87 | Intergenic | PRPS1LI | U |
| 7005 | chr1 | 9643254 | 9643716 | Heart Cardiomyocytes + Fibroblasts | 0.26 | 0.91 | TTS | SLC25A33 | U |
| 7006 | chr10 | 10954582 | 10954771 | Heart Cardiomyocytes + Fibroblasts | 0.27 | 0.91 | Intergenic | LINC00710 | U |
| 7007 | chr4 | 184747561 | 184747845 | Heart Cardiomyocytes + Fibroblasts | 0.22 | 0.86 | Intergenic | STOX2 | U |
| 7008 | chr17 | 413761 | 414081 | Heart Cardiomyocytes + Fibroblasts | 0.18 | 0.82 | exon | VPS53 | U |
| 7009 | chr5 | 11012629 | 11012972 | Heart Cardiomyocytes + Fibroblasts | 0.21 | 0.85 | intron | CTNND2 | U |
| 7010 | chr20 | 56644429 | 56644576 | Heart Cardiomyocytes + Fibroblasts | 0.24 | 0.87 | Intergenic | C20orf85 | U |
| 7011 | chr10 | 1198278 | 1198475 | Heart Cardiomyocytes + Fibroblasts | 0.26 | 0.89 | Intergenic | LINC00200 | U |
| 7012 | chr1 | 303938083 | 30394055 | Heart Cardiomyocytes + Fibroblasts | 0.12 | 0.75 | Intergenic | MATN1-AS1 | U |
| 7013 | chr16 | 84092508 | 84092811 | Heart Cardiomyocytes + Fibroblasts | 0.26 | 0.89 | intron | MBTPS1 | U |
| 7014 | chr1 | 3706705 | 3706858 | Heart Cardiomyocytes + Fibroblasts | 0.28 | 0.9 | intron | LRRC47 | U |
| 7015 | chr19 | 5876222 | 5876396 | Heart Cardiomyocytes + Fibroblasts | 0.22 | 0.84 | Intergenic | FUT5 | U |
| 7016 | chr21 | 38966582 | 38966781 | Heart Cardiomyocytes + Fibroblasts | 0.19 | 0.81 | Intergenic | DYRK1A | U |
| 7017 | chr9 | 129946857 | 129947147 | Heart Cardiomyocytes + Fibroblasts | 0.26 | 0.88 | intron | RALGPS1 | U |
| 7018 | chr5 | 180207057 | 180207415 | Heart Cardiomyocytes + Fibroblasts | 0.18 | 0.8 | Intergenic | MGAT1 | U |
| 7019 | chr5 | 56412004 | 56412368 | Heart Cardiomyocytes + Fibroblasts | 0.3 | 0.92 | Intergenic | GPBP1 | U |
| 7020 | chr11 | 125551773 | 125552244 | Heart Cardiomyocytes + Fibroblasts | 0.3 | 0.92 | promoter-TSS,Interg [symbol] | ACRV1, ACRV1 | U |
| 7021 | chr17 | 12999196 | 12999266 | Heart Cardiomyocytes + Fibroblasts | 0.26 | 0.87 | Intergenic | ELAC2 | U |
| 7022 | chr13 | 36448833 | 36448912 | Heart Cardiomyocytes + Fibroblasts | 0.25 | 0.86 | intron | DCLK1 | U |
| 7023 | chr1 | 20634795 | 20635032 | Heart Cardiomyocytes + Fibroblasts | 0.24 | 0.85 | intron | VWA5B1 | U |
| 7024 | chr8 | 94029501 | 94029905 | Heart Cardiomyocytes + Fibroblasts | 0.2 | 0.81 | Intergenic | TRIQK | U |
| 7025 | chr2 | 10902655 | 10902720 | Heart Cardiomyocytes + Fibroblasts | 0.27 | 0.87 | intron | ATP6V1C2 | U |
| 7026 | chr2 | 235080572 | 235080772 | Heart Cardiomyocytes + Fibroblasts | 0.31 | 0.91 | Intergenic | SPP2 | U |
| 7027 | chr18 | 12997061 | 12997279 | Heart Cardiomyocytes + Fibroblasts | 0.22 | 0.82 | intron | CEP192 | U |
| 7028 | chr15 | 45232242 | 45232568 | Heart Cardiomyocytes + Fibroblasts | 0.28 | 0.88 | Intergenic | C15orf43 | U |
| 7029 | chr5 | 123964522 | 123964654 | Heart Cardiomyocytes + Fibroblasts | 0.26 | 0.85 | Intergenic | ZNF608 | U |
| 7030 | chr10 | 125971114 | 125971369 | Heart Cardiomyocytes + Fibroblasts | 0.27 | 0.86 | Intergenic | CHST15 | U |
| 7031 | chr7 | 3272911 | 3273316 | Heart Cardiomyocytes + Fibroblasts | 0.27 | 0.86 | Intergenic | SDK1 | U |
| 7032 | chr7 | 91361379 | 91361797 | Heart Cardiomyocytes + Fibroblasts | 0.22 | 0.81 | Intergenic | MTERF | U |
| 7033 | chr8 | 10377668 | 10377892 | Heart Cardiomyocytes + Fibroblasts | 0.29 | 0.87 | Intergenic | PRSS55 | U |
| 7034 | chr12 | 121009925 | 121010027 | Heart Cardiomyocytes + Fibroblasts | 0.32 | 0.88 | intron | RNF10 | U |
| 7035 | chr12 | 52936552 | 52936739 | Heart Cardiomyocytes + Fibroblasts | 0.34 | 0.9 | Intergenic | KRT71 | U |
| 7036 | chr7 | 141088827 | 141089077 | Heart Cardiomyocytes + Fibroblasts | 0.3 | 0.85 | intron | TMEM178B | U |
| 7037 | chr14 | 100566571 | 100566935 | Heart Cardiomyocytes + Fibroblasts | 0.36 | 0.91 | intron | EVL | U |
| 7038 | chr2 | 161231473 | 161231850 | Heart Cardiomyocytes + Fibroblasts | 0.31 | 0.86 | intron | RBMS1 | U |
| 7039 | chr8 | 53475277 | 53475531 | Heart Cardiomyocytes + Fibroblasts | 0.26 | 0.8 | intron | FAM150A | U |
| 7040 | chr2 | 62283634 | 62283937 | Heart Cardiomyocytes + Fibroblasts | 0.32 | 0.86 | intron | COMMD1 | U |
| 7041 | chr10 | 129481743 | 129482151 | Heart Cardiomyocytes + Fibroblasts | 0.28 | 0.82 | Intergenic | FOXI2 | U |
| 7042 | chr3 | 13289021 | 13289445 | Heart Cardiomyocytes + Fibroblasts | 0.31 | 0.85 | Intergenic | NUP210 | U |
| 7043 | chr15 | 1006537901 | 100653967 | Heart Cardiomyocytes + Fibroblasts | 0.34 | 0.87 | intron | ADAMTS17 | U |
| 7044 | chr8 | 26084698 | 26085094 | Heart Cardiomyocytes + Fibroblasts | 0.3 | 0.82 | Intergenic | PPP2R2A | U |
| 7045 | chr4 | 143002457 | 143002854 | Heart Cardiomyocytes + Fibroblasts | 0.34 | 0.86 | intron | INPP4B | U |
| 7046 | chr1 | 165378811 | 165379230 | Heart Cardiomyocytes + Fibroblasts | 0.14 | 0.91 | intron | RXRG | U |
| 7047 | chr8 | 53788177 | 53788274 | Heart Cardiomyocytes + Fibroblasts | 0.16 | 0.86 | Intergenic | NPBWR1 | U |
| 7048 | chr8 | 25435658 | 25435851 | Heart Cardiomyocytes + Fibroblasts | 0.24 | 0.87 | Intergenic | CDCA2 | U |
| 7049 | chr13 | 38521846 | 38522176 | Heart Cardiomyocytes + Fibroblasts | 0.25 | 0.88 | Intergenic | TRPC4 | U |
| 7050 | chr8 | 140847731 | 140848082 | Heart Cardiomyocytes + Fibroblasts | 0.06 | 0.91 | intron | TRAPPC9 | U |
| 7051 | chr1 | 154680603 | 154680768 | Heart Cardiomyocytes + Fibroblasts | 0.11 | 0.91 | exon | KCNN3 | U |
| 7052 | chr6 | 11999385 | 11999805 | Heart Cardiomyocytes + Fibroblasts | 0.11 | 0.91 | Intergenic | HIVEP1 | U |
| 7053 | chr22 | 17620937 | 17621309 | Heart Cardiomyocytes + Fibroblasts | 0.11 | 0.86 | intron | CECRS | U |
| 7054 | chr1 | 154680517 | 154680769 | Heart Cardiomyocytes + Fibroblasts | 0.12 | 0.89 | exon | KCNNS | U |
| 7055 | chr14 | 57418432 | 57418535 | Heart Cardiomyocytes + Fibroblasts | 0.1 | 0.85 | Intergenic | OTX2-AS | U |
| 7056 | chr14 | 71650466 | 71650630 | Heart Cardiomyocytes + Fibroblasts | 0.18 | 0.88 | Intergenic | SNORD56B | U |
| 7057 | chr6 | 7425578 | 7425652 | Heart Cardiomyocytes + Fibroblasts | 0.23 | 0.91 | Intergenic | RIOK1 | U |
| 7058 | chr20 | 25445183 | 25445374 | Heart Cardiomyocytes + Fibroblasts | 0.18 | 0.86 | intron | NINI | U |
| 7059 | chr1 | 243432142 | 243432313 | Heart Cardiomyocytes + Fibroblasts | 0.21 | 0.88 | intron | SDCCAG8 | U |
| 7060 | chr9 | 90424981 | 90425337 | Heart Cardiomyocytes + Fibroblasts | 0.2 | 0.86 | Intergenic | CTSL1P8 | U |
| 7061 | chr9 | 98978744 | 98979120 | Heart Cardiomyocytes + Fibroblasts | 0.26 | 0.91 | Intergenic | HSD1783 | U |
| 7062 | chr9 | 99842251 | 99842674 | Heart Cardiomyocytes + Fibroblasts | 0.22 | 0.85 | intron | LOC340508 | U |
| 7063 | chr11 | 134232343 | 134232613 | Heart Cardiomyocytes + Fibroblasts | 0.26 | 0.87 | intron | GLB1L2 | U |
| 7064 | chr9 | 89251759 | 89251953 | Heart Cardiomyocytes + Fibroblasts | 0.2 | 0.79 | Intergenic | ZCCHC6 | U |
| 7065 | chr9 | 98836406 | 98836569 | Heart Cardiomyocytes + Fibroblasts | 0.26 | 0.84 | intron | LOC158435 | U |
| 7066 | chr5 | 122430084 | 122430236 | Heart Cardiomyocytes + Fibroblasts | 0.86 | 0.23 | intron | PRDM6 | M |
| 7067 | chr3 | 138892681 | 138892934 | Heart Cardiomyocytes + Fibroblasts | 0.66 | 0.08 | Intergenic | PISRT1 | M |
| 7068 | chr17 | 6555695 | 6555783 | Heart Cardiomyocytes + Fibroblasts | 0.67 | 0.1 | promoter-TSS | MED31 | M |
| 7069 | chr2 | 2051800041 | 205180213 | Heart Cardiomyocytes + Fibroblasts | 0.71 | 0.15 | Intergenic | PARD3B | M |
| 7070 | chr5 | 122429499 | 122429732 | Heart Cardiomyocytes + Fibroblasts | 0.76 | 0.22 | Intron | PRDM6 | M |
| 7071 | chr4 | 175133405 | 175133826 | Heart Cardiomyocytes + Fibroblasts | 0.76 | 0.22 | Intergenic | CEP44 | M |
| 7072 | chr16 | 88124298 | 88124437 | Heart Cardiomyocytes + Fibroblasts | 0.72 | 0.21 | Intergenic | BANP | M |
| 7073 | chr9 | 139636813 | 139637044 | Heart Cardiomyocytes + Fibroblasts | 0.64 | 0.16 | intron | CN10 | M |
| 7074 | chr2 | 120436418 | 120436491 | Heart Cardiomyocytes + Fibroblasts | 0.64 | 0.17 | promoter-TSS | TMEM177 | M |
| 7075 | chr5 | 122428966 | 122429450 | Heart Cardiomyocytes + Fibroblasts | 0.65 | 0.18 | intron | PRDM6 | M |
| 7076 | chr15 | 63674449 | 63674484 | Heart Cardiomyocytes + Fibroblasts | 0.62 | 0.16 | promoter-TSS | CA12 | M |
| 7077 | chr5 | 32843436 | 32843583 | Heart Cardiomyocytes + Fibroblasts | 0.59 | 0.15 | Intergenic | LOC340113 | M |
| 7078 | chr11 | 67417821 | 67417974 | Heart Cardiomyocytes + Fibroblasts | 0.66 | 0.25 | intron | ACY3 | M |
| 7079 | chr22 | 51059793 | 51060033 | Heart Cardiomyocytes + Fibroblasts | 0.61 | 0.21 | Intergenic | ARSA | M |
| 7080 | chr14 | 957576751 | 95757926 | Heart Cardiomyocytes + Fibroblasts | 0.63 | 0.23 | intron | CLMN | M |
| 7081 | chr1 | 244504815 | 244505172 | Heart Cardiomyocytes + Fibroblasts | 0.55 | 0.22 | Intergenic | C1orf100 | M |
| 7082 | chr19 | 51069087 | 51069248 | Heart Cardiomyocytes + Fibroblasts | 0.61 | 0.29 | intron | LRRC4B | M |
| 7083 | chr5 | 1224298551 | 122430020 | Heart Cardiomyocytes + Fibroblasts | 0.84 | 0.23 | intron | PRDM6 | M |
| 7084 | chr5 | 1224303261 | 122430510 | Heart Cardiomyocytes + Fibroblasts | 0.86 | 0.26 | intron | PRDM6 | M |
| 7085 | chr5 | 122422553 | 122422913 | Heart Cardiomyocytes + Fibroblasts | 0.7 | 0.19 | Intergenic | PRDM6 | M |
| 7086 | chr5 | 1224343793 | 122434629 | Heart Cardiomyocytes + Fibroblasts | 0.66 | 0.18 | intron | PRDM6 | M |
| 7087 | chr4 | 175138256 | 175138325 | Heart Cardiomyocytes + Fibroblasts | 0.62 | 0.19 | Intergenic | CEP44 | M |
| 7088 | chr15 | 92938493 | 92938521 | Heart Cardiomyocytes + Fibroblasts | 0.57 | 0.17 | intron | ST8SIA2 | M |
| 7089 | chr22 | 39152650 | 39152783 | Heart Cardiomyocytes + Fibroblasts | 0.58 | 0.19 | promoter-TSS | SUN2 | M |
| 7090 | chr5 | 122434244 | 122434305 | Heart Cardiomyocytes + Fibroblasts | 0.6 | 0.22 | intron | PRDMS | M |

[symbol]

indicates data missing or illegible when filed

TABLE C
Example Sequence and A nnotations in Sequence Listing

<210> 2
<211> 283
<212> DMA
<213> Homo sapiens
<220
<221> source
<222> (1) . . . (283)
<223> chr9:119238427-119238709; Gene: ASTN2 (intron)
<220>
<221> misc_feature
<222> (1) . . . (283)
<223> U/M: U (0.05:0.94) in Oral, Larynx and Esophageal epithelium
<400> 2

| | |
|---|---|
| cggatggccc gaggtgttcc ttggcttgag geggcatcac tctaatctca gcctctgtct | 60 |
| tcacatgcct tctttgctct gtcttctcag agcggcatca gtcactgaat taaggaatta | 120 |
| acttaatcca gtattacctc atcttgatcc ttaagtaaat acatctgcaa agatgatttc | 180 |
| caaataaagc cccatcccaa ggtttcaggt agatgtgaat ttttcaagga cactgttcaa | 240 |
| cccactgcaa tccatcctct cacttcctca gaagacttgc agc | 283 |

In accordance with one embodiment of the present disclosure, a method is provided for identifying that a biological sample includes DNA from an oral, larynx or esophageal epithelial cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 1-15 or 16-90. In some embodiments, the method then identifies the target DNA fragment as being from an oral, larynx or esophageal epithelial cell when no more than 40% of the CpG sites are methylated. In some embodiments, the method identifies the target DNA fragment as being from an oral, larynx or esophageal epithelial cell when no more than 25%, 30%, 35%, 40%, 45%, or 50% of the CpG sites are methylated. Likewise, in some embodiments, the method identifies the target DNA fragment as being from an oral, larynx or esophageal epithelial cell when at least 50%, 55%, 60%, 65%, 70%, 75%, 80%, 85% or 90% of the CpG sites are unmethylated. In some embodiments, the method identifies the target DNA fragment as not being from an oral, larynx or esophageal epithelial cell when at least 50%, 55%, 60%, 65%, 70%, 75%, 80%, 85% or 90% of the CpG sites are methylated. In some embodiments, the method identifies the target DNA fragment as not being from an oral, larynx or esophageal epithelial cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 126-133. In some embodiments, the method then identifies the target DNA fragment as being from an oral, larynx or esophageal epithelial cell when 50% or more of the CpG sites are methylated. In some embodiments, the method identifies the target DNA fragment as being from an oral, larynx or esophageal epithelial cell when at least 55%, 60%, 65%, 70%, 75%, 80%, 85% or 90% of the CpG sites are methylated. Likewise, in some embodiments, the method identifies the target DNA fragment as being from an oral, larynx or esophageal epithelial cell when no more than 25%, 30%, 35%, 40%, 45%, or 50% of the CpG sites are unmethylated. In some embodiments, the method identifies the target DNA fragment as not being from an oral, larynx or esophageal epithelial cell when no more than 25%, 30%, 35%, 40%16, 45%, or 50% of the CpG sites are methylated. In some embodiments, the method identifies the target DNA fragment as not being from an oral, larynx or esophageal epithelial cell when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 1-15, 16-90, 91-91, 92-101, 102-125, 126-133, 134-134 or 135-150.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 1-15, 16-90, 91-91, 92-101 or 102-125. In some embodiments, the method then identifies the target DNA fragment as being from an oral, larynx or esophageal epithelial cell when no more than 40% of the CpG sites are methylated. In some embodiments, the method identifies the target DNA fragment as being from an oral, larynx or esophageal epithelial cell when no more than 25%, 30%, 35%, 40%, 45%, or 50% of the CpG sites are methylated. Likewise, in some embodiments, the method identifies the target DNA fragment as being from an oral, larynx or esophageal epithelial cell when at least 50%, 55%, 60%, 65%, 70%, 75%, 80%, 85% or 90% of the CpG sites are unmethylated. In some embodiments, the method identifies the target DNA fragment as not being from an oral, larynx or esophageal epithelial cell when at least 50%, 55%, 60%, 65%, 70%, 75%, 80%, 85% or 90% of the CpG sites are methylated. In some embodiments, the method identifies the target DNA fragment as not being from an oral, larynx or esophageal epithelial cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 126-133, 134-134 or 135-150. In some embodiments, the method then identifies the target DNA fragment as being from an oral, larynx or esophageal epithelial cell when 50% or more of the CpG sites are methylated. In some embodiments, the method identifies the target DNA fragment as being from an oral, larynx or esophageal epithelial cell when at least 55%, 60%, 65%, 70%, 75%, 80%, 85% or 90% of the CpG sites are methylated. Likewise, in some embodiments, the method identifies the target DNA fragment as being from an oral, larynx or esophageal epithelial cell when no more than 25%, 30%, 35%, 40%, 45%, or 50% of the CpG sites are unmethylated. In some embodiments, the method identifies the target DNA fragment as not being from an oral, larynx or esophageal epithelial cell when no more than 25%, 30%, 35%, 40%, 45%, or 50% of the CpG sites are methylated. In some embodiments, the method identifies the target DNA fragment as not being from an oral, larynx or esophageal epithelial cell when at least 50%, 55%, 60%, 65%, 70%, 75%, 80%, 85% or 90% of the CpG sites are unmethylated.

In some embodiments, when the prediction from two or more of the above methods agrees with another, the prediction result is further affirmed.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from an oral, larynx or esophageal epithelial cell of a subject, the method indicates that the subject has abnormal cell death and/or a disease relating to the cell. In some embodiments, the disease or condition is injury, inflammation, or cancer of the oral, larynx or esophageal epithelium.

In some embodiments, when the amount of cell-free DNA identified as being from a particular type of cell or cells, e.g., oral, larynx or esophageal epithelial cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., oral, larynx or esophageal epithelial cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as an oral, larynx or esophageal epithelial cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon.

Once the primary origin of the cancer is identified, the subject may be treated with appropriate regiments for that cancer type.

### A2. Gastric Epithelium

Also as provided in Table A, some genomic locations are uniformly under-methylated or over-methylated in gastric epithelial cells as compared to all other cell types in the human.

In accordance with one embodiment of the present disclosure, a method is provided for identifying that a biological sample includes DNA from a gastric epithelial cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 151-170, 171-330, 331-335, 336-340 or 341-378, or selected from SEQ ID NO: 151-170 or 171-330. In some embodiments, the method then identifies the target DNA fragment as being from a gastric epithelial cell when no more than 40% of the CpG sites are methylated. In some embodiments, the method identifies the target DNA fragment as being from a gastric epithelial cell when no more than 25%, 30%, 35%, 40%, 45%, or 50% of the CpG sites are methylated. Likewise, in some embodiments, the method identifies the target DNA fragment as being from a gastric epithelial cell when at least 50%, 55%, 60%, 65%, 70%, 75%, 80%, 85% or 90% of the CpG sites are unmethylated. In some embodiments, the method identifies the target DNA fragment as not being from a gastric epithelial cell when at least 50%, 55%, 60%, 65%, 70%, 75%, 80%, 85% or 90% of the CpG sites are methylated. In some embodiments, the method identifies the target DNA fragment as not being from a gastric epithelial cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 379-401, 402-402 or 403-428, or selected from SEQ ID NO: 379-401. In some embodiments, the method then identifies the target DNA fragment as being from a gastric epithelial cell when 50% or more of the CpG sites are methylated. In some embodiments, the method identifies the target DNA fragment as being from a gastric epithelial cell when at least 55%, 60%, 65%, 70%, 75%, 80%, 85% or 90% of the CpG sites are methylated. Likewise, in some embodiments, the method identifies the target DNA fragment as being from a gastric epithelial cell when no more than 25%, 30%, 35%, 40%, 45%, or 50% of the CpG sites are unmethylated. In some embodiments, the method identifies the target DNA fragment as not being from a gastric epithelial cell when no more than 25%, 30%, 35%, 40%, 45%, or 50% of the CpG sites are methylated. In some embodiments, the method identifies the target DNA fragment as not being from a gastric epithelial cell when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 151-170, 171-330, 331-335, 336-340, 341-378, 379-401, 402-402 or 403-428.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a gastric epithelial cell of a subject, the method indicates that the subject has abnormal cell death and/or a disease relating to the cell. In some embodiments, the disease or condition is injury, inflammation, or cancer of the gastric epithelium.

In some embodiments, when the amount of cell-free DNA identified as being from a particular type of cell or cells, e.g., gastric epithelial cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., gastric epithelial cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a gastric epithelial cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### A3. Small Intestine Epithelium

Also as provided in Table A, some genomic locations are uniformly under-methylated or over-methylated in small intestine epithelial cells as compared to all other cell types in the human.

In accordance with one embodiment of the present disclosure, a method is provided for identifying that a biological sample includes DNA from a small intestine epithelial cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 429-446, 447-527, 528-529, 530-536 or 537-554, or selected from SEQ ID NO: 429-446 or 447-527. In some embodiments, the method then identifies the target DNA fragment as being from a small intestine epithelial cell when no more than 40% of the CpG sites are methylated. In some embodiments, the method identifies the target DNA fragment as being from a small intestine epithelial cell when no more than 25%, 30%, 35%, 40%, 45%, or 50% of the CpG sites are methylated. Likewise, in some embodiments, the method identifies the target DNA fragment as being from a small intestine epithelial cell when at least 50%, 55%, 60%, 65%, 70%, 75%, 80%, 85% or 90% of the CpG sites are unmethylated. In some embodiments, the method identifies the target DNA fragment as not being from a small intestine epithelial cell when at least 50%, 55%, 60%, 65%, 70%, 75%, 80%, 85% or 90% of the CpG sites are methylated. In some embodiments, the method identifies the target DNA fragment as not being from a small intestine epithelial cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 555-564, 565-565 or 566-579, or selected from SEQ ID NO: 555-564. In some embodiments, the method then identifies the target DNA fragment as being from a small intestine epithelial cell when 50% or more of the CpG sites are methylated. In some embodiments, the method identifies the target DNA fragment as being from a small intestine epithelial cell when at least 55%, 60%, 65%, 70%, 75%, 80%, 85% or 90% of the CpG sites are methylated. Likewise, in some embodiments, the method identifies the target DNA fragment as being from a small intestine epithelial cell when no more than 25%, 30%, 16, 35%, 40%, 45%, or 50%, 16 of the CpG sites are unmethylated. In some embodiments, the method identifies the target DNA fragment as not being from a small intestine epithelial cell when no more than 25%, 30%, 35%, 40%, 45%, or 50% of the CpG sites are methylated. In some embodiments, the method identifies the target DNA fragment as not being from a small intestine epithelial cell when at least 50% i, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 429-446, 447-527, 528-529, 530-536, 537-554, 555-564, 565-565 or 566-579.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a small intestine epithelial cell of a subject, the method indicates that the subject has abnormal cell death and/or a disease relating to the cell. In some embodiments, the disease or condition is injury, inflammation, or cancer of the small intestine epithelium.

In some embodiments, when the amount of cell-free DNA identified as being from a particular type of cell or cells, e.g., small intestine epithelial cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., small intestine epithelial cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a small intestine epithelial cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### A4. Colon Epithelium

Also as provided in Table A, some genomic locations are uniformly under-methylated or over-methylated in colon epithelial cells as compared to all other cell types in the human.

In accordance with one embodiment of the present disclosure, a method is provided for identifying that a biological sample includes DNA from a colon epithelial cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 580-596, 597-657, 658-660, 661-668 or 669-704, or selected from SEQ ID NO: 580-596 or 597-657. In some embodiments, the method then identifies the target DNA fragment as being from a colon epithelial cell when no more than 40% of the CpG sites are methylated. In some embodiments, the method identifies the target DNA fragment as being from a colon epithelial cell when no more than 25%, 30%, 35%, 40%, 45%, or 50% of the CpG sites are methylated. Likewise, in some embodiments, the method identifies the target DNA fragment as being from a colon epithelial cell when at least 50%, 55%, 60%, 65%, 70%, 75%, 80%, 85% or 90% of the CpG sites are unmethylated. In some embodiments, the method identifies the target DNA fragment as not being from a colon epithelial cell when at least 50%, 55%, 60%, 65%, 70%, 75%, 80%, 85% or 90% of the CpG sites are methylated. In some embodiments, the method identifies the target DNA fragment as not being from a colon epithelial cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 705-715 or 716-729, or selected from SEQ ID NO: 705-715. In some embodiments, the method then identifies the target DNA fragment as being from a colon epithelial cell when 50% or more of the CpG sites are methylated. In some embodiments, the method identifies the target DNA fragment as being from a colon epithelial cell when at least 55%, 60%, 65%, 70%, 75%, 80%, 85% or 90% of the CpG sites are methylated. Likewise, in some embodiments, the method identifies the target DNA fragment as being from a colon epithelial cell when no more than 25%, 30%, 35%, 40%, 45%, or 50% of the CpG sites are unmethylated. In some embodiments, the method identifies the target DNA fragment as not being from a colon epithelial cell when no more than 25%, 30%, 35%, 40%, 45%, or 50% of the CpG sites are methylated. In some embodiments, the method identifies the target DNA fragment as not being from a colon epithelial cell when at least 50%, 55%, 60%, 65%, 70%, 75%, 80%, 85% or 90/a of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 580-596, 597-657, 658-660, 661-668, 669-704, 705-715 or 716-729.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a colon epithelial cell of a subject, the method indicates that the subject has abnormal cell death and/or a disease relating to the cell. In some embodiments, the disease or condition is injury, inflammation, or cancer of the colon epithelium.

In some embodiments, when the amount of cell-free DNA identified as being from a particular type of cell or cells, e.g., colon epithelial cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., colon epithelial cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a colon epithelial cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### A5. Colon Fibroblasts

Also as provided in Table A, some genomic locations are uniformly under-methylated or over-methylated in colon fibroblast cells as compared to all other cell types in the human.

In accordance with one embodiment of the present disclosure, a method is provided for identifying that a biological sample includes DNA from a colon fibroblast cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 730-732. In some embodiments, the method then identifies the target DNA fragment as being from a colon fibroblast cell when no more than 40% of the CpG sites are methylated. In some embodiments, the method identifies the target DNA fragment as being from a colon fibroblast cell when no more than 25%, 30%, 35%, 40%, 45%, or 50% of the CpG sites are methylated. Likewise, in some embodiments, the method identifies the target DNA fragment as being from a colon fibroblast cell when at least 50%, 55%, 60%, 65%, 70%, 75%, 80%, 85% or 90% of the CpG sites are unmethylated. In some embodiments, the method identifies the target DNA fragment as not being from a colon fibroblast cell when at least 50%, 55%, 60%, 65%, 70%, 75%, 80%, 85% or 90% of the CpG sites are methylated. In some embodiments, the method identifies the target DNA fragment as not being from a colon fibroblast cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 733-739 or 740-741, or selected from SEQ ID NO: 733-739. In some embodiments, the method then identifies the target DNA fragment as being from a colon fibroblast cell when 50% or more of the CpG sites are methylated. In some embodiments, the method identifies the target DNA fragment as being from a colon fibroblast cell when at least 55%, 60%, 65%, 70%, 75%, 80%, 85% or 90% of the CpG sites are methylated. Likewise, in some embodiments, the method identifies the target DNA fragment as being from a colon fibroblast cell when no more than 25%, 30%, 35%, 40%, 45%, or 50% of the CpG sites are unmethylated. In some embodiments, the method identifies the target DNA fragment as not being from a colon fibroblast cell when no more than 25%, 30%, 35%, 40%, 45%, or 50% of the CpG sites are methylated. In some embodiments, the method identifies the target DNA fragment as not being from a colon fibroblast cell when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 730-732, 733-739 or 740-741.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a colon fibroblast cell of a subject, the method indicates that the subject has abnormal cell death and/or a disease relating to the cell. In some embodiments, the disease or condition is injury, inflammation, or cancer of the colon fibroblast.

In some embodiments, when the amount of cell-free DNA identified as being from a particular type of cell or cells, e.g., colon fibroblast cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., colon fibroblast cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a colon fibroblast cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### A6. Gallbladder Epithelium

Also as provided in Table A, some genomic locations are uniformly under-methylated or over-methylated in gallbladder epithelial cells as compared to all other cell types in the human.

In accordance with one embodiment of the present disclosure, a method is provided for identifying that a biological sample includes DNA from a gallbladder epithelial cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 742-758, 759-829, 830-831, 832-839 or 840-867, or selected from SEQ ID NO: 742-758 or 759-829. In some embodiments, the method then identifies the target DNA fragment as being from a gallbladder epithelial cell when no more than 40% of the CpG sites are methylated. In some embodiments, the method identifies the target DNA fragment as being from a gallbladder epithelial cell when no more than 25%, 30%, 35%, 40%, 45%, or 50% of the CpG sites are methylated. Likewise, in some embodiments, the method identifies the target DNA fragment as being from a gallbladder epithelial cell when at least 50%, 55%, 60%, 65%, 70%, 75%, 80%, 85% or 90% of the CpG sites are unmethylated. In some embodiments, the method identifies the target DNA fragment as not being from a gallbladder epithelial cell when at least 50%, 55%, 60%, 65%, 70%, 75%, 80%, 85% or 90% of the CpG sites are methylated. In some embodiments, the method identifies the target DNA fragment as not being from a gallbladder epithelial cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 868-875 or 876-876, or selected from SEQ ID NO: 868-875. In some embodiments, the method then identifies the target DNA fragment as being from a gallbladder epithelial cell when 50% or more of the CpG sites are methylated. In some embodiments, the method identifies the target DNA fragment as being from a gallbladder epithelial cell when at least 55%, 60%, 65%, 70%, 75%, 80%, 85% or 90% of the CpG sites are methylated. Likewise, in some embodiments, the method identifies the target DNA fragment as being from a gallbladder epithelial cell when no more than 25%, 30%, 35%, 40%, 45%, or 50% of the CpG sites are unmethylated. In some embodiments, the method identifies the target DNA fragment as not being from a gallbladder epithelial cell when no more than 25%, 30%, 35%, 40%, 45%, or 50%, 60% of the CpG sites are methylated. In some embodiments, the method identifies the target DNA fragment as not being from a gallbladder epithelial cell when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 742-758, 759-829, 830-831, 832-839, 840-867, 868-875 or 876-876.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a gallbladder epithelial cell of a subject, the method indicates that the subject has abnormal cell death and/or a disease relating to the cell. In some embodiments, the disease or condition is injury, inflammation, or cancer of the gallbladder epithelium.

In some embodiments, when the amount of cell-free DNA identified as being from a particular type of cell or cells, e.g., gallbladder epithelial cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., gallbladder epithelial cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a gallbladder epithelial cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### A7. Liver Hepatocytes

Also as provided in Table A, some genomic locations are uniformly under-methylated or over-methylated in liver hepatocytes as compared to all other cell types in the human.

In accordance with one embodiment of the present disclosure, a method is provided for identifying that a biological sample includes DNA from a liver hepatocyte. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 877-896, 897-980, 981-983, 984-986, 987-988 or 989-1002, or selected from SEQ ID NO: 877-896 or 897-980. In some embodiments, the method then identifies the target DNA fragment as being from a liver hepatocyte when no more than 40% of the CpG sites are methylated. In some embodiments, the method identifies the target DNA fragment as being from a liver hepatocyte when no more than 25%, 30%, 35%, 40%, 45%, or 50% of the CpG sites are methylated. Likewise, in some embodiments, the method identifies the target DNA fragment as being from a liver hepatocyte when at least 50%, 55%, 60%, 65%, 70%, 75%, 80%, 85% or 90% of the CpG sites are unmethylated. In some embodiments, the method identifies the target DNA fragment as not being from a liver hepatocyte when at least 50%, 55%, 60%, 65%, 70%, 75%, 80%, 85% or 90% of the CpG sites are methylated. In some embodiments, the method identifies the target DNA fragment as not being from a liver hepatocyte when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 1003-1018, 1019-1023 or 1024-1027, or selected from SEQ ID NO: 1003-1018. In some embodiments, the method then identifies the target DNA fragment as being from a liver hepatocyte when 50% or more of the CpG sites are methylated. In some embodiments, the method identifies the target DNA fragment as being from a liver hepatocyte when at least 55%, 60%, 65%, 70%, 75%, 80%, 85% or 90% of the CpG sites are methylated. Likewise, in some embodiments, the method identifies the target DNA fragment as being from a liver hepatocyte when no more than 25%, 30%, 35%, 40%, 45%, or 50% of the CpG sites are unmethylated. In some embodiments, the method identifies the target DNA fragment as not being from a liver hepatocyte when no more than 25%, 30%, 35%, 40%, 45%, or 50% of the CpG sites are methylated. In some embodiments, the method identifies the target DNA fragment as not being from a liver hepatocyte when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 877-896, 897-980, 981-983, 984-986, 987-988, 989-1002, 1003-1018, 1019-1023 or 1024-1027.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a liver hepatocyte of a subject, the method indicates that the subject has abnormal cell death and/or a disease relating to the cell. In some embodiments, the disease or condition is injury, inflammation, or cancer of the liver hepatocytes.

In some embodiments, when the amount of cell-free DNA identified as being from a particular type of cell or cells, e.g., liver hepatocytes, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., liver hepatocytes is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a liver hepatocyte, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### A8. Pancreatic Acinar Cells

Also as provided in Table A, some genomic locations are uniformly under-methylated or over-methylated in pancreatic acinar cells as compared to all other cell types in the human.

In accordance with one embodiment of the present disclosure, a method is provided for identifying that a biological sample includes DNA from a pancreatic acinar cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 1028-1041,1042-1112, 1113-1116, 1117-1127 or 1128-1155, or selected from SEQ ID NO: 1028-1041 or 1042-1112. In some embodiments, the method then identifies the target DNA fragment as being from a pancreatic acinar cell when no more than 40% of the CpG sites are methylated. In some embodiments, the method identifies the target DNA fragment as being from a pancreatic acinar cell when no more than 25%, 30%, 35%, 40%, 45%, or 50% of the CpG sites are methylated. Likewise, in some embodiments, the method identifies the target DNA fragment as being from a pancreatic acinar cell when at least 50%, 55%, 60%, 65%, 70%, 75%, 80%, 85% or 90% of the CpG sites are unmethylated. In some embodiments, the method identifies the target DNA fragment as not being from a pancreatic acinar cell when at least 50%, 55%, 60%, 65%, 70%, 75%, 80%, 85% or 90% of the CpG sites are methylated. In some embodiments, the method identifies the target DNA fragment as not being from a pancreatic acinar cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 1156-1161 or 1162-1180, or selected from SEQ ID NO: 1156-1161. In some embodiments, the method then identifies the target DNA fragment as being from a pancreatic acinar cell when 50% or more of the CpG sites are methylated. In some embodiments, the method identifies the target DNA fragment as being from a pancreatic acinar cell when at least 55%, 60%, 65%, 70%, 75%, 80%, 85% or 90% of the CpG sites are methylated. Likewise, in some embodiments, the method identifies the target DNA fragment as being from a pancreatic acinar cell when no more than 25%, 30%, 35%, 40%, 45%, or 50% of the CpG sites are unmethylated. In some embodiments, the method identifies the target DNA fragment as not being from a pancreatic acinar cell when no more than 25%, 30%, 35%, 40%, 45%, or 50% of the CpG sites are methylated. In some embodiments, the method identifies the target DNA fragment as not being from a pancreatic acinar cell when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 1028-1041, 1042-1112, 1113-1116, 1117-1127, 1128-1155, 1156-1161 or 1162-1180.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a pancreatic acinar cell of a subject, the method indicates that the subject has abnormal cell death and/or a disease relating to the cell. In some embodiments, the disease or condition is injury, inflammation, or cancer of the pancreatic acinar cells. In some embodiments, the disease is diabetes.

In some embodiments, when the amount of cell-free DNA identified as being from a particular type of cell or cells, e.g., pancreatic acinar cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., pancreatic acinar cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a pancreatic acinar cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### A9. Pancreatic Alpha Cells

Also as provided in Table A, some genomic locations are uniformly under-methylated or over-methylated in pancreatic alpha cells as compared to all other cell types in the human.

In accordance with one embodiment of the present disclosure, a method is provided for identifying that a biological sample includes DNA from a pancreatic alpha cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 1181-1198, 1199-1282, 1283-1284, 1285-1287, 1288-1292 or 1293-1306, or selected from SEQ ID NO: 1181-1198 or 1199-1282. In some embodiments, the method then identifies the target DNA fragment as being from a pancreatic alpha cell when no more than 40% of the CpG sites are methylated. In some embodiments, the method identifies the target DNA fragment as being from a pancreatic alpha cell when no more than 25%, 30%, 35%, 40%, 45%, or 50% of the CpG sites are methylated. Likewise, in some embodiments, the method identifies the target DNA fragment as being from a pancreatic alpha cell when at least 50%, 55%, 60%, 65%, 70%, 75%, 80%, 85% or 90% of the CpG sites are unmethylated. In some embodiments, the method identifies the target DNA fragment as not being from a pancreatic alpha cell when at least 50%, 55%, 60%, 65%, 70%, 75%, 80%, 85% or 90% of the CpG sites are methylated. In some embodiments, the method identifies the target DNA fragment as not being from a pancreatic alpha cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 1307-1315, 1316-1316 or 1317-1331, or selected from SEQ ID NO: 1307-1315. In some embodiments, the method then identifies the target DNA fragment as being from a pancreatic alpha cell when 50% or more of the CpG sites are methylated. In some embodiments, the method identifies the target DNA fragment as being from a pancreatic alpha cell when at least 55%, 60%, 65%, 70%, 75%, 80%, 85% or 90% of the CpG sites are methylated. Likewise, in some embodiments, the method identifies the target DNA fragment as being from a pancreatic alpha cell when no more than 25%, 30%, 35%, 40%, 45%, or 50% of the CpG sites are unmethylated. In some embodiments, the method identifies the target DNA fragment as not being from a pancreatic alpha cell when no more than 25%, 30%, 35%, 40%, 45%, or 50% of the CpG sites are methylated. In some embodiments, the method identifies the target DNA fragment as not being from a pancreatic alpha cell when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 1181-1198, 1199-1282, 1283-1284,1285-1287, 1288-1292,1293-1306, 1307-1315, 1316-1316 or 1317-1331.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a pancreatic alpha cell of a subject, the method indicates that the subject has abnormal cell death and/or a disease relating to the cell. In some embodiments, the disease or condition is injury, inflammation, or cancer of the pancreatic alpha cells. In some embodiments, the disease is diabetes.

In some embodiments, when the amount of cell-free DNA identified as being from a particular type of cell or cells, e.g., pancreatic alpha cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., pancreatic alpha cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a pancreatic alpha cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### A10. Pancreatic Beta Cells

Also as provided in Table A, some genomic locations are uniformly under-methylated or over-methylated in pancreatic beta cells as compared to all other cell types in the human.

In accordance with one embodiment of the present disclosure, a method is provided for identifying that a biological sample includes DNA from a pancreatic beta cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 1332-1351, 1352-1440, 1441-1445 or 1446-1460, or selected from SEQ ID NO: 1332-1351 or 1352-1440. In some embodiments, the method then identifies the target DNA fragment as being from a pancreatic beta cell when no more than 40% of the CpG sites are methylated. In some embodiments, the method identifies the target DNA fragment as being from a pancreatic beta cell when no more than 25%, 30%, 35%, 40%, 45%, or 50% of the CpG sites are methylated. Likewise, in some embodiments, the method identifies the target DNA fragment as being from a pancreatic beta cell when at least 50%, 55%, 60%, 65%, 70%, 75%, 80%, 85% or 90% of the CpG sites are unmethylated. In some embodiments, the method identifies the target DNA fragment as not being from a pancreatic beta cell when at least 50%, 55%, 60%, 65%, 70%, 75%, 80%, 85% or 90% of the CpG sites are methylated. In some embodiments, the method identifies the target DNA fragment as not being from a pancreatic beta cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 1461-1471 or 1472-1485, or selected from SEQ ID NO: 1461-1471. In some embodiments, the method then identifies the target DNA fragment as being from a pancreatic beta cell when 50% or more of the CpG sites are methylated. In some embodiments, the method identifies the target DNA fragment as being from a pancreatic beta cell when at least 55%, 60%, 65%, 70%, 75%, 80%, 85% or 90% of the CpG sites are methylated. Likewise, in some embodiments, the method identifies the target DNA fragment as being from a pancreatic beta cell when no more than 25%, 30%, 35%, 40%, 45%, or 50% of the CpG sites are unmethylated. In some embodiments, the method identifies the target DNA fragment as not being from a pancreatic beta cell when no more than 25%, 30%, 35%, 40%, 45%, or 50% of the CpG sites are methylated. In some embodiments, the method identifies the target DNA fragment as not being from a pancreatic beta cell when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 1332-1351, 1352-1440, 1441-1445, 1446-1460, 1461-1471 or 1472-1485.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a pancreatic beta cell of a subject, the method indicates that the subject has abnormal cell death and/or a disease relating to the cell. In some embodiments, the disease or condition is injury, inflammation, or cancer of the pancreatic beta cells. In some embodiments, the disease is diabetes.

In some embodiments, when the amount of cell-free DNA identified as being from a particular type of cell or cells, e.g., pancreatic beta cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., pancreatic beta cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a pancreatic beta cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### A11. Pancreatic Delta Cells

Also as provided in Table A, some genomic locations are uniformly under-methylated or over-methylated in pancreatic delta cells as compared to all other cell types in the human.

In accordance with one embodiment of the present disclosure, a method is provided for identifying that a biological sample includes DNA from a pancreatic delta cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 1486-1508,1509-1594, 1595-1596, 1597-1598 or 1599-1613, or selected from SEQ ID NO: 1486-1508 or 1509-1594. In some embodiments, the method then identifies the target DNA fragment as being from a pancreatic delta cell when no more than 40% of the CpG sites are methylated. In some embodiments, the method identifies the target DNA fragment as being from a pancreatic delta cell when no more than 25%, 30%, 35%, 40%, 45%, or 50% of the CpG sites are methylated. Likewise, in some embodiments, the method identifies the target DNA fragment as being from a pancreatic delta cell when at least 50%, 55%, 60%, 65%, 70%, 75%, 80%, 85% or 90% of the CpG sites are unmethylated. In some embodiments, the method identifies the target DNA fragment as not being from a pancreatic delta cell when at least 50%, 55%, 60%, 65%, 70%, 75%, 80%, 85% or 90% of the CpG sites are methylated. In some embodiments, the method identifies the target DNA fragment as not being from a pancreatic delta cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 1614-1624, 1625-1625 or 1626-1638, or selected from SEQ ID NO: 1614-1624. In some embodiments, the method then identifies the target DNA fragment as being from a pancreatic delta cell when 50% or more of the CpG sites are methylated. In some embodiments, the method identifies the target DNA fragment as being from a pancreatic delta cell when at least 55%, 60%, 65%, 70%, 75%, 80%, 85% or 90% of the CpG sites are methylated. Likewise, in some embodiments, the method identifies the target DNA fragment as being from a pancreatic delta cell when no more than 25%, 30%, 35%, 40%, 45%, or 50% of the CpG sites are unmethylated. In some embodiments, the method identifies the target DNA fragment as not being from a pancreatic delta cell when no more than 25%, 30%, 35%, 40%, 45%, or 50%, 16 of the CpG sites are methylated. In some embodiments, the method identifies the target DNA fragment as not being from a pancreatic delta cell when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 1486-1508, 1509-1594, 1595-1596, 1597-1598, 1599-1613, 1614-1624, 1625-1625 or 1626-1638.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a pancreatic delta cell of a subject, the method indicates that the subject has abnormal cell death and/or a disease relating to the cell. In some embodiments, the disease or condition is injury, inflammation, or cancer of the pancreatic delta cells. In some embodiments, the disease is diabetes.

In some embodiments, when the amount of cell-free DNA identified as being from a particular type of cell or cells, e.g., pancreatic delta cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., pancreatic delta cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a pancreatic delta cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### A12. Pancreatic Ductal Cells

Also as provided in Table A, some genomic locations are uniformly under-methylated or over-methylated in pancreatic ductal cells as compared to all other cell types in the human.

In accordance with one embodiment of the present disclosure, a method is provided for identifying that a biological sample includes DNA from a pancreatic ductal cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 1639-1658, 1659-1742, 1743-1743, 1744-1747, 1748-1751 or 1752-1767, or selected from SEQ ID NO: 1639-1658 or 1659-1742. In some embodiments, the method then identifies the target DNA fragment as being from a pancreatic ductal cell when no more than 40% of the CpG sites are methylated. In some embodiments, the method identifies the target DNA fragment as being from a pancreatic ductal cell when no more than 25%, 30%, 35%, 40%, 45%, or 50% of the CpG sites are methylated. Likewise, in some embodiments, the method identifies the target DNA fragment as being from a pancreatic ductal cell when at least 50%, 55%, 60%, 65%, 70%, 75%, 80%, 85% or 90% of the CpG sites are unmethylated. In some embodiments, the method identifies the target DNA fragment as not being from a pancreatic ductal cell when at least 50%, 55%, 60%, 65%, 70%, 75%, 80%, 85% or 90% of the CpG sites are methylated. In some embodiments, the method identifies the target DNA fragment as not being from a pancreatic ductal cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 1768-1779 or 1780-1792, or selected from SEQ ID NO: 1768-1779. In some embodiments, the method then identifies the target DNA fragment as being from a pancreatic ductal cell when 50% or more of the CpG sites are methylated. In some embodiments, the method identifies the target DNA fragment as being from a pancreatic ductal cell when at least 55%, 60%, 65%, 70%, 75%, 80%, 85% or 90% of the CpG sites are methylated. Likewise, in some embodiments, the method identifies the target DNA fragment as being from a pancreatic ductal cell when no more than 25%, 30%, 35%, 40%, 45%, or 50% of the CpG sites are unmethylated. In some embodiments, the method identifies the target DNA fragment as not being from a pancreatic ductal cell when no more than 25%, 30%, 35%, 40%, 45%, or 50% of the CpG sites are methylated. In some embodiments, the method identifies the target DNA fragment as not being from a pancreatic ductal cell when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 1639-1658, 1659-1742, 1743-1743, 1744-1747, 1748-1751, 1752-1767, 1768-1779 or 1780-1792.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a pancreatic ductal cell of a subject, the method indicates that the subject has abnormal cell death and/or a disease relating to the cell. In some embodiments, the disease or condition is injury, inflammation, or cancer of the pancreatic ductal cells. In some embodiments, the disease is diabetes.

In some embodiments, when the amount of cell-free DNA identified as being from a particular type of cell or cells, e.g., pancreatic ductal cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., pancreatic ductal cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a pancreatic ductal cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### Group 1—GI Epithelium (Colon Epithelium & Gastric Epithelium & Small Intestine Epithelium)

Also as provided in Table A, some genomic locations are uniformly under-methylated or over-methylated in a group of cells, namely colon epithelium & gastric epithelium & small intestine epithelium, as compared to all other cell types in the human.

In accordance with one embodiment of the present disclosure, a method is provided for identifying that a biological sample includes DNA from a cell selected from colon epithelium & gastric epithelium & small intestine epithelium. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 6541-6556, 6557-6557 or 6558-6565, or selected from SEQ ID NO: 6541-6556. In some embodiments, the method then identifies the target DNA fragment as being from a cell selected from colon epithelium & gastric epithelium & small intestine epithelium when no more than 40% of the CpG sites are methylated. In some embodiments, the method identifies the target DNA fragment as being from a cell selected from colon epithelium & gastric epithelium & small intestine epithelium when no more than 25%, 30%, 35%, 40%, 45%, or 50% of the CpG sites are methylated. Likewise, in some embodiments, the method identifies the target DNA fragment as being from a cell selected from colon epithelium & gastric epithelium & small intestine epithelium when at least 50%, 55%, 60%, 65%, 70%, 75%, 80%, 85% or 90% of the CpG sites are unmethylated. In some embodiments, the method identifies the target DNA fragment as not being from a cell selected from colon epithelium & gastric epithelium & small intestine epithelium when at least 50%, 55%, 60%, 65%, 70%, 75%, 80%, 85% or 90% of the CpG sites are methylated. In some embodiments, the method identifies the target DNA fragment as not being from a cell selected from colon epithelium & gastric epithelium & small intestine epithelium when no more than 25%, 30%, 35%, 40%, 45%, or 50% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 6541-6556, 6557-6557 or 6558-6565.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a cell selected from colon epithelium & gastric epithelium & small intestine epithelium of a subject, the method indicates that the subject has abnormal cell death and/or a disease relating to the cell. In some embodiments, the disease or condition is injury, inflammation, or cancer of a cell selected from colon epithelium & gastric epithelium & small intestine epithelium.

In some embodiments, when the amount of cell-free DNA identified as being from a particular type of cell or cells, e.g., cells selected from colon epithelium & gastric epithelium & small intestine epithelium, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., cells selected from colon epithelium & gastric epithelium & small intestine epithelium is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a cell selected from colon epithelium & gastric epithelium & small intestine epithelium, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### Group 2—Small Intestine Epithelium & Colon Epithelium

Also as provided in Table A, some genomic locations are uniformly under-methylated or over-methylated in a group of cells, namely small intestine epithelium & colon epithelium, as compared to all other cell types in the human.

In accordance with one embodiment of the present disclosure, a method is provided for identifying that a biological sample includes DNA from a cell selected from small intestine epithelium & colon epithelium. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 6695-6702, 6703-6760, 6761-6777 or 6778-6820, or selected from SEQ ID NO: 6695-6702 or 6703-6760. In some embodiments, the method then identifies the target DNA fragment as being from a cell selected from small intestine epithelium & colon epithelium when no more than 40% of the CpG sites are methylated. In some embodiments, the method identifies the target DNA fragment as being from a cell selected from small intestine epithelium & colon epithelium when no more than 25%, 30%, 35%, 40%, 45%, or 50% of the CpG sites are methylated. Likewise, in some embodiments, the method identifies the target DNA fragment as being from a cell selected from small intestine epithelium & colon epithelium when at least 50%, 55%, 60%, 65%, 70%, 75%, 80%, 85% or 90% of the CpG sites are unmethylated. In some embodiments, the method identifies the target DNA fragment as not being from a cell selected from small intestine epithelium & colon epithelium when at least 50%, 55%, 60%, 65%, 70%, 75%, 80%, 85% or 90% of the CpG sites are methylated. In some embodiments, the method identifies the target DNA fragment as not being from a cell selected from small intestine epithelium & colon epithelium when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 6821-6825 or 6826-6845, or selected from SEQ ID NO: 6821-6825. In some embodiments, the method then identifies the target DNA fragment as being from a cell selected from small intestine epithelium & colon epithelium when 50% or more of the CpG sites are methylated. In some embodiments, the method identifies the target DNA fragment as being from a cell selected from small intestine epithelium & colon epithelium when at least 55%, 60%, 65%, 70%, 75%, 80%, 85% or 90% of the CpG sites are methylated. Likewise, in some embodiments, the method identifies the target DNA fragment as being from a cell selected from small intestine epithelium & colon epithelium when no more than 25%, 30%, 35%, 40%, 45%, or 50% of the CpG sites are unmethylated. In some embodiments, the method identifies the target DNA fragment as not being from a cell selected from small intestine epithelium & colon epithelium when no more than 25%, 30%, 35%, 40%, 45%, or 50% of the CpG sites are methylated. In some embodiments, the method identifies the target DNA fragment as not being from a cell selected from small intestine epithelium & colon epithelium when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 6695-6702, 6703-6760, 6761-6777, 6778-6820, 6821-6825 or 6826-6845.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a cell selected from small intestine epithelium & colon epithelium of a subject, the method indicates that the subject has abnormal cell death and/or a disease relating to the cell. In some embodiments, the disease or condition is injury, inflammation, or cancer of a cell selected from small intestine epithelium & colon epithelium.

In some embodiments, when the amount of cell-free DNA identified as being from a particular type of cell or cells, e.g., cells selected from small intestine epithelium & colon epithelium, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., cells selected from small intestine epithelium & colon epithelium is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a cell selected from small intestine epithelium & colon epithelium, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### Group 3—Gastric Epithelium & Small Intestine Epithelium

Also as provided in Table A, some genomic locations are uniformly under-methylated or over-methylated in a group of cells, namely gastric epithelium & small intestine epithelium, as compared to all other cell types in the human.

In accordance with one embodiment of the present disclosure, a method is provided for identifying that a biological sample includes DNA from a cell selected from gastric epithelium & small intestine epithelium. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 6566-6589, 6590-6672, 6673-6673, 6674-6674 or 6675-6690, or selected from SEQ ID NO: 6566-6589 or 6590-6672. In some embodiments, the method then identifies the target DNA fragment as being from a cell selected from gastric epithelium & small intestine epithelium when no more than 40% of the CpG sites are methylated. In some embodiments, the method identifies the target DNA fragment as being from a cell selected from gastric epithelium & small intestine epithelium when no more than 25%, 30%, 35%, 40%, 45%, or 50% of the CpG sites are methylated. Likewise, in some embodiments, the method identifies the target DNA fragment as being from a cell selected from gastric epithelium & small intestine epithelium when at least 50%, 55%, 60%, 65%, 70%, 75%, 80%, 85% or 90% of the CpG sites are unmethylated. In some embodiments, the method identifies the target DNA fragment as not being from a cell selected from gastric epithelium & small intestine epithelium when at least 50%, 55%, 60%, 65%, 70%, 75%, 80%, 85% or 90% of the CpG sites are methylated. In some embodiments, the method identifies the target DNA fragment as not being from a cell selected from gastric epithelium & small intestine epithelium when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 6691 or 6692-6694, or selected from SEQ ID NO: 6691. In some embodiments, the method then identifies the target DNA fragment as being from a cell selected from gastric epithelium & small intestine epithelium when 50% or more of the CpG sites are methylated. In some embodiments, the method identifies the target DNA fragment as being from a cell selected from gastric epithelium & small intestine epithelium when at least 55%, 60%, 65%, 70%, 75%, 80%, 85% or 90% of the CpG sites are methylated. Likewise, in some embodiments, the method identifies the target DNA fragment as being from a cell selected from gastric epithelium & small intestine epithelium when no more than 25%, 30%, 35%, 40%, 45%, or 50% of the CpG sites are unmethylated. In some embodiments, the method identifies the target DNA fragment as not being from a cell selected from gastric epithelium & small intestine epithelium when no more than 25%, 30%, 35%, 40%, 45%, or 50% of the CpG sites are methylated. In some embodiments, the method identifies the target DNA fragment as not being from a cell selected from gastric epithelium & small intestine epithelium when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 6566-6589, 6590-6672, 6673-6673, 6674-6674, 6675-6690, 6691 or 6692-6694.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a cell selected from gastric epithelium & small intestine epithelium of a subject, the method indicates that the subject has abnormal cell death and/or a disease relating to the cell. In some embodiments, the disease or condition is injury, inflammation, or cancer of a cell selected from gastric epithelium & small intestine epithelium.

In some embodiments, when the amount of cell-free DNA identified as being from a particular type of cell or cells, e.g., cells selected from gastric epithelium & small intestine epithelium, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., cells selected from gastric epithelium & small intestine epithelium is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a cell selected from gastric epithelium & small intestine epithelium, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### Group 4—Colonfibroblasts & Heart Fibroblasts

Also as provided in Table A, some genomic locations are uniformly under-methylated or over-methylated in a group of cells, namely colon fibroblasts & heart fibroblasts, as compared to all other cell types in the human.

In accordance with one embodiment of the present disclosure, a method is provided for identifying that a biological sample includes DNA from a cell selected from colon fibroblasts & heart fibroblasts. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 6846-6863, 6864-6869, 6870-6872, 6873-6876 or 6877-6878, or selected from SEQ ID NO: 6846-6863 or 6864-6869. In some embodiments, the method then identifies the target DNA fragment as being from a cell selected from colon fibroblasts & heart fibroblasts when no more than 40% of the CpG sites are methylated. In some embodiments, the method identifies the target DNA fragment as being from a cell selected from colon fibroblasts & heart fibroblasts when no more than 25%, 30%, 35%, 40%, 45%, or 50% of the CpG sites are methylated. Likewise, in some embodiments, the method identifies the target DNA fragment as being from a cell selected from colon fibroblasts & heart fibroblasts when at least 50%, 55%, 60%, 65%, 70%, 75%, 80%, 85% or 90% of the CpG sites are unmethylated. In some embodiments, the method identifies the target DNA fragment as not being from a cell selected from colon fibroblasts & heart fibroblasts when at least 50%, 55%, 60%, 65%, 70%, 75%, 80%, 85% or 90% of the CpG sites are methylated. In some embodiments, the method identifies the target DNA fragment as not being from a cell selected from colon fibroblasts & heart fibroblasts when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 6879-6890 or 6891-6898, or selected from SEQ ID NO: 6879-6890. In some embodiments, the method then identifies the target DNA fragment as being from a cell selected from colon fibroblasts & heart fibroblasts when 50% or more of the CpG sites are methylated. In some embodiments, the method identifies the target DNA fragment as being from a cell selected from colon fibroblasts & heart fibroblasts when at least 55%, 60%, 65%, 70%, 75%, 80%, 85% or 90% of the CpG sites are methylated. Likewise, in some embodiments, the method identifies the target DNA fragment as being from a cell selected from colon fibroblasts & heart fibroblasts when no more than 25%, 30%, 35%, 40%, 45%, or 50% of the CpG sites are unmethylated. In some embodiments, the method identifies the target DNA fragment as not being from a cell selected from colon fibroblasts & heart fibroblasts when no more than 25%, 30%, 35%, 40%, 45%, or 50% of the CpG sites are methylated. In some embodiments, the method identifies the target DNA fragment as not being from a cell selected from colon fibroblasts & heart fibroblasts when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 6846-6863, 6864-6869, 6870-6872, 6873-6876, 6877-6878, 6879-6890 or 6891-6898.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a cell selected from colon fibroblasts & heart fibroblasts of a subject, the method indicates that the subject has abnormal cell death and/or a disease relating to the cell. In some embodiments, the disease or condition is injury, inflammation, or cancer of a cell selected from colon fibroblasts & heart fibroblasts.

In some embodiments, when the amount of cell-free DNA identified as being from a particular type of cell or cells, e.g., cells selected from colon fibroblasts & heart fibroblasts, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., cells selected from colon fibroblasts & heart fibroblasts is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a cell selected from colon fibroblasts & heart fibroblasts, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### Group 5 Pancreatic Alpha & Beta & Delta Cells

Also as provided in Table A, some genomic locations are uniformly under-methylated or over-methylated in a group of cells, namely pancreatic alpha & beta & delta cells, as compared to all other cell types in the human.

In accordance with one embodiment of the present disclosure, a method is provided for identifying that a biological sample includes DNA from a cell selected from pancreatic alpha & beta & delta cells. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 5924-5935, 5936-6011, 6012-6012, 6013-6014, 6015-6026 or 6027-6050, or selected from SEQ ID NO: 5924-5935 or 5936-6011. In some embodiments, the method then identifies the target DNA fragment as being from a cell selected from pancreatic alpha & beta & delta cells when no more than 40% of the CpG sites are methylated. In some embodiments, the method identifies the target DNA fragment as being from a cell selected from pancreatic alpha & beta & delta cells when no more than 25%, 30%, 35%, 40%, 45%, or 50% of the CpG sites are methylated. Likewise, in some embodiments, the method identifies the target DNA fragment as being from a cell selected from pancreatic alpha & beta & delta cells when at least 50%, 55%, 60%, 65%, 70%, 75%, 80%, 85% or 90% of the CpG sites are unmethylated. In some embodiments, the method identifies the target DNA fragment as not being from a cell selected from pancreatic alpha & beta & delta cells when at least 50%, 55%, 60%, 65%, 70%, 75%, 80%, 85% or 90% of the CpG sites are methylated. In some embodiments, the method identifies the target DNA fragment as not being from a cell selected from pancreatic alpha & beta & delta cells when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 6051-6057 or 6058-6075, or selected from SEQ ID NO: 6051-6057. In some embodiments, the method then identifies the target DNA fragment as being from a cell selected from pancreatic alpha & beta & delta cells when 50% or more of the CpG sites are methylated. In some embodiments, the method identifies the target DNA fragment as being from a cell selected from pancreatic alpha & beta & delta cells when at least 55%, 60%, 65%, 70%, 75%, 80%, 85% or 90% of the CpG sites are methylated. Likewise, in some embodiments, the method identifies the target DNA fragment as being from a cell selected from pancreatic alpha & beta & delta cells when no more than 25%, 30%, 35%, 40%, 45%, or 50% of the CpG sites are unmethylated. In some embodiments, the method identifies the target DNA fragment as not being from a cell selected from pancreatic alpha & beta & delta cells when no more than 25%, 30%, 35%, 40%, 45%, or 50%, 16 of the CpG sites are methylated. In some embodiments, the method identifies the target DNA fragment as not being from a cell selected from pancreatic alpha & beta & delta cells when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 5924-5935, 5936-6011, 6012-6012, 6013-6014, 6015-6026, 6027-6050, 6051-6057 or 6058-6075.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a cell selected from pancreatic alpha & beta & delta cells of a subject, the method indicates that the subject has abnormal cell death and/or a disease relating to the cell. In some embodiments, the disease or condition is injury, inflammation, or cancer of a cell selected from pancreatic alpha & beta & delta cells.

In some embodiments, when the amount of cell-free DNA identified as being from a particular type of cell or cells, e.g., cells selected from pancreatic alpha & beta & delta cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., cells selected from pancreatic alpha & beta & delta cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a cell selected from pancreatic alpha & beta & delta cells, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### B. Genito-Urinary Cells

### B1. Endometrium Epithelium

Also as provided in Table A, some genomic locations are uniformly under-methylated or over-methylated in endometrium epithelial cells as compared to all other cell types in the human.

In accordance with one embodiment of the present disclosure, a method is provided for identifying that a biological sample includes DNA from an endometrium epithelial cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 1793-1864, 1865-1872 or 1873-1892, or selected from SEQ ID NO: 1793-1864. In some embodiments, the method then identifies the target DNA fragment as being from an endometrium epithelial cell when no more than 40% of the CpG sites are methylated. In some embodiments, the method identifies the target DNA fragment as being from an endometrium epithelial cell when no more than 25%, 30%, 35%, 40%, 45%, or 50% of the CpG sites are methylated. Likewise, in some embodiments, the method identifies the target DNA fragment as being from an endometrium epithelial cell when at least 50%, 55%, 60%, 65%, 70%, 75%, 80%, 85% or 90% of the CpG sites are unmethylated. In some embodiments, the method identifies the target DNA fragment as not being from an endometrium epithelial cell when at least 50%, 55%, 60%, 65%, 70%, 75%, 80%, 85% or 90% of the CpG sites are methylated. In some embodiments, the method identifies the target DNA fragment as not being from an endometrium epithelial cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 1893-1905 or 1906-1917, or selected from SEQ ID NO: 1893-1905. In some embodiments, the method then identifies the target DNA fragment as being from an endometrium epithelial cell when 50% or more of the CpG sites are methylated. In some embodiments, the method identifies the target DNA fragment as being from an endometrium epithelial cell when at least 55%, 60%, 65%, 70%, 75%, 80%, 85% or 90% of the CpG sites are methylated. Likewise, in some embodiments, the method identifies the target DNA fragment as being from an endometrium epithelial cell when no more than 25%, 30%, 35%, 40%, 45%, or 50% of the CpG sites are unmethylated. In some embodiments, the method identifies the target DNA fragment as not being from an endometrium epithelial cell when no more than 25%, 30%, 35%, 40%, 45%, or 50% of the CpG sites are methylated. In some embodiments, the method identifies the target DNA fragment as not being from an endometrium epithelial cell when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 1793-1864, 1865-1872, 1873-1892, 1893-1905 or 1906-1917.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from an endometrium epithelial cell of a subject, the method indicates that the subject has abnormal cell death and/or a disease relating to the cell. In some embodiments, the disease or condition is injury, inflammation, or cancer of the endometrium epithelium.

In some embodiments, when the amount of cell-free DNA identified as being from a particular type of cell or cells, e.g., endometrium epithelial cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., endometrium epithelial cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as an endometrium epithelial cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### B2. Fallopian Epithelium

Also as provided in Table A, some genomic locations are uniformly under-methylated or over-methylated in fallopian epithelial cells as compared to all other cell types in the human.

In accordance with one embodiment of the present disclosure, a method is provided for identifying that a biological sample includes DNA from a fallopian epithelial cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 1918-1937, 1938-2022, 2023-2024, 2025-2029 or 2030-2042, or selected from SEQ ID NO: 1918-1937 or 1938-2022. In some embodiments, the method then identifies the target DNA fragment as being from a fallopian epithelial cell when no more than 40% of the CpG sites are methylated. In some embodiments, the method identifies the target DNA fragment as being from a fallopian epithelial cell when no more than 25%, 30%, 35%, 40%, 45%, or 50% of the CpG sites are methylated. Likewise, in some embodiments, the method identifies the target DNA fragment as being from a fallopian epithelial cell when at least 50%, 55%, 60%, 65%, 70%, 75%, 80%, 85% or 90% of the CpG sites are unmethylated. In some embodiments, the method identifies the target DNA fragment as not being from a fallopian epithelial cell when at least 50%, 55%, 60%, 65%, 70%, 75%, 80%, 85% or 90% of the CpG sites are methylated. In some embodiments, the method identifies the target DNA fragment as not being from a fallopian epithelial cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 2043-2061 or 2062-2067, or selected from SEQ ID NO: 2043-2061. In some embodiments, the method then identifies the target DNA fragment as being from a fallopian epithelial cell when 50% or more of the CpG sites are methylated. In some embodiments, the method identifies the target DNA fragment as being from a fallopian epithelial cell when at least 55%, 60%, 65%, 70%, 75%, 80%, 85% or 90% of the CpG sites are methylated. Likewise, in some embodiments, the method identifies the target DNA fragment as being from a fallopian epithelial cell when no more than 25%, 30%, 35%, 40%, 45%, or 50% of the CpG sites are unmethylated. In some embodiments, the method identifies the target DNA fragment as not being from a fallopian epithelial cell when no more than 25%, 30%, 35%, 40%, 45%, or 50% of the CpG sites are methylated. In some embodiments, the method identifies the target DNA fragment as not being from a fallopian epithelial cell when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 1918-1937, 1938-2022, 2023-2024, 2025-2029, 2030-2042, 2043-2061 or 2062-2067.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a fallopian epithelial cell of a subject, the method indicates that the subject has abnormal cell death and/or a disease relating to the cell. In some embodiments, the disease or condition is injury, inflammation, or cancer of the fallopian epithelium.

In some embodiments, when the amount of cell-free DNA identified as being from a particular type of cell or cells, e.g., fallopian epithelial cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., fallopian epithelial cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a fallopian epithelial cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### B3. Kidney Epithelium

Also as provided in Table A, some genomic locations are uniformly under-methylated or over-methylated in kidney epithelial cells as compared to all other cell types in the human.

In accordance with one embodiment of the present disclosure, a method is provided for identifying that a biological sample includes DNA from a kidney epithelial cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 2068-2080, 2081-2141, 2142-2144, 2145-2156 or 2157-2194, or selected from SEQ ID NO: 2068-2080 or 2081-2141. In some embodiments, the method then identifies the target DNA fragment as being from a kidney epithelial cell when no more than 40% of the CpG sites are methylated. In some embodiments, the method identifies the target DNA fragment as being from a kidney epithelial cell when no more than 25%, 30%, 35%, 40%, 45%, or 50% of the CpG sites are methylated. Likewise, in some embodiments, the method identifies the target DNA fragment as being from a kidney epithelial cell when at least 50%, 55%, 60%, 65%, 70%, 75%, 80%, 85% or 90% of the CpG sites are unmethylated. In some embodiments, the method identifies the target DNA fragment as not being from a kidney epithelial cell when at least 50%, 55%, 60%, 65%, 70%, 75%, 80%, 85% or 90% of the CpG sites are methylated. In some embodiments, the method identifies the target DNA fragment as not being from a kidney epithelial cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 2195-2209 or 2210-2219, or selected from SEQ ID NO: 2195-2209. In some embodiments, the method then identifies the target DNA fragment as being from a kidney epithelial cell when 50% or more of the CpG sites are methylated. In some embodiments, the method identifies the target DNA fragment as being from a kidney epithelial cell when at least 55%, 60%, 65%, 70%, 75%, 80%, 85% or 90% of the CpG sites are methylated. Likewise, in some embodiments, the method identifies the target DNA fragment as being from a kidney epithelial cell when no more than 25%, 30%, 35%, 40%, 45%, or 50% of the CpG sites are unmethylated. In some embodiments, the method identifies the target DNA fragment as not being from a kidney epithelial cell when no more than 25%, 30%, 35%, 40%, 45%, or 50% of the CpG sites are methylated. In some embodiments, the method identifies the target DNA fragment as not being from a kidney epithelial cell when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 2068-2080, 2081-2141, 2142-2144, 2145-2156, 2157-2194, 2195-2209 or 2210-2219.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a kidney epithelial cell of a subject, the method indicates that the subject has abnormal cell death and/or a disease relating to the cell. In some embodiments, the disease or condition is injury, inflammation, or cancer of the kidney epithelium.

In some embodiments, when the amount of cell-free DNA identified as being from a particular type of cell or cells, e.g., kidney epithelial cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., kidney epithelial cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a kidney epithelial cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### B4. Bladder Epithelium

Also as provided in Table A, some genomic locations are uniformly under-methylated or over-methylated in bladder epithelial cells as compared to all other cell types in the human.

In accordance with one embodiment of the present disclosure, a method is provided for identifying that a biological sample includes DNA from a bladder epithelial cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 2220-2233, 2234-2298, 2299-2299, 2300-2303, 2304-2313 or 2314-2345, or selected from SEQ ID NO: 2220-2233 or 2234-2298. In some embodiments, the method then identifies the target DNA fragment as being from a bladder epithelial cell when no more than 40% of the CpG sites are methylated. In some embodiments, the method identifies the target DNA fragment as being from a bladder epithelial cell when no more than 25%, 30%, 35%, 40%, 45%, or 50% of the CpG sites are methylated. Likewise, in some embodiments, the method identifies the target DNA fragment as being from a bladder epithelial cell when at least 50%, 55%, 60%, 65%, 70%, 75%, 80%, 85% or 90% of the CpG sites are unmethylated. In some embodiments, the method identifies the target DNA fragment as not being from a bladder epithelial cell when at least 50%, 55%, 60%, 65%, 70%, 75%, 80%, 85% or 90% of the CpG sites are methylated. In some embodiments, the method identifies the target DNA fragment as not being from a bladder epithelial cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 2346-2350, 2351-2351 or 2352-2370, or selected from SEQ ID NO: 2346-2350. In some embodiments, the method then identifies the target DNA fragment as being from a bladder epithelial cell when 50% or more of the CpG sites are methylated. In some embodiments, the method identifies the target DNA fragment as being from a bladder epithelial cell when at least 55%, 60%, 65%, 70%, 75%, 80%, 85% or 90% of the CpG sites are methylated. Likewise, in some embodiments, the method identifies the target DNA fragment as being from a bladder epithelial cell when no more than 25%, 30%, 35%, 40%, 45%, or 50% of the CpG sites are unmethylated. In some embodiments, the method identifies the target DNA fragment as not being from a bladder epithelial cell when no more than 25%, 30%, 35%, 40%, 45%, or 50% of the CpG sites are methylated. In some embodiments, the method identifies the target DNA fragment as not being from a bladder epithelial cell when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 2220-2233, 2234-2298, 2299-2299, 2300-2303, 2304-2313, 2314-2345, 2346-2350, 2351-2351 or 2352-2370.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a bladder epithelial cell of a subject, the method indicates that the subject has abnormal cell death and/or a disease relating to the cell. In some embodiments, the disease or condition is injury, inflammation, or cancer of the bladder epithelium.

In some embodiments, when the amount of cell-free DNA identified as being from a particular type of cell or cells, e.g., bladder epithelial cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., bladder epithelial cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a bladder epithelial cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### B5. Prostate Epithelium

Also as provided in Table A, some genomic locations are uniformly under-methylated or over-methylated in prostate epithelial cells as compared to all other cell types in the human.

In accordance with one embodiment of the present disclosure, a method is provided for identifying that a biological sample includes DNA from a prostate epithelial cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 2371-2389, 2390-2476, 2477-2480, 2481-2486 or 2487-2495, or selected from SEQ 1D NO: 2371-2389 or 2390-2476. In some embodiments, the method then identifies the target DNA fragment as being from a prostate epithelial cell when no more than 40% of the CpG sites are methylated. In some embodiments, the method identifies the target DNA fragment as being from a prostate epithelial cell when no more than 25%, 30%, 35%, 40%, 45%, or 50% of the CpG sites are methylated. Likewise, in some embodiments, the method identifies the target DNA fragment as being from a prostate epithelial cell when at least 50%, 55%, 60%, 65%, 70%, 75%, 80%, 85% or 90% of the CpG sites are unmethylated. In some embodiments, the method identifies the target DNA fragment as not being from a prostate epithelial cell when at least 50%, 55%, 60%, 65%, 70%, 75%, 80%, 85% or 90% of the CpG sites are methylated. In some embodiments, the method identifies the target DNA fragment as not being from a prostate epithelial cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 2496-2500, 2501-2501 or 2502-2520, or selected from SEQ ID NO: 2496-2500. In some embodiments, the method then identifies the target DNA fragment as being from a prostate epithelial cell when 50% or more of the CpG sites are methylated. In some embodiments, the method identifies the target DNA fragment as being from a prostate epithelial cell when at least 55%, 60%, 65%, 70%, 75%, 80%, 85% or 90% of the CpG sites are methylated. Likewise, in some embodiments, the method identifies the target DNA fragment as being from a prostate epithelial cell when no more than 25%, 30%, 35%, 40%, 45%, or 50% of the CpG sites are unmethylated. In some embodiments, the method identifies the target DNA fragment as not being from a prostate epithelial cell when no more than 25%, 30%, 35%, 40%, 45%, or 50% of the CpG sites are methylated. In some embodiments, the method identifies the target DNA fragment as not being from a prostate epithelial cell when at least 50%, 55%, 60%, 65%, 70%, 75%, 800, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 2371-2389, 2390-2476, 2477-2480, 2481-2486, 2487-2495, 2496-2500, 2501-2501 or 2502-2520.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a prostate epithelial cell of a subject, the method indicates that the subject has abnormal cell death and/or a disease relating to the cell. In some embodiments, the disease or condition is injury, inflammation, or cancer of the prostate epithelium.

In some embodiments, when the amount of cell-free DNA identified as being from a particular type of cell or cells, e.g., prostate epithelial cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., prostate epithelial cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a prostate epithelial cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### B6. Breast Basal Epithelium

Also as provided in Table A, some genomic locations are uniformly under-methylated or over-methylated in breast basal epithelial cells as compared to all other cell types in the human.

In accordance with one embodiment of the present disclosure, a method is provided for identifying that a biological sample includes DNA from a breast basal epithelial cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 2521-2536, 2537-2616, 2617-2625 or 2626-2651, or selected from SEQ ID NO: 2521-2536 or 2537-2616. In some embodiments, the method then identifies the target DNA fragment as being from a breast basal epithelial cell when no more than 40% of the CpG sites are methylated. In some embodiments, the method identifies the target DNA fragment as being from a breast basal epithelial cell when no more than 25%, 30%, 35%, 40%, 45%, or 50% of the CpG sites are methylated. Likewise, in some embodiments, the method identifies the target DNA fragment as being from a breast basal epithelial cell when at least 50%, 55%, 60%, 65%, 70%, 75%, 80%, 85% or 90% of the CpG sites are unmethylated. In some embodiments, the method identifies the target DNA fragment as not being from a breast basal epithelial cell when at least 50%, 55%, 60%, 65%, 70%, 75%, 80%, 85% or 90% of the CpG sites are methylated. In some embodiments, the method identifies the target DNA fragment as not being from a breast basal epithelial cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 2652-2659 or 2660-2676, or selected from SEQ ID NO: 2652-2659. In some embodiments, the method then identifies the target DNA fragment as being from a breast basal epithelial cell when 50% or more of the CpG sites are methylated. In some embodiments, the method identifies the target DNA fragment as being from a breast basal epithelial cell when at least 55%, 60%, 65%, 70%, 75%, 80%, 85% or 90% of the CpG sites are methylated. Likewise, in some embodiments, the method identifies the target DNA fragment as being from a breast basal epithelial cell when no more than 25%, 30%, 35%, 40%, 45%, or 50% of the CpG sites are unmethylated. In some embodiments, the method identifies the target DNA fragment as not being from a breast basal epithelial cell when no more than 25%, 30%, 35%, 40%, 45%, or 50% of the CpG sites are methylated. In some embodiments, the method identifies the target DNA fragment as not being from a breast basal epithelial cell when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 2521-2536, 2537-2616, 2617-2625, 2626-2651, 2652-2659 or 2660-2676.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a breast basal epithelial cell of a subject, the method indicates that the subject has abnormal cell death and/or a disease relating to the cell. In some embodiments, the disease or condition is injury, inflammation, or cancer of the breast basal epithelium.

In some embodiments, when the amount of cell-free DNA identified as being from a particular type of cell or cells, e.g., breast basal epithelial cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., breast basal epithelial cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a breast basal epithelial cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### B7. Breast Luminal Epithelium

Also as provided in Table A, some genomic locations are uniformly under-methylated or over-methylated in breast luminal epithelial cells as compared to all other cell types in the human.

In accordance with one embodiment of the present disclosure, a method is provided for identifying that a biological sample includes DNA from a breast luminal epithelial cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 2677-2688, 2689-2748, 2749-2749, 2750-2762 or 2763-2802, or selected from SEQ ID NO: 2677-2688 or 2689-2748. In some embodiments, the method then identifies the target DNA fragment as being from a breast luminal epithelial cell when no more than 40% of the CpG sites are methylated. In some embodiments, the method identifies the target DNA fragment as being from a breast luminal epithelial cell when no more than 25%, 30%, 35%, 40%4, 45%, or 50% of the CpG sites are methylated. Likewise, in some embodiments, the method identifies the target DNA fragment as being from a breast luminal epithelial cell when at least 50%, 55%, 60%, 65%, 70%, 75%, 80%, 85% or 90% of the CpG sites are unmethylated. In some embodiments, the method identifies the target DNA fragment as not being from a breast luminal epithelial cell when at least 50%, 55%, 60%, 65%, 70%, 75%, 80%, 85% or 90% of the CpG sites are methylated. In some embodiments, the method identifies the target DNA fragment as not being from a breast luminal epithelial cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 2803-2815, 2816-2816 or 2817-2827, or selected from SEQ ID NO: 2803-2815. In some embodiments, the method then identifies the target DNA fragment as being from a breast luminal epithelial cell when 50% or more of the CpG sites are methylated. In some embodiments, the method identifies the target DNA fragment as being from a breast luminal epithelial cell when at least 55%, 60%, 65%, 70%, 75%, 80%, 85% or 90% of the CpG sites are methylated. Likewise, in some embodiments, the method identifies the target DNA fragment as being from a breast luminal epithelial cell when no more than 25%, 30%, 35%, 40%, 45%, or 50%, 10 of the CpG sites are unmethylated. In some embodiments, the method identifies the target DNA fragment as not being from a breast luminal epithelial cell when no more than 25%, 30%, 35%, 40%, 45%, or 50% of the CpG sites are methylated. In some embodiments, the method identifies the target DNA fragment as not being from a breast luminal epithelial cell when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 2677-2688, 2689-2748, 2749-2749, 2750-2762, 2763-2802, 2803-2815, 2816-2816 or 2817-2827.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a breast luminal epithelial cell of a subject, the method indicates that the subject has abnormal cell death and/or a disease relating to the cell. In some embodiments, the disease or condition is injury, inflammation, or cancer of the breast luminal epithelium.

In some embodiments, when the amount of cell-free DNA identified as being from a particular type of cell or cells, e.g., breast luminal epithelial cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., breast luminal epithelial cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a breast luminal epithelial cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### Group 6—Breast Basal Epithelium & Breast Luminal Epithelium

Also as provided in Table A, some genomic locations are uniformly under-methylated or over-methylated in a group of cells, namely breast basal epithelium & breast luminal epithelium, as compared to all other cell types in the human.

In accordance with one embodiment of the present disclosure, a method is provided for identifying that a biological sample includes DNA from a cell selected from breast basal epithelium & breast luminal epithelium. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 6076-6090, 6091-6159, 6160-6160, 6161-6162, 6163-6171 or 6172-6201, or selected from SEQ ID NO: 6076-6090 or 6091-6159. In some embodiments, the method then identifies the target DNA fragment as being from a cell selected from breast basal epithelium & breast luminal epithelium when no more than 40% of the CpG sites are methylated. In some embodiments, the method identifies the target DNA fragment as being from a cell selected from breast basal epithelium & breast luminal epithelium when no more than 25%, 30%, 35%, 40%, 45%, or 50% of the CpG sites are methylated. Likewise, in some embodiments, the method identifies the target DNA fragment as being from a cell selected from breast basal epithelium & breast luminal epithelium when at least 50%, 55%, 60%, 65%, 70%, 75%, 80%, 85% or 90% of the CpG sites are unmethylated. In some embodiments, the method identifies the target DNA fragment as not being from a cell selected from breast basal epithelium & breast luminal epithelium when at least 50%, 55%, 60%, 65%, 70%, 75%, 80%, 85% or 90% of the CpG sites are methylated. In some embodiments, the method identifies the target DNA fragment as not being from a cell selected from breast basal epithelium & breast luminal epithelium when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 6202-6206 or 6207-6226, or selected from SEQ ID NO: 6202-6206. In some embodiments, the method then identifies the target DNA fragment as being from a cell selected from breast basal epithelium & breast luminal epithelium when 50% or more of the CpG sites are methylated. In some embodiments, the method identifies the target DNA fragment as being from a cell selected from breast basal epithelium & breast luminal epithelium when at least 55%, 60%, 65%, 70%, 75%, 80%, 85% or 90% of the CpG sites are methylated. Likewise, in some embodiments, the method identifies the target DNA fragment as being from a cell selected from breast basal epithelium & breast luminal epithelium when no more than 25%, 30%, 35%, 40%, 45%, or 50% of the CpG sites are unmethylated. In some embodiments, the method identifies the target DNA fragment as not being from a cell selected from breast basal epithelium & breast luminal epithelium when no more than 25%, 30%, 35%, 40%, 45%, or 50% of the CpG sites are methylated. In some embodiments, the method identifies the target DNA fragment as not being from a cell selected from breast basal epithelium & breast luminal epithelium when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 6076-6090, 6091-6159, 6160-6160, 6161-6162, 6163-6171, 6172-6201, 6202-6206 or 6207-6226.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a cell selected from breast basal epithelium & breast luminal epithelium of a subject, the method indicates that the subject has abnormal cell death and/or a disease relating to the cell. In some embodiments, the disease or condition is injury, inflammation, or cancer of a cell selected from breast basal epithelium & breast luminal epithelium.

In some embodiments, when the amount of cell-free DNA identified as being from a particular type of cell or cells, e.g., cells selected from breast basal epithelium & breast luminal epithelium, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., cells selected from breast basal epithelium & breast luminal epithelium is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a cell selected from breast basal epithelium & breast luminal epithelium, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### Group 7—Fallopian Epithelium & Ovarian Epithelium & Endometrial Epithelium

Also as provided in Table A, some genomic locations are uniformly under-methylated or over-methylated in a group of cells, namely fallopian epithelium & ovarian epithelium & endometrial epithelium, as compared to all other cell types in the human.

In accordance with one embodiment of the present disclosure, a method is provided for identifying that a biological sample includes DNA from a cell selected from fallopian epithelium & ovarian epithelium & endometrial epithelium. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 6366-6399, 6400-6468, 6469-6475, 6476-6491 or 6492-6515, or selected from SEQ ID NO: 6366-6399 or 6400-6468. In some embodiments, the method then identifies the target DNA fragment as being from a cell selected from fallopian epithelium & ovarian epithelium & endometrial epithelium when no more than 40% of the CpG sites are methylated. In some embodiments, the method identifies the target DNA fragment as being from a cell selected from fallopian epithelium & ovarian epithelium & endometrial epithelium when no more than 25%, 30%, 35%, 40%, 45%, or 50% of the CpG sites are methylated. Likewise, in some embodiments, the method identifies the target DNA fragment as being from a cell selected from fallopian epithelium & ovarian epithelium & endometrial epithelium when at least 50%, 55%, 60%, 65%, 70%, 75%, 80%, 85% or 90% of the CpG sites are unmethylated. In some embodiments, the method identifies the target DNA fragment as not being from a cell selected from fallopian epithelium & ovarian epithelium & endometrial epithelium when at least 50%, 55%, 60%, 65%, 70%, 75%, 80%, 85% or 90% of the CpG sites are methylated. In some embodiments, the method identifies the target DNA fragment as not being from a cell selected from fallopian epithelium & ovarian epithelium & endometrial epithelium when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 6516-6527 or 6528-6540, or selected from SEQ ID NO: 6516-6527. In some embodiments, the method then identifies the target DNA fragment as being from a cell selected from fallopian epithelium & ovarian epithelium & endometrial epithelium when 50% or more of the CpG sites are methylated. In some embodiments, the method identifies the target DNA fragment as being from a cell selected from fallopian epithelium & ovarian epithelium & endometrial epithelium when at least 55%, 60%, 65%, 70%, 75%, 80%, 85% or 90% of the CpG sites are methylated. Likewise, in some embodiments, the method identifies the target DNA fragment as being from a cell selected from fallopian epithelium & ovarian epithelium & endometrial epithelium when no more than 25%, 30%, 35%, 40%, 45%, or 50% of the CpG sites are unmethylated. In some embodiments, the method identifies the target DNA fragment as not being from a cell selected from fallopian epithelium & ovarian epithelium & endometrial epithelium when no more than 25%, 30%, 16, 35%, 40%, 16, 45%, or 50% of the CpG sites are methylated. In some embodiments, the method identifies the target DNA fragment as not being from a cell selected from fallopian epithelium & ovarian epithelium & endometrial epithelium when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 6366-6399, 6400-6468, 6469-6475, 6476-6491, 6492-6515, 6516-6527 or 6528-6540.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a cell selected from fallopian epithelium & ovarian epithelium & endometrial epithelium of a subject, the method indicates that the subject has abnormal cell death and/or a disease relating to the cell. In some embodiments, the disease or condition is injury, inflammation, or cancer of a cell selected from fallopian epithelium & ovarian epithelium & endometrial epithelium.

In some embodiments, when the amount of cell-free DNA identified as being from a particular type of cell or cells, e.g., cells selected from fallopian epithelium & ovarian epithelium & endometrial epithelium, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., cells selected from fallopian epithelium & ovarian epithelium & endometrial epithelium is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a cell selected from fallopian epithelium & ovarian epithelium & endometrial epithelium, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### C. Cardio-Vascular-Pulmonary Cells

### C1. Lung Alveolar Epithelium

Also as provided in Table A, some genomic locations are uniformly under-methylated or over-methylated in lung alveolar epithelial cells as compared to all other cell types in the human.

In accordance with one embodiment of the present disclosure, a method is provided for identifying that a biological sample includes DNA from a lung alveolar epithelial cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 2828-2838, 2839-2899, 2900-2900, 2901-2903, 2904-2916 or 2917-2953, or selected from SEQ ID NO: 2828-2838 or 2839-2899. In some embodiments, the method then identifies the target DNA fragment as being from a lung alveolar epithelial cell when no more than 40% of the CpG sites are methylated. In some embodiments, the method identifies the target DNA fragment as being from a lung alveolar epithelial cell when no more than 25%, 30%, 35%, 40%, 45%, or 50% of the CpG sites are methylated. Likewise, in some embodiments, the method identifies the target DNA fragment as being from a lung alveolar epithelial cell when at least 50%, 55%, 60%, 65%, 70%, 75%, 80%, 85% or 90% of the CpG sites are unmethylated. In some embodiments, the method identifies the target DNA fragment as not being from a lung alveolar epithelial cell when at least 50%, 55%, 60%, 65%, 70%, 75%, 80%, 85% or 90% of the CpG sites are methylated. In some embodiments, the method identifies the target DNA fragment as not being from a lung alveolar epithelial cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 2954-2960 or 2961-2978, or selected from SEQ ID NO: 2954-2960. In some embodiments, the method then identifies the target DNA fragment as being from a lung alveolar epithelial cell when 50% or more of the CpG sites are methylated. In some embodiments, the method identifies the target DNA fragment as being from a lung alveolar epithelial cell when at least 55%, 60%, 65%, 70%, 75%, 80%, 85% or 90% of the CpG sites are methylated. Likewise, in some embodiments, the method identifies the target DNA fragment as being from a lung alveolar epithelial cell when no more than 25%, 30%, 35%, 40%, 45%, or 50% of the CpG sites are unmethylated. In some embodiments, the method identifies the target DNA fragment as not being from a lung alveolar epithelial cell when no more than 25%, 30%, 35%, 40%, 45%, or 50% of the CpG sites are methylated. In some embodiments, the method identifies the target DNA fragment as not being from a lung alveolar epithelial cell when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 2828-2838, 2839-2899, 2900-2900, 2901-2903, 2904-2916, 2917-2953, 2954-2960 or 2961-2978.

Example 2 of the instant disclosure discloses a set of methylation markers capable of distinguish different lung cell types, such as alveolar cells or bronchial cells. Example markers are provided in Table 3. The 17 genomic loci were uniquely unmethylated or hypermethylated in lung epithelial cells, including 3 loci that specifically identify bronchial cells, 12 loci that specifically identify alveolar cells, and 2 loci that can identify both of them. Using the reference chromosome locations as references, the 2 loci that identify both bronchial cells and alveolar cells are chromosome 14:55765534 (hg19, same below; reference gene. FBXO34) and chromosome 3:181441571 (reference gene: SOX2OT); the 12 loci that specifically identify alveolar cells are chromosome 1:41486102 (reference gene: SLFNL1), chromosome 2:236672684 (reference gene: AGAP1), chromosome 17:79952367 (reference gene: ASPSCR1), chromosome 16:678127 (reference gene: RAB40C), chromosome 7:2473529 (reference gene: CHST12), chromosome 16:1652552 (reference gene: IFT140), chromosome 14:91691190 (reference gene: C14orf159), chromosome 16:667157 (reference gene: RAB40C), chromosome 11:66116455 (reference gene: B3GNTI), chromosome 4:57522145 (reference gene: HOPX), chromosome 16:84271391 (reference gene: KCNG4), and chromosome 1:1986275 (reference gene: PRKCZ); the 3 loci that specifically identify bronchial cells are chromosome 7:4802132 (reference gene: FOXK1), chromosome 2:239970075 (reference gene: HDAC4), and chromosome 1:164761834 (reference gene: PBX1).

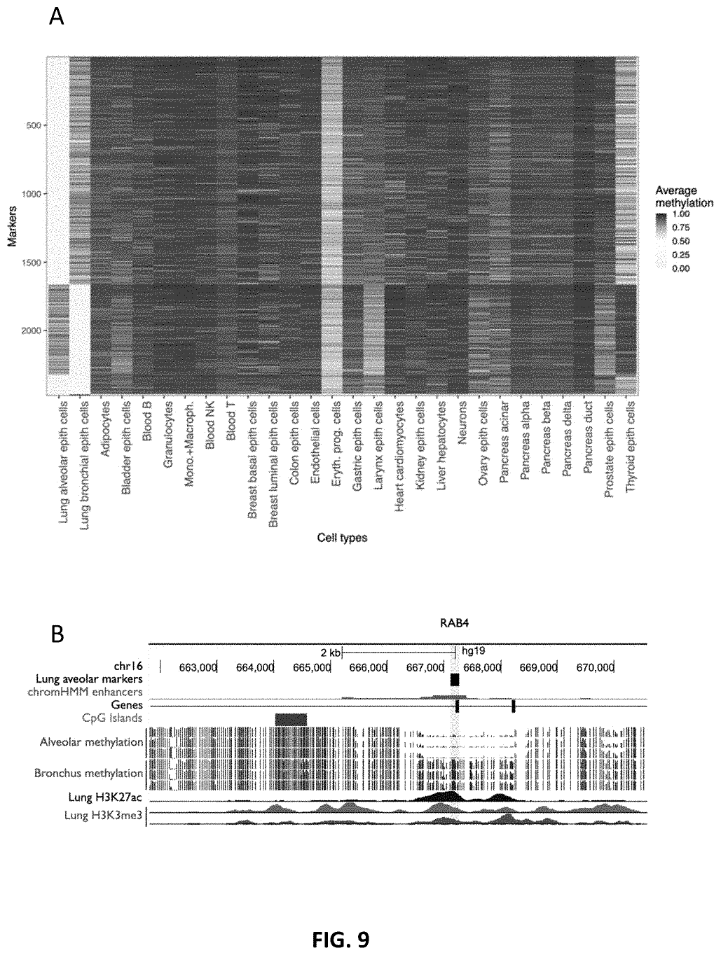

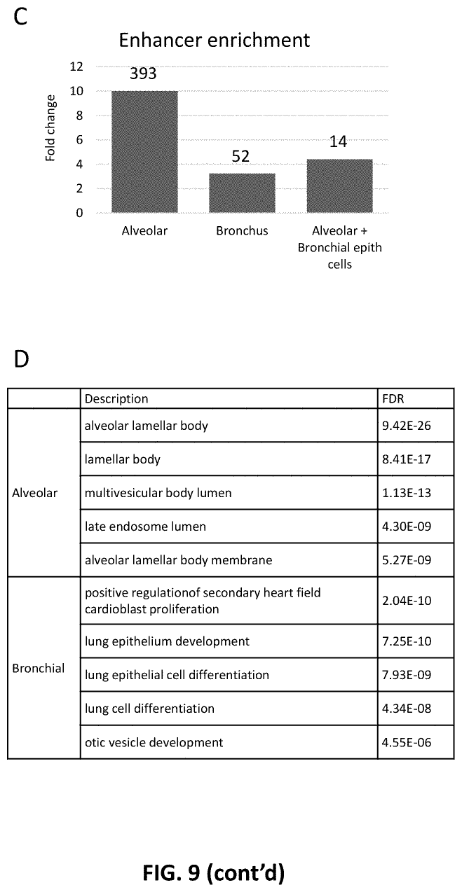

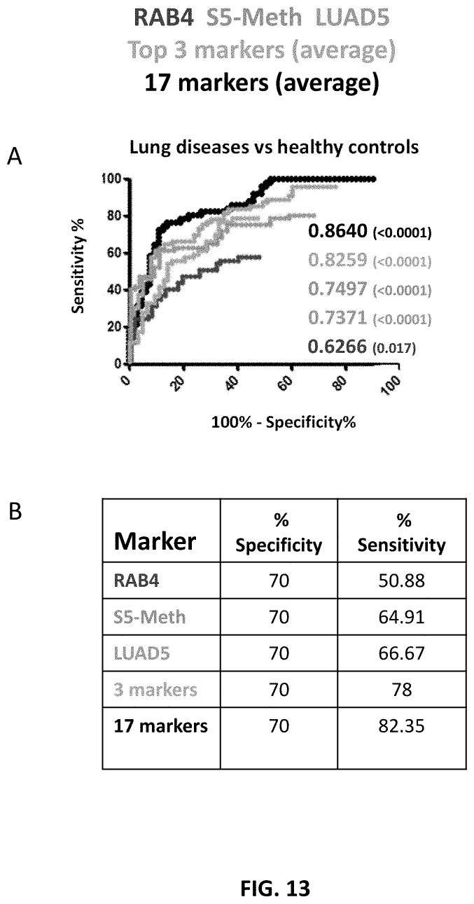

For instance, as shown in FIG. 9, the genomic marker sequence at the Rab40C gene was unmethylated only in lung alveolar epithelium, but not in bronchial cells. As demonstrated in FIG. 13, when the methylation status of one or more of these markers was used, the lung cell types could be readily distinguished. When the top three markers were used, the performance was close to when all 17 markers were used, underscoring the robustness of the technology.

Accordingly, in one embodiment, a method is provided for identifying that a biological sample comprises DNA from a lung cell, the method comprising detecting the methylation status of each of at least four CpG sites of a target DNA fragment in the biological sample; and identifying the target DNA fragment as being from a human lung alveolar cell or bronchial cell if the methylation status corresponds to a reference human lung alveolar cell or bronchial cell, wherein the target DNA fragment is within 1 kb from a genomic locus selected from the group selected from human chromosome 14:55765534, chromosome 3:181441571, chromosome 1:41486102, chromosome 2:236672684, chromosome 17:79952367, chromosome 16:678127, chromosome 7:2473529, chromosome 16:1652552, chromosome 14:91691190, chromosome 16:667157, chromosome 11:66116455, chromosome 4:57522145, chromosome 16:84271391, chromosome 1:1986275, chromosome 7:4802132, chromosome 2:239970075, chromosome 1:164761834, according to human genome assembly version hg19.

As used herein, in some embodiments, the methylation status refers to the percentage of CpG sites being methylated within the genomic sequence. In some embodiments, the methylation status simply refers to over-methylated (M, at least 60% CpG methylated) or under-methylated (U, no more than 40% CpG methylated).

For instance, in one embodiment, the target DNA fragment is identified as being from a human lung alveolar cell if target DNA fragment is unmethylated and is near a genomic locus of chromosome 2:236672684, chromosome 17:79952367, chromosome 16:678127, chromosome 7:2473529, chromosome 16:1652552, chromosome 14:91691190, chromosome 16:667157, chromosome 11:66116455, chromosome 16:84271391, or chromosome 1:1986275. In one embodiment, the target DNA fragment is identified as being from a human lung alveolar cell if target DNA fragment is methylated and is near a genomic locus of chromosome 4:57522145.

In one embodiment, the target DNA fragment is identified as being from a human lung bronchial cell if the target DNA fragment is unmethylated and is near a genomic locus of chromosome 7:4802132, chromosome 2:239970075, or chromosome 1:164761834.

In one embodiment, the target DNA fragment is identified as being from a human lung alveolar or bronchial cell if the target DNA fragment is unmethylated and is near a genomic locus of chromosome 14:55765534, or chromosome 1:41486102, or is methylated and is near a genomic locus of 3:181441571.

In some embodiments, the DNA fragment that contains the CpG sites used for measurement is within 1000 bp from the reference genomic location, e.g., chromosome 14:55765534. In some embodiments, the DNA fragment that contains the CpG sites used for measurement is within 900, 800, 700, 600, 500, 400, 300, 250, 200 or 150 bp from the reference genomic location.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a lung alveolar epithelial cell of a subject, the method indicates that the subject has abnormal cell death and/or a disease relating to the cell. In some embodiments, the disease or condition is injury, inflammation, or cancer of the lung alveolar epithelium.

In some embodiments, when the amount of cell-free DNA identified as being from a particular type of cell or cells, e.g., lung alveolar epithelial cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., lung alveolar epithelial cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a lung alveolar epithelial cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### C2. Lung Bronchial Epithelium

Also as provided in Table A, some genomic locations are uniformly under-methylated or over-methylated in lung bronchial epithelial cells as compared to all other cell types in the human.

In accordance with one embodiment of the present disclosure, a method is provided for identifying that a biological sample includes DNA from a lung bronchial epithelial cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 2979-3001, 3002-3087,3088-3090, 3091-3092 or 3093-3104, or selected from SEQ ID NO: 2979-3001 or 3002-3087. In some embodiments, the method then identifies the target DNA fragment as being from a lung bronchial epithelial cell when no more than 40% of the CpG sites are methylated. In some embodiments, the method identifies the target DNA fragment as being from a lung bronchial epithelial cell when no more than 25%, 30%, 35%, 40%, 45%, or 50% of the CpG sites are methylated. Likewise, in some embodiments, the method identifies the target DNA fragment as being from a lung bronchial epithelial cell when at least 50%, 55%, 60/o, 65%, 70%, 75%, 80%, 85% or 90% of the CpG sites are unmethylated. In some embodiments, the method identifies the target DNA fragment as not being from a lung bronchial epithelial cell when at least 50%, 55%, 60%/o, 65%, 70%, 75%, 80%, 85% or 90% of the CpG sites are methylated. In some embodiments, the method identifies the target DNA fragment as not being from a lung bronchial epithelial cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 3105-3109 or 3110-3129, or selected from SEQ ID NO: 3105-3109. In some embodiments, the method then identifies the target DNA fragment as being from a lung bronchial epithelial cell when 50% or more of the CpG sites are methylated. In some embodiments, the method identifies the target DNA fragment as being from a lung bronchial epithelial cell when at least 55%, 60%, 65%, 70%, 75%, 80%, 85% or 90% of the CpG sites are methylated. Likewise, in some embodiments, the method identifies the target DNA fragment as being from a lung bronchial epithelial cell when no more than 25%, 30%, 35%, 40%, 45%, or 50% of the CpG sites are unmethylated. In some embodiments, the method identifies the target DNA fragment as not being from a lung bronchial epithelial cell when no more than 25%, 30%, 35%, 40%, 45%, or 50% of the CpG sites are methylated. In some embodiments, the method identifies the target DNA fragment as not being from a lung bronchial epithelial cell when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 2979-3001, 3002-3087, 3088-3090, 3091-3092, 3093-3104, 3105-3109 or 3110-3129.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a lung bronchial epithelial cell of a subject, the method indicates that the subject has abnormal cell death and/or a disease relating to the cell. In some embodiments, the disease or condition is injury, inflammation, or cancer of the lung bronchial epithelium.

In some embodiments, when the amount of cell-free DNA identified as being from a particular type of cell or cells, e.g., lung bronchial epithelial cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., lung bronchial epithelial cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a lung bronchial epithelial cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### C3. Heart Cardiomyocytes

Also as provided in Table A, some genomic locations are uniformly under-methylated or over-methylated in heart cardiomyocytes as compared to all other cell types in the human.

In accordance with one embodiment of the present disclosure, a method is provided for identifying that a biological sample includes DNA from a heart cardiomyocyte. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 3130-3147, 3148-3223, 3224-3230 or 3231-3254, or selected from SEQ ID NO: 3130-3147 or 3148-3223. In some embodiments, the method then identifies the target DNA fragment as being from a heart cardiomyocyte when no more than 40% of the CpG sites are methylated. In some embodiments, the method identifies the target DNA fragment as being from a heart cardiomyocyte when no more than 25%, 30%, 35%, 40%, 45%, or 50% of the CpG sites are methylated. Likewise, in some embodiments, the method identifies the target DNA fragment as being from a heart cardiomyocyte when at least 50%, 55%, 60%, 65%, 70%, 75%, 80%, 85% or 90% of the CpG sites are unmethylated. In some embodiments, the method identifies the target DNA fragment as not being from a heart cardiomyocyte when at least 50%, 55%, 60%, 65%, 70%, 75%, 80%, 85% or 90% of the CpG sites are methylated. In some embodiments, the method identifies the target DNA fragment as not being from a heart cardiomyocyte when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 3255-3266, 3267-3267 or 3268-3279, or selected from SEQ ID NO: 3255-3266. In some embodiments, the method then identifies the target DNA fragment as being from a heart cardiomyocyte when 50% or more of the CpG sites are methylated. In some embodiments, the method identifies the target DNA fragment as being from a heart cardiomyocyte when at least 55%, 60%, 65%, 70%, 75%, 80%, 85% or 90% of the CpG sites are methylated. Likewise, in some embodiments, the method identifies the target DNA fragment as being from a heart cardiomyocyte when no more than 25%, 30%, 35%, 40%, 45%, or 50% of the CpG sites are unmethylated. In some embodiments, the method identifies the target DNA fragment as not being from a heart cardiomyocyte when no more than 25%, 30%, 35%, 40%, 45%, or 50% of the CpG sites are methylated. In some embodiments, the method identifies the target DNA fragment as not being from a heart cardiomyocyte when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 3130-3147, 3148-3223, 3224-3230, 3231-3254, 3255-3266, 3267-3267 or 3268-3279.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a heart cardiomyocyte of a subject, the method indicates that the subject has abnormal cell death and/or a disease relating to the cell. In some embodiments, the disease or condition is injury, inflammation, or cancer of the heart cardiomyocytes.

In some embodiments, when the amount of cell-free DNA identified as being from a particular type of cell or cells, e.g., heart cardiomyocytes, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., heart cardiomyocytes is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a heart cardiomyocyte, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### C4. Heart Fibroblasts

Also as provided in Table A, some genomic locations are uniformly under-methylated or over-methylated in heart fibroblast cells as compared to all other cell types in the human.

In accordance with one embodiment of the present disclosure, a method is provided for identifying that a biological sample includes DNA from a heart fibroblast cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 33280-3300, 3301-3394, 3395-3396, 3397-3400 or 3401-3407, or selected from SEQ ID NO: 3280-3300 or 3301-3394. In some embodiments, the method then identifies the target DNA fragment as being from a heart fibroblast cell when no more than 40% of the CpG sites are methylated. In some embodiments, the method identifies the target DNA fragment as being from a heart fibroblast cell when no more than 25%, 30%, 35%, 40%, 45%, or 50% of the CpG sites are methylated. Likewise, in some embodiments, the method identifies the target DNA fragment as being from a heart fibroblast cell when at least 50%, 55%, 60%, 65%, 70%, 75%, 80%, 85% or 90% of the CpG sites are unmethylated. In some embodiments, the method identifies the target DNA fragment as not being from a heart fibroblast cell when at least 50%, 55%, 60%, 65%, 70%, 75%, 80%, 85% or 90% of the CpG sites are methylated. In some embodiments, the method identifies the target DNA fragment as not being from a heart fibroblast cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 3408-3414, 3415-3416 or 3417-3432, or selected from SEQ ID NO: 3408-3414. In some embodiments, the method then identifies the target DNA fragment as being from a heart fibroblast cell when 50% or more of the CpG sites are methylated. In some embodiments, the method identifies the target DNA fragment as being from a heart fibroblast cell when at least 55%, 60%, 65%, 70%, 75%, 80%, 85% or 90% of the CpG sites are methylated. Likewise, in some embodiments, the method identifies the target DNA fragment as being from a heart fibroblast cell when no more than 25%, 30%, 35%, 40%, 45%, or 50% of the CpG sites are unmethylated. In some embodiments, the method identifies the target DNA fragment as not being from a heart fibroblast cell when no more than 25%, 30%, 35%, 40%, 45%, or 50% of the CpG sites are methylated. In some embodiments, the method identifies the target DNA fragment as not being from a heart fibroblast cell when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 3280-3300, 3301-3394, 3395-3396, 3397-3400, 3401-3407, 3408-3414, 3415-3416 or 3417-3432.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a heart fibroblast cell of a subject, the method indicates that the subject has abnormal cell death and/or a disease relating to the cell. In some embodiments, the disease or condition is injury, inflammation, or cancer of the heart fibroblast cells.

In some embodiments, when the amount of cell-free DNA identified as being from a particular type of cell or cells, e.g., heart fibroblast cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., heart fibroblast cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a heart fibroblast cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### C5. Vascular Endothelial Cells

Also as provided in Table A, some genomic locations are uniformly under-methylated or over-methylated in vascular endothelial cells as compared to all other cell types in the human.

In accordance with one embodiment of the present disclosure, a method is provided for identifying that a biological sample includes DNA from a vascular endothelial cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 3433-3456,3457-3547, 3548-3550, 3551-3551 or 3552-3559, or selected from SEQ ID NO: 3433-3456 or 3457-3547. In some embodiments, the method then identifies the target DNA fragment as being from a vascular endothelial cell when no more than 40% of the CpG sites are methylated. In some embodiments, the method identifies the target DNA fragment as being from a vascular endothelial cell when no more than 25%, 30%, 35%, 40%, 45%, or 50% of the CpG sites are methylated. Likewise, in some embodiments, the method identifies the target DNA fragment as being from a vascular endothelial cell when at least 50%, 55%, 60%, 65%, 70%, 75%, 80%, 85% or 90% of the CpG sites are unmethylated. In some embodiments, the method identifies the target DNA fragment as not being from a vascular endothelial cell when at least 50%, 55%, 60%, 65%, 70%, 75%, 80%, 85% or 90% of the CpG sites are methylated. In some embodiments, the method identifies the target DNA fragment as not being from a vascular endothelial cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 3560-3579, 3580-3580 or 3581-3584, or selected from SEQ ID NO: 3560-3579. In some embodiments, the method then identifies the target DNA fragment as being from a vascular endothelial cell when 50% or more of the CpG sites are methylated. In some embodiments, the method identifies the target DNA fragment as being from a vascular endothelial cell when at least 55%, 60%, 65%, 70%, 75%, 80%, 85% or 90% of the CpG sites are methylated. Likewise, in some embodiments, the method identifies the target DNA fragment as being from a vascular endothelial cell when no more than 25%, 30%, 35%, 40%, 45%, or 50% of the CpG sites are unmethylated. In some embodiments, the method identifies the target DNA fragment as not being from a vascular endothelial cell when no more than 25%, 30%, 35%, 40%, 45%, or 50% of the CpG sites are methylated. In some embodiments, the method identifies the target DNA fragment as not being from a vascular endothelial cell when at least 50%, 55%, 60%, 65%, 70%, 75%, 80%/o, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 3433-3456, 3457-3547, 3548-3550, 3551-3551, 3552-3559, 3560-3579, 3580-3580 or 3581-3584.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a vascular endothelial cell of a subject, the method indicates that the subject has abnormal cell death and/or a disease relating to the cell. In some embodiments, the disease or condition is injury, inflammation, or cancer of the vascular endothelial cells.

In some embodiments, when the amount of cell-free DNA identified as being from a particular type of cell or cells, e.g., vascular endothelial cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., vascular endothelial cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a vascular endothelial cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### Group 8 Heart Cardiomyocytes & Heart Fibroblasts

Also as provided in Table A, some genomic locations are uniformly under-methylated or over-methylated in a group of cells, namely heart cardiomyocytes & heart fibroblasts, as compared to all other cell types in the human.

In accordance with one embodiment of the present disclosure, a method is provided for identifying that a biological sample includes DNA from a cell selected from heart cardiomyocytes & heart fibroblasts. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 6940-6959, 6960-7045, 7046-7046, 7047-7049, 7050-7053 or 7054-7065, or selected from SEQ ID NO: 6940-6959 or 6960-7045. In some embodiments, the method then identifies the target DNA fragment as being from a cell selected from heart cardiomyocytes & heart fibroblasts when no more than 40% of the CpG sites are methylated. In some embodiments, the method identifies the target DNA fragment as being from a cell selected from heart cardiomyocytes & heart fibroblasts when no more than 25%, 30%, 35%, 40%, 45%, or 50% of the CpG sites are methylated. Likewise, in some embodiments, the method identifies the target DNA fragment as being from a cell selected from heart cardiomyocytes & heart fibroblasts when at least 50%, 55%, 60%, 65%, 70%, 75%, 80%, 85% or 90% of the CpG sites are unmethylated. In some embodiments, the method identifies the target DNA fragment as not being from a cell selected from heart cardiomyocytes & heart fibroblasts when at least 50%, 55%, 60%, 65%, 70%, 75%, 80%, 85% or 90% of the CpG sites are methylated. In some embodiments, the method identifies the target DNA fragment as not being from a cell selected from heart cardiomyocytes & heart fibroblasts when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 7066-7082 or 7083-7090, or selected from SEQ ID NO: 7066-7082. In some embodiments, the method then identifies the target DNA fragment as being from a cell selected from heart cardiomyocytes & heart fibroblasts when 50% or more of the CpG sites are methylated. In some embodiments, the method identifies the target DNA fragment as being from a cell selected from heart cardiomyocytes & heart fibroblasts when at least 55%, 60%, 65%, 70%, 75%, 80%, 85% or 90% of the CpG sites are methylated. Likewise, in some embodiments, the method identifies the target DNA fragment as being from a cell selected from heart cardiomyocytes & heart fibroblasts when no more than 25%, 30%, 35%, 40/o, 45%, or 50% of the CpG sites are unmethylated. In some embodiments, the method identifies the target DNA fragment as not being from a cell selected from heart cardiomyocytes & heart fibroblasts when no more than 25%, 30%, 35%, 40%, 45%, or 50% of the CpG sites are methylated. In some embodiments, the method identifies the target DNA fragment as not being from a cell selected from heart cardiomyocytes & heart fibroblasts when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 6940-6959, 6960-7045, 7046-7046, 7047-7049, 7050-7053, 7054-7065, 7066-7082 or 7083-7090.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a cell selected from heart cardiomyocytes & heart fibroblasts of a subject, the method indicates that the subject has abnormal cell death and/or a disease relating to the cell. In some embodiments, the disease or condition is injury, inflammation, or cancer of a cell selected from heart cardiomyocytes & heart fibroblasts.

In some embodiments, when the amount of cell-free DNA identified as being from a particular type of cell or cells, e.g., cells selected from heart cardiomyocytes & heart fibroblasts, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., cells selected from heart cardiomyocytes & heart fibroblasts is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a cell selected from heart cardiomyocytes & heart fibroblasts, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### Group 9 Lung Alveolar Epithelium & Lung Bronchial Epithelium

Also as provided in Table A, some genomic locations are uniformly under-methylated or over-methylated in a group of cells, namely lung alveolar epithelium & lung bronchial epithelium, as compared to all other cell types in the human.

In accordance with one embodiment of the present disclosure, a method is provided for identifying that a biological sample includes DNA from a cell selected from lung alveolar epithelium & lung bronchial epithelium. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 6227-6243, 6244-6326, 6327-6327, 6328-6329, 6330-6336 or 6337-6352, or selected from SEQ ID NO: 6227-6243 or 6244-6326. In some embodiments, the method then identifies the target DNA fragment as being from a cell selected from lung alveolar epithelium & lung bronchial epithelium when no more than 40% of the CpG sites are methylated. In some embodiments, the method identifies the target DNA fragment as being from a cell selected from lung alveolar epithelium & lung bronchial epithelium when no more than 25%, 30%, 35%, 40%, 45%, or 50% of the CpG sites are methylated. Likewise, in some embodiments, the method identifies the target DNA fragment as being from a cell selected from lung alveolar epithelium & lung bronchial epithelium when at least 50%, 55%, 60%, 65%, 70%, 75%, 80%, 85% or 90% of the CpG sites are unmethylated. In some embodiments, the method identifies the target DNA fragment as not being from a cell selected from lung alveolar epithelium & lung bronchial epithelium when at least 50%, 55%, 60%, 65%, 70%, 75%, 80%, 85% or 90% of the CpG sites are methylated. In some embodiments, the method identifies the target DNA fragment as not being from a cell selected from lung alveolar epithelium & lung bronchial epithelium when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 6353 or 6354-6365, or selected from SEQ ID NO: 6353. In some embodiments, the method then identifies the target DNA fragment as being from a cell selected from lung alveolar epithelium & lung bronchial epithelium when 50% or more of the CpG sites are methylated. In some embodiments, the method identifies the target DNA fragment as being from a cell selected from lung alveolar epithelium & lung bronchial epithelium when at least 55%, 60%, 65%, 70%, 75%, 80%, 85% or 90% of the CpG sites are methylated. Likewise, in some embodiments, the method identifies the target DNA fragment as being from a cell selected from lung alveolar epithelium & lung bronchial epithelium when no more than 25%, 30%, 35%, 40%, 45%, or 50% of the CpG sites are unmethylated. In some embodiments, the method identifies the target DNA fragment as not being from a cell selected from lung alveolar epithelium & lung bronchial epithelium when no more than 25%, 30%, 35%, 40%, 45%, or 50% of the CpG sites are methylated. In some embodiments, the method identifies the target DNA fragment as not being from a cell selected from lung alveolar epithelium & lung bronchial epithelium when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 6227-6243, 6244-6326, 6327-6327, 6328-6329, 6330-6336, 6337-6352, 6353 or 6354-6365.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a cell selected from lung alveolar epithelium & lung bronchial epithelium of a subject, the method indicates that the subject has abnormal cell death and/or a disease relating to the cell. In some embodiments, the disease or condition is injury, inflammation, or cancer of a cell selected from lung alveolar epithelium & lung bronchial epithelium.

In some embodiments, when the amount of cell-free DNA identified as being from a particular type of cell or cells, e.g., cells selected from lung alveolar epithelium & lung bronchial epithelium, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., cells selected from lung alveolar epithelium & lung bronchial epithelium is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a cell selected from lung alveolar epithelium & lung bronchial epithelium, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### D. Hematologic Cells

D1. Blood B cells

Also as provided in Table A, some genomic locations are uniformly under-methylated or over-methylated in blood B cells as compared to all other cell types in the human.

In accordance with one embodiment of the present disclosure, a method is provided for identifying that a biological sample includes DNA from a blood B cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 3585-3607, 3608-3701, 3702-3702, 3703-3704 or 3705-3712, or selected from SEQ ID NO: 3585-3607 or 3608-3701. In some embodiments, the method then identifies the target DNA fragment as being from a blood B cell when no more than 40% of the CpG sites are methylated. In some embodiments, the method identifies the target DNA fragment as being from a blood B cell when no more than 25%, 30%, 35%, 40%, 45%, or 50% of the CpG sites are methylated. Likewise, in some embodiments, the method identifies the target DNA fragment as being from a blood B cell when at least 50%, 55%, 60%, 65%, 70%, 75%, 80%, 85% or 90% of the CpG sites are unmethylated. In some embodiments, the method identifies the target DNA fragment as not being from a blood B cell when at least 50%, 55%, 60%, 65%, 70%, 75%, 80%, 85% or 90% of the CpG sites are methylated. In some embodiments, the method identifies the target DNA fragment as not being from a blood B cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 3713-3733 or 3734-3737, or selected from SEQ ID NO: 3713-3733. In some embodiments, the method then identifies the target DNA fragment as being from a blood B cell when 50% or more of the CpG sites are methylated. In some embodiments, the method identifies the target DNA fragment as being from a blood B cell when at least 55%, 60%, 65%, 70%, 75%, 80%, 85% or 90% of the CpG sites are methylated. Likewise, in some embodiments, the method identifies the target DNA fragment as being from a blood B cell when no more than 25%, 30%, 35%, 40%, 45%, or 50% of the CpG sites are unmethylated. In some embodiments, the method identifies the target DNA fragment as not being from a blood B cell when no more than 25%, 30%, 35%, 40%, 45%, or 50% of the CpG sites are methylated. In some embodiments, the method identifies the target DNA fragment as not being from a blood B cell when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 3585-3607, 3608-3701, 3702-3702, 3703-3704, 3705-3712, 3713-3733 or 3734-3737.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a blood B cell of a subject, the method indicates that the subject has abnormal cell death and/or a disease relating to the cell. In some embodiments, the disease or condition is injury, inflammation, or cancer of the blood B cells. In some embodiments, the disease or condition is an autoimmune disease or infection.

In some embodiments, when the amount of cell-free DNA identified as being from a particular type of cell or cells, e.g., blood B cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., blood B cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a blood B cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

D2. Blood Granulocyles

Also as provided in Table A, some genomic locations are uniformly under-methylated or over-methylated in blood granulocytes as compared to all other cell types in the human.

In accordance with one embodiment of the present disclosure, a method is provided for identifying that a biological sample includes DNA from a blood granulocyte. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 3738-3758, 3759-3849,3850-3851, 3852-3855 or 3856-3862, or selected from SEQ ID NO: 3738-3758 or 3759-3849. In some embodiments, the method then identifies the target DNA fragment as being from a blood granulocyte when no more than 40% of the CpG sites are methylated. In some embodiments, the method identifies the target DNA fragment as being from a blood granulocyte when no more than 25%, 30%, 35%, 40%, 45%, or 50% of the CpG sites are methylated. Likewise, in some embodiments, the method identifies the target DNA fragment as being from a blood granulocyte when at least 50%, 55%, 60%, 65%, 70%, 75%, 80%, 85% or 90% of the CpG sites are unmethylated. In some embodiments, the method identifies the target DNA fragment as not being from a blood granulocyte when at least 50%, 55%, 60%, 65%, 70%, 75%, 80%, 85% or 90% of the CpG sites are methylated. In some embodiments, the method identifies the target DNA fragment as not being from a blood granulocyte when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 3863-3884, 3885-3885 or 3886-3886, or selected from SEQ ID NO: 3863-3884. In some embodiments, the method then identifies the target DNA fragment as being from a blood granulocyte when 50% or more of the CpG sites are methylated. In some embodiments, the method identifies the target DNA fragment as being from a blood granulocyte when at least 55%, 600, 65%, 70%, 75%, 80%, 85% or 90% of the CpG sites are methylated. Likewise, in some embodiments, the method identifies the target DNA fragment as being from a blood granulocyte when no more than 25%, 30%, 16, 35%, 40%, 45%, or 50% of the CpG sites are unmethylated. In some embodiments, the method identifies the target DNA fragment as not being from a blood granulocyte when no more than 25%, 30%, 35%, 40%, 45%, or 50/% of the CpG sites are methylated. In some embodiments, the method identifies the target DNA fragment as not being from a blood granulocyte when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 3738-3758, 3759-3849, 3850-3851, 3852-3855, 3856-3862, 3863-3884, 3885-3885 or 3886-3886.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a blood granulocyte of a subject, the method indicates that the subject has abnormal cell death and/or a disease relating to the cell. In some embodiments, the disease or condition is injury, inflammation, or cancer of the blood granulocytes. In some embodiments, the disease or condition is an autoimmune disease or infection.

In some embodiments, when the amount of cell-free DNA identified as being from a particular type of cell or cells, e.g., blood granulocytes, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., blood granulocytes is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a blood granulocyte, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### D3. Blood Monocytes+Macrophages

Also as provided in Table A, some genomic locations are uniformly under-methylated or over-methylated in blood monocytes or macrophages as compared to all other cell types in the human.

In accordance with one embodiment of the present disclosure, a method is provided for identifying that a biological sample includes DNA from a blood monocyte or macrophage. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 3887-3909,3910-3997, 3998-4000, 4001-4002 or 4003-4012, or selected from SEQ ID NO: 3887-3909 or 3910-3997. In some embodiments, the method then identifies the target DNA fragment as being from a blood monocyte or macrophage when no more than 40% of the CpG sites are methylated. In some embodiments, the method identifies the target DNA fragment as being from a blood monocyte or macrophage when no more than 25%, 30%, 35%, 40%, 45%, or 50% of the CpG sites are methylated. Likewise, in some embodiments, the method identifies the target DNA fragment as being from a blood monocyte or macrophage when at least 50%, 55%, 60%, 65%, 70%, 75%, 80%, 85% or 90% of the CpG sites are unmethylated. In some embodiments, the method identifies the target DNA fragment as not being from a blood monocyte or macrophage when at least 50%, 55%, 60%, 65%, 70%, 75%, 80%, 85% or 90% of the CpG sites are methylated. In some embodiments, the method identifies the target DNA fragment as not being from a blood monocyte or macrophage when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 4013-4036 or 4037, or selected from SEQ ID NO: 40134036. In some embodiments, the method then identifies the target DNA fragment as being from a blood monocyte or macrophage when 50% or more of the CpG sites are methylated. In some embodiments, the method identifies the target DNA fragment as being from a blood monocyte or macrophage when at least 55%, 60%, 65%, 70%, 75%, 80%, 85% or 90% of the CpG sites are methylated. Likewise, in some embodiments, the method identifies the target DNA fragment as being from a blood monocyte or macrophage when no more than 25%, 30%, 35%, 40%, 45%, or 50% of the CpG sites are unmethylated. In some embodiments, the method identifies the target DNA fragment as not being from a blood monocyte or macrophage when no more than 25%, 30%, 35%, 40%, 45%, or 50% of the CpG sites are methylated. In some embodiments, the method identifies the target DNA fragment as not being from a blood monocyte or macrophage when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 3887-3909, 3910-3997, 3998-4000, 4001-4002, 4003-4012, 4013-4036 or 4037.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a blood monocyte or macrophage of a subject, the method indicates that the subject has abnormal cell death and/or a disease relating to the cell. In some embodiments, the disease or condition is injury, inflammation, or cancer of the blood monocytes or macrophages. In some embodiments, the disease or condition is an autoimmune disease or infection.

In some embodiments, when the amount of cell-free DNA identified as being from a particular type of cell or cells, e.g., blood monocytes or macrophages, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., blood monocytes or macrophages is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a blood monocyte or macrophage, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### D4. Blood NK Cells

Also as provided in Table A, some genomic locations are uniformly under-methylated or over-methylated in blood NK cells as compared to all other cell types in the human.

In accordance with one embodiment of the present disclosure, a method is provided for identifying that a biological sample includes DNA from a blood NK cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 4038-4061, 4062-4146, 4147-4148, 4149-4149 or 4150-4162, or selected from SEQ ID NO: 4038-4061 or 4062-4146. In some embodiments, the method then identifies the target DNA fragment as being from a blood NK cell when no more than 40% of the CpG sites are methylated. In some embodiments, the method identifies the target DNA fragment as being from a blood NK cell when no more than 25%, 30%, 35%, 40%, 45%, or 50% of the CpG sites are methylated. Likewise, in some embodiments, the method identifies the target DNA fragment as being from a blood NK cell when at least 50%, 55%, 60%, 65%, 70%, 75%, 80%, 85% or 90% of the CpG sites are unmethylated. In some embodiments, the method identifies the target DNA fragment as not being from a blood NK cell when at least 50%, 55%, 60%, 65%, 70%, 75%, 80%, 85% or 90% of the CpG sites are methylated. In some embodiments, the method identifies the target DNA fragment as not being from a blood NK cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 4163-4184 or 4185-4187, or selected from SEQ ID NO: 4163-4184. In some embodiments, the method then identifies the target DNA fragment as being from a blood NK cell when 50% or more of the CpG sites are methylated. In some embodiments, the method identifies the target DNA fragment as being from a blood NK cell when at least 55%, 60%, 65%, 70%, 75%, 80%, 85% or 90% of the CpG sites are methylated. Likewise, in some embodiments, the method identifies the target DNA fragment as being from a blood NK cell when no more than 25%, 30%, 35%, 40%, 45%, or 50% of the CpG sites are unmethylated. In some embodiments, the method identifies the target DNA fragment as not being from a blood NK cell when no more than 25%, 30%, 35%, 40%, 45%, or 50% of the CpG sites are methylated. In some embodiments, the method identifies the target DNA fragment as not being from a blood NK cell when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 4038-4061, 4062-4146, 4147-4148, 4149-4149, 4150-4162, 4163-4184 or 4185-4187.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a blood NK cell of a subject, the method indicates that the subject has abnormal cell death and/or a disease relating to the cell. In some embodiments, the disease or condition is injury, inflammation, or cancer of the blood NK cells. In some embodiments, the disease or condition is an autoimmune disease or infection.

In some embodiments, when the amount of cell-free DNA identified as being from a particular type of cell or cells, e.g., blood NK cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., blood NK cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a blood NK cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### D5. Blood T Cells

Also as provided in Table A, some genomic locations are uniformly under-methylated or over-methylated in blood T cells as compared to all other cell types in the human.

In accordance with one embodiment of the present disclosure, a method is provided for identifying that a biological sample includes DNA from a blood T cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 4188-4205, 4206-4274, 4275-4275, 4276-4276, 4277-4282 or 4283-4312, or selected from SEQ 1D NO: 4188-4205 or 4206-4274. In some embodiments, the method then identifies the target DNA fragment as being from a blood T cell when no more than 40% of the CpG sites are methylated. In some embodiments, the method identifies the target DNA fragment as being from a blood T cell when no more than 25%, 30%, 35%, 40%, 45%, or 50% of the CpG sites are methylated. Likewise, in some embodiments, the method identifies the target DNA fragment as being from a blood T cell when at least 50%, 55%, 60%, 65%, 70%, 75%, 80%, 85% or 90% of the CpG sites are unmethylated. In some embodiments, the method identifies the target DNA fragment as not being from a blood T cell when at least 50%, 55%, 60%, 65%, 70%, 75%, 80%, 85% or 90% of the CpG sites are methylated. In some embodiments, the method identifies the target DNA fragment as not being from a blood T cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 4313-4322, 4323-4323 or 4324-4337, or selected from SEQ ID NO: 4313-4322. In some embodiments, the method then identifies the target DNA fragment as being from a blood T cell when 50% or more of the CpG sites are methylated. In some embodiments, the method identifies the target DNA fragment as being from a blood T cell when at least 55%, 60%, 65%, 70%, 75%, 80%, 85% or 90% of the CpG sites are methylated. Likewise, in some embodiments, the method identifies the target DNA fragment as being from a blood T cell when no more than 25%, 30%, 35%, 40%, 45%, or 50% of the CpG sites are unmethylated. In some embodiments, the method identifies the target DNA fragment as not being from a blood T cell when no more than 25%, 30%, 35%, 40%, 45%, or 50% of the CpG sites are methylated. In some embodiments, the method identifies the target DNA fragment as not being from a blood T cell when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 4188-4205, 4206-4274, 4275-4275, 4276-4276, 4277-4282, 4283-4312, 4313-4322, 4323-4323 or 4324-4337.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a blood T cell of a subject, the method indicates that the subject has abnormal cell death and/or a disease relating to the cell. In some embodiments, the disease or condition is injury, inflammation, or cancer of the blood T cells. In some embodiments, the disease or condition is an autoimmune disease or infection.

In some embodiments, when the amount of cell-free DNA identified as being from a particular type of cell or cells, e.g., blood T cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., blood T cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a blood T cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### D6. Erythrocyte Progenitor Cells

Also as provided in Table A, some genomic locations are uniformly under-methylated or over-methylated in erythrocyte progenitor cells as compared to all other cell types in the human.

In accordance with one embodiment of the present disclosure, a method is provided for identifying that a biological sample includes DNA from an erythrocyte progenitor cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 4338-4361,4362-4449, 4450-4453, 4454-4454 or 4455-4464, or selected from SEQ 1D NO: 4338-4361 or 4362-4449. In some embodiments, the method then identifies the target DNA fragment as being from an erythrocyte progenitor cell when no more than 40% of the CpG sites are methylated. In some embodiments, the method identifies the target DNA fragment as being from an erythrocyte progenitor cell when no more than 25%, 30%, 35%, 40%, 45%, or 50% of the CpG sites are methylated. Likewise, in some embodiments, the method identifies the target DNA fragment as being from an erythrocyte progenitor cell when at least 50%, 55%, 60%, 65%, 70%, 75%, 80%, 85% or 90% of the CpG sites are unmethylated. In some embodiments, the method identifies the target DNA fragment as not being from an erythrocyte progenitor cell when at least 50%, 55%, 60%, 65%, 70%, 75%, 80%, 85% or 90% of the CpG sites are methylated. In some embodiments, the method identifies the target DNA fragment as not being from an erythrocyte progenitor cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 4465-4470. In some embodiments, the method then identifies the target DNA fragment as being from an erythrocyte progenitor cell when 50% or more of the CpG sites are methylated. In some embodiments, the method identifies the target DNA fragment as being from an erythrocyte progenitor cell when at least 55%, 60%, 65%, 70%, 75%, 80%, 85% or 90% of the CpG sites are methylated. Likewise, in some embodiments, the method identifies the target DNA fragment as being from an erythrocyte progenitor cell when no more than 25%, 30%, 35%, 40%, 45%, or 50% of the CpG sites are unmethylated. In some embodiments, the method identifies the target DNA fragment as not being from an erythrocyte progenitor cell when no more than 25%, 30%, 35%, 40%, 45%, or 50% of the CpG sites are methylated. In some embodiments, the method identifies the target DNA fragment as not being from an erythrocyte progenitor cell when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 4338-4361, 4362-4449, 4450-4453, 4454-4454, 4455-4464, 4465-4470.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from an erythrocyte progenitor cell of a subject, the method indicates that the subject has abnormal cell death and/or a disease relating to the cell. In some embodiments, the disease or condition is injury, inflammation, or cancer of the erythrocyte progenitor cells. In some embodiments, the disease or condition is an autoimmune disease or infection.

In some embodiments, when the amount of cell-free DNA identified as being from a particular type of cell or cells, e.g., erythrocyte progenitor cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., erythrocyte progenitor cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as an erythrocyte progenitor cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### E. Dermal-Skeleto-Muscular Cells

E1. Epidermal keratinocytes

Also as provided in Table A, some genomic locations are uniformly under-methylated or over-methylated in epidermal keratinocytes as compared to all other cell types in the human.

In accordance with one embodiment of the present disclosure, a method is provided for identifying that a biological sample includes DNA from an epidermal keratinocyte. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 4471-4492, 4493-4573, 4574-4574, 4575-4577, 4578-4579 or 4580-4595, or selected from SEQ ID NO: 4471-4492 or 4493-4573. In some embodiments, the method then identifies the target DNA fragment as being from an epidermal keratinocyte when no more than 40% of the CpG sites are methylated. In some embodiments, the method identifies the target DNA fragment as being from an epidermal keratinocyte when no more than 25%, 30%, 35%, 40%, 45%, or 50% of the CpG sites are methylated. Likewise, in some embodiments, the method identifies the target DNA fragment as being from an epidermal keratinocyte when at least 50%, 55%, 60%, 65%, 70%, 75%, 80%, 85% or 90% of the CpG sites are unmethylated. In some embodiments, the method identifies the target DNA fragment as not being from an epidermal keratinocyte when at least 50%, 55%, 600, 65%, 70%, 75%, 80%, 85% or 90% of the CpG sites are methylated. In some embodiments, the method identifies the target DNA fragment as not being from an epidermal keratinocyte when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 4596-4598, 4599-4599 or 4600-4618, or preferably SEQ ID NO: 4596-4598. In some embodiments, the method then identifies the target DNA fragment as being from an epidermal keratinocyte when 50% or more of the CpG sites are methylated. In some embodiments, the method identifies the target DNA fragment as being from an epidermal keratinocyte when at least 55%, 60%, 65%, 70%, 75%, 80%, 85% or 90% of the CpG sites are methylated. Likewise, in some embodiments, the method identifies the target DNA fragment as being from an epidermal keratinocyte when no more than 25%, 30%, 35%, 40%, 45%, or 50% of the CpG sites are unmethylated. In some embodiments, the method identifies the target DNA fragment as not being from an epidermal keratinocyte when no more than 25%, 30%, 35%, 40%, 45%, or 50% of the CpG sites are methylated. In some embodiments, the method identifies the target DNA fragment as not being from an epidermal keratinocyte when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 4471-4492, 4493-4573, 4574-4574, 4575-4577, 4578-4579, 4580-4595, 4596-4598, 4599-4599 or 4600-4618.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from an epidermal keratinocyte of a subject, the method indicates that the subject has abnormal cell death and/or a disease relating to the cell. In some embodiments, the disease or condition is injury, inflammation, or cancer of the epidermal keratinocytes.

In some embodiments, when the amount of cell-free DNA identified as being from a particular type of cell or cells, e.g., epidermal keratinocytes, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., epidermal keratinocytes is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as an epidermal keratinocyte, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### E2. Dermal Fibroblasts

Also as provided in Table A, some genomic locations are uniformly under-methylated or over-methylated in dermal fibroblast cells as compared to all other cell types in the human.

In accordance with one embodiment of the present disclosure, a method is provided for identifying that a biological sample includes DNA from a dermal fibroblast cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 4619-4641,4642-4719, 4720, 4721-4727, 4728 or 4729-4741, or selected from SEQ ID NO: 4619-4641 or 46424719. In some embodiments, the method then identifies the target DNA fragment as being from a dermal fibroblast cell when no more than 40% of the CpG sites are methylated. In some embodiments, the method identifies the target DNA fragment as being from a dermal fibroblast cell when no more than 25%, 30%, 35%, 40%, 45%, or 50% of the CpG sites are methylated. Likewise, in some embodiments, the method identifies the target DNA fragment as being from a dermal fibroblast cell when at least 50%, 55%, 60%, 65%, 70%, 75%, 80%, 85% or 90% of the CpG sites are unmethylated. In some embodiments, the method identifies the target DNA fragment as not being from a dermal fibroblast cell when at least 50%, 55%, 60%, 65%, 70%, 75%, 80%, 85% or 90% of the CpG sites are methylated. In some embodiments, the method identifies the target DNA fragment as not being from a dermal fibroblast cell when no more than 25%, 30%, 35%, 40%, 16, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 4742-4747, 4748 or 4749-4766, or selected from SEQ ID NO: 4742-4747. In some embodiments, the method then identifies the target DNA fragment as being from a dermal fibroblast cell when 50% or more of the CpG sites are methylated. In some embodiments, the method identifies the target DNA fragment as being from a dermal fibroblast cell when at least 55%, 60%, 65%, 70%, 75%, 80%, 85% or 90% of the CpG sites are methylated. Likewise, in some embodiments, the method identifies the target DNA fragment as being from a dermal fibroblast cell when no more than 25%, 30%, 35%, 40%, 45%, or 500/% of the CpG sites are unmethylated. In some embodiments, the method identifies the target DNA fragment as not being from a dermal fibroblast cell when no more than 25%, 30%, 35%, 40%, 45%, or 50% of the CpG sites are methylated. In some embodiments, the method identifies the target DNA fragment as not being from a dermal fibroblast cell when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 4619-4641, 46424719, 4720, 47214727, 4728, 4729-4741, 47424747, 4748 or 4749-4766.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a dermal fibroblast cell of a subject, the method indicates that the subject has abnormal cell death and/or a disease relating to the cell. In some embodiments, the disease or condition is injury, inflammation, or cancer of the dermal fibroblast cells.

In some embodiments, when the amount of cell-free DNA identified as being from a particular type of cell or cells, e.g., dermal fibroblast cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., dermal fibroblast cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a dermal fibroblast cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### E3. Osteoblasts

Also as provided in Table A, some genomic locations are uniformly under-methylated or over-methylated in osteoblast cells as compared to all other cell types in the human.

In accordance with one embodiment of the present disclosure, a method is provided for identifying that a biological sample includes DNA from an osteoblast cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 47674783, 4784-4869, 4870-4872, 4873-4877, 4878-4882 or 4883-4891, or selected from SEQ ID NO: 4767-4783 or 4784-4869. In some embodiments, the method then identifies the target DNA fragment as being from an osteoblast cell when no more than 40% of the CpG sites are methylated. In some embodiments, the method identifies the target DNA fragment as being from an osteoblast cell when no more than 25%, 30%, 35%, 40%, 45%, or 50% of the CpG sites are methylated. Likewise, in some embodiments, the method identifies the target DNA fragment as being from an osteoblast cell when at least 50%, 55%, 60%, 65%, 70%, 75%, 80%, 85% or 90% of the CpG sites are unmethylated. In some embodiments, the method identifies the target DNA fragment as not being from an osteoblast cell when at least 50%, 55%, 60%, 65%, 70%, 75%, 80%, 85% or 90% of the CpG sites are methylated. In some embodiments, the method identifies the target DNA fragment as not being from an osteoblast cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 4892-4897 or 4898-4916, or selected from SEQ ID NO: 4892-4897. In some embodiments, the method then identifies the target DNA fragment as being from an osteoblast cell when 50% or more of the CpG sites are methylated. In some embodiments, the method identifies the target DNA fragment as being from an osteoblast cell when at least 55%, 60%, 65%, 70%, 75%, 80%, 85% or 90% of the CpG sites are methylated. Likewise, in some embodiments, the method identifies the target DNA fragment as being from an osteoblast cell when no more than 25%, 30%, 35%, 40%, 45%, or 50% of the CpG sites are unmethylated. In some embodiments, the method identifies the target DNA fragment as not being from an osteoblast cell when no more than 25%, 30%, 35%, 40%, 45%, or 50% of the CpG sites are methylated. In some embodiments, the method identifies the target DNA fragment as not being from an osteoblast cell when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 4767-4783, 4784-4869, 4870-4872, 4873-4877, 4878-4882, 4883-4891, 4892-4897 or 4898-4916.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from an osteoblast cell of a subject, the method indicates that the subject has abnormal cell death and/or a disease relating to the cell. In some embodiments, the disease or condition is injury, inflammation, or cancer of the osteoblast cells.

In some embodiments, when the amount of cell-free DNA identified as being from a particular type of cell or cells, e.g., osteoblast cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., osteoblast cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as an osteoblast cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### E4. Skeletal Muscle Cells

Also as provided in Table A, some genomic locations are uniformly under-methylated or over-methylated in skeletal muscle cells as compared to all other cell types in the human.

In accordance with one embodiment of the present disclosure, a method is provided for identifying that a biological sample includes DNA from a skeletal muscle cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 4917-4937, 4938-5016, 5017-5017, 5018-5023, 5024-5026 or 5027-5040, or selected from SEQ ID NO: 4917-4937 or 4938-5016. In some embodiments, the method then identifies the target DNA fragment as being from a skeletal muscle cell when no more than 40% of the CpG sites are methylated. In some embodiments, the method identifies the target DNA fragment as being from a skeletal muscle cell when no more than 25%, 30%, 35%, 40%, 45%, or 50% of the CpG sites are methylated. Likewise, in some embodiments, the method identifies the target DNA fragment as being from a skeletal muscle cell when at least 50%, 55%, 60%, 65%, 70%, 75%, 80%, 85% or 90% of the CpG sites are unmethylated. In some embodiments, the method identifies the target DNA fragment as not being from a skeletal muscle cell when at least 50%, 55%, 60%, 65%, 70%, 75%, 80%, 85% or 90% of the CpG sites are methylated. In some embodiments, the method identifies the target DNA fragment as not being from a skeletal muscle cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 5041-5043, 5044-5045 or 5046-5064, or selected from SEQ ID NO: 5041-5043. In some embodiments, the method then identifies the target DNA fragment as being from a skeletal muscle cell when 50% or more of the CpG sites are methylated. In some embodiments, the method identifies the target DNA fragment as being from a skeletal muscle cell when at least 55%, 60%, 65%, 70%, 75%, 80%, 85% or 90% of the CpG sites are methylated. Likewise, in some embodiments, the method identifies the target DNA fragment as being from a skeletal muscle cell when no more than 25%, 30%, 35%, 40%, 45%, or 50% of the CpG sites are unmethylated. In some embodiments, the method identifies the target DNA fragment as not being from a skeletal muscle cell when no more than 25%, 30%, 35%, 40%, 45%, or 50% of the CpG sites are methylated. In some embodiments, the method identifies the target DNA fragment as not being from a skeletal muscle cell when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 4917-4937, 4938-5016, 5017-5017, 5018-5023, 5024-5026, 5027-5040, 5041-5043, 5044-5045 or 5046-5064.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a skeletal muscle cell of a subject, the method indicates that the subject has abnormal cell death and/or a disease relating to the cell. In some embodiments, the disease or condition is injury, inflammation, or cancer of the skeletal muscle cells.

In some embodiments, when the amount of cell-free DNA identified as being from a particular type of cell or cells, e.g., skeletal muscle cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., skeletal muscle cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a skeletal muscle cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### E5. Smooth Muscle Cells

Also as provided in Table A, some genomic locations are uniformly under-methylated or over-methylated in smooth muscle cells as compared to all other cell types in the human.

In accordance with one embodiment of the present disclosure, a method is provided for identifying that a biological sample includes DNA from a smooth muscle cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 5065-5086, 5087-5178, 5179-5179, 5180-5181, 5182-5183 or 5184-5191, or selected from SEQ ID NO: 5065-5086 or 5087-5178. In some embodiments, the method then identifies the target DNA fragment as being from a smooth muscle cell when no more than 40% of the CpG sites are methylated. In some embodiments, the method identifies the target DNA fragment as being from a smooth muscle cell when no more than 25%, 30%, 35%, 40%, 45%, or 50% of the CpG sites are methylated. Likewise, in some embodiments, the method identifies the target DNA fragment as being from a smooth muscle cell when at least 50%, 55%, 60%, 65%, 70%, 75%, 80%, 85% or 90% of the CpG sites are unmethylated. In some embodiments, the method identifies the target DNA fragment as not being from a smooth muscle cell when at least 50%, 55%, 60%, 65%, 70%, 75%, 80%, 85% or 90% of the CpG sites are methylated. In some embodiments, the method identifies the target DNA fragment as not being from a smooth muscle cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 5192-5204, 5205-5207 or 5208-5216, or selected from SEQ ID NO: 5192-5204. In some embodiments, the method then identifies the target DNA fragment as being from a smooth muscle cell when 50% c or more of the CpG sites are methylated. In some embodiments, the method identifies the target DNA fragment as being from a smooth muscle cell when at least 55%, 60%, 65%, 70%, 75%, 80%, 85% or 90% of the CpG sites are methylated. Likewise, in some embodiments, the method identifies the target DNA fragment as being from a smooth muscle cell when no more than 25%, 30%, 35%, 40%, 45%, or 50% of the CpG sites are unmethylated. In some embodiments, the method identifies the target DNA fragment as not being from a smooth muscle cell when no more than 25%, 30%, 35%, 40%, 45%, or 50% of the CpG sites are methylated. In some embodiments, the method identifies the target DNA fragment as not being from a smooth muscle cell when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 5065-5086, 5087-5178, 5179-5179, 5180-5181, 5182-5183, 5184-5191, 5192-5204, 5205-5207 or 5208-5216.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a smooth muscle cell of a subject, the method indicates that the subject has abnormal cell death and/or a disease relating to the cell. In some embodiments, the disease or condition is injury, inflammation, or cancer of the smooth muscle cells.

In some embodiments, when the amount of cell-free DNA identified as being from a particular type of cell or cells, e.g., smooth muscle cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., smooth muscle cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a smooth muscle cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### Group 10 Heart Cardiomyocytes & Skeletal Muscle Cell & Smooth Muscle Cells

Also as provided in Table A, some genomic locations are uniformly under-methylated or over-methylated in a group of cells, namely heart cardiomyocytes & skeletal muscle cell & smooth muscle cells, as compared to all other cell types in the human.

In accordance with one embodiment of the present disclosure, a method is provided for identifying that a biological sample includes DNA from a cell selected from heart cardiomyocytes & skeletal muscle cell & smooth muscle cells. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 6899-6906, 6907 or 6908-6909, or selected from SEQ ID NO: 6899-6906 or 6907. In some embodiments, the method then identifies the target DNA fragment as being from a cell selected from heart cardiomyocytes & skeletal muscle cell & smooth muscle cells when no more than 40% of the CpG sites are methylated. In some embodiments, the method identifies the target DNA fragment as being from a cell selected from heart cardiomyocytes & skeletal muscle cell & smooth muscle cells when no more than 25%, 30%, 35%, 40%, 45%, or 50% of the CpG sites are methylated. Likewise, in some embodiments, the method identifies the target DNA fragment as being from a cell selected from heart cardiomyocytes & skeletal muscle cell & smooth muscle cells when at least 50%, 55%, 60%, 65%, 70%, 75%, 80%, 85% or 90% of the CpG sites are unmethylated. In some embodiments, the method identifies the target DNA fragment as not being from a cell selected from heart cardiomyocytes & skeletal muscle cell & smooth muscle cells when at least 50%, 55%, 60%, 65%, 70%, 75%, 80%, 85% or 90% of the CpG sites are methylated. In some embodiments, the method identifies the target DNA fragment as not being from a cell selected from heart cardiomyocytes & skeletal muscle cell & smooth muscle cells when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 6910-6911. In some embodiments, the method then identifies the target DNA fragment as being from a cell selected from heart cardiomyocytes & skeletal muscle cell & smooth muscle cells when 50% or more of the CpG sites are methylated. In some embodiments, the method identifies the target DNA fragment as being from a cell selected from heart cardiomyocytes & skeletal muscle cell & smooth muscle cells when at least 55%, 60%, 65%, 70%, 75%, 80%, 85% or 90% of the CpG sites are methylated. Likewise, in some embodiments, the method identifies the target DNA fragment as being from a cell selected from heart cardiomyocytes & skeletal muscle cell & smooth muscle cells when no more than 25%, 30%, 35%, 40%, 45%, or 50% of the CpG sites are unmethylated. In some embodiments, the method identifies the target DNA fragment as not being from a cell selected from heart cardiomyocytes & skeletal muscle cell & smooth muscle cells when no more than 25%, 30%, 35%, 40%, 45%, or 50% of the CpG sites are methylated. In some embodiments, the method identifies the target DNA fragment as not being from a cell selected from heart cardiomyocytes & skeletal muscle cell & smooth muscle cells when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 6899-6906, 6907, 6908-6909, 6910-6911.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a cell selected from heart cardiomyocytes & skeletal muscle cell & smooth muscle cells of a subject, the method indicates that the subject has abnormal cell death and/or a disease relating to the cell. In some embodiments, the disease or condition is injury, inflammation, or cancer of a cell selected from heart cardiomyocytes & skeletal muscle cell & smooth muscle cells.

In some embodiments, when the amount of cell-free DNA identified as being from a particular type of cell or cells, e.g., cells selected from heart cardiomyocytes & skeletal muscle cell & smooth muscle cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., cells selected from heart cardiomyocytes & skeletal muscle cell & smooth muscle cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a cell selected from heart cardiomyocytes & skeletal muscle cell & smooth muscle cells, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### Group 11 Skeletal Muscle Cells & Smooth Muscle Cells

Also as provided in Table A, some genomic locations are uniformly under-methylated or over-methylated in a group of cells, namely skeletal muscle cells & smooth muscle cells, as compared to all other cell types in the human.

In accordance with one embodiment of the present disclosure, a method is provided for identifying that a biological sample includes DNA from a cell selected from skeletal muscle cells & smooth muscle cells. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 6912-6929, 6930-6930 or 6931-6931, or selected from SEQ ID NO: 6912-6929 or 6930-6930. In some embodiments, the method then identifies the target DNA fragment as being from a cell selected from skeletal muscle cells & smooth muscle cells when no more than 40% of the CpG sites are methylated. In some embodiments, the method identifies the target DNA fragment as being from a cell selected from skeletal muscle cells & smooth muscle cells when no more than 25%, 30%, 35%, 40%, 45%, or 50% of the CpG sites are methylated. Likewise, in some embodiments, the method identifies the target DNA fragment as being from a cell selected from skeletal muscle cells & smooth muscle cells when at least 50%, 55%, 60%, 65%, 70%, 75%, 80%, 85% or 90% of the CpG sites are unmethylated. In some embodiments, the method identifies the target DNA fragment as not being from a cell selected from skeletal muscle cells & smooth muscle cells when at least 50%, 55%, 60%, 65%, 70%, 75%, 80%, 85% or 90% of the CpG sites are methylated. In some embodiments, the method identifies the target DNA fragment as not being from a cell selected from skeletal muscle cells & smooth muscle cells when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 6932-6936 or 6937-6939, or selected from SEQ ID NO: 6932-6936. In some embodiments, the method then identifies the target DNA fragment as being from a cell selected from skeletal muscle cells & smooth muscle cells when 50% or more of the CpG sites are methylated. In some embodiments, the method identifies the target DNA fragment as being from a cell selected from skeletal muscle cells & smooth muscle cells when at least 55%, 60%, 65%, 70%, 75%, 80%, 85% or 90% of the CpG sites are methylated. Likewise, in some embodiments, the method identifies the target DNA fragment as being from a cell selected from skeletal muscle cells & smooth muscle cells when no more than 25%, 30%, 35%, 40%, 45%, or 50% of the CpG sites are unmethylated. In some embodiments, the method identifies the target DNA fragment as not being from a cell selected from skeletal muscle cells & smooth muscle cells when no more than 25%, 30%, 35%, 40%, 45%, or 50% of the CpG sites are methylated. In some embodiments, the method identifies the target DNA fragment as not being from a cell selected from skeletal muscle cells & smooth muscle cells when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 6912-6929, 6930-6930, 6931-6931, 6932-6936 or 6937-6939.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a cell selected from skeletal muscle cells & smooth muscle cells of a subject, the method indicates that the subject has abnormal cell death and/or a disease relating to the cell. In some embodiments, the disease or condition is injury, inflammation, or cancer of a cell selected from skeletal muscle cells & smooth muscle cells.

In some embodiments, when the amount of cell-free DNA identified as being from a particular type of cell or cells, e.g., cells selected from skeletal muscle cells & smooth muscle cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., cells selected from skeletal muscle cells & smooth muscle cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a cell selected from skeletal muscle cells & smooth muscle cells, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### F. Neural Cells and Others

### F1. Thyroid Epithelium

Also as provided in Table A, some genomic locations are uniformly under-methylated or over-methylated in thyroid epithelial cells as compared to all other cell types in the human.

In accordance with one embodiment of the present disclosure, a method is provided for identifying that a biological sample includes DNA from a thyroid epithelial cell. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 5217-5230, 5231-5284, 5285, 5286-5296 or 5297-5343, or selected from SEQ ID NO: 5217-5230 or 5231-5284. In some embodiments, the method then identifies the target DNA fragment as being from a thyroid epithelial cell when no more than 40% of the CpG sites are methylated. In some embodiments, the method identifies the target DNA fragment as being from a thyroid epithelial cell when no more than 25%, 30%, 35%, 40%, 45%, or 50% of the CpG sites are methylated. Likewise, in some embodiments, the method identifies the target DNA fragment as being from a thyroid epithelial cell when at least 50%, 55%, 60%, 65%, 70%, 75%, 80%, 85% or 90% of the CpG sites are unmethylated. In some embodiments, the method identifies the target DNA fragment as not being from a thyroid epithelial cell when at least 50%, 55%, 60%, 65%, 70%, 75%, 80%, 85% or 90% of the CpG sites are methylated. In some embodiments, the method identifies the target DNA fragment as not being from a thyroid epithelial cell when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 5344-5358, 5359 or 5360-5368, or selected from SEQ ID NO: 5344-5358. In some embodiments, the method then identifies the target DNA fragment as being from a thyroid epithelial cell when 50% or more of the CpG sites are methylated. In some embodiments, the method identifies the target DNA fragment as being from a thyroid epithelial cell when at least 55%, 60%, 65%, 70%, 75%, 80%, 16, 85% or 90% of the CpG sites are methylated. Likewise, in some embodiments, the method identifies the target DNA fragment as being from a thyroid epithelial cell when no more than 25%, 30%, 35%, 40%, 45%, or 50% of the CpG sites are unmethylated. In some embodiments, the method identifies the target DNA fragment as not being from a thyroid epithelial cell when no more than 25%, 30%, 35%, 40%, 45%, or 50% of the CpG sites are methylated. In some embodiments, the method identifies the target DNA fragment as not being from a thyroid epithelial cell when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 5217-5230, 5231-5284, 5285, 5286-5296, 5297-5343, 5344-5358, 5359 or 5360-5368.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a thyroid epithelial cell of a subject, the method indicates that the subject has abnormal cell death and/or a disease relating to the cell. In some embodiments, the disease or condition is injury, inflammation, or cancer of the thyroid epithelium.

In some embodiments, when the amount of cell-free DNA identified as being from a particular type of cell or cells, e.g., thyroid epithelial cells, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., thyroid epithelial cells is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a thyroid epithelial cell, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### F2. Adipocytes

Also as provided in Table A, some genomic locations are uniformly under-methylated or over-methylated in adipocytes as compared to all other cell types in the human.

In accordance with one embodiment of the present disclosure, a method is provided for identifying that a biological sample includes DNA from an adipocyte. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 5369-5389,5390-5445, 5446, 5447-5449 or 5450-5453, or selected from SEQ ID NO: 5369-5389 or 5390-5445. In some embodiments, the method then identifies the target DNA fragment as being from an adipocyte when no more than 40% of the CpG sites are methylated. In some embodiments, the method identifies the target DNA fragment as being from an adipocyte when no more than 25%, 30%, 35%, 40%, 45%, or 50% of the CpG sites are methylated. Likewise, in some embodiments, the method identifies the target DNA fragment as being from an adipocyte when at least 50%, 55%, 60%, 65%, 70%, 75%, 80%, 85% or 90% of the CpG sites are unmethylated. In some embodiments, the method identifies the target DNA fragment as not being from an adipocyte when at least 50%, 55%, 60%, 65%, 70%, 75%, 80%, 85% or 90% of the CpG sites are methylated. In some embodiments, the method identifies the target DNA fragment as not being from an adipocyte when no more than 25%, 30%, 35%, 40/o, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 5454-5463, 5464 or 5465-5470, or selected from SEQ ID NO: 5454-5463. In some embodiments, the method then identifies the target DNA fragment as being from an adipocyte when 50% or more of the CpG sites are methylated. In some embodiments, the method identifies the target DNA fragment as being from an adipocyte when at least 55%, 60%, 65%, 70%, 75%, 80%, 85% or 90% of the CpG sites are methylated. Likewise, in some embodiments, the method identifies the target DNA fragment as being from an adipocyte when no more than 25%, 30%, 35%, 40%, 45%, or 50% of the CpG sites are unmethylated. In some embodiments, the method identifies the target DNA fragment as not being from an adipocyte when no more than 25%, 30%, 35%, 40%, 45%, or 50% of the CpG sites are methylated. In some embodiments, the method identifies the target DNA fragment as not being from an adipocyte when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 5369-5389, 5390-5445, 5446-5446, 5447-5449, 5450-5453, 5454-5463, 5464 or 5465-5470.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from an adipocyte of a subject, the method indicates that the subject has abnormal cell death and/or a disease relating to the cell. In some embodiments, the disease or condition is injury, inflammation, or cancer of the adipocytes.

In some embodiments, when the amount of cell-free DNA identified as being from a particular type of cell or cells, e.g., adipocytes, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., adipocytes is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as an adipocyte, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### F3. Neuron CNS

Also as provided in Table A, some genomic locations are uniformly under-methylated or over-methylated in neurons as compared to all other cell types in the human.

In accordance with one embodiment of the present disclosure, a method is provided for identifying that a biological sample includes DNA from a neuron. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 5471-5488, 5489-5556, 5557-5559, 5560-5566 or 5567-5594, or selected from SEQ ID NO: 5471-5488 or 5489-5556. In some embodiments, the method then identifies the target DNA fragment as being from a neuron when no more than 40% of the CpG sites are methylated. In some embodiments, the method identifies the target DNA fragment as being from a neuron when no more than 25%, 30%, 35%, 40%, 45%, or 50% of the CpG sites are methylated. Likewise, in some embodiments, the method identifies the target DNA fragment as being from a neuron when at least 50%, 55%, 600%, 65%, 70%, 75%, 80%, 85% or 90% of the CpG sites are unmethylated. In some embodiments, the method identifies the target DNA fragment as not being from a neuron when at least 50%, 55%, 60%, 65%, 70%, 75%, 80%, 85% or 90% of the CpG sites are methylated. In some embodiments, the method identifies the target DNA fragment as not being from a neuron when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 5595-5613 or 5614-5619, or selected from SEQ ID NO: 5595-5613. In some embodiments, the method then identifies the target DNA fragment as being from a neuron when 50% or more of the CpG sites are methylated. In some embodiments, the method identifies the target DNA fragment as being from a neuron when at least 55%, 60%, 65%, 70%, 75%, 80%, 85% or 90% of the CpG sites are methylated. Likewise, in some embodiments, the method identifies the target DNA fragment as being from a neuron when no more than 25%, 30%, 35%, 40%, 45%, or 50% of the CpG sites are unmethylated. In some embodiments, the method identifies the target DNA fragment as not being from a neuron when no more than 25%, 30%, 35%, 40%, 45%, or 50% of the CpG sites are methylated. In some embodiments, the method identifies the target DNA fragment as not being from a neuron when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 5471-5488, 5489-5556, 5557-5559, 5560-5566, 5567-5594, 5595-5613 or 5614-5619.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a neuron of a subject, the method indicates that the subject has abnormal cell death and/or a disease relating to the cell. In some embodiments, the disease or condition is injury, inflammation, or cancer of the neurons. In some embodiments, the disease or condition is a neurodegenerative disorder, such as amyotrophic lateral sclerosis, multiple sclerosis, Parkinson's disease, Alzheimer's disease, Huntington's disease, and prion diseases.

In some embodiments, when the amount of cell-free DNA identified as being from a particular type of cell or cells, e.g., neurons, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., neurons is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a neuron, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

### F4. Oligodendrocytes

Also as provided in Table A, some genomic locations are uniformly under-methylated or over-methylated in oligodendrocytes as compared to all other cell types in the human.

In accordance with one embodiment of the present disclosure, a method is provided for identifying that a biological sample includes DNA from an oligodendrocyte. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 5620-5649,5650-5721, 5722-5724, 5725-5744 or 5745-5771, or selected from SEQ ID NO: 5620-5649 or 5650-5721. In some embodiments, the method then identifies the target DNA fragment as being from an oligodendrocyte when no more than 40% of the CpG sites are methylated. In some embodiments, the method identifies the target DNA fragment as being from an oligodendrocyte when no more than 25%, 30%, 35%, 40%, 45%, or 50% of the CpG sites are methylated. Likewise, in some embodiments, the method identifies the target DNA fragment as being from an oligodendrocyte when at least 50%, 55%, 60%, 65%, 70%, 75%, 80%, 85% or 90% of the CpG sites are unmethylated. In some embodiments, the method identifies the target DNA fragment as not being from an oligodendrocyte when at least 50%, 55%, 60%, 65%, 70%, 75%, 80%, 85% or 90% of the CpG sites are methylated. In some embodiments, the method identifies the target DNA fragment as not being from an oligodendrocyte when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 5772-5782, 5783-5783 or 5784-5796, or selected from SEQ ID NO: 5772-5782. In some embodiments, the method then identifies the target DNA fragment as being from an oligodendrocyte when 50% or more of the CpG sites are methylated. In some embodiments, the method identifies the target DNA fragment as being from an oligodendrocyte when at least 55%, 60%, 65%, 70%, 75%, 80%, 85% or 90% of the CpG sites are methylated. Likewise, in some embodiments, the method identifies the target DNA fragment as being from an oligodendrocyte when no more than 25%, 30%, 35%, 40%, 45%, or 50% of the CpG sites are unmethylated. In some embodiments, the method identifies the target DNA fragment as not being from an oligodendrocyte when no more than 25%, 30%, 35%, 40%, 16, 45%, or 50% of the CpG sites are methylated. In some embodiments, the method identifies the target DNA fragment as not being from an oligodendrocyte when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 5620-5649, 5650-5721, 5722-5724, 5725-5744, 5745-5771, 5772-5782, 5783-5783 or 5784-5796.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from an oligodendrocyte of a subject, the method indicates that the subject has abnormal cell death and/or a disease relating to the cell. In some embodiments, the disease or condition is injury, inflammation, or cancer of the oligodendrocytes. In some embodiments, the disease is multiple sclerosis (MS).

In some embodiments, when the amount of cell-free DNA identified as being from a particular type of cell or cells, e.g., oligodendrocytes, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., oligodendrocytes is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as an oligodendrocyte, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

Group 12—Neuron CNS and oligodendrocytes

Also as provided in Table A, some genomic locations are uniformly under-methylated or over-methylated in a group of cells, namely neuron CNS and oligodendrocytes, as compared to all other cell types in the human.

In accordance with one embodiment of the present disclosure, a method is provided for identifying that a biological sample includes DNA from a cell selected from neuron CNS and oligodendrocytes. In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 5797-5870 or 5871-5898, or selected from SEQ ID NO: 5797-5870. In some embodiments, the method then identifies the target DNA fragment as being from a cell selected from neuron CNS and oligodendrocytes when no more than 40% of the CpG sites are methylated. In some embodiments, the method identifies the target DNA fragment as being from a cell selected from neuron CNS and oligodendrocytes when no more than 25%, 30%, 35%, 40%, 45%, or 50% of the CpG sites are methylated. Likewise, in some embodiments, the method identifies the target DNA fragment as being from a cell selected from neuron CNS and oligodendrocytes when at least 50%, 55%, 60%, 65%, 70%, 75%, 80%, 85% or 90% of the CpG sites are unmethylated. In some embodiments, the method identifies the target DNA fragment as not being from a cell selected from neuron CNS and oligodendrocytes when at least 50%, 55%, 60%, 65%, 70%, 75%, 80%, 85% or 90% of the CpG sites are methylated. In some embodiments, the method identifies the target DNA fragment as not being from a cell selected from neuron CNS and oligodendrocytes when no more than 25%, 30%, 35%, 40%, 45%, or 50% of the CpG sites are unmethylated.

In some embodiments, the method entails detecting the methylation status of a plurality (e.g., 3, 4, 5, 6, 7, 8, 9, 10 or more) of CpG sites of a target DNA fragment in the biological sample, wherein at least one (or at least two, three, four, five, six, seven, eight, nine, ten or all) of the CpG sites is located within, or within 100 bp, 200 bp, 500 bp or 1 kb from, a human genomic sequence selected from SEQ ID NO: 5899-5911, 5912-5912 or 5913-5923, or selected from SEQ ID NO: 5899-5911. In some embodiments, the method then identifies the target DNA fragment as being from a cell selected from neuron CNS and oligodendrocytes when 50% or more of the CpG sites are methylated. In some embodiments, the method identifies the target DNA fragment as being from a cell selected from neuron CNS and oligodendrocytes when at least 55%, 60%, 65%, 70%, 75%, 80%, 85% or 90% of the CpG sites are methylated. Likewise, in some embodiments, the method identifies the target DNA fragment as being from a cell selected from neuron CNS and oligodendrocytes when no more than 25%, 30%, 35%, 40%, 45%, or 50% of the CpG sites are unmethylated. In some embodiments, the method identifies the target DNA fragment as not being from a cell selected from neuron CNS and oligodendrocytes when no more than 25%, 30%, 35%, 40%, 45%, or 50% of the CpG sites are methylated. In some embodiments, the method identifies the target DNA fragment as not being from a cell selected from neuron CNS and oligodendrocytes when at least 50%, 55%, 60%, 65%, 70%, 75%, 80%, 85% or 90% of the CpG sites are unmethylated.

In some embodiments, the methylation status of one or more other DNA fragments is further used in the cell type determination. In some embodiments, the one or more additional (different from the first one) DNA fragment is represented by a genomic sequence of SEQ ID NO: 5797-5870, 5871-5898, 5899-5911, 5912-5912 or 5913-5923.

The cell type identification method can be used to detect disease or condition associated with the cell type. In one embodiment, when a cell-free DNA in a biological sample (e.g., blood, plasma, serum, semen, milk, urine, saliva or cerebral spinal fluid) is identified as being from a cell selected from neuron CNS and oligodendrocytes of a subject, the method indicates that the subject has abnormal cell death and/or a disease relating to the cell. In some embodiments, the disease or condition is injury, inflammation, or cancer of a cell selected from neuron CNS and oligodendrocytes.

In some embodiments, when the amount of cell-free DNA identified as being from a particular type of cell or cells, e.g., cells selected from neuron CNS and oligodendrocytes, is decreased, e.g., less at a second time point than at an earlier first time point of measurement, it indicates that the subject is recovering from the disease or condition. In some versions, the methods include making a diagnosis and/or treating a disease or condition accordingly based on the indication of recovery. In some embodiments, when the amount of cell-free DNA identified as being from a particular type of cell or cells, e.g., cells selected from neuron CNS and oligodendrocytes is increased, e.g., more at a second time point than at an earlier first time point of measurement, it indicates that the disease or condition is worsening. In some versions, the methods include making a diagnosis and/or treating a disease or condition accordingly based on the indication of worsening. In some embodiments, between two or more testing, the subject undergoes a treatment, and thus the testing result indicates the treatment effect.

Also provided, in one embodiment, is a method for determining the cell type of a disease cell, e.g., a cancer cell, the primary origin of the disease, e.g., cancer, cell, or the signal or origin of the disease, e.g., cancer, cell. In some embodiments, a cancer cell has unknown primary origin. In some embodiments, the methods include detecting the methylation status of one or more DNA fragment of the cancer cell and can use the methylation status to determine the cell as a cell selected from neuron CNS and oligodendrocytes, as described above.

In some instances, a cell-free DNA fragment is released from a cancer cell. The present technology can include determining the cell type of the cancer cell. In some embodiments, a genetic variation may also be present in the DNA fragment, and thus the cell type detection can help associate the genetic variation with the cancer. In some embodiments, the genetic variation includes a mutation. In some embodiments, the genetic variation includes a deletion or insertion. In some embodiments, the genetic variation constitutes microsatellite instability. In some embodiments, the genetic variation constitutes loss of heterozygosity. In some embodiments, the genetic variation interrupts or changes gene splicing. In some embodiments, the genetic variation causes frameshift or generation of premature stop codon. Once the primary origin of the cancer is identified, the subject may be treated with appropriate regiments for that cancer type.

Kits and Packages, Software Programs

The methods described herein may be performed, for example, by utilizing pre-packaged diagnostic kits, such as those described below, comprising agents which may be conveniently used to prepare DNA samples and detect DNA methylation.

DNA methylation detection can be performed with DNA isolated from cells or in situ directly upon tissue sections (fixed and/or frozen) of primary tissue such as biopsies obtained from biopsies or resections, such that no nucleic acid purification is necessary. The DNA molecules may also be cell-free DNA obtained from body fluid samples. Upon obtaining the DNA samples, in some embodiments, the DNA molecules may be fragmented or modified. In one embodiment, DNA modification agents are also provided, such as sodium bisulfite or APOBEC-Seq.

In one embodiment, a kit further includes instructions for use. In one aspect, a kit includes a manual comprising reference DNA methylation percentage cutoff levels.

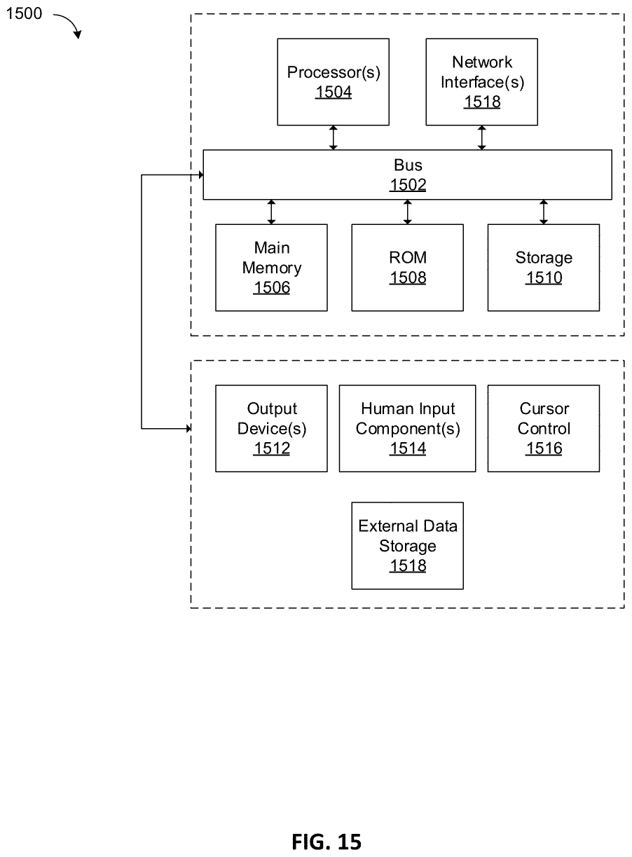

Also provided are computer programs for storing and/or analyzing the DNA methylation data. FIG. 15 is a block diagram that illustrates a computer system 1500 upon which any embodiments of the present and related technologies, such as DNA methylation data manipulation and analysis, may be implemented. The computer system 1500 includes a bus 1502 or other communication mechanism for communicating information, one or more hardware processors 1504 coupled with bus 1502 for processing information. Hardware processor(s) 1504 may be, for example, one or more general purpose microprocessors.

The computer system 1500 also includes a main memory 1506, such as a random access memory (RAM), cache and/or other dynamic storage devices, coupled to bus 1502 for storing information and instructions to be executed by processor 1504. Main memory 1506 also may be used for storing temporary variables or other intermediate information during execution of instructions to be executed by processor 1504. Such instructions, when stored in storage media accessible to processor 1504, render computer system 1500 into a special-purpose machine that is customized to perform the operations specified in the instructions.

The computer system 1500 further includes a read only memory (ROM) 1508 or other static storage device coupled to bus 1502 for storing static information and instructions for processor 1504. A storage device 1510, such as a magnetic disk, optical disk, or USB thumb drive (Flash drive), etc., is provided and coupled to bus 1502 for storing information and instructions.

The computer system 1500 may be coupled via bus 1502 to a display 1512, such as a LED or LCD display (or touch screen), for displaying information to a computer user. An input device 1514, including alphanumeric and other keys, is coupled to bus 1502 for communicating information and command selections to processor 1504. Another type of user input device is cursor control 1516, such as a mouse, a trackball, or cursor direction keys for communicating direction information and command selections to processor 1504 and for controlling cursor movement on display 1512. In some embodiments, the same direction information and command selections as cursor control may be implemented via receiving touches on a touch screen without a cursor. Additional data may be retrieved from the external data storage 1518.

The computer system 1500 may include a user interface module to implement a GUI that may be stored in a mass storage device as executable software codes that are executed by the computing device(s). This and other modules may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables.

In general, the word “module,” as used herein, refers to logic embodied in hardware or firmware, or to a collection of software instructions, possibly having entry and exit points, written in a programming language, such as, for example, Java, C or C++. A software module may be compiled and linked into an executable program, installed in a dynamic link library, or may be written in an interpreted programming language such as, for example, BASIC, Perl, or Python. It will be appreciated that software modules may be callable from other modules or from themselves, and/or may be invoked in response to detected events or interrupts. Software modules configured for execution on computing devices may be provided on a computer readable medium, such as a compact disc, digital video disc, flash drive, magnetic disc, or any other tangible medium, or as a digital download (and maybe originally stored in a compressed or installable format that requires installation, decompression or decryption prior to execution). Such software code may be stored, partially or fully, on a memory device of the executing computing device, for execution by the computing device. Software instructions may be embedded in firmware, such as an EPROM. It will be further appreciated that hardware modules may be comprised of connected logic units, such as gates and flip-flops, and/or may be comprised of programmable units, such as programmable gate arrays or processors. The modules or computing device functionality described herein can be implemented as software modules, but may be represented in hardware or firmware. Generally, the modules described herein refer to logical modules that may be combined with other modules or divided into sub-modules despite their physical organization or storage.

The computer system 1500 may implement the techniques described herein using customized hard-wired logic, one or more ASICs or FPGAs, firmware and/or program logic which in combination with the computer system causes or programs computer system 1500 to be a special-purpose machine. According to one embodiment, the techniques herein are performed by computer system 1500 in response to processor(s) 1504 executing one or more sequences of one or more instructions contained in main memory 1506. Such instructions may be read into main memory 1506 from another storage medium, such as storage device 1510. Execution of the sequences of instructions contained in main memory 1506 causes processor(s) 1504 to perform the process steps described herein. In alternative embodiments, hard-wired circuitry may be used in place of or in combination with software instructions.

The term “non-transitory media,” and similar terms, as used herein refers to any media that store data and/or instructions that cause a machine to operate in a specific fashion. Such non-transitory media may comprise non-volatile media and/or volatile media. Non-volatile media includes, for example, optical or magnetic disks, such as storage device 1510. Volatile media includes dynamic memory, such as main memory 1506. Common forms of non-transitory media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, and networked versions of the same.

Non-transitory media is distinct from but may be used in conjunction with transmission media. Transmission media participates in transferring information between non-transitory media. For example, transmission media includes coaxial cables, copper wire and fiber optics, including the wires that comprise bus 1502. Transmission media can also take the form of acoustic or light waves, such as those generated during radio-wave and infra-red data communications.

Various forms of media may be involved in carrying one or more sequences of one or more instructions to processor 1504 for execution. For example, the instructions may initially be carried on a magnetic disk or solid-state drive of a remote computer. The remote computer can load the instructions into its dynamic memory and send the instructions over a telephone line using a component control. A component control local to computer system 1500 can receive the data on the telephone line and use an infra-red transmitter to convert the data to an infra-red signal. An infra-red detector can receive the data carried in the infra-red signal and appropriate circuitry can place the data on bus 1502. Bus 1502 carries the data to main memory 1506, from which processor 1504 retrieves and executes the instructions. The instructions received by main memory 1506 may retrieve and execute the instructions. The instructions received by main memory 1506 may optionally be stored on storage device 1510 either before or after execution by processor 1504.

The computer system 1500 also includes a communication interface 1518 coupled to bus 1502. Communication interface 1518 provides a two-way data communication coupling to one or more network links that are connected to one or more local networks. For example, communication interface 1518 may be an integrated services digital network (ISDN) card, cable component control, satellite component control, or a component control to provide a data communication connection to a corresponding type of telephone line. As another example, communication interface 1518 may be a local area network (LAN) card to provide a data communication connection to a compatible LAN (or WAN component to communicated with a WAN). Wireless links may also be implemented. In any such implementation, communication interface 1518 sends and receives electrical, electromagnetic or optical signals that carry digital data streams representing various types of information.

A network link typically provides data communication through one or more networks to other data devices. For example, a network link may provide a connection through local network to a host computer or to data equipment operated by an Internet Service Provider (ISP). The ISP in turn provides data communication services through the world-wide packet data communication network now commonly referred to as the “Internet”. Local network and Internet both use electrical, electromagnetic or optical signals that carry digital data streams. The signals through the various networks and the signals on network link and through communication interface 1518, which carry the digital data to and from computer system 1500, are example forms of transmission media.

The computer system 1500 can send messages and receive data, including program code, through the network(s), network link and communication interface 1518. In the Internet example, a server might transmit a requested code for an application program through the Internet, the ISP, the local network and the communication interface 1518.

The received code may be executed by processor 1504 as it is received, and/or stored in storage device 1510, or other non-volatile storage for later execution. Each of the processes, methods, and algorithms described in the preceding sections may be embodied in, and fully or partially automated by, code modules executed by one or more computer systems or computer processors comprising computer hardware. The processes and algorithms may be implemented partially or wholly in application-specific circuitry.

The various features and processes described above may be used independently of one another, or may be combined in various ways. All possible combinations and sub-combinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

Any process descriptions, elements, or blocks in the flow diagrams described herein and/or depicted in the attached figures should be understood as potentially representing modules, segments, or portions of code which include one or more executable instructions for implementing specific logical functions or steps in the process. Alternate implementations are included within the scope of the embodiments described herein in which elements or functions may be deleted, executed out of order from that shown or discussed, including substantially concurrently or in reverse order, depending on the functionality involved, as would be understood by those skilled in the art.

It should be emphasized that many variations and modifications may be made to the above-described embodiments, the elements of which are to be understood as being among other acceptable examples. All such modifications and variations are intended to be included herein within the scope of this disclosure. The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the embodiments should, therefore, be construed in accordance with the appended claims and any equivalents thereof.

The various operations of example methods described herein may be performed, at least partially, by one or more processors that are temporarily configured (e.g., by software) or permanently configured to perform the relevant operations. Similarly, the methods described herein may be at least partially processor-implemented, with a particular processor or processors being an example of hardware. For example, at least some of the operations of a method may be performed by one or more processors. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an Application Program Interface (API)).

Treatments

In another embodiment, the methylation status can be used to make or influence a clinical decision (e.g., diagnosis of cancer, treatment selection, assessment of treatment effectiveness, etc.). For example, a physician can prescribe an appropriate treatment (e.g., a resection surgery, radiation therapy, chemotherapy, and/or immunotherapy).

In some embodiments, the treatment is one or more cancer therapeutic agents selected from the group consisting of a chemotherapy agent, a targeted cancer therapy agent, a differentiating therapy agent, a hormone therapy agent, and an immunotherapy agent. For example, the treatment can be one or more chemotherapy agents selected from the group consisting of alkylating agents, antimetabolites, anthracyclines, anti-tumor antibiotics, cytoskeletal disruptors (taxans), topoisom erase inhibitors, mitotic inhibitors, corticosteroids, kinase inhibitors, nucleotide analogs, platinum-based agents and any combination thereof. In some embodiments, the treatment is one or more targeted cancer therapy agents selected from the group consisting of signal transduction inhibitors (e.g. tyrosine kinase and growth factor receptor inhibitors), histone deacetylase (HD AC) inhibitors, retinoic receptor agonists, proteosome inhibitors, angiogenesis inhibitors, and monoclonal antibody conjugates. In some embodiments, the treatment is one or more differentiating therapy agents including retinoids, such as tretinoin, alitretinoin and bexarotene. In some embodiments, the treatment is one or more hormone therapy agents selected from the group consisting of anti-estrogens, aromatase inhibitors, progestins, estrogens, anti-androgens, and GnRH agonists or analogs. In one embodiment, the treatment is one or more immunotherapy agents selected from the group comprising monoclonal antibody therapies such as rituximab (RITUXAN) and alemtuzumab (CAMPATH), non-specific immunotherapies and adjuvants, such as BCG, interleukin-2 (IL-2), and interferon-alfa, immunomodulating drugs, for instance, thalidomide and lenalidomide (REVLIMID). It is within the capabilities of a skilled physician or oncologist to select an appropriate cancer therapeutic agent based on characteristics such as the type of tumor, cancer stage, previous exposure to cancer treatment or therapeutic agent, and other characteristics of the cancer.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1: A Human DNA Methylation Atlas Reveals Design Principles of Cell Type-Specific Methylation and Identifies Thousands of Cell Type-Specific Regulatory Elements

This example describes the generation of a human methylome atlas, based on deep whole-genome bisulfite sequencing of 39 cell types sorted from 207 healthy tissue samples.

Replicates of the same cell type are >99.5% identical, demonstrating robustness of cell identity programs to genetic variation and environmental perturbation. Unsupervised clustering of the atlas recapitulates key elements of tissue ontogeny, and identifies methylation patterns retained since gastrulation. Loci uniquely unmethylated in an individual cell type often reside in transcriptional enhancers and contain DNA binding sites for tissue-specific transcriptional regulators. Uniquely methylated loci are rare and are enriched for CpG islands, polycomb targets, and CTCF binding sites, suggesting a role in shaping cell type-specific chromatin looping. The atlas provides an essential resource for interpretation of disease-associated genetic variants, and a wealth of potential tissue-specific biomarkers for use in liquid biopsies.

### Methods and Materials

Human Tissue Samples

Human tissues were obtained from various sources. The majority (150) of the 207 samples analyzed were sorted from tissue remnants obtained at the time of routine, clinically indicated surgical procedures at the Hadassah Medical Center. In all cases, normal tissue distant from any known pathology was used. Surgeons and/or pathologists were consulted prior to removing the tissue to confirm that its removal would not compromise the final pathologic diagnosis in any way. For example, in patients undergoing right colectomy for carcinoma in the cecum, the distal most part of the ascending colon and the most proximal part of the terminal ileum were obtained for cell isolation. Normal bone marrow was obtained at the time of joint replacement in patients with no known hematologic pathology. The patient population included 137 individuals (n=61 males, n=75 females), aged 3-83 years. The majority of donors were Caucasian. Approval for collection of normal tissue remnants was provided by the Institutional Review Board (IRB, Helsinki Committee), Hadassah Medical Center, Jerusalem, Israel. Written informed consent was obtained from each donor or legal guardian prior to surgery.

### Tissue Dissociation and FACS Sorting of Purified Cell Populations

Fresh tissue obtained at the time of surgery was trimmed to remove extraneous tissue. Cells were dispersed using enzyme-based protocols optimized for each tissue type. The resulting single-cell suspension was incubated with the relevant antibodies and FACS sorted to obtain the desired cell type (Table 1).

TABLE 1
Listing of Cell Types

| Group | Group Name | Cell type |
|---|---|---|
| A | Gastro-Intestinal system | Oral, Larynx and Esophageal epithelium |
| A | Gastro-Intestinal system | Gastric Epithelium |
| A | Gastro-Intestinal system | Small Intestine Epithelium |
| A | Gastro-Intestinal system | Colon Epithelium |
| A | Gastro-Intestinal system | Colon Fibroblasts |
| A | Gastro-Intestinal system | Gallbladder Epithelium |
| A | Gastro-Intestinal system | Liver Hepatocytes |
| A | Gastro-Intestinal system | Pancreatic Acinar cells |
| A | Gastro-Intestinal system | Pancreatic Alpina cells |
| A | Gastro-Intestinal system | Pancreatic Beta cells |
| A | Gastro-Intestinal system | Pancreatic Delta cells |
| A | Gastro-Intestinal system | Pancreatic Ductal cells |
| B | Genito-urinary | Endometrium Epithelium |
| B | Genito-urinary | Fallopian Epithelium |
| B | Genito-urinary | Kidney Epithelium |
| B | Genito-urinary | Bladder Epithelium |
| B | Genito-urinary | Prostate Epithelium |
| B | Genito-urinary | Breast Basal Epithelium |
| B | Genito-urinary | Breast Luminal Epithelium |
| C | Cardio-Vascular-Pulmonary | Lung Alveolar Epithelium |
| C | Cardio-Vascular-Pulmonary | Lung Bronchial Epithelium |
| C | Cardio-Vascular-Pulmonary | Heart Cardiomyocytes |
| C | Cardio-Vascular-Pulmonary | Heart Fibroblasts |
| C | Cardio-Vascular-Pulmonary | Vascular Endothelial cells |
| D | Hemo | Blood B cells |
| D | Hemo | Blood Gramlocytes |
| D | Hemo | Blood Monocytes + Macrophages |
| D | Hemo | Blood NK cells |
| D | Hemo | Blood T cells |
| D | Hemo | Erythrocyte progenitor cells |
| E | Dermal-Skeleto-muscular | Epidermal Keratinocytes |
| E | Dermal-Skeleto-muscular | Dermal Fibroblasts |
| E | Dermal-Skeleto-muscular | Osteoblasts |
| E | Dermal-Skeleto-muscular | Skeletal Muscle cells |
| E | Dermal-Skeleto-muscular | Smooth Muscle cells |
| F | Neural-Misc. | Thyroid Epithelium |
| P | Neural-Misc. | Adipocytes |
| F | Neural-Misc. | Neuron CNS |
| F | Neural-Misc. | Oligodendrocytes |

TABLE 2
Composite Cell Types

| Group | Composite Types |
|---|---|
| 1 | Neurons + Oligodendrocytes |
| 2 | Pancreatic Alpha + Beta + Della cells |
| 3 | Breast Basal + Luminal Epithelium |
| 4 | Lung Alveolar + Bronchial cells |
| 5 | Fallopian + Ovary Epithelium |
| 6 | Gastric + Small Intes. + Colon Epithelium |
| 7 | Gastric + Small Intes, Epithelium |
| 8 | Small Intes. + Colon Epithelium |
| 9 | Colon + Heart Fibroblasts |
| 10 | Cardiomyocytes + Skeletal + Smooth muscle cells |
| 11 | Skeletal + Smooth muscle cells |
| 12 | Heart Cardiomvocytes + Fibroblasts |

Purity of live sorted cells was determined by mRNA analysis for key known cell-type specific genes whereas purity of cells that were fixed prior to sorting was determined using previously validated cell-type specific methylation signals. DNA was extracted using the DNeasy Blood and Tissue kit (#69504 Qiagen; Germantown, Md.) according to the manufacturer's instructions, and stored at −20° C. for bisulfite conversion and whole genome sequencing.

### Whole-Genome Bisulfite Sequencing

Up to 75 ng of sheared gDNA was subjected to bisulfite conversion using the EZ-96 DNA Methylation Kit (Zymo Research; Irvine, Calif.), with liquid handling on a Hamilton MicroLab STAR (Hamilton; Reno, Nev.). Dual indexed sequencing libraries were prepared using Accel-NGS Methyl-Seq DNA library preparation kits (Swift BioSciences; Ann Arbor, Mich.) and custom liquid handling scripts executed on the Hamilton MicroLab STAR. Libraries were quantified using KAPA Library Quantification Kits for Illumina Platforms (Kapa Biosystems; Wilmington, Mass.). Four uniquely dual indexed libraries, along with 10% PhiX v3 library (Illumina; San Diego, Calif.), were pooled and clustered on an Illumina NovaSeq 6000 S2 flow cell followed by 150-bp paired-end sequencing.

### Whole-Genome Bisulfite Sequencing Computational Processing

Paired-end FASTQ files were mapped to the human (hg19), lambda, pUC19 and viral genomes using bwa-meth (V 0.2.0), with default parameters, then converted to BAM files using SAMtools (V 1.9). Duplicated reads were marked by Sambamba (V 0.6.5), with parameters “-1 l -t 16-sort-buffer-size 16000-overflow-list-size 10000000”. Reads with low mapping quality, duplicated, or not mapped in a proper pair were excluded using SAMtools view with parameters -F 1796-q 10. Reads were stripped from non-CpG nucleotides and converted to PAT files using wghstools (V 0.1.0).

Genomic Segmentation into Multi-Sample Homogenous Blocks

We developed and implemented a multi-channel Dynamic Programming segmentation algorithm to divide the genome into continuous genomic regions (blocks) showing homogeneous methylation levels across multiple CpGs, for each sample. We modeled the CpG sites with a generative probabilistic model, assuming there is a universal underlying segmentation of all ˜28M sites into an unknown number of blocks. This segmentation, unlike the methylation patterns, is similar across different cell types and individuals. Each block induces a Bernoulli distribution with some θᵢ^{k}, where i is a block index and k is a sample (k=1, . . . , K), and all CpG sites are represented by random variables sampled i.i.d from the same beta value Ber(θᵢ^{k}).

### Finding an Optimal Segmentation

We used dynamic programming to find a segmentation that maximizes a log-likelihood score for the blocks. The score for the i'th block is the log-likelihood of the beta values of the sites in this block across all K samples. We computed K Bayesian estimators for the block's parameters \hat{θ}ᵢ^{k}:

$${\hat{θ}}_{i}^{k} = \frac{{( {N}_{C} )}_{i}^{k} + {α}_{C}}{{( {N}_{C} )}_{i}^{k} + {( {N}_{T} )}_{i}^{k} + {α}_{C} + {α}_{T}}$$

where (N_{C})ᵢ^{k}, (N_{T})ᵢ^{k} is the number of observations of sites in the block i and sample k that are methylated/unmethylated. α_{C}, α_{T} are pseudocounts for methylated/unmethylated observations in block i. They are constant hyper-parameters of the model, which set the tradeoff between longer to more homogenous blocks. The log-likelihood of a single block in a single example is:

score(blockᵢ)=llᵢ=Σ^{K}ₖ₌₁((N_{C})ᵢ^{k}·log(\hat{θ}ᵢ^{k})+(N_{T})ᵢ^{k}·log(1−θᵢ^{k})).

### Dynamic Programming Algorithm

We maintained a 1×N table T for the optimal scores (N=28,217,448). T[i] holds the score of the optimal segmentation of sites 1, . . . , i. T[N] holds the final optimal score. The table is updated from 1 to N as follows:

$$T [ i ] = {max}_{i ′ < i} \{ T [ {i}^{′} ] + score ( block [ {i}^{′} + 1 , … , i ] ) \}$$

T[i] is the maximum over the sites preceding site i (i′<i), of the score of the optimal segmentation that ends on site i′(T[i′]), concatenated with the single block from i′+1 to i. A similar traceback table is also maintained, in order to retrieve the optimal segmentation. In order to improve performance, we set an upper bound on block length (either in CpG sites or bases), which improves running time and allows for multi-processing.

### Segmentation and Clustering Analysis

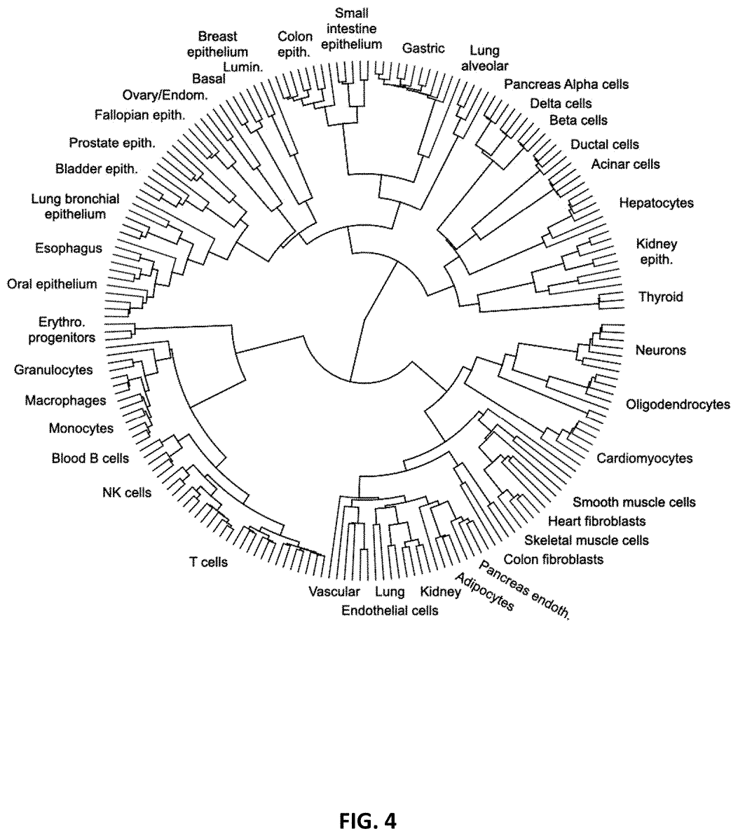

We segmented the genome into 7,264,350 blocks using wgbstools (with parameters “segment-max_bp 5000”), with all of the 207 samples as reference, and retained 2,107,635 blocks that cover ≥4 CpGs. For the hierarchical clustering we selected the top 1% (21,077) blocks showing the largest variability in average methylation across all samples. Blocks with sufficient coverage of ≥10 observations (calculated as sequenced CpG sites) across 3 of the samples we further retained. We then computed the average methylation for each block and sample calculated using wgbstools (“-beta_to_table-c 10”), marked blocks with <10 observations as missing values, and imputed their methylation values using sklearn KNNImputer (V 0.24.2). The 207 samples were clustered with the UPGMA clustering algorithm, using scipy (V 1.6.3), and Li norm as the distance metric. The fanning diagram (FIG. 4) was plotted using ggtree (V 2.2.4).

### Cell Type-Specific Markers

The 207 atlas samples were grouped into 51 groups by their cell type, including 39 basic groups (e.g. epithelial cells Pancreatic Alpha cells, Table 1), and composite super-groups (e.g. epithelial Alpha, Beta, and Delta cells, all from the endocrine pancreas, Table 2). We performed a one-vs-all comparison, to identify differentially methylated blocks unique for each set. For this, we first identified blocks that cover ≥5 CpGs, with length varying between 10 to 500 bp. We then calculated the average methylation per block/sample, as the number of methylated CpGs sites within all sequenced reads across each block). Blocks with insufficient coverage (<25 observations) were assigned a default value of 0.5. We then selected blocks with average methylation below 0.33 across samples from one cell type, with an average methylation of ≥0.66 in all others, or vice versa.

For cell type-specific markers, we selected the top 25, 100 or 250 blocks with the highest delta beta for each cell type. For hypo-methylated markers this was computed as the difference between the 75^{th} percentile among the block average methylation within samples in the target set, and the 2.5^{th} percentile among the rest of samples (background set). This allowed for ˜1 outlier sample in the target group, and ˜5 outliers outside. Analogously, for hyper-methylated markers we computed the 97.5^{th} percentile of the background and the 25^{th} percentile within the target samples.

### Enrichment for Gene Set Annotations

Analysis of gene set enrichment was performed using GREAT (McLean et al., (2010) Nat. Biotechnol. 28, 495-501). For each cell type, we selected the top 250 differentially unmethylated regions, and ran GREAT via batch web interface using default parameters. Enrichments for “Ensembl Genes” were ignored, and a significance threshold of Binomial FDR≤0.05 was used.

### Enrichment for Chromatin Marks

For each cell type, we analyzed the top 250 differentially unmethylated regions vs. published ChIP-seq (H3K27ac and H3K4me1) and DNase-seq from the Roadmap Epigenomics project. These include E032 for B cells markers, E034 for T cells markers, E029 for monocytes/macrophages markers, E066 for liver hepatocytes, E104 for heart cardiomyocytes and fibroblasts, E109 and E110 for gastric/small intestine/colon. Raw single-cell ATAC-seq data were downloaded from GEO GSE165659 as “feature” and “matrix” files for 70 samples. For each sample, cells of the same type were pooled together to output a bedGraph file, which was mapped from hg38 to hg19 using UCSC liftOver. Overlapping regions were dropped using bedtools (V 2.26.0). Finally, bigWig files were created using bedGraphToBigWig (V 4). Heatmaps and average plots were prepared using deepTools (V 3.4.1), with the computeMatrix, plotHeatmap, and plotProfile functions. We used default parameters except for -referencePoint=center, 15 Kb margins, and binSize=200 for ChIP-seq, DNaseI and chromHMM data, and 75 Kb margins with binSize=1000 for ATAC-seq data.

### Motif Analysis

For each cell type, we analyzed the top 250 differentially unmethylated regions for known motifs, using HOMER's findMotifsGenome.pl function, with -bits and -size 250 parameters. Additionally, we analyzed the top 100 differentially methylated regions for each cell type (using the same parameters), as well as their combined set, composed of 3,125 regions in total.

### Methylation Marker-Gene Associations

For each cell type-specific marker, we identified all neighboring genes, up to 500 Kb apart. We then examined the expression levels of these genes across the GTEx datasets, covering 50 tissues and cell types. We then calculated the over-expression level of each <gene,condition> pair, by computing the deviation (Z-score) of that gene across all conditions (row-wise calculation), and then the deviation of that condition across all genes (column-wise calculation), repeatedly until convergence. This Z-score reflects the bidirectional enrichment of each <gene,condition> combination, compared to all other genes/conditions. We then classified each <marker,gene,condition> combination as Tier 1: distance≤5 Kb, expression≥10 TPM, and Z-score≥1.5; or Tier 2: same but as Tier 1, with dist≤50 Kb; or Tier 3: up to 750 Kb, expression≥25 TPM, and Z-score≥5: or Tier 4: same as Tier 3 with Z-score≥3.5

### Inter-Individual Variation in Cell Type Methylation

We defined a similarity score between two samples as the fraction of blocks containing ≥3 CpGs, and ≥10 binary observations (sequenced CpG sites), where the average methylation of the two samples differs by ≥0.5. Only cell types with n≥3 FACS-sorted replicates from different donors are considered (138 samples in total).

### CTCF ChIP-Seq Analysis

CTCF ChIP-seq data were downloaded from the ENCODE project, as 168 bigwig files, covering 61 tissues/cell types (hg19). Samples of the same cell type were averaged using multiBigwigSummary bins (V 3.4.1).

### Endodermal Marker Analysis

The 776 endodermal hypo-methylated markers were found using wgbstools' find_markers function (V 0.1.0), with parameters “-delta 0.4-tg_quant 0.1-bg_quant 0.1. Endoderm-derived epithelium (51 samples was compared to 105 non-epithelial samples from mesoderm or ectoderm. Blocks were selected as markers if the average methylation of the 90^{th} percentile of the epithelial samples was lower than the 10^{th} percentile of the non-epithelial samples by at least 0.4.

### Results

### A Comprehensive Methylation Atlas of Primary Human Cell Types

To portray the genome-wide patterns of DNA methylation across a variety of cell types, we obtained 207 samples of freshly isolated healthy adult tissue samples from 137 consented donors undergoing a variety of surgical procedures (ages 3-83). We dissociated tissue samples into single cell suspensions and used lineage-specific antibodies to cell type-specific surface markers to FACS purify cell populations covering 39 primary cell types. The purity of cell types was confirmed using RT-qPCR for cell type specific gene expression markers and known tissue-specific methylation markers were assessed when possible. We then subjected cell type-specific genomic DNA to WGBS and sequenced at a mean depth of >30×, using 150 bp-long paired-end reads, with an average fragment size of 174 bp.

Sequencing reads were mapped to the human genome (hg19). Duplicated reads, reads not covering any CpG site, and reads not mapped in a proper pair with a high mapping quality were filtered out.

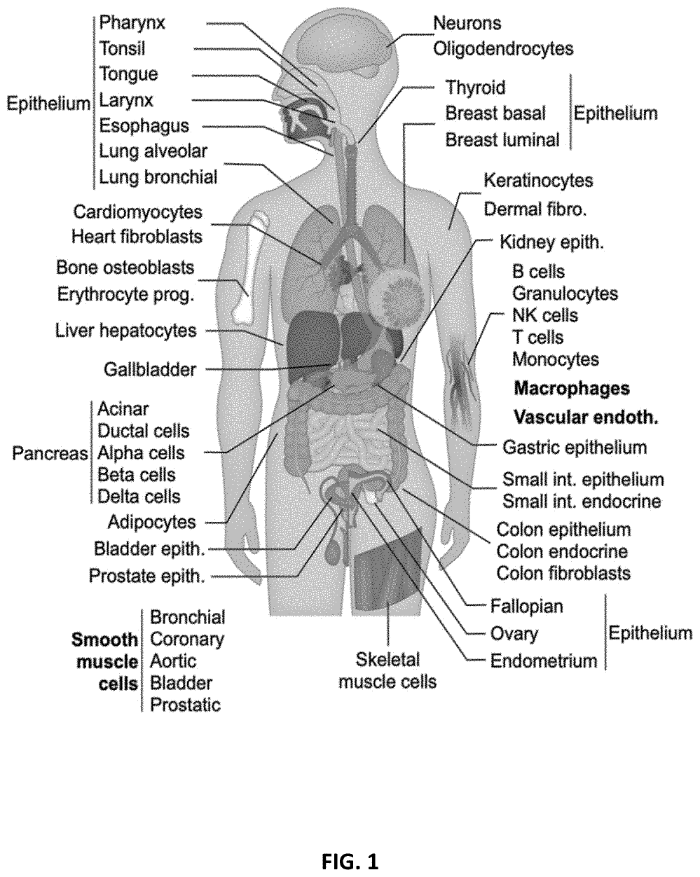

Overall we characterized the methylomes of 39 types of cells (Table 1). These include various blood cell types (T cells, B cells, NK cells, granulocytes, monocytes, and tissue-resident macrophages); erythrocyte progenitor cells; hepatocytes; exocrine and endocrine pancreatic cell types; epithelial cells from the lung (alveolar and bronchial), breast (basal and luminal), kidney, mouth, esophagus, thyroid, bladder, and prostate; neurons and oligodendrocytes; adipocytes; gastrointestinal epithelium from different segments of the GI tract; endometrial, fallopian and ovarian epithelium; cardiomyocytes, skeletal, and various anatomical sources of smooth muscle and vascular endothelial cells (FIG. 1). These represent nearly all major human cell types, allowing a composite view of physiological systems (e.g., GI tract, hematopoietic cells, pancreas), as well as a comparison of similar cell types in different environments (e.g., tissue-resident macrophages).

### Identification of Human Methylation Blocks

It was observed that the 207 methylomes showed great similarities between replicates, with distinctive changes between cell types in a block-like manner. We therefore sought to identify and delineate genomic regions that are differentially methylated in specific cell types. These would shed light on biological processes that are unique to specific cell types, define their cellular identity, and could be further used as tissue-specific methylation biomarkers to identify the cellular origin of circulating cell-free DNA fragments.

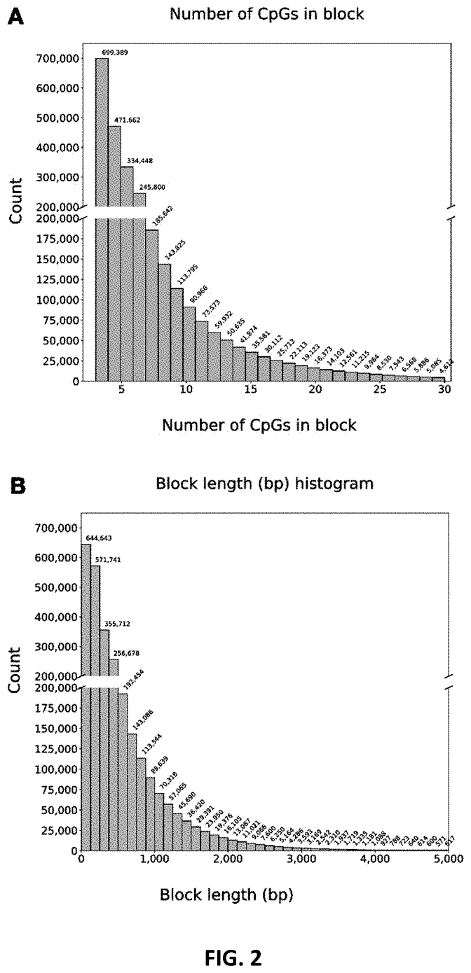

We developed wgbstools, a computational machine learning suite to represent, compress, visualize, and analyze WGBS data. Our first goal was to move to a more compact representation of the genome-wide methylomes. Instead of using fixed-width genomic windows as is typically done in differentially methylated region (DMR) calling, we sought an unbiased approach that would automatically identify natural changepoints in DNA methylation patterns across multiple conditions. For this, we developed a computational multi-channel dynamic programming algorithm to optimally segment the genome into 7,264,350 non-overlapping continuous blocks. Each of these blocks spans highly correlated CpG sites that share similar methylation patterns in each of the 207 samples analyzed, but may co-vary across cell types. We then filtered out all single and double-CpG blocks to retain 2,807,024 methylation blocks, with an average block length of 532 bp (IQR=551 bp) spanning 8 CpGs on average (IQR=5 CpGs, FIG. 2). These blocks represent compact units that are more straightforward to robustly analyze than individual CpG sites. Beyond the technical ease, the regional nature of DNA methylation strongly suggests that these methylation blocks are the biological “atoms” of human DNA methylation.

### The Extent of Inter-Individual Variation in Cell Type Methylation

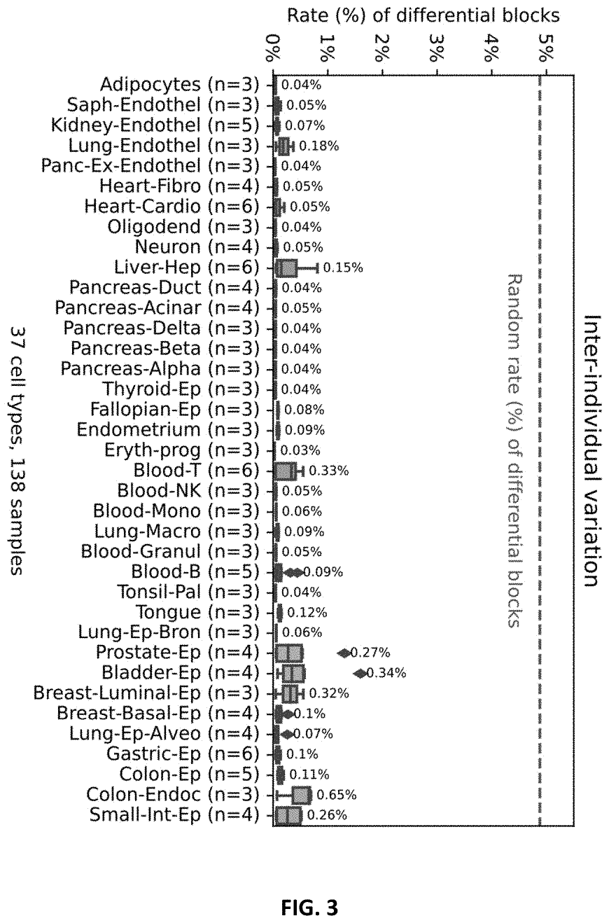

We asked how robust the methylation patterns of a given cell type are across different individuals. This serves a technical goal of defining reproducibility of preparations, but is also addressing a fundamental biological question: how much of the epigenome is determined by cell type-specific differentiation programs as opposed to genetic or environmental factors? For this, we focused on all methylation blocks that consisted of 3 CpGs or more, and calculated for each pair of samples how many blocks show an absolute difference of 50% or more in their average methylation. For most cell types, less than 0.5% of the blocks differ in methylation between donors, compared to an average variation of 4.9%/o blocks among samples of different cell types (FIG. 3). This suggests high similarity in DNA methylation across donors, on par with the estimated variability of the genome sequence between individuals. Importantly, the same inter-individual variation was observed in replicates obtained from different laboratories. While this definition of variation (as 50%) is somewhat arbitrary, other thresholds (35%-50%) show a similar trend, with ≤0.5% of variable blocks.

Two hundred and one samples in the methylation atlas had n≥3 biological replicates of the same cell type. Strikingly, for 200 of these (99.5%), the most similar sample is of the same cell type from another individual. These results demonstrate the purity and reproducibility of cell preparations used in developing the methylation atlas, and indicate high inter-individual similarity of normal cell type methylomes.

### Methylation Patterns Record Human Developmental History

DNA methylation patterns are shaped and largely fixed during cell differentiation, and hence reflect the epigenetic identity of a cell. However, methylation patterns could also reflect the developmental history of cells. For example, the differentiated progeny of a progenitor cell may retain methylation marks that were used to control genes expressed in that progenitor, even though these genes are no longer active in the differentiated cells. The implication would be that DNA methylation can be used as an endogenous lineage tracer, similarly to somatic mutation profiles. We thus used the large collection of cell type-specific methylomes to test the hypothesis that the methylome of a given cell type reflects its lineage history.

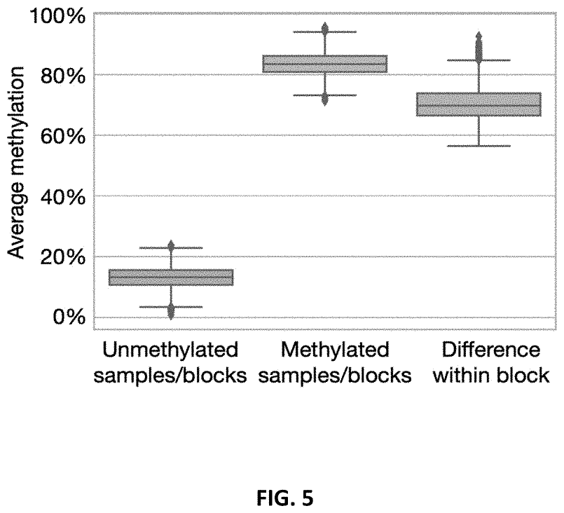

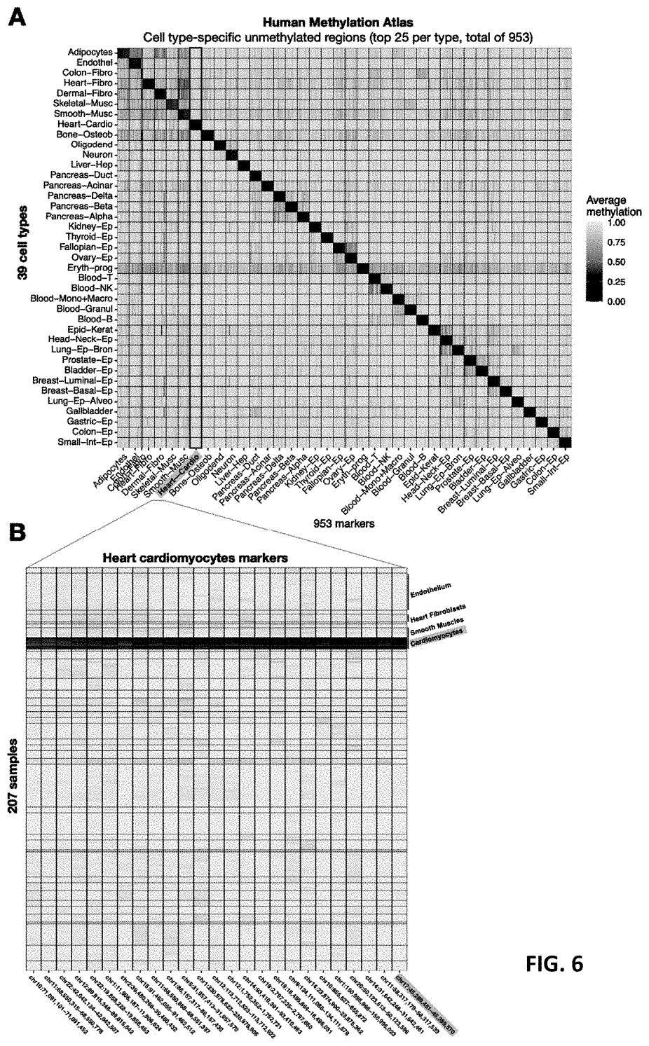

We focused on blocks containing 4 CpGs or more, calculated the average methylation levels per sample, and selected those showing the highest variability (21K blocks, top 1%) across all samples (FIG. 5). We then clustered all 207 methylomes using an unsupervised agglomerative algorithm (UPGMA) that iteratively identifies and connects the two closest samples, regardless of their labeling. This blinded clustering analysis systematically grouped together biological replicates of the same cell type. This further supports reproducibility of tissue preparation and cell sorting, and suggests that 3-4 replicates of each normal cell type are sufficient to infer its genome-wide methylation patterns for practical purposes such as marker identification. Notably, clustering based on other sets of high-variability blocks (top 1.5% through top 10%) produced similar groupings.

Strikingly, the resulting fanning diagram (FIG. 4) recapitulated key elements of lineage relationships among human tissues. For example, different pancreatic islet cell types (alpha, beta, delta), which are known to be derived from the same embryonic endocrine progenitor cell type, densely cluster together. Islet cells share endodermal developmental origins, but not function, with the exocrine pancreas (acinar cells and ducts) and the liver. Consistent with methylomes reflecting lineage rather than function, islet cells are clustered with pancreatic duct and acinar cells, and then with hepatocytes. Importantly, the phenotype of islet cells has many common features with neurons, including both tissue-specific transcription factors and functional elements such as exocytosis controlled by voltage-dependent calcium signaling. However, neurons and islet cells derive from different germ layers (ectoderm and endoderm, respectively). The methylomes of islet cells and neurons have little in common, indicating that methylation mostly reflects lineage rather than function. Additional examples for lineages reconstructed by methylation include the clustering of gastric, small intestine and colon epithelial cells; the clustering of all blood cell types; and the clustering of multiple mesoderm-derived cell types including vascular endothelial cells, adipocytes and skeletal muscle. The map also reveals intriguing relationships between cell types that are not known to share neither function nor lineage, such as the clustering of brain cell types (neurons and oligodendrocytes) with cardiomyocytes. Interestingly, lung bronchial epithelium clustered along with esophagus and oral epithelium consistent with shared embryonic origin whereas alveolar epithelium clustered with intestinal epithelium suggesting a common embryologic origin distinct from that of bronchial epithelium. This is consistent with recent lineage tracing experiments which showed early lineage specification of alveolar cell lineage, although a common lineage with gastric epithelium was not addressed.

Some methylation patterns were common to multiple cell types which have separated during very early stages of development. For example, 776 blocks are remarkably unmethylated in epithelial cell types derived from early endodermal derivatives, and methylated in cell types derived from the mesoderm and the ectoderm. The most likely interpretation of this observation is that these sites were demethylated in the endoderm germ layer of all donors, during gastrulation or shortly thereafter. Many decades later, different endoderm-derived cell types in different individuals still retain these embryonic patterns. Since endoderm derivatives do not share common function or gene expression, this provides yet another striking example of methylation patterns as a stable lineage mark. Methylation patterns reflected also later lineage splits. For example, lymphocytes (T, B and NK cells) clustered together, separately from myeloid cells (macrophage, monocyte and granulocytes).

Finally, we applied the same segmentation and blinded clustering approach to a published methylation atlas from the Roadmap Epigenomics project (Kundaje, A. et al. (2015) Nature 518, 317-330). The algorithm failed to group together related tissues and cell types, often clustering samples based on donor identity rather than type. This further emphasizes the importance of careful cell sorting and purification into homogeneous cell types, avoiding whole-tissue and mixed cell populations.

### Tens to Hundreds of Methylation Blocks Uniquely Characterize Each Cell Type

We next turned to study the methylomes of individual cell types, and identify genomic regions that are differentially methylated in a cell type-specific manner. Based on the unsupervised clustering, we organized the 207 samples into 39 groups of specific cell types, including blood cell types (B, T, NK, Granulocytes, monocytes and tissue-resident macrophages), breast epithelial cells (basal or luminal), lung epithelium (alveolar or bronchial), pancreatic endocrine (alpha, beta, delta) or exocrine (acinar and duct) cells, vascular endothelial cells from various sources, cardiomyocytes and cardiac fibroblasts, and more. We also defined 12 super-groups, where related cell types were grouped together, including muscle cells, gastrointestinal epithelium, pancreatic cells, and more (Tables 1-2).

We then focused on differentially methylated blocks, composed of 5 CpGs or more, that are methylated (average methylation in block ≥66%) in one group of cell types, but unmethylated (≤33%) in all other samples, or vice versa. Overall, we identified 11,125 differentially methylated genomic regions. Intriguingly, almost all regions (98%, 10,892) were unmethylated in one cell type, and methylated in all others. While some cell types show a surprisingly high number of differential regions, including hepatocytes (1,111 uniquely methylated or unmethylated regions), cardiomyocytes (1,084 regions), oligodendrocytes (897 regions), and small intestinal/colon epithelial cells (811 regions), other cell types had much fewer uniquely methylated regions. For example, there were only 91 unique regions in T cells, 51 in NK cells, 84 in pancreatic alpha cells, 61 in pancreatic duct cells, and 34 in pancreatic delta cells. Only three uniquely methylated regions (at these thresholds) were found for skeletal muscle cells, and no joint markers were found for smooth muscle cells, endothelial cells, or fibroblasts. Obviously, these results are affected by the overall composition of the DNA methylation atlas, allowing more unique regions for cell types with no immediate neighbors. Nonetheless, the findings may reflect the extent to which a particular cell type is unique in its differentiated function relative to other cell types. For example, cardiomyocytes apparently have a large number of specialized functions, reflected in their epigenetic makeup, while pancreatic alpha cells may have much fewer unique functions (given that the atlas contains the highly similar beta and delta cells). Interestingly, we found that only 13-22% of the cell type-specific differentially methylated regions are covered by the Illumina 450K and EPIC DNA methylation arrays, emphasizing the benefits of a whole-genome sequencing approach for exhaustive identification of biomarkers.

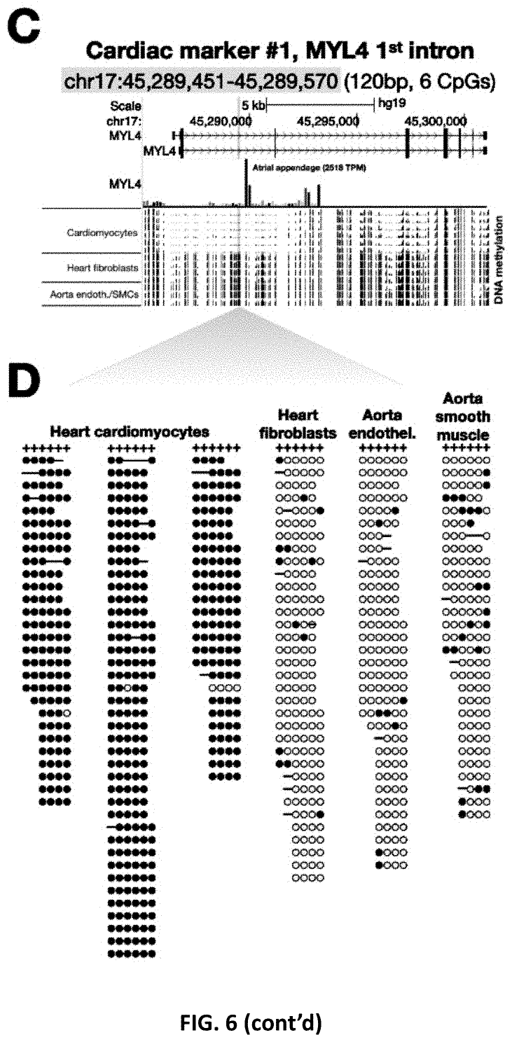

To obtain a human cell type-specific methylation atlas, we identified the top 25 (top markers) and top 125 (extended markers) differentially unmethylated regions, and top 25 differentially methylated regions, for each cell type (sequence listing). As FIG. 6 shows (for the top 25 unmethylated markers), these regions are uniquely demethylated in particular cell types and are methylated in all other samples, and can serve as sensitive biomarkers for identifying and quantifying the presence of DNA from a specific cell type in a mixture. This approach has various applications, including the analysis of cell-free DNA fragments circulating in the blood.

### Cell Type-Specific Unmethylated Regions are Tissue-Specific Enhancers

We next turned to characterize the sets of cell type-specific differentially unmethylated regions. Using GREAT, we identified the adjacent genes of each group of cell type-specific markers, and tested them for enrichment of various gene-set annotations. The genes adjacent to loci uniquely unmethylated in a given cell type typically reflected the functional identity of this cell type. For example, B cell methylation markers were enriched near genes associated with B cell morphology, B cell differentiation, B cell number, IgM levels, and lymphopoiesis; NK cell markers associated with gene sets related to NK cell mediated cytotoxicity, hematopoietic system, cytotoxicity, and lymphocyte physiology; T cell markers were associated with gene sets linked to the number, activation status, differentiation, physiology and proliferation of T cells; Fallopian tube markers were enriched for genes related to egg coat and perivitelline space; and cardiomyocyte markers were enriched for genes related to cardiac relaxation, systolic pressure, muscle development, and hypertrophy.

We then analyzed the chromatin packaging of the genome regions that surround cell type-specific methylation markers. We focused on DNA accessibility, via published ATAC-seq and DNaseI-seq datasets, as well as histone marks indicative of active gene regulation at promoters and enhancers, via ChIP-seq data for H3K27ac and H3K4mel. The top 250 cell type-specific DNA unmethylated markers for monocytes and macrophages are characterized by high H3K27ac and H3K4mel in monocytes, as well as high DNA accessibility. Conversely, markers of other blood cell types show no such enrichment in monocytes. We also calculated the positional enrichment of enhancer state near these cell type-specific markers, as annotated by chromHMM in matching cell types. These findings are consistent with previous studies that have associated tissue-specific demethylation with gene enhancers.

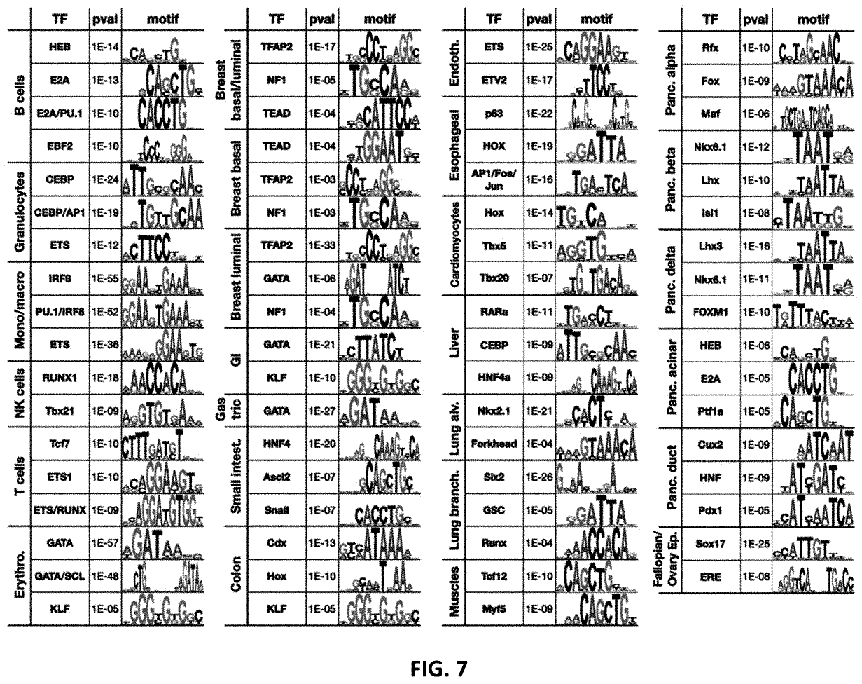

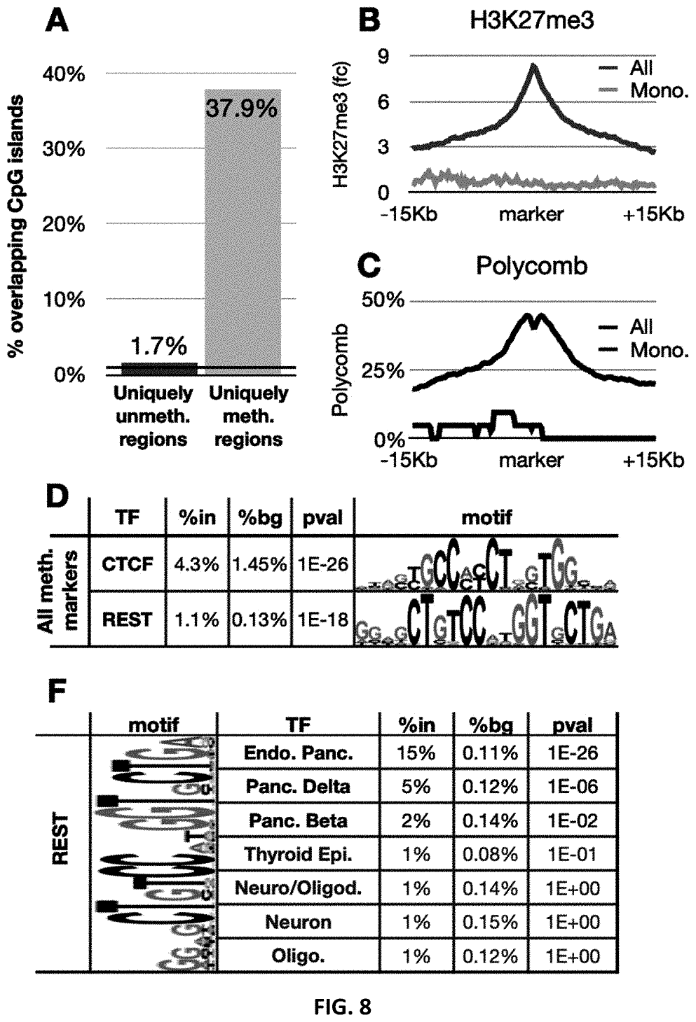

To further assess the biological importance of cell type-specific unmethylated regions, we studied their association with transcription factors that could affect DNA methylation, or bind DNA in a cell type-specific manner, depending on methylation and chromatin. We performed a motif analysis using HOMER, and calculated the enrichment (within the unmethylated markers of each cell type) for known transcription factor binding site motifs. For most cell types, the most significant motifs included master regulators and key transcription factors that govern their transcriptional program (FIG. 7). For example HEB/Ebf2/E2A/PU.1 for B cells, CEBP/AP1/ETS for granulocytes, Tcf7/ETS/RUNX for T cells, GATA/SCL/KLF motifs for erythrocyte progenitors, and GATA/KLF/HNF/Asc12/Cdx motifs for gastrointestinal (GI) epithelial cells. We propose that the association between cell type-specific demethylated regions and transcription factor binding motifs can be used to identify novel gene regulatory circuits that operate by providing transcription factor access to specific enhancers in specific cell types.

### Identification of Target Genes Regulated by Cell-Type Specific Enhancers

We attempted to identify the target genes of the putative enhancers marked by cell type-specific lack of methylation. Some of the top 25 markers fall within intronic regions and are likely to regulate these same genes (for example glucagon in pancreatic alpha cells; NPPA, MYH6, and MYL4 in cardiomyocytes, or EPCAM in GI epithelial cells), while some of the top markers are proximal to possible targets (e.g., a beta cell marker 5 Kb from the Insulin gene). Yet other markers are further apart, and we devised a computational algorithm that integrates the distance between each cell type-specific marker and surrounding genes, as well as the expression patterns of these genes. Specifically, we aimed for genes that are expressed in the same cell types where the marker is unmethylated, compared to other cell types where the marker is methylated. We applied an iterative bidirectional z-score calculation, where the over-expression of a gene in a given condition is compared to its expression in other conditions, and the expression of genes in the condition. This highlighted hallmark genes for many cell types, and allowed us to associate a putative target gene for many of the top 25 unmethylated markers for each cell type. For example, hepatocyte markers were associated with APOE, APOC1, APOC2, Alpha 2-antiplasmin, and the glucagon receptor (GCGR). Similarly, cardiomyocyte markers were associated with NPPA, NPPB, and myosin genes; and pancreatic islet markers with the insulin and glucagon genes. These findings further support the principle that loci specifically unmethylated in a given cell type are likely enhancers positively regulating genes expressed in this cell type, often controlling adjacent genes. We note however that very often, the genes adjacent to a locus specifically unmethylated in a given cell type are broadly expressed beyond this cell type (see discussion).

Cell Type-Specific Hyper-Methylated Regions are Enriched for CpG Islands and for Polycomb. CTCF, and REST Targets

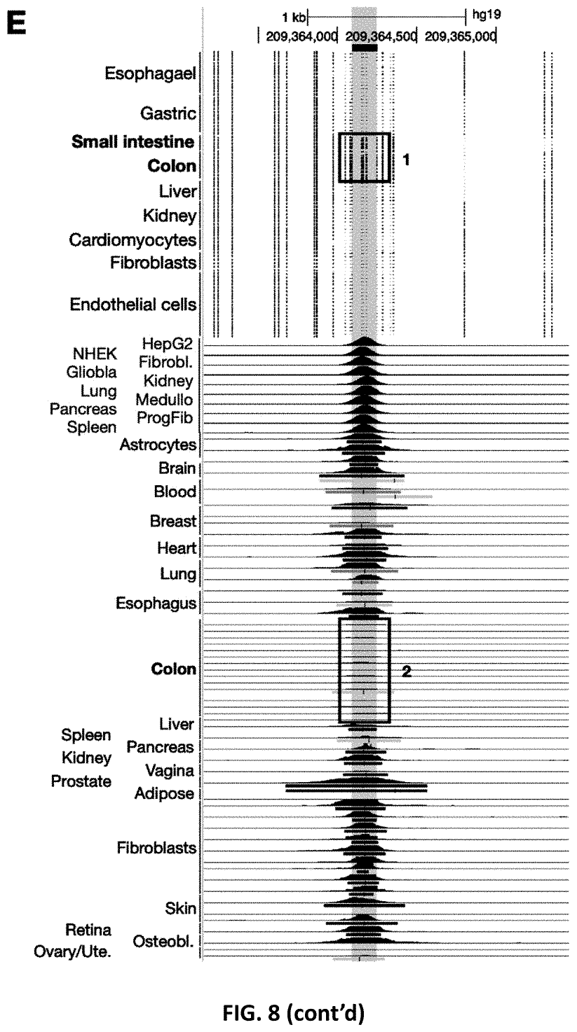

Finally, we studied the genomic regions that are methylated in one cell type, but unmethylated elsewhere in the human body. These are enriched for CpG islands (38% of methylated regions, compared to 1.7-2.7% of cell type-specific unmethylated regions), and are marked by H3K27me3 and polycomb in other cell types (FIG. 8A-C), as previously reported for cancer and developmental processes. These cell type-specific hyper-methylation regions were generally less significant for motif enrichment (compared to uniquely unmethylated regions), possibly due to their smaller number. Intriguingly, only ˜3% of the total set of cell type-specific differentially methylated regions are hyper-methylated.

However, when we pooled together all cell type-specific hyper-methylated regions, we identified a strong enrichment for the target sequences of the chromatin regulator CTCF (p≤1E-26) (FIG. 8D). This suggests that DNA methylation of CTCF binding sites could act as a tissue-specific regulatory switch to modulate its binding, potentially affecting tissue-specific 3D genomic organization. To test this idea, we compared patterns of DNA methylation at CTCF sites with data on genome-wide CTCF protein binding in specific tissues. FIG. 8E shows the methylation pattern and the published in vivo CTCF occupancy at one locus, which is methylated specifically in the colon and intestine. Consistent with DNA methylation preventing CTCF binding, ChIP data show selective absence of CTCF binding at this locus in the colon. In addition, loci methylated in specific cell types were enriched for targets of the transcriptional repressor of neural genes, REST/NRSF (p≤1E-18), and this was seen most prominently in the methylome of pancreatic islet cells (FIG. 8F). While DNA methylation has not been shown to affect the binding or activity of REST, this finding raises the possibility that methylation of REST targets in a specific tissue could endow this tissue with the ability to differentiate independently of REST repression.

The comprehensive atlas of human cell type methylomes described here sheds light on principles of DNA methylation, and provides a valuable resource for multiple lines of investigation, as well as translational applications.

### Variation of DNA Methylation Between Replicates and Different Cell Types

Our analysis revealed that methylation patterns are strikingly similar among healthy biological replicates of the same cell type from different individuals. From a practical perspective, this suggests that a small number of samples are sufficient to determine the methylation blueprint of any given cell type. From a developmental biology perspective, the similarity between individuals reflects the extreme robustness of cell differentiation and maintenance circuits, at least as far as healthy tissues are concerned. Pathologies involving destabilization of the epigenome obviously disrupt these circuits, resulting in a much larger variety of methylation patterns among cells that descend from a specific normal cell type. We predict that even in cancer (when examining tumors of the same primary anatomic site and histologic type), comparative methylome analysis of epithelial cells (free of stroma), performed at the level of methylation blocks, will reveal a smaller inter-individual variation than typically assumed.

As the atlas blocks revealed, each cell type has a set of genomic regions that are specifically and uniquely unmethylated in that cell type compared to others, as well as additional genomic regions that share methylation patterns with related cell types. An unsupervised clustering of cell type-specific methylomes revealed similarities between cell types that could not be explained by common gene expression patterns. Instead, cell types in the atlas were clustered in ways that reflected their developmental origins. This was most apparent in the methylation-based similarity between beta cells and cells of the exocrine pancreas and the liver, which share endodermal origins but have little in common with regard to function; this similarity was in stark contrast to the distance of beta cell methylation from that of neurons, which share common function but derive from a different lineage. This offers a fascinating view of DNA methylation as a living record of the methylomes of progenitor cells, retained in the genome through dramatic embryonic developmental transitions and decades of life thereafter. Perhaps the most striking example of this principle is the clustering of cells according to their germ layer of origin. The loci that drive the clustering of colon epithelial cells from one adult donor with lung alveolar cells of another donor are probably reflecting the common origins of these cell types in the embryonic endoderm, which forms during gastrulation and diverges shortly after. We propose that comparative methylome analysis will allow reconstructing parts of the methylomes of fetal structures or cell types, similarly to the reconstruction of last common ancestors in evolutionary biology.

### Cell Type-Specific Demethylation Identifies Enhancers and TF Binding Motifs

The vast majority of the cell type-specific differentially methylated regions were specifically demethylated in one cell type, suggesting a positive regulatory role for that region. Indeed, an unbiased analysis of the chromatin packaging of these genomic regions across a variety of cells revealed that they are typically highly accessible and bear histone marks associated with active gene regulation, as found in enhancers and promoters. Moreover, a motif analysis for these genomic regions identified a statistically significant enrichment of transcription factor binding site motifs, and deciphered much of the regulatory circuitry for each cell type. Finally, we devised an integrated approach that, based on distance and gene expression profiles, allowed us to highlight possible target genes for these putative enhancer regions. Notably, many enhancer regions were associated with nearby genes that are broadly expressed, potentially reflecting gene regulation by multiple tissue-specific enhancers.

In this example we used strict definitions of cell type-specificity and focused on genomic regions that are uniquely unmethylated in a given cell type, compared to all others. Obviously, the DNA methylation atlas permits different analytical approaches. A more lenient definition of specificity will reveal tens of thousands of additional putative enhancers per cell type.

### Roles for Cell Type-Specfic Hyper-Methylation

Conversely, we identified genomic regions that are specifically methylated in one or two cell types but unmethylated in all other atlas cell types. These regions represent about ˜3% of cell type-specific differentially methylated regions. They are often located in CpG islands, and characterized by H3K27me3 and polycomb binding in tissues where the locus is not methylated. These regions are significantly enriched for CTCF binding sites, suggesting a role for DNA methylation in attenuating the binding of CTCF and thus modulating the 3D organization of neighboring DNA, including enhancers and their target genes. The specifically methylated regions also showed enrichment for the transcriptional repressor REST/NRSF binding site motif, suggesting yet another role for DNA hyper-methylation in prevention of REST binding and gene repression in some cell types. Of particular interest is the enrichment of the REST/NRSF motif in blocks that are methylated in pancreatic islet cells. REST represses neuronal differentiation in non-neural tissues, and endocrine differentiation in the fetal and exocrine pancreas. We believe that methylation of REST targets in the endocrine pancreas serves to guarantee protection of islet genes from accidental repression by REST.

### Cell Type-Specific DNA Methylation Biomarkers for Cell-Free DNA Analysis

The atlas described here is the most comprehensive whole-genome healthy DNA methylation atlas to date. We have identified over a thousand cell type-unique DNA methylation regions that could serve as accurate and highly specific biomarkers for identifying and quantifying cell death events by monitoring cell-free DNA fragments circulating in the blood. Notably, the vast majority of these marker regions are not covered by the 450K/EPIC BeadChip DNA methylation arrays, and were not previously appreciated. The resolution of the atlas yields a quantitative understanding of composite tissues, and allows one to identify missing methylomes of additional cell types that are yet to be sorted and characterized.

In summary, this example presents a comprehensive methylome atlas of primary human cell types and provide examples for biological insights that can be gleaned from this resource. Among the many potential utilities of this atlas, perhaps most promising is the possibility to use it for deconvolution of cell types in a mixed cell type sample, and sensitive identification of the tissue of origin of cfDNA in plasma of individuals with cancer and other diseases.

### Example 2. Universal Lung Epithelium DNA Methylation Markers for Detection of Lung Damage in Liquid Biopsies

Liquid biopsies using circulating cell-free DNA (cfDNA) are extensively used for monitoring patients with lung cancer. Analysis of cfDNA molecules carrying oncogenic mutations allows to assess disease progression, response to therapy, and evolutionary dynamics in the cancer genome. The strength of this approach—the ability to assess the tumor genome via a blood test—is also the source of its inherent limitations. It requires personalization of analysis for the mutations of each particular tumor; it is blind to tumors in which the mutational profile is not known, and to the dynamics of tumor clones not containing the mutation(s) being studied; and it cannot identify the tissue source of malignancy (for example, whether a lesion in the lung represents lung cancer or metastasis from a different site). Most fundamentally, liquid biopsies that rely on somatic mutations are blind to pathologies that involve damage to lung cells with a normal genome, including cancer-induced collateral damage to adjacent epithelial cells.

Liquid biopsies using lung-specific methylation markers can theoretically offer a universal circulating lung biomarker, applicable to cfDNA derived from all lung lesions in all individuals. Such a biomarker is expected to be highly specific, due to the cell-type specificity of DNA methylation. Theoretically, the sensitivity of tissue-specific methylation markers can be enhanced by parallel assessment of multiple informative genomic loci in the same plasma sample, with a minimal loss of specificity.

Example 1 has developed a method for targeted analysis of cell type-specific methylation markers. To identify such markers for lung epithelial cells, this example now determined the methylomes of sorted alveolar and bronchial epithelial cells and compared them to an extensive methylome atlas of other human tissues. The analysis revealed hundreds of loci that are uniquely methylated or unmethylated in lung epithelial cells, representing the epigenetic basis for the cellular identity and gene expression program of lung epithelium, including differences between alveolar and bronchial compartments. The maps also allowed the development of a panel of lung-specific methylation markers.

This example reports the analysis of lung epithelial methylomes, and characterization of a universal lung marker panel. As proof of concept, we applied the markers for the assessment of lung-derived DNA in plasma from healthy individuals, patients with lung cancer, individuals undergoing bronchoscopy and patients with COPD.

### Materials and Methods

Patients. All clinical studies were approved by the ethics committees of Hadassah and Shaare Zedek Medical Center.

Biomarkers. Tissue-specific methylation biomarkers were selected after a comparison of publicly available genome-wide DNA methylation datasets generated using Illumina Infinium HumanMethylation450k BeadChip array. The comparison included in addition the methylome of human alveolar and bronchial epithelial cells, generated by whole genome bisulfite sequencing from sorted dissociated Lung tissue. Table 3 lists the coordinates of markers, and primers used to amplify them.

TABLE 3
Listing of Markers

| Marker type | Chromosome | Location | Ref Gene | Meth |
|---|---|---|---|---|
| Lung 1 | 14 | 55765534 | FBX034 | U |
| Lung 2 | 3 | 181441571 | SOX2OT | M |
| Alveo1 | 1 | 41486102 | SLFNL1 | U |
| Alveo2 | 2 | 236672684 | AGAPI | U |
| Alveo3 | 17 | 79952367 | ASPSCR1 | U |
| Alveo4 | 16 | 678127 | RAB40C | U |
| Alveo5 | 7 | 2473529 | CHST12 | U |
| Alveo6 | 16 | 1652552 | IFT140 | U |
| Alveo7 | 14 | 91691190 | C14orf159 | U |
| Alveo8 | 16 | 667157 | RAB40C | U |
| Alveo9 | 11 | 66116455 | B3GNT1 | U |
| Alveo10 | 4 | 57522145 | HOPX | M |
| Alveo11 | 16 | 84271391 | KCNG4 | U |
| Alveo12 | 1 | 1986275 | PRKCZ | U |
| Bronch1 | 7 | 4802132 | FOXK1 | U |
| Bronch2 | 2 | 239970075 | HDAC4 | U |
| Bronch3 | 1 | 164761834 | PBX1 | U |

Sample Preparation and DNA Processing. Blood samples were collected in plasma-preparation tubes and centrifuged for 10 min in 4 degrees at 1,500×g. The supernatant was transferred to a fresh 15 ml conical tube without disturbing the cellular layer and centrifuged again for 10 min in 4 degrees at 3000×g. The supernatant was collected and stored in −80c.

Cell-free DNA was extracted from 1-4 mL of plasma using the QIAsymphony liquid handling robot (Qiagen) and treated with bisulfite (Zymo Research). DNA concentration was measured using Qubit High Sensitive double-strand molecular probes (Invitrogen). Bisulfite-treated DNA was PCR amplified using primers specific for bisulfite-treated DNA but independent of methylation status at monitored CpG sites.

Primers were bar-coded, allowing the mixing of samples from different individuals when sequencing products. We used a multiplex 2-step PCR protocol. Sequencing was performed on PCR products using MiSeq Reagent Kit v2 (MiSeq, Illumina method) or NextSeq 500/550 v2 sequencing reagent kits. Sequenced reads were separated by barcode, aligned to the target sequence, and analyzed using custom scripts written and implemented in Matlab. Reads were quality filtered based on Illumina quality scores. Reads were identified by having at least 80%/o similarity to target sequences and containing all the expected CpGs in the sequence. CpGs were considered methylated if “CG” was read and were considered unmethylated if “TG” was read. The efficiency of bisulfite conversion was assessed by analyzing the methylation of non-CpG cytosines.

Lung epithelium sorting. Fresh surgical samples of alveolar and bronchial lung were dissociated. Alveolar and bronchial epithelial isolated cells were sorted by FACs using CD45 eFluor 450, CD31 eFluor 450 and CD234A eFluor 450 (eBioscience) and CD326 (Miltenyi) antibodies.

### Results

### Methylomes of Alveolar and Bronchial Epithelial Cells

Previous studies have characterized the methylomes of unsorted or laser-captured lung tissue, using either Illumina BeadChip arrays (which cover up to 3% of the genome) or shallow whole genome bisulfite sequencing. Such preparations typically contain mixtures of lung epithelial cells and other cell types (e.g., endothelial cells, pericytes, fibroblasts, blood cells). Importantly, non-epithelial cell types vary in their proportion in lung tissue and can even constitute the majority population, complicating the extraction of lung epithelium-specific methylation markers from such datasets. To overcome this limitation, we sorted ultra-pure populations of lung epithelial cells from fresh surgical material, using an antibody against the Epithelial Cell Adhesion Molecule (EpCAM) as a cell surface marker. The starting material was a piece of lung tissue, from a bronchial area or from a distal alveolar area. The tissue fragments were typically obtained during surgery for removal of lung cancer, from an area as far as possible from the tumor. We dissociated the tissue to single cells, stained for EpCAM, sorted EpCAM+ and EpCAM-cells using flow cytometry and prepared RNA and genomic DNA from sorted cells. Quantitative RT-PCR for EpCAM confirmed that the EpCAM+ population was indeed highly enriched for epithelial cells. We then subjected genomic DNA from bronchial epithelial cells (n=3 donors) and from alveolar epithelial cells (n=3 donors) to whole-genome bisulfite sequencing, with an average coverage of 30×.

Analysis of the resulting methylomes revealed a high similarity between preparations of the same cell type from different individuals, consistent with the conserved nature of cell-type specific methylomes, and supporting the conclusion that the preparations represented highly purified epithelial cells (FIG. 9A).

We compared the lung alveolar and bronchial methylomes to an extensive atlas of human cell type methylomes, containing >200 methylomes and representing 40 different cell types, and identified differentially methylated regions that are uniquely methylated or unmethylated in either lung cell type, compared with all other cell types. Consistent with the different functions of bronchial and alveolar cells, the similarity between their methylomes was limited: Out of ˜2400 regions that are differentially methylated in the lung compared with other tissues, only ˜140 loci (5.8%) were shared among bronchial and alveolar cells (FIG. 9A). We subjected the methylomes to an unsupervised hierarchical clustering, and found that the samples from each of the two cell types tightly cluster within themselves. Interestingly, the bronchial methylomes clustered together with methylomes of larynx epithelial cells, suggesting that bronchial and larynx epithelial cells are more similar to each other than to lung alveolar cells. We also observed an intriguing similarity between the methylomes of lung cells and the methylomes of bladder and prostate epithelial cells. In addition, hundreds of loci were uniquely methylated or unmethylated in lung alveolar (˜1,600 loci) or bronchial (˜700 loci) epithelial cells, compared with all other tissues in the atlas, apparently underlying the epigenetic basis for unique gene expression programs of these cell types. All together, we identified about 2,500 loci that have a lung-specific methylation pattern, mostly unmethylated in lung epithelium and methylated elsewhere. Initial computational analysis of loci specifically unmethylated in lung epithelial cells revealed enrichment for gene promoters and enhancers, overlapping regulatory histone marks such as histone H3 lysine 27 acetylation (H3K27ac), and the enhancer mark histone H3 lysine 4 monomethylation (H3K4mel). Lung unmethylated regions are also enriched 3-10 fold for the presence of enhancers based on genome-wide annotation of lung chromatin (FIG. 9B-C).

Next, we used GREAT to associate differentially methylated regions with nearby genes and identify enrichment for specific biological functions. Genomic regions specifically unmethylated in either alveolar or bronchial epithelial cells were enriched near gene sets that relate to lung biology, indicating that at least some of the loci specifically unmethylated in lung epithelium are promoters or proximal enhancers of lung-specific genes (FIG. 1D). Some examples of genes that reside immediately adjacent to loci with lung-specific hypomethylation include lung transcriptional regulators Eya1 and Nkx2.1, and the surfactant B gene SFTPB. However, most genes adjacent to loci unmethylated in lung were expressed in multiple tissues other than the lung, suggesting that the unmethylated loci represent either a distal enhancer of another gene, or a lung-specific enhancer of a gene with broad expression.

Finally, we used the bronchial and alveolar methylomes, along with other methylomes in our atlas, to deconvolute previously-published methylomes of alveolar and bronchial tissue, obtained by laser capture microscopy. This analysis revealed that the published alveolar methylomes contained 20-30% alveolar DNA, mixed with DNA of vascular endothelial cells, fibroblasts and blood cells. The published bronchial methylomes had varied contribution of bronchial DNA, with ˜50% of the DNA in fact derived from alveolar cells. These findings highlight the value of sorted cells for obtaining lung epithelial cell methylomes.

### Characterization of Lung-Specific Methylation Markers

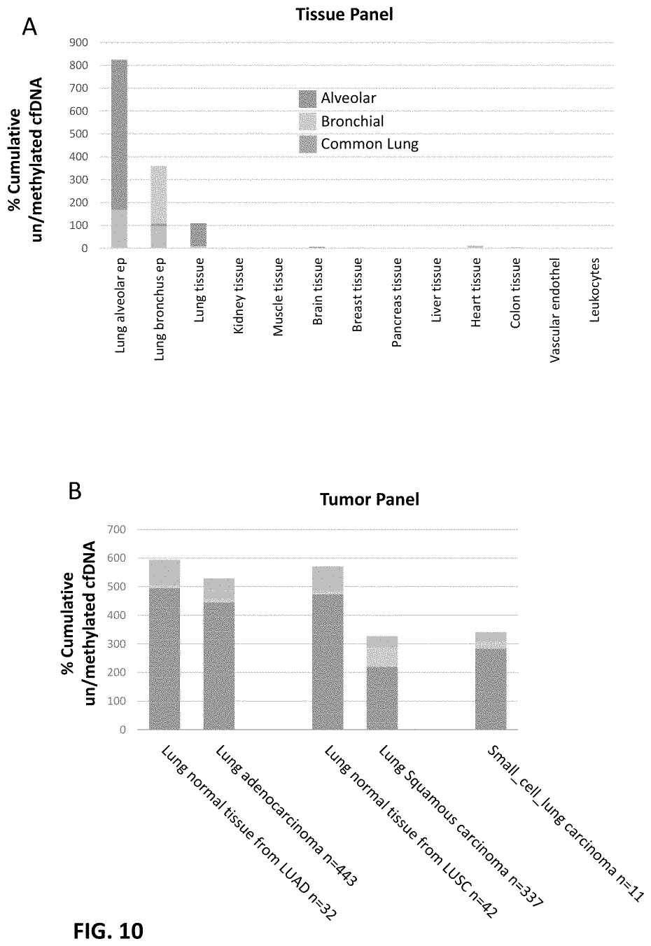

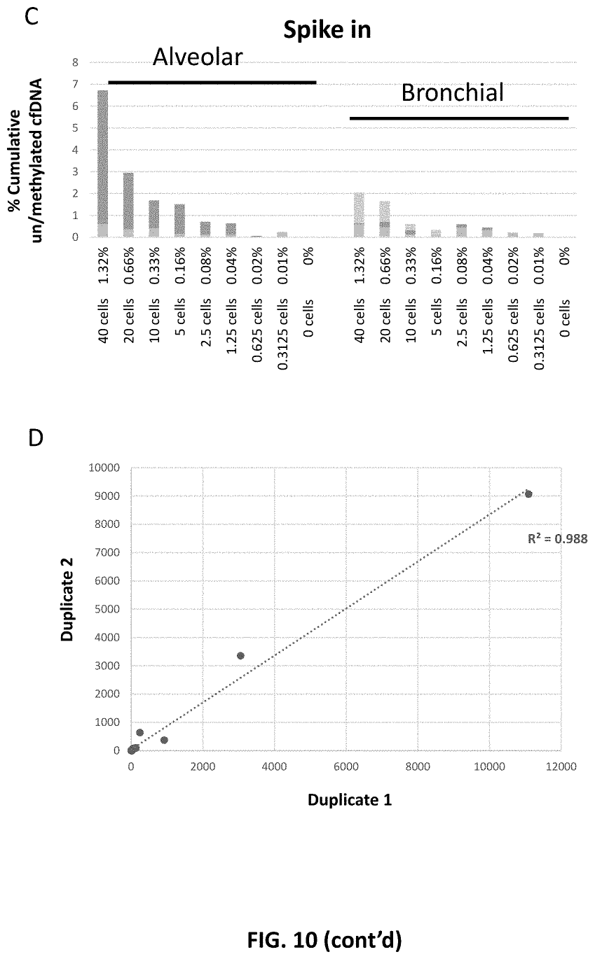

To generate methylation markers for targeted cfDNA analysis, we selected 17 genomic loci (Table 3) that were uniquely unmethylated or hypermethylated in lung epithelial cells, including loci that identify specifically bronchial cells (n=3) alveolar cells (n=12) or both types of lung epithelial cells (n=2), and prepared PCR primers to amplify these loci in two multiplex PCR reactions after bisulfite conversion (see methods). Sequencing the PCR products that were amplified from a panel of tissues and cell types confirmed the extreme specificity of alveolar, bronchial and general lung epithelium methylation markers (FIG. 10A). We also examined the status of these markers in hundreds of lung cancer methylomes, available through TCGA. Lung cancers retained the methylation patterns of common lung markers. Lung adenocarcinoma DNA had alveolar but not bronchial methylation markers, while lung squamous carcinoma contained both alveolar and bronchial markers, consistent with the presumed tissue origins of these tumors (FIG. 10B). These findings support the relevance of our universal lung markers for lung cancer analysis.

To assess the ability of markers to identify rare lung DNA when present within a large excess of non-lung DNA, we spiked different amounts of alveolar or bronchial DNA into leukocyte DNA and assessed the fraction of lung DNA using the methylation assay. Lung DNA could be identified when it contributed as little as 0.04% of the DNA in a mixture, or when there were only 1.25 lung genome equivalents in the mixtures (FIG. 10C). Finally, to assess the reproducibility of the assay we ran 19 plasma samples in duplicates or triplicates, and found an excellent correlation (FIG. 10D).

These data establish a cocktail of methylation markers that can identify lung epithelial DNA from essentially any human donor with extreme sensitivity, and specificity that is retained even in lung cancer.

### Lung-Derived DNA in Healthy Donors

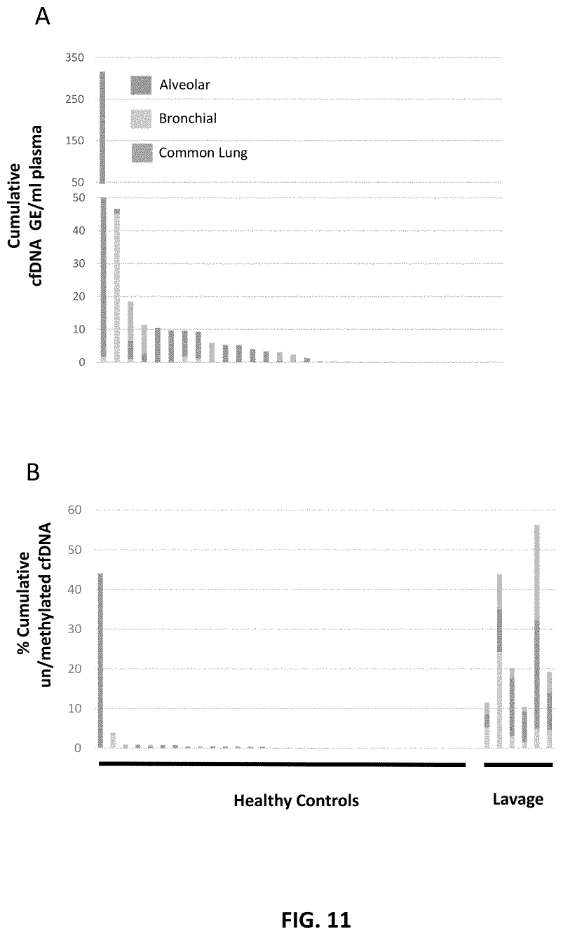

Epithelial cells in the lung turn over at an estimated rate of 0.83% per day. Given that the number of epithelial cells in the human lung is ˜10¹¹, about 10⁹ cells die each day. The DNA of such dying cells could in principle be eliminated locally by phagocytes, released to blood, or released to the air spaces of the lung. To distinguish between these possibilities we measured the presence of lung DNA in plasma samples from 30 healthy individuals. Most samples had no DNA molecules carrying the methylation signature of lung epithelium, with the exception of one individual that had 3.7% of cfDNA derived from the lung (31 GE/ml, calculated as the average value for all lung markers), and one individual that had 0.25% of cfDNA derived from the lung (0.83 GE/ml). Both donors did not have obvious medical conditions that could explain the high levels of lung cfDNA (FIG. 11A-B). We then obtained material from a broncho-alveolar lavage (BAL), a procedure whereby a lobe of the lung is washed with a large volume of saline. We extracted DNA from the BAL fluid of individuals that underwent the procedure for suspicion of cancer or other pathologies, but turned out to have either no pathology, or mild pneumonitis. The BAL DNA from 6 out of 6 donors contained lung DNA including both alveolar, bronchial and general lung markers. On average, 2.98% of BAL DNA was derived from lung epithelium. The rest of BAL DNA was derived from immune cells.

These findings indicate that under normal conditions, dying lung cells release DNA fragments to the air spaces but not to blood. We propose that this situation reflects lung topology, which dictates the route of clearance of material from dying. This is similar to what is observed in the intestine, where material from dying cells during normal turnover reaches the lumen of gut rather than the blood.

Lung-Derived cfDNA in Patients with Advanced Lung Cancer

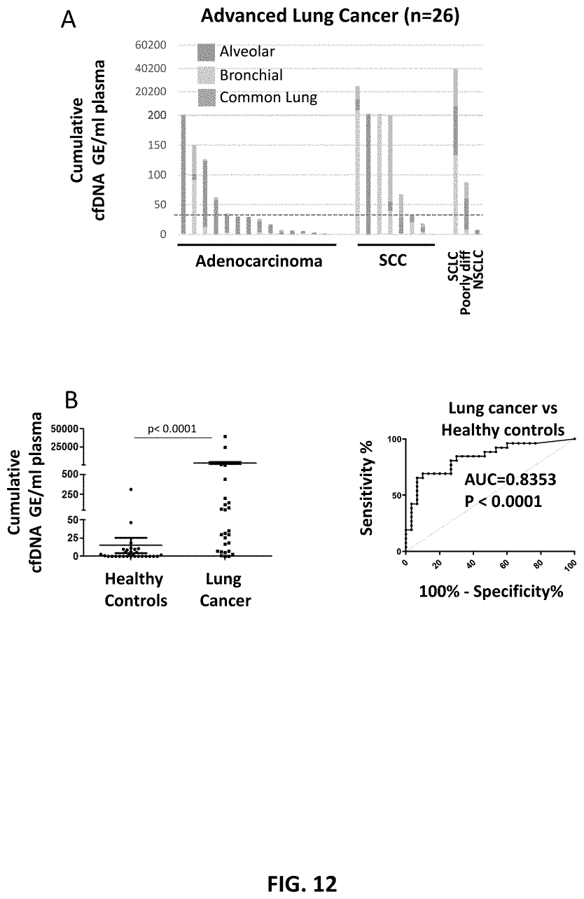

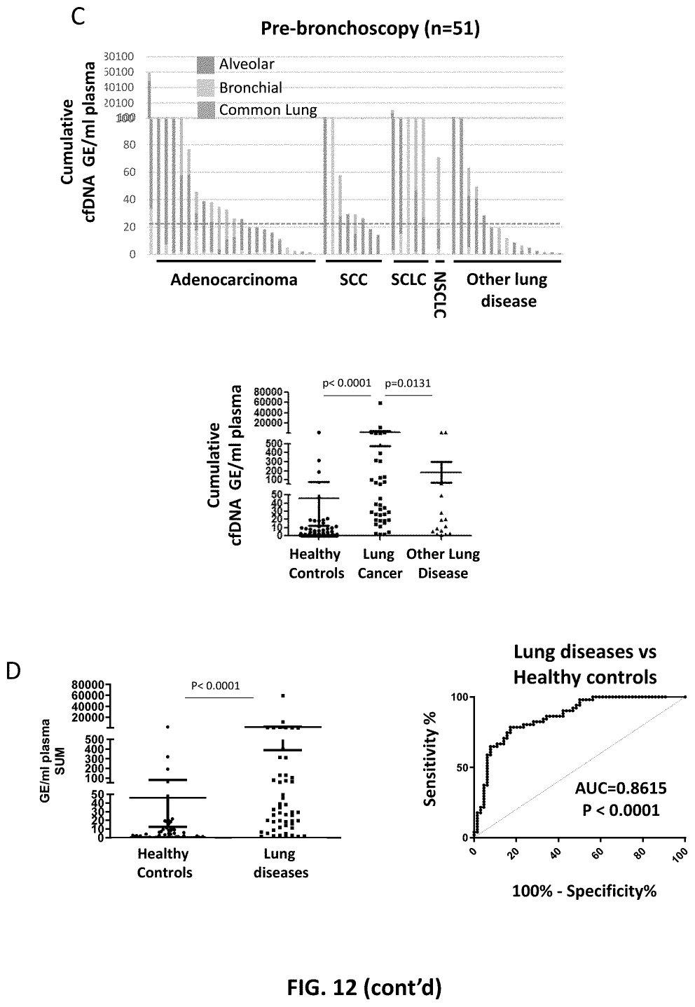

Having defined the extremely low levels of lung cfDNA in the plasma of healthy donors, we next assessed the levels of lung-derived cfDNA in the plasma of lung cancer patients, using the same cocktail of normal lung epithelial cell markers. We used 26 samples from patients with advanced lung cancer including adenocarcinoma, squamous cell carcinoma (SCC), small cell carcinoma (SCLC) and Poorly Differentiated Carcinoma. The patients had varying tumor burdens and were mostly under treatment. The average concentration of normal lung cfDNA in these patients was 36 GE/ml plasma (p<0.0001 compared with healthy donors) (FIG. 12A-B), and a receiver operating characteristic (ROC) curve was able to distinguish healthy from cancer plasma with an area under the curve (AUC) of 0.835 (FIG. 12B).

While this was a small cohort intended for a proof of concept showing presence of normal lung methylation markers in the plasma of cancer patients, we observed an interesting link between the specific lung markers observed and the presumed tissue of origin of cancer. cfDNA from patients with adenocarcinoma, thought to derive from type 2 pneumocytes residing in the alveoli, showed mostly alveolar markers. In contrast, samples from patients with SCC, thought to derive from bronchi, had a stronger representation of bronchial cfDNA markers (FIG. 12A). Nonetheless, all marker classes—alveolar, bronchial, and common—have contributed to the signal observed in the plasma of cancer patients.

Lung-Derived cfDNA in Patients Undergoing Bronchoscopy

An important test for a cfDNA biomarker is its ability to identify pathology prior to obtaining definitive knowledge from other sources. To perform such a test, we established a prospective cohort of individuals that were referred to bronchoscopy for suspicion of cancer. We obtained plasma samples from 51 individuals just prior to bronchoscopy, prepared cfDNA and assessed the presence of lung-derived cfDNA blindly. We then compared the results to the outcome of histopathological analysis of the bronchoscopy reported later. As shown in FIG. 12C, abnormally high levels of lung cfDNA were identified in 25 out of 36 patients diagnosed as having lung cancer. As expected, some patients with other lung pathologies also had elevated lung cfDNA (5 out of 15). Overall, the plasma of bronchoscopy patients with any lung disease had significantly higher levels of lung cfDNA than healthy individuals (FIG. 12D). The ROC curve for distinguishing patients with lung pathology from healthy individuals based on lung cfDNA had an AUC of 0.8615 (FIG. 12D, right panel). At 70% specificity, lung cfDNA had an 82.35% sensitivity for detection of lung diseases among healthy individuals (see below).

### The Value of Multiplexing Markers

Multiplexing cfDNA markers—that is, assessing the presence of multiple independent biomarkers in the same plasma sample—is seen as a promising approach for sensitizing liquid biopsies to allow for early detection of disease. The abundance of universal DNA methylation markers for any given tissue permits in principle to multiplex and hence sensitize methylation-based cfDNA markers, potentially beyond what is afforded by mutation-based analysis. We took advantage of our lung-specific methylation cocktail to assess empirically whether the use of additional markers increases the likelihood of identifying lung-derived cfDNA in patients with lung pathology, without compromising specificity. As shown in FIG. 13, the best methylation marker produced an AUC of 0.75 for distinguishing the plasma of patients with any lung disease from the plasma of healthy people. Adding additional markers further increased the AUC, with a combination of 17 markers providing improved sensitivity over the combination of 3 markers (FIG. 13).

Lung cfDNA in Patients with COPD

We next assessed the presence of lung-derived cfDNA in the plasma of patients with COPD, a lung disease for which there are currently no circulating biomarkers. This is interesting and challenging for several reasons. First, lung epithelium is not mutated in COPD, precluding the use of somatic mutations as biomarkers. Second, it is not clear if lung damage in COPD is sufficient to reverse tissue topology and release cfDNA to blood rather than to the air spaces. For example, in Crohn's disease we found that the damage to intestinal epithelial cells does not lead to cfDNA release to plasma.

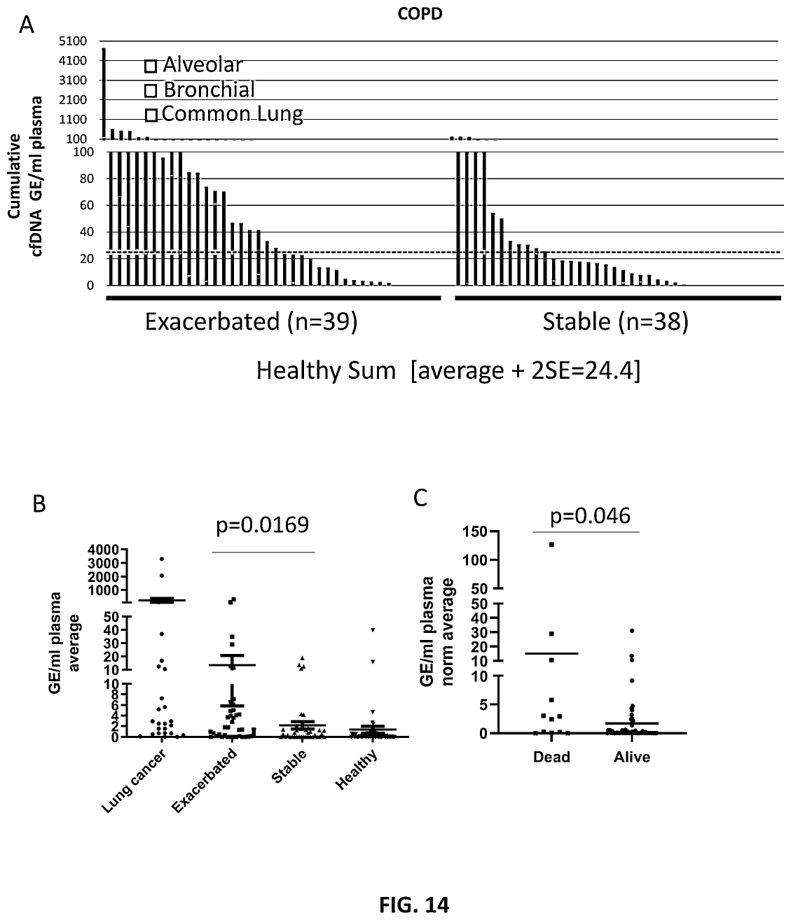

We obtained 77 plasma samples from patients with exacerbated (n=39) or stable (N=38) COPD, and determined blindly the levels of general and lung-specific cfDNA in these samples. Patients with exacerbated COPD had significantly more lung-specific cfDNA than patients with a stable disease, but less than patients with advanced lung cancer (FIG. 14A-B). We assessed multiple parameters that could potentially underlie the difference in lung cfDNA levels between patients with exacerbated and stable disease. Lung cfDNA did not correlate with patient age, gender, smoking habits, and the presence of emphysema (Table 4). This suggests that lung cfDNA truly reflects the severity of lung disease. In support of this idea, COPD patients who have died up to 14 months after sampling (n=12) had significantly higher levels of lung cfDNA at the time of sampling (FIG. 14C).

TABLE 4
Correlations between king cfDNA and clinical/
demographic parameters among COPD patients

| | p value test |
|---|---|
| | (relative to |
| | cfDNA sum |
| Condition | ad markers) |
| Exacerbated vs stable | 0.018 |
| FEV1 LITERS | 0.019 |
| Future exacerbation vs none | 0.0255 |
| Dead vs Alive | 0.0365 |
| Age | 0.22 |
| Emphysema | 0.115 |
| Male vs Female | 0.1555 |
| FEV1 Per | 0.367 |
| Smoker vs non-smoker | 0.376 |

Our analysis of the methylomes of human alveolar and bronchial epithelial cells led to several insights. First, whole genome bisulfite sequencing of highly purified epithelial cells, sorted from primary surgical preparations, revealed the complete methylation landscape of lung epithelial cells, which comprise only a minority population in mixed preparations of previously reported lung tissue. Second, a global comparison of lung epithelial cell methylomes to other cell type-specific methylomes revealed that alveolar and bronchial epithelial cells are highly divergent. In fact, bronchial epithelial cells are more similar to epithelial cells of the larynx than to alveolar cells, reflecting their common origin as well as function in conductance of air. The divergence of alveolar cell methylomes from bronchial cells likely reflects the complex differentiation pathway of terminal branching morphogenesis in the lung. Third, most loci showing a lung-specific methylation pattern are unmethylated in lung epithelial cells and methylated elsewhere, and are enriched for lung-specific gene enhancers. The finding that lung-specific methylation markers are typically lung-specific enhancers is consistent with previous studies in other systems, for example our previous demonstration that pancreatic beta cell methylation markers are enriched for beta cell-specific gene enhancers. Interestingly, the genes that are closest to lung-specific methylation markers are enriched for lung-related gene sets, even though the expression of individual genes in these gene sets is typically not restricted to the lung. These findings suggest that lung-specific enhancers (demethylated in lung cells) regulate lung expression of genes, while other enhancers control the expression of these genes in other tissues.

The detailed analysis of lung methylomes allowed the identification of specific loci that can serve as markers for identification of lung DNA in a mixture. Importantly, our lung-specific markers retain their typical methylation pattern in lung cancer, suggesting utility as universal biomarkers.

How tissues clear debris from dying cells is an important yet neglected aspect of tissue topology and dynamics. In the case of normal lung epithelium, genomic DNA from dead epithelial cells could in principle be released to blood (as in the case of the liver), or to the air spaces (analogous to the release of DNA from intestinal epithelial cells to the lumen of the gut). Our data strongly support the latter possibility, that is the release of DNA from normal lung during tear and wear into the air spaces, where it is likely digested by lung macrophages. This arrangement has important implications. First, DNA extracted from broncho-alveolar lavage can inform on lung epithelial cell genome and epigenome. Second, pathologic disruption of tissue architecture as occurs in cancer can release lung DNA to blood, which can be detected on the background of a very low healthy baseline.

Indeed, we were able to detect our universal lung-specific methylation markers in the plasma of patients with advanced lung cancer. In addition, further study in patients undergoing bronchoscopy suggests that lung methylation markers can be identified in cfDNA as a biomarker of lung pathology, including cancer.

Perhaps the most interesting and unique aspect of lung methylation markers is their ability to report on non-cancer lung pathologies involving lung cell death, for which there are currently virtually no circulating biomarkers. Indeed, we found that patients with COPD release more lung cfDNA to blood during exacerbation of the disease, and that the levels of lung cfDNA in such patients predicts mortality.

In summary, this example describes the complete methylomes of lung alveolar and bronchial epithelial cells, and use the information present in these methylomes for developing circulating biomarkers, opening a novel minimally-invasive window into human lung turnover dynamics.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A method for identifying that a biological sample comprises DNA from a cell type, the method comprising:
detecting the methylation status of each of at least four CpG sites of a target DNA fragment in the biological sample; and
identifying the target DNA fragment as being:
(1) from a human oral, larynx or esophageal epithelial cell when no more than 40% of the CpG sites are methylated, wherein at least one of the CpG sites is located within a human genomic sequence selected from the group consisting of SEQ ID NO: 1-90, or when 50% or more of the CpG sites are methylated, wherein at least one of the CpG sites is located within a human genomic sequence selected from the group consisting of SEQ ID NO: 126-133;
(2) from a human gastric epithelial cell when no more than 40% of the CpG sites are methylated, wherein at least one of the CpG sites is located within a human genomic sequence selected from the group consisting of SEQ ID NO: 151-330, or when 50% c or more of the CpG sites are methylated, wherein at least one of the CpG sites is located within a human genomic sequence selected from the group consisting of SEQ ID NO: 379-401;
(3) from a human small intestine epithelial cell when no more than 40% of the CpG sites are methylated, wherein at least one of the CpG sites is located within a human genomic sequence selected from the group consisting of SEQ ID NO: 429-527, or when 50% or more of the CpG sites are methylated, wherein at least one of the CpG sites is located within a human genomic sequence selected from the group consisting of SEQ ID NO: 555-564;
(4) from a human colon epithelial cell when no more than 40% of the CpG sites are methylated, wherein at least one of the CpG sites is located within a human genomic sequence selected from the group consisting of SEQ ID NO: 580-657, or when 50% or more of the CpG sites are methylated, wherein at least one of the CpG sites is located within a human genomic sequence selected from the group consisting of SEQ ID NO: 705-715;
(5) from a human colon fibroblast when no more than 40% of the CpG sites are methylated, wherein at least one of the CpG sites is located within a human genomic sequence selected from the group consisting of SEQ ID NO: 730-732, or when 50% or more of the CpG sites are methylated, wherein at least one of the CpG sites is located within a human genomic sequence selected from the group consisting of SEQ ID NO: 733-739;
(6) from a human gallbladder epithelial cell when no more than 40% of the CpG sites are methylated, wherein at least one of the CpG sites is located within a human genomic sequence selected from the group consisting of SEQ ID NO: 742-829, or when 50% or more of the CpG sites are methylated, wherein at least one of the CpG sites is located within a human genomic sequence selected from the group consisting of SEQ ID NO: 868-875;
(7) from a human liver hepatocyte when no more than 40% of the CpG sites are methylated, wherein at least one of the CpG sites is located within a human genomic sequence selected from the group consisting of SEQ ID NO: 877-980, or when 50% or more of the CpG sites are methylated, wherein at least one of the CpG sites is located within a human genomic sequence selected from the group consisting of SEQ ID NO: 1003-1018;
(8) from a human pancreatic acinar cell when no more than 40% of the CpG sites are methylated, wherein at least one of the CpG sites is located within a human genomic sequence selected from the group consisting of SEQ ID NO: 1028-1112, or when 50% or more of the CpG sites are methylated, wherein at least one of the CpG sites is located within a human genomic sequence selected from the group consisting of SEQ ID NO: 1156-1161;
(9) from a human pancreatic alpha cell when no more than 40% of the CpG sites are methylated, wherein at least one of the CpG sites is located within a human genomic sequence selected from the group consisting of SEQ ID NO: 1181-1282, or when 50% or more of the CpG sites are methylated, wherein at least one of the CpG sites is located within a human genomic sequence selected from the group consisting of SEQ ID NO: 1307-1315;
(10) from a human pancreatic beta cell when no more than 40% of the CpG sites are methylated, wherein at least one of the CpG sites is located within a human genomic sequence selected from the group consisting of SEQ ID NO: 1332-1440, or when 50% or more of the CpG sites are methylated, wherein at least one of the CpG sites is located within a human genomic sequence selected from the group consisting of SEQ ID NO: 1461-1471;
(11) from a human pancreatic delta cell when no more than 40% of the CpG sites are methylated, wherein at least one of the CpG sites is located within a human genomic sequence selected from the group consisting of SEQ ID NO: 1486-1594, or when 50% or more of the CpG sites are methylated, wherein at least one of the CpG sites is located within a human genomic sequence selected from the group consisting of SEQ ID NO: 1614-1624; or
(12) from a human pancreatic ductal cell when no more than 40% of the CpG sites are methylated, wherein at least one of the CpG sites is located within a human genomic sequence selected from the group consisting of SEQ ID NO: 1639-1742, or when 50% or more of the CpG sites are methylated, wherein at least one of the CpG sites is located within a human genomic sequence selected from the group consisting of SEQ ID NO: 1768-1779.

2. The method of claim 1, further comprising detecting the methylation status of each of at least four CpG sites of a second target DNA fragment in the biological sample, and identifying the second target DNA fragment as being:
(1′) from a human oral, larynx or esophageal epithelial cell when no more than 40% of the CpG sites are methylated, wherein at least one of the CpG sites is located within a human genomic sequence selected from the group consisting of SEQ ID NO: 1-125, or when 50% or more of the CpG sites are methylated, wherein at least one of the CpG sites is located within a human genomic sequence selected from the group consisting of SEQ ID NO: 126-150;
(2′) from a human gastric epithelial cell when no more than 40% of the CpG sites are methylated, wherein at least one of the CpG sites is located within a human genomic sequence selected from the group consisting of SEQ ID NO: 151-378, or when 50% or more of the CpG sites are methylated, wherein at least one of the CpG sites is located within a human genomic sequence selected from the group consisting of SEQ ID NO: 379-428;
(3′) from a human small intestine epithelial cell when no more than 40% of the CpG sites are methylated, wherein at least one of the CpG sites is located within a human genomic sequence selected from the group consisting of SEQ ID NO: 429-554, or when 50% or more of the CpG sites are methylated, wherein at least one of the CpG sites is located within a human genomic sequence selected from the group consisting of SEQ ID NO: 555-579;
(4′) from a human colon epithelial cell when no more than 40% of the CpG sites are methylated, wherein at least one of the CpG sites is located within a human genomic sequence selected from the group consisting of SEQ ID NO: 580-704, or when 50% or more of the CpG sites are methylated, wherein at least one of the CpG sites is located within a human genomic sequence selected from the group consisting of SEQ ID NO: 705-729;
(5′) from a human colon fibroblast when no more than 40% of the CpG sites are methylated, wherein at least one of the CpG sites is located within a human genomic sequence selected from the group consisting of SEQ ID NO: 730-732, or when 50% or more of the CpG sites are methylated, wherein at least one of the CpG sites is located within a human genomic sequence selected from the group consisting of SEQ ID NO: 733-741;
(6′) from a human gallbladder epithelial cell when no more than 40% of the CpG sites are methylated, wherein at least one of the CpG sites is located within a human genomic sequence selected from the group consisting of SEQ ID NO: 742-867, or when 50% or more of the CpG sites are methylated, wherein at least one of the CpG sites is located within a human genomic sequence selected from the group consisting of SEQ ID NO: 868-876;
(7′) from a human liver hepatocyte when no more than 40% of the CpG sites are methylated, wherein at least one of the CpG sites is located within a human genomic sequence selected from the group consisting of SEQ ID NO: 877-1002, or when 50% or more of the CpG sites are methylated, wherein at least one of the CpG sites is located within a human genomic sequence selected from the group consisting of SEQ ID NO: 1003-1027;
(8′) from a human pancreatic acinar cell when no more than 40% of the CpG sites are methylated, wherein at least one of the CpG sites is located within a human genomic sequence selected from the group consisting of SEQ ID NO: 1028-1155, or when 50% or more of the CpG sites are methylated, wherein at least one of the CpG sites is located within a human genomic sequence selected from the group consisting of SEQ ID NO: 1156-1180;
(9′) from a human pancreatic alpha cell when no more than 40% of the CpG sites are methylated, wherein at least one of the CpG sites is located within a human genomic sequence selected from the group consisting of SEQ ID NO: 1181-1306, or when 50% or more of the CpG sites are methylated, wherein at least one of the CpG sites is located within a human genomic sequence selected from the group consisting of SEQ ID NO: 1307-1331;
(10′) from a human pancreatic beta cell when no more than 40% of the CpG sites are methylated, wherein at least one of the CpG sites is located within a human genomic sequence selected from the group consisting of SEQ ID NO: 1332-1460, or when 50% or more of the CpG sites are methylated, wherein at least one of the CpG sites is located within a human genomic sequence selected from the group consisting of SEQ ID NO: 1461-1485;
(11′) from a human pancreatic delta cell when no more than 40% of the CpG sites are methylated, wherein at least one of the CpG sites is located within a human genomic sequence selected from the group consisting of SEQ ID NO: 1486-1613, or when 50% or more of the CpG sites are methylated, wherein at least one of the CpG sites is located within a human genomic sequence selected from the group consisting of SEQ ID NO: 1614-1638; or
(12′) from a human pancreatic ductal cell when no more than 40% of the CpG sites are methylated, wherein at least one of the CpG sites is located within a human genomic sequence selected from the group consisting of SEQ ID NO: 1639-1767, or when 50% or more of the CpG sites are methylated, wherein at least one of the CpG sites is located within a human genomic sequence selected from the group consisting of SEQ ID NO: 1768-1792.

3. The method of claim 1, wherein the biological sample comprises blood, plasma, serum, semen, milk, urine, saliva or cerebral spinal fluid, acquired from a human subject.

4. The method of claim 3, wherein the target DNA fragment is a cell-free DNA fragment.

5. The method of claim 4, wherein identifying the cell-free DNA fragment as being from a cell type comprises detecting abnormal cell death of the cell type, or a disease relating to the cell type.

6. The method of claim 4, further comprising identifying the human subject as having or likely having an injury, inflammation, or cancer at the corresponding cell type.

7. The method of claim 5, further comprising identifying the human subject as having or likely having a pancreatic disease or condition when the amount of the cell-free DNA fragment identified as being from a pancreatic cell type is greater than a reference cut-off value.

8. The method of claim 7, wherein the pancreatic disease or condition is diabetes, inflammation, or cancer.

9. A method for identifying that a biological sample comprises DNA from a cell type, the method comprising:
detecting the methylation status of each of at least four CpG sites of a target DNA fragment in the biological sample; and
identifying the target DNA fragment as being:
(1) from a human endometrium epithelial cell when no more than 40% of the CpG sites are methylated, wherein at least one of the CpG sites is located within a human genomic sequence selected from the group consisting of SEQ ID NO: 1793-1864, or when 50% or more of the CpG sites are methylated, wherein at least one of the CpG sites is located within a human genomic sequence selected from the group consisting of SEQ ID NO: 1893-1905;
(2) from a human fallopian epithelial cell when no more than 40% of the CpG sites are methylated, wherein at least one of the CpG sites is located within a human genomic sequence selected from the group consisting of SEQ ID NO: 1918-2022, or when 50% or more of the CpG sites are methylated, wherein at least one of the CpG sites is located within a human genomic sequence selected from the group consisting of SEQ ID NO: 2043-2061;
(3) from a human kidney epithelial cell when no more than 40% of the CpG sites are methylated, wherein at least one of the CpG sites is located within a human genomic sequence selected from the group consisting of SEQ ID NO: 2068-2141, or when 50% or more of the CpG sites are methylated, wherein at least one of the CpG sites is located within a human genomic sequence selected from the group consisting of SEQ ID NO: 2195-2209;
(4) from a human bladder epithelial cell when no more than 40% of the CpG sites are methylated, wherein at least one of the CpG sites is located within a human genomic sequence selected from the group consisting of SEQ ID NO: 2220-2298, or when 50% or more of the CpG sites are methylated, wherein at least one of the CpG sites is located within a human genomic sequence selected from the group consisting of SEQ ID NO: 2346-2350;
(5) from a human prostate epithelial cell when no more than 40% of the CpG sites are methylated, wherein at least one of the CpG sites is located within a human genomic sequence selected from the group consisting of SEQ ID NO: 2371-2476, or when 50% or more of the CpG sites are methylated, wherein at least one of the CpG sites is located within a human genomic sequence selected from the group consisting of SEQ ID NO: 2496-2500;
(6) from a human breast basal epithelial cell when no more than 40% of the CpG sites are methylated, wherein at least one of the CpG sites is located within a human genomic sequence selected from the group consisting of SEQ ID NO: 2521-2616, or when 50% or more of the CpG sites are methylated, wherein at least one of the CpG sites is located within a human genomic sequence selected from the group consisting of SEQ ID NO: 2652-2659; or
(7) from a human breast luminal epithelial cell when no more than 40% of the CpG sites are methylated, wherein at least one of the CpG sites is located within a human genomic sequence selected from the group consisting of SEQ ID NO: 2677-2748, or when 50% or more of the CpG sites are methylated, wherein at least one of the CpG sites is located within a human genomic sequence selected from the group consisting of SEQ ID NO: 2803-2815.

10. The method of claim 9, wherein the biological sample comprises blood, plasma, serum, semen, milk, urine, saliva or cerebral spinal fluid, acquired from a human subject.

11. The method of claim 10, wherein the target DNA fragment is a cell-free DNA fragment.

12. The method of claim 11, wherein identifying the cell-free DNA fragment as being from a cell type indicates abnormal cell death of the cell type, or a disease relating to the cell type.

13. The method of claim 11, further comprising identifying the human subject as having or likely having an injury, inflammation, or cancer at the corresponding genito-urinary cell.

14. A method for identifying that a biological sample comprises DNA from a cell type, the method comprising:
detecting the methylation status of each of at least four CpG sites of a target DNA fragment in the biological sample; and
identifying the target DNA fragment as being:
(1) from a human lung alveolar epithelial cell when no more than 40% of the CpG sites are methylated, wherein at least one of the CpG sites is located within a human genomic sequence selected from the group consisting of SEQ ID NO: 2828-2899, or when 50% or more of the CpG sites are methylated, wherein at least one of the CpG sites is located within a human genomic sequence selected from the group consisting of SEQ ID NO: 2954-2960;
(2) from a human lung bronchial epithelial cell when no more than 40% of the CpG sites are methylated, wherein at least one of the CpG sites is located within a human genomic sequence selected from the group consisting of SEQ ID NO: 2979-3087, or when 50% or more of the CpG sites are methylated, wherein at least one of the CpG sites is located within a human genomic sequence selected from the group consisting of SEQ ID NO: 3105-3109;
(3) from a human heart cardiomyocyte when no more than 40% of the CpG sites are methylated, wherein at least one of the CpG sites is located within a human genomic sequence selected from the group consisting of SEQ ID NO: 3130-3223, or when 50% or more of the CpG sites are methylated, wherein at least one of the CpG sites is located within a human genomic sequence selected from the group consisting of SEQ ID NO: 3255-3266;
(4) from a human heart fibroblast when no more than 40% of the CpG sites are methylated, wherein at least one of the CpG sites is located within a human genomic sequence selected from the group consisting of SEQ ID NO: 3280-3394, or when 50% or more of the CpG sites are methylated, wherein at least one of the CpG sites is located within a human genomic sequence selected from the group consisting of SEQ ID NO: 3408-3414; or
(5) from a human vascular endothelial cell when no more than 40% of the CpG sites are methylated, wherein at least one of the CpG sites is located within a human genomic sequence selected from the group consisting of SEQ ID NO. 3433-3547, or when 50% or more of the CpG sites are methylated, wherein at least one of the CpG sites is located within a human genomic sequence selected from the group consisting of SEQ ID NO: 3560-3579.

15. The method of claim 14, wherein the biological sample comprises blood, plasma, serum, semen, milk, urine, saliva or cerebral spinal fluid, acquired from a human subject.

16. The method of claim 15, wherein the target DNA fragment is a cell-free DNA fragment.

17. The method of claim 16, wherein identifying the cell-free DNA fragment as being from a cell type indicates abnormal cell death of the cell type, or a disease at the corresponding cardio-vascular-pulmonary cell.

18. The method of claim 1, further comprising detecting a genetic variation in the target DNA fragment, thereby determining that the cell from which the target DNA fragment is released contains the genetic variation.

19. The method of claim 5, further comprising administering to the patient an agent useful for treating the identified disease or condition.
